(19) United States
(12) Patent Application Publication
(10) Pub. No.: US 2025/0179078 A1
(43) Pub. Date: Jun. 5, 2025

(54) INHIBITORS OF NLRP3

(71) Applicant: PTC THERAPEUTICS, INC., Warren, NJ (US)

(72) Inventors: Xiaoyan ZHANG, Belle Mead, NJ (US); Rauful ALAM, Middlesex, NJ (US); Lauren BEJCEK, Englewood, NJ (US); Bradley B. GILBERT, Milford, NJ (US); Seyedmorteza HOSSEYNI, Highland Park, NJ (US); Eduardo HUARTE, Wilmington, DE (US); Jing LI, Skillman, NJ (US); Yao LIU, Somerville, NJ (US); Kyle NIEDERER, Titusville, NJ (US); Erica N. PARKER, New Brunswick, NJ (US); Meenu PILLAI, Clarksburg, MD (US); Ettore RASTELLI, Jersey City, NJ (US); Nadiya SYDORENKO, Princeton, NJ (US); Nanjing ZHANG, Princeton, NJ (US); Tianyi ZHENG, Highland Park, NJ (US); Matthew G. WOLL, Dunellen, NJ (US); Handoko -, Newark, NJ (US)

(21) Appl. No.: 18/839,229

(22) PCT Filed: Feb. 16, 2023
(86) PCT No.: PCT/US2023/062771

§ 371 Date: Aug. 16, 2024

Related U.S. Application Data
(60) Provisional application No. 63/311,463, filed on Feb. 18, 2022.

(51) Int. Cl. C07D 487/04; A61K 31/53

(52) U.S. Cl. CPC C07D 487/04; A61K 31/53

(57) ABSTRACT

The present invention relates to compounds that are useful as inhibitors of NOD-like receptor protein 3 (NLRP3) inflammasome pathway, processes for the preparation of said compounds, pharmaceutical compositions comprising said compounds, methods of using said compounds in the treatment of various diseases and disorders, and medicaments containing them, and their use in diseases and disorders mediated by NLRP3.

### CROSS-REFERENCE TO RELATED APPLICATIONS

This application claims the benefit of, and priority to PCT application number PCT/US2022/075421, filed Aug. 24, 2022, and U.S. Provisional Patent Application No. 63/311,463 filed on Feb. 18, 2022, the contents of which are herein incorporated by reference in their entirety and for all purposes.

### FIELD OF THE INVENTION

The present invention relates to compounds that are useful as inhibitors of NOD-like receptor protein 3 (NLRP3) inflammasome pathway. The present invention also relates to processes for the preparation of said compounds, pharmaceutical compositions comprising said compounds, methods of using said compounds in the treatment of various diseases and disorders, and medicaments containing them, and their use in diseases and disorders mediated by NLRP3.

### BACKGROUND

The term of inflammasome was coined by Martinon et al. to describe the molecular platform triggering activation of inflammatory caspases and processing of interleukin 1 (IL-1) family cytokines (Fabio Martinon et al., Mol Cell 10(2):417-26, 2002). Inflammasomes are part of the innate immune system. Inflammasome activation is initiated by assembling of a multiprotein complex, including nucleotide binding oligomerization domain (NOD)-like receptor (NLR), the adapter apoptosis-associated speck-like protein containing a caspase recruitment domain (ASC), and the effector protease caspase-1. The assemble of the complex results in the activation of caspase-1 and the release of the mature proinflammatory cytokines, such as IL-1β and IL-18.

Among inflammasomes, the NLR family NACHT, LRR and PYD domains-containing protein 3 (NLRP3) inflammasome has been studied extensively and was found to be activated by a wide spectrum of stimuli. The regulatory mechanisms of NLRP3 activation are summarized in a recent review paper (Seungwha Paik et al., Cell Mol Immunol 18(5):1141-1160, 2021).

NLRP3 activation is triggered by various infectious, non-infectious molecules, including molecular byproducts of aging, physical inactivity and overnutrition. Once activated, it boosts the downstream production of the inflammatory cytokines IL-1β and IL-18. Gain-of function mutations of NLRP3 are associated with several genetic disorders including cryopyrin-associated periodic syndromes (CAPS). Additionally, NLRP3 is implicated in numerous common I) autoimmune, II) autoinflammatory, III) neurodegenerative, IV) cardiovascular and V) neuromuscular and muscular degenerative diseases e.g. (Matthew S J Mangan et al., Nat Rev Drug Discov 17(8):588-606, 2018; Corcoran et al., Pharmacol Rev 73(3):968-1000, 2021; Dubuisson et al., Cells 10(11):3023, 2021). Inflammasome activation has also been identified in retinal pigment epithelium (RPE) cells and proposed to be a causal factor for RPE dysfunction and degeneration (Gao et al., Mediators Inflamm 2015:690243, 2015). Further, NLRP3 activation is associated with severe COVID-19 cases and cytokine release syndrome (CRS) caused by cell-based therapeutics and biologic treatments (Tracey L Freeman and Talia H Swartz Front Immunol 11:1518, 2020; Lin et al., PLoS Pathog 6; 15(6):e1007795, 2019).

Therefore, an NLRP3 inflammasome inhibitor could be used as a single or combination of agents clinically as novel therapies for these diseases. Thus, there is a need for inhibitors of the NLRP3 inflammasome pathway to provide new and/or alternative treatments for these inflammasome-related diseases, disorders, such as autoinflammatory fever syndrome cryopyrin-associated periodic syndrome (CAPS), sickle cell disease, chronic liver disease, nonalcoholic steatohepatitis (NASH), gout, hyperoxaluria, pseudogout (chondrocalcinosis), Type I/Type II diabetes and related complications (e.g. nephropathy, retinopathy), fibrosis, rheumatoid arthritis, inflammatory bowel diseases, asthma and allergic airway inflammation, neuroinflammation-related disorders (e.g. multiple sclerosis, brain infection, acute injury, Alzheimer's disease, Parkinson's disease, Huntington's disease), neuromuscular and muscular degenerative diseases, atherosclerosis and cardiovascular risk (e.g. cardiovascular risk reduction (CvRR), hypertension), hidradenitis suppurativa, wound healing and scar formation, and cancer (e.g. colon cancer, lung cancer, myeloproliferative neoplasms, leukemias, myelodysplastic syndromes (MD), myelofibrosis).

### REFERENCES

Fabio Martinon, Kimberly Burns, Jurg Tschopp The inflammasome: a molecular platform triggering activation of inflammatory caspases and processing of proIL-beta Mol Cell 10(2):417-26, 2002.
Seungwha Paik, Jin Kyung Kim, Prashanta Silwal, Chihiro Sasakawa, Eun-Kyeong Jo An update on the regulatory mechanisms of NLRP3 inflammasome activation Cell Mol Immunol 18(5):1141-1160, 2021.
Matthew S J Mangan, Edward J Olhava, William R Roush, H Martin Seidel, Gary D Glick, Eicke Latz Targeting the NLRP3 inflammasome in inflammatory diseases Nat Rev Drug Discov 17(8):588-606, 2018
Sarah Corcoran, Reena Halai, Matthew A Coope Pharmacological Inhibition of the Nod-Like Receptor Family Pyrin Domain Containing 3 Inflammasome with MCC950 Pharmacol Rev 73(3):968-1000, 2021
Nicolas Dubuisson, Romain Versele, Maria A Davis-López de Carrizosa, Camille M Selvais, Sonia M Brichard, Michel Abou-Samra Walking down Skeletal Muscle Lane: From Inflammasome to Disease Cells 10(11):3023, 2021
Jiangyuan Gao, Ruozhou Tom Liu, Sijia Cao, Jing Z Cui, Aikun Wang, Eleanor To, Joanne A Matsubara NLRP3 inflammasome: activation and regulation in age-related macular degeneration Mediators Inflamm 2015:690243, 2015
Tracey L Freeman and Talia H Swartz Targeting the NLRP3 Inflammasome in Severe COVID-19 Front Immunol 11:1518, 2020
Lan Lin, Lei Xu, Weihua Lv, Li Han, Yaozu Xiang, Lei Fu, Meilin Jin, Rui Zhou, Huanchun Chen, Anding Zhang An NLRP3 inflammasome-triggered cytokine storm contributes to Streptococcal toxic shock-like syndrome (STSLS)PLoS Pathog 6; 15(6):e1007795, 2019

### SUMMARY OF THE INVENTION

The invention provides compounds or pharmaceu-tically acceptable salts thereof, pharmaceutical compositions thereof, which compounds inhibit the NLRP3 inflammasome pathway. The invention further provides methods of treating, or preventing, disease and/or disorders related to NLRP3, comprising administering to a subject in need thereof an effective amount of the compounds of the invention, or a pharmaceutically acceptable salt thereof.

Various aspects of the invention are described herein.

The instant application discloses a compound of Formula I:

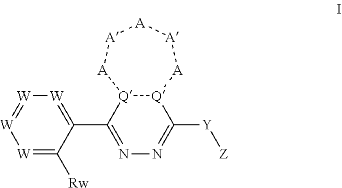

wherein:
R_{w} is hydrogen, C₁₋₆alkyl (e.g., CH₃), C₂₋₈alkynyl, halogen (e.g., F, Cl), C₁₋₆alkoxy (e.g., OCH₃), halo-C₁₋₄alkyl (e.g., CHF₂, CF₃), halo-C₁₋₄alkoxy (e.g., OCHF₂, OCF₃), cyano, —NH₂, —N(C₁₋₆alkyl)₂, thiol, —SO₂NH₂, —SO₂N(C₁₋₆alkyl)₂, —S(═O)(C₁₋₆alkyl), cycloalkyl, CHR₁ₐ, (C═O)R₁ₐ, OR₁ₐ, NR_{1b}, S(═O)R₁ₐ, S(═O)₂R₁ₐ, O(C═O)R₁ₐ, (C═O)OR₁ₐ, NR_{1b}(C═O)R_{1b}, (C═O)NR_{1b}, (C═O)N(R_{1b})₂ NR_{1b}(C═O)OR₁ₐ, O(C═O)NR_{1b}, ONR_{1b}(C═NR_{1b})NR_{1b}, NR_{1b}S(═O)₂ or S(═O)₂NR_{1b}, wherein each C₁₋₆alkyl, heterocycle (e.g., 2, 5-dihydrofuran-3-yl), heteroaryl and aryl are optionally substituted with 1 or 2 substituents each selected from R₅;
each W is independently CH, CR′ or N;
each Q′ is independently N, C or CH;
each A is independently CH, CH₂, CRₐ, CHRₐ, CR₄, CHR₄, N, NH, NRₐ, NR₄, S, or O;
each A′ is independently absent, CH, CH₂, CRₐ, CHRₐ, CR₄, CHR₄, N, NH, NRₐ, NR₄, S, or O;
each Rₐ is independently hydrogen, deuterium, halogen, —CN, —OH, —OR₂, ═O, ═N—OR₂, —SR₂, —S(═O)R₂, —S(═O)₂R₂, —N(R₂)₂, —NR₂S(═O)(═NR₂)R₂, —NR₂S(═O)₂R₂, —S(═O)₂N(R₂)₂, —C(═O)R₂, —OC(═O)R₂, —C(═O)OR₂, —OC(═O)OR₂, —C(═O)N(R₂)₂, —OC(═O)N(R₂)₂, —NR₂C(═O)R₂, —P(═O)(R₂)₂, C₁₋₄alkyl, (C₁₋₄alkyl)₂, halo-C₁₋₆alkyl, C₁₋₆heteroalkyl, C₃₋₈cycloalkyl, C₂₋₇heterocycloalkyl, aryl or monocyclic heteroaryl;
each—represents either an absent, single, or double bond;
Y is C(R₁ₐ)₂, C═O, O, NR_{1b}, or a bond;
each R₁ₐ is independently hydrogen, halogen, hydroxyl, cyano, C₁₋₄alkyl, deutero-C₁₋₄alkyl, halo-C₁₋₄alkyl, amino or hydroxy-C₁₋₄alkyl, C₃₋₁₀cycloalkyl, C₂₋₇heterocycloalkyl or aryl; each R_{1b} is independently hydrogen, C₁₋₄alkyl, deutero-C₁₋₄alkyl, halo-C₁₋₄alkyl or hydroxy-C₁₋₄alkyl;
each R′ is independently heterocyclyl, heteroaryl, aryl, C₃₋₈cycloalkyl, C₁₋₄alkyl, C₁₋₄alkoxy, deutero-C₁₋₄alkyl, halo-C₁₋₄alkyl (e.g., trifluoromethyl, difluoromethyl), halo-C₁₋₄alkoxy (e.g., trifluoromethoxy), hydroxy-C₁₋₄alkyl, halogen, hydroxy or cyano,
each Z is heterocyclyl, heteroaryl, aryl, C₃₋₁₀cycloalkyl, C₁₋₄alkyl, deutero-C₁₋₄alkyl, halo-C₁₋₄alkyl, hydroxy-C₁.-alkyl, C₁₋₆alkoxy, NH(hydroxy-C₁₋₆alkyl), NH(C₁₋₆alkoxy) wherein each Z is optionally substituted with OH, NH₂, —CO₂H, halogen, C₁₋₆alkyl, C₁₋₆haloalkyl, C₁₋₆ hydroxyalkyl, C₂₋₆acyl, C₂₋₆alkanoic acid, C₂₋₆alkanoate ester, or heterocyclyl, and wherein heterocyclyl, C₃₋₁₀cycloalkyl is a saturated or partially unsaturated 3-7 membered monocyclic, 6-10 membered bicyclic or 13-16 membered polycyclic ring system having 1, 2, or 3 heteroatom ring members independently selected from N, O, and S, each optionally substituted with 1, 2, 3, 4, or 5 substituents each selected from R₂; R₂ is independently selected from halogen, hydroxyl, cyano, C₁₋₄alkyl, hydroxy-C₁₋₄alkyl, deuterium-C₁₋₄alkyl, halo-C₁₋₄alkyl, amino, C₁₋₄alkylamino, (C₁-alkyl)₂amino, halo-C₁₋₄ alkylamino, (halo-C₁₋₆alkyl)₂amino, hydroxy-C₁₋₄alkylamino, C₁₋₄alkoxy-C₁₋₄alkyl-amino, aminoC₁₋₄alkyl, C₁₋₄alkylaminoC₁₋₄alkyl, (C₁₋₄alkylamino)₂C₁₋₄alkyl, C₁₋₄alkoxy, halo-C₁₋₄alkoxy, hydroxy-C₁₋₄alkoxy, C₁₋₄alkyl-C₁₋₄alkoxy, C₃₋₁₀cycloalkyl, C₃₋₁₀cycloalkyl-amino, C₃₋₁₀cycloalkyl-amino-C₁₋₄alkyl, heteroarylC₁₋₄alkyl, heteroarylamino, heteroarylC₁₋₄alkyl-amino, heterocyclyl, heterocyclyl-C₁₋₄alkyl, heterocyclyl-amino, heterocyclyl-amino-C₁₋₄alkyl, heterocyclyl-C₁₋₄alkoxy, heterocyclyl-amino-C₃₋₁₀cycloalkyl, phenyl, and phenyl-C₁₋₄alkoxy,
wherein heteroaryl is a 5-6 membered monocyclic or 6-10 membered bicyclic ring system having 1, 2, 3, or 4 heteroatom ring members independently selected from N, O, or S,
wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S,
wherein C₃₋₁₀cycloalkyl is a saturated or partially unsaturated 3-7 membered monocyclic ring system, and
wherein each instance of phenyl, heteroaryl, heterocyclyl, or C₃₋₁₀cycloalkyl is optionally substituted with 1 or 2 substituents each selected from R₃;
R₃ is independently selected from halogen, hydroxyl, cyano, C₁₋₄alkyl, deutero-C₁₋₄alkyl, halo-C₁₋₄alkyl, amino, C₁₋₄alkoxy, and halo-C₁₋₄alkoxy;
each R₄ is independently selected from halogen, hydroxyl, cyano, C₁₋₄alkyl, deutero-C₁₋₄alkyl, halo-C₁₋₄alkyl, amino, C₁₋₄alkylamino, (C₁₋₄alkyl)₂amino, C₁₋₄alkoxy, halo-C₁₋₄alkoxy, heteroaryl, heterocyclyl, and phenyl,
wherein heteroaryl is a 5-6 membered monocyclic or 9-10 membered bicyclic ring system having 1, 2, 3, or 4 heteroatom ring members selected from N, O, and S,
wherein heterocyclyl is a saturated or partially unsaturated 3-7 membered monocyclic or 9-10 membered bicyclic ring system having 1, 2, or 3 heteroatom ring members selected from N, O, and S, and
wherein each instance of phenyl, heteroaryl or heterocyclyl is optionally substituted with 1, or 2 substituents each selected from R₅;
R₅ is independently selected from halogen, hydroxyl, cyano, nitro, C₁₋₄alkyl, deutero-C₁₋₄alkyl, halo-C₁₋₄alkyl, amino, C₁₋₄alkylamino, (C₁₋₄alkyl)₂amino, aminoC₁₋₄alkyl, hydroxy-C₁₋₄alkyl, C₁₋₄alkylcarbonyl, C₁₋₄alkoxy, C₁₋₄alkylthio, halo-C₁₋₄alkoxy, and C₃₋₁₀cycloalkyl;
wherein a form of the compound is selected from the group consisting of a pharmaceutically acceptable salt, hydrate, solvate, racemate, enantiomer, diastereomer, stereoisomer, and tautomer form thereof.

Another aspect of the invention provides a compound having the structure of Formula II:

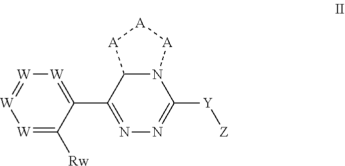

wherein:
each W is independently CH, CR′ or N;
each A is independently CH, CH₂, CRₐ, CR₄, N, NH or NR₄;
each—represents either a single, or double bond;
Y is NR_{1b} or a bond;
each R_{w} is hydrogen, halogen, C₁₋₄alkyl, C₃₋₁₀cycloalkyl, C₃₋₁₀cycloalkoxy, halo-C₁₋₄alkyl, C₁₋₄alkoxy, or halo-C₁₋₄alkoxy,
each R_{1b} is independently hydrogen, C₁₋₄alkyl, deutero-C₁₋₄alkyl, halo-C₁₋₄alkyl or hydroxy-C₁₋₄alkyl;
each Z is heterocyclyl, heteroaryl, aryl, C₃₋₁₀cycloalkyl, C₁₋₄alkyl, deutero-C₁₋₄alkyl, halo-C₁₋₄alkyl, hydroxy-C₁₋₈alkyl, NH(hydroxy-C₁₋₆alkyl), NH(C₁₋₆alkoxy) wherein each Z is optionally substituted with OH, NH₂, —CO₂H, halogen, C₁₋₆alkyl, C₁₋₆haloalkyl, C₁₋₆ hydroxyalkyl, C₂₋₆acyl, C₂₋₆alkanoic acid, C₂₋₆alkanoate ester, or heterocyclyl, and wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S, each optionally substituted with 1, 2, 3, 4, or 5 substituents each selected from R₂;
R₂ is independently selected from halogen, hydroxyl, cyano, C₁₋₄alkyl, deutero-C₁₋₄alkyl, halo-C₁₋₄alkyl, amino, C₁₋₄alkylamino, (C₁₋₆alkyl)₂amino, halo-C₁₋₄alkylamino, (halo-C₁₋₆alkyl)₂amino, hydroxy-C₁₋₄alkylamino, C₁₋₄alkoxy-C₁₋₄alkyl-amino, aminoC₁₋₄alkyl, C₁₋₄alkylaminoC₁₋₄alkyl, (C₁₋₄alkylamino)₂C₁₋₄alkyl, C₁₋₄alkoxy, halo-C₁₋₄alkoxy, C₁₋₄alkyl-C₁₋₄alkoxy, C₃₋₁₀cycloalkyl, C₃₋₁₀cycloalkyl-amino, C₃₋₁₀cycloalkyl-amino-C₁₋₄alkyl, heteroarylC₁₋₄alkyl, heteroarylamino, heteroarylC₁₋₄alkyl-amino, heterocyclyl, heterocyclyl-C₁₋₄alkyl, heterocyclyl-amino, heterocyclyl-amino-C₁₋₄alkyl, heterocyclyl-C₁₋₄alkoxy, heterocyclyl-amino-C₃₋₁₀cycloalkyl, phenyl, and phenyl-C₁₋₄alkoxy,
wherein heteroaryl is a 5-6 membered monocyclic or 6-10 membered bicyclic ring system having 1, 2, 3, or 4 heteroatom ring members independently selected from N, O, and S,
wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S,
wherein C₃₋₁₀cycloalkyl is a saturated or partially unsaturated 3-7 membered monocyclic ring system,
wherein each instance of phenyl, heteroaryl, heterocyclyl, or C₃₋₁₀cycloalkyl is optionally substituted with 1 or 2 substituents each selected from R₃;
R₃ is independently selected from halogen, hydroxyl, cyano, C₁₋₄alkyl, deutero-C₁₋₄alkyl, halo-C₁₋₄alkyl, amino, C₁₋₄alkoxy, and halo-C₁₋₄alkoxy;
R′ is selected from H, substituted or unsubstituted cycloalkyl, C₁₋₄alkyl, halo-C₁₋₄alkyl, halo-C₁₋₆alkoxy, halogen, C₁₋₆alkoxy, cyano, hydroxy-C₁₋₄alkyl or aryl optionally substituted by C₁₋₄alkyl;
each Rₐ is independently H, deuterium, halogen, —CN, —OH, —OR₂, ═O, ═N—OR₂, —SR₂, —S(═O)R₂, —S(═O)₂R₂, —N(R₂)₂, —NR₂S(═O)(═NR₂)R₂, —NR₂S(═O)₂R₂, —S(═O)₂N(R₂)₂, —C(═O)R₂, —OC(═O)R₂, —C(═O)OR₂, —OC(═O)OR₂, —C(═O)N(R₂)₂, —OC(═O)N(R₂)₂, —NR₂C(═O)R₂, —P(═O)(R₂)₂, C₁₋₄alkyl, (C₁₋₄alkyl)₂, halo-C₁₋₆alkyl, C₁₋₆heteroalkyl, C₃₋₈cycloalkyl, C₂₋₇heterocycloalkyl, aryl or monocyclic heteroaryl; and
R₄ is independently selected from halogen, hydroxyl, cyano, or C₁₋₄alkyl;
wherein a form of the compound is selected from the group consisting of a pharmaceutically acceptable salt, hydrate, solvate, racemate, enantiomer, diastereomer, stereoisomer, tautomer, and isotope enriched form thereof.

The invention provides a pharmaceutical composition comprising a therapeutically effective amount of a compound according to the definition of the compound of Formula I or a form thereof, as disclosed herein, or a pharmaceutically acceptable salt thereof, and one or more pharmaceutically acceptable carriers. The pharmaceutical composition is useful in the treatment of diseases and/or disorders related to the NLRP3 activity. In another aspect, the invention provides a combination, in particular a pharmaceutical combination, comprising a therapeutically effective amount of a compound according to the definition of compounds of Formula I, or sub-Formula thereof, as disclosed herein, or a pharmaceutically acceptable salt thereof, and one or more therapeutic agents. In another aspect, the invention provides a combination, in particular a pharmaceutical combination, as disclosed herein, for use as a medicament.

In another aspect, the invention provides a compound of Formula I, or sub-Formula thereof, as disclosed herein, or a pharmaceutically acceptable salt thereof, for use in the treatment of a disease or disorder in which the NLRP3 signaling contributes to the pathology, and/or symptoms, and/or progression, of said disease or disorder. In another aspect, the invention provides a method of treating a disease or disorder in which the NLRP3 signaling contributes to the pathology, and/or symptoms, and/or progression, of said disease or disorder, comprising administering a therapeutically effective amount of a compound of Formula I, or sub-Formula thereof, as disclosed herein, or a pharmaceutically acceptable salt thereof. In another aspect, the invention provides a method of inhibiting the NLRP3 inflammasome activity in a subject in need thereof, the method comprises administering to the subject in need thereof a therapeutically effective amount of a compound of Formula I or form thereof, as disclosed herein, or a pharmaceutically acceptable salt thereof.

Another aspect of the invention relates to the use of a compound of Formula I, or sub-Formula thereof, as disclosed herein, or a pharmaceutically acceptable salt thereof, as a medicament.

Another aspect of the invention relates to a compound of Formula I, or sub-Formula thereof, as disclosed herein, or a pharmaceutically acceptable salt thereof, for use as a medicament.

Another aspect of the invention also provides a compound of Formula I, or sub-Formula thereof, as disclosed herein, or a pharmaceutically acceptable salt thereof, for use in the treatment of a disease or disorder selected from inflammasome-related disease/disorders, immune diseases, inflammatory diseases, auto-immune diseases, and auto-inflammatory diseases.

### DETAILED DESCRIPTION OF THE INVENTION

The invention provides compounds selected from:

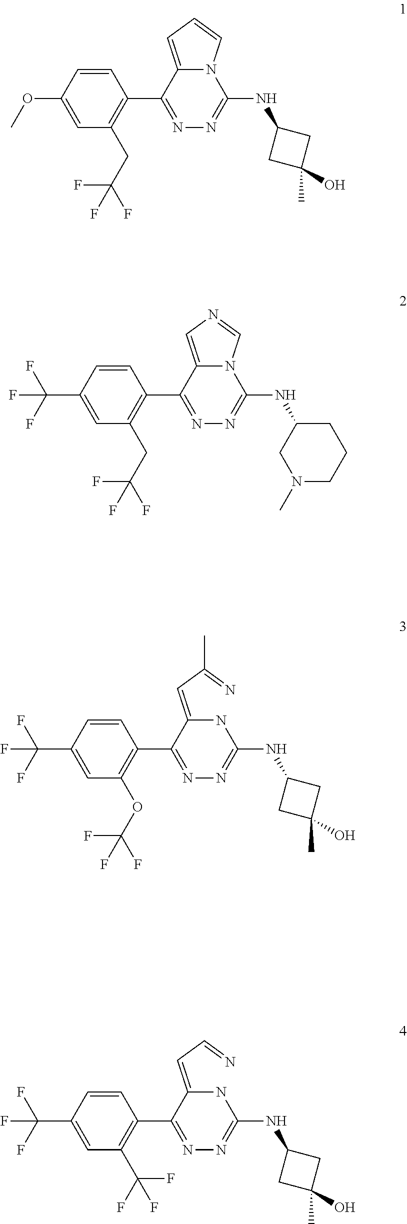 [SMILES: COc1ccc(-c2nnc(N[C@H]3C[C@@](C)(O)C3)n3cccc23)c(CC(F)(F)F)c1 ; CN1CCC[C@@H](Nc2nnc(-c3ccc(C(F)(F)F)cc3CC(F)(F)F)c3cncn23)C1 ; CC(=N)/C=C1\NC(N[C@H]2C[C@@](C)(O)C2)=NN=C1c1ccc(C(F)(F)F)cc1OC(F)(F)F ; C[C@]1(O)C[C@@H](NC2=NN=C(c3ccc(C(F)(F)F)cc3C(F)(F)F)/C(=C/C=N)N2)C1]

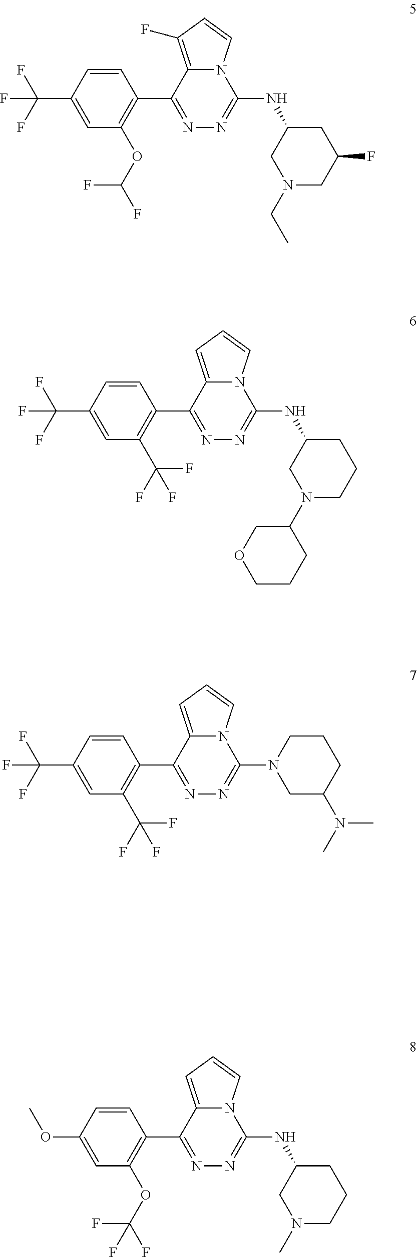

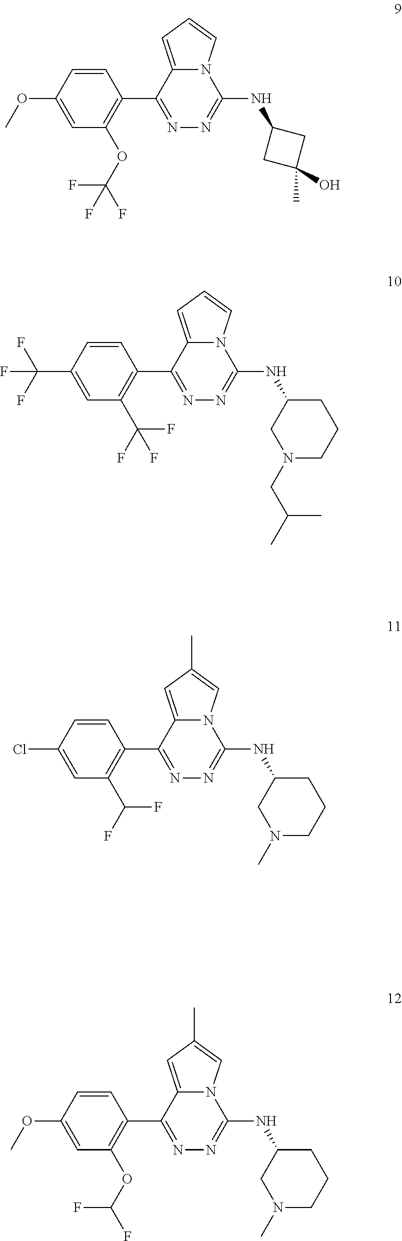 [SMILES: COc1ccc(-c2nnc(N[C@H]3C[C@@](C)(O)C3)n3cccc23)c(OC(F)(F)F)c1 ; CC(C)CN1CCC[C@@H](Nc2nnc(-c3ccc(C(F)(F)F)cc3C(F)(F)F)c3cccn23)C1 ; Cc1cc2c(-c3ccc(Cl)cc3C(F)F)nnc(N[C@@H]3CCCN(C)C3)n2c1 ; COc1ccc(-c2nnc(N[C@@H]3CCCN(C)C3)n3cc(C)cc23)c(OC(F)F)c1]

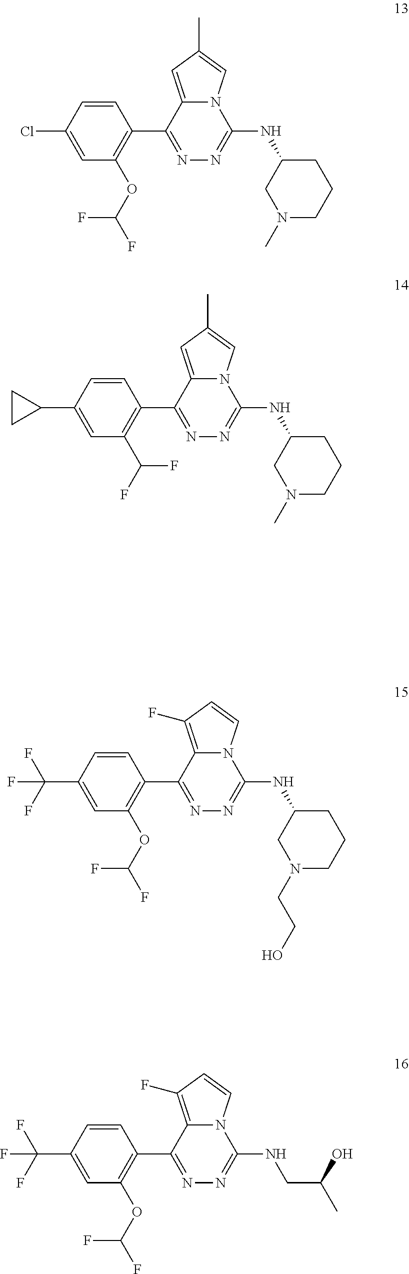 [SMILES: Cc1cc2c(-c3ccc(Cl)cc3OC(F)F)nnc(N[C@@H]3CCCN(C)C3)n2c1 ; Cc1cc2c(-c3ccc(C4CC4)cc3C(F)F)nnc(N[C@@H]3CCCN(C)C3)n2c1 ; OCCN1CCC[C@@H](Nc2nnc(-c3ccc(C(F)(F)F)cc3OC(F)F)c3c(F)ccn23)C1 ; C[C@H](O)CNc1nnc(-c2ccc(C(F)(F)F)cc2OC(F)F)c2c(F)ccn12]

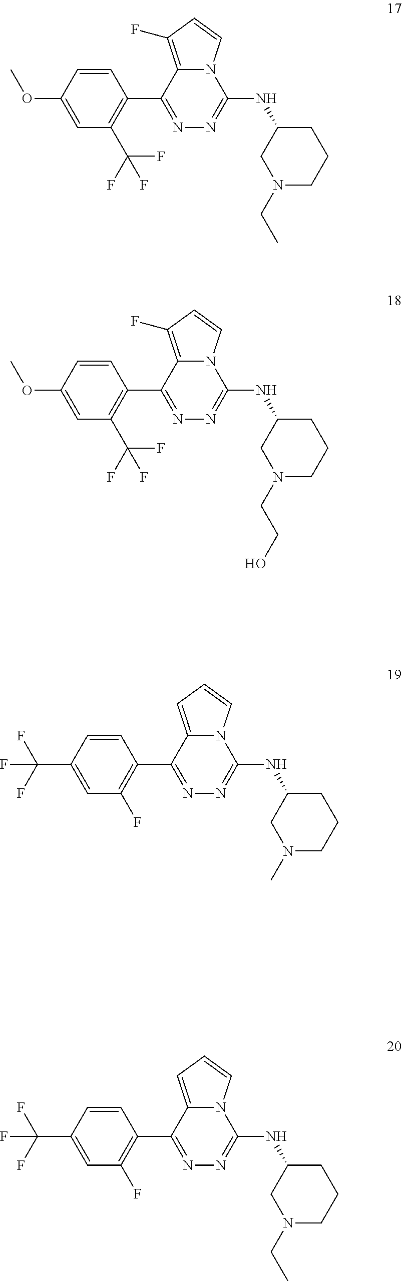 [SMILES: CCN1CCC[C@@H](Nc2nnc(-c3ccc(OC)cc3C(F)(F)F)c3c(F)ccn23)C1 ; COc1ccc(-c2nnc(N[C@@H]3CCCN(CCO)C3)n3ccc(F)c23)c(C(F)(F)F)c1 ; CN1CCC[C@@H](Nc2nnc(-c3ccc(C(F)(F)F)cc3F)c3cccn23)C1 ; CCN1CCC[C@@H](Nc2nnc(-c3ccc(C(F)(F)F)cc3F)c3cccn23)C1]

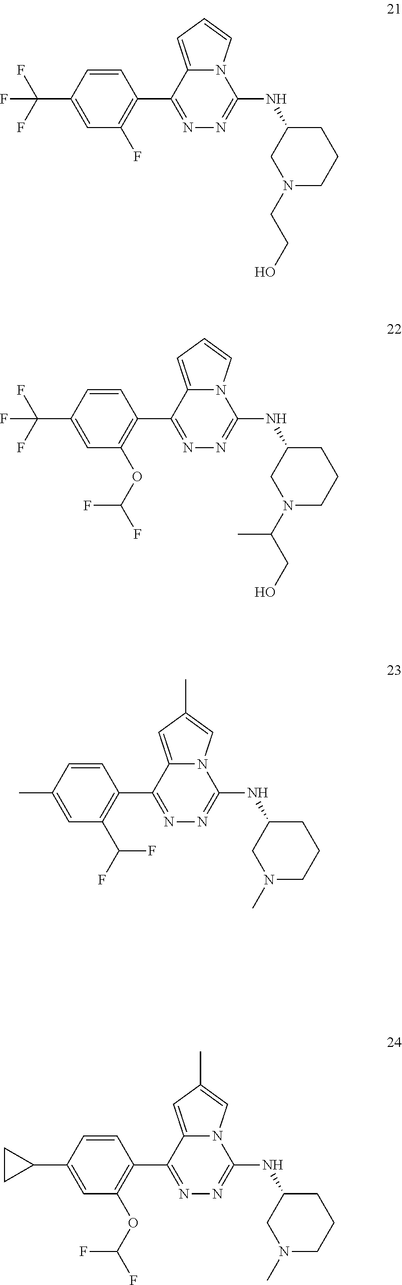 [SMILES: OCCN1CCC[C@@H](Nc2nnc(-c3ccc(C(F)(F)F)cc3F)c3cccn23)C1 ; CC(CO)N1CCC[C@@H](Nc2nnc(-c3ccc(C(F)(F)F)cc3OC(F)F)c3cccn23)C1 ; Cc1ccc(-c2nnc(N[C@@H]3CCCN(C)C3)n3cc(C)cc23)c(C(F)F)c1 ; Cc1cc2c(-c3ccc(C4CC4)cc3OC(F)F)nnc(N[C@@H]3CCCN(C)C3)n2c1]

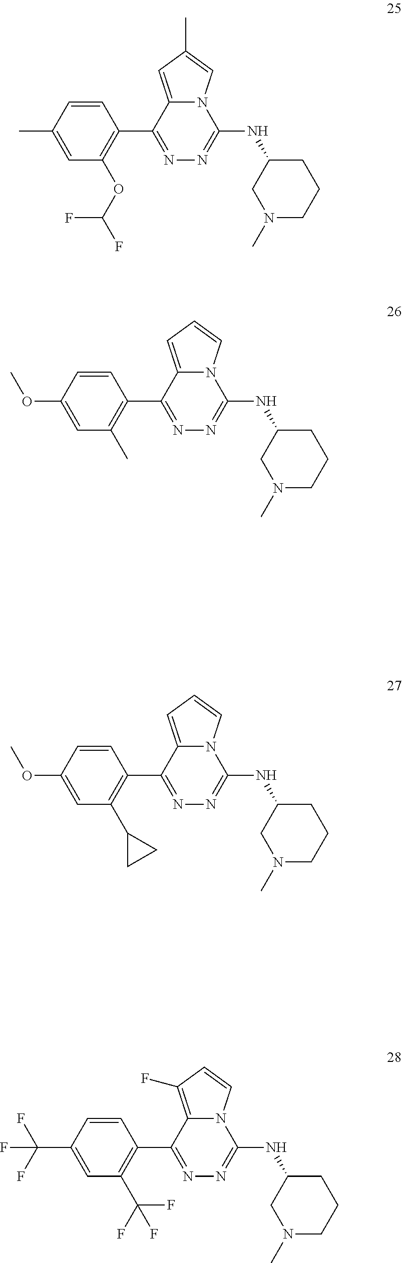 [SMILES: Cc1ccc(-c2nnc(N[C@@H]3CCCN(C)C3)n3cc(C)cc23)c(OC(F)F)c1 ; COc1ccc(-c2nnc(N[C@@H]3CCCN(C)C3)n3cccc23)c(C)c1 ; COc1ccc(-c2nnc(N[C@@H]3CCCN(C)C3)n3cccc23)c(C2CC2)c1 ; CN1CCC[C@@H](Nc2nnc(-c3ccc(C(F)(F)F)cc3C(F)(F)F)c3c(F)ccn23)C1]

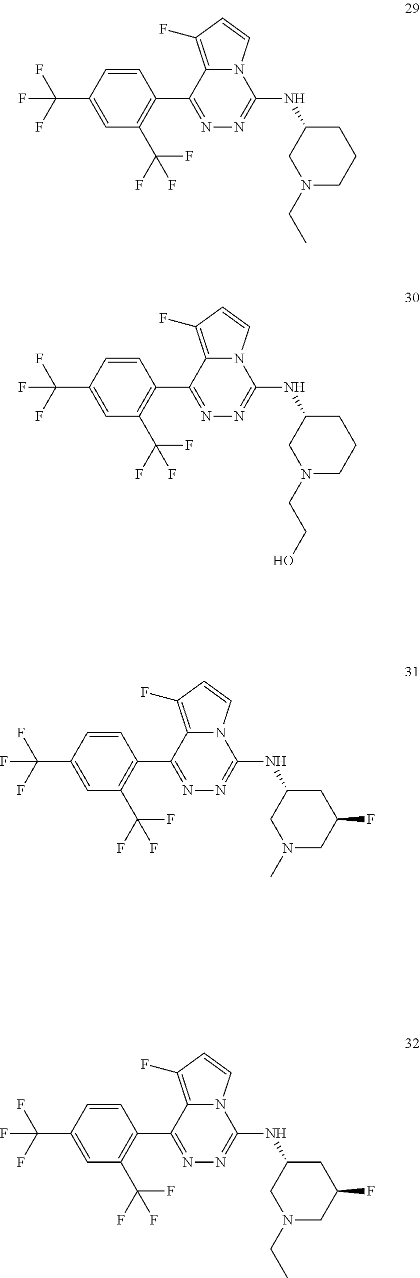 [SMILES: CCN1CCC[C@@H](Nc2nnc(-c3ccc(C(F)(F)F)cc3C(F)(F)F)c3c(F)ccn23)C1 ; OCCN1CCC[C@@H](Nc2nnc(-c3ccc(C(F)(F)F)cc3C(F)(F)F)c3c(F)ccn23)C1 ; CN1C[C@H](F)C[C@@H](Nc2nnc(-c3ccc(C(F)(F)F)cc3C(F)(F)F)c3c(F)ccn23)C1 ; CCN1C[C@H](F)C[C@@H](Nc2nnc(-c3ccc(C(F)(F)F)cc3C(F)(F)F)c3c(F)ccn23)C1]

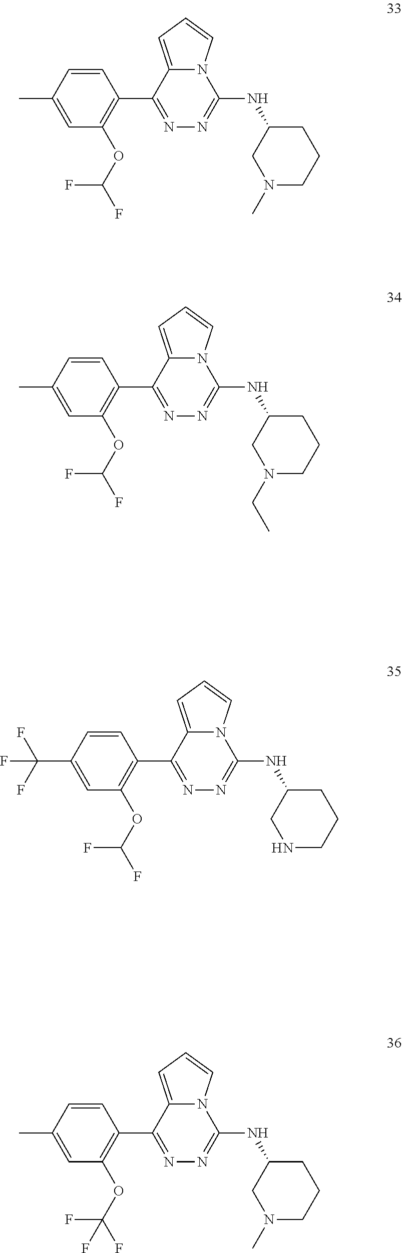 [SMILES: Cc1ccc(-c2nnc(N[C@@H]3CCCN(C)C3)n3cccc23)c(OC(F)F)c1 ; CCN1CCC[C@@H](Nc2nnc(-c3ccc(C)cc3OC(F)F)c3cccn23)C1 ; FC(F)Oc1cc(C(F)(F)F)ccc1-c1nnc(N[C@@H]2CCCNC2)n2cccc12 ; Cc1ccc(-c2nnc(N[C@@H]3CCCN(C)C3)n3cccc23)c(OC(F)(F)F)c1]

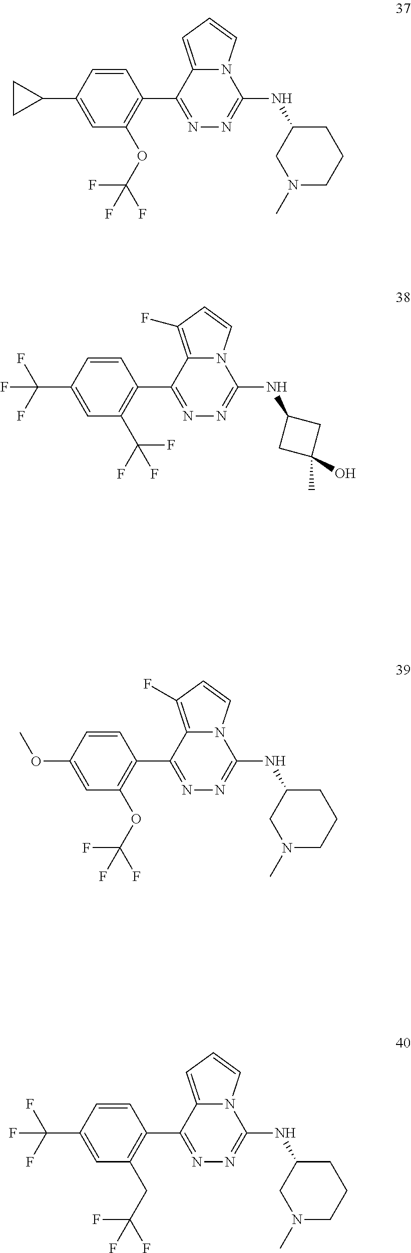 [SMILES: CN1CCC[C@@H](Nc2nnc(-c3ccc(C4CC4)cc3OC(F)(F)F)c3cccn23)C1 ; C[C@]1(O)C[C@@H](Nc2nnc(-c3ccc(C(F)(F)F)cc3C(F)(F)F)c3c(F)ccn23)C1 ; COc1ccc(-c2nnc(N[C@@H]3CCCN(C)C3)n3ccc(F)c23)c(OC(F)(F)F)c1 ; CN1CCC[C@@H](Nc2nnc(-c3ccc(C(F)(F)F)cc3CC(F)(F)F)c3cccn23)C1]

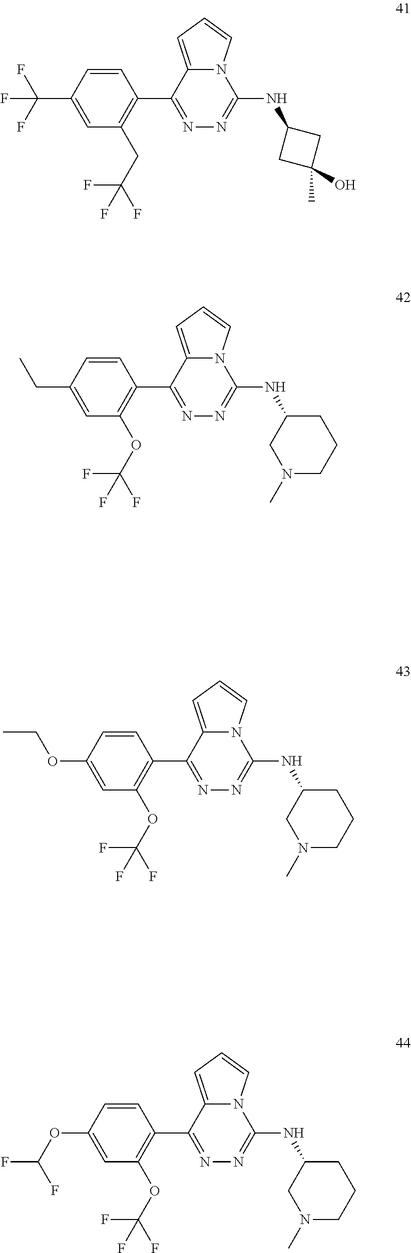 [SMILES: C[C@]1(O)C[C@@H](Nc2nnc(-c3ccc(C(F)(F)F)cc3CC(F)(F)F)c3cccn23)C1 ; CCc1ccc(-c2nnc(N[C@@H]3CCCN(C)C3)n3cccc23)c(OC(F)(F)F)c1 ; CCOc1ccc(-c2nnc(N[C@@H]3CCCN(C)C3)n3cccc23)c(OC(F)(F)F)c1 ; CN1CCC[C@@H](Nc2nnc(-c3ccc(OC(F)F)cc3OC(F)(F)F)c3cccn23)C1]

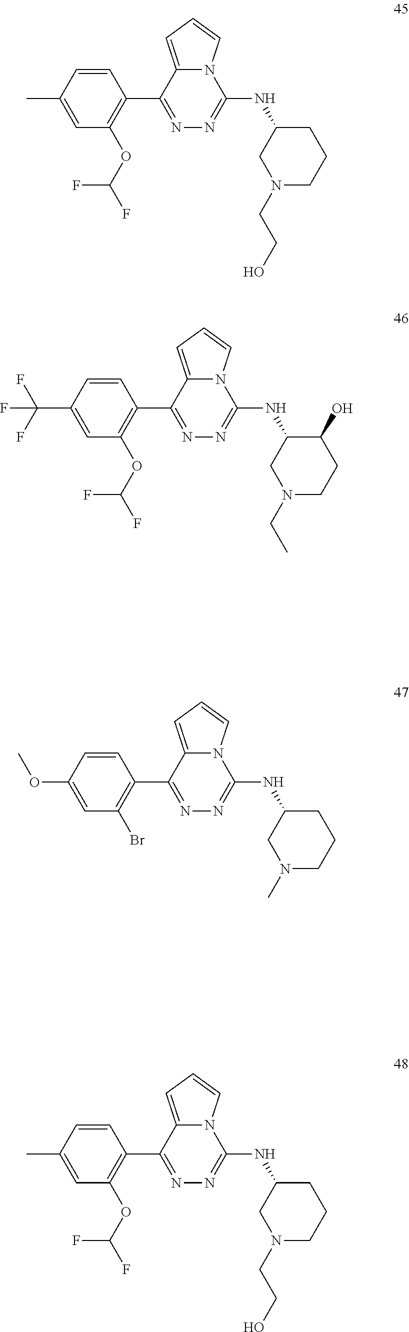 [SMILES: Cc1ccc(-c2nnc(N[C@@H]3CCCN(CCO)C3)n3cccc23)c(OC(F)F)c1 ; CCN1CC[C@H](O)[C@@H](Nc2nnc(-c3ccc(C(F)(F)F)cc3OC(F)F)c3cccn23)C1 ; COc1ccc(-c2nnc(N[C@@H]3CCCN(C)C3)n3cccc23)c(Br)c1 ; Cc1ccc(-c2nnc(N[C@@H]3CCCN(CCO)C3)n3cccc23)c(OC(F)F)c1]

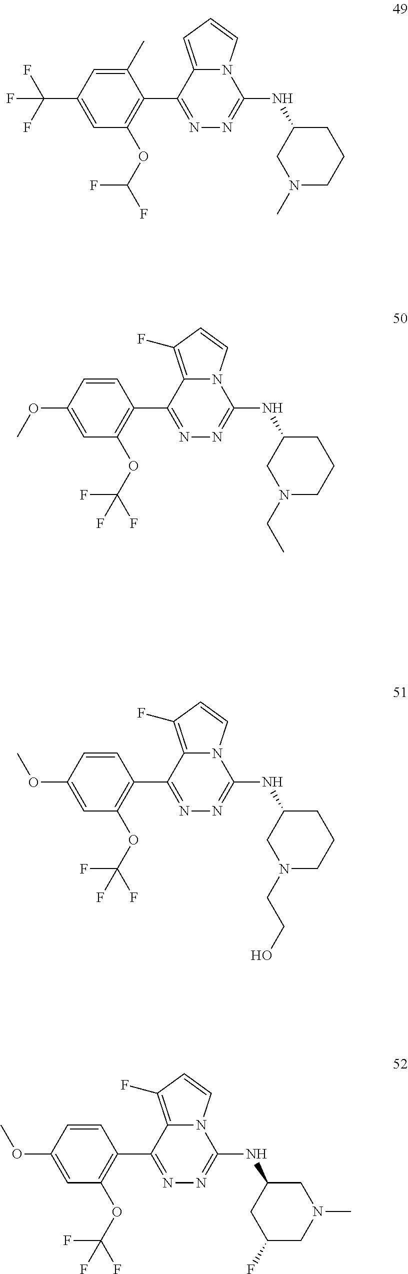

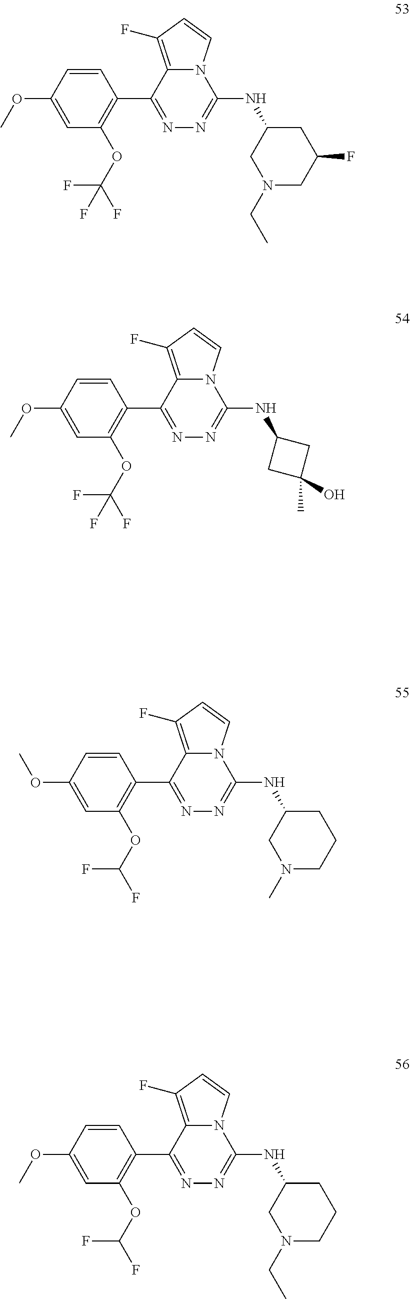 [SMILES: CCN1C[C@H](F)C[C@@H](Nc2nnc(-c3ccc(OC)cc3OC(F)(F)F)c3c(F)ccn23)C1 ; COc1ccc(-c2nnc(N[C@H]3C[C@@](C)(O)C3)n3ccc(F)c23)c(OC(F)(F)F)c1 ; COc1ccc(-c2nnc(N[C@@H]3CCCN(C)C3)n3ccc(F)c23)c(OC(F)F)c1 ; CCN1CCC[C@@H](Nc2nnc(-c3ccc(OC)cc3OC(F)F)c3c(F)ccn23)C1]

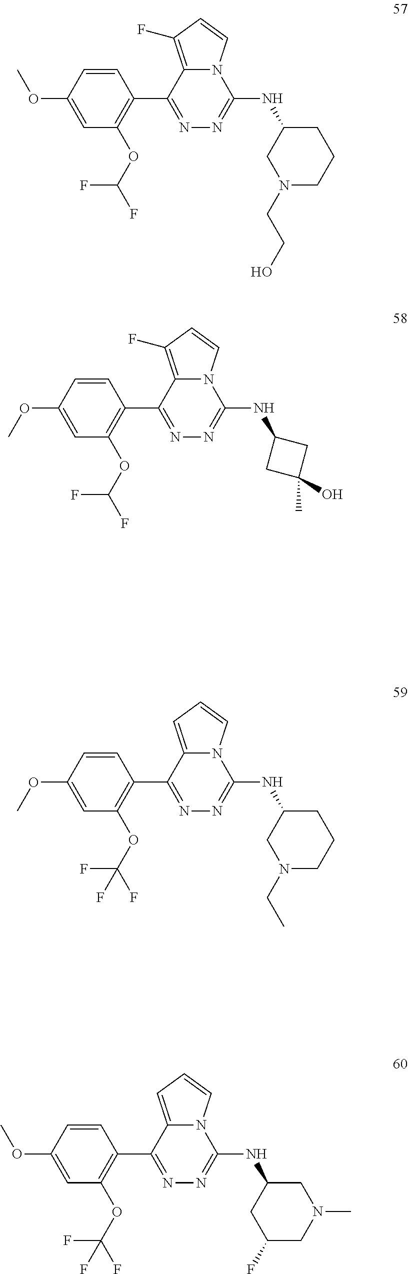

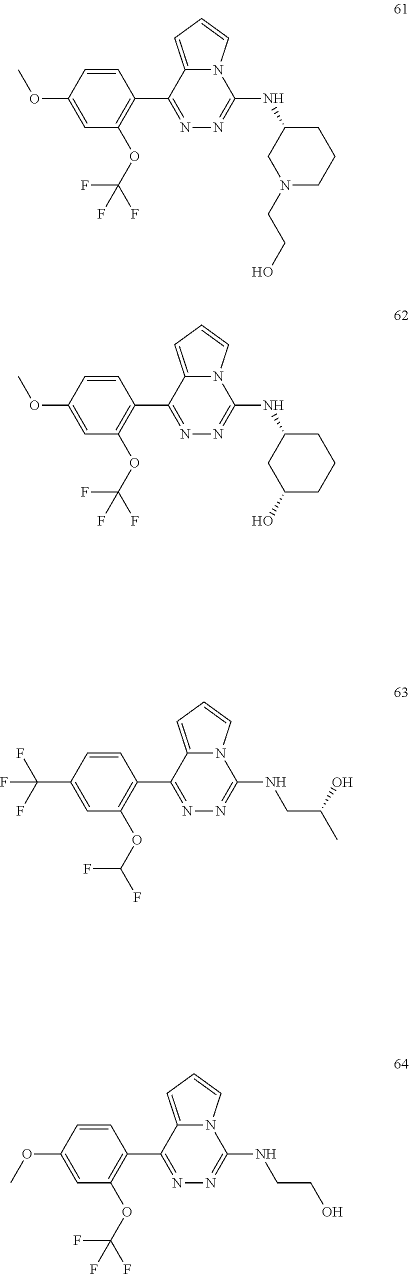 [SMILES: COc1ccc(-c2nnc(N[C@@H]3CCCN(CCO)C3)n3cccc23)c(OC(F)(F)F)c1 ; COc1ccc(-c2nnc(N[C@@H]3CCC[C@H](O)C3)n3cccc23)c(OC(F)(F)F)c1 ; C[C@@H](O)CNc1nnc(-c2ccc(C(F)(F)F)cc2OC(F)F)c2cccn12 ; COc1ccc(-c2nnc(NCCO)n3cccc23)c(OC(F)(F)F)c1]

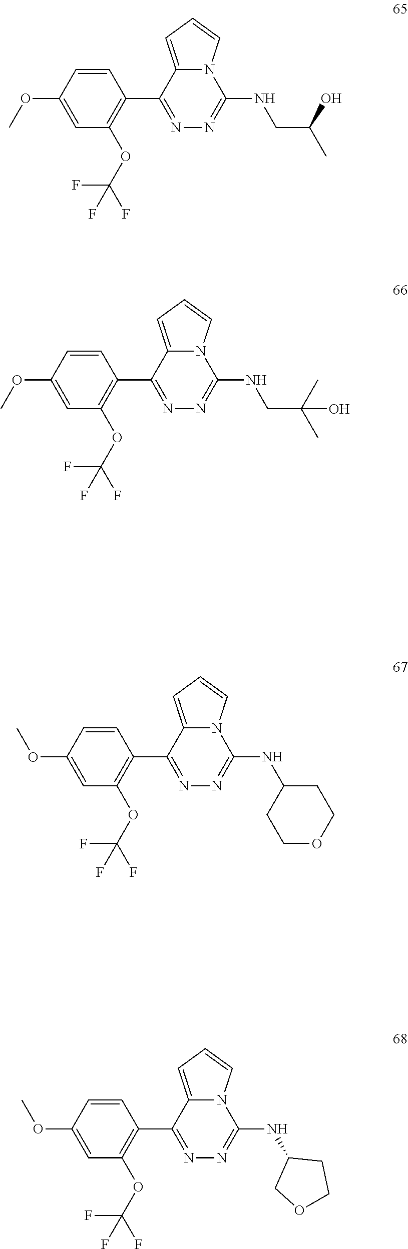 [SMILES: COc1ccc(-c2nnc(NC[C@H](C)O)n3cccc23)c(OC(F)(F)F)c1 ; COc1ccc(-c2nnc(NCC(C)(C)O)n3cccc23)c(OC(F)(F)F)c1 ; COc1ccc(-c2nnc(NC3CCOCC3)n3cccc23)c(OC(F)(F)F)c1 ; COc1ccc(-c2nnc(N[C@@H]3CCOC3)n3cccc23)c(OC(F)(F)F)c1]

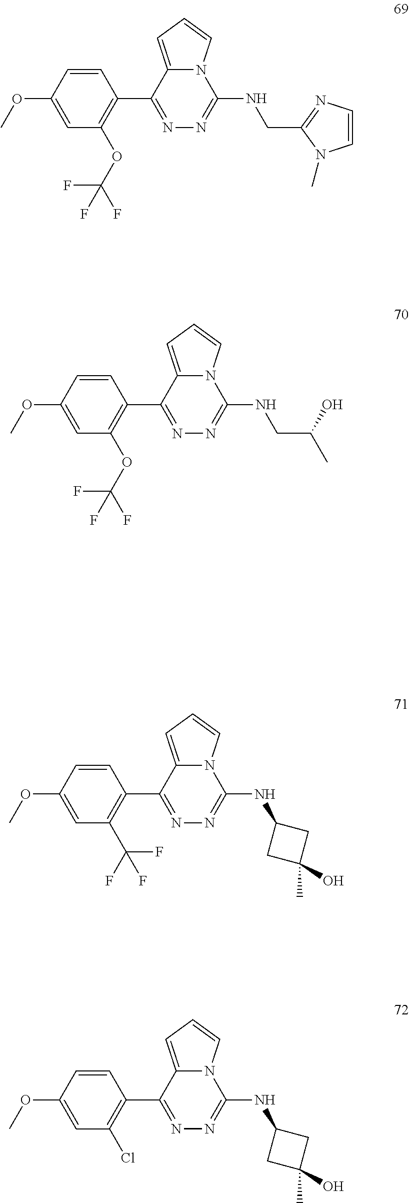 [SMILES: COc1ccc(-c2nnc(NCc3nccn3C)n3cccc23)c(OC(F)(F)F)c1 ; COc1ccc(-c2nnc(NC[C@@H](C)O)n3cccc23)c(OC(F)(F)F)c1 ; COc1ccc(-c2nnc(N[C@H]3C[C@@](C)(O)C3)n3cccc23)c(C(F)(F)F)c1 ; COc1ccc(-c2nnc(N[C@H]3C[C@@](C)(O)C3)n3cccc23)c(Cl)c1]

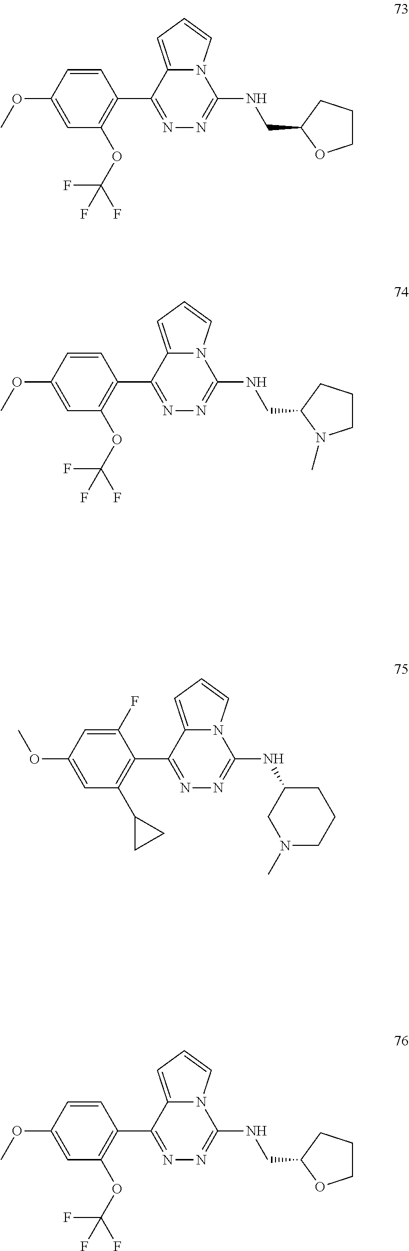 [SMILES: COc1ccc(-c2nnc(NC[C@H]3CCCO3)n3cccc23)c(OC(F)(F)F)c1 ; COc1ccc(-c2nnc(NC[C@@H]3CCCN3C)n3cccc23)c(OC(F)(F)F)c1 ; COc1cc(F)c(-c2nnc(N[C@@H]3CCCN(C)C3)n3cccc23)c(C2CC2)c1 ; COc1ccc(-c2nnc(NC[C@@H]3CCCO3)n3cccc23)c(OC(F)(F)F)c1]

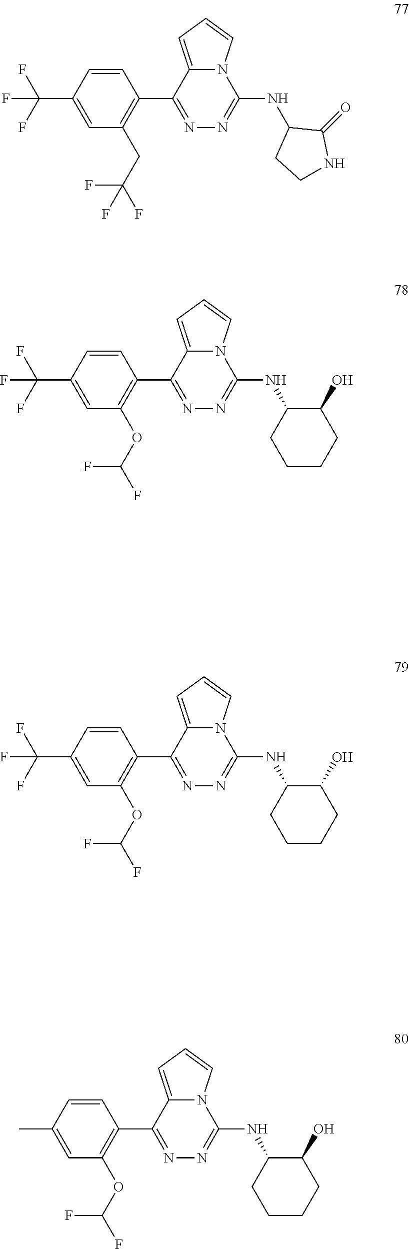 [SMILES: O=C1NCCC1Nc1nnc(-c2ccc(C(F)(F)F)cc2CC(F)(F)F)c2cccn12 ; O[C@H]1CCCC[C@@H]1Nc1nnc(-c2ccc(C(F)(F)F)cc2OC(F)F)c2cccn12 ; O[C@@H]1CCCC[C@@H]1Nc1nnc(-c2ccc(C(F)(F)F)cc2OC(F)F)c2cccn12 ; Cc1ccc(-c2nnc(N[C@H]3CCCC[C@@H]3O)n3cccc23)c(OC(F)F)c1]

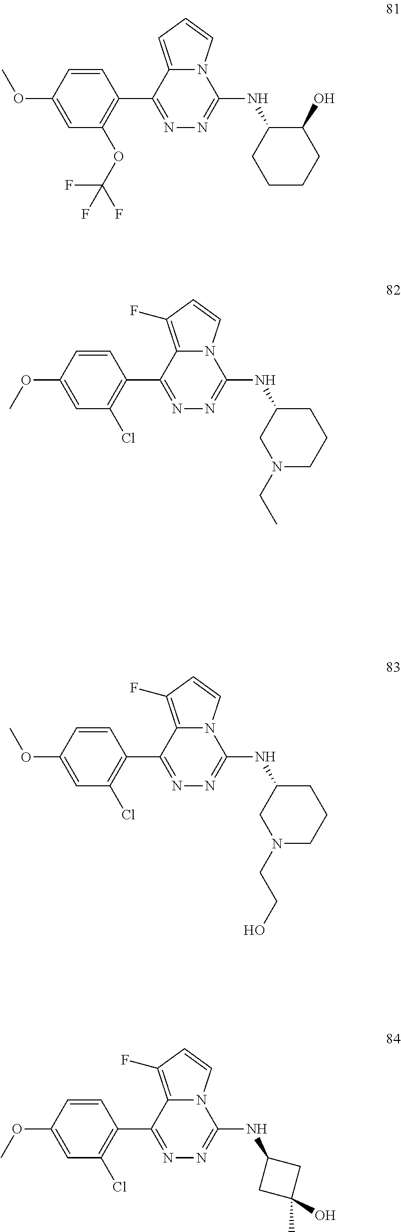 [SMILES: COc1ccc(-c2nnc(N[C@H]3CCCC[C@@H]3O)n3cccc23)c(OC(F)(F)F)c1 ; CCN1CCC[C@@H](Nc2nnc(-c3ccc(OC)cc3Cl)c3c(F)ccn23)C1 ; COc1ccc(-c2nnc(N[C@@H]3CCCN(CCO)C3)n3ccc(F)c23)c(Cl)c1 ; COc1ccc(-c2nnc(N[C@H]3C[C@@](C)(O)C3)n3ccc(F)c23)c(Cl)c1]

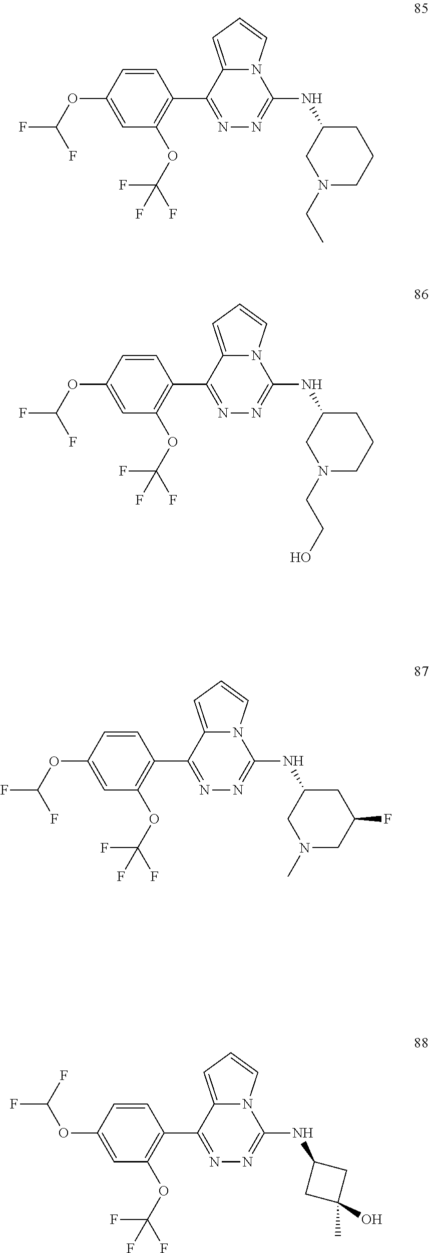 [SMILES: CCN1CCC[C@@H](Nc2nnc(-c3ccc(OC(F)F)cc3OC(F)(F)F)c3cccn23)C1 ; OCCN1CCC[C@@H](Nc2nnc(-c3ccc(OC(F)F)cc3OC(F)(F)F)c3cccn23)C1 ; CN1C[C@H](F)C[C@@H](Nc2nnc(-c3ccc(OC(F)F)cc3OC(F)(F)F)c3cccn23)C1 ; C[C@]1(O)C[C@@H](Nc2nnc(-c3ccc(OC(F)F)cc3OC(F)(F)F)c3cccn23)C1]

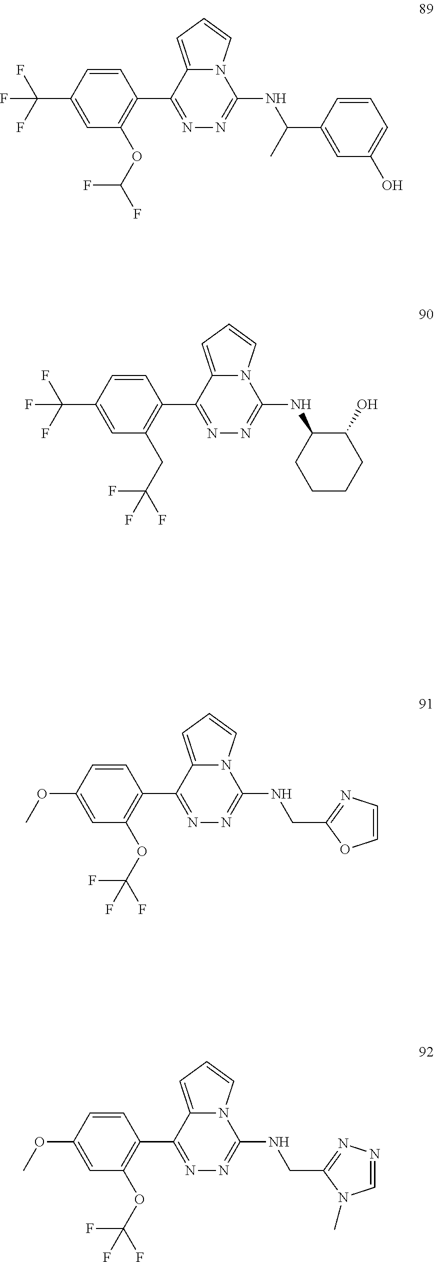 [SMILES: CC(Nc1nnc(-c2ccc(C(F)(F)F)cc2OC(F)F)c2cccn12)c1cccc(O)c1 ; O[C@@H]1CCCC[C@H]1Nc1nnc(-c2ccc(C(F)(F)F)cc2CC(F)(F)F)c2cccn12 ; COc1ccc(-c2nnc(NCc3ncco3)n3cccc23)c(OC(F)(F)F)c1 ; COc1ccc(-c2nnc(NCc3nncn3C)n3cccc23)c(OC(F)(F)F)c1]

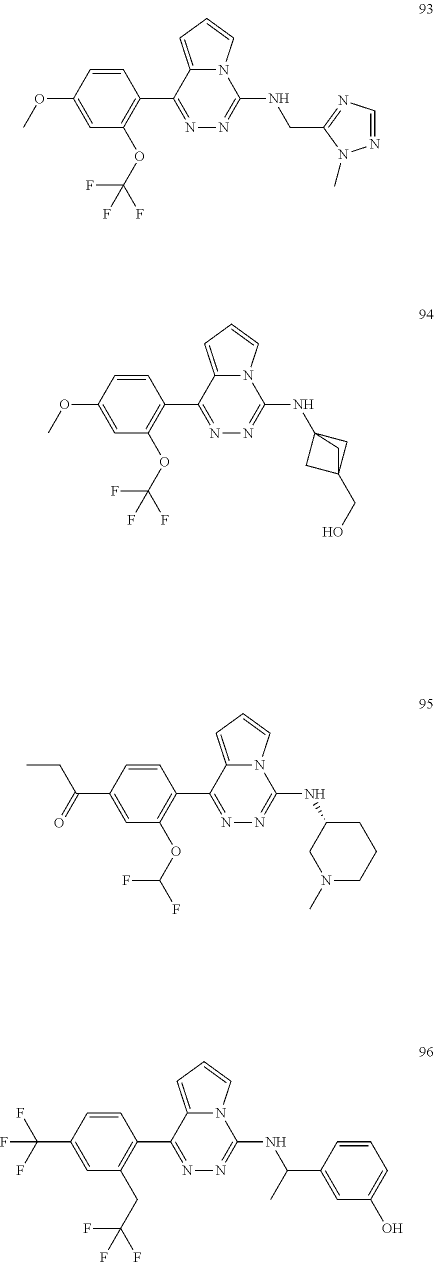 [SMILES: COc1ccc(-c2nnc(NCc3ncnn3C)n3cccc23)c(OC(F)(F)F)c1 ; COc1ccc(-c2nnc(NC34CC(CO)(C3)C4)n3cccc23)c(OC(F)(F)F)c1 ; CCC(=O)c1ccc(-c2nnc(N[C@@H]3CCCN(C)C3)n3cccc23)c(OC(F)F)c1 ; CC(Nc1nnc(-c2ccc(C(F)(F)F)cc2CC(F)(F)F)c2cccn12)c1cccc(O)c1]

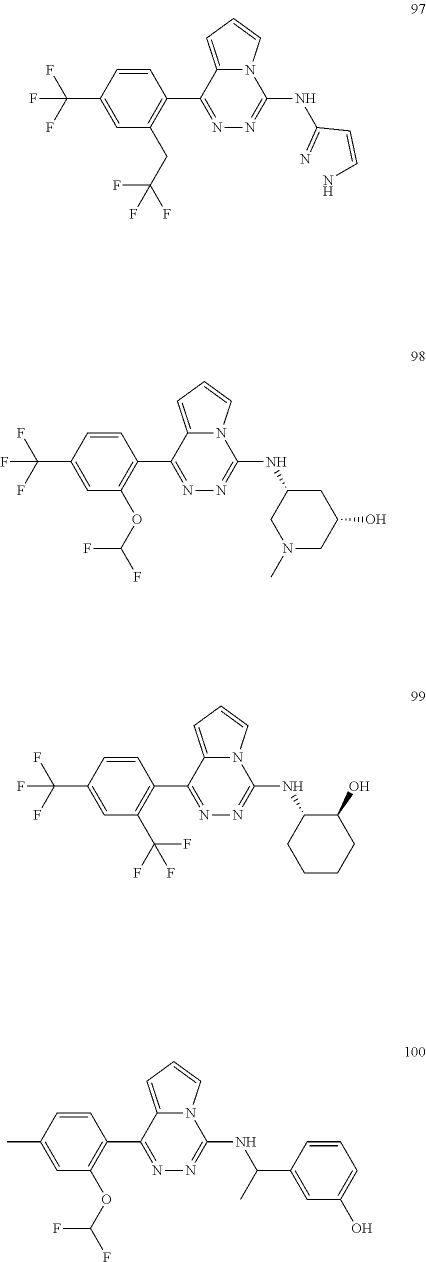 [SMILES: N=C(/C=C\N)Nc1nnc(-c2ccc(C(F)(F)F)cc2CC(F)(F)F)c2cccn12 ; CN1C[C@@H](O)C[C@@H](Nc2nnc(-c3ccc(C(F)(F)F)cc3OC(F)F)c3cccn23)C1 ; O[C@H]1CCCC[C@@H]1Nc1nnc(-c2ccc(C(F)(F)F)cc2C(F)(F)F)c2cccn12 ; Cc1ccc(-c2nnc(NC(C)c3cccc(O)c3)n3cccc23)c(OC(F)F)c1]

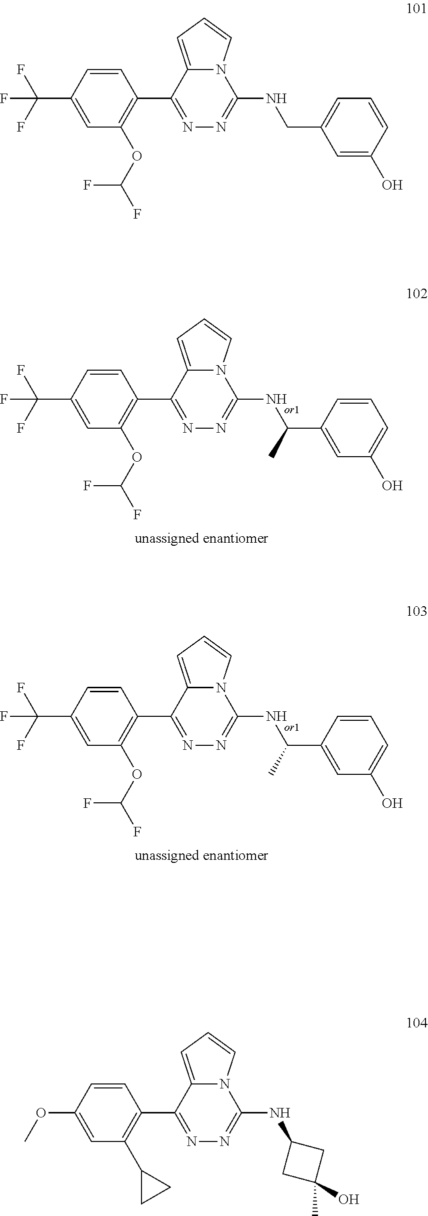 [SMILES: Oc1cccc(CNc2nnc(-c3ccc(C(F)(F)F)cc3OC(F)F)c3cccn23)c1 ; C[C@@H](Nc1nnc(-c2ccc(C(F)(F)F)cc2OC(F)F)c2cccn12)c1cccc(O)c1 ; C[C@H](Nc1nnc(-c2ccc(C(F)(F)F)cc2OC(F)F)c2cccn12)c1cccc(O)c1 ; COc1ccc(-c2nnc(N[C@H]3C[C@@](C)(O)C3)n3cccc23)c(C2CC2)c1]

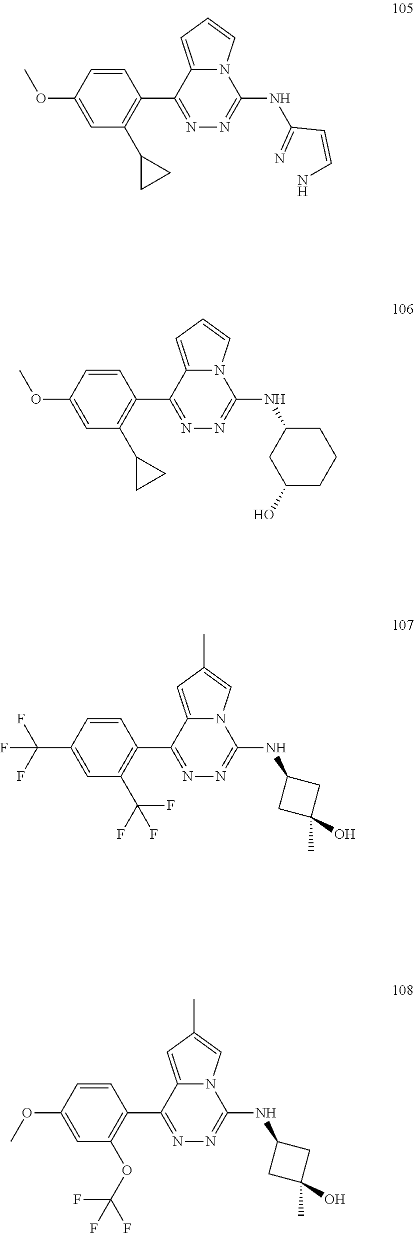 [SMILES: COc1ccc(-c2nnc(NC(=N)/C=C\N)n3cccc23)c(C2CC2)c1 ; COc1ccc(-c2nnc(N[C@@H]3CCC[C@H](O)C3)n3cccc23)c(C2CC2)c1 ; Cc1cc2c(-c3ccc(C(F)(F)F)cc3C(F)(F)F)nnc(N[C@H]3C[C@@](C)(O)C3)n2c1 ; COc1ccc(-c2nnc(N[C@H]3C[C@@](C)(O)C3)n3cc(C)cc23)c(OC(F)(F)F)c1]

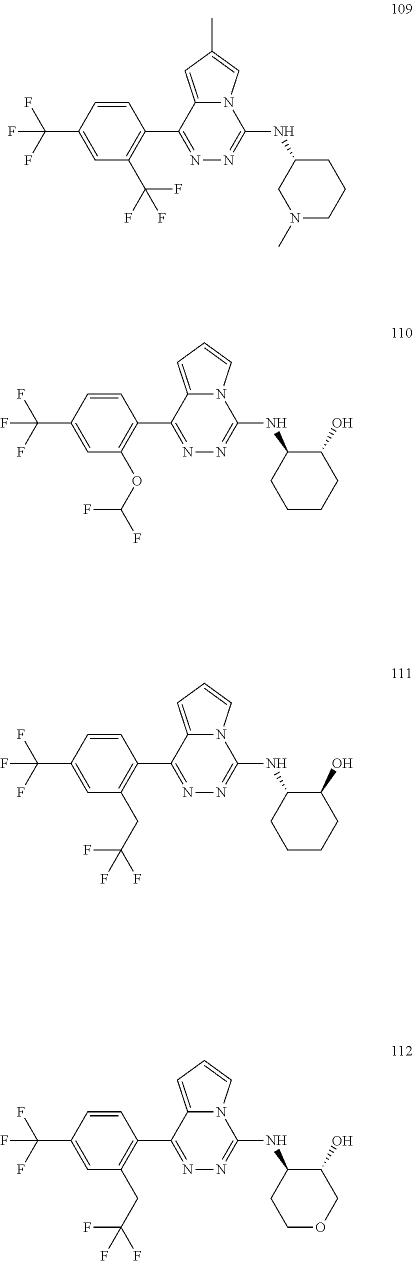 [SMILES: Cc1cc2c(-c3ccc(C(F)(F)F)cc3C(F)(F)F)nnc(N[C@@H]3CCCN(C)C3)n2c1 ; O[C@@H]1CCCC[C@H]1Nc1nnc(-c2ccc(C(F)(F)F)cc2OC(F)F)c2cccn12 ; O[C@H]1CCCC[C@@H]1Nc1nnc(-c2ccc(C(F)(F)F)cc2CC(F)(F)F)c2cccn12 ; O[C@@H]1COCC[C@H]1Nc1nnc(-c2ccc(C(F)(F)F)cc2CC(F)(F)F)c2cccn12]

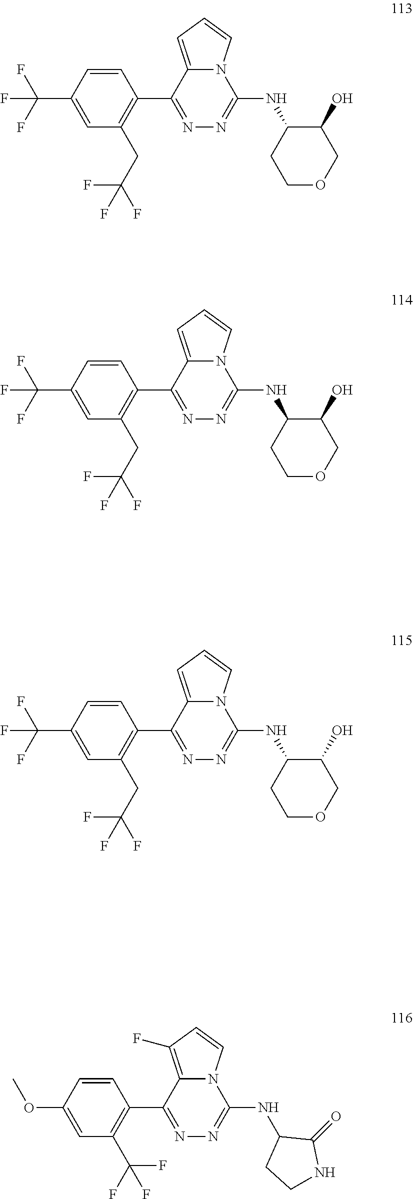 [SMILES: O[C@H]1COCC[C@@H]1Nc1nnc(-c2ccc(C(F)(F)F)cc2CC(F)(F)F)c2cccn12 ; O[C@H]1COCC[C@H]1Nc1nnc(-c2ccc(C(F)(F)F)cc2CC(F)(F)F)c2cccn12 ; O[C@@H]1COCC[C@@H]1Nc1nnc(-c2ccc(C(F)(F)F)cc2CC(F)(F)F)c2cccn12 ; COc1ccc(-c2nnc(NC3CCNC3=O)n3ccc(F)c23)c(C(F)(F)F)c1]

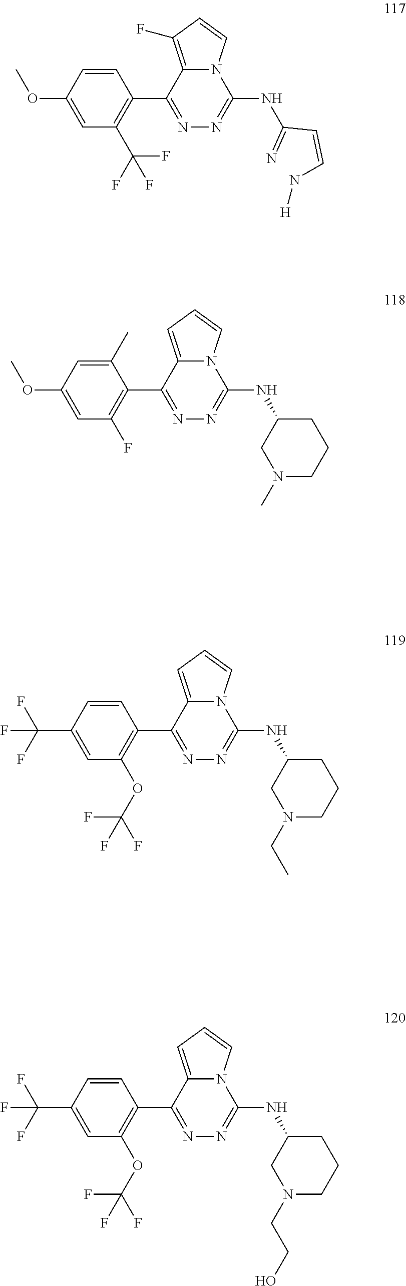 [SMILES: COc1ccc(-c2nnc(NC(=N)/C=C\N)n3ccc(F)c23)c(C(F)(F)F)c1 ; COc1cc(C)c(-c2nnc(N[C@@H]3CCCN(C)C3)n3cccc23)c(F)c1 ; CCN1CCC[C@@H](Nc2nnc(-c3ccc(C(F)(F)F)cc3OC(F)(F)F)c3cccn23)C1 ; OCCN1CCC[C@@H](Nc2nnc(-c3ccc(C(F)(F)F)cc3OC(F)(F)F)c3cccn23)C1]

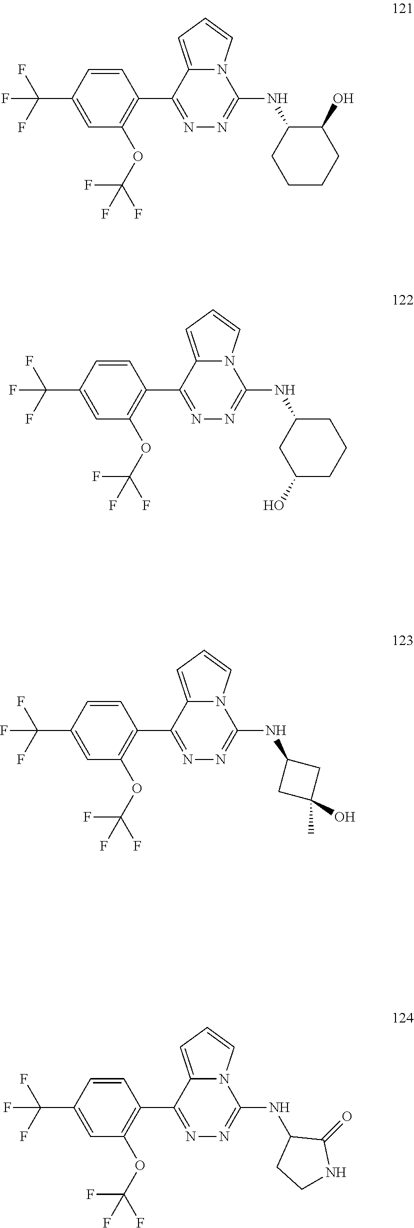 [SMILES: O[C@H]1CCCC[C@@H]1Nc1nnc(-c2ccc(C(F)(F)F)cc2OC(F)(F)F)c2cccn12 ; O[C@H]1CCC[C@@H](Nc2nnc(-c3ccc(C(F)(F)F)cc3OC(F)(F)F)c3cccn23)C1 ; C[C@]1(O)C[C@@H](Nc2nnc(-c3ccc(C(F)(F)F)cc3OC(F)(F)F)c3cccn23)C1 ; O=C1NCCC1Nc1nnc(-c2ccc(C(F)(F)F)cc2OC(F)(F)F)c2cccn12]

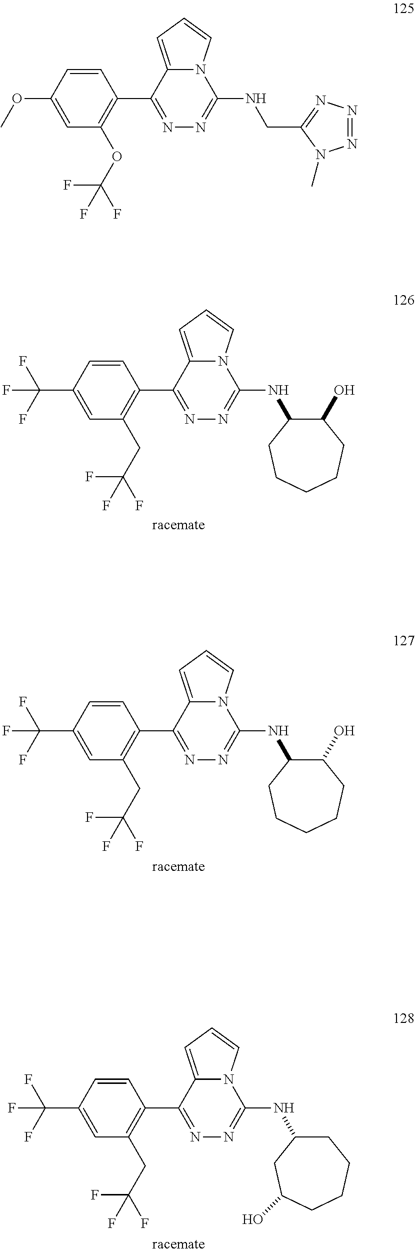

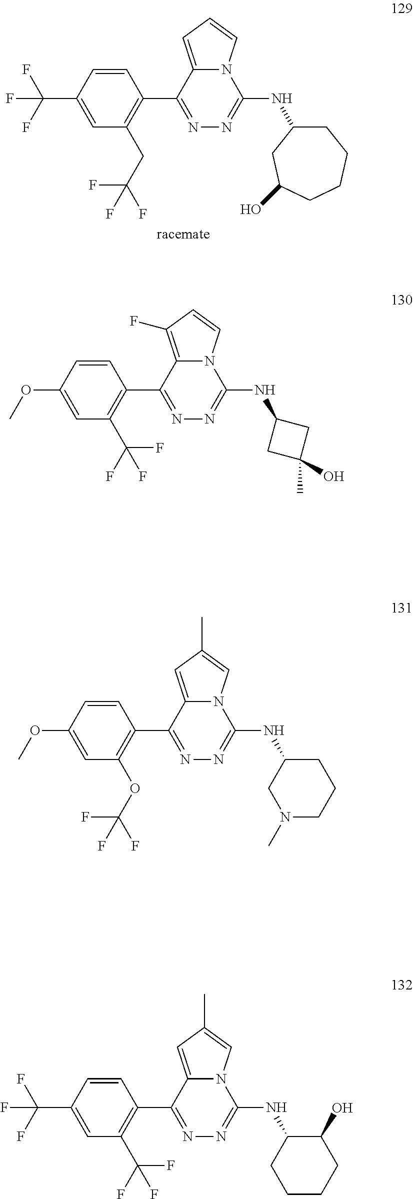

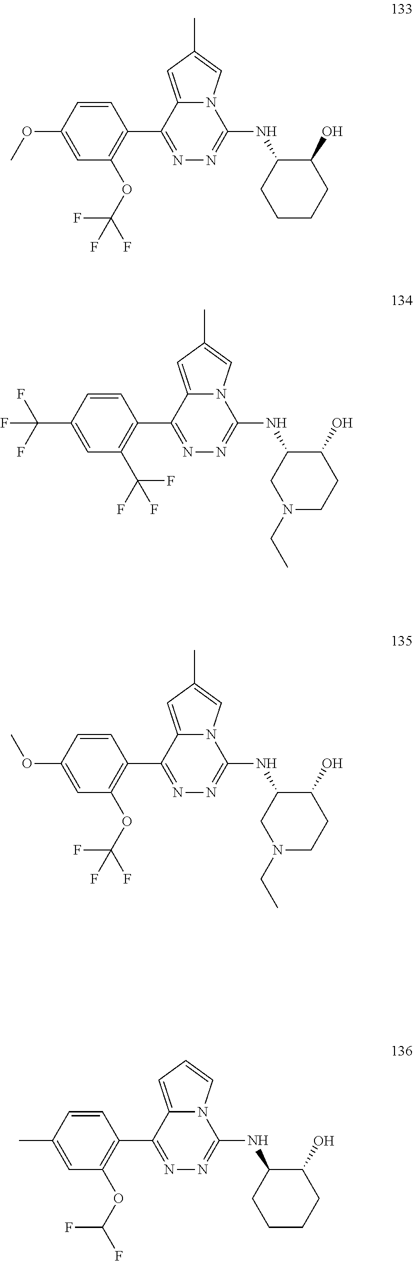 [SMILES: COc1ccc(-c2nnc(N[C@H]3CCCC[C@@H]3O)n3cc(C)cc23)c(OC(F)(F)F)c1 ; CCN1CC[C@@H](O)[C@@H](Nc2nnc(-c3ccc(C(F)(F)F)cc3C(F)(F)F)c3cc(C)cn23)C1 ; CCN1CC[C@@H](O)[C@@H](Nc2nnc(-c3ccc(OC)cc3OC(F)(F)F)c3cc(C)cn23)C1 ; Cc1ccc(-c2nnc(N[C@@H]3CCCC[C@H]3O)n3cccc23)c(OC(F)F)c1]

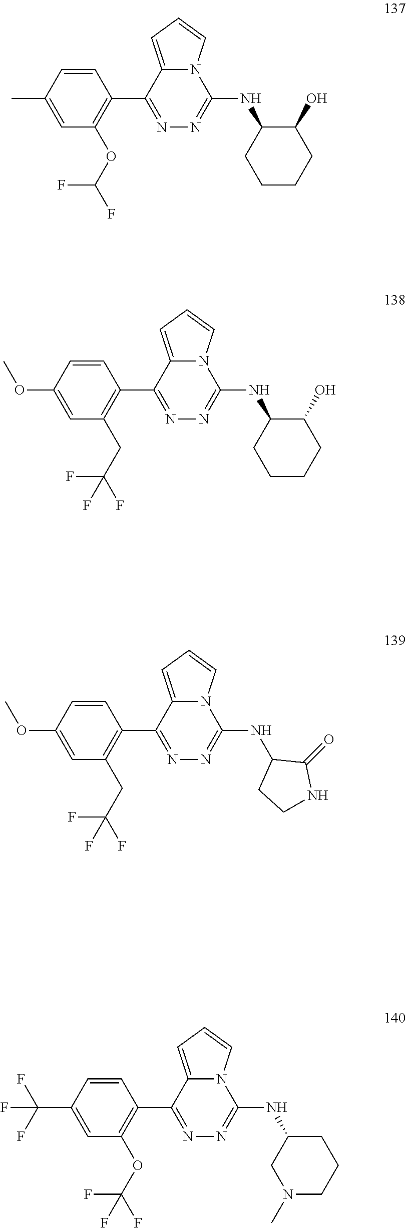 [SMILES: Cc1ccc(-c2nnc(N[C@@H]3CCCC[C@@H]3O)n3cccc23)c(OC(F)F)c1 ; COc1ccc(-c2nnc(N[C@@H]3CCCC[C@H]3O)n3cccc23)c(CC(F)(F)F)c1 ; COc1ccc(-c2nnc(NC3CCNC3=O)n3cccc23)c(CC(F)(F)F)c1 ; CN1CCC[C@@H](Nc2nnc(-c3ccc(C(F)(F)F)cc3OC(F)(F)F)c3cccn23)C1]

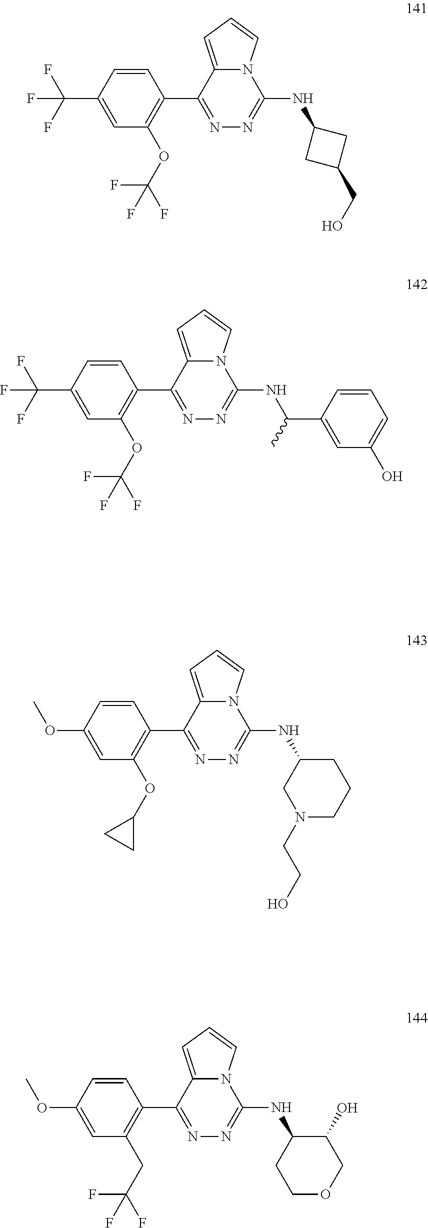 [SMILES: OC[C@H]1C[C@@H](Nc2nnc(-c3ccc(C(F)(F)F)cc3OC(F)(F)F)c3cccn23)C1 ; CC(Nc1nnc(-c2ccc(C(F)(F)F)cc2OC(F)(F)F)c2cccn12)c1cccc(O)c1 ; COc1ccc(-c2nnc(N[C@@H]3CCCN(CCO)C3)n3cccc23)c(OC2CC2)c1 ; COc1ccc(-c2nnc(N[C@@H]3CCOC[C@H]3O)n3cccc23)c(CC(F)(F)F)c1]

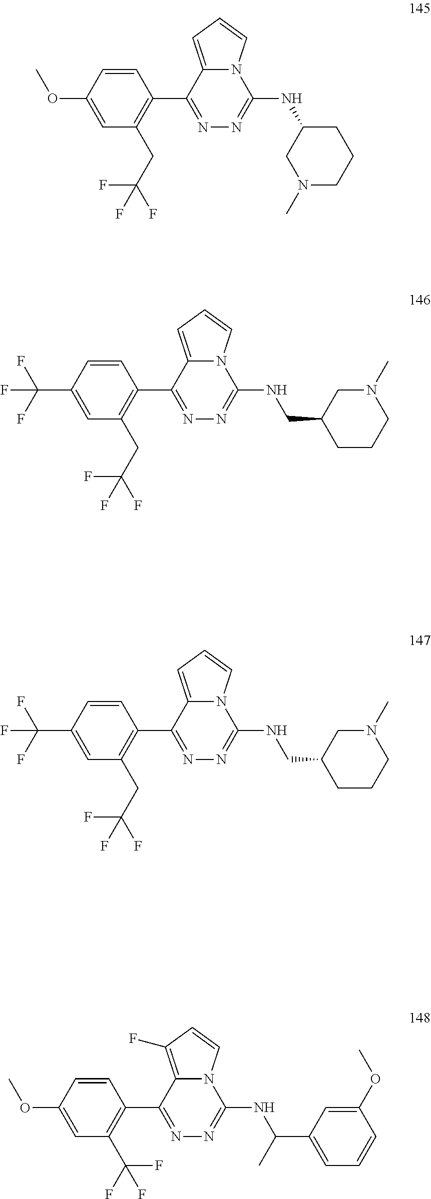

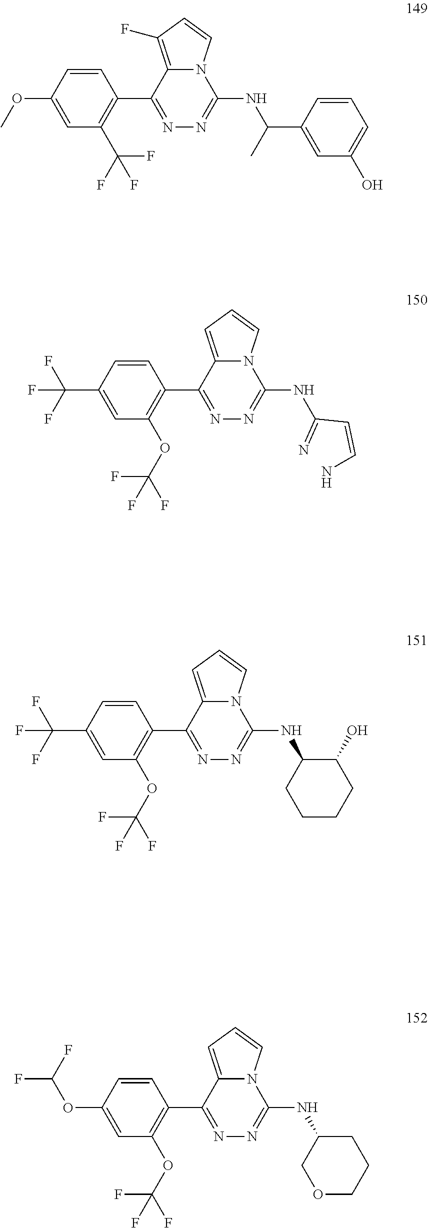 [SMILES: COc1ccc(-c2nnc(NC(C)c3cccc(O)c3)n3ccc(F)c23)c(C(F)(F)F)c1 ; N=C(/C=C\N)Nc1nnc(-c2ccc(C(F)(F)F)cc2OC(F)(F)F)c2cccn12 ; O[C@@H]1CCCC[C@H]1Nc1nnc(-c2ccc(C(F)(F)F)cc2OC(F)(F)F)c2cccn12 ; FC(F)Oc1ccc(-c2nnc(N[C@@H]3CCCOC3)n3cccc23)c(OC(F)(F)F)c1]

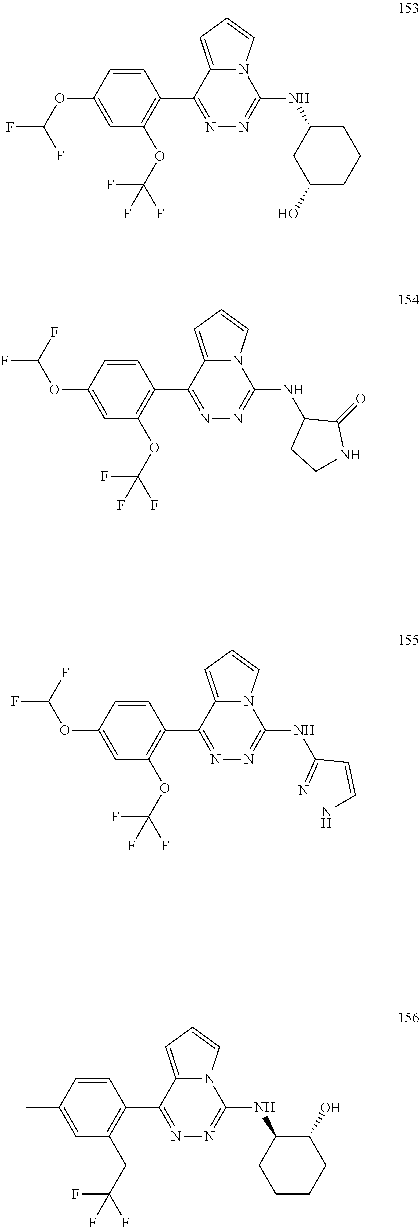 [SMILES: O[C@H]1CCC[C@@H](Nc2nnc(-c3ccc(OC(F)F)cc3OC(F)(F)F)c3cccn23)C1 ; O=C1NCCC1Nc1nnc(-c2ccc(OC(F)F)cc2OC(F)(F)F)c2cccn12 ; N=C(/C=C\N)Nc1nnc(-c2ccc(OC(F)F)cc2OC(F)(F)F)c2cccn12 ; Cc1ccc(-c2nnc(N[C@@H]3CCCC[C@H]3O)n3cccc23)c(CC(F)(F)F)c1]

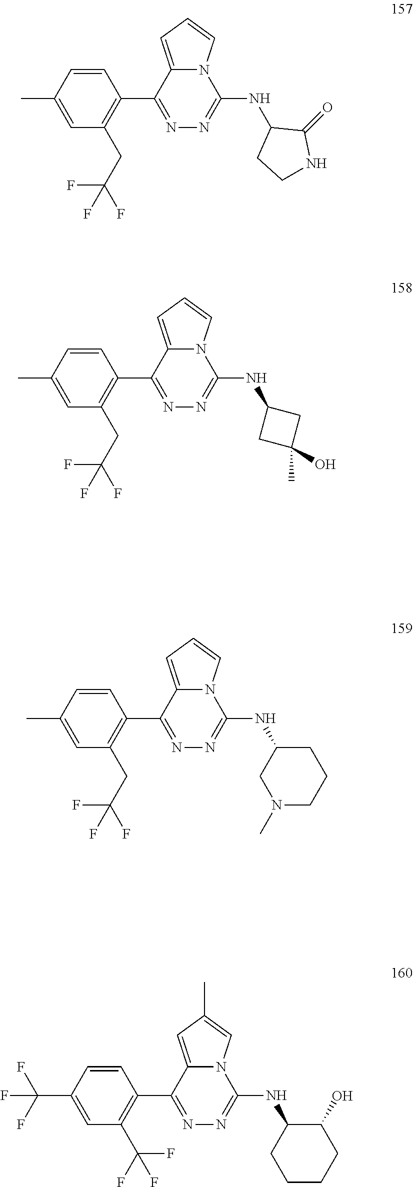 [SMILES: Cc1ccc(-c2nnc(NC3CCNC3=O)n3cccc23)c(CC(F)(F)F)c1 ; Cc1ccc(-c2nnc(N[C@H]3C[C@@](C)(O)C3)n3cccc23)c(CC(F)(F)F)c1 ; Cc1ccc(-c2nnc(N[C@@H]3CCCN(C)C3)n3cccc23)c(CC(F)(F)F)c1 ; Cc1cc2c(-c3ccc(C(F)(F)F)cc3C(F)(F)F)nnc(N[C@@H]3CCCC[C@H]3O)n2c1]

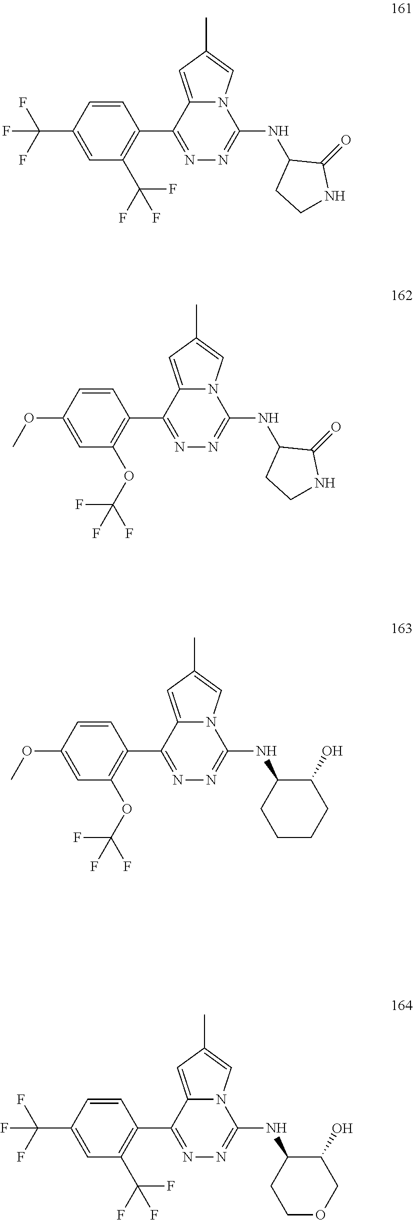 [SMILES: Cc1cc2c(-c3ccc(C(F)(F)F)cc3C(F)(F)F)nnc(NC3CCNC3=O)n2c1 ; COc1ccc(-c2nnc(NC3CCNC3=O)n3cc(C)cc23)c(OC(F)(F)F)c1 ; COc1ccc(-c2nnc(N[C@@H]3CCCC[C@H]3O)n3cc(C)cc23)c(OC(F)(F)F)c1 ; Cc1cc2c(-c3ccc(C(F)(F)F)cc3C(F)(F)F)nnc(N[C@@H]3CCOC[C@H]3O)n2c1]

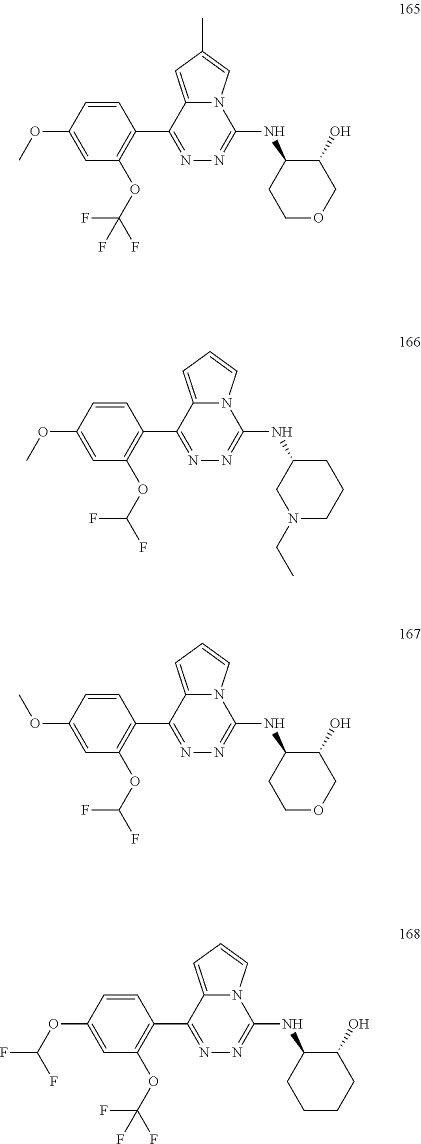 [SMILES: COc1ccc(-c2nnc(N[C@@H]3CCOC[C@H]3O)n3cc(C)cc23)c(OC(F)(F)F)c1 ; CCN1CCC[C@@H](Nc2nnc(-c3ccc(OC)cc3OC(F)F)c3cccn23)C1 ; COc1ccc(-c2nnc(N[C@@H]3CCOC[C@H]3O)n3cccc23)c(OC(F)F)c1 ; O[C@@H]1CCCC[C@H]1Nc1nnc(-c2ccc(OC(F)F)cc2OC(F)(F)F)c2cccn12]

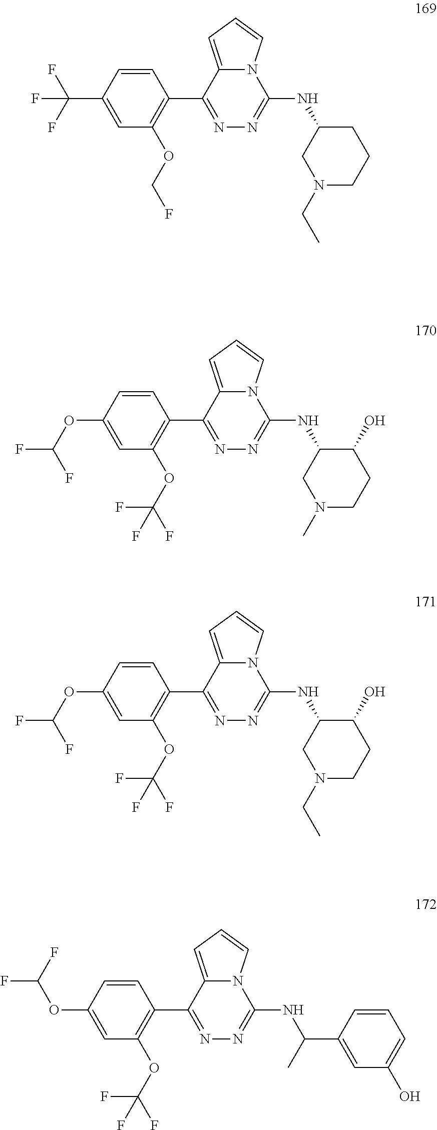 [SMILES: CCN1CCC[C@@H](Nc2nnc(-c3ccc(C(F)(F)F)cc3OCF)c3cccn23)C1 ; CN1CC[C@@H](O)[C@@H](Nc2nnc(-c3ccc(OC(F)F)cc3OC(F)(F)F)c3cccn23)C1 ; CCN1CC[C@@H](O)[C@@H](Nc2nnc(-c3ccc(OC(F)F)cc3OC(F)(F)F)c3cccn23)C1 ; CC(Nc1nnc(-c2ccc(OC(F)F)cc2OC(F)(F)F)c2cccn12)c1cccc(O)c1]

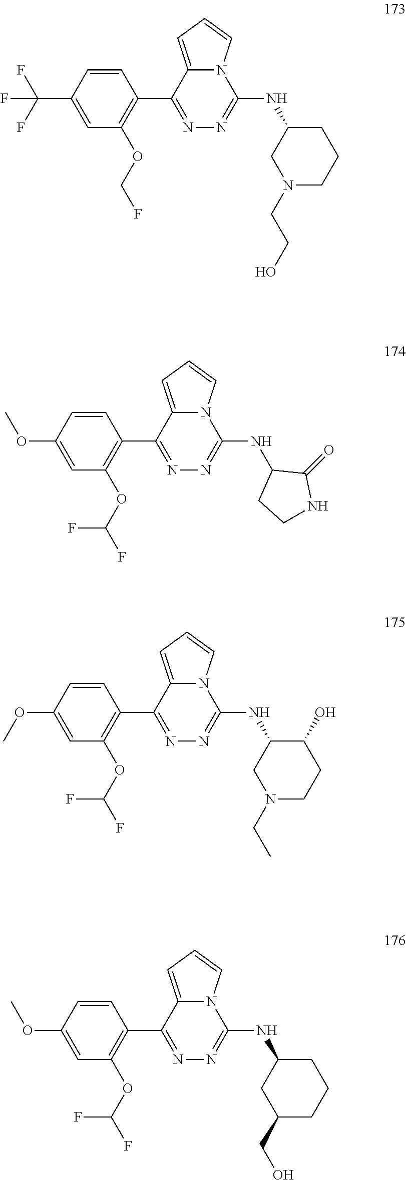 [SMILES: OCCN1CCC[C@@H](Nc2nnc(-c3ccc(C(F)(F)F)cc3OCF)c3cccn23)C1 ; COc1ccc(-c2nnc(NC3CCNC3=O)n3cccc23)c(OC(F)F)c1 ; CCN1CC[C@@H](O)[C@@H](Nc2nnc(-c3ccc(OC)cc3OC(F)F)c3cccn23)C1 ; COc1ccc(-c2nnc(N[C@H]3CCC[C@@H](CO)C3)n3cccc23)c(OC(F)F)c1]

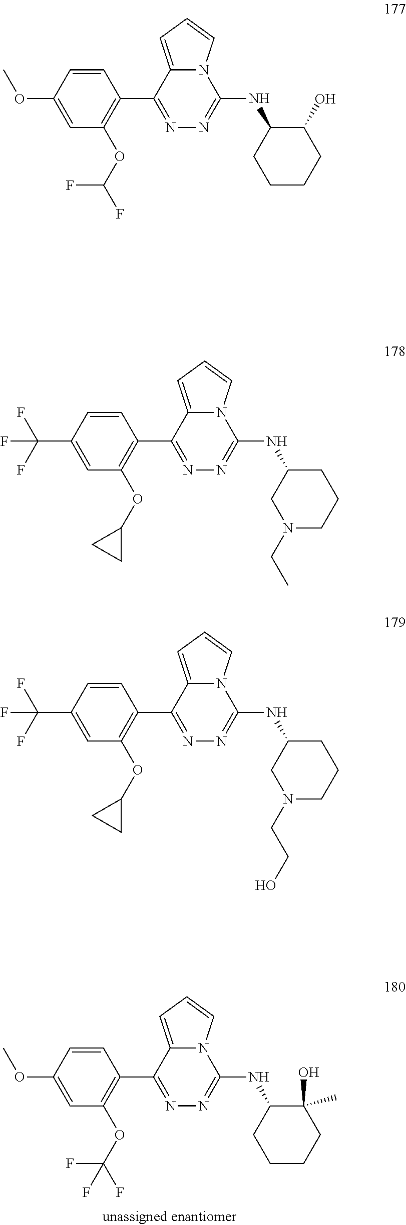

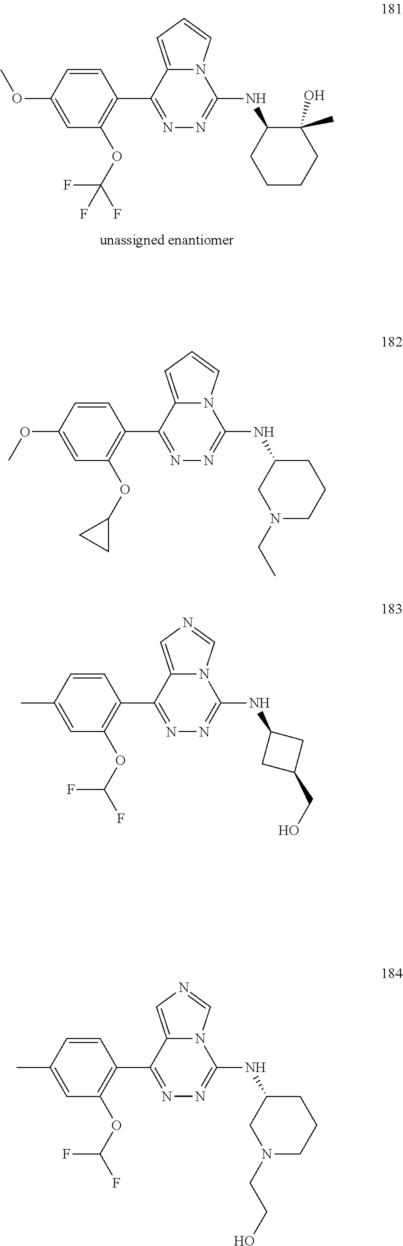

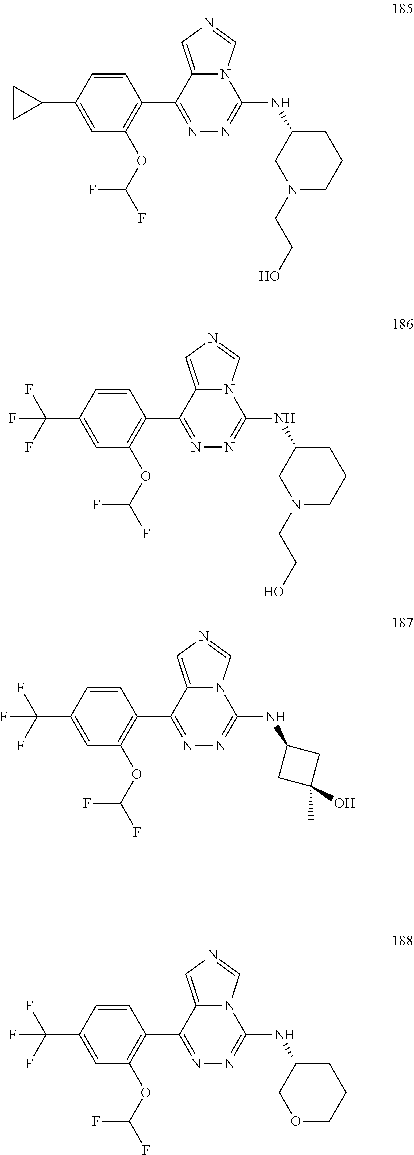 [SMILES: OCCN1CCC[C@@H](Nc2nnc(-c3ccc(C4CC4)cc3OC(F)F)c3cncn23)C1 ; OCCN1CCC[C@@H](Nc2nnc(-c3ccc(C(F)(F)F)cc3OC(F)F)c3cncn23)C1 ; C[C@]1(O)C[C@@H](Nc2nnc(-c3ccc(C(F)(F)F)cc3OC(F)F)c3cncn23)C1 ; FC(F)Oc1cc(C(F)(F)F)ccc1-c1nnc(N[C@@H]2CCCOC2)n2cncc12]

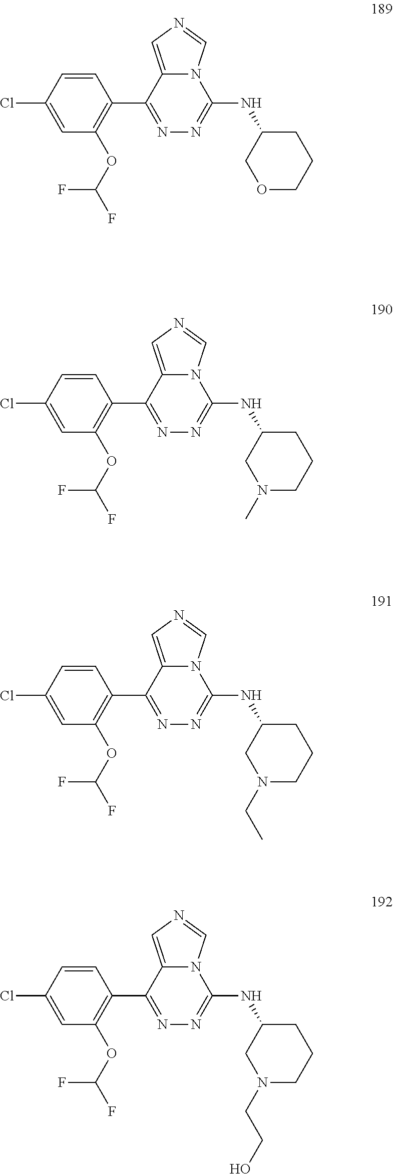 [SMILES: FC(F)Oc1cc(Cl)ccc1-c1nnc(N[C@@H]2CCCOC2)n2cncc12 ; CN1CCC[C@@H](Nc2nnc(-c3ccc(Cl)cc3OC(F)F)c3cncn23)C1 ; CCN1CCC[C@@H](Nc2nnc(-c3ccc(Cl)cc3OC(F)F)c3cncn23)C1 ; OCCN1CCC[C@@H](Nc2nnc(-c3ccc(Cl)cc3OC(F)F)c3cncn23)C1]

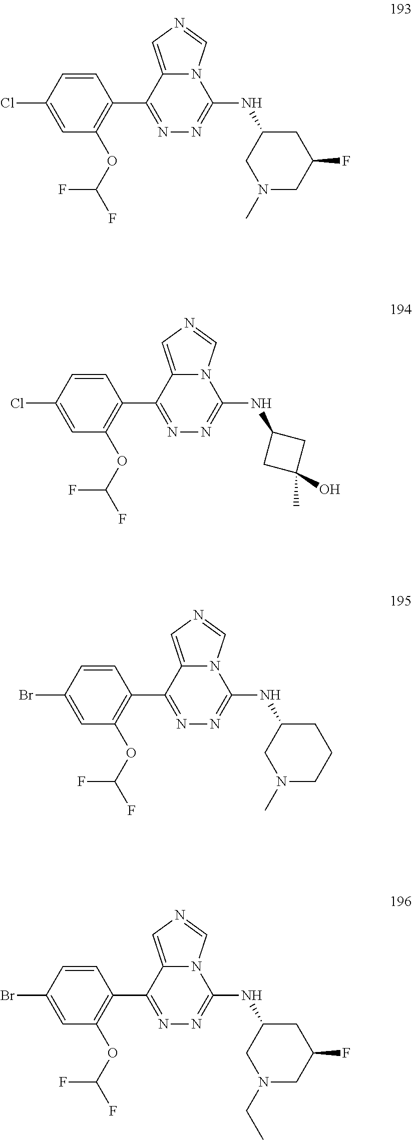 [SMILES: CN1C[C@H](F)C[C@@H](Nc2nnc(-c3ccc(Cl)cc3OC(F)F)c3cncn23)C1 ; C[C@]1(O)C[C@@H](Nc2nnc(-c3ccc(Cl)cc3OC(F)F)c3cncn23)C1 ; CN1CCC[C@@H](Nc2nnc(-c3ccc(Br)cc3OC(F)F)c3cncn23)C1 ; CCN1C[C@H](F)C[C@@H](Nc2nnc(-c3ccc(Br)cc3OC(F)F)c3cncn23)C1]

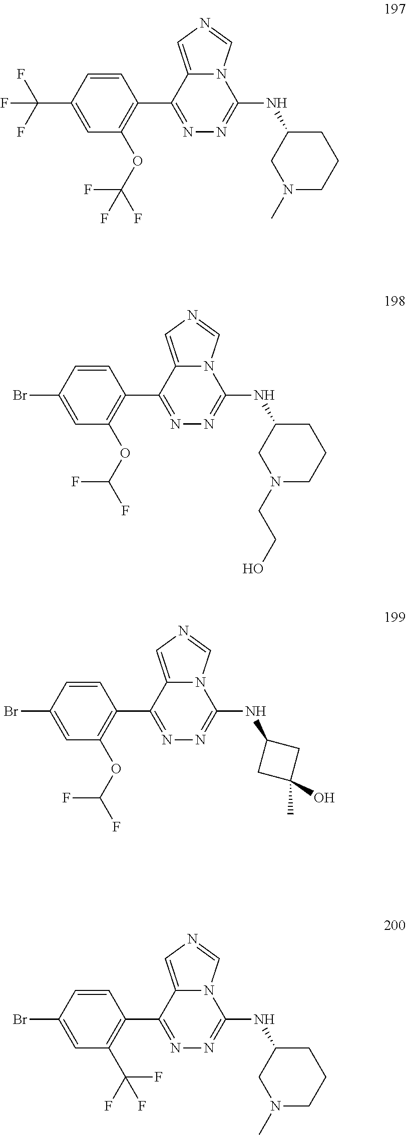 [SMILES: CN1CCC[C@@H](Nc2nnc(-c3ccc(C(F)(F)F)cc3OC(F)(F)F)c3cncn23)C1 ; OCCN1CCC[C@@H](Nc2nnc(-c3ccc(Br)cc3OC(F)F)c3cncn23)C1 ; C[C@]1(O)C[C@@H](Nc2nnc(-c3ccc(Br)cc3OC(F)F)c3cncn23)C1 ; CN1CCC[C@@H](Nc2nnc(-c3ccc(Br)cc3C(F)(F)F)c3cncn23)C1]

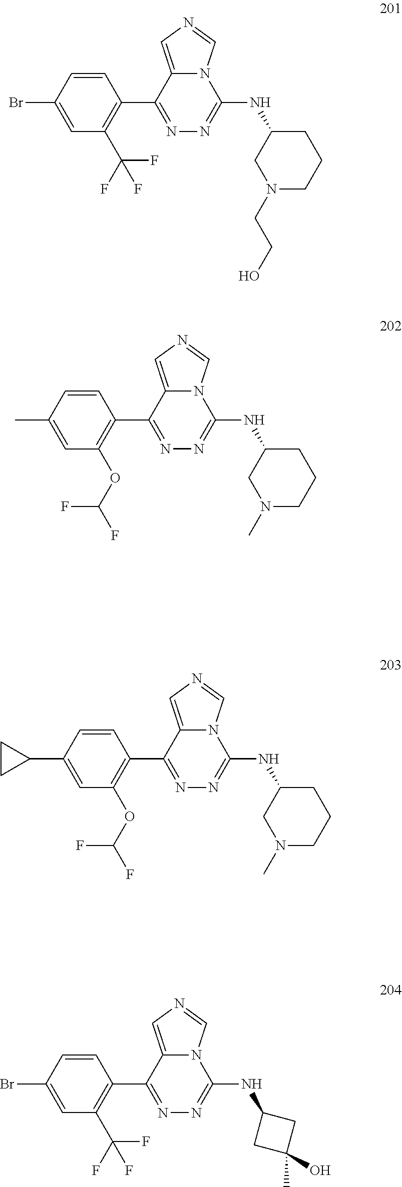 [SMILES: OCCN1CCC[C@@H](Nc2nnc(-c3ccc(Br)cc3C(F)(F)F)c3cncn23)C1 ; Cc1ccc(-c2nnc(N[C@@H]3CCCN(C)C3)n3cncc23)c(OC(F)F)c1 ; CN1CCC[C@@H](Nc2nnc(-c3ccc(C4CC4)cc3OC(F)F)c3cncn23)C1 ; C[C@]1(O)C[C@@H](Nc2nnc(-c3ccc(Br)cc3C(F)(F)F)c3cncn23)C1]

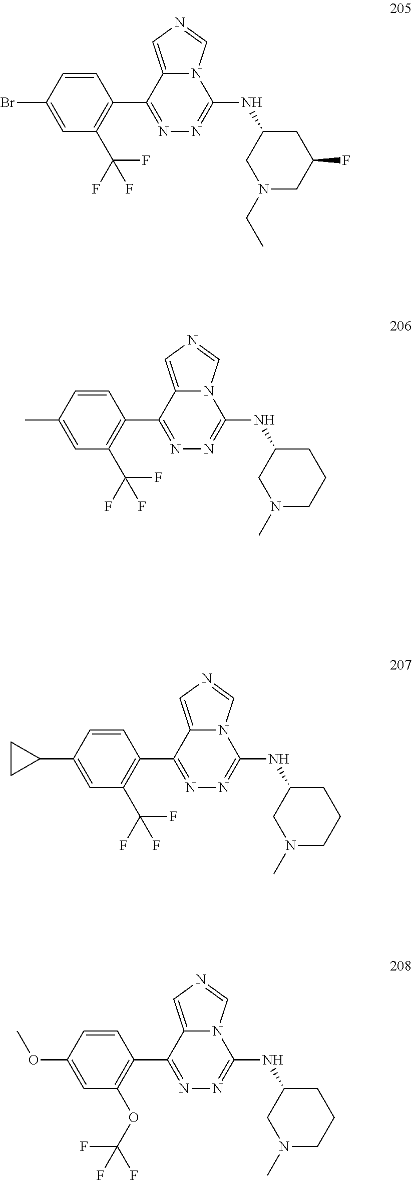 [SMILES: CCN1C[C@H](F)C[C@@H](Nc2nnc(-c3ccc(Br)cc3C(F)(F)F)c3cncn23)C1 ; Cc1ccc(-c2nnc(N[C@@H]3CCCN(C)C3)n3cncc23)c(C(F)(F)F)c1 ; CN1CCC[C@@H](Nc2nnc(-c3ccc(C4CC4)cc3C(F)(F)F)c3cncn23)C1 ; COc1ccc(-c2nnc(N[C@@H]3CCCN(C)C3)n3cncc23)c(OC(F)(F)F)c1]

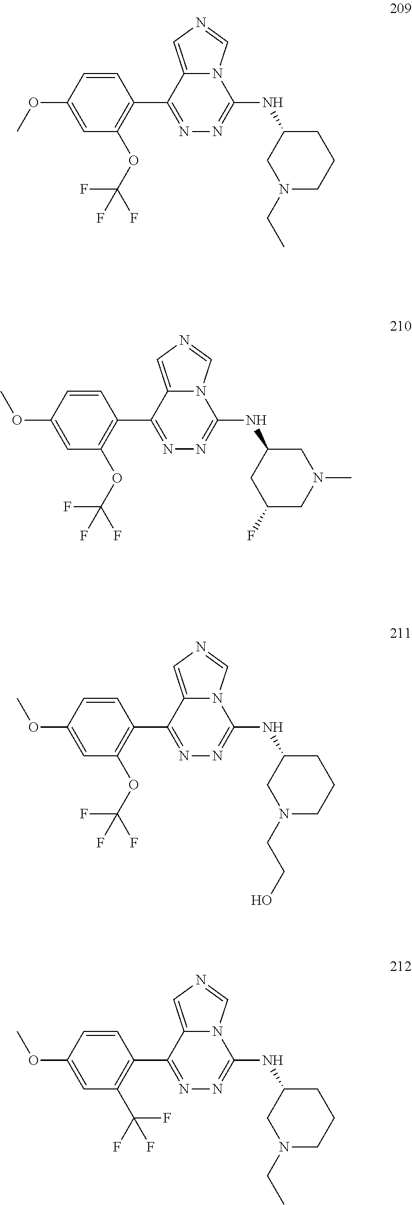

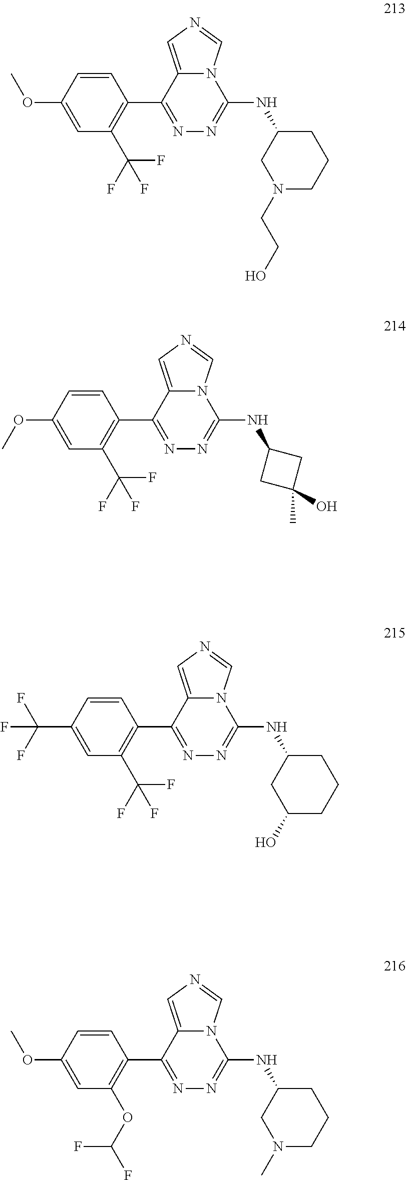 [SMILES: COc1ccc(-c2nnc(N[C@@H]3CCCN(CCO)C3)n3cncc23)c(C(F)(F)F)c1 ; COc1ccc(-c2nnc(N[C@H]3C[C@@](C)(O)C3)n3cncc23)c(C(F)(F)F)c1 ; O[C@H]1CCC[C@@H](Nc2nnc(-c3ccc(C(F)(F)F)cc3C(F)(F)F)c3cncn23)C1 ; COc1ccc(-c2nnc(N[C@@H]3CCCN(C)C3)n3cncc23)c(OC(F)F)c1]

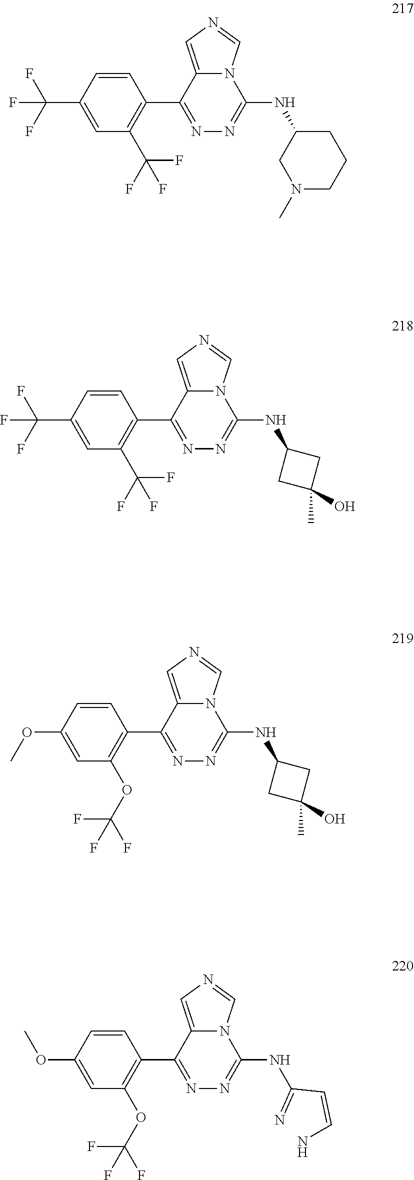 [SMILES: CN1CCC[C@@H](Nc2nnc(-c3ccc(C(F)(F)F)cc3C(F)(F)F)c3cncn23)C1 ; C[C@]1(O)C[C@@H](Nc2nnc(-c3ccc(C(F)(F)F)cc3C(F)(F)F)c3cncn23)C1 ; COc1ccc(-c2nnc(N[C@H]3C[C@@](C)(O)C3)n3cncc23)c(OC(F)(F)F)c1 ; COc1ccc(-c2nnc(NC(=N)/C=C\N)n3cncc23)c(OC(F)(F)F)c1]

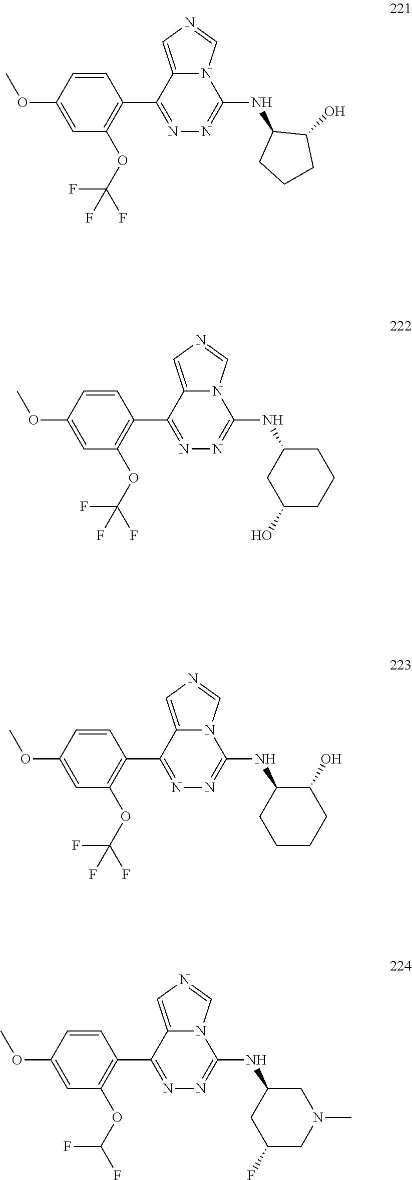

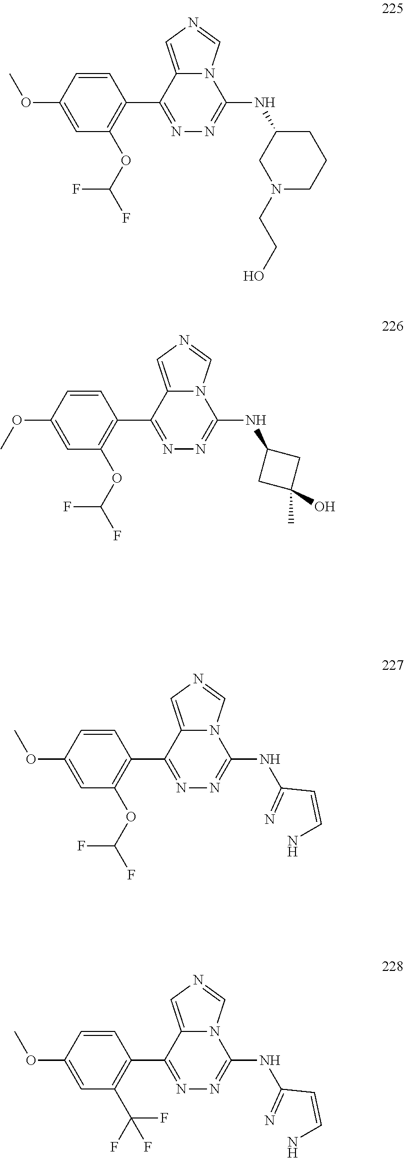 [SMILES: COc1ccc(-c2nnc(N[C@@H]3CCCN(CCO)C3)n3cncc23)c(OC(F)F)c1 ; COc1ccc(-c2nnc(N[C@H]3C[C@@](C)(O)C3)n3cncc23)c(OC(F)F)c1 ; COc1ccc(-c2nnc(NC(=N)/C=C\N)n3cncc23)c(OC(F)F)c1 ; COc1ccc(-c2nnc(NC(=N)/C=C\N)n3cncc23)c(C(F)(F)F)c1]

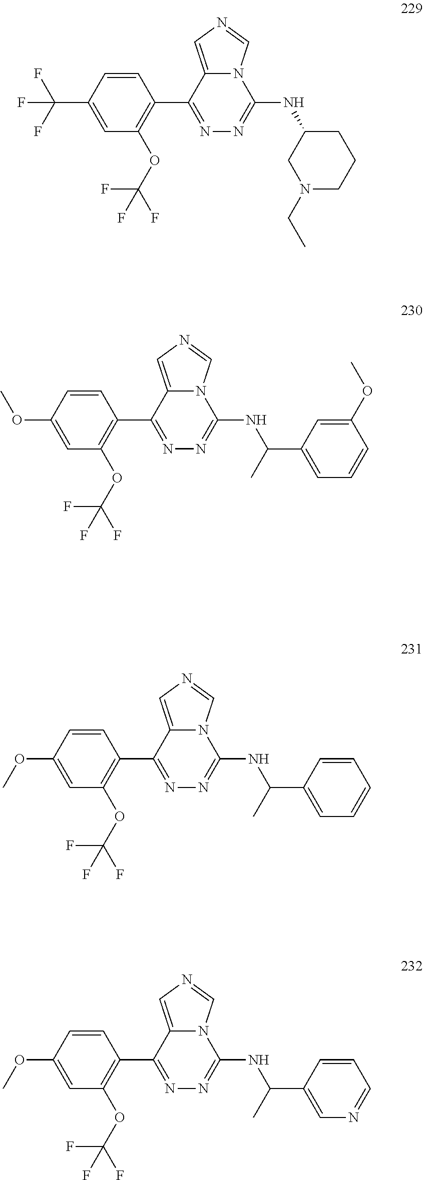 [SMILES: CCN1CCC[C@@H](Nc2nnc(-c3ccc(C(F)(F)F)cc3OC(F)(F)F)c3cncn23)C1 ; COc1cccc(C(C)Nc2nnc(-c3ccc(OC)cc3OC(F)(F)F)c3cncn23)c1 ; COc1ccc(-c2nnc(NC(C)c3ccccc3)n3cncc23)c(OC(F)(F)F)c1 ; COc1ccc(-c2nnc(NC(C)c3cccnc3)n3cncc23)c(OC(F)(F)F)c1]

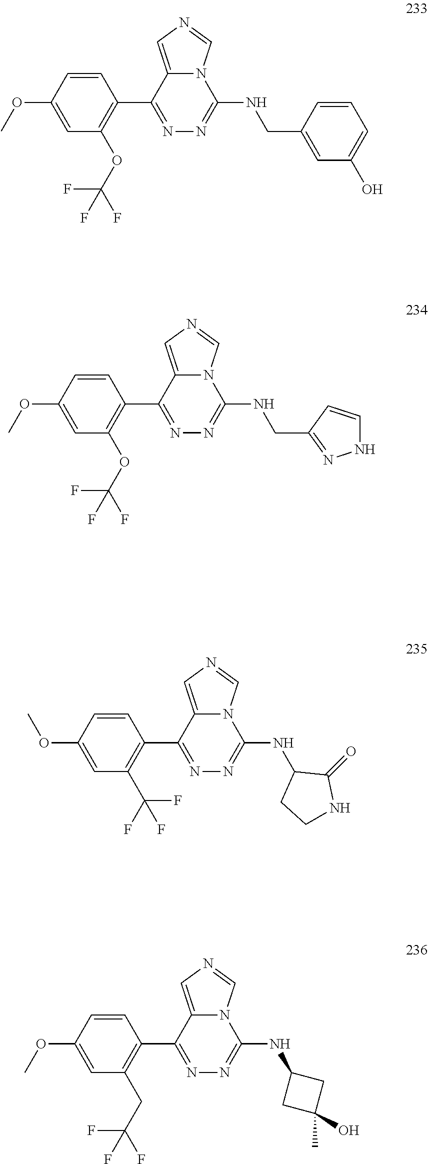 [SMILES: COc1ccc(-c2nnc(NCc3cccc(O)c3)n3cncc23)c(OC(F)(F)F)c1 ; COc1ccc(-c2nnc(NCc3cc[nH]n3)n3cncc23)c(OC(F)(F)F)c1 ; COc1ccc(-c2nnc(NC3CCNC3=O)n3cncc23)c(C(F)(F)F)c1 ; COc1ccc(-c2nnc(N[C@H]3C[C@@](C)(O)C3)n3cncc23)c(CC(F)(F)F)c1]

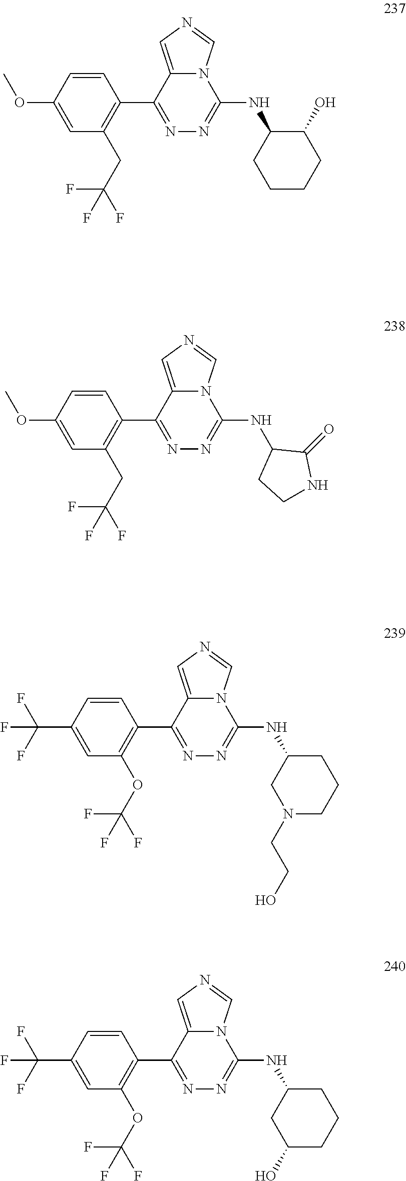 [SMILES: COc1ccc(-c2nnc(N[C@@H]3CCCC[C@H]3O)n3cncc23)c(CC(F)(F)F)c1 ; COc1ccc(-c2nnc(NC3CCNC3=O)n3cncc23)c(CC(F)(F)F)c1 ; OCCN1CCC[C@@H](Nc2nnc(-c3ccc(C(F)(F)F)cc3OC(F)(F)F)c3cncn23)C1 ; O[C@H]1CCC[C@@H](Nc2nnc(-c3ccc(C(F)(F)F)cc3OC(F)(F)F)c3cncn23)C1]

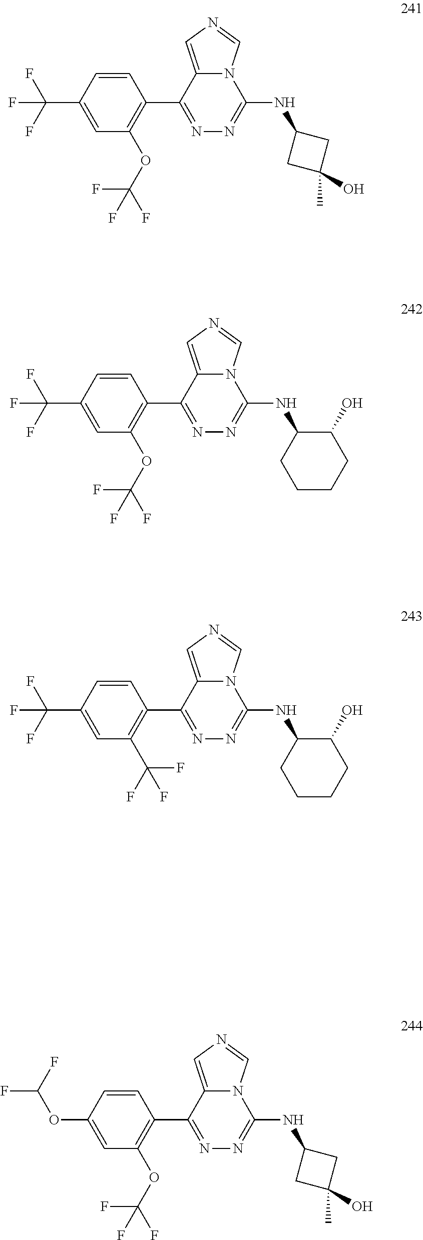 [SMILES: C[C@]1(O)C[C@@H](Nc2nnc(-c3ccc(C(F)(F)F)cc3OC(F)(F)F)c3cncn23)C1 ; O[C@@H]1CCCC[C@H]1Nc1nnc(-c2ccc(C(F)(F)F)cc2OC(F)(F)F)c2cncn12 ; O[C@@H]1CCCC[C@H]1Nc1nnc(-c2ccc(C(F)(F)F)cc2C(F)(F)F)c2cncn12 ; C[C@]1(O)C[C@@H](Nc2nnc(-c3ccc(OC(F)F)cc3OC(F)(F)F)c3cncn23)C1]

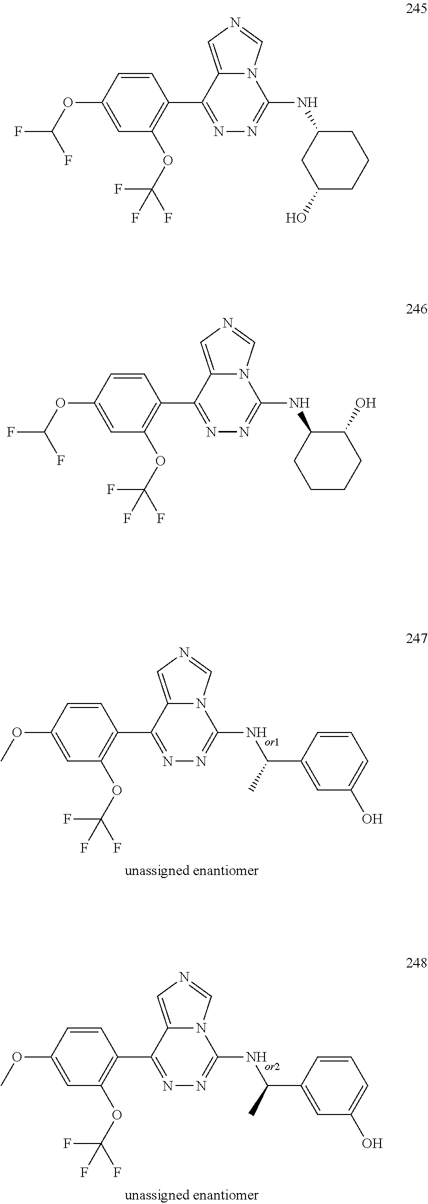 [SMILES: O[C@H]1CCC[C@@H](Nc2nnc(-c3ccc(OC(F)F)cc3OC(F)(F)F)c3cncn23)C1 ; O[C@@H]1CCCC[C@H]1Nc1nnc(-c2ccc(OC(F)F)cc2OC(F)(F)F)c2cncn12 ; COc1ccc(-c2nnc(N[C@@H](C)c3cccc(O)c3)n3cncc23)c(OC(F)(F)F)c1 ; COc1ccc(-c2nnc(N[C@H](C)c3cccc(O)c3)n3cncc23)c(OC(F)(F)F)c1]

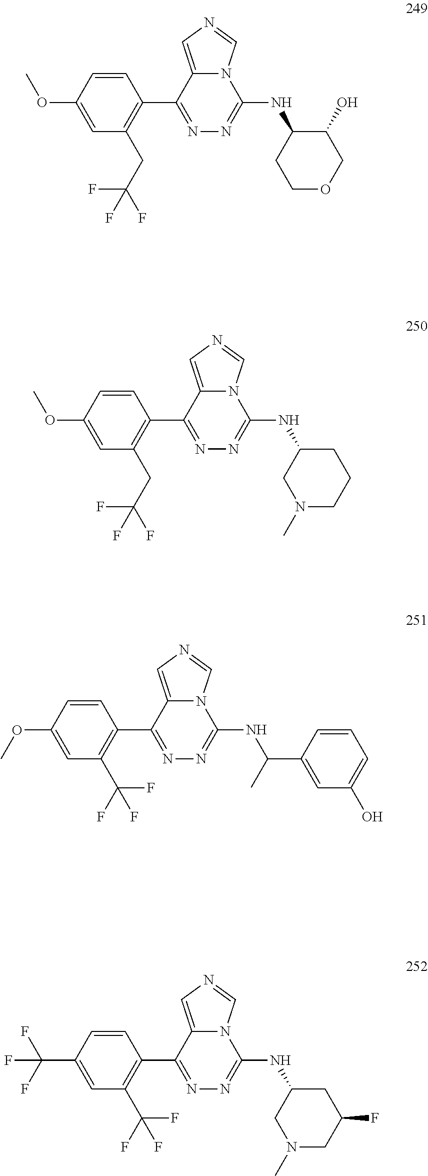 [SMILES: COc1ccc(-c2nnc(N[C@@H]3CCOC[C@H]3O)n3cncc23)c(CC(F)(F)F)c1 ; COc1ccc(-c2nnc(N[C@@H]3CCCN(C)C3)n3cncc23)c(CC(F)(F)F)c1 ; COc1ccc(-c2nnc(NC(C)c3cccc(O)c3)n3cncc23)c(C(F)(F)F)c1 ; CN1C[C@H](F)C[C@@H](Nc2nnc(-c3ccc(C(F)(F)F)cc3C(F)(F)F)c3cncn23)C1]

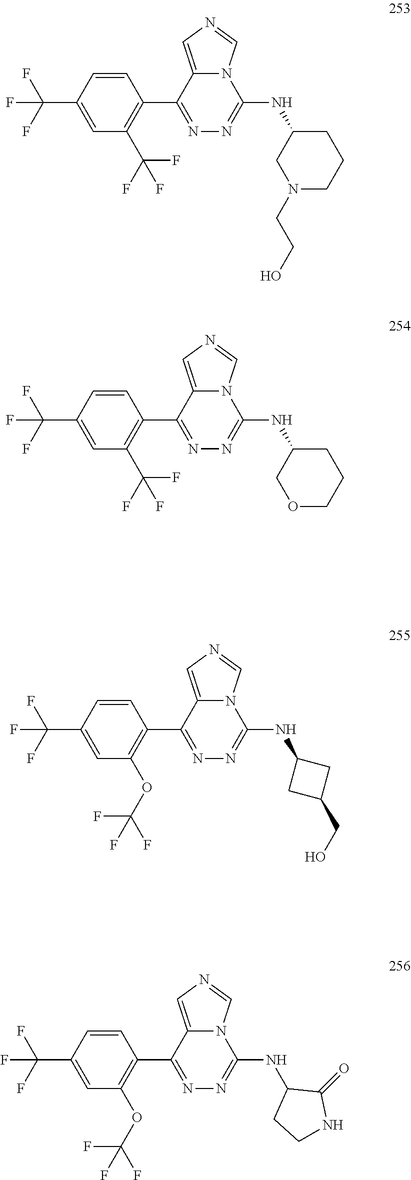 [SMILES: OCCN1CCC[C@@H](Nc2nnc(-c3ccc(C(F)(F)F)cc3C(F)(F)F)c3cncn23)C1 ; FC(F)(F)c1ccc(-c2nnc(N[C@@H]3CCCOC3)n3cncc23)c(C(F)(F)F)c1 ; OC[C@H]1C[C@@H](Nc2nnc(-c3ccc(C(F)(F)F)cc3OC(F)(F)F)c3cncn23)C1 ; O=C1NCCC1Nc1nnc(-c2ccc(C(F)(F)F)cc2OC(F)(F)F)c2cncn12]

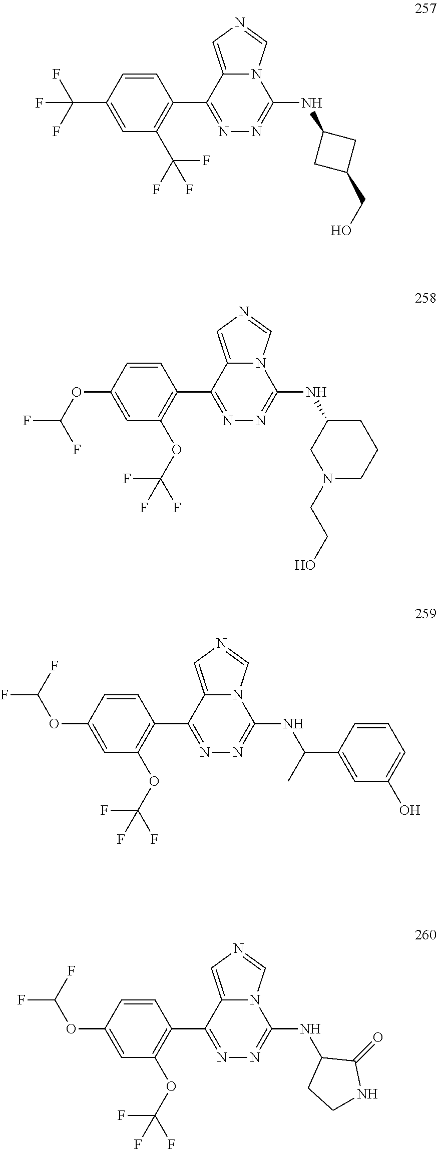 [SMILES: OC[C@H]1C[C@@H](Nc2nnc(-c3ccc(C(F)(F)F)cc3C(F)(F)F)c3cncn23)C1 ; OCCN1CCC[C@@H](Nc2nnc(-c3ccc(OC(F)F)cc3OC(F)(F)F)c3cncn23)C1 ; CC(Nc1nnc(-c2ccc(OC(F)F)cc2OC(F)(F)F)c2cncn12)c1cccc(O)c1 ; O=C1NCCC1Nc1nnc(-c2ccc(OC(F)F)cc2OC(F)(F)F)c2cncn12]

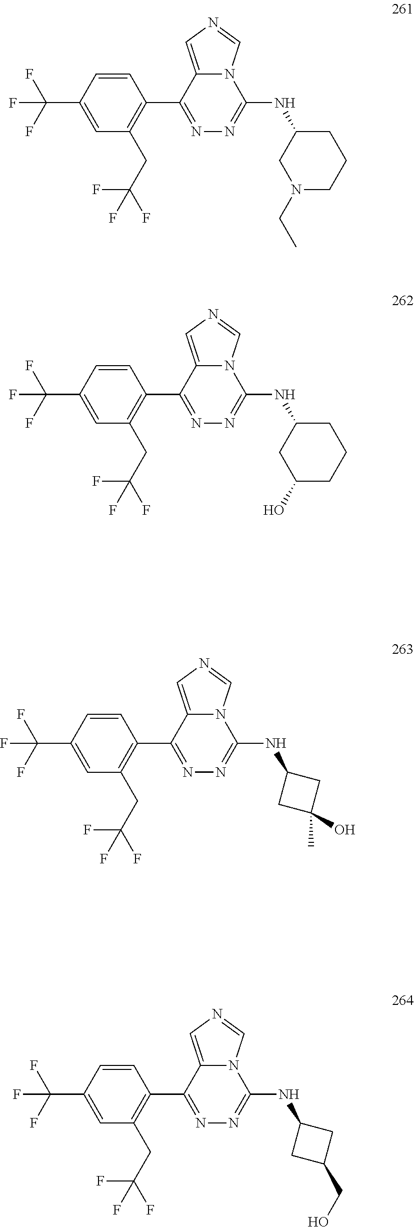 [SMILES: CCN1CCC[C@@H](Nc2nnc(-c3ccc(C(F)(F)F)cc3CC(F)(F)F)c3cncn23)C1 ; O[C@H]1CCC[C@@H](Nc2nnc(-c3ccc(C(F)(F)F)cc3CC(F)(F)F)c3cncn23)C1 ; C[C@]1(O)C[C@@H](Nc2nnc(-c3ccc(C(F)(F)F)cc3CC(F)(F)F)c3cncn23)C1 ; OC[C@H]1C[C@@H](Nc2nnc(-c3ccc(C(F)(F)F)cc3CC(F)(F)F)c3cncn23)C1]

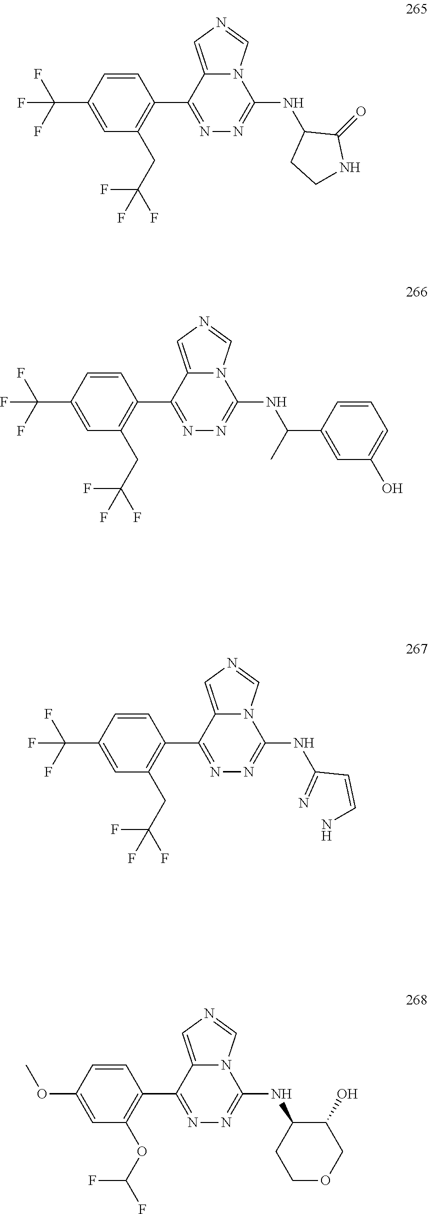 [SMILES: O=C1NCCC1Nc1nnc(-c2ccc(C(F)(F)F)cc2CC(F)(F)F)c2cncn12 ; CC(Nc1nnc(-c2ccc(C(F)(F)F)cc2CC(F)(F)F)c2cncn12)c1cccc(O)c1 ; N=C(/C=C\N)Nc1nnc(-c2ccc(C(F)(F)F)cc2CC(F)(F)F)c2cncn12 ; COc1ccc(-c2nnc(N[C@@H]3CCOC[C@H]3O)n3cncc23)c(OC(F)F)c1]

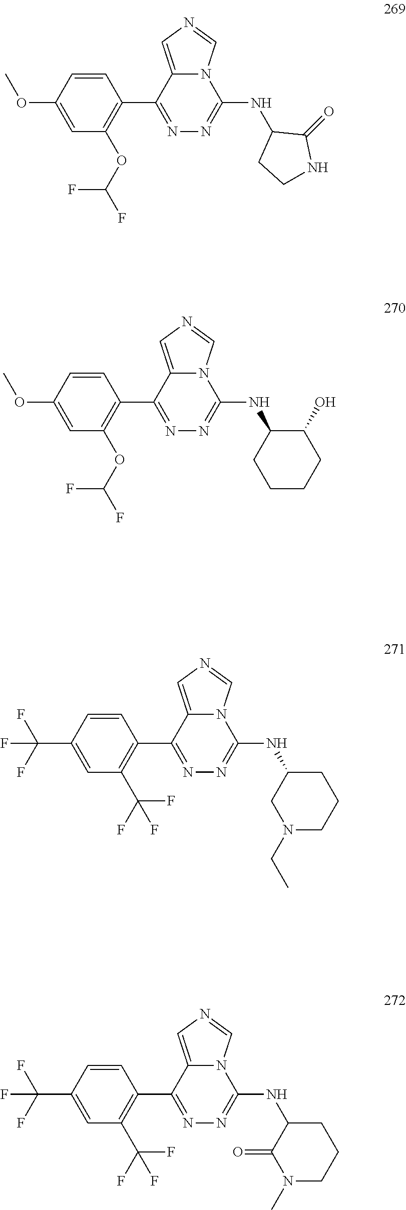 [SMILES: COc1ccc(-c2nnc(NC3CCNC3=O)n3cncc23)c(OC(F)F)c1 ; COc1ccc(-c2nnc(N[C@@H]3CCCC[C@H]3O)n3cncc23)c(OC(F)F)c1 ; CCN1CCC[C@@H](Nc2nnc(-c3ccc(C(F)(F)F)cc3C(F)(F)F)c3cncn23)C1 ; CN1CCCC(Nc2nnc(-c3ccc(C(F)(F)F)cc3C(F)(F)F)c3cncn23)C1=O]

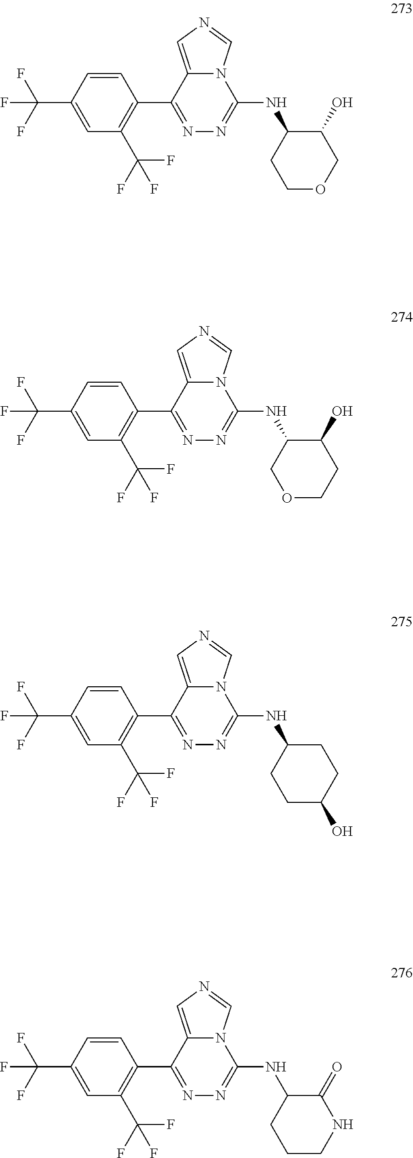 [SMILES: O[C@@H]1COCC[C@H]1Nc1nnc(-c2ccc(C(F)(F)F)cc2C(F)(F)F)c2cncn12 ; O[C@H]1CCOC[C@@H]1Nc1nnc(-c2ccc(C(F)(F)F)cc2C(F)(F)F)c2cncn12 ; O[C@H]1CC[C@@H](Nc2nnc(-c3ccc(C(F)(F)F)cc3C(F)(F)F)c3cncn23)CC1 ; O=C1NCCCC1Nc1nnc(-c2ccc(C(F)(F)F)cc2C(F)(F)F)c2cncn12]

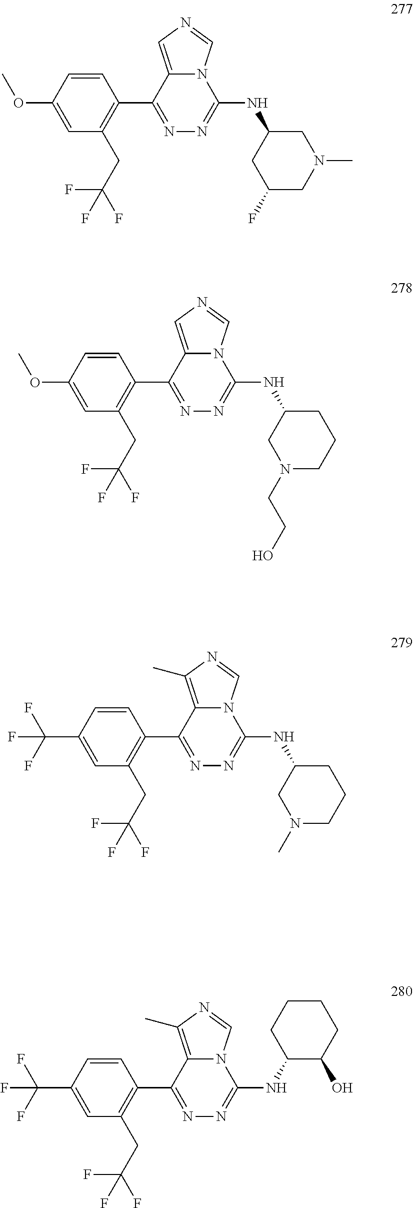

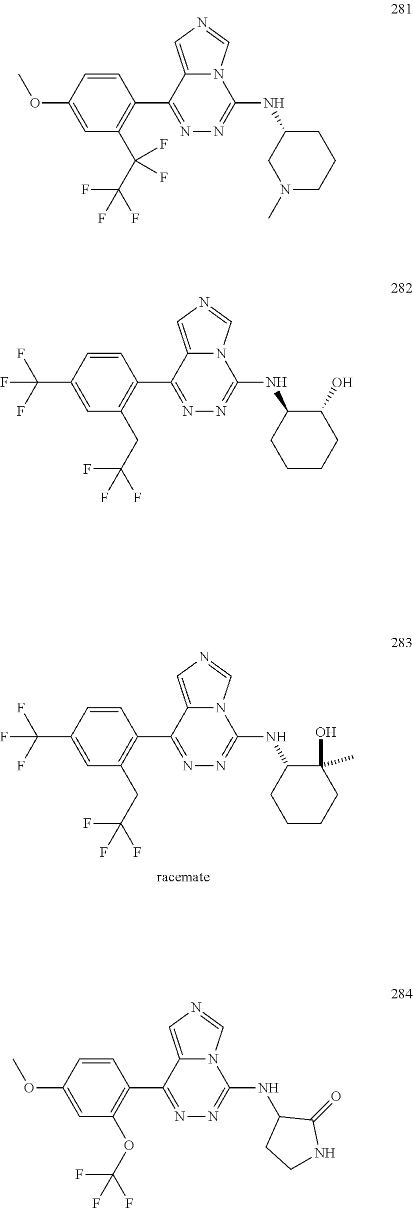

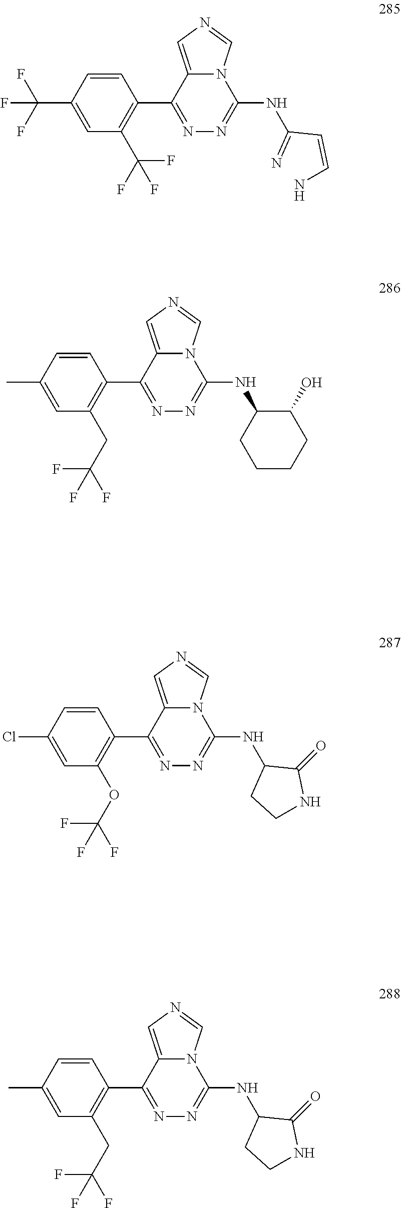 [SMILES: N=C(/C=C\N)Nc1nnc(-c2ccc(C(F)(F)F)cc2C(F)(F)F)c2cncn12 ; Cc1ccc(-c2nnc(N[C@@H]3CCCC[C@H]3O)n3cncc23)c(CC(F)(F)F)c1 ; O=C1NCCC1Nc1nnc(-c2ccc(Cl)cc2OC(F)(F)F)c2cncn12 ; Cc1ccc(-c2nnc(NC3CCNC3=O)n3cncc23)c(CC(F)(F)F)c1]

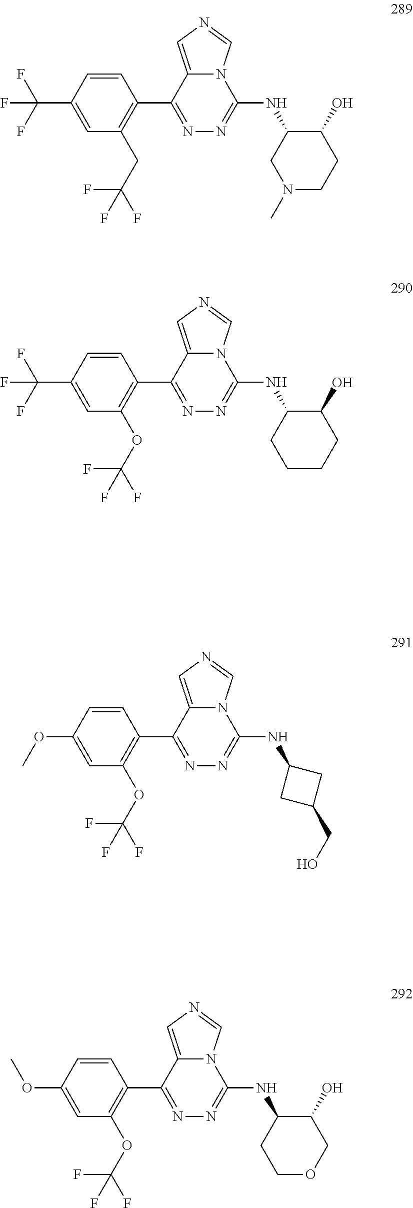 [SMILES: CN1CC[C@@H](O)[C@@H](Nc2nnc(-c3ccc(C(F)(F)F)cc3CC(F)(F)F)c3cncn23)C1 ; O[C@H]1CCCC[C@@H]1Nc1nnc(-c2ccc(C(F)(F)F)cc2OC(F)(F)F)c2cncn12 ; COc1ccc(-c2nnc(N[C@H]3C[C@@H](CO)C3)n3cncc23)c(OC(F)(F)F)c1 ; COc1ccc(-c2nnc(N[C@@H]3CCOC[C@H]3O)n3cncc23)c(OC(F)(F)F)c1]

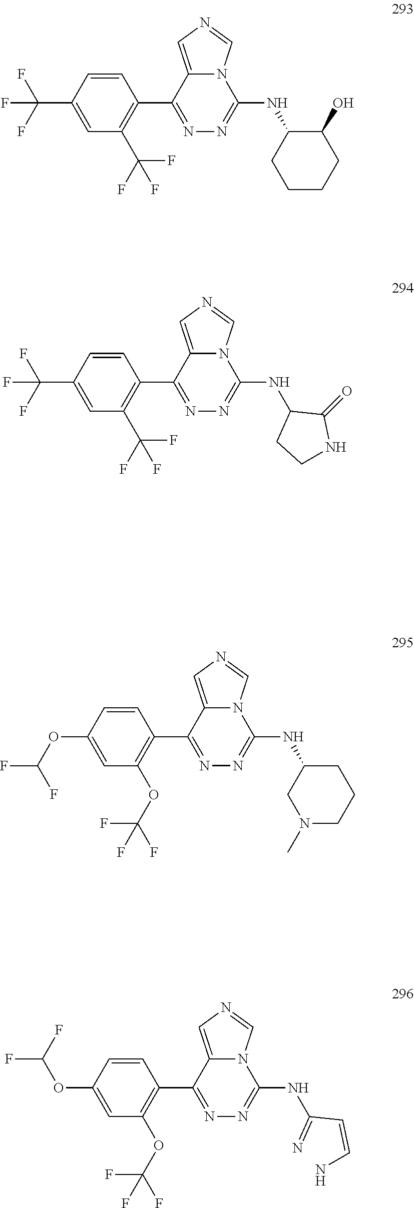 [SMILES: O[C@H]1CCCC[C@@H]1Nc1nnc(-c2ccc(C(F)(F)F)cc2C(F)(F)F)c2cncn12 ; O=C1NCCC1Nc1nnc(-c2ccc(C(F)(F)F)cc2C(F)(F)F)c2cncn12 ; CN1CCC[C@@H](Nc2nnc(-c3ccc(OC(F)F)cc3OC(F)(F)F)c3cncn23)C1 ; N=C(/C=C\N)Nc1nnc(-c2ccc(OC(F)F)cc2OC(F)(F)F)c2cncn12]

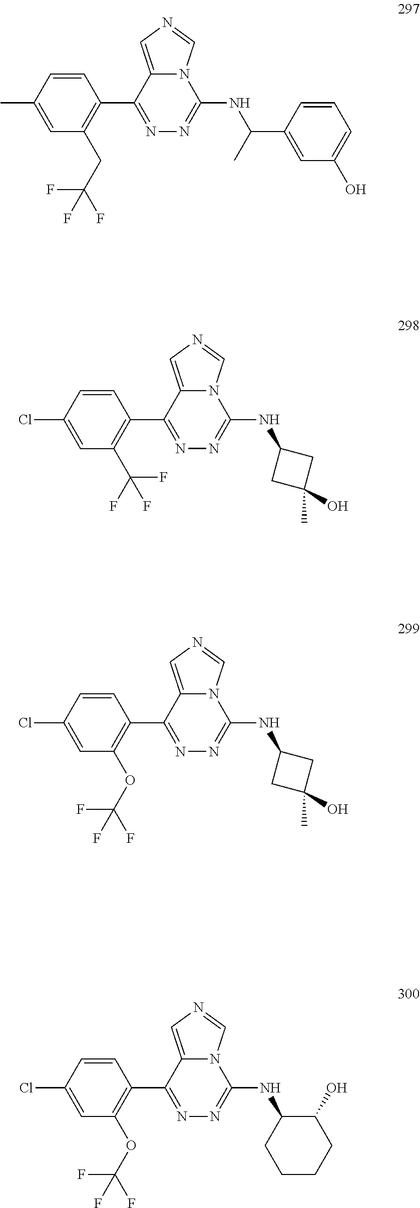 [SMILES: Cc1ccc(-c2nnc(NC(C)c3cccc(O)c3)n3cncc23)c(CC(F)(F)F)c1 ; C[C@]1(O)C[C@@H](Nc2nnc(-c3ccc(Cl)cc3C(F)(F)F)c3cncn23)C1 ; C[C@]1(O)C[C@@H](Nc2nnc(-c3ccc(Cl)cc3OC(F)(F)F)c3cncn23)C1 ; O[C@@H]1CCCC[C@H]1Nc1nnc(-c2ccc(Cl)cc2OC(F)(F)F)c2cncn12]

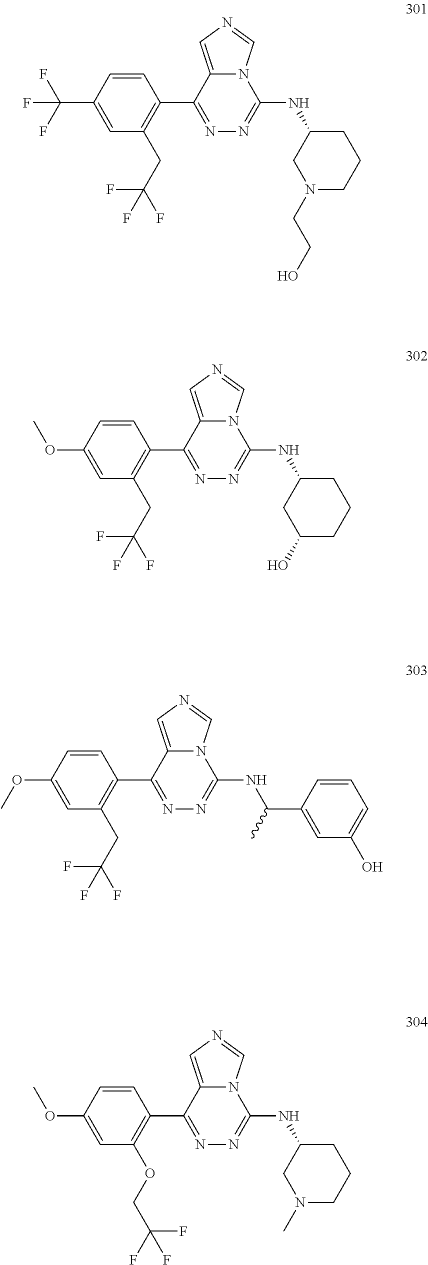 [SMILES: OCCN1CCC[C@@H](Nc2nnc(-c3ccc(C(F)(F)F)cc3CC(F)(F)F)c3cncn23)C1 ; COc1ccc(-c2nnc(N[C@@H]3CCC[C@H](O)C3)n3cncc23)c(CC(F)(F)F)c1 ; COc1ccc(-c2nnc(NC(C)c3cccc(O)c3)n3cncc23)c(CC(F)(F)F)c1 ; COc1ccc(-c2nnc(N[C@@H]3CCCN(C)C3)n3cncc23)c(OCC(F)(F)F)c1]

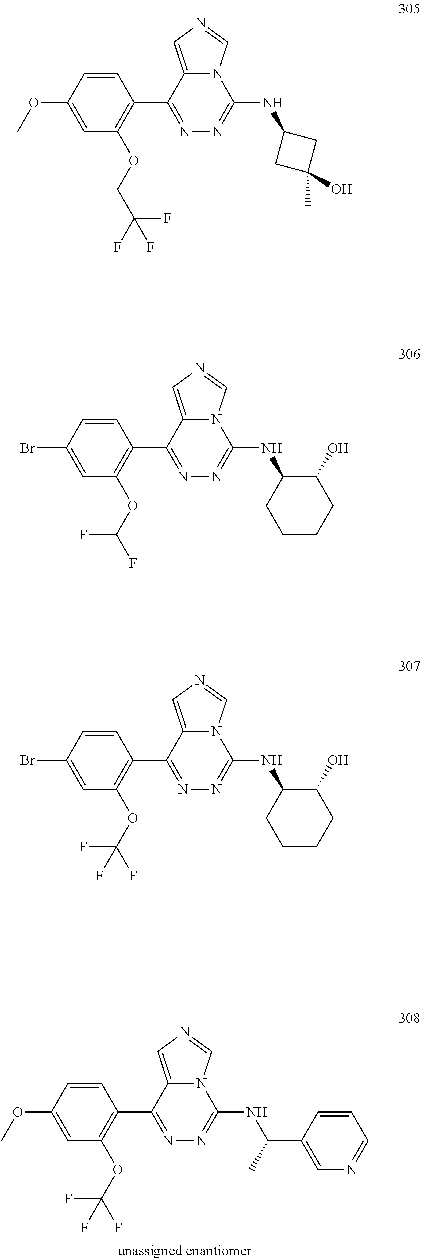 [SMILES: COc1ccc(-c2nnc(N[C@H]3C[C@@](C)(O)C3)n3cncc23)c(OCC(F)(F)F)c1 ; O[C@@H]1CCCC[C@H]1Nc1nnc(-c2ccc(Br)cc2OC(F)F)c2cncn12 ; O[C@@H]1CCCC[C@H]1Nc1nnc(-c2ccc(Br)cc2OC(F)(F)F)c2cncn12 ; COc1ccc(-c2nnc(N[C@@H](C)c3cccnc3)n3cncc23)c(OC(F)(F)F)c1]

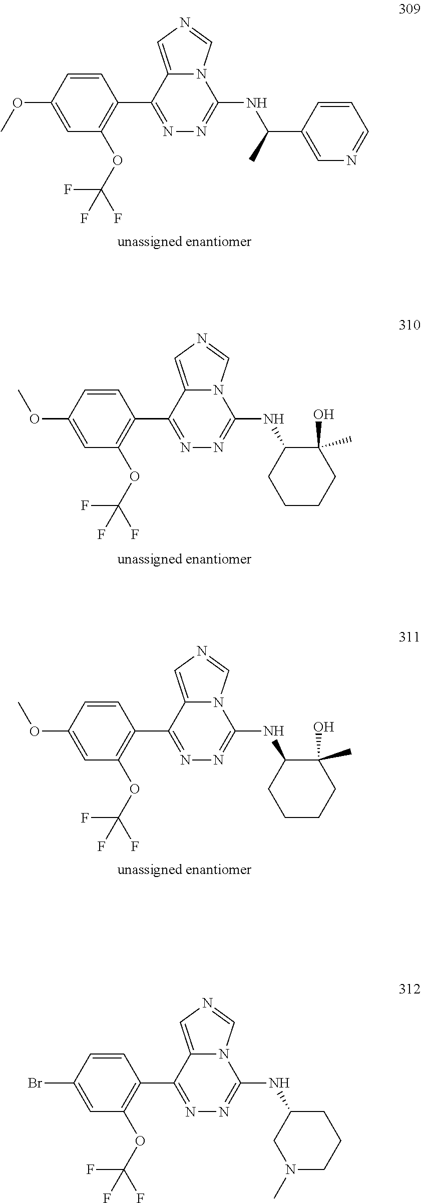

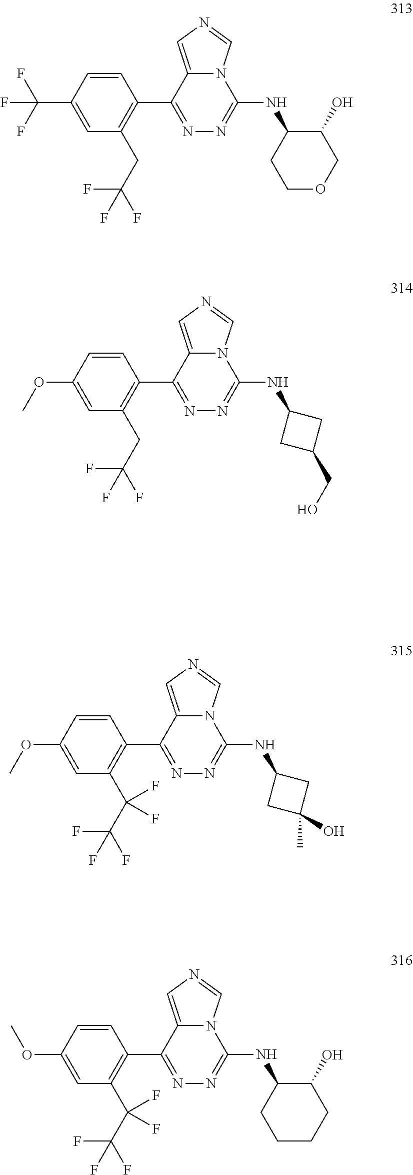 [SMILES: O[C@@H]1COCC[C@H]1Nc1nnc(-c2ccc(C(F)(F)F)cc2CC(F)(F)F)c2cncn12 ; COc1ccc(-c2nnc(N[C@H]3C[C@@H](CO)C3)n3cncc23)c(CC(F)(F)F)c1 ; COc1ccc(-c2nnc(N[C@H]3C[C@@](C)(O)C3)n3cncc23)c(C(F)(F)C(F)(F)F)c1 ; COc1ccc(-c2nnc(N[C@@H]3CCCC[C@H]3O)n3cncc23)c(C(F)(F)C(F)(F)F)c1]

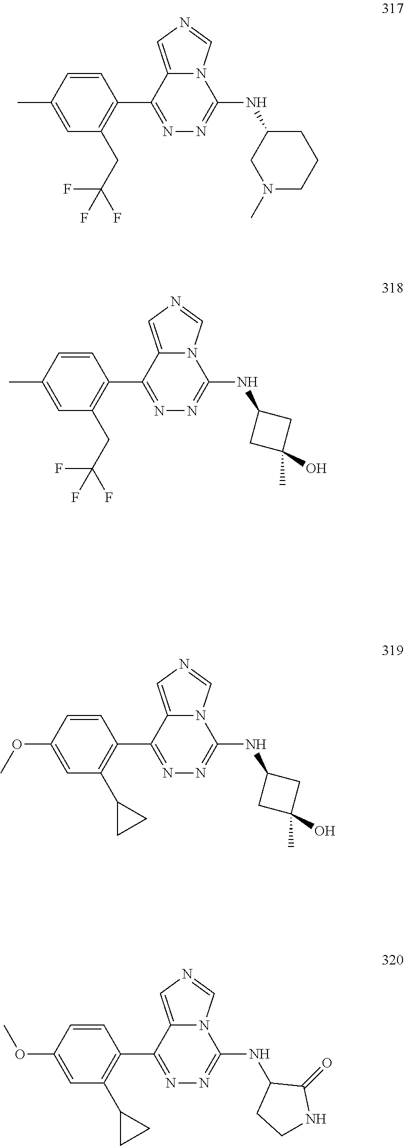 [SMILES: Cc1ccc(-c2nnc(N[C@@H]3CCCN(C)C3)n3cncc23)c(CC(F)(F)F)c1 ; Cc1ccc(-c2nnc(N[C@H]3C[C@@](C)(O)C3)n3cncc23)c(CC(F)(F)F)c1 ; COc1ccc(-c2nnc(N[C@H]3C[C@@](C)(O)C3)n3cncc23)c(C2CC2)c1 ; COc1ccc(-c2nnc(NC3CCNC3=O)n3cncc23)c(C2CC2)c1]

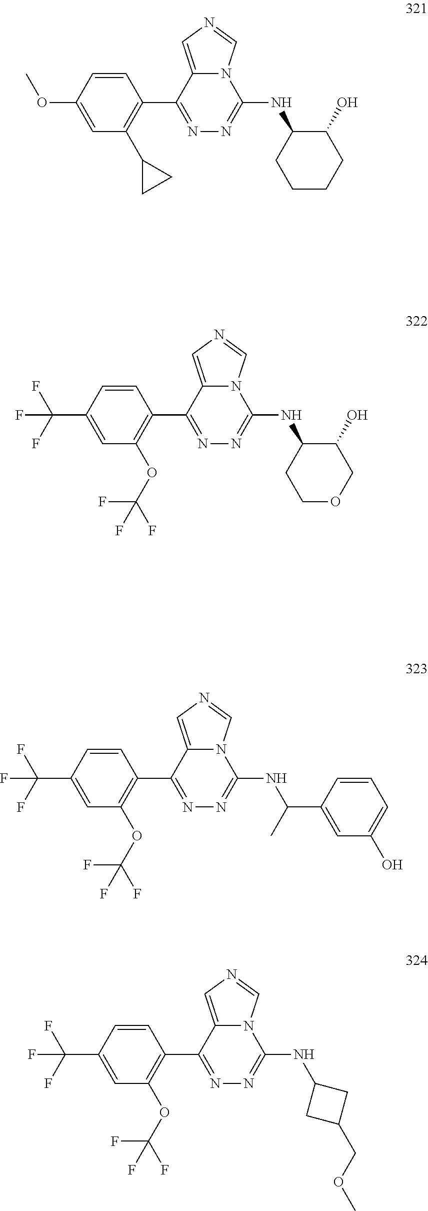 [SMILES: COc1ccc(-c2nnc(N[C@@H]3CCCC[C@H]3O)n3cncc23)c(C2CC2)c1 ; O[C@@H]1COCC[C@H]1Nc1nnc(-c2ccc(C(F)(F)F)cc2OC(F)(F)F)c2cncn12 ; CC(Nc1nnc(-c2ccc(C(F)(F)F)cc2OC(F)(F)F)c2cncn12)c1cccc(O)c1 ; COCC1CC(Nc2nnc(-c3ccc(C(F)(F)F)cc3OC(F)(F)F)c3cncn23)C1]

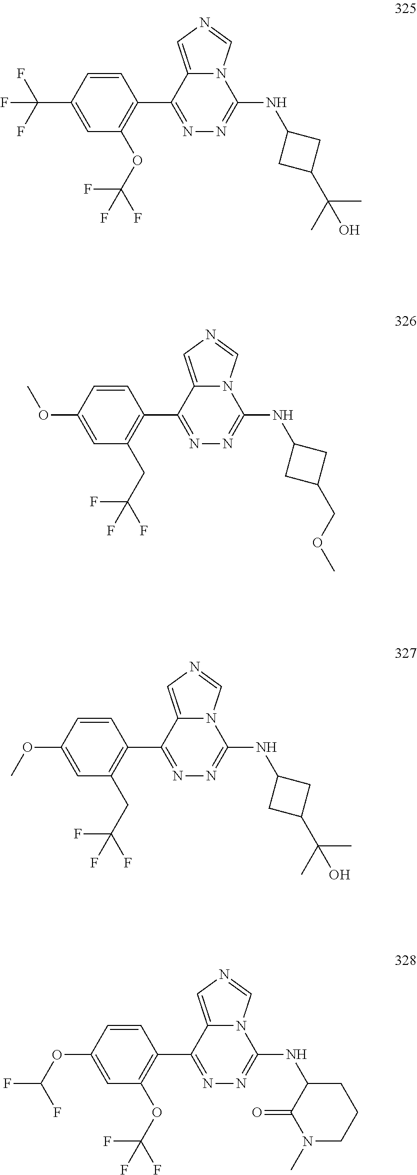

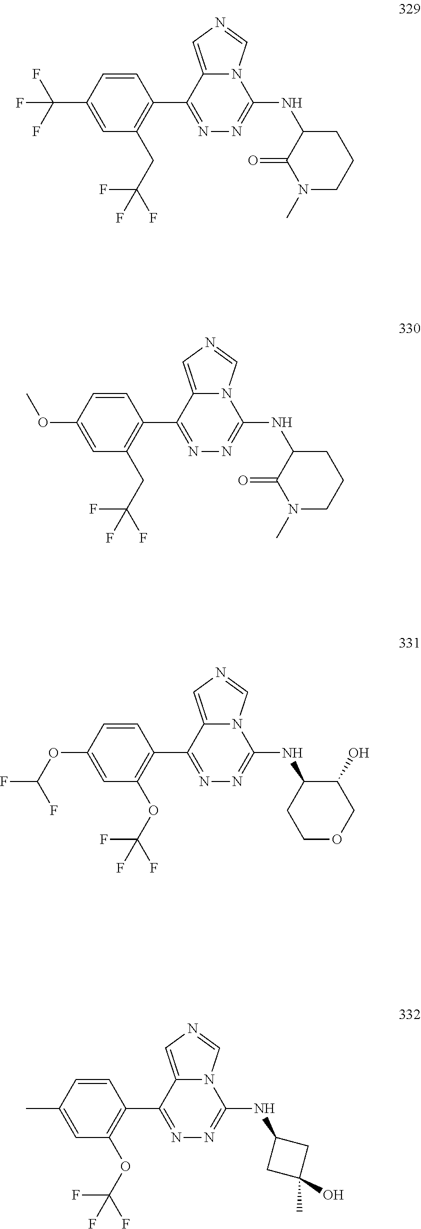 [SMILES: CN1CCCC(Nc2nnc(-c3ccc(C(F)(F)F)cc3CC(F)(F)F)c3cncn23)C1=O ; COc1ccc(-c2nnc(NC3CCCN(C)C3=O)n3cncc23)c(CC(F)(F)F)c1 ; O[C@@H]1COCC[C@H]1Nc1nnc(-c2ccc(OC(F)F)cc2OC(F)(F)F)c2cncn12 ; Cc1ccc(-c2nnc(N[C@H]3C[C@@](C)(O)C3)n3cncc23)c(OC(F)(F)F)c1]

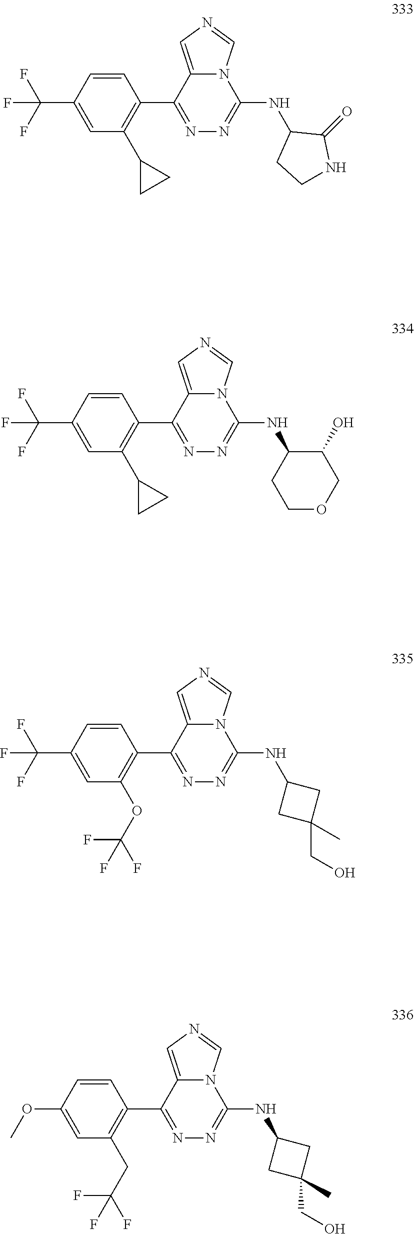

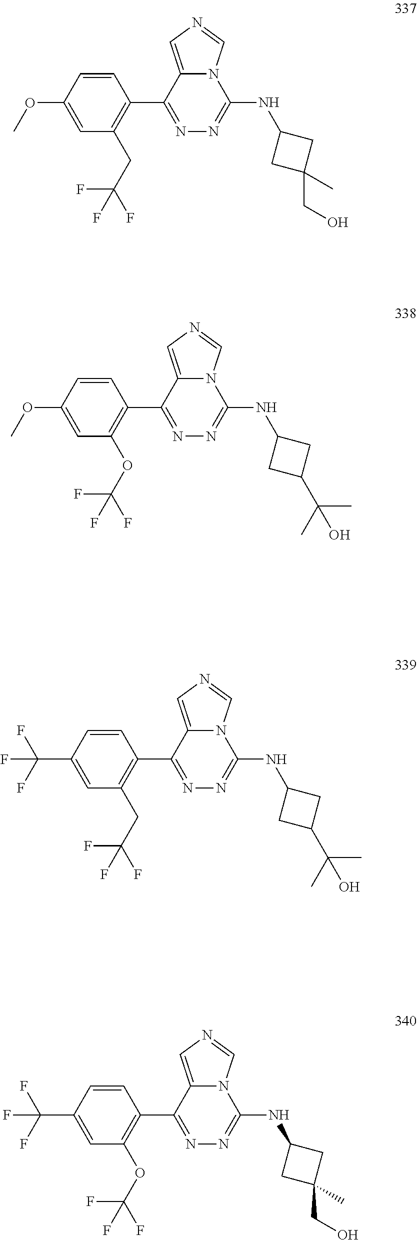

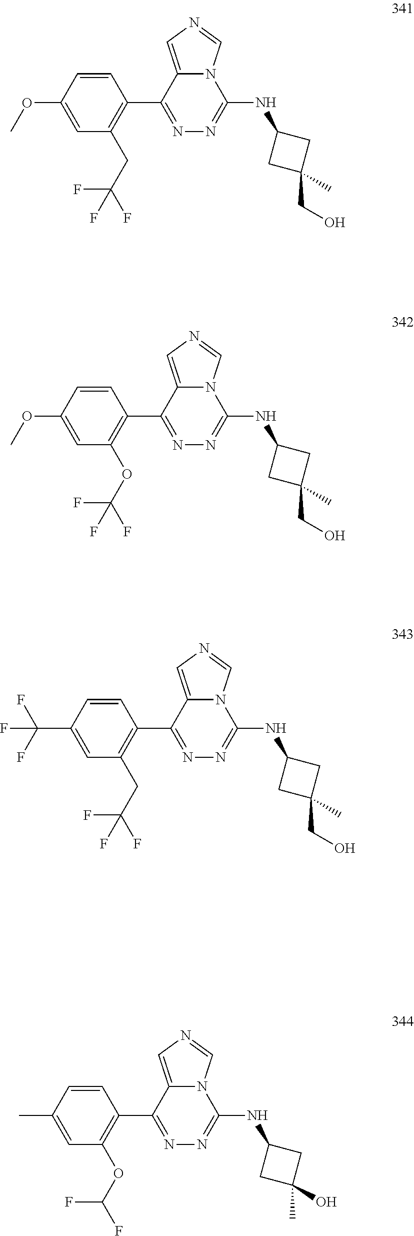

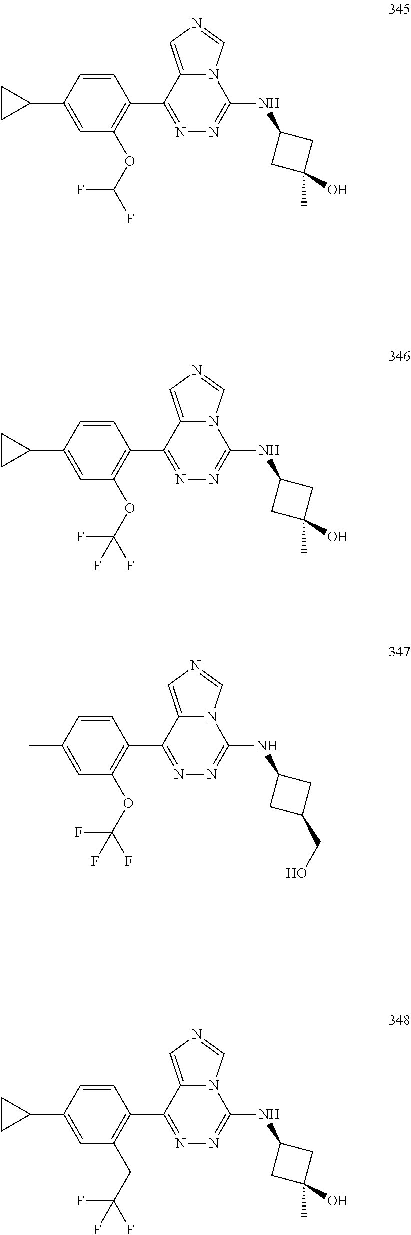 [SMILES: C[C@]1(O)C[C@@H](Nc2nnc(-c3ccc(C4CC4)cc3OC(F)F)c3cncn23)C1 ; C[C@]1(O)C[C@@H](Nc2nnc(-c3ccc(C4CC4)cc3OC(F)(F)F)c3cncn23)C1 ; Cc1ccc(-c2nnc(N[C@H]3C[C@@H](CO)C3)n3cncc23)c(OC(F)(F)F)c1 ; C[C@]1(O)C[C@@H](Nc2nnc(-c3ccc(C4CC4)cc3CC(F)(F)F)c3cncn23)C1]

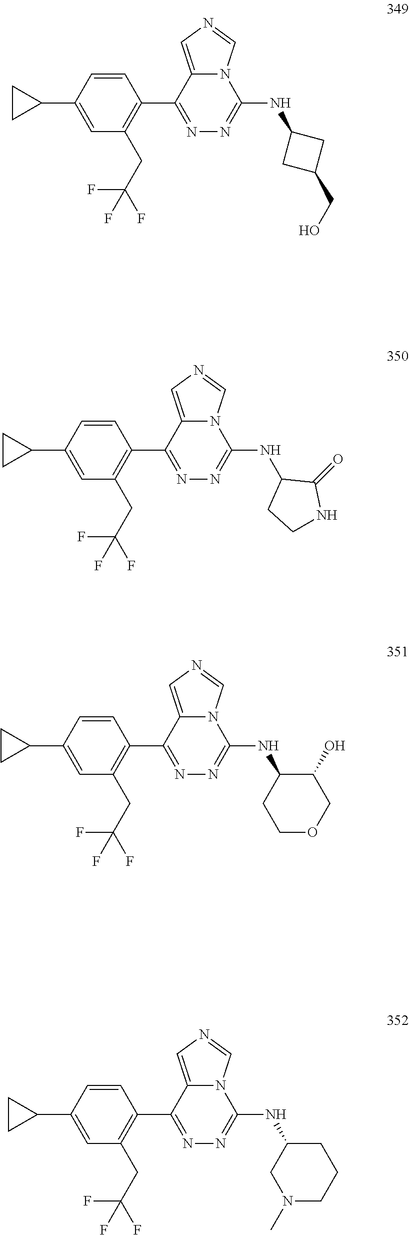 [SMILES: OC[C@H]1C[C@@H](Nc2nnc(-c3ccc(C4CC4)cc3CC(F)(F)F)c3cncn23)C1 ; O=C1NCCC1Nc1nnc(-c2ccc(C3CC3)cc2CC(F)(F)F)c2cncn12 ; O[C@@H]1COCC[C@H]1Nc1nnc(-c2ccc(C3CC3)cc2CC(F)(F)F)c2cncn12 ; CN1CCC[C@@H](Nc2nnc(-c3ccc(C4CC4)cc3CC(F)(F)F)c3cncn23)C1]

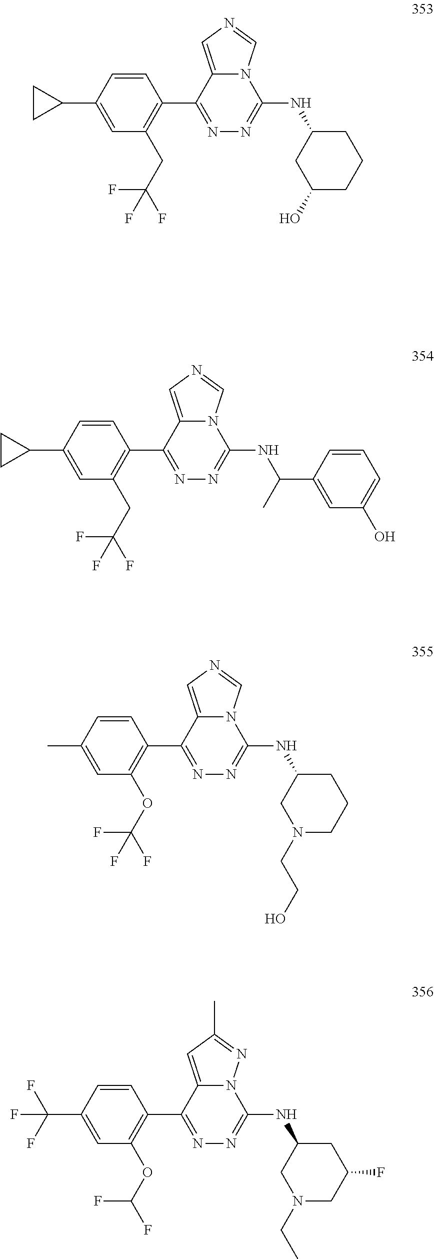 [SMILES: O[C@H]1CCC[C@@H](Nc2nnc(-c3ccc(C4CC4)cc3CC(F)(F)F)c3cncn23)C1 ; CC(Nc1nnc(-c2ccc(C3CC3)cc2CC(F)(F)F)c2cncn12)c1cccc(O)c1 ; Cc1ccc(-c2nnc(N[C@@H]3CCCN(CCO)C3)n3cncc23)c(OC(F)(F)F)c1 ; CCN1C[C@@H](F)C[C@H](Nc2nnc(-c3ccc(C(F)(F)F)cc3OC(F)F)c3cc(C)nn23)C1]

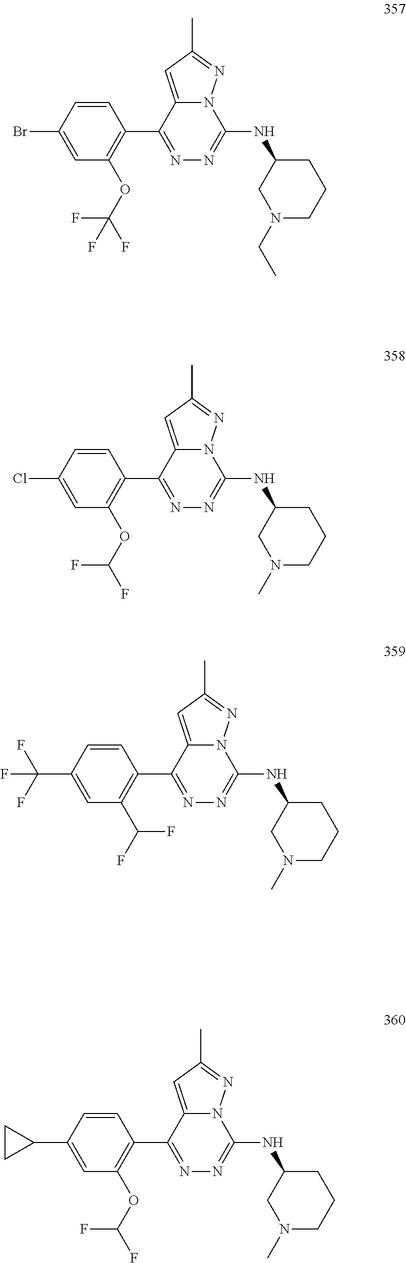 [SMILES: CCN1CCC[C@H](Nc2nnc(-c3ccc(Br)cc3OC(F)(F)F)c3cc(C)nn23)C1 ; Cc1cc2c(-c3ccc(Cl)cc3OC(F)F)nnc(N[C@H]3CCCN(C)C3)n2n1 ; Cc1cc2c(-c3ccc(C(F)(F)F)cc3C(F)F)nnc(N[C@H]3CCCN(C)C3)n2n1 ; Cc1cc2c(-c3ccc(C4CC4)cc3OC(F)F)nnc(N[C@H]3CCCN(C)C3)n2n1]

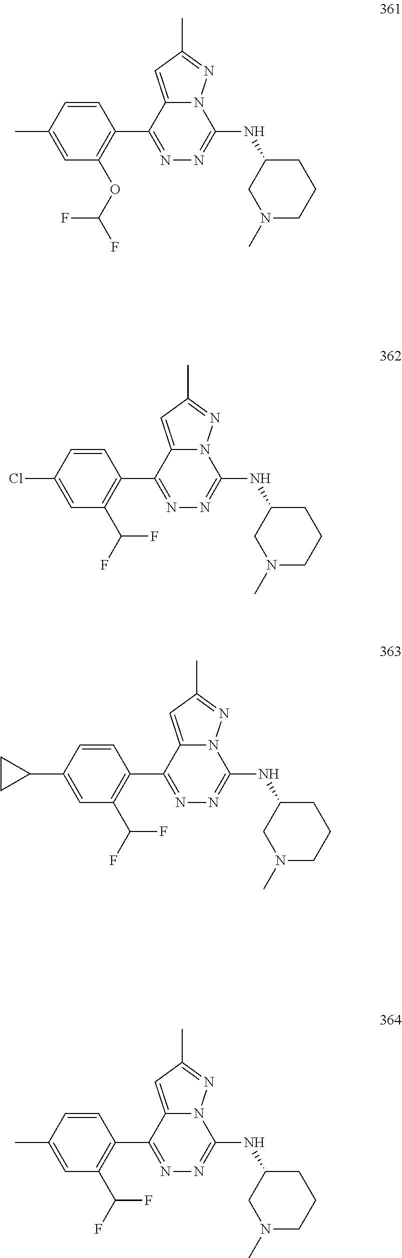 [SMILES: Cc1ccc(-c2nnc(N[C@@H]3CCCN(C)C3)n3nc(C)cc23)c(OC(F)F)c1 ; Cc1cc2c(-c3ccc(Cl)cc3C(F)F)nnc(N[C@@H]3CCCN(C)C3)n2n1 ; Cc1cc2c(-c3ccc(C4CC4)cc3C(F)F)nnc(N[C@@H]3CCCN(C)C3)n2n1 ; Cc1ccc(-c2nnc(N[C@@H]3CCCN(C)C3)n3nc(C)cc23)c(C(F)F)c1]

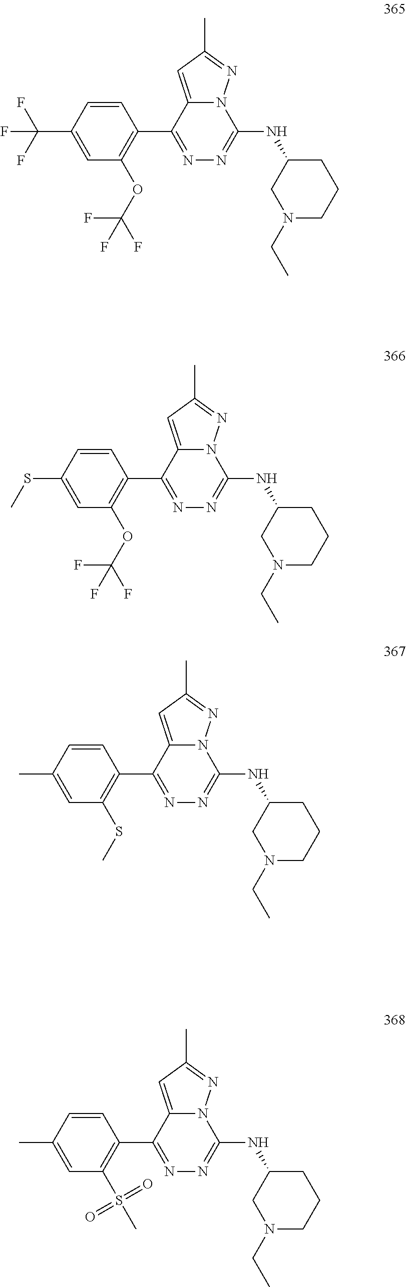 [SMILES: CCN1CCC[C@@H](Nc2nnc(-c3ccc(C(F)(F)F)cc3OC(F)(F)F)c3cc(C)nn23)C1 ; CCN1CCC[C@@H](Nc2nnc(-c3ccc(SC)cc3OC(F)(F)F)c3cc(C)nn23)C1 ; CCN1CCC[C@@H](Nc2nnc(-c3ccc(C)cc3SC)c3cc(C)nn23)C1 ; CCN1CCC[C@@H](Nc2nnc(-c3ccc(C)cc3S(C)(=O)=O)c3cc(C)nn23)C1]

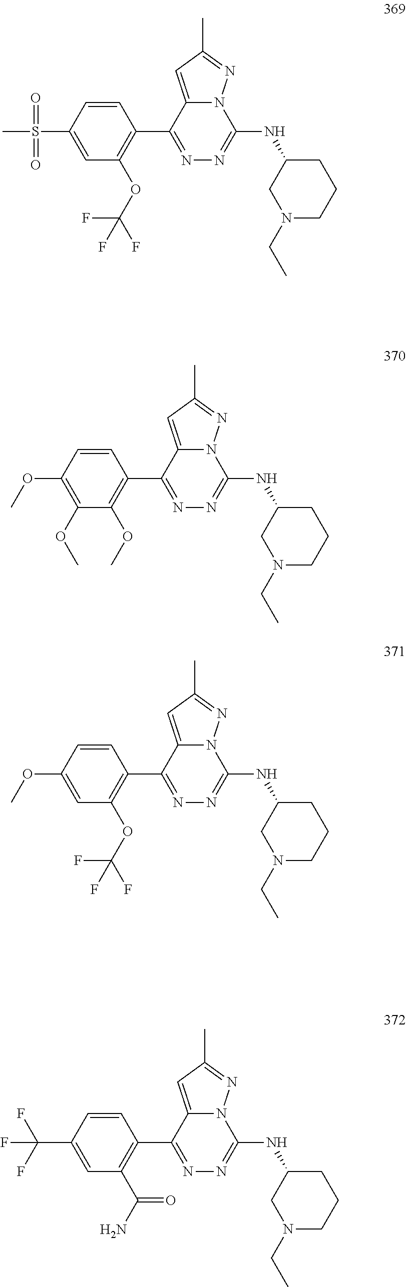 [SMILES: CCN1CCC[C@@H](Nc2nnc(-c3ccc(S(C)(=O)=O)cc3OC(F)(F)F)c3cc(C)nn23)C1 ; CCN1CCC[C@@H](Nc2nnc(-c3ccc(OC)c(OC)c3OC)c3cc(C)nn23)C1 ; CCN1CCC[C@@H](Nc2nnc(-c3ccc(OC)cc3OC(F)(F)F)c3cc(C)nn23)C1 ; CCN1CCC[C@@H](Nc2nnc(-c3ccc(C(F)(F)F)cc3C(N)=O)c3cc(C)nn23)C1]

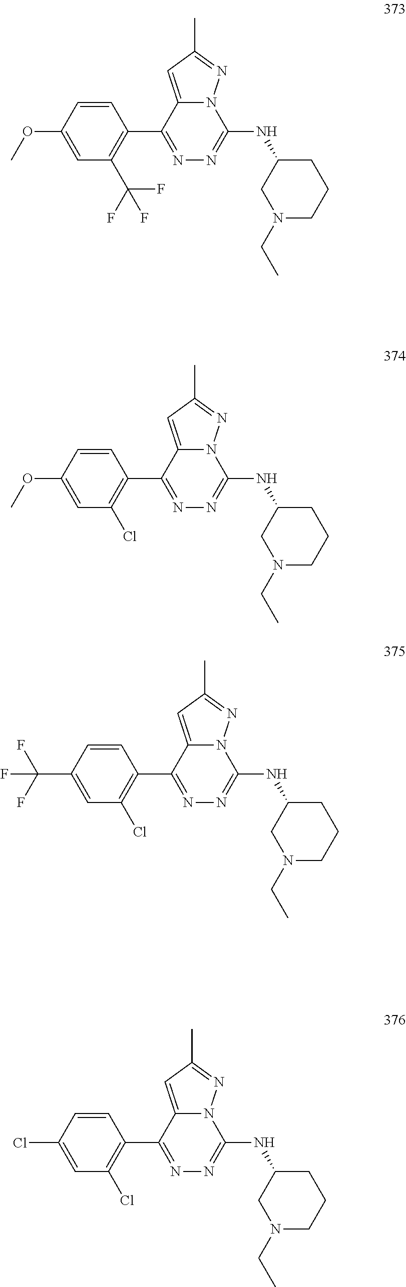 [SMILES: CCN1CCC[C@@H](Nc2nnc(-c3ccc(OC)cc3C(F)(F)F)c3cc(C)nn23)C1 ; CCN1CCC[C@@H](Nc2nnc(-c3ccc(OC)cc3Cl)c3cc(C)nn23)C1 ; CCN1CCC[C@@H](Nc2nnc(-c3ccc(C(F)(F)F)cc3Cl)c3cc(C)nn23)C1 ; CCN1CCC[C@@H](Nc2nnc(-c3ccc(Cl)cc3Cl)c3cc(C)nn23)C1]

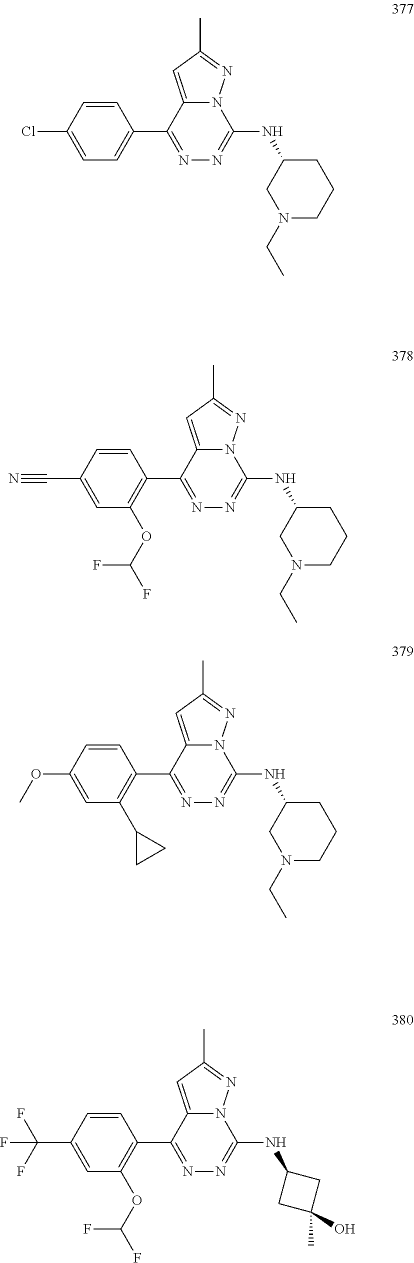 [SMILES: CCN1CCC[C@@H](Nc2nnc(-c3ccc(Cl)cc3)c3cc(C)nn23)C1 ; CCN1CCC[C@@H](Nc2nnc(-c3ccc(C#N)cc3OC(F)F)c3cc(C)nn23)C1 ; CCN1CCC[C@@H](Nc2nnc(-c3ccc(OC)cc3C3CC3)c3cc(C)nn23)C1 ; Cc1cc2c(-c3ccc(C(F)(F)F)cc3OC(F)F)nnc(N[C@H]3C[C@@](C)(O)C3)n2n1]

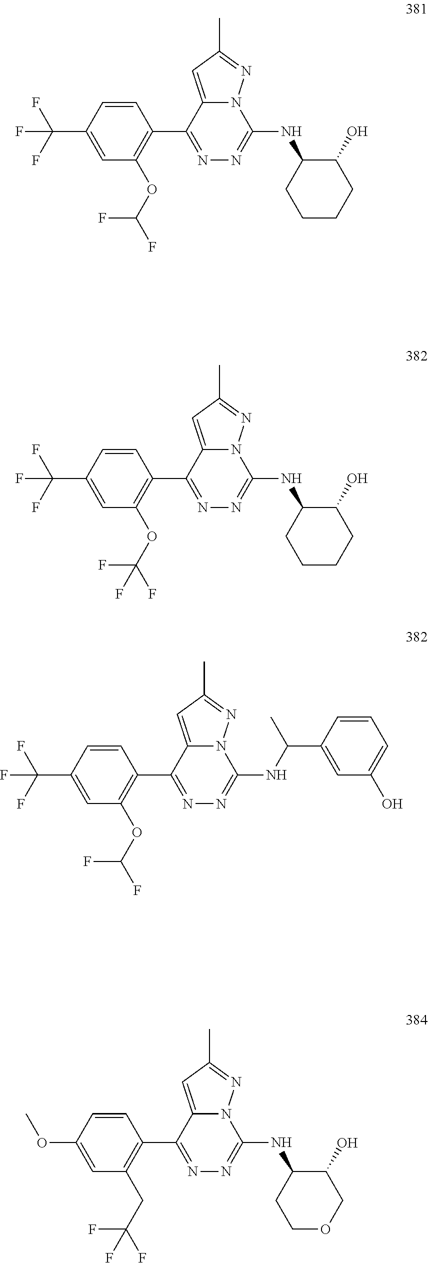 [SMILES: Cc1cc2c(-c3ccc(C(F)(F)F)cc3OC(F)F)nnc(N[C@@H]3CCCC[C@H]3O)n2n1 ; Cc1cc2c(-c3ccc(C(F)(F)F)cc3OC(F)(F)F)nnc(N[C@@H]3CCCC[C@H]3O)n2n1 ; Cc1cc2c(-c3ccc(C(F)(F)F)cc3OC(F)F)nnc(NC(C)c3cccc(O)c3)n2n1 ; COc1ccc(-c2nnc(N[C@@H]3CCOC[C@H]3O)n3nc(C)cc23)c(CC(F)(F)F)c1]

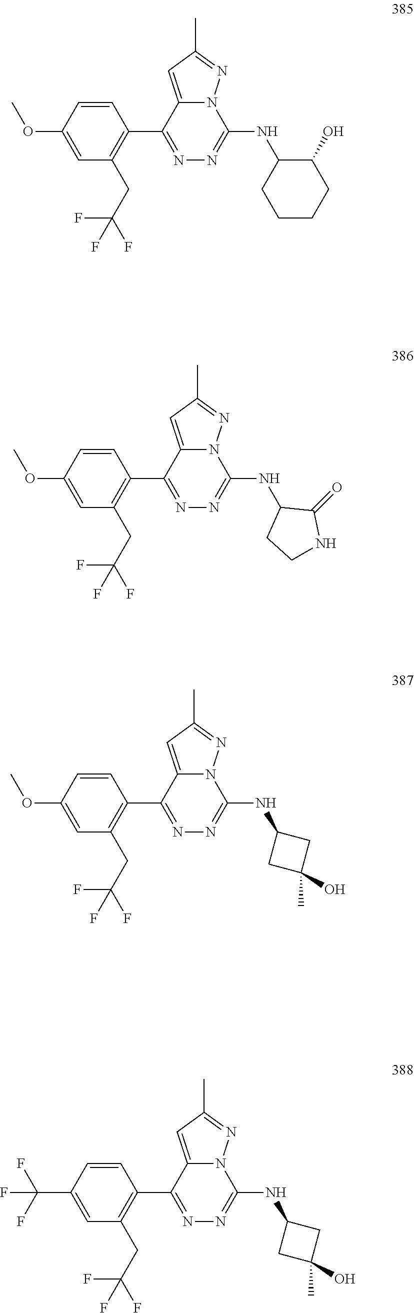 [SMILES: COc1ccc(-c2nnc(NC3CCCC[C@H]3O)n3nc(C)cc23)c(CC(F)(F)F)c1 ; COc1ccc(-c2nnc(NC3CCNC3=O)n3nc(C)cc23)c(CC(F)(F)F)c1 ; COc1ccc(-c2nnc(N[C@H]3C[C@@](C)(O)C3)n3nc(C)cc23)c(CC(F)(F)F)c1 ; Cc1cc2c(-c3ccc(C(F)(F)F)cc3CC(F)(F)F)nnc(N[C@H]3C[C@@](C)(O)C3)n2n1]

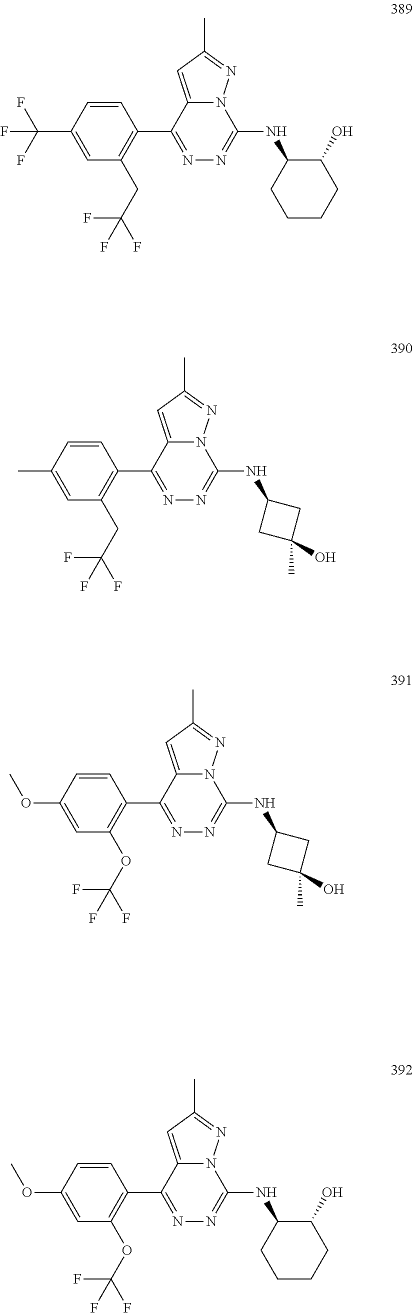 [SMILES: Cc1cc2c(-c3ccc(C(F)(F)F)cc3CC(F)(F)F)nnc(N[C@@H]3CCCC[C@H]3O)n2n1 ; Cc1ccc(-c2nnc(N[C@H]3C[C@@](C)(O)C3)n3nc(C)cc23)c(CC(F)(F)F)c1 ; COc1ccc(-c2nnc(N[C@H]3C[C@@](C)(O)C3)n3nc(C)cc23)c(OC(F)(F)F)c1 ; COc1ccc(-c2nnc(N[C@@H]3CCCC[C@H]3O)n3nc(C)cc23)c(OC(F)(F)F)c1]

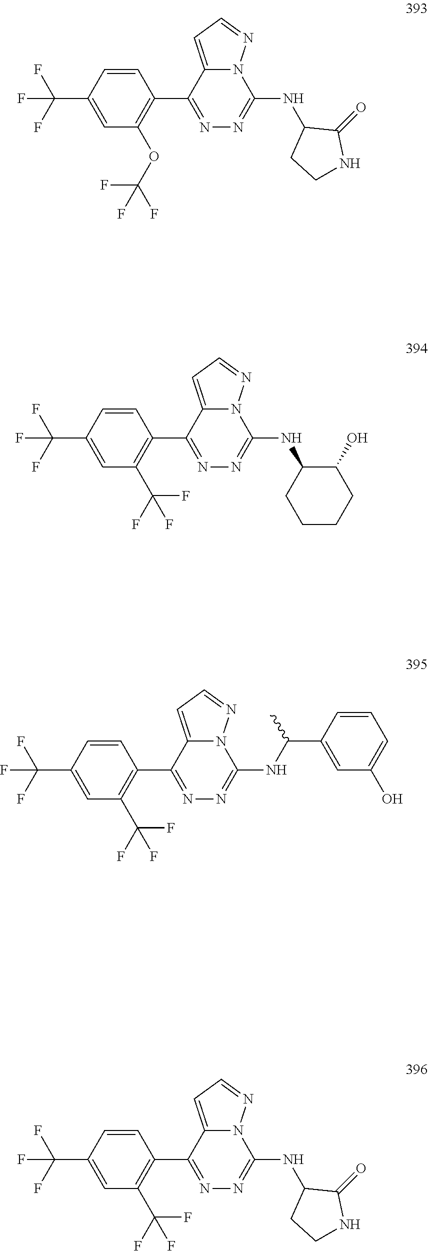 [SMILES: O=C1NCCC1Nc1nnc(-c2ccc(C(F)(F)F)cc2OC(F)(F)F)c2ccnn12 ; O[C@@H]1CCCC[C@H]1Nc1nnc(-c2ccc(C(F)(F)F)cc2C(F)(F)F)c2ccnn12 ; CC(Nc1nnc(-c2ccc(C(F)(F)F)cc2C(F)(F)F)c2ccnn12)c1cccc(O)c1 ; O=C1NCCC1Nc1nnc(-c2ccc(C(F)(F)F)cc2C(F)(F)F)c2ccnn12]

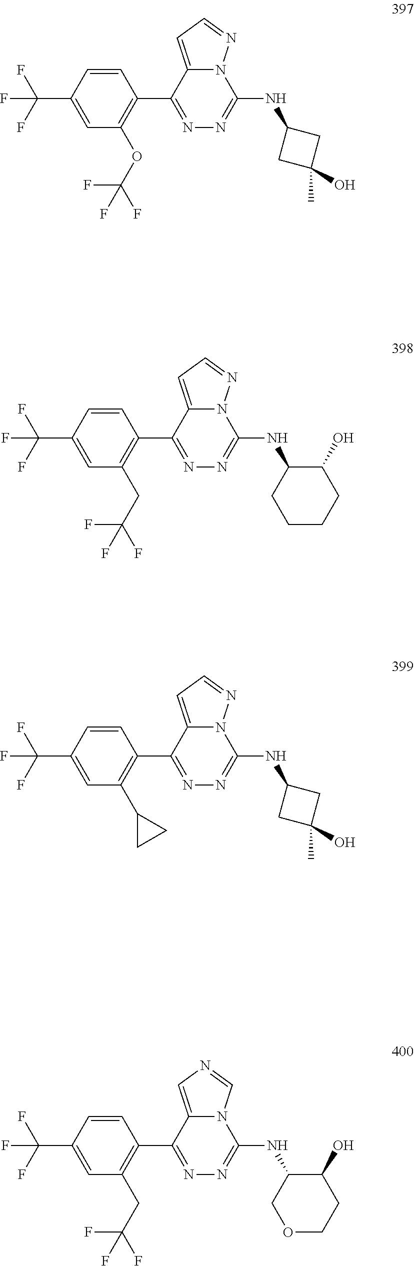 [SMILES: C[C@]1(O)C[C@@H](Nc2nnc(-c3ccc(C(F)(F)F)cc3OC(F)(F)F)c3ccnn23)C1 ; O[C@@H]1CCCC[C@H]1Nc1nnc(-c2ccc(C(F)(F)F)cc2CC(F)(F)F)c2ccnn12 ; C[C@]1(O)C[C@@H](Nc2nnc(-c3ccc(C(F)(F)F)cc3C3CC3)c3ccnn23)C1 ; O[C@H]1CCOC[C@@H]1Nc1nnc(-c2ccc(C(F)(F)F)cc2CC(F)(F)F)c2cncn12]

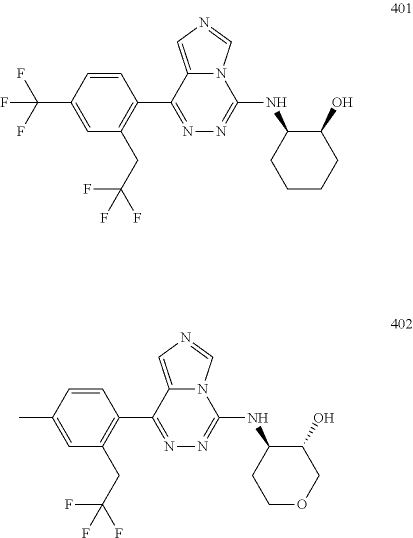 [SMILES: O[C@H]1CCCC[C@H]1Nc1nnc(-c2ccc(C(F)(F)F)cc2CC(F)(F)F)c2cncn12 ; Cc1ccc(-c2nnc(N[C@@H]3CCOC[C@H]3O)n3cncc23)c(CC(F)(F)F)c1]

wherein a form of the compound is selected from the group consisting of a pharmaceutically acceptable salt, hydrate, solvate, racemate, enantiomer, diastereomer, stereoisomer, tautomer, and isotope enriched form thereof.

In embodiment 1, the invention provides compounds, or pharmaceutically acceptable salts thereof, as described above.

In embodiment 2, the invention provides compounds selected from:

| Compound# | Name |
|---|---|
| 1 | (1s,3s)-3-({1-[4-methoxy-2-(2,2,2-trifluoroethyl)phenyl]pyrrolo[1,2-d][1,2,4]triazin- |
| | 4-yl}amino)-1-methylcyclobutan-1-ol |
| 2 | N-[(3R)-1-methylpiperidin-3-yl]-1-[2-(2,2,2-trifluoroethyl)-4- |
| | (trifluoromethyl)phenyl]imidazo[1,5-d][1,2,4]triazin-4-amine |
| 3 | (1s,3s)-1-methyl-3-({2-methyl-4-[2-(trifluoromethoxy)-4- |
| | (trifluoromethyl)phenyl]pyrazolo[1,5-d][1,2,4]triazin-7-yl}amino)cyclobutan-1-ol |
| 4 | (1s,3s)-3-({4-[2,4-bis(trifluoromethyl)phenyl]pyrazolo[1,5-d][1,2,4]triazin-7- |
| | yl}amino)-1-methylcyclobutan-1-ol |
| 5 | 1-[2-(difluoromethoxy)-4-(trifluoromethyl)phenyl]-N-[(3R,5R)-1-ethyl-5- |
| | fluoropiperidin-3-yl]-8-fluoropyrrolo[1,2-d][1,2,4]triazin-4-amine |
| 6 | 1-[2,4-bis(trifluoromethyl)phenyl]-N-[(3R)-1-(oxan-3-yl)piperidin-3-yl]pyrrolo[1,2- |
| | d][1,2,4]triazin-4-amine |
| 7 | 1-{1-[2,4-bis(trifluoromethyl)phenyl]pyrrolo[1,2-d][1,2,4]triazin-4-yl}-N,N- |
| | dimethylpiperidin-3-amine |
| 8 | 1-[4-methoxy-2-(trifluoromethoxy)phenyl]-N-[(3R)-1-methylpiperidin-3- |
| | yl]pyrrolo[1,2-d][1,2,4]triazin-4-amine |
| 9 | (1s,3s)-3-({1-[4-methoxy-2-(trifluoromethoxy)phenyl]pyrrolo[1,2-d][1,2,4]triazin-4- |
| | yl}amino)-1-methylcyclobutan-1-ol |
| 10 | 1-[2,4-bis(trifluoromethyl)phenyl]-N-[(3R)-1-(2-methylpropyl)piperidin-3- |
| | yl]pyrrolo[1,2-d][1,2,4]triazin-4-amine |
| 11 | 1-[4-chloro-2-(difluoromethyl)phenyl]-7-methyl-N-[(3R)-1-methylpiperidin-3- |
| | yl]pyrrolo[1,2-d][1,2,4]triazin-4-amine |
| 12 | 1-[2-(difluoromethoxy)-4-methoxyphenyl]-7-methyl-N-[(3R)-1-methylpiperidin-3- |
| | yl]pyrrolo[1,2-d][1,2,4]triazin-4-amine |
| 13 | 1-[4-chloro-2-(difluoromethoxy)phenyl]-7-methyl-N-[(3R)-1-methylpiperidin-3- |
| | yl]pyrrolo[1,2-d][1,2,4]triazin-4-amine |
| 14 | 1-[4-cyclopropyl-2-(difluoromethyl)phenyl]-7-methyl-N-[(3R)-1-methylpiperidin-3- |
| | yl]pyrrolo[1,2-d][1,2,4]triazin-4-amine |
| 15 | 2-[(3R)-3-({1-[2-(difluoromethoxy)-4-(trifluoromethyl)phenyl]-8-fluoropyrrolo[1,2- |
| | d][1,2,4]triazin-4-y]}amino)piperidin-1-yl]ethan-1-ol |
| 16 | (2S)-1-({1-[2-(difluoromethoxy)-4-(trifluoromethyl)phenyl]-8-fluoropyrrolo[1,2- |
| | d][1,2,4]triazin-4-yl}amino)propan-2-ol |
| 17 | N-[(3R)-1-ethylpiperidin-3-yl]-8-fluoro-1-[4-methoxy-2- |
| | (trifluoromethyl)phenyl]pyrrolo[1,2-d][1,2,4]triazin-4-amine |
| 18 | 2-[(3R)-3-({8-fluoro-1-[4-methoxy-2-(trifluoromethyl)phenyl]pyrrolo[1,2- |
| | d][1,2,4]triazin-4-yl}amino)piperidin-1-yl]ethan-1-ol |
| 19 | 1-[2-fluoro-4-(trifluoromethyl)phenyl]-N-[(3R)-1-methylpiperidin-3-yl]pyrrolo[1,2- |
| | d][1,2,4]triazin-4-amine |
| 20 | N-[(3R)-1-ethylpiperidin-3-yl]-1-[2-fluoro-4-(trifluoromethyl)phenyl]pyrrolo[1,2- |
| | d][1,2,4]triazin-4-amine |
| 21 | 2-[(3R)-3-({1-[2-fluoro-4-(trifluoromethyl)phenyl]pyrrolo[1,2-d][1,2,4]triazin-4- |
| | yl}amino)piperidin-1-yl]ethan-1-ol |
| 22 | 2-[(3R)-3-({1-[2-(difluoromethoxy)-4-(trifluoromethyl)phenyl]pyrrolo[1,2- |
| | d][1,2,4]triazin-4-yl}amino)piperidin-1-yl]propan-1-ol |
| 23 | 1-[2-(difluoromethyl)-4-methylphenyl]-7-methyl-N-[(3R)-1-methylpiperidin-3- |
| | yl]pyrrolo[1,2-d][1,2,4]triazin-4-amine |
| 24 | 1-[4-cyclopropyl-2-(difluoromethoxy)phenyl]-7-methyl-N-[(3R)-1-methylpiperidin-3- |
| | yl]pyrrolo[1,2-d][1,2,4]triazin-4-amine |
| 25 | 1-[2-(difluoromethoxy)-4-methylphenyl]-7-methyl-N-[(3R)-1-methylpiperidin-3- |
| | yl]pyrrolo[1,2-d][1,2,4]triazin-4-amine |
| 26 | 1-(4-methoxy-2-methylphenyl)-N-[(3R)-1-methylpiperidin-3-yl]pyrrolo[1,2- |
| | d][1,2,4]triazin-4-amine |
| 27 | 1-(2-cyclopropyl-4-methoxyphenyl)-N-[(3R)-1-methylpiperidin-3-yl]pyrrolo[1,2- |
| | d][1,2,4]triazin-4-amine |
| 28 | 1-[2,4-bis(trifluoromethyl)phenyl]-8-fluoro-N-[(3R)-1-methylpiperidin-3- |
| | yl]pyrrolo[1,2-d][1,2,4]triazin-4-amine |
| 29 | 1-[2,4-bis(trifluoromethyl)phenyl]-N-[(3R)-1-ethylpiperidin-3-yl]-8- |
| | fluoropyrrolo[1,2-d][1,2,4]triazin-4-amine |
| 30 | 2-[(3R)-3-({1-[2,4-bis(trifluoromethyl)phenyl]-8-fluoropyrrolo[1,2-d][1,2,4]triazin-4- |
| | yl}amino)piperidin-1-yl]ethan-1-ol |
| 31 | 1-[2,4-bis(trifluoromethyl)phenyl]-8-fluoro-N-[(3R,5R)-5-fluoro-1-methylpiperidin- |
| | 3-yl]pyrrolo[1,2-d][1,2,4]triazin-4-amine |
| 32 | 1-[2,4-bis(trifluoromethyl)phenyl]-N-[(3R,5R)-1-ethyl-5-fluoropiperidin-3-yl]-8- |
| | fluoropyrrolo[1,2-d][1,2,4]triazin-4-amine |
| 33 | 1-[2-(difluoromethoxy)-4-methylphenyl]-N-[(3R)-1-methylpiperidin-3-yl]pyrrolo[1,2- |
| | d][1,2,4]triazin-4-amine |
| 34 | 1-[2-(difluoromethoxy)-4-methylphenyl]-N-[(3R)-1-ethylpiperidin-3-yl]pyrrolo[1,2- |
| | d][1,2,4]triazin-4-amine |
| 35 | 1-[2-(difluoromethoxy)-4-(trifluoromethyl)phenyl]-N-[(3R)-piperidin-3- |
| | yl]pyrrolo[1,2-d][1,2,4]triazin-4-amine |
| 36 | N-[(3R)-1-methylpiperidin-3-yl]-1-[4-methyl-2- |
| | (trifluoromethoxy)phenyl]pyrrolo[1,2-d][1,2,4]triazin-4-amine |
| 37 | 1-[4-cyclopropyl-2-(trifluoromethoxy)phenyl]-N-[(3R)-1-methylpiperidin-3- |
| | yl]pyrrolo[1,2-d][1,2,4]triazin-4-amine |
| 38 | (1s,3s)-3-({1-[2,4-bis(trifluoromethyl)phenyl]-8-fluoropyrrolo[1,2-d][1,2,4]triazin-4- |
| | yl}amino)-1-methylcyclobutan-1-ol |
| 39 | 8-fluoro-1-[4-methoxy-2-(trifluoromethoxy)phenyl]-N-[(3R)-1-methylpiperidin-3- |
| | yl]pyrrolo[1,2-d][1,2,4]triazin-4-amine |
| 40 | N-[(3R)-1-methylpiperidin-3-yl]-1-[2-(2,2,2-trifluoroethyl)-4- |
| | (trifluoromethyl)phenyl]pyrrolo[1,2-d][1,2,4]triazin-4-amine |
| 41 | (1s,3s)-1-methyl-3-({1-[2-(2,2,2-trifluoroethyl)-4- |
| | (trifluoromethyl)phenyl]pyrrolo[1,2-d][1,2,4]triazin-4-yl}amino)cyclobutan-1-ol |
| 42 | 1-[4-ethyl-2-(trifluoromethoxy)phenyl]-N-[(3R)-1-methylpiperidin-3-yl]pyrrolo[1,2- |
| | d][1,2,4]triazin-4-amine |
| 43 | 1-[4-ethoxy-2-(trifluoromethoxy)phenyl]-N-[(3R)-1-methylpiperidin-3- |
| | yl]pyrrolo[1,2-d][1,2,4]triazin-4-amine |
| 44 | 1-[4-(difluoromethoxy)-2-(trifluoromethoxy)phenyl]-N-[(3R)-1-methylpiperidin-3- |
| | yl]pyrrolo[1,2-d][1,2,4]triazin-4-amine |
| 45 | 2-[(3R)-3-({1-[2-(difluoromethoxy)-4-methylphenyl]pyrrolo[1,2-d][1,2,4]triazin-4- |
| | yl}amino)piperidin-1-yl]ethan-1-ol |
| 46 | (3S,4S)-3-({1-[2-(difluoromethoxy)-4-(trifluoromethyl)phenyl]pyrrolo[1,2- |
| | d][1,2,4]triazin-4-yl}amino)-1-ethylpiperidin-4-ol |
| 47 | 1-(2-bromo-4-methoxyphenyl)-N-[(3R)-1-methylpiperidin-3-yl]pyrrolo[1,2- |
| | d][1,2,4]triazin-4-amine |
| 48 | (3S,4R)-3-({1-[2-(difluoromethoxy)-4-(trifluoromethyl)phenyl]pyrrolo[1,2- |
| | d][1,2,4]triazin-4-yl}amino)-1-ethylpiperidin-4-ol |
| 49 | 1-[2-(difluoromethoxy)-6-methyl-4-(trifluoromethyl)phenyl]-N-[(3R)-1- |
| | methylpiperidin-3-yl]pyrrolo[1,2-d][1,2,4]triazin-4-amine |
| 50 | N-[(3R)-1-ethylpiperidin-3-yl]-8-fluoro-1-[4-methoxy-2- |
| | (trifluoromethoxy)phenyl]pyrrolo[1,2-d][1,2,4]triazin-4-amine |
| 51 | 2-[(3R)-3-({8-fluoro-1-[4-methoxy-2-(trifluoromethoxy)phenyl]pyrrolo[1,2- |
| | d][1,2,4]triazin-4-yl}amino)piperidin-1-yl]ethan-1-ol |
| 52 | 8-fluoro-N-[(3R,5R)-5-fluoro-1-methylpiperidin-3-yl]-1-[4-methoxy-2- |
| | (trifluoromethoxy)phenyl]pyrrolo[1,2-d][1,2,4]triazin-4-amine |
| 53 | N-[(3R,5R)-1-ethyl-5-fluoropiperidin-3-yl]-8-fluoro-1-[4-methoxy-2- |
| | (trifluoromethoxy)phenyl]pyrrolo[1,2-d][1,2,4]triazin-4-amine |
| 54 | (1s,3s)-3-({8-fluoro-1-[4-methoxy-2-(trifluoromethoxy)phenyl]pyrrolo[1,2- |
| | d][1,2,4]triazin-4-yl}amino)-1-methylcyclobutan-1-ol |
| 55 | 1-[2-(difluoromethoxy)-4-methoxyphenyl]-8-fluoro-N-[(3R)-1-methylpiperidin-3- |
| | yl]pyrrolo[1,2-d][1,2,4]triazin-4-amine |
| 56 | 1-[2-(difluoromethoxy)-4-methoxyphenyl]-N-[(3R)-1-ethylpiperidin-3-yl]-8- |
| | fluoropyrrolo[1,2-d][1,2,4]triazin-4-amine |
| 57 | 2-[(3R)-3-({1-[2-(difluoromethoxy)-4-methoxyphenyl]-8-fluoropyrrolo[1,2- |
| | d][1,2,4]triazin-4-yl}amino)piperidin-1-yl]ethan-1-ol |
| 58 | (1s,3s)-3-({1-[2-(difluoromethoxy)-4-methoxyphenyl]-8-fluoropyrrolo[1,2- |
| | d][1,2,4]triazin-4-yl}amino)-1-methylcyclobutan-1-ol |
| 59 | N-[(3R)-1-ethylpiperidin-3-yl]-1-[4-methoxy-2- |
| | (trifluoromethoxy)phenyl[pyrrolo[1,2-d][1,2,4]triazin-4-amine |
| 60 | N-[(3R,5R)-5-fluoro-1-methylpiperidin-3-yl]-1-[4-methoxy-2- |
| | (trifluoromethoxy)phenyl]pyrrolo[1,2-d][1,2,4]triazin-4-amine |
| 61 | 2-[(3R)-3-({1-[4-methoxy-2-(trifluoromethoxy)phenyl]pyrrolo[1,2-d][1,2,4]triazin-4- |
| | yl}amino)piperidin-1-yl]ethan-1-ol |
| 62 | (1S,3R)-3-({1-[4-methoxy-2-(trifluoromethoxy)phenyl]pyrrolo[1,2-d][1,2,4]triazin-4- |
| | yl}amino)cyclohexan-1-ol |
| 63 | (2R)-1-({1-[2-(difluoromethoxy)-4-(trifluoromethyl)phenyl]pyrrolo[1,2- |
| | d][1,2,4]triazin-4-yl}amino)propan-2-ol |
| 64 | 2-({1-[4-methoxy-2-(trifluoromethoxy)phenyl]pyrrolo[1,2-d][1,2,4]triazin-4- |
| | yl}amino)ethan-1-ol |
| 65 | (2S)-1-({1-[4-methoxy-2-(trifluoromethoxy)phenyl]pyrrolo[1,2-d][1,2,4]triazin-4- |
| | yl}amino)propan-2-ol |
| 66 | 1-({1-[4-methoxy-2-(trifluoromethoxy)phenyl]pyrrolo[1,2-d][1,2,4]triazin-4- |
| | yl}amino)-2-methylpropan-2-ol |
| 67 | 1-[4-methoxy-2-(trifluoromethoxy)phenyl]-N-(oxan-4-yl)pyrrolo[1,2-d][1,2,4]triazin- |
| | 4-amine |
| 68 | 1-[4-methoxy-2-(trifluoromethoxy)phenyl]-N-[(3R)-oxolan-3-yl]pyrrolo[1,2- |
| | d][1,2,4]triazin-4-amine |
| 69 | 1-[4-methoxy-2-(trifluoromethoxy)phenyl]-N-[(1-methyl-1H-imidazol-2- |
| | yl)methyl]pyrrolo[1,2-d][1,2,4]triazin-4-amine |
| 70 | (2R)-1-({1-[4-methoxy-2-(trifluoromethoxy)phenyl]pyrrolo[1,2-d][1,2,4]triazin-4- |
| | yl}amino)propan-2-ol |
| 71 | (1s,3s)-3-({1-[4-methoxy-2-(trifluoromethyl)phenyl]pyrrolo[1,2-d][1,2,4]triazin-4- |
| | yl}amino)-1-methylcyclobutan-1-ol |
| 72 | (1s,3s)-3-{[1-(2-chloro-4-methoxyphenyl)pyrrolo[1,2-d][1,2,4]triazin-4-yl]amino}-1- |
| | methylcyclobutan-1-ol |
| 73 | 1-[4-methoxy-2-(trifluoromethoxy)phenyl]-N-{[(2R)-oxolan-2- |
| | yl]methyl}pyrrolo[1,2-d][1,2,4]triazin-4-amine |
| 74 | 1-[4-methoxy-2-(trifluoromethoxy)phenyl]-N-{[(2S)-1-methylpyrrolidin-2- |
| | yl]methyl}pyrrolo[1,2-d][1,2,4]triazin-4-amine |
| 75 | 1-(2-cyclopropyl-6-fluoro-4-methoxyphenyl)-N-[(3R)-1-methylpiperidin-3- |
| | yl]pyrrolo[1,2-d][1,2,4]triazin-4-amine |
| 76 | 1-[4-methoxy-2-(trifluoromethoxy)phenyl]-N-{[(2S)-oxolan-2-yl]methyl}pyrrolo[1,2- |
| | d][1,2,4]triazin-4-amine |
| 77 | 3-({1-[2-(2,2,2-trifluoroethyl)-4-(trifluoromethyl)phenyl]pyrrolo[1,2-d][1,2,4]triazin- |
| | 4-yl}amino)pyrrolidin-2-one |
| 78 | (1S,2S)-2-({1-[2-(difluoromethoxy)-4-(trifluoromethyl)phenyl]pyrrolo[1,2- |
| | d][1,2,4]triazin-4-yl}amino)cyclohexan-1-ol |
| 79 | (1R,2S)-2-({1-[2-(difluoromethoxy)-4-(trifluoromethyl)phenyl]pyrrolo[1,2- |
| | d][1,2,4]triazin-4-yl}amino)cyclohexan-1-ol |
| 80 | (1S,2S)-2-({1-[2-(difluoromethoxy)-4-methylphenyl]pyrrolo[1,2-d][1,2,4]triazin-4- |
| | yl}amino)cyclohexan-1-ol |
| 81 | (1S,2S)-2-({1-[4-methoxy-2-(trifluoromethoxy)phenyl]pyrrolo[1,2-d][1,2,4]triazin-4- |
| | yl}amino)cyclohexan-1-ol |
| 82 | 1-(2-chloro-4-methoxyphenyl)-N-[(3R)-1-ethylpiperidin-3-yl]-8-fluoropyrrolo[1,2- |
| | d][1,2,4]triazin-4-amine |
| 83 | 2-[(3R)-3-{[1-(2-chloro-4-methoxyphenyl)-8-fluoropyrrolo[1,2-d][1,2,4]triazin-4- |
| | yl]amino}piperidin-1-yl]ethan-1-ol |
| 84 | (1s,3s)-3-{[1-(2-chloro-4-methoxyphenyl)-8-fluoropyrrolo[1,2-d][1,2,4]triazin-4- |
| | yl]amino}-1-methylcyclobutan-1-ol |
| 85 | 1-[4-(difluoromethoxy)-2-(trifluoromethoxy)phenyl]-N-[(3R)-1-ethylpiperidin-3- |
| | yl]pyrrolo[1,2-d][1,2,4]triazin-4-amine |
| 86 | 2-[(3R)-3-({1-[4-(difluoromethoxy)-2-(trifluoromethoxy)phenyl]pyrrolo[1,2- |
| | d][1,2,4]triazin-4-yl}amino)piperidin-1-yl]ethan-1-ol |
| 87 | 1-[4-(difluoromethoxy)-2-(trifluoromethoxy)phenyl]-N-[(3R,5R)-5-fluoro-1- |
| | methylpiperidin-3-yl]pyrrolo[1,2-d][1,2,4]triazin-4-amine |
| 88 | (1s,3s)-3-({1-[4-(difluoromethoxy)-2-(trifluoromethoxy)phenyl]pyrrolo[1,2- |
| | d][1,2,4]triazin-4-yl}amino)-1-methylcyclobutan-1-ol |
| 89 | 3-[1-({1-[2-(difluoromethoxy)-4-(trifluoromethyl)phenyl]pyrrolo[1,2-d][1,2,4]triazin- |
| | 4-yl}amino)ethyl]phenol |
| 90 | (1R,2R)-2-({1-[2-(2,2,2-trifluoroethyl)-4-(trifluoromethyl)phenyl]pyrrolo[1,2- |
| | d][1,2,4]triazin-4-yl}amino)cyclohexan-1-ol |
| 91 | 1-[4-methoxy-2-(trifluoromethoxy)phenyl]-N-[(1,3-oxazol-2-yl)methyl]pyrrolo[1,2- |
| | d][1,2,4]triazin-4-amine |
| 92 | 1-[4-methoxy-2-(trifluoromethoxy)phenyl]-N-[(4-methyl-4H-1,2,4-triazol-3- |
| | yl)methyl]pyrrolo[1,2-d][1,2,4]triazin-4-amine |
| 93 | 1-[4-methoxy-2-(trifluoromethoxy)phenyl]-N-[(1-methyl-1H-1,2,4-triazol-5- |
| | yl)methyl]pyrrolo[1,2-d][1,2,4]triazin-4-amine |
| 94 | [3-({1-[4-methoxy-2-(trifluoromethoxy)phenyl]pyrrolo[1,2-d][1,2,4]triazin-4- |
| | yl}amino)bicyclo[1.1.1]pentan-1-yl]methanol |
| 95 | 1-[3-(difluoromethoxy)-4-(4-{[(3R)-1-methylpiperidin-3-yl]amino}pyrrolo[1,2- |
| | d][1,2,4]triazin-1-yl)phenyl]propan-1-one |
| 96 | 3-[1-({1-[2-(2,2,2-trifluoroethyl)-4-(trifluoromethyl)phenyl]pyrrolo[1,2- |
| | d][1,2,4]triazin-4-yl}amino)ethyl]phenol |
| 97 | N-(1H-pyrazol-3-yl)-1-[2-(2,2,2-trifluoroethyl)-4- |
| | (trifluoromethyl)phenyl]pyrrolo[1,2-d][1,2,4]triazin-4-amine |
| 98 | (3S,5R)-5-({1-[2-(difluoromethoxy)-4-(trifluoromethyl)phenyl]pyrrolo[1,2- |
| | d][1,2,4]triazin-4-yl}amino)-1-methylpiperidin-3-ol |
| 99 | (1S,2S)-2-({1-[2,4-bis(trifluoromethyl)phenyl]pyrrolo[1,2-d][1,2,4]triazin-4- |
| | yl}amino)cyclohexan-1-ol |
| 100 | 3-[1-({1-[2-(difluoromethoxy)-4-methylphenyl]pyrrolo[1,2-d][1,2,4]triazin-4- |
| | yl}amino)ethyl]phenol |
| 101 | 3-[{{1-[2-(difluoromethoxy)-4-(trifluoromethyl)phenyl]pyrrolo[1,2-d][1,2,4]triazin-4- |
| | yl}amino)methyl]phenol |
| 102 | 3-[(1R)-1-({1-[2-(difluoromethoxy)-4-(trifluoromethyl)phenyl]pyrrolo[1,2- |
| | d][1,2,4]triazin-4-yl}amino)ethyl]phenol OR 3-[(1S)-1-({1-[2-(difluoromethoxy)-4- |
| | (trifluoromethyl)phenyl]pyrrolo[1,2-d][1,2,4]triazin-4-yl}amino)ethyl]phenol |
| 103 | 3-[(1S)-1-({1-[2-(difluoromethoxy)-4-(trifluoromethyl)phenyl]pyrrolo[1,2- |
| | d][1,2,4]triazin-4-yl}amino)ethyl]phenol OR 3-[(1R)-1-({1-[2-(difluoromethoxy)-4- |
| | (trifluoromethyl)phenyl]pyrrolo[1,2-d][1,2,4]triazin-4-yl}amino)ethyl]phenol |
| 104 | (1s,3s)-3-{[1-(2-cyclopropyl-4-methoxyphenyl)pyrrolo[1,2-d][1,2,4]triazin-4- |
| | yl]amino}-1-methylcyclobutan-1-ol |
| 105 | 1-(2-cyclopropyl-4-methoxyphenyl)-N-(1H-pyrazol-3-yl)pyrrolo[1,2-d][1,2,4]triazin- |
| | 4-amine |
| 106 | (1S,3R)-3-{[1-(2-cyclopropyl-4-methoxyphenyl)pyrrolo[1,2-d][1,2,4]triazin-4- |
| | yl]amino}cyclohexan-1-ol |
| 107 | (1s,3s)-3-({1-[2,4-bis(trifluoromethyl)phenyl]-7-methylpyrrolo[1,2-d][1,2,4]triazin-4- |
| | yl}amino)-1-methylcyclobutan-1-ol |
| 108 | (1s,3s)-3-({1-[4-methoxy-2-(trifluoromethoxy)phenyl]-7-methylpyrrolo[1,2- |
| | d][1,2,4]triazin-4-yl}amino)-1-methylcyclobutan-1-ol |
| 109 | 1-[2,4-bis(trifluoromethyl)phenyl]-7-methyl-N-[(3R)-1-methylpiperidin-3- |
| | yl]pyrrolo[1,2-d][1,2,4]triazin-4-amine |
| 110 | (1R,2R)-2-({1-[2-(difluoromethoxy)-4-(trifluoromethyl)phenyl]pyrrolo[1,2- |
| | d][1,2,4]triazin-4-yl}amino)cyclohexan-1-ol |
| 111 | (1S,2S)-2-({1-[2-(2,2,2-trifluoroethyl)-4-(trifluoromethyl)phenyl]pyrrolo[1,2- |
| | d][1,2,4]triazin-4-yl}amino)cyclohexan-1-ol |
| 112 | (3S,4R)-4-({1-[2-(2,2,2-trifluoroethyl)-4-(trifluoromethyl)phenyl]pyrrolo[1,2- |
| | d][1,2,4]triazin-4-yl}amino)oxan-3-ol |
| 113 | (3R,4S)-4-({1-[2-(2,2,2-trifluoroethyl)-4-(trifluoromethyl)phenyl]pyrrolo[1,2- |
| | d][1,2,4]triazin-4-yl}amino)oxan-3-ol |
| 114 | (3R,4R)-4-({1-[2-(2,2,2-trifluoroethyl)-4-(trifluoromethyl)phenyl]pyrrolo[1,2- |
| | d][1,2,4]triazin-4-yl}amino)oxan-3-ol |
| 115 | (3S,4S)-4-({1-[2-(2,2,2-trifluoroethyl)-4-(trifluoromethyl)phenyl]pyrrolo[1,2- |
| | d][1,2,4]triazin-4-yl}amino)oxan-3-ol |
| 116 | 3-({8-fluoro-1-[4-methoxy-2-(trifluoromethyl)phenyl]pyrrolo[1,2-d][1,2,4]triazin-4- |
| | yl}amino)pyrrolidin-2-one |
| 117 | 8-fluoro-1-[4-methoxy-2-(trifluoromethyl)phenyl]-N-(1H-pyrazol-3-yl)pyrrolo[1,2- |
| | d][1,2,4]triazin-4-amine |
| 118 | 1-(2-fluoro-4-methoxy-6-methylphenyl)-N-[(3R)-1-methylpiperidin-3-yl]pyrrolo[1,2- |
| | d][1,2,4]triazin-4-amine |
| 119 | N-[(3R)-1-ethylpiperidin-3-yl]-1-[2-(trifluoromethoxy)-4- |
| | (trifluoromethyl)phenyl]pyrrolo[1,2-d][1,2,4]triazin-4-amine |
| 120 | 2-[(3R)-3-({1-[2-(trifluoromethoxy)-4-(trifluoromethyl)phenyl]pyrrolo[1,2- |
| | d][1,2,4]triazin-4-yl}amino)piperidin-1-yl]ethan-1-ol |
| 121 | (1S,2S)-2-({1-[2-(trifluoromethoxy)-4-(trifluoromethyl)phenyl]pyrrolo[1,2- |
| | d][1,2,4]triazin-4-yl}amino)cyclohexan-1-ol |
| 122 | (1S,3R)-3-({1-[2-(trifluoromethoxy)-4-(trifluoromethyl)phenyl]pyrrolo[1,2- |
| | d][1,2,4]triazin-4-yl}amino)cyclohexan-1-ol |
| 123 | (1s,3s)-1-methyl-3-({1-[2-(trifluoromethoxy)-4-(trifluoromethyl)phenyl]pyrrolo[1,2- |
| | d][1,2,4]triazin-4-yl}amino)cyclobutan-1-ol |
| 124 | 3-({1-[2-(trifluoromethoxy)-4-(trifluoromethyl)phenyl]pyrrolo[1,2-d][1,2,4]triazin-4- |
| | yl}amino)pyrrolidin-2-one |
| 125 | 1-[4-methoxy-2-(trifluoromethoxy)phenyl]-N-[(1-methyl-1H-tetrazol-5- |
| | yl)methyl]pyrrolo[1,2-d][1,2,4]triazin-4-amine |
| 126 | (1S,2R)-2-({1-[2-(2,2,2-trifluoroethyl)-4-(trifluoromethyl)phenyl]pyrrolo[1,2- |
| | d][1,2,4]triazin-4-yl}amino)cycloheptan-1-ol AND (1R,2S)-2-({1-[2-(2,2,2- |
| | trifluoroethyl)-4-(trifluoromethyl)phenyl]pyrrolo[1,2-d][1,2,4]triazin-4- |
| | yl}amino)cycloheptan-1-ol |
| 127 | (1R,2R)-2-({1-[2-(2,2,2-trifluoroethyl)-4-(trifluoromethyl)phenyl]pyrrolo[1,2- |
| | d][1,2,4]triazin-4-yl}amino)cycloheptan-1-ol AND (1S,2S)-2-({1-[2-(2,2,2- |
| | trifluoroethyl)-4-(trifluoromethyl)phenyl]pyrrolo[1,2-d][1,2,4]triazin-4- |
| | yl}amino)cycloheptan-1-ol |
| 128 | (1S,3R)-3-({1-[2-(2,2,2-trifluoroethyl)-4-(trifluoromethyl)phenyl]pyrrolo[1,2- |
| | d][1,2,4]triazin-4-yl}amino)cycloheptan-1-ol AND (1R,3S)-3-({1-[2-(2,2,2- |
| | trifluoroethyl)-4-(trifluoromethyl)phenyl]pyrrolo[1,2-d][1,2,4]triazin-4- |
| | yl}amino)cycloheptan-1-ol |
| 129 | (1R,3R)-3-({1-[2-(2,2,2-trifluoroethyl)-4-(trifluoromethyl)phenyl]pyrrolo[1,2- |
| | d][1,2,4]triazin-4-yl}amino)cycloheptan-1-ol AND (1S,3S)-3-({1-[2-(2,2,2- |
| | trifluoroethyl)-4-(trifluoromethyl)phenyl]pyrrolo[1,2-d][1,2,4]triazin-4- |
| | yl}amino)cycloheptan-1-ol |
| 130 | (1s,3s)-3-({8-fluoro-1-[4-methoxy-2-(trifluoromethyl)phenyl]pyrrolo[1,2- |
| | d][1,2,4]triazin-4-yl}amino)-1-methylcyclobutan-1-ol |
| 131 | 1-[4-methoxy-2-(trifluoromethoxy)phenyl]-7-methyl-N-[(3R)-1-methylpiperidin-3- |
| | yl]pyrrolo[1,2-d][1,2,4]triazin-4-amine |
| 132 | (1S,2S)-2-({1-[2,4-bis(trifluoromethyl)phenyl]-7-methylpyrrolo[1,2-d][1,2,4]triazin- |
| | 4-yl}amino)cyclohexan-1-ol |
| 133 | (1S,2S)-2-({1-[4-methoxy-2-(trifluoromethoxy)phenyl]-7-methylpyrrolo[1,2- |
| | d][1,2,4]triazin-4-yl}amino)cyclohexan-1-ol |
| 134 | (3S,4R)-3-({1-[2,4-bis(trifluoromethyl)phenyl]-7-methylpyrrolo[1,2-d][1,2,4]triazin- |
| | 4-yl}amino)-1-ethylpiperidin-4-ol |
| 135 | (3S,4R)-1-ethyl-3-({1-[4-methoxy-2-(trifluoromethoxy)phenyl]-7-methylpyrrolo[1,2- |
| | d][1,2,4]triazin-4-yl}amino)piperidin-4-ol |
| 136 | (1R,2R)-2-({1-[2-(difluoromethoxy)-4-methylphenyl]pyrrolo[1,2-d][1,2,4]triazin-4- |
| | yl}amino)cyclohexan-1-ol |
| 137 | (1S,2R)-2-({1-[2-(difluoromethoxy)-4-methylphenyl]pyrrolo[1,2-d][1,2,4]triazin-4- |
| | yl}amino)cyclohexan-1-ol |
| 138 | (1R,2R)-2-({1-[4-methoxy-2-(2,2,2-trifluoroethyl)phenyl]pyrrolo[1,2-d][1,2,4]triazin- |
| | 4-yl}amino)cyclohexan-1-ol |
| 139 | 3-({1-[4-methoxy-2-(2,2,2-trifluoroethyl)phenyl]pyrrolo[1,2-d][1,2,4]triazin-4- |
| | yl}amino)pyrrolidin-2-one |
| 140 | N-[(3R)-1-methylpiperidin-3-yl]-1-[2-(trifluoromethoxy)-4- |
| | (trifluoromethyl)phenyl]pyrrolo[1,2-d][1,2,4]triazin-4-amine |
| 141 | [(1s,3s)-3-({1-[2-(trifluoromethoxy)-4-(trifluoromethyl)phenyl]pyrrolo[1,2- |
| | d][1,2,4]triazin-4-yl}amino)cyclobutyl]methanol |
| 142 | 3-[1-({1-[2-(trifluoromethoxy)-4-(trifluoromethyl)phenyl]pyrrolo[1,2-d][1,2,4]triazin- |
| | 4-yl}amino)ethyl]phenol |
| 143 | 2-[(3R)-3-({1-[2-(cyclopropyloxy)-4-methoxyphenyl]pyrrolo[1,2-d][1,2,4]triazin-4- |
| | yl}amino)piperidin-1-yl]ethan-1-ol |
| 144 | (3S,4R)-4-({1-[4-methoxy-2-(2,2,2-trifluoroethyl)phenyl]pyrrolo[1,2-d][1,2,4]triazin- |
| | 4-yl}amino)oxan-3-ol |
| 145 | 1-[4-methoxy-2-(2,2,2-trifluoroethyl)phenyl]-N-[(3R)-1-methylpiperidin-3- |
| | yl]pyrrolo[1,2-d][1,2,4]triazin-4-amine |
| 146 | N-{[(3S)-1-methylpiperidin-3-yl]methyl}-1-[2-(2,2,2-trifluoroethyl)-4- |
| | (trifluoromethyl)phenyl]pyrrolo[1,2-d][1,2,4]triazin-4-amine |
| 147 | N-{[(3R)-1-methylpiperidin-3-yl]methyl}-1-[2-(2,2,2-trifluoroethyl)-4- |
| | (trifluoromethyl)phenyl]pyrrolo[1,2-d][1,2,4]triazin-4-amine |
| 148 | 8-fluoro-N-[1-(3-methoxyphenyl)ethyl]-1-[4-methoxy-2- |
| | (trifluoromethyl)phenyl]pyrrolo[1,2-d][1,2,4]triazin-4-amine |
| 149 | 3-[1-({8-fluoro-1-[4-methoxy-2-(trifluoromethyl)phenyl]pyrrolo[1,2-d][1,2,4]triazin- |
| | 4-yl}amino)ethyl]phenol |
| 150 | N-(1H-pyrazol-3-yl)-1-[2-(trifluoromethoxy)-4-(trifluoromethyl)phenyl]pyrrolo[1,2- |
| | d][1,2,4]triazin-4-amine |
| 151 | (1R,2R)-2-({1-[2-(trifluoromethoxy)-4-(trifluoromethyl)phenyl]pyrrolo[1,2- |
| | d][1,2,4]triazin-4-yl}amino)cyclohexan-1-ol |
| 152 | 1-[4-(difluoromethoxy)-2-(trifluoromethoxy)phenyl]-N-[(3R)-oxan-3-yl]pyrrolo[1,2- |
| | d][1,2,4]triazin-4-amine |
| 153 | (1S,3R)-3-({1-[4-(difluoromethoxy)-2-(trifluoromethoxy)phenyl]pyrrolo[1,2- |
| | d][1,2,4]triazin-4-yl}amino)cyclohexan-1-ol |
| 154 | 3-({1-[4-(difluoromethoxy)-2-(trifluoromethoxy)phenyl]pyrrolo[1,2-d][1,2,4]triazin- |
| | 4-yl}amino)pyrrolidin-2-one |
| 155 | 1-[4-(difluoromethoxy)-2-(trifluoromethoxy)phenyl]-N-(1H-pyrazol-3-yl)pyrrolo[1,2- |
| | d][1,2,4]triazin-4-amine |
| 156 | (1R,2R)-2-({1-[4-methyl-2-(2,2,2-trifluoroethyl)phenyl]pyrrolo[1,2-d][1,2,4]triazin-4- |
| | yl}amino)cyclohexan-1-ol |
| 157 | 3-({1-[4-methyl-2-(2,2,2-trifluoroethyl)phenyl]pyrrolo[1,2-d][1,2,4]triazin-4- |
| | yl}amino)pyrrolidin-2-one |
| 158 | (1s,3s)-1-methyl-3-({1-[4-methyl-2-(2,2,2-trifluoroethyl)phenyl]pyrrolo[1,2- |
| | d][1,2,4]triazin-4-yl}amino)cyclobutan-1-ol |
| 159 | N-[(3R)-1-methylpiperidin-3-yl]-1-[4-methyl-2-(2,2,2- |
| | trifluoroethyl)phenyl]pyrrolo[1,2-d][1,2,4]triazin-4-amine |
| 160 | (1R,2R)-2-({1-[2,4-bis(trifluoromethyl)phenyl]-7-methylpyrrolo[1,2-d][1,2,4]triazin- |
| | 4-yl}amino)cyclohexan-1-ol |
| 161 | 3-({1-[2,4-bis(trifluoromethyl)phenyl]-7-methylpyrrolo[1,2-d][1,2,4]triazin-4- |
| | yl}amino)pyrrolidin-2-one |
| 162 | 3-({1-[4-methoxy-2-(trifluoromethoxy)phenyl]-7-methylpyrrolo[1,2-d][1,2,4]triazin- |
| | 4-yl}amino)pyrrolidin-2-one |
| 163 | (1R,2R)-2-({1-[4-methoxy-2-(trifluoromethoxy)phenyl]-7-methylpyrrolo[1,2- |
| | d][1,2,4]triazin-4-yl}amino)cyclohexan-1-ol |
| 164 | (3S,4R)-4-({1-[2,4-bis(trifluoromethyl)phenyl]-7-methylpyrrolo[1,2-d][1,2,4]triazin- |
| | 4-yl}amino)oxan-3-ol |
| 165 | (3S,4R)-4-({1-[4-methoxy-2-(trifluoromethoxy)phenyl]-7-methylpyrrolo[1,2- |
| | d][1,2,4]triazin-4-yl}amino)oxan-3-ol |
| 166 | 1-[2-(difluoromethoxy)-4-methoxyphenyl]-N-[(3R)-1-ethylpiperidin-3-yl]pyrrolo[1,2- |
| | d][1,2,4]triazin-4-amine |
| 167 | (3S,4R)-4-({1-[2-(difluoromethoxy)-4-methoxyphenyl]pyrrolo[1,2-d][1,2,4]triazin-4- |
| | yl}amino)oxan-3-ol |
| 168 | (1R,2R)-2-({1-[4-(difluoromethoxy)-2-(trifluoromethoxy)phenyl]pyrrolo[1,2- |
| | d][1,2,4]triazin-4-yl}amino)cyclohexan-1-ol |
| 169 | N-[(3R)-1-ethylpiperidin-3-yl]-1-[2-(fluoromethoxy)-4- |
| | (trifluoromethyl)phenyl]pyrrolo[1,2-d][1,2,4]triazin-4-amine |
| 170 | (3S,4R)-3-({1-[4-(difluoromethoxy)-2-(trifluoromethoxy)phenyl]pyrrolo[1,2- |
| | d][1,2,4]triazin-4-yl}amino)-1-methylpiperidin-4-ol |
| 171 | (3S,4R)-3-({1-[4-(difluoromethoxy)-2-(trifluoromethoxy)phenyl]pyrrolo[1,2- |
| | d][1,2,4]triazin-4-yl}amino)-1-ethylpiperidin-4-ol |
| 172 | 3-[1-({1-[4-(difluoromethoxy)-2-(trifluoromethoxy)phenyl]pyrrolo[1,2- |
| | d][1,2,4]triazin-4-yl}amino)ethyl]phenol |
| 173 | 2-[(3R)-3-({1-[2-(fluoromethoxy)-4-(trifluoromethyl)phenyl]pyrrolo[1,2- |
| | d][1,2,4]triazin-4-yl}amino)piperidin-1-yl]ethan-1-ol |
| 174 | 3-({1-[2-(difluoromethoxy)-4-methoxyphenyl]pyrrolo[1,2-d][1,2,4]triazin-4- |
| | yl}amino)pyrrolidin-2-one |
| 175 | (3S,4R)-3-({1-[2-(difluoromethoxy)-4-methoxyphenyl]pyrrolo[1,2-d][1,2,4]triazin-4- |
| | yl}amino)-1-ethylpiperidin-4-ol |
| 176 | [(1R,3S)-3-({1-[2-(difluoromethoxy)-4-methoxyphenyl]pyrrolo[1,2-d][1,2,4]triazin- |
| | 4-yl}amino)cyclohexyl]methanol |
| 177 | (1R,2R)-2-({1-[2-(difluoromethoxy)-4-methoxyphenyl]pyrrolo[1,2-d][1,2,4]triazin-4- |
| | yl}amino)cyclohexan-1-ol |
| 178 | 1-[2-(cyclopropyloxy)-4-(trifluoromethyl)phenyl]-N-[(3R)-1-ethylpiperidin-3- |
| | yl]pyrrolo[1,2-d][1,2,4]triazin-4-amine |
| 179 | 2-[(3R)-3-({1-[2-(cyclopropyloxy)-4-(trifluoromethyl)phenyl]pyrrolo[1,2- |
| | d][1,2,4]triazin-4-yl}amino)piperidin-1-yl]ethan-1-ol |
| 180 | (1S,2S)-2-({1-[4-methoxy-2-(trifluoromethoxy)phenyl]pyrrolo[1,2-d][1,2,4]triazin-4- |
| | yl}amino)-1-methylcyclohexan-1-ol OR (1R,2R)-2-({1-[4-methoxy-2- |
| | (trifluoromethoxy)phenyl]pyrrolo[1,2-d][1,2,4]triazin-4-yl}amino)-1- |
| | methylcyclohexan-1-ol |
| 181 | (1R,2R)-2-({1-[4-methoxy-2-(trifluoromethoxy)phenyl]pyrrolo[1,2-d][1,2,4]triazin-4- |
| | yl}amino)-1-methylcyclohexan-1-ol OR (1S,2S)-2-({1-[4-methoxy-2- |
| | (trifluoromethoxy)phenyl]pyrrolo[1,2-d][1,2,4]triazin-4-yl}amino)-1- |
| | methylcyclohexan-1-ol |
| 182 | 1-[2-(cyclopropyloxy)-4-methoxyphenyl]-N-[(3R)-1-ethylpiperidin-3-yl]pyrrolo[1,2- |
| | d][1,2,4]triazin-4-amine |
| 183 | [(1s,3s)-3-({1-[2-(difluoromethoxy)-4-methylphenyl]imidazo[1,5-d][1,2,4]triazin-4- |
| | yl}amino)cyclobutyl]methanol |
| 184 | 2-[(3R)-3-({1-[2-(difluoromethoxy)-4-methylphenyl]imidazo[1,5-d][1,2,4]triazin-4- |
| | yl}amino)piperidin-1-yl]ethan-1-ol |
| 185 | 2-[(3R)-3-({1-[4-cyclopropyl-2-(difluoromethoxy)phenyl]imidazo[1,5- |
| | d][1,2,4]triazin-4-yl}amino)piperidin-1-yl]ethan-1-ol |
| 186 | 2-[(3R)-3-({1-[2-(difluoromethoxy)-4-(trifluoromethyl)phenyl]imidazo[1,5- |
| | d][1,2,4]triazin-4-yl}amino)piperidin-1-yl]ethan-1-ol |
| 187 | (1s,3s)-3-({1-[2-(difluoromethoxy)-4-(trifluoromethyl)phenyl]imidazo[1,5- |
| | d][1,2,4]triazin-4-yl}amino)-1-methylcyclobutan-1-ol |
| 188 | 1-[2-(difluoromethoxy)-4-(trifluoromethyl)phenyl]-N-[(3R)-oxan-3-yl]imidazo[1,5- |
| | d][1,2,4]triazin-4-amine |
| 189 | 1-[4-chloro-2-(difluoromethoxy)phenyl]-N-[(3R)-oxan-3-yl]imidazo[1,5- |
| | d][1,2,4]triazin-4-amine |
| 190 | 1-[4-chloro-2-(difluoromethoxy)phenyl]-N-[(3R)-1-methylpiperidin-3- |
| | yl]imidazo[1,5-d][1,2,4]triazin-4-amine |
| 191 | 1-[4-chloro-2-(difluoromethoxy)phenyl]-N-[(3R)-1-ethylpiperidin-3-yl]imidazo[1,5- |
| | d][1,2,4]triazin-4-amine |
| 192 | 2-[(3R)-3-({1-[4-chloro-2-(difluoromethoxy)phenyl]imidazo[1,5-d][1,2,4]triazin-4- |
| | yl}amino)piperidin-1-yl]ethan-1-ol |
| 193 | 1-[4-chloro-2-(difluoromethoxy)phenyl]-N-[(3R,5R)-5-fluoro-1-methylpiperidin-3- |
| | yl]imidazo[1,5-d][1,2,4]triazin-4-amine |
| 194 | (1s,3s)-3-({1-[4-chloro-2-(difluoromethoxy)phenyl]imidazo[1,5-d][1,2,4]triazin-4- |
| | yl}amino)-1-methylcyclobutan-1-ol |
| 195 | 1-[4-bromo-2-(difluoromethoxy)phenyl]-N-[(3R)-1-methylpiperidin-3- |
| | yl]imidazo[1,5-d][1,2,4]triazin-4-amine |
| 196 | 1-[4-bromo-2-(difluoromethoxy)phenyl]-N-[(3R,5R)-1-ethyl-5-fluoropiperidin-3- |
| | yl]imidazo[1,5-d][1,2,4]triazin-4-amine |
| 197 | N-[(3R)-1-methylpiperidin-3-yl]-1-[2-(trifluoromethoxy)-4- |
| | (trifluoromethyl)phenyl]imidazo[1,5-d][1,2,4]triazin-4-amine |
| 198 | 2-[(3R)-3-({1-[4-bromo-2-(difluoromethoxy)phenyl]imidazo[1,5-d][1,2,4]triazin-4- |
| | yl}amino)piperidin-1-yl]ethan-1-ol |
| 199 | (1s,3s)-3-({1-[4-bromo-2-(difluoromethoxy)phenyl]imidazo[1,5-d][1,2,4]triazin-4- |
| | yl}amino)-1-methylcyclobutan-1-ol |
| 200 | 1-[4-bromo-2-(trifluoromethyl)phenyl]-N-[(3R)-1-methylpiperidin-3-yl]imidazo[1,5- |
| | d][1,2,4]triazin-4-amine |
| 201 | 2-[(3R)-3-({1-[4-bromo-2-(trifluoromethyl)phenyl]imidazo[1,5-d][1,2,4]triazin-4- |
| | yl}amino)piperidin-1-yl]ethan-1-ol |
| 202 | 1-[2-(difluoromethoxy)-4-methylphenyl]-N-[(3R)-1-methylpiperidin-3- |
| | yl]imidazo[1,5-d][1,2,4]triazin-4-amine |
| 203 | 1-[4-cyclopropyl-2-(difluoromethoxy)phenyl]-N-[(3R)-1-methylpiperidin-3- |
| | yl]imidazo[1,5-d][1,2,4]triazin-4-amine |
| 204 | (1s,3s)-3-({1-[4-bromo-2-(trifluoromethyl)phenyl]imidazo[1,5-d][1,2,4]triazin-4- |
| | yl}amino)-1-methylcyclobutan-1-ol |
| 205 | 1-[4-bromo-2-(trifluoromethyl)phenyl]-N-[(3R,5R)-1-ethyl-5-fluoropiperidin-3- |
| | yl]imidazo[1,5-d][1,2,4]triazin-4-amine |
| 206 | N-[(3R)-1-methylpiperidin-3-yl]-1-[4-methyl-2-(trifluoromethyl)phenyl]imidazo[1,5- |
| | d][1,2,4]triazin-4-amine |
| 207 | 1-[4-cyclopropyl-2-(trifluoromethyl)phenyl]-N-[(3R)-1-methylpiperidin-3- |
| | yl]imidazo[1,5-d][1,2,4]triazin-4-amine |
| 208 | 1-[4-methoxy-2-(trifluoromethoxy)phenyl]-N-[(3R)-1-methylpiperidin-3- |
| | yl]imidazo[1,5-d][1,2,4]triazin-4-amine |
| 209 | N-[(3R)-1-ethylpiperidin-3-yl]-1-[4-methoxy-2- |
| | (trifluoromethoxy)phenyl]imidazo[1,5-d][1,2,4]triazin-4-amine |
| 210 | N-[(3R,5R)-5-fluoro-1-methylpiperidin-3-yl]-1-[4-methoxy-2- |
| | (trifluoromethoxy)phenyl]imidazo[1,5-d][1,2,4]triazin-4-amine |
| 211 | 2-[(3R)-3-({1-[4-methoxy-2-(trifluoromethoxy)phenyl]imidazo[1,5-d][1,2,4]triazin-4- |
| | yl}amino)piperidin-1-yl]ethan-1-ol |
| 212 | N-[(3R)-1-ethylpiperidin-3-yl]-1-[4-methoxy-2-(trifluoromethyl)phenyl]imidazo[1,5- |
| | d][1,2,4]triazin-4-amine |
| 213 | 2-[(3R)-3-({1-[4-methoxy-2-(trifluoromethyl)phenyl]imidazo[1,5-d][1,2,4]triazin-4- |
| | yl}amino)piperidin-1-yl]ethan-1-ol |
| 214 | (1s,3s)-3-({1-[4-methoxy-2-(trifluoromethyl)phenyl]imidazo[1,5-d][1,2,4]triazin-4- |
| | yl}amino)-1-methylcyclobutan-1-ol |
| 215 | (1S,3R)-3-({1-[2,4-bis(trifluoromethyl)phenyl]imidazo[1,5-d][1,2,4]triazin-4- |
| | yl}amino)cyclohexan-1-ol |
| 216 | 1-[2-(difluoromethoxy)-4-methoxyphenyl]-N-[(3R)-1-methylpiperidin-3- |
| | yl]imidazo[1,5-d][1,2,4]triazin-4-amine |
| 217 | 1-[2,4-bis(trifluoromethyl)phenyl]-N-[(3R)-1-methylpiperidin-3-yl]imidazo[1,5- |
| | d][1,2,4]triazin-4-amine |
| 218 | (1s,3s)-3-({1-[2,4-bis(trifluoromethyl)phenyl]imidazo[1,5-d][1,2,4]triazin-4- |
| | yl}amino)-1-methylcyclobutan-1-ol |
| 219 | (1s,3s)-3-({1-[4-methoxy-2-(trifluoromethoxy)phenyl]imidazo[1,5-d][1,2,4]triazin-4- |
| | yl}amino)-1-methylcyclobutan-1-ol |
| 220 | 1-[4-methoxy-2-(trifluoromethoxy)phenyl]-N-(1H-pyrazol-3-yl)imidazo[1,5- |
| | d][1,2,4]triazin-4-amine |
| 221 | (1R,2R)-2-({1-[4-methoxy-2-(trifluoromethoxy)phenyl]imidazo[1,5-d][1,2,4]triazin- |
| | 4-yl}amino)cyclopentan-1-ol |
| 222 | (1S,3R)-3-({1-[4-methoxy-2-(trifluoromethoxy)phenyl]imidazo[1,5-d][1,2,4]triazin- |
| | 4-yl}amino)cyclohexan-1-ol |
| 223 | (1R,2R)-2-({1-[4-methoxy-2-(trifluoromethoxy)phenyl]imidazo[1,5-d][1,2,4]triazin- |
| | 4-yl}amino)cyclohexan-1-ol |
| 224 | 1-[2-(difluoromethoxy)-4-methoxyphenyl]-N-[(3R,5R)-5-fluoro-1-methylpiperidin-3- |
| | yl]imidazo[1,5-d][1,2,4]triazin-4-amine |
| 225 | 2-[(3R)-3-({1-[2-(difluoromethoxy)-4-methoxyphenyl]imidazo[1,5-d][1,2,4]triazin-4- |
| | yl}amino)piperidin-1-yl]ethan-1-ol |
| 226 | (1s,3s)-3-({1-[2-(difluoromethoxy)-4-methoxyphenyl]imidazo[1,5-d][1,2,4]triazin-4- |
| | yl}amino)-1-methylcyclobutan-1-ol |
| 227 | 1-[2-(difluoromethoxy)-4-methoxyphenyl]-N-(1H-pyrazol-3-yl)imidazo[1,5- |
| | d][1,2,4]triazin-4-amine |
| 228 | 1-[4-methoxy-2-(trifluoromethyl)phenyl]-N-(1H-pyrazol-3-yl)imidazo[1,5- |
| | d][1,2,4]triazin-4-amine |
| 229 | N-[(3R)-1-ethylpiperidin-3-yl]-1-[2-(trifluoromethoxy)-4- |
| | (trifluoromethyl)phenyl]imidazo[1,5-d][1,2,4]triazin-4-amine |
| 230 | N-[1-(3-methoxyphenyl)ethyl]-1-[4-methoxy-2- |
| | (trifluoromethoxy)phenyl]imidazo[1,5-d][1,2,4]triazin-4-amine |
| 231 | 1-[4-methoxy-2-(trifluoromethoxy)phenyl]-N-(1-phenylethyl)imidazo[1,5- |
| | d][1,2,4]triazin-4-amine |
| 232 | 1-[4-methoxy-2-(trifluoromethoxy)phenyl]-N-[1-(pyridin-3-yl)ethyl]imidazo[1,5- |
| | d][1,2,4]triazin-4-amine |
| 233 | 3-[{{1-[4-methoxy-2-(trifluoromethoxy)phenyl]imidazo[1,5-d][1,2,4]triazin-4- |
| | yl}amino)methyl]phenol |
| 234 | 1-[4-methoxy-2-(trifluoromethoxy)phenyl]-N-[(1H-pyrazol-3-yl)methyl]imidazo[1,5- |
| | d][1,2,4]triazin-4-amine |
| 235 | 3-({1-[4-methoxy-2-(trifluoromethyl)phenyl]imidazo[1,5-d][1,2,4]triazin-4- |
| | yl}amino)pyrrolidin-2-one |
| 236 | (1s,3s)-3-({1-[4-methoxy-2-(2,2,2-trifluoroethyl)phenyl]imidazo[1,5-d][1,2,4]triazin- |
| | 4-yl}amino)-1-methylcyclobutan-1-ol |
| 237 | (1R,2R)-2-({1-[4-methoxy-2-(2,2,2-trifluoroethyl)phenyl]imidazo[1,5- |
| | d][1,2,4]triazin-4-yl}amino)cyclohexan-1-ol |
| 238 | 3-({1-[4-methoxy-2-(2,2,2-trifluoroethyl)phenyl]imidazo[1,5-d][1,2,4]triazin-4- |
| | yl}amino)pyrrolidin-2-one |
| 239 | 2-[(3R)-3-({1-[2-(trifluoromethoxy)-4-(trifluoromethyl)phenyl]imidazo[1,5- |
| | d][1,2,4]triazin-4-yl}amino)piperidin-1-yl]ethan-1-ol |
| 240 | (1S,3R)-3-({1-[2-(trifluoromethoxy)-4-(trifluoromethyl)phenyl]imidazo[1,5- |
| | d][1,2,4]triazin-4-yl}amino)cyclohexan-1-ol |
| 241 | (1s,3s)-1-methyl-3-({1-[2-(trifluoromethoxy)-4-(trifluoromethyl)phenyl]imidazo[1,5- |
| | d][1,2,4]triazin-4-yl}amino)cyclobutan-1-ol |
| 242 | (1R,2R)-2-({1-[2-(trifluoromethoxy)-4-(trifluoromethyl)phenyl]imidazo[1,5- |
| | d][1,2,4]triazin-4-yl}amino)cyclohexan-1-ol |
| 243 | (1R,2R)-2-({1-[2,4-bis(trifluoromethyl)phenyl]imidazo[1,5-d][1,2,4]triazin-4- |
| | yl}amino)cyclohexan-1-ol |
| 244 | (1s,3s)-3-({1-[4-(difluoromethoxy)-2-(trifluoromethoxy)phenyl]imidazo[1,5- |
| | d][1,2,4]triazin-4-yl}amino)-1-methylcyclobutan-1-ol |
| 245 | (1S,3R)-3-({1-[4-(difluoromethoxy)-2-(trifluoromethoxy)phenyl]imidazo[1,5- |
| | d][1,2,4]triazin-4-yl}amino)cyclohexan-1-ol |
| 246 | (1R,2R)-2-({1-[4-(difluoromethoxy)-2-(trifluoromethoxy)phenyl]imidazo[1,5- |
| | d][1,2,4]triazin-4-yl}amino)cyclohexan-1-ol |
| 247 | 3-[(1S)-1-({1-[4-methoxy-2-(trifluoromethoxy)phenyl]imidazo[1,5-d][1,2,4]triazin-4- |
| | yl}amino)ethyl]phenol OR 3-[(1R)-1-({1-[4-methoxy-2- |
| | (trifluoromethoxy)phenyl]imidazo[1,5-d][1,2,4]triazin-4-yl}amino)ethyl]phenol |
| 248 | 3-[(1R)-1-({1-[4-methoxy-2-(trifluoromethoxy)phenyl]imidazo[1,5-d][1,2,4]triazin-4- |
| | yl}amino)ethyl]phenol OR 3-[(1S)-1-({1-[4-methoxy-2- |
| | (trifluoromethoxy)phenyl]imidazo[1,5-d][1,2,4]triazin-4-yl}amino)ethyl]phenol |
| 249 | (3S,4R)-4-({1-[4-methoxy-2-(2,2,2-trifluoroethyl)phenyl]imidazo[1,5- |
| | d][1,2,4]triazin-4-yl}amino)oxan-3-ol |
| 250 | 1-[4-methoxy-2-(2,2,2-trifluoroethyl)phenyl]-N-[(3R)-1-methylpiperidin-3- |
| | yl]imidazo[1,5-d][1,2,4]triazin-4-amine |
| 251 | 3-[1-({1-[4-methoxy-2-(trifluoromethyl)phenyl]imidazo[1,5-d][1,2,4]triazin-4- |
| | yl}amino)ethyl]phenol |
| 252 | 1-[2,4-bis(trifluoromethyl)phenyl]-N-[(3R,5R)-5-fluoro-1-methylpiperidin-3- |
| | yl]imidazo[1,5-d][1,2,4]triazin-4-amine |
| 253 | 2-[(3R)-3-({1-[2,4-bis(trifluoromethyl)phenyl]imidazo[1,5-d][1,2,4]triazin-4- |
| | yl}amino)piperidin-1-yl]ethan-1-ol |
| 254 | 1-[2,4-bis(trifluoromethyl)phenyl]-N-[(3R)-oxan-3-yl]imidazo[1,5-d][1,2,4]triazin-4- |
| | amine |
| 255 | [(1s,3s)-3-({1-[2-(trifluoromethoxy)-4-(trifluoromethyl)phenyl]imidazo[1,5- |
| | d][1,2,4]triazin-4-yl}amino)cyclobutyl]methanol |
| 256 | 3-({1-[2-(trifluoromethoxy)-4-(trifluoromethyl)phenyl]imidazo[1,5-d][1,2,4]triazin-4- |
| | yl}amino)pyrrolidin-2-one |
| 257 | [(1s,3s)-3-({1-[2,4-bis(trifluoromethyl)phenyl]imidazo[1,5-d][1,2,4]triazin-4- |
| | yl}amino)cyclobutyl]methanol |
| 258 | 2-[(3R)-3-({1-[4-(difluoromethoxy)-2-(trifluoromethoxy)phenyl]imidazo[1,5- |
| | d][1,2,4]triazin-4-yl}amino)piperidin-1-yl]ethan-1-ol |
| 259 | 3-[1-({1-[4-(difluoromethoxy)-2-(trifluoromethoxy)phenyl]imidazo[1,5- |
| | d][1,2,4]triazin-4-y]}amino)ethyl]phenol |
| 260 | 3-({1-[4-(difluoromethoxy)-2-(trifluoromethoxy)phenyl]imidazo[1,5-d][1,2,4]triazin- |
| | 4-yl}amino)pyrrolidin-2-one |
| 261 | N-[(3R)-1-ethylpiperidin-3-yl]-1-[2-(2,2,2-trifluoroethyl)-4- |
| | (trifluoromethyl)phenyl]imidazo[1,5-d][1,2,4]triazin-4-amine |
| 262 | (1S,3R)-3-({1-[2-(2,2,2-trifluoroethyl)-4-(trifluoromethyl)phenyl]imidazo[1,5- |
| | d][1,2,4]triazin-4-yl}amino)cyclohexan-1-ol |
| 263 | (1s,3s)-1-methyl-3-({1-[2-(2,2,2-trifluoroethyl)-4- |
| | (trifluoromethyl)phenyl]imidazo[1,5-d][1,2,4]triazin-4-yl}amino)cyclobutan-1-ol |
| 264 | [(1s,3s)-3-({1-[2-(2,2,2-trifluoroethyl)-4-(trifluoromethyl)phenyl]imidazo[1,5- |
| | d][1,2,4]triazin-4-y]}amino)cyclobutyl]methanol |
| 265 | 3-({1-[2-(2,2,2-trifluoroethyl)-4-(trifluoromethyl)phenyl]imidazo[1,5-d][1,2,4]triazin- |
| | 4-yl}amino)pyrrolidin-2-one |
| 266 | 3-[1-({1-[2-(2,2,2-trifluoroethyl)-4-(trifluoromethyl)phenyl]imidazo[1,5- |
| | d][1,2,4]triazin-4-y]}amino)ethyl]phenol |
| 267 | N-(1H-pyrazol-3-yl)-1-[2-(2,2,2-trifluoroethyl)-4- |
| | (trifluoromethyl)phenyl]imidazo[1,5-d][1,2,4]triazin-4-amine |
| 268 | (3S,4R)-4-({1-[2-(difluoromethoxy)-4-methoxyphenyl]imidazo[1,5-d][1,2,4]triazin-4- |
| | yl}amino)oxan-3-ol |
| 269 | 3-({1-[2-(difluoromethoxy)-4-methoxyphenyl]imidazo[1,5-d][1,2,4]triazin-4- |
| | yl}amino)pyrrolidin-2-one |
| 270 | (1R,2R)-2-({1-[2-(difluoromethoxy)-4-methoxyphenyl]imidazo[1,5-d][1,2,4]triazin- |
| | 4-yl}amino)cyclohexan-1-ol |
| 271 | 1-[2,4-bis(trifluoromethyl)phenyl]-N-[(3R)-1-ethylpiperidin-3-yl]imidazo[1,5- |
| | d][1,2,4]triazin-4-amine |
| 272 | 3-({1-[2,4-bis(trifluoromethyl)phenyl]imidazo[1,5-d][1,2,4]triazin-4-yl}amino)-1- |
| | methylpiperidin-2-one |
| 273 | (3S,4R)-4-({1-[2,4-bis(trifluoromethyl)phenyl]imidazo[1,5-d][1,2,4]triazin-4- |
| | yl}amino)oxan-3-ol |
| 274 | (3S,4S)-3-({1-[2,4-bis(trifluoromethyl)phenyl]imidazo[1,5-d][1,2,4]triazin-4- |
| | yl}amino)oxan-4-ol |
| 275 | (1s,4s)-4-({1-[2,4-bis(trifluoromethyl)phenyl]imidazo[1,5-d][1,2,4]triazin-4- |
| | yl}amino)cyclohexan-1-ol |
| 276 | 3-({1-[2,4-bis(trifluoromethyl)phenyl]imidazo[1,5-d][1,2,4]triazin-4- |
| | yl}amino)piperidin-2-one |
| 277 | N-[(3R,5R)-5-fluoro-1-methylpiperidin-3-yl]-1-[4-methoxy-2-(2,2,2- |
| | trifluoroethyl)phenyl]imidazo[1,5-d][1,2,4]triazin-4-amine |
| 278 | 2-[(3R)-3-({1-[4-methoxy-2-(2,2,2-trifluoroethyl)phenyl]imidazo[1,5-d][1,2,4]triazin- |
| | 4-yl}amino)piperidin-1-yl]ethan-1-ol |
| 279 | 8-methyl-N-[(3R)-1-methylpiperidin-3-yl]-1-[2-(2,2,2-trifluoroethyl)-4- |
| | (trifluoromethyl)phenyl]imidazo[1,5-d][1,2,4]triazin-4-amine |
| 280 | (1R,2R)-2-({8-methyl-1-[2-(2,2,2-trifluoroethyl)-4- |
| | (trifluoromethyl)phenyl]imidazo[1,5-d][1,2,4]triazin-4-yl}amino)cyclohexan-1-ol |
| 281 | 1-[4-methoxy-2-(pentafluoroethyl)phenyl]-N-[(3R)-1-methylpiperidin-3- |
| | yl]imidazo[1,5-d][1,2,4]triazin-4-amine |
| 282 | (1R,2R)-2-({1-[2-(2,2,2-trifluoroethyl)-4-(trifluoromethyl)phenyl]imidazo[1,5- |
| | d][1,2,4]triazin-4-yl}amino)cyclohexan-1-ol |
| 283 | (1S,2S)-1-methyl-2-({1-[2-(2,2,2-trifluoroethyl)-4- |
| | (trifluoromethyl)phenyl]imidazo[1,5-d][1,2,4]triazin-4-yl}amino)cyclohexan-1-ol |
| | AND (1R,2R)-1-methyl-2-({1-[2-(2,2,2-trifluoroethyl)-4- |
| | (trifluoromethyl)phenyl]imidazo[1,5-d][1,2,4]triazin-4-yl}amino)cyclohexan-1-ol |
| 284 | 3-({1-[4-methoxy-2-(trifluoromethoxy)phenyl]imidazo[1,5-d][1,2,4]triazin-4- |
| | yl}amino)pyrrolidin-2-one |
| 285 | 1-[2,4-bis(trifluoromethyl)phenyl]-N-(1H-pyrazol-3-yl)imidazo[1,5-d][1,2,4]triazin- |
| | 4-amine |
| 286 | (1R,2R)-2-({1-[4-methyl-2-(2,2,2-trifluoroethyl)phenyl]imidazo[1,5-d][1,2,4]triazin- |
| | 4-yl}amino)cyclohexan-1-ol |
| 287 | 3-({1-[4-chloro-2-(trifluoromethoxy)phenyl]imidazo[1,5-d][1,2,4]triazin-4- |
| | yl}amino)pyrrolidin-2-one |
| 288 | 3-({1-[4-methyl-2-(2,2,2-trifluoroethyl)phenyl]imidazo[1,5-d][1,2,4]triazin-4- |
| | yl}amino)pyrrolidin-2-one |
| 289 | (3S,4R)-1-methyl-3-({1-[2-(2,2,2-trifluoroethyl)-4- |
| | (trifluoromethyl)phenyl]imidazo[1,5-d][1,2,4]triazin-4-yl}amino)piperidin-4-ol |
| 290 | (1S,2S)-2-({1-[2-(trifluoromethoxy)-4-(trifluoromethyl)phenyl]imidazo[1,5- |
| | d][1,2,4]triazin-4-y]}amino)cyclohexan-1-ol |
| 291 | [(1s,3s)-3-({1-[4-methoxy-2-(trifluoromethoxy)phenyl]imidazo[1,5-d][1,2,4]triazin-4- |
| | yl}amino)cyclobutyl]methanol |
| 292 | (3S,4R)-4-({1-[4-methoxy-2-(trifluoromethoxy)phenyl]imidazo[1,5-d][1,2,4]triazin- |
| | 4-yl}amino)oxan-3-ol |
| 293 | (1S,2S)-2-({1-[2,4-bis(trifluoromethyl)phenyl]imidazo[1,5-d][1,2,4]triazin-4- |
| | yl}amino)cyclohexan-1-ol |
| 294 | 3-({1-[2,4-bis(trifluoromethyl)phenyl]imidazo[1,5-d][1,2,4]triazin-4- |
| | yl}amino)pyrrolidin-2-one |
| 295 | 1-[4-(difluoromethoxy)-2-(trifluoromethoxy)phenyl]-N-[(3R)-1-methylpiperidin-3- |
| | yl]imidazo[1,5-d][1,2,4]triazin-4-amine |
| 296 | 1-[4-(difluoromethoxy)-2-(trifluoromethoxy)phenyl]-N-(1H-pyrazol-3- |
| | yl)imidazo[1,5-d][1,2,4]triazin-4-amine |
| 297 | 3-[1-({1-[4-methyl-2-(2,2,2-trifluoroethyl)phenyl]imidazo[1,5-d][1,2,4]triazin-4- |
| | yl}amino)ethyl]phenol |
| 298 | (1s,3s)-3-({1-[4-chloro-2-(trifluoromethyl)phenyl]imidazo[1,5-d][1,2,4]triazin-4- |
| | yl}amino)-1-methylcyclobutan-1-ol |
| 299 | (1s,3s)-3-({1-[4-chloro-2-(trifluoromethoxy)phenyl]imidazo[1,5-d][1,2,4]triazin-4- |
| | yl}amino)-1-methylcyclobutan-1-ol |
| 300 | (1R,2R)-2-({1-[4-chloro-2-(trifluoromethoxy)phenyl]imidazo[1,5-d][1,2,4]triazin-4- |
| | yl}amino)cyclohexan-1-ol |
| 301 | 2-[(3R)-3-({1-[2-(2,2,2-trifluoroethyl)-4-(trifluoromethyl)phenyl]imidazo[1,5- |
| | d][1,2,4]triazin-4-y]}amino)piperidin-1-yl]ethan-1-ol |
| 302 | (1S,3R)-3-({1-[4-methoxy-2-(2,2,2-trifluoroethyl)phenyl]imidazo[1,5- |
| | d][1,2,4]triazin-4-yl}amino)cyclohexan-1-ol |
| 303 | 3-[1-({1-[4-methoxy-2-(2,2,2-trifluoroethyl)phenyl]imidazo[1,5-d][1,2,4]triazin-4- |
| | yl}amino)ethyl]phenol |
| 304 | 1-[4-methoxy-2-(2,2,2-trifluoroethoxy)phenyl]-N-[(3R)-1-methylpiperidin-3- |
| | yl]imidazo[1,5-d][1,2,4]triazin-4-amine |
| 305 | (1s,3s)-3-({1-[4-methoxy-2-(2,2,2-trifluoroethoxy)phenyl]imidazo[1,5- |
| | d][1,2,4]triazin-4-yl}amino)-1-methylcyclobutan-1-ol |
| 306 | (1R,2R)-2-({1-[4-bromo-2-(difluoromethoxy)phenyl]imidazo[1,5-d][1,2,4]triazin-4- |
| | yl}amino)cyclohexan-1-ol |
| 307 | (1R,2R)-2-({1-[4-bromo-2-(trifluoromethoxy)phenyl]imidazo[1,5-d][1,2,4]triazin-4- |
| | yl}amino)cyclohexan-1-ol |
| 308 | 1-[4-methoxy-2-(trifluoromethoxy)phenyl]-N-[(1S)-1-(pyridin-3- |
| | yl)ethyl]imidazo[1,5-d][1,2,4]triazin-4-amine OR 1-[4-methoxy-2- |
| | (trifluoromethoxy)phenyl]-N-[(1R)-1-(pyridin-3-yl)ethyl]imidazo[1,5-d][1,2,4]triazin- |
| | 4-amine |
| 309 | 1-[4-methoxy-2-(trifluoromethoxy)phenyl]-N-[(1R)-1-(pyridin-3- |
| | yl)ethyl]imidazo[1,5-d][1,2,4]triazin-4-amine OR 1-[4-methoxy-2- |
| | (trifluoromethoxy)phenyl]-N-[(1S)-1-(pyridin-3-yl)ethyl]imidazo[1,5-d][1,2,4]triazin- |
| | 4-amine |
| 310 | (1S,2S)-2-({1-[4-methoxy-2-(trifluoromethoxy)phenyl]imidazo[1,5-d][1,2,4]triazin-4- |
| | yl}amino)-1-methylcyclohexan-1-ol OR (1R,2R)-2-({1-[4-methoxy-2- |
| | (trifluoromethoxy)phenyl]imidazo[1,5-d][1,2,4]triazin-4-yl}amino)-1- |
| | methylcyclohexan-1-ol |
| 311 | (1R,2R)-2-({1-[4-methoxy-2-(trifluoromethoxy)phenyl]imidazo[1,5-d][1,2,4]triazin- |
| | 4-yl}amino)-1-methylcyclohexan-1-ol OR (1S,2S)-2-({1-[4-methoxy-2- |
| | (trifluoromethoxy)phenyl]imidazo[1,5-d][1,2,4]triazin-4-yl}amino)-1- |
| | methylcyclohexan-1-ol |
| 312 | 1-[4-bromo-2-(trifluoromethoxy)phenyl]-N-[(3R)-1-methylpiperidin-3- |
| | yl]imidazo[1,5-d][1,2,4]triazin-4-amine |
| 313 | (3S,4R)-4-({1-[2-(2,2,2-trifluoroethyl)-4-(trifluoromethyl)phenyl]imidazo[1,5- |
| | d][1,2,4]triazin-4-yl}amino)oxan-3-ol |
| 314 | [(1s,3s)-3-({1-[4-methoxy-2-(2,2,2-trifluoroethyl)phenyl]imidazo[1,5-d][1,2,4]triazin- |
| | 4-yl}amino)cyclobutyl]methanol |
| 315 | (1s,3s)-3-({1-[4-methoxy-2-(pentafluoroethyl)phenyl]imidazo[1,5-d][1,2,4]triazin-4- |
| | yl}amino)-1-methylcyclobutan-1-ol |
| 316 | (1R,2R)-2-({1-[4-methoxy-2-(pentafluoroethyl)phenyl]imidazo[1,5-d][1,2,4]triazin-4- |
| | yl}amino)cyclohexan-1-ol |
| 317 | N-[(3R)-1-methylpiperidin-3-yl]-1-[4-methyl-2-(2,2,2- |
| | trifluoroethyl)phenyl]imidazo[1,5-d][1,2,4]triazin-4-amine |
| 318 | (1s,3s)-1-methyl-3-({1-[4-methyl-2-(2,2,2-trifluoroethyl)phenyl]imidazo[1,5- |
| | d][1,2,4]triazin-4-yl}amino)cyclobutan-1-ol |
| 319 | (1s,3s)-3-{[1-(2-cyclopropyl-4-methoxyphenyl)imidazo[1,5-d][1,2,4]triazin-4- |
| | yl]amino}-1-methylcyclobutan-1-ol |
| 320 | 3-{[1-(2-cyclopropyl-4-methoxyphenyl)imidazo[1,5-d][1,2,4]triazin-4- |
| | yl]amino}pyrrolidin-2-one |
| 321 | (1R,2R)-2-{[1-(2-cyclopropyl-4-methoxyphenyl)imidazo[1,5-d][1,2,4]triazin-4- |
| | yl]amino}cyclohexan-1-ol |
| 322 | (3S,4R)-4-({1-[2-(trifluoromethoxy)-4-(trifluoromethyl)phenyl]imidazo[1,5- |
| | d][1,2,4]triazin-4-yl}amino)oxan-3-ol |
| 323 | 3-[1-({1-[2-(trifluoromethoxy)-4-(trifluoromethyl)phenyl]imidazo[1,5- |
| | d][1,2,4]triazin-4-yl}amino)ethyl]phenol |
| 324 | N-[3-(methoxymethyl)cyclobutyl]-1-[2-(trifluoromethoxy)-4- |
| | (trifluoromethyl)phenyl]imidazo[1,5-d][1,2,4]triazin-4-amine |
| 325 | 2-[3-({1-[2-(trifluoromethoxy)-4-(trifluoromethyl)phenyl]imidazo[1,5- |
| | d][1,2,4]triazin-4-yl}amino)cyclobutyl]propan-2-ol |
| 326 | N-[3-(methoxymethyl)cyclobutyl]-1-[4-methoxy-2-(2,2,2- |
| | trifluoroethyl)phenyl]imidazo[1,5-d][1,2,4]triazin-4-amine |
| 327 | 2-[3-({1-[4-methoxy-2-(2,2,2-trifluoroethyl)phenyl]imidazo[1,5-d][1,2,4]triazin-4- |
| | yl}amino)cyclobutyl]propan-2-ol |
| 328 | 3-({1-[4-(difluoromethoxy)-2-(trifluoromethoxy)phenyl]imidazo[1,5-d][1,2,4]triazin- |
| | 4-yl}amino)-1-methylpiperidin-2-one |
| 329 | 1-methyl-3-({1-[2-(2,2,2-trifluoroethyl)-4-(trifluoromethyl)phenyl]imidazo[1,5- |
| | d][1,2,4]triazin-4-yl}amino)piperidin-2-one |
| 330 | 3-({1-[4-methoxy-2-(2,2,2-trifluoroethyl)phenyl]imidazo[1,5-d][1,2,4]triazin-4- |
| | yl}amino)-1-methylpiperidin-2-one |
| 331 | (3S,4R)-4-({1-[4-(difluoromethoxy)-2-(trifluoromethoxy)phenyl]imidazo[1,5- |
| | d][1,2,4]triazin-4-yl}amino)oxan-3-ol |
| 332 | (1s,3s)-1-methyl-3-({1-[4-methyl-2-(trifluoromethoxy)phenyl]imidazo[1,5- |
| | d][1,2,4]triazin-4-yl}amino)cyclobutan-1-ol |
| 333 | 3-({1-[2-cyclopropyl-4-(trifluoromethyl)phenyl]imidazo[1,5-d][1,2,4]triazin-4- |
| | yl}amino)pyrrolidin-2-one |
| 334 | (3S,4R)-4-({1-[2-cyclopropyl-4-(trifluoromethyl)phenyl]imidazo[1,5-d][1,2,4]triazin- |
| | 4-yl}amino)oxan-3-ol |
| 335 | [1-methyl-3-({1-[2-(trifluoromethoxy)-4-(trifluoromethyl)phenyl]imidazo[1,5- |
| | d][1,2,4]triazin-4-yl}amino)cyclobutyl]methanol |
| 336 | [(1r,3r)-3-({1-[4-methoxy-2-(2,2,2-trifluoroethyl)phenyl]imidazo[1,5-d][1,2,4]triazin- |
| | 4-yl}amino)-1-methylcyclobutyl]methanol |
| 337 | [3-({1-[4-methoxy-2-(2,2,2-trifluoroethyl)phenyl]imidazo[1,5-d][1,2,4]triazin-4- |
| | yl}amino)-1-methylcyclobutyl]methanol |
| 338 | 2-[3-({1-[4-methoxy-2-(trifluoromethoxy)phenyl]imidazo[1,5-d][1,2,4]triazin-4- |
| | yl}amino)cyclobutyl]propan-2-ol |
| 339 | 2-[3-({1-[2-(2,2,2-trifluoroethyl)-4-(trifluoromethyl)phenyl]imidazo[1,5- |
| | d][1,2,4]triazin-4-yl}amino)cyclobutyl]propan-2-ol |
| 340 | [(1s,3s)-1-methyl-3-({1-[2-(trifluoromethoxy)-4-(trifluoromethyl)phenyl]imidazo[1,5- |
| | d][1,2,4]triazin-4-yl}amino)cyclobutyl]methanol |
| 341 | [(1s,3s)-3-({1-[4-methoxy-2-(2,2,2-trifluoroethyl)phenyl]imidazo[1,5-d][1,2,4]triazin- |
| | 4-yl}amino)-1-methylcyclobutyl]methanol |
| 342 | [(1s,3s)-3-({1-[4-methoxy-2-(trifluoromethoxy)phenyl]imidazo[1,5-d][1,2,4]triazin-4- |
| | yl}amino)-1-methylcyclobutyl]methanol |
| 343 | [(1s,3s)-1-methyl-3-({1-[2-(2,2,2-trifluoroethyl)-4- |
| | (trifluoromethyl)phenyl]imidazo[1,5-d][1,2,4]triazin-4-yl}amino)cyclobutyl]methanol |
| 344 | (1s,3s)-3-({1-[2-(difluoromethoxy)-4-methylphenyl]imidazo[1,5-d][1,2,4]triazin-4- |
| | yl}amino)-1-methylcyclobutan-1-ol |
| 345 | (1s,3s)-3-({1-[4-cyclopropyl-2-(difluoromethoxy)phenyl]imidazo[1,5-d][1,2,4]triazin- |
| | 4-yl}amino)-1-methylcyclobutan-1-ol |
| 346 | (1s,3s)-3-({1-[4-cyclopropyl-2-(trifluoromethoxy)phenyl]imidazo[1,5- |
| | d][1,2,4]triazin-4-yl}amino)-1-methylcyclobutan-1-ol |
| 347 | [(1s,3s)-3-({1-[4-methyl-2-(trifluoromethoxy)phenyl]imidazo[1,5-d][1,2,4]triazin-4- |
| | yl}amino)cyclobutyl]methanol |
| 348 | (1s,3s)-3-({1-[4-cyclopropyl-2-(2,2,2-trifluoroethyl)phenyl]imidazo[1,5- |
| | d][1,2,4]triazin-4-yl}amino)-1-methylcyclobutan-1-ol |
| 349 | [(1s,3s)-3-({1-[4-cyclopropyl-2-(2,2,2-trifluoroethyl)phenyl]imidazo[1,5- |
| | d][1,2,4]triazin-4-yl}amino)cyclobutyl]methanol |
| 350 | 3-({1-[4-cyclopropyl-2-(2,2,2-trifluoroethyl)phenyl]imidazo[1,5-d][1,2,4]triazin-4- |
| | yl}amino)pyrrolidin-2-one |
| 351 | (3S,4R)-4-({1-[4-cyclopropyl-2-(2,2,2-trifluoroethyl)phenyl]imidazo[1,5- |
| | d][1,2,4]triazin-4-yl}amino)oxan-3-ol |
| 352 | 1-[4-cyclopropyl-2-(2,2,2-trifluoroethyl)phenyl]-N-[(3R)-1-methylpiperidin-3- |
| | yl]imidazo[1,5-d][1,2,4]triazin-4-amine |
| 353 | (1S,3R)-3-({1-[4-cyclopropyl-2-(2,2,2-trifluoroethyl)phenyl]imidazo[1,5- |
| | d][1,2,4]triazin-4-yl}amino)cyclohexan-1-ol |
| 354 | 3-[1-({1-[4-cyclopropyl-2-(2,2,2-trifluoroethyl)phenyl]imidazo[1,5-d][1,2,4]triazin-4- |
| | yl}amino)ethyl]phenol |
| 355 | 2-[(3R)-3-({1-[4-methyl-2-(trifluoromethoxy)phenyl]imidazo[1,5-d][1,2,4]triazin-4- |
| | yl}amino)piperidin-1-yl]ethan-1-ol |
| 356 | 4-[2-(difluoromethoxy)-4-(trifluoromethyl)phenyl]-N-[(3R,5R)-1-ethyl-5- |
| | fluoropiperidin-3-yl]-2-methylpyrazolo[1,5-d][1,2,4]triazin-7-amine |
| 357 | 4-[4-bromo-2-(trifluoromethoxy)phenyl]-N-[(3R)-1-ethylpiperidin-3-yl]-2- |
| | methylpyrazolo[1,5-d][1,2,4]triazin-7-amine |
| 358 | 4-[4-chloro-2-(difluoromethoxy)phenyl]-2-methyl-N-[(3R)-1-methylpiperidin-3- |
| | yl]pyrazolo[1,5-d][1,2,4]triazin-7-amine |
| 359 | 4-[2-(difluoromethyl)-4-(trifluoromethyl)phenyl]-2-methyl-N-[(3R)-1- |
| | methylpiperidin-3-yl]pyrazolo[1,5-d][1,2,4]triazin-7-amine |
| 360 | 4-[4-cyclopropyl-2-(difluoromethoxy)phenyl]-2-methyl-N-[(3R)-1-methylpiperidin-3- |
| | yl]pyrazolo[1,5-d][1,2,4]triazin-7-amine |
| 361 | 4-[2-(difluoromethoxy)-4-methylphenyl]-2-methyl-N-[(3R)-1-methylpiperidin-3- |
| | yl]pyrazolo[1,5-d][1,2,4]triazin-7-amine |
| 362 | 4-[4-chloro-2-(difluoromethyl)phenyl]-2-methyl-N-[(3R)-1-methylpiperidin-3- |
| | yl]pyrazolo[1,5-d][1,2,4]triazin-7-amine |
| 363 | 4-[4-cyclopropyl-2-(difluoromethyl)phenyl]-2-methyl-N-[(3R)-1-methylpiperidin-3- |
| | yl]pyrazolo[1,5-d][1,2,4]triazin-7-amine |
| 364 | 4-[2-(difluoromethyl)-4-methylphenyl]-2-methyl-N-[(3R)-1-methylpiperidin-3- |
| | yl]pyrazolo[1,5-d][1,2,4]triazin-7-amine |
| 365 | N-[(3R)-1-ethylpiperidin-3-yl]-2-methyl-4-[2-(trifluoromethoxy)-4- |
| | (trifluoromethyl)phenyl]pyrazolo[1,5-d][1,2,4]triazin-7-amine |
| 366 | N-[(3R)-1-ethylpiperidin-3-yl]-2-methyl-4-[4-(methylsulfanyl)-2- |
| | (trifluoromethoxy)phenyl]pyrazolo[1,5-d][1,2,4]triazin-7-amine |
| 367 | N-[(3R)-1-ethylpiperidin-3-yl]-2-methyl-4-[4-methyl-2- |
| | (methylsulfanyl)phenyl]pyrazolo[1,5-d][1,2,4]triazin-7-amine |
| 368 | N-[(3R)-1-ethylpiperidin-3-yl]-4-[2-(methanesulfonyl)-4-methylphenyl]-2- |
| | methylpyrazolo[1,5-d][1,2,4]triazin-7-amine |
| 369 | N-[(3R)-1-ethylpiperidin-3-yl]-4-[4-(methanesulfonyl)-2-(trifluoromethoxy)phenyl]- |
| | 2-methylpyrazolo[1,5-d][1,2,4]triazin-7-amine |
| 370 | N-[(3R)-1-ethylpiperidin-3-yl]-2-methyl-4-(2,3,4-trimethoxyphenyl)pyrazolo[1,5- |
| | d][1,2,4]triazin-7-amine |
| 371 | N-[(3R)-1-ethylpiperidin-3-yl]-4-[4-methoxy-2-(trifluoromethoxy)phenyl]-2- |
| | methylpyrazolo[1,5-d][1,2,4]triazin-7-amine |
| 372 | 2-(7-{[(3R)-1-ethylpiperidin-3-yl]amino}-2-methylpyrazolo[1,5-d][1,2,4]triazin-4- |
| | yl)-5-(trifluoromethyl)benzamide |
| 373 | N-[(3R)-1-ethylpiperidin-3-yl]-4-[4-methoxy-2-(trifluoromethyl)phenyl]-2- |
| | methylpyrazolo[1,5-d][1,2,4]triazin-7-amine |
| 374 | 4-(2-chloro-4-methoxyphenyl)-N-[(3R)-1-ethylpiperidin-3-yl]-2-methylpyrazolo[1,5- |
| | d][1,2,4]triazin-7-amine |
| 375 | 4-[2-chloro-4-(trifluoromethyl)phenyl]-N-[(3R)-1-ethylpiperidin-3-yl]-2- |
| | methylpyrazolo[1,5-d][1,2,4]triazin-7-amine |
| 376 | 4-(2,4-dichlorophenyl)-N-[(3R)-1-ethylpiperidin-3-yl]-2-methylpyrazolo[1,5- |
| | d][1,2,4]triazin-7-amine |
| 377 | 4-(4-chlorophenyl)-N-[(3R)-1-ethylpiperidin-3-yl]-2-methylpyrazolo[1,5- |
| | d][1,2,4]triazin-7-amine |
| 378 | 3-(difluoromethoxy)-4-(7-{[(3R)-1-ethylpiperidin-3-yl]amino}-2-methylpyrazolo[1,5- |
| | d][1,2,4]triazin-4-yl)benzonitrile |
| 379 | 4-(2-cyclopropyl-4-methoxyphenyl)-N-[(3R)-1-ethylpiperidin-3-yl]-2- |
| | methylpyrazolo[1,5-d][1,2,4]triazin-7-amine |
| 380 | (1s,3s)-3-({4-[2-(difluoromethoxy)-4-(trifluoromethyl)phenyl]-2-methylpyrazolo[1,5- |
| | d][1,2,4]triazin-7-yl}amino)-1-methylcyclobutan-1-ol |
| 381 | (1R,2R)-2-({4-[2-(difluoromethoxy)-4-(trifluoromethyl)phenyl]-2- |
| | methylpyrazolo[1,5-d][1,2,4]triazin-7-yl}amino)cyclohexan-1-ol |
| 382 | (1R,2R)-2-({2-methyl-4-[2-(trifluoromethoxy)-4- |
| | (trifluoromethyl)phenyl]pyrazolo[1,5-d][1,2,4]triazin-7-yl}amino)cyclohexan-1-ol |
| 383 | 3-[1-({4-[2-(difluoromethoxy)-4-(trifluoromethyl)phenyl]-2-methylpyrazolo[1,5- |
| | d][1,2,4]triazin-7-yl}amino)ethyl]phenol |
| 384 | (3S,4R)-4-({4-[4-methoxy-2-(2,2,2-trifluoroethyl)phenyl]-2-methylpyrazolo[1,5- |
| | d][1,2,4]triazin-7-yl}amino)oxan-3-ol |
| 385 | (1R,2R)-2-({4-[4-methoxy-2-(2,2,2-trifluoroethyl)phenyl]-2-methylpyrazolo[1,5- |
| | d][1,2,4]triazin-7-yl}amino)cyclohexan-1-ol |
| 386 | 3-({4-[4-methoxy-2-(2,2,2-trifluoroethyl)phenyl]-2-methylpyrazolo[1,5- |
| | d][1,2,4]triazin-7-yl}amino)pyrrolidin-2-one |
| 387 | (1s,3s)-3-({4-[4-methoxy-2-(2,2,2-trifluoroethyl)phenyl]-2-methylpyrazolo[1,5- |
| | d][1,2,4]triazin-7-yl}amino)-1-methylcyclobutan-1-ol |
| 388 | (1s,3s)-1-methyl-3-({2-methyl-4-[2-(2,2,2-trifluoroethyl)-4- |
| | (trifluoromethyl)phenyl]pyrazolo[1,5-d][1,2,4]triazin-7-yl}amino)cyclobutan-1-ol |
| 389 | (1R,2R)-2-({2-methyl-4-[2-(2,2,2-trifluoroethyl)-4- |
| | (trifluoromethyl)phenyl]pyrazolo[1,5-d][1,2,4]triazin-7-yl}amino)cyclohexan-1-ol |
| 390 | (1s,3s)-1-methyl-3-({2-methyl-4-[4-methyl-2-(2,2,2- |
| | trifluoroethyl)phenyl]pyrazolo[1,5-d][1,2,4]triazin-7-yl}amino)cyclobutan-1-ol |
| 391 | (1s,3s)-3-({4-[4-methoxy-2-(trifluoromethoxy)phenyl]-2-methylpyrazolo[1,5- |
| | d][1,2,4]triazin-7-yl}amino)-1-methylcyclobutan-1-ol |
| 392 | (1R,2R)-2-({4-[4-methoxy-2-(trifluoromethoxy)phenyl]-2-methylpyrazolo[1,5- |
| | d][1,2,4]triazin-7-yl}amino)cyclohexan-1-ol |
| 393 | 3-({4-[2-(trifluoromethoxy)-4-(trifluoromethyl)phenyl]pyrazolo[1,5-d][1,2,4]triazin- |
| | 7-yl}amino)pyrrolidin-2-one |
| 394 | (1R,2R)-2-({4-[2,4-bis(trifluoromethyl)phenyl]pyrazolo[1,5-d][1,2,4]triazin-7- |
| | yl}amino)cyclohexan-1-ol |
| 395 | 3-[1-({4-[2,4-bis(trifluoromethyl)phenyl]pyrazolo[1,5-d][1,2,4]triazin-7- |
| | yl}amino)ethyl]phenol |
| 396 | 3-({4-[2,4-bis(trifluoromethyl)phenyl]pyrazolo[1,5-d][1,2,4]triazin-7- |
| | yl}amino)pyrrolidin-2-one |
| 397 | (1s,3s)-1-methyl-3-({4-[2-(trifluoromethoxy)-4-(trifluoromethyl)phenyl]pyrazolo[1,5- |
| | d][1,2,4]triazin-7-yl}amino)cyclobutan-1-ol |
| 398 | (1R,2R)-2-({4-[2-(trifluoromethoxy)-4-(trifluoromethyl)phenyl]pyrazolo[1,5- |
| | d][1,2,4]triazin-7-yl}amino)cyclohexan-1-ol |
| 399 | (1s,3s)-3-({1-[2-cyclopropyl-4-(trifluoromethyl)phenyl]imidazo[1,5-d][1,2,4]triazin- |
| | 4-yl}amino)-1-methylcyclobutan-1-ol |
| 400 | (3S,4S)-3-({1-[2-(2,2,2-trifluoroethyl)-4-(trifluoromethyl)phenyl]imidazo[1,5- |
| | d][1,2,4]triazin-4-yl}amino)oxan-4-ol |
| 401 | (1S,2R)-2-({1-[2-(2,2,2-trifluoroethyl)-4-(trifluoromethyl)phenyl]imidazo[1,5- |
| | d][1,2,4]triazin-4-yl}amino)cyclohexan-1-ol |
| 402 | (3S,4R)-4-({1-[4-methyl-2-(2,2,2-trifluoroethyl)phenyl]imidazo[1,5-d][1,2,4]triazin- |
| | 4-yl}amino)oxan-3-ol |

wherein a form of the compound is selected from the group consisting of a pharmaceutically acceptable salt, hydrate, solvate, racemate, enantiomer, diastereomer, stereoisomer, tautomer, and isotope enriched form thereof.

In embodiment 3, the invention provides compounds selected from:

| Compound # | Name |
|---|---|
| 2 | N-[(3R)-1-methylpiperidin-3-yl]-1-[2-(2,2,2-trifluoroethyl)-4- |
| | (trifluoromethyl)phenyl]imidazo[1,5-d][1,2,4]triazin-4-amine formate |
| 12 | 1-[2-(difluoromethoxy)-4-methoxyphenyl]-7-methyl-N-[(3R)-1-methylpiperidin-3- |
| | yl]pyrrolo[1,2-d][1,2,4]triazin-4-amine formate |
| 13 | 1-[4-chloro-2-(difluoromethoxy)phenyl]-7-methyl-N-[(3R)-1-methylpiperidin-3- |
| | yl]pyrrolo[1,2-d][1,2,4]triazin-4-amine formate |
| 14 | 1-[4-cyclopropyl-2-(difluoromethyl)phenyl]-7-methyl-N-[(3R)-1-methylpiperidin-3- |
| | yl]pyrrolo[1,2-d][1,2,4]triazin-4-amine formate |
| 19 | 1-[2-fluoro-4-(trifluoromethyl)phenyl]-N-[(3R)-1-methylpiperidin-3-yl]pyrrolo[1,2- |
| | d][1,2,4]triazin-4-amine formate |
| 23 | 1-[2-(difluoromethyl)-4-methylphenyl]-7-methyl-N-[(3R)-1-methylpiperidin-3- |
| | yl]pyrrolo[1,2-d][1,2,4]triazin-4-amine formate |
| 24 | 1-[4-cyclopropyl-2-(difluoromethoxy)phenyl]-7-methyl-N-[(3R)-1-methylpiperidin-3- |
| | yl]pyrrolo[1,2-d][1,2,4]triazin-4-amine formate |
| 25 | 1-[2-(difluoromethoxy)-4-methylphenyl]-7-methyl-N-[(3R)-1-methylpiperidin-3- |
| | yl]pyrrolo[1,2-d][1,2,4]triazin-4-amine formate |
| 34 | 1-[2-(difluoromethoxy)-4-methylphenyl]-N-[(3R)-1-ethylpiperidin-3-yl]pyrrolo[1,2- |
| | d][1,2,4]triazin-4-amine formate |
| 35 | 1-[2-(difluoromethoxy)-4-(trifluoromethyl)phenyl]-N-[(3R)-piperidin-3-yl]pyrrolo[1,2- |
| | d][1,2,4]triazin-4-amine hydrochloride |
| 37 | 1-[4-cyclopropyl-2-(trifluoromethoxy)phenyl]-N-[(3R)-1-methylpiperidin-3- |
| | yl]pyrrolo[1,2-d][1,2,4]triazin-4-amine formate |
| 42 | 1-[4-ethyl-2-(trifluoromethoxy)phenyl]-N-[(3R)-1-methylpiperidin-3-yl]pyrrolo[1,2- |
| | d][1,2,4]triazin-4-amine formate |
| 74 | 1-[4-methoxy-2-(trifluoromethoxy)phenyl]-N-{[(2S)-1-methylpyrrolidin-2- |
| | yl]methyl}pyrrolo[1,2-d][1,2,4]triazin-4-amine trifluoroacetate |
| 95 | 1-[3-(difluoromethoxy)-4-(4-{[(3R)-1-methylpiperidin-3-yl]amino}pyrrolo[1,2- |
| | d][1,2,4]triazin-1-yl)phenyl]propan-1-one formate |
| 134 | (3S,4R)-3-({1-[2,4-bis(trifluoromethyl)phenyl]-7-methylpyrrolo[1,2-d][1,2,4]triazin-4- |
| | yl}amino)-1-ethylpiperidin-4-ol formate |
| 145 | 1-[4-methoxy-2-(2,2,2-trifluoroethyl)phenyl]-N-[(3R)-1-methylpiperidin-3- |
| | yl]pyrrolo[1,2-d][1,2,4]triazin-4-amine formate |
| 184 | 2-[(3R)-3-({1-[2-(difluoromethoxy)-4-methylphenyl]imidazo[1,5-d][1,2,4]triazin-4- |
| | yl}amino)piperidin-1-yl]ethan-1-ol formate |
| 185 | 2-[(3R)-3-({1-[4-cyclopropyl-2-(difluoromethoxy)phenyl]imidazo[1,5-d][1,2,4]triazin- |
| | 4-yl}amino)piperidin-1-yl]ethan-1-ol formate |
| 186 | 2-[(3R)-3-({1-[2-(difluoromethoxy)-4-(trifluoromethyl)phenyl]imidazo[1,5- |
| | d][1,2,4]triazin-4-yl}amino)piperidin-1-yl]ethan-1-ol formate |
| 190 | 1-[4-chloro-2-(difluoromethoxy)phenyl]-N-[(3R)-1-methylpiperidin-3-yl]imidazo[1,5- |
| | d][1,2,4]triazin-4-amine formate |
| 191 | 1-[4-chloro-2-(difluoromethoxy)phenyl]-N-[(3R)-1-ethylpiperidin-3-yl]imidazo[1,5- |
| | d][1,2,4]triazin-4-amine formate |
| 192 | 2-[(3R)-3-({1-[4-chloro-2-(difluoromethoxy)phenyl]imidazo[1,5-d][1,2,4]triazin-4- |
| | yl}amino)piperidin-1-yl]ethan-1-ol formate |
| 193 | 1-[4-chloro-2-(difluoromethoxy)phenyl]-N-[(3R,5R)-5-fluoro-1-methylpiperidin-3- |
| | yl]imidazo[1,5-d][1,2,4]triazin-4-amine formate |
| 216 | 1-[2-(difluoromethoxy)-4-methoxyphenyl]-N-[(3R)-1-methylpiperidin-3- |
| | yl]imidazo[1,5-d][1,2,4]triazin-4-amine formate |
| 228 | 1-[4-methoxy-2-(trifluoromethyl)phenyl]-N-(1H-pyrazol-3-yl)imidazo[1,5- |
| | d][1,2,4]triazin-4-amine formate |
| 250 | 1-[4-methoxy-2-(2,2,2-trifluoroethyl)phenyl]-N-[(3R)-1-methylpiperidin-3- |
| | yl]imidazo[1,5-d][1,2,4]triazin-4-amine formate |
| 261 | N-[(3R)-1-ethylpiperidin-3-yl]-1-[2-(2,2,2-trifluoroethyl)-4- |
| | (trifluoromethyl)phenyl]imidazo[1,5-d][1,2,4]triazin-4-amine formate |
| 278 | 2-[(3R)-3-({1-[4-methoxy-2-(2,2,2-trifluoroethyl)phenyl]imidazo[1,5-d][1,2,4]triazin-4- |
| | yl}amino)piperidin-1-yl]ethan-1-ol formate |
| 279 | 8-methyl-N-[(3R)-1-methylpiperidin-3-yl]-1-[2-(2,2,2-trifluoroethyl)-4- |
| | (trifluoromethyl)phenyl]imidazo[1,5-d][1,2,4]triazin-4-amine formate |
| 281 | 1-[4-methoxy-2-(pentafluoroethyl)phenyl]-N-[(3R)-1-methylpiperidin-3- |
| | yl]imidazo[1,5-d][1,2,4]triazin-4-amine formate |
| 317 | N-[(3R)-1-methylpiperidin-3-yl]-1-[4-methyl-2-(2,2,2- |
| | trifluoroethyl)phenyl]imidazo[1,5-d][1,2,4]triazin-4-amine formate |
| 357 | 4-[4-bromo-2-(trifluoromethoxy)phenyl]-N-[(3R)-1-ethylpiperidin-3-yl]-2- |
| | methylpyrazolo[1,5-d][1,2,4]triazin-7-amine formate |
| 358 | 4-[4-chloro-2-(difluoromethoxy)phenyl]-2-methyl-N-[(3R)-1-methylpiperidin-3- |
| | yl]pyrazolo[1,5-d][1,2,4]triazin-7-amine formate |
| 359 | 4-[2-(difluoromethyl)-4-(trifluoromethyl)phenyl]-2-methyl-N-[(3R)-1-methylpiperidin- |
| | 3-yl]pyrazolo[1,5-d][1,2,4]triazin-7-amine formate |
| 360 | 4-[4-cyclopropyl-2-(difluoromethoxy)phenyl]-2-methyl-N-[(3R)-1-methylpiperidin-3- |
| | yl]pyrazolo[1,5-d][1,2,4]triazin-7-amine formate |
| 361 | 4-[2-(difluoromethoxy)-4-methylphenyl]-2-methyl-N-[(3R)-1-methylpiperidin-3- |
| | yl]pyrazolo[1,5-d][1,2,4]triazin-7-amine formate |
| 363 | 4-[4-cyclopropyl-2-(difluoromethyl)phenyl]-2-methyl-N-[(3R)-1-methylpiperidin-3- |
| | yl]pyrazolo[1,5-d][1,2,4]triazin-7-amine formate |
| 364 | 4-[2-(difluoromethyl)-4-methylphenyl]-2-methyl-N-[(3R)-1-methylpiperidin-3- |
| | yl]pyrazolo[1,5-d][1,2,4]triazin-7-amine formate |
| 368 | N-[(3R)-1-ethylpiperidin-3-yl]-4-[2-(methanesulfonyl)-4-methylphenyl]-2- |
| | methylpyrazolo[1,5-d][1,2,4]triazin-7-amine trifluoroacetate |
| 369 | N-[(3R)-1-ethylpiperidin-3-yl]-4-[4-(methanesulfonyl)-2-(trifluoromethoxy)phenyl]-2- |
| | methylpyrazolo[1,5-d][1,2,4]triazin-7-amine trifluoroacetate |
| 372 | 2-(7-{[(3R)-1-ethylpiperidin-3-yl]amino}-2-methylpyrazolo[1,5-d][1,2,4]triazin-4-yl)- |
| | 5-(trifluoromethyl)benzamide formate |

wherein a form of the compound is selected from the group consisting of a hydrate, solvate, racemate, enantiomer, diastereomer, stereoisomer, tautomer, free-base and isotope enriched form thereof.

In embodiment 4, the invention provides a pharmaceutical composition comprising a therapeutically effective amount of a compound according to any one of embodiments 1 to 3 or a pharmaceutically acceptable salt thereof and one or more pharmaceutically acceptable carriers.

In embodiment 5, the invention provides a method for treating or ameliorating a disease modulated by NLRP3 in a subject in need thereof comprising, administering to the subject an effective amount of the compound according to any one of embodiments 1 to 3.

In embodiment 6, the invention provides a method of treating or ameliorating a disease modulated by NLRP3 according to embodiment 5 selected from Alzheimer disease, Frontotemporal dementia (FTD), Huntington's disease, Parkinson's disease, Perioperative neurocognitive disorders, Post-cardiac arrest cognitive impairment, Poststroke cognitive impairment, Sepsis, Sepsis associated encephalopathy, Subarachnoid hemorrhage, Macular Degeneration, Retinal neovascularization, Uveitis, Colitis, Endothelial dysfunction, Gout, Pseudogout, Graft-versus-host-disease (GvHD), Systemic lupus erythematosus-lupus nephritis, Cryopyrin-associated periodic syndromes (CAPS), Cystic fibrosis, Sickle-cell disease, VCP-associated disease, Liver fibrosis, Nonalcoholic fatty liver disease (NASH), muscle atrophy, inherited and acquired myopathies, e.g. Duchenne Muscular Dystrophy (DMD), Hyperalgesia, Multiple sclerosis-associated neuropathic pain, Acute Kidney Injury, Chronic crystal nephropathy, Chronic Kidney Disease, asthma and allergic airway inflammation Diabetes-associated atherosclerosis, Diabetic encephalopathy, Diabetic kidney disease, Islet transplantation rejection, Obesity-associated renal disease, Oxalate-induced nephropathy, Renal fibrosis, Renal hypertension, Type I diabetes, Type II diabetes, Psoriasis, Hidradenitis suppurativa, Atherosclerosis and Cytokine Release Syndrome (CRS).

In embodiment 7, the invention provides the method of any one of embodiments 5 to 6, wherein the effective amount of the compound is in a range of from about 0.001 mg/kg/day to about 500 mg/kg/day.

In embodiment 8, the invention provides a compound according to any one of embodiments 1 to 3 or a pharmaceutically acceptable salt thereof, for use in treating or ameliorating a disease modulated by NLRP3 selected from Alzheimer disease, Frontotemporal dementia (FTD), Huntington's disease, Parkinson's disease, Perioperative neurocognitive disorders, Post-cardiac arrest cognitive impairment, Poststroke cognitive impairment, Sepsis, Sepsis associated encephalopathy, Subarachnoid hemorrhage, Macular Degeneration, Retinal neovascularization, Uveitis, Colitis, Endothelial dysfunction, Gout, Pseudogout, Graft-versus-host-disease (GvHD), Systemic lupus erythematosus-lupus nephritis, Cryopyrin-associated periodic syndromes (CAPS), Cystic fibrosis, Sickle-cell disease, VCP-associated disease, Liver fibrosis, Nonalcoholic fatty liver disease (NASH), muscle atrophy, inherited and acquired myopathies, Hyperalgesia, Multiple sclerosis-associated neuropathic pain, Acute Kidney Injury, Chronic crystal nephropathy, Chronic Kidney Disease, asthma and allergic airway inflammation Diabetes-associated atherosclerosis, Diabetic encephalopathy, Diabetic kidney disease, Islet transplantation rejection, Obesity-associated renal disease, Oxalate-induced nephropathy, Renal fibrosis, Renal hypertension, Type I diabetes, Type II diabetes, Psoriasis, Hidradenitis suppurativa, Atherosclerosis and Cytokine Release Syndrome (CRS).

In embodiment 9, the invention provides a compound's use according to embodiment 8, where the effective amount of the compound is in a range from about 0.001 mg/kg/day to about 500 mg/kg/day.

In embodiment 10, the invention provides a use of a compound according to any one of embodiments 1 to 3 in the preparation of a pharmaceutical composition for treating or ameliorating a disease modulated by NLRP3 in a subject in need thereof comprising, administering to the subject an effective amount of the compound or a form thereof in admixture with one or more of the pharmaceutically acceptable excipients.

### Method of Use of the Invention

There is evidence for a role of NLRP3-induced IL-1β and IL-18 in the inflammatory responses occurring in connection with, or as a result of, a multitude of different disorders (Menu et al, Clinical and Experimental Immunology, 2011, 166, 1-15; Strowig et al, Nature, 2012, 481, 278-286). NLRP3 mutations have been found to be responsible for a set of rare autoinflammatory diseases known as CAPS (Ozaki et al, J. Inflammation Research, 2015, 8, 15-27; Schroder et al, Cell, 2010, 140:821-832; Menu et al, Clinical and Experimental Immunology, 2011, 166, 1-15). CAPS are heritable diseases characterized by recurrent fever and inflammation and are comprised of three autoinflammatory disorders that form a clinical continuum. These diseases, in order of increasing severity, are familial cold autoinflammatory syndrome (FCAS), Muckle-Wells syndrome (MWS), and chronic infantile cutaneous neurological articular syndrome (CINCA; also called neonatal-onset multisystem inflammatory disease, NOMID), and all have been shown to result from gain-of-function mutations in the NLRP3 gene, which leads to increased secretion of IL-I beta. NLRP3 has also been implicated in a number of autoinflammatory diseases, including pyogenic arthritis, pyoderma gangrenosum and acne (PAPA), Sweet's syndrome, chronic nonbacterial osteomyelitis (CNO), and acne vulgaris (Cook et al, Eur J. Immunol., 2010, 40, 595-653). A number of autoimmune diseases have been shown to involve NLRP3 including, in particular, multiple sclerosis, type-1 diabetes (TlD), psoriasis, rheumatoid arthritis (RA), Behcet's disease, Schnitzler syndrome, macrophage activation syndrome (Braddock et al. Nat. Rev. Drug Disc. 2004, 3, 1-10; Inoue et al, Immunology, 2013, 139, 11-18, Coll et al, Nat. Med. 2015, 21(3), 248-55; Scott et al, Clin. Exp. Rheumatol. 2016, 34(1), 88-93), systemic lupus erythematosus and its complications such as lupus nephritis (Lu et al, J. Immunol., 2017, 198(3), 1119-29), and systemic sclerosis (Artlett et al, Arthritis Rheum. 2011, 63(11), 3563-74). NLRP3 has also been shown to play a role in a number of lung diseases including chronic obstructive pulmonary disorder (COPD), asthma (including steroid resistant asthma), asbestosis, and silicosis (De Nardo et al, Am. J. Pathol., 2014, 184: 42-54; Kim et al. Am. J. Respir Crit Care Med, 2017, 196(3), 283-97). NLRP3 has also been suggested to have a role in a number of central nervous system conditions, including Multiple Sclerosis (MS), Parkinson's disease (PD), Alzheimer's disease (AD), dementia, Huntington's disease, cerebral malaria, brain injury from pneumococcal meningitis (Walsh et al, Nature Reviews, 2014, 15, 84-97; and Dempsey et al. Brain. Behav. Immun. 2017, 61, 306-16), intracranial aneurysms (Zhang et al. J. Stroke and Cerebrovascular Dis., 2015, 24, 5, 972-9), and traumatic brain injury (Ismael et al. J. Neurotrauma., 2018, 35(11), 1294-1303). NRLP3 activity has also been shown to be involved in various metabolic diseases including type 2 diabetes (T2D) and its organ-specific complications, atherosclerosis, obesity, gout, pseudo-gout, metabolic syndrome (Wen et al, Nature Immunology, 2012, 13, 352-357; Duewell et al, Nature, 2010, 464, 1357-1361; Strowig et al, Nature, 2014, 481, 278-286), and non-alcoholic steatohepatitis (Mridha et al. J. Hepatol. 2017, 66(5), 1037-46). NLRP3 is also suggested to play a key pathological role in the development and progression of several skeletal muscle diseases, e.g. muscle atrophy, inherited and acquired myopathies (Dubussion et al. Cell 2021, 10(11).3023). A role for NLRP3 via IL-I beta has also been suggested in atherosclerosis, myocardial infarction (van Hout et al. Eur Heart J. 2017, 38(11), 828-3-6), heart failure (Sano et al. J. Am. Coll. Cardiol. 2018, 71(8), 875-66), aortic aneurysm and dissection (Wu et al. Arterioscler Thromb. Vase. Biol., 2017, 37(4), 694-706), and other cardiovascular events (Ridker et al, N. Engl. J. Med, 2017, 377(12), 1119-31). Other diseases in which NLRP3 has been shown to be involved include: ocular diseases such as both wet and dry age-related macular degeneration (Doyle et al. Nature Medicine, 2012, 18, 791-798; Tarallo et al. Cell 2012, 149(4), 847-59), diabetic retinopathy (Loukovaara et al. Acta Ophthalmol., 2017, 95(8), 803-8), non-infectious uveitis and optic nerve damage (Puyang et al. Sci. Rep. 2016, 6, 20998); liver diseases including non-alcoholic steatohepatitis (NASH) and acute alcoholic hepatitis (Henao-Meija et al, Nature, 2012, 482, 179-185); inflammatory reactions in the lung and skin (Primiano et al. J. Immunol. 2016, 197(6), 2421-33) including contact hypersensitivity (such as bullous pemphigoid (Fang et al. J Dermatol Sci. 2016, 83(2),116-23)), atopic dermatitis (Niebuhr et al. Allergy, 2014, 69(8), 1058-67), Hidradenitis suppurativa (Alikhan et al. J. Am. Acad. Dermatol., 2009, 60(4), 539-61), and sarcoidosis (Jager et al. Am. J. Respir Crit. Care Med., 2015, 191, A5816); inflammatory reactions in the joints (Braddock et al, Nat. Rev. Drug Disc, 2004, 3, 1-10); amyotrophic lateral sclerosis (Gugliandolo et al. Int. J. Mol. Sci., 2018, 19(7), E1992); cystic fibrosis (Iarmitti et al. Nat. Commun., 2016, 7, 10791); stroke (Walsh et al, Nature Reviews, 2014, 15, 84-97); chronic kidney disease (Granata et al. PLoS One 2015, 10(3), eoi22272); and inflammatory bowel diseases including ulcerative colitis and Crohn's disease (Braddock et al, Nat. Rev. Drug Disc, 2004, 3, 1-10; Neudecker et al. J. Exp. Med. 2017, 214(6), 1737-52; Lazaridis et al. Dig. Dis. Sci. 2017, 62(9), 2348-56). The NLRP3 inflammasome has been found to be activated in response to oxidative stress. NLRP3 has also been shown to be involved in inflammatory hyperalgesia (Dolunay et al, Inflammation, 2017, 40, 3-66-86). US application US202003-61898 in incorporated herein by reference.

### Definitions

To assist in understanding the scope of the compounds of Formula I or a form thereof described herein, the following Specific Examples are included. The experiments relating to the compounds of Formula I or a form thereof described herein should not, of course, be construed as specifically limiting the scope of the compounds of Formula I or a form thereof described herein and such variations of the compounds of Formula I or a form thereof as described herein, now known or later developed, which would be within the purview of one skilled in the art are considered to fall within the scope as described herein and hereinafter claimed.

Other than in the working examples, unless indicated to the contrary, all numbers expressing quantities of ingredients, reaction conditions, experimental data, and so forth used in the specification and claims are to be understood as being modified by the term “about”. Accordingly, all such numbers represent approximations that may vary depending upon the desired properties sought to be obtained by a reaction or as a result of variable experimental conditions. Therefore, within an expected range of experimental reproducibility, the term “about” in the context of the resulting data, refers to a range for data provided that may vary according to a standard deviation from the mean. As well, for experimental results provided, the resulting data may be rounded up or down to present data consistently, without loss of significant figures. At the very least, and not as an attempt to limit the application of the doctrine of equivalents to the scope of the claims, each numerical parameter should be construed in light of the number of significant digits and ordinary rounding techniques.

While the numerical ranges and parameters setting forth the characterization of the compounds of Formula I or a form thereof described herein are approximations, the numerical values set forth in the working examples are reported as precisely as possible. Any numerical value, however, inherently contains certain errors necessarily resulting from the standard deviation found in their respective testing measurements.

The compounds of Formula I or a form thereof provided herein are described in more detail with reference to the following non-limiting examples, which are offered to more fully illustrate the scope of the compounds of Formula I or a form thereof described herein but are not to be construed as limiting the scope thereof. The examples illustrate the preparation of compounds of Formula I or a form thereof described herein, and the testing of these compounds of Formula I or a form thereof in vitro and/or in vivo. Those of skill in the art will understand that the synthesis techniques described in these examples represent techniques that fall within the practice of those having ordinary skill in the chemical arts, and as such constitute preferred modes for the practice thereof. However, it should be appreciated that those having skill in the art should, in light of the present disclosure, appreciate that many changes can be made in the specific methods that are disclosed herein while still obtaining a like or similar result without departing from the spirit and scope described herein.

In certain embodiments described herein, the compound of Formula I or a form thereof is isolated for use.

As used herein, the term “isolated” means the physical state of a compound of Formula I or a form thereof after being isolated and/or separated and/or purified from a synthetic process (e.g., from a reaction mixture) or natural source or combination thereof according to an isolation, separation or purification process or processes described herein or which are well known to the skilled artisan (e.g., chromatography, recrystallization and the like) in sufficient purity to be characterizable by standard analytical techniques described herein or well known to the skilled artisan.

As used herein, the term “protected” means that a functional group on a compound of Formula I or a form thereof is in a form modified to preclude undesired side reactions of the functional group when the compound is subjected to a reaction. Suitable protecting groups will be recognized by those with ordinary skill in the art as well as by reference to standard textbooks such as, for example, T. W. Greene et al, Protective Groups in Organic Synthesis (2007), Wiley, New York.

Prodrugs and solvates of the compounds of Formula I or a form thereof described herein are also contemplated.

As used herein, the term “prodrug” means that a functional group on a compound of Formula I or a form thereof is in a form (e.g., acting as an active or inactive drug precursor) that is transformed in vivo to yield an active or more active compound of Formula I or a form thereof. The transformation may occur by various mechanisms (e.g., by metabolic and/or nonmetabolic chemical processes), such as, for example, by hydrolysis and/or metabolism in blood, liver and/or other organs and tissues. A discussion of the use of prodrugs is provided by V. J. Stella, et. al., “Biotechnology: Pharmaceutical Aspects, Prodrugs: Challenges and Rewards,” American Association of Pharmaceutical Scientists and Springer Press, 2007.

In one example, when a compound of Formula I or a form thereof contains a carboxylic acid functional group, a prodrug can comprise an ester formed by the replacement of the hydrogen atom of the acid group with a functional group such as alkyl and the like. In another example, when a compound of Formula I or a form thereof contains an alcohol functional group, a prodrug can be formed by the replacement of the hydrogen atom of the alcohol group with a functional group such as alkyl or carbonyloxy and the like. In another example, when a compound of Formula I or a form thereof contains an amine functional group, a prodrug can be formed by the replacement of one or more amine hydrogen atoms with a functional group such as alkyl or substituted carbonyl.

Pharmaceutically acceptable prodrugs of compounds of Formula I or a form thereof include those compounds substituted with one or more of the following groups: carboxylic acid esters, sulfonate esters, amino acid esters, phosphonate esters (e.g., a phosphoramidic acid used to derive a phosphoramidic acid) and mono-, di- or triphosphate esters further substituted with alkyl, where appropriate. As described herein, it is understood by a person of ordinary skill in the art that one or more of such substituents may be used to provide a compound of Formula I or a form thereof as a prodrug.

The compounds of Formula I or a form thereof can form salts, which are intended to be included within the scope of this description. Reference to a compound of Formula I or a form thereof is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula I or a form thereof contains both a basic moiety, such as, but not limited to a pyridine or imidazole, and an acidic moiety, such as, but not limited to a carboxylic acid, zwitterions (“inner salts”) may be formed and are included within the term “salt(s)” as used herein.

The term “pharmaceutically acceptable salt(s)”, as used herein, means those salts of compounds of Formula I or a form thereof described herein that are safe and effective (i.e., non-toxic, physiologically acceptable) for use in mammals and that possess biological activity, although other salts are also useful. Salts of the compounds of the Formula I or a form thereof may be formed, for example, by reacting a compound of Formula I or a form thereof and compounds described herein with an amount of acid or base, such as an equivalent amount, in a medium such as one in which the salt precipitates or in an aqueous medium followed by lyophilization.

Additionally, acids which are considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33, 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Suitable basic salts include, but are not limited to, aluminum, ammonium, calcium, lithium, magnesium, potassium, sodium, zinc, and diethanolamine salts. Certain compounds of Formula I or a form thereof described herein can also form pharmaceutically acceptable salts with organic bases (for example, organic amines) such as, but not limited to, dicyclohexylamines, tert-butyl amines and the like, and with various amino acids such as, but not limited to, arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be included within the scope of pharmaceutically acceptable salts as described herein. In addition, all such acid and base salts are considered equivalent to the free forms of the corresponding compounds for purposes of this description.

Compounds of Formula I, and forms thereof, may further exist in a tautomeric form. All such tautomeric forms are contemplated and intended to be included within the scope of the compounds of Formula I or a form thereof as described herein.

The compounds of Formula I or a form thereof may contain asymmetric or chiral centers, and, therefore, may exist in different stereoisomeric forms. The present description is intended to include all stereoisomeric forms of the compounds of Formula I or a form thereof, as well as mixtures thereof, including racemic mixtures.

The compounds of Formula I or a form thereof described herein may include one or more chiral centers, and as such may exist as racemic mixtures (R/S) or as substantially pure enantiomers and diastereomers. The compounds may also exist as substantially pure (R) or (S) enantiomers (when one chiral center is present). In one embodiment, the compounds of Formula I or a form thereof described herein are (S) isomers and may exist as enantiomerically pure compositions substantially comprising only the (S) isomer. In another embodiment, the compounds of Formula I or a form thereof described herein are (R) isomers and may exist as enantiomerically pure compositions substantially comprising only the (R) isomer. As one of skill in the art will recognize, when more than one chiral center is present, the compounds of Formula I or a form thereof described herein may also exist as a (R,R), (R,S), (S,R) or (S,S) isomer, as defined by IUPAC Nomenclature Recommendations.

As used herein, the term “substantially pure” refers to compounds of Formula I or a form thereof consisting substantially of a single isomer in an amount greater than or equal to 90%, in an amount greater than or equal to 92%, in an amount greater than or equal to 95%, in an amount greater than or equal to 98%, in an amount greater than or equal to 99%, or in an amount equal to 100% of the single isomer.

In one aspect of the description, a compound of Formula I or a form thereof is a substantially pure (S) enantiomer present in an amount greater than or equal to 90%, in an amount greater than or equal to 92%, in an amount greater than or equal to 95%, in an amount greater than or equal to 98%, in an amount greater than or equal to 99%, or in an amount equal to 100%.

In one aspect of the description, a compound of Formula I or a form thereof is a substantially pure (R) enantiomer present in an amount greater than or equal to 90%, in an amount greater than or equal to 92%, in an amount greater than or equal to 95%, in an amount greater than or equal to 98%, in an amount greater than or equal to 99%, or in an amount equal to 100%.

As used herein, the term “racemate” refers to any mixture of isometric forms that are not “enantiomerically pure”, including mixtures such as, without limitation, in a ratio of about 50/50, about 60/40, about 70/30, or about 80/20, about 85/15 or about 90/10.

In addition, the compounds of Formula I or a form thereof described herein embrace all geometric and positional isomers. For example, if a compound of Formula I or a form thereof incorporates a double bond or a fused ring, both the cis- and trans-forms, as well as mixtures thereof, are embraced within the scope of the compounds of Formula I or a form thereof described herein.

Diastereomeric mixtures can be separated into their individual diastereomers on the basis of their physical chemical differences by methods well known to those skilled in the art, such as, for example, by chromatography and/or fractional crystallization. Enantiomers can be separated by use of a chiral HPLC column or other chromatographic methods known to those skilled in the art.

Enantiomers can also be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers.

All stereoisomers (for example, geometric isomers, optical isomers and the like) of the present compounds of Formula I or a form thereof (including salts, solvates, esters and prodrugs and transformed prodrugs thereof), which may exist due to asymmetric carbons on various substituents, including enantiomeric forms (which may exist even in the absence of asymmetric carbons), rotameric forms, atropisomers, diastereomeric and regioisomeric forms, are contemplated within the scope of the description herein. Individual stereoisomers of the compounds of Formula I or a form thereof described herein may, for example, be substantially free of other isomers, or may be present in a racemic mixture, as described supra.

The use of the terms “salt,” “solvate,” “ester,” “prodrug” and the like, is intended to apply equally to the salt, solvate, ester and prodrug of enantiomers, stereoisomers, rotamers, tautomers, positional isomers, racemates, isotope enriched or prodrugs of the instant compounds.

One or more compounds of Formula I or a form thereof described herein may exist in unsolvated as well as solvated forms with pharmaceutically acceptable solvents such as water, ethanol, and the like, and the description herein is intended to embrace both solvated and unsolvated forms.

As used herein, the term “solvate” means a physical association of a compound of Formula I or a form thereof described herein with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. As used herein, “solvate” encompasses both solution-phase and isolatable solvates. Non-limiting examples of suitable solvates include ethanolates, methanolates, and the like.

One or more compounds of Formula I or a form thereof described herein may optionally be converted to a solvate. Preparation of solvates is generally known. A typical, non-limiting process involves dissolving a compound of Formula I or a form thereof in a desired amount of the desired solvent (organic or water or mixtures thereof) at a higher than ambient temperature and cooling the solution at a rate sufficient to form crystals which are then isolated by standard methods. Analytical techniques such as, for example infrared spectroscopy, show the presence of the solvent (or water) in the crystals as a solvate (or hydrate).

As used herein, the term “hydrate” means a solvate wherein the solvent molecule is water.

Polymorphic crystalline and amorphous forms of the compounds of Formula I or a form thereof, and of the salts, solvates, esters and prodrugs of the compounds of Formula I or a form thereof, are further intended to be included in the scope of the compounds of Formula I or a form thereof described herein.

As used herein, the term “isotope enriched” means a compounds of Formula I or a form thereof which are identical to those recited herein, but for the fact that one or more atoms are replaced by an atom having an atomic mass or mass number different from the atomic mass or mass number usually found in nature. Examples of isotopes that can be incorporated into compounds of Formula I or a form thereof described herein include isotopes of hydrogen, carbon, nitrogen, oxygen, phosphorus, fluorine and chlorine, such as H², H³, C¹³, C¹⁴, N¹⁵, O¹⁸, O¹⁷, P³¹, P³², ³⁵, F¹⁸, Cl³⁵ and Cl³⁶, respectively, each of which is also within the scope of this description.

### Dosage and Administration

The compounds of the present invention may be formulated in a wide variety of oral administration dosage forms and carriers. Oral administration can be in the form of tablets, coated tablets, dragées, hard and soft gelatine capsules, solutions, emulsions, syrups, or suspensions. Compounds of the present invention are efficacious when administered by other routes of administration including continuous (intravenous drip) topical parenteral, intramuscular, intravenous, subcutaneous, transdermal (which may include a penetration enhancement agent), buccal, nasal, inhalation and suppository administration, among other routes of administration. The preferred manner of administration is generally oral using a convenient daily dosing regimen which can be adjusted according to the degree of affliction and the patient's response to the active ingredient.

A compound or compounds of the present invention, as well as their pharmaceutically useable salts, together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. A typical preparation will contain from about 5% to about 95% active compound or compounds (w/w). The term “preparation” or “dosage form” is intended to include both solid and liquid formulations of the active compound and one skilled in the art will appreciate that an active ingredient can exist in different preparations depending on the target organ or tissue and on the desired dose and pharmacokinetic parameters.

The term “excipient” as used herein refers to a compound that is useful in preparing a pharmaceutical composition, generally safe, non-toxic and neither biologically nor otherwise undesirable, and includes excipients that are acceptable for veterinary use as well as human pharmaceutical use. The compounds of this invention can be administered alone but will generally be administered in admixture with one or more suitable pharmaceutical excipients, diluents or carriers selected with regard to the intended route of administration and standard pharmaceutical practice.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

A “pharmaceutically acceptable salt” form of an active ingredient may also initially confer a desirable pharmacokinetic property on the active ingredient which were absent in the non-salt form, and may even positively affect the pharmacodynamics of the active ingredient with respect to its therapeutic activity in the body. The phrase “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well known suspending agents.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the present invention may be formulated for vaginal administration. Pessaries, tampons, creams, gels, pastes, foams or sprays containing in addition to the active ingredient such carriers as are known in the art to be appropriate.

The compounds of the present invention may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the present invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to an skin-adhesive solid support. The compound of interest can also be combined with a penetration enhancer, e.g., Azone (1-dodecylaza-cycloheptan-2-one). Sustained release delivery systems are inserted subcutaneously into to the subdermal layer by surgery or injection. The subdermal implants encapsulate the compound in a lipid soluble membrane, e.g., silicone rubber, or a biodegradable polymer, e.g., polyactic acid.

Suitable formulations along with pharmaceutical carriers, diluents and excipients are described in Remington: The Science and Practice of Pharmacy 1995, edited by E. W. Martin, Mack Publishing Company, 19th edition, Easton, Pennsylvania. A skilled formulation scientist may modify the formulations within the teachings of the specification to provide numerous formulations for a particular route of administration without rendering the compositions of the present invention unstable or compromising their therapeutic activity.

The modification of the present compounds to render them more soluble in water or other vehicle, for example, may be easily accomplished by minor modifications (salt formulation, esterification, etc.), which are well within the ordinary skill in the art. It is also well within the ordinary skill of the art to modify the route of administration and dosage regimen of a particular compound in order to manage the pharmacokinetics of the present compounds for maximum beneficial effect in patients.

The term “therapeutically effective amount” as used herein means an amount required to reduce symptoms of the disease in an individual. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.01 and about 1000 mg/kg body weight per day should be appropriate in monotherapy and/or in combination therapy. A preferred daily dosage is between about 0.1 and about 500 mg/kg body weight, more preferred 0.1 and about 100 mg/kg body weight and most preferred 1.0 and about 10 mg/kg body weight per day. Thus, for administration to a 70 kg person, the dosage range would be about 7 mg to 0.7 g per day. The daily dosage can be administered as a single dosage or in divided dosages, typically between 1 and 5 dosages per day. Generally, treatment is initiated with smaller dosages which are less than the optimum dose of the compound. Thereafter, the dosage is increased by small increments until the optimum effect for the individual patient is reached. One of ordinary skill in treating diseases described herein will be able, without undue experimentation and in reliance on personal knowledge, experience and the disclosures of this application, to ascertain a therapeutically effective amount of the compounds of the present invention for a given disease and patient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Examples of representative compounds encompassed by the present invention and within the scope of the invention are provided in the following Table. These examples and preparations which follow are provided to enable those skilled in the art to more clearly understand and to practice the present invention. They should not be considered as limiting the scope of the invention, but merely as being illustrative and representative thereof.

In general, the nomenclature used in this Application is based on AUTONOMTM v. 4.0, a Beilstein Institute computerized system for the generation of IUPAC systematic nomenclature. If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

The reagents and solvents were used as purchased (from a variety of vendors), except where noted. Where applicable, the term “Celite” is used as shown in the following examples to represent the tradename CELITE® (brand of diatomaceous earth). Where applicable, chromatographic separations were performed using techniques and equipment commonly available such as, for example, by using an ISCO CombiFlash® Rf system. Where applicable, NMR spectra were obtained using techniques and equipment commonly available such as, for example, by using a Bruker Avance III⁵⁰⁰ spectrometer with deuterated solvents such as, for example, DMSO-d₆ or residual solvent as standard. Where applicable, melting points were determined using techniques and equipment commonly available such as, for example, by using a SRS OptiMelt® MPA100 (values as obtained without correction/calibration). Where applicable, TLC analysis was performed using techniques and equipment commonly available such as, for example, by using Aldrich 254 nm glass-backed plates (60 Å, 250 m), visualized using UV and I₂ stains. Where applicable, ESI mass spectra were obtained using techniques and equipment commonly available such as, for example, by using an ACQUITY UPLC® System, with values shown as [M+H]⁺ or [M−H]⁻, unless otherwise indicated. Where applicable, the structure of the product was obtained via a 2D NOESY (Nuclear Overhauser SpectroscopY) experiment.

### Compounds and Preparation

Examples of representative compounds encompassed by the present invention and within the scope of the invention are provided herein. These examples and preparations which follow are provided to enable those skilled in the art to more clearly understand and to practice the present invention. They should not be considered as limiting the scope of the invention, but merely as being illustrative and representative thereof.

### Experimental

As disclosed herein, general methods for preparing the compounds of Formula I or a form thereof as described herein can be prepared using the methods summarized in Schemes A-J by the suitable selection of reagents with appropriate substitution, solvents, temperatures, pressures, and other reaction conditions readily selected by one of ordinary skilled in the art. Many of the starting materials are commercially available or, when not available, can be prepared via standard, well-known synthetic methodology or using the routes described below using techniques known to those skilled in the art. The synthetic schemes provided herein comprise multiple reaction steps, each of which is intended to stand on its own and can be carried out with or without any preceding or succeeding step(s). In other words, each of the individual reaction steps of the synthetic schemes provided herein in isolation is contemplated.

Depending on the nature of the groups depicted in the schemes below, the final compounds or their precursors may be further elaborated using the standard, well-known synthetic methods such as SN₂ displacement reaction, metal catalyzed coupling reactions like Suzuki coupling, Negishi coupling and Buchwald coupling, reductive amination, etc. to afford the compounds of the general Formula I-II.

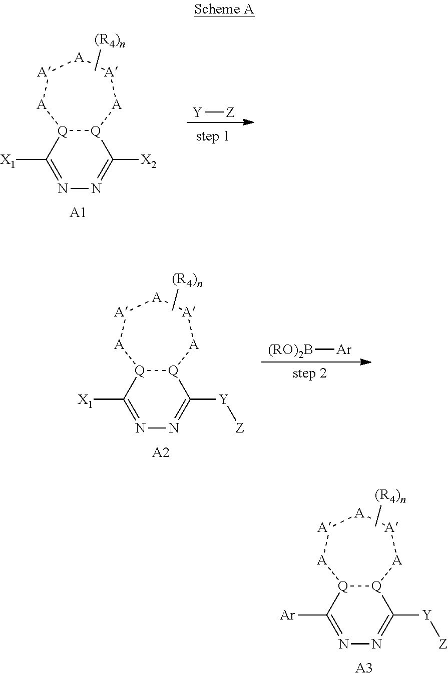

Compound A1 (where X₁ and X₂ are independently bromine, chlorine and the like) is converted to Compound A2 by a nucleophilic substitution with either an appropriate amine in the presence of a suitable base (such as DIEPA and the like) in a suitable solvent (such as NMP and the like) or with an appropriate alcohol in the presence of a suitable base (such as NaH and the like) in a suitable solvent (such as anhydrous THE and the like). Compound A2 is converted to Compound A3 by a Suzuki coupling with an aryl- or heteroaryl-boronic acid (or pinacol boronic ester) in the presence of a catalyst (such as Pd(dppf)Cl₂ and the like) and base (such as aqueous K₂CO₃ and the like) in a suitable solvent (such as 1,4-dioxane and the like).

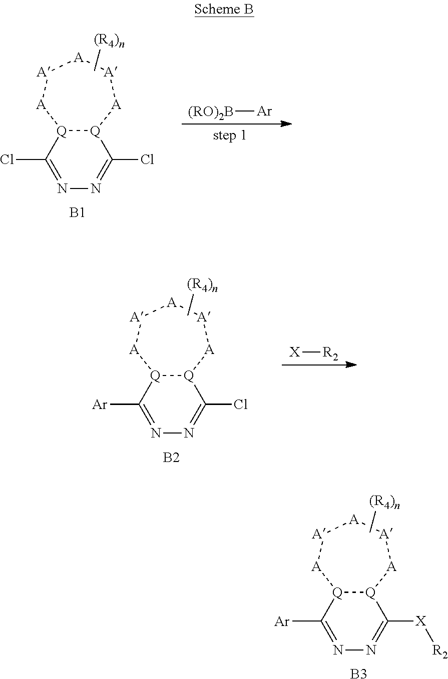

Compound B1 is converted to Compound B2 by a Suzuki coupling with an aryl- or heteroaryl-boronic acid (or pinacol boronic ester) in the presence of a catalyst (such as Pd(dppf)Cl₂ and the like) and base (such as aqueous K₂CO₃ and the like) in a suitable solvent (such as 1,4-dioxane and the like). Compound B2 is converted to Compound B3 by a Buchwald-Hartwig coupling with an appropriate amine in the present of a catalyst (such as Pd₂(dba)₃ and the like), a ligand (such as RuPhos and the like) and a base (such as NaO^{t}Bu and the like) in a suitable solvent (such as PhMe and the like).

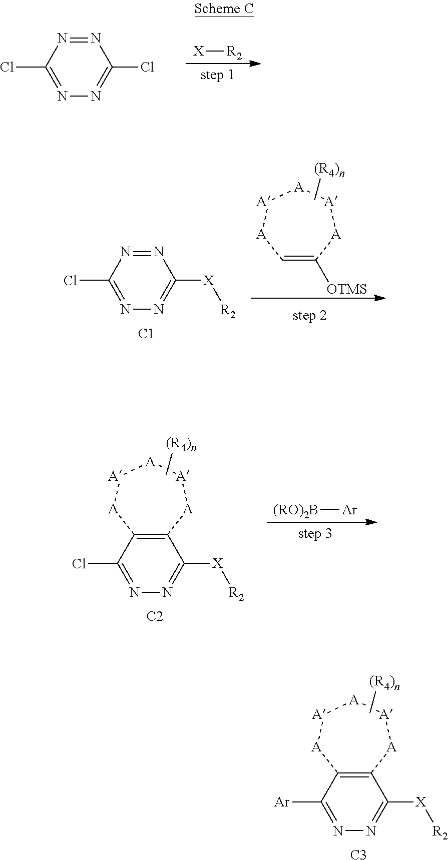

Compound C1 is prepared from 1,2,4,5-tetrazines with an appropriate amine in the presence of a suitable base (such as DIEPA and the like) in a suitable solvent (such as DCM and the like). Compound C1 is converted to Compound C2 by inverse electron demand Diels-Alder reaction with an appropriate enol ethers or enamine in a suitable solvent (such as PhMe and the like). Following conditions described in Scheme A—step 2, compound C2 was converted to compound C3.

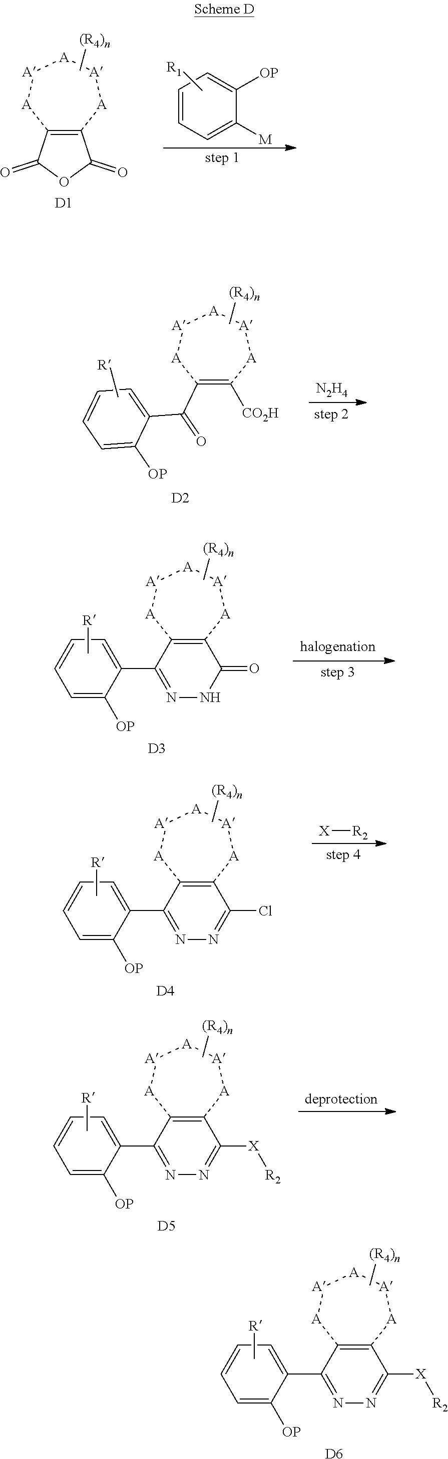

Compound D1 is converted to Compound D2 (where P is a protecting group such as Me and the like) by reacting with an appropriate organometallic compound (such as Grignard reagent and the like) in a suitable solvent (such as THE and the like). Compound D2 is converted to Compound D3 by condensation/cyclization sequence in the present of hydrazine in a suitable solvent (such as EtOH and the like). Compound D3 is converted to Compound D4 by treatment with a dehydrative halogenating agent (such as POCl₃ and the like). Compound D4 is converted to Compound D5 by a Buchwald-Hartwig coupling with an appropriate amine in the present of a catalyst (such as Pd₂(dba)₃ and the like), a ligand (such as RuPhos and the like) and a base (such as NaO^{t}Bu and the like) in a suitable solvent (such as PhMe and the like). Compound D5 is converted to Compound D6 upon treatment with conditions appropriate to removal of the protection groups (such as BBr₃ in DCM for a Me protection group in suitable solvent (such as DCM and the like).

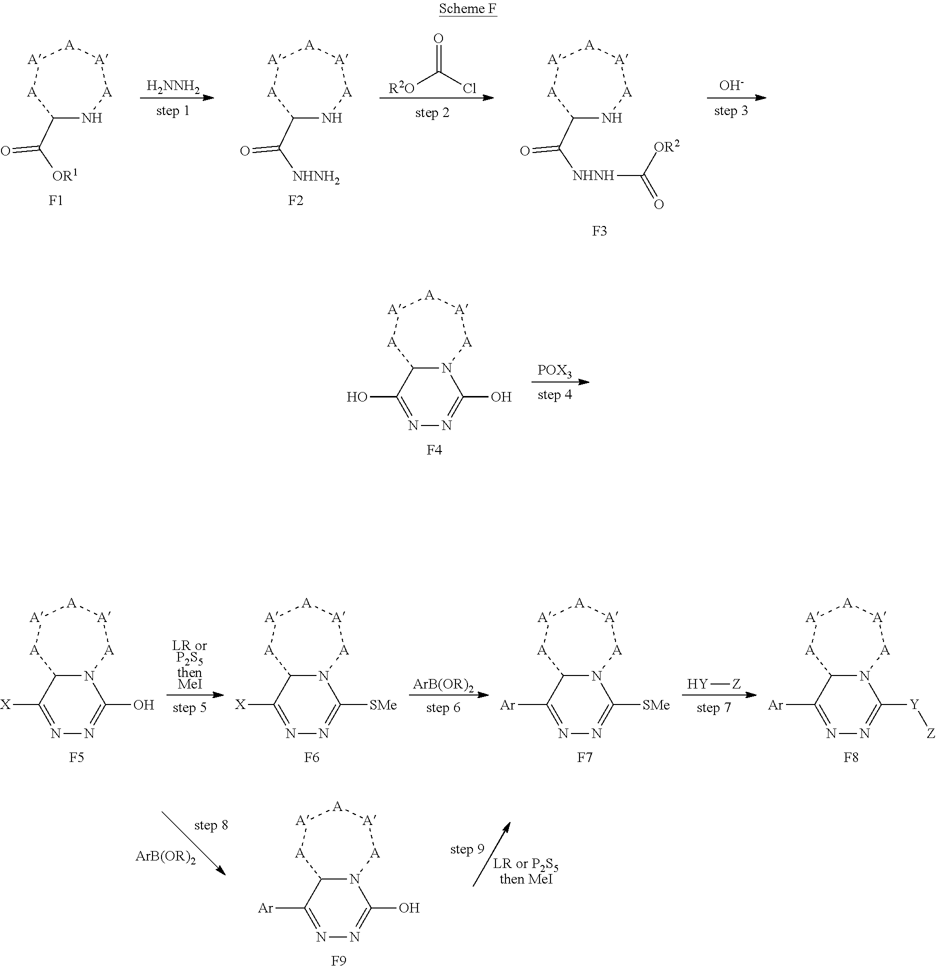

Compound F1 is converted to compound F2 by reacting with hydrazine in a suitable solvent (such as EtOH and the like). Reaction of F2 with chloroformate in the presence of a base (such as DIPEA and the like) in a suitable solvent (such as DCM and the like) provides F3, which is cyclized to F4 by treating with a base (such as KOH and the like) in a suitable solvent (such as EtOH and the like) at an elevated temperature (such as 80° C. and the like). Compound F4 is converted to compound F5 by treating with POX₃ (X═Cl or Br) with or without a base (such as DIPEA and the like). Treatment of F5 with a thionating reagent such as Lawesson's Reagent (LR) or P₂S₅ at an appropriate temperature such as 100° C., followed by alkylation with Mel provides F6. Compound F6 is converted to F7 by Suzuki coupling with an aryl or hetero boronic acid or borate in the presence of a suitable catalyst (such as PdCl₂dppf and the like) and a base (such as K₂CO₃ and the like) in a suitable solvent (such as dioxane and the like). Alternatively, compound F5 is converted to compound F7 by a Suzuki coupling first to give compound F9, followed by thionation with LR or P₂S₅ and subsequent alkylation with Mel. SNAr reaction of F7 with a nucleophile in a suitable solvent (such as DMSO and the like) at an elevated temperature (such as 130° C. and the like) provides F8.

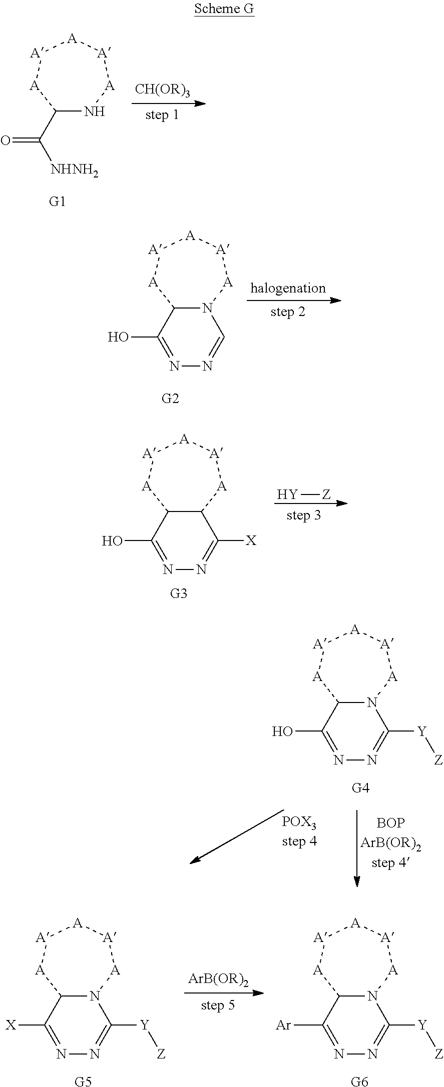

Compound G1, prepared according to step 1 in scheme F, is converted to compound G2 by reacting with tri-alkoxy orthoformate in a suitable solvent (such as EtOH and the like) at an elevated temperature (such as 100° C. and the like). Reaction of G2 with a halogenation reagent (such as NBS and the like) in a suitable solvent (such as DMF and the like) provides G3, which is reacted with a nucleophile to give the compound G4. Compound G4 is converted to compound G5 by treating with POX₃ (X═Cl or Br) with or without a base (such as DIPEA and the like) at an elevated temperature (such as 100° C. and the like). Suzuki coupling of compound G5 with an aryl or heteroaryl boronic acid or borate in the presence of a suitable catalyst (such as PdCl₂dppf and the like) and a base (such as K₂CO₃ and the like) in a suitable solvent (such as dioxane and the like) provides G6. Alternatively, compound G4 can be converted to compound G6 directly by a BOP-medicated Suzuki coupling.

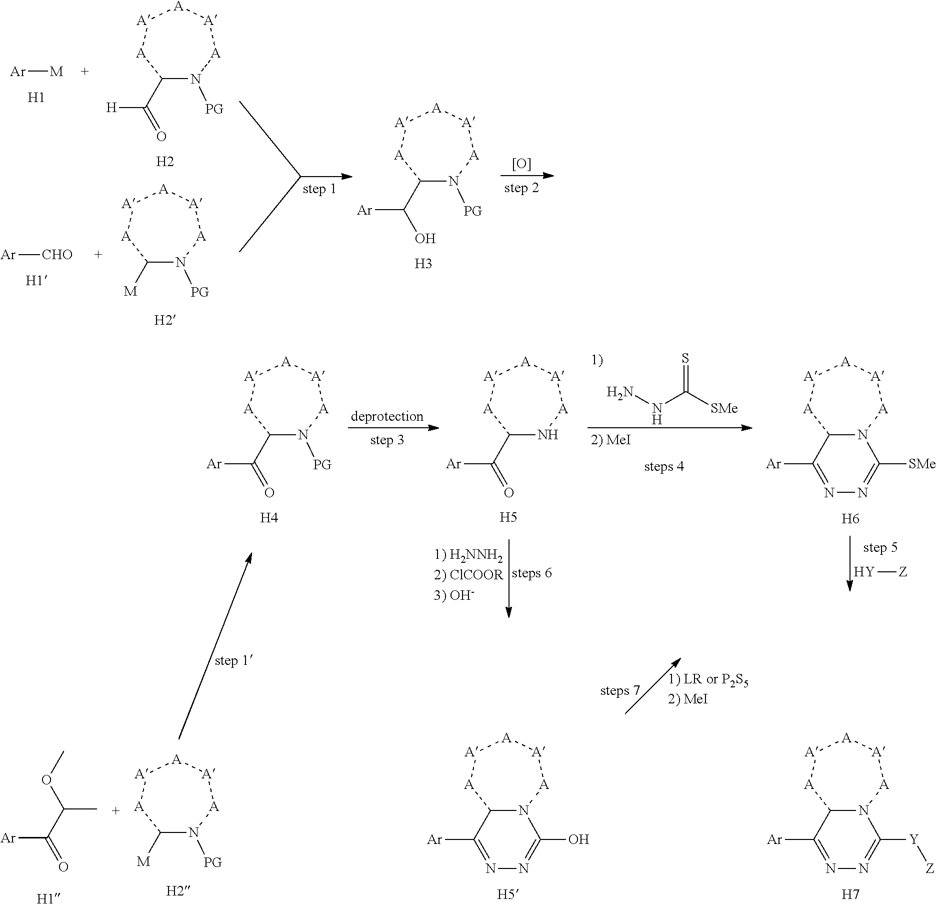

Reaction of an organometallic compound with an aldehyde, either H1 with H2, or H1′ with H2′, affords the alcohol H3. Compound H3 is converted to H4 by treating with an oxidant (such as MnO₂ and the like) in a suitable solvent (such as DCM and the like). Alternatively, reaction of compounds H1″ with H2″ yields H4 directly. Deprotection of compound H4 provides compound H5. Reaction of compound H5 with methyl hydrazinecarbodithioate in a suitable solvent (such as EtOH and the like) at an elevated temperature (such as 80° C. and the like) followed by alkylation with Mel in the presence of a base (such as K₂CO₃ and the like) provides compound H6. Alternatively, compound H5 is converted to compound H5′ by reacting with hydrazine in a suitable solvent (such as EtOH and the like), followed by reaction with chloroformate in the presence of a base (such as DIPEA and the like) in a suitable solvent (such as DCM and the like) and cyclization by treating with a base (such as KOH and the like) in a suitable solvent (such as EtOH and the like) at an elevated temperature (such as 80° C. and the like). SNAr reaction of H6 with a nucleophile in a suitable solvent (such as DMSO and the like) at an elevated temperature (such as 130° C. and the like) provides H7.

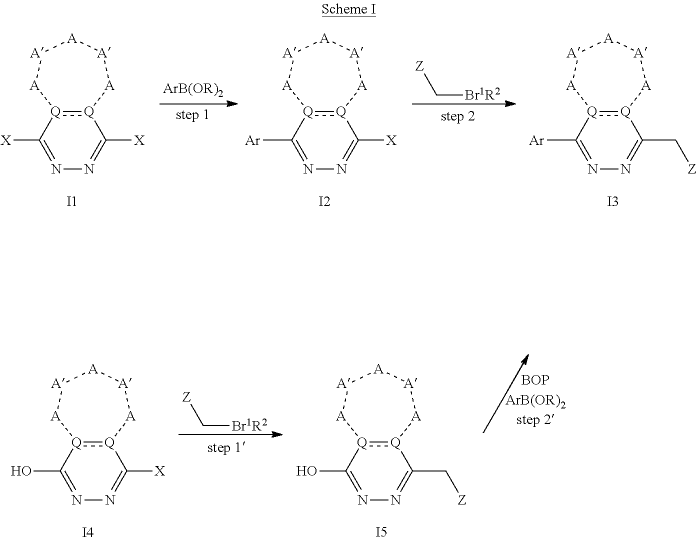

Compound I1 is converted to compound I3 by two coupling reactions with boronic acids or borates in the presence of a suitable catalyst (such as PdCl₂dppf and the like) and a base (such as K₂CO₃ and the like) in a suitable solvent (such as dioxane and the like). Alternatively, compound I4 is converted to compound I5 by coupling with a boronic acid or borate, which is further converted compound I3 by a BOP-mediated Suzuki coupling with an aryl or heteroaryl boronic acid or borate.

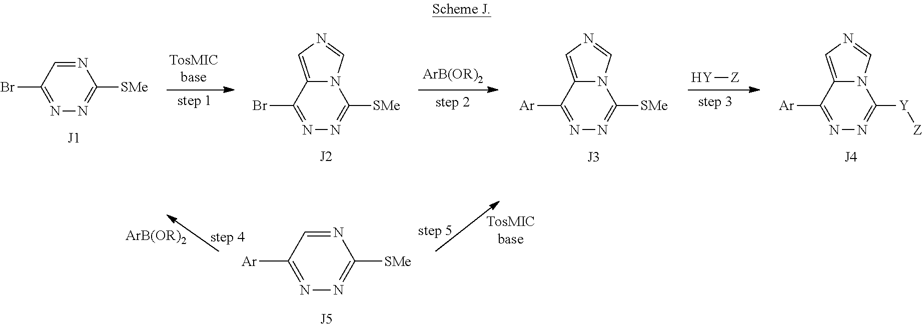

Compound J1 is converted to compound J2 by reacting with TosMIC in the presence of a suitable base (such as K₂CO₃, DBU and the like) in a suitable solvent (such as DCE and the like). Suzuki coupling of compound J2 with an aryl or heteroaryl boronic acid or borate in the presence of a suitable catalyst (such as PdCl₂dppf and the like) and a base (such as K₂CO₃ and the like) in a suitable solvent (such as dioxane and the like) provides J3. Alternatively, compound J1 is converted to compound J5 by coupling with a boronic acid or borate, which is further converted to compound J3 by reacting with TosMIC in the presence of a suitable base (such as K₂CO₃, DBU and the like) in a suitable solvent (such as DCE and the like). SNAr reaction of J3 with a nucleophile in a suitable solvent (such as DMSO and the like) at an elevated temperature (such as 130° C. and the like) provides J4.

### SYNTHETIC EXAMPLES

To describe in more detail and assist in understanding, the following non-limiting examples are offered to more fully illustrate the scope of compounds described herein and are not to be construed as specifically limiting the scope thereof. Such variations of the compounds described herein that may be now known or later developed, which would be within the purview of one skilled in the art to ascertain, are considered to fall within the scope of the compounds as described herein and hereinafter claimed. These examples illustrate the preparation of certain compounds. Those of skill in the art will understand that the techniques described in these examples represent techniques, as described by those of ordinary skill in the art, that function well in synthetic practice, and as such constitute preferred modes for the practice thereof. However, it should be appreciated that those of skill in the art should, in light of the present disclosure, appreciate that many changes can be made in the specific methods that are disclosed and still obtain a like or similar result without departing from the spirit and scope of the present description.

Other than in the following examples of the embodied compounds, unless indicated to the contrary, all numbers expressing quantities of ingredients, reaction conditions, experimental data, and so forth used in the specification and claims are to be understood as being modified by the term “about”. Accordingly, all such numbers represent approximations that may vary depending upon the desired properties sought to be obtained by a reaction or as a result of variable experimental conditions. Therefore, within an expected range of experimental reproducibility, the term “about” in the context of the resulting data, refers to a range for data provided that may vary according to a standard deviation from the mean. As well, for experimental results provided, the resulting data may be rounded up or down to present data consistently, without loss of significant figures. At the very least, and not as an attempt to limit the application of the doctrine of equivalents to the scope of the claims, each numerical parameter should be construed in light of the number of significant digits and rounding techniques used by those of skill in the art.

While the numerical ranges and parameters setting forth the broad scope of the present description are approximations, the numerical values set forth in the examples set forth below are reported as precisely as possible. Any numerical value, however, inherently contains certain errors necessarily resulting from the standard deviation found in their respective testing measurements.

### COMPOUND EXAMPLES

As used above, and throughout the present description, the following abbreviations, unless otherwise indicated, shall be understood to have the following meanings:

| Abbreviation | Meaning |
|---|---|
| 9-BBN | 9-borabicyclo[3.3.1]nonane |
| AcOH or HOAc | acetic acid |
| ACN or MeCN | acetonitrile |
| Ar | argon gas |
| aq. | aqueous |
| BBr₃ | boron tribromide |
| BippyPhos | 5-(Di-tert-butylphosphino)-1′,3′,5′-triphenyl-1′H- |
| | [1,4′]bipyrazole |
| BOP | benzotriazol-1-yloxytris(dimethylamino)phosphonium |
| | hexafluorophosphate |
| Br₂ | bromine |
| nBuLi | n-butyl lithium |
| CaCO₃ | calcium carbonate |
| Cbz—Cl | benzyl chloroformate or benzyl chlorocarbonate or Z-chloride |
| CDCl₃ | deuterated chloroform or chloroform-d |
| Celite | diatomaceous earth |
| CH₂Cl₂ or DCM | dichloromethane |
| m-CPBA | meta-chloroperoxybenzoic acid |
| Cs₂CO₃ | cesium carbonate |
| CO | carbon monoxide |
| CuI | copper(I) iodide |
| DAST | diethylaminosulfur trifluoride |
| DBU | 1,8-diazabicyclo[5.4.0]undec-7-ene |
| DCE | 1,2-dichloroethane or ethylene dichloride |
| DIEA or DIPEA or | N,N-diisopropylethylamine or Hunig's base |
| iPr₂NEt | |
| DMA | dimethylacetamide |
| DMF | dimethyl formamide |
| DMAP | 4-dimethylaminopyridine |
| DMSO | dimethylsulfoxide |
| EtOAc or EA | ethyl acetate |
| EtOH | ethanol |
| EZ-Prep | CombiFlash ® EZ Prep System by Teledyne ISCO |
| HATU | hexafluorophosphate azabenzotriazole tetramethyl uronium |
| HBr | hydrobromic acid |
| HBPin | pinacolborane |
| HCl | hydrochloric acid |
| (HCHO)n | formaldehyde |
| HPLC | high performance liquid chromatography |
| h, hr, min, s | hour (h or hr), minute (min), second (s) |
| iPrOH | isopropyl alcohol |
| K₂CO₃ | potassium carbonate |
| KOAc | potassium acetate |
| KOH | potassium hydroxide |
| Lawesson's reagent | 2,4-Bis-(4-methoxyphenyl)-1,3-dithia-2,4-diphosphetane 2,4- |
| | disulfide |
| LC/MS, LCMS or LC-MS | liquid chromatographic mass spectroscopy |
| LDA | lithium diisopropylamide |
| M | molarity |
| MeI | iododmethane |
| MeOH | methanol |
| MOM-Br | Bromomethyl methyl ether |
| MS | mass spectroscopy |
| MsCl | methanesulfonyl chloride |
| Mg₂SO₄ | magnesium sulfate |
| N₂ | nitrogen gas |
| NaBH(OAc)₃ | sodium triacetoxyborohydride and sodium |
| | triacetoxyhydroborate |
| NaH | sodium hydride |
| NaHCO₃ | sodium bicarbonate |
| NaNO₂ | sodium nitrite |
| NaOH | sodium hydroxide |
| Na₂SO₄ | sodium sulfate |
| NH₃ | ammonia |
| NH₄HCO₃ | ammonium bicarbonate |
| NH₄OH | ammonium hydroxide |
| N₂H₄H₂O | hydrazine hydrate |
| NMP | N-methylpyrrolidone |
| NMR | nuclear magnetic resonance |
| Pd(OAc)₂ | palladium(II) acetate |
| Pd/C. ° | palladium on carbon |
| Pd₂(dba)₃ or Pd₂dba₃ | tris(dibenzylideneacetone)dipalladium(0) |
| Pd(dppf)Cl₂ | [1,1′-bis(diphenylphosphino)ferrocene] dichloropalladium(II) |
| PE | petroleum ether |
| PhMe | toluene |
| POBr₃ | phosphoryl bromide or phosphorus oxybromide |
| prep-HPLC | preparative high performance liquid chromatography |
| RT or R.T. or rt | room temperature |
| RuPhos | 2-Dicyclohexylphosphino-2′,6′-diisopropoxybiphenyl |
| sat. | saturated |
| SFC | supercritical fluid chromatography |
| SGC | solvating gas chromatography |
| SiO₂ | silicon dioxide or silica gel |
| TBAF | tetra-n-butylammonium fluoride |
| t-Bu | tert-butyl |
| TEA, NEt₃, Et₃N | triethylamine |
| TFA | trifluoroacetic acid |
| THF | tetrahydrofuran |
| TLC | thin layer chromatography |
| TosMIC, TsMIC | Toluenesulfonylmethyl isocyanide |
| UPLC | Ultra Performance Liquid Chromatography |
| Pd XPhos G3 | (2-dicyclohexylphosphino-2′,4′,6′-triisopropyl-1,1′-biphenyl)[2- |
| | (2′-amino-1,1′-biphenyl)]palladium(II) methanesulfonate |
| Pd XPhos G4 | methanesulfonato(2-dicyclohexylphosphino-2′,4′,6′-tri-i-propyl- |
| | 1,1′-biphenyl)(2′-methylamino-1,1′-biphenyl-2-yl)palladium(II) |

### Intermediate 1a: 1-Chloro-7-methylpyrrolo[1,2-d][1,2,4]triazin-4(3H)-one

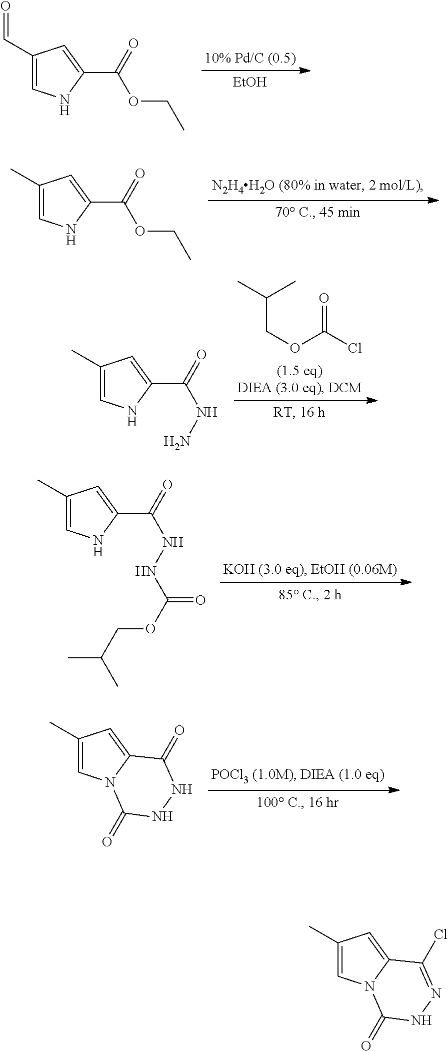

### Step 1. Ethyl 4-methyl-1H-pyrrole-2-carboxylate

A mixture of ethyl 4-formyl-1H-pyrrole-2-carboxylate (10.3 g, 61.6 mmol, 1.0 eq.) and Pd/C (10%, 2.0 g) in EtOH (150 mL) was stirred at rt overnight under hydrogen (balloon). The mixture was filtered. The filtrate was concentrated under reduced pressure. The crude colorless oil (8.50 g, 90% yield) was used in the next step without further purification. MS m/z 154.2 [M+H]⁺.

### Step 2. 4-Methyl-1H-pyrrole-2-carbohydrazide

A solution of ethyl 4-methyl-1H-pyrrole-2-carboxylate (8.50 g, 55.5 mmol) in N₂H₄H₂O (80%, 28 mL) was heated at 70° C. for 45 min. After cooling to rt, the mixture was filtered. The filter cake was washed with EtOH and dried in vacuo to give 4-methyl-1H-pyrrole-2-carbohydrazide as a white solid (7.5 g, 97% yield). MS m/z 140.2 [M+H]⁺; ¹H NMR (400 MHz, DMSO-d₆) δ 11.07 (s, 1H), 9.14 (s, 1H), 6.62 (s, 1H), 6.55 (s, 1H), 4.27 (s, 2H), 2.00 (s, 3H).

### Step 3. Isobutyl 2-(4-methyl-1H-pyrrole-2-carbonyl) hydrazine-1-carboxylate

To a suspension of 4-methyl-1H-pyrrole-2-carbohydrazide (7.5 g, 53.9 mmol, 1.0 eq.) and DIEA (20.9 g, 161 mmol, 3.0 eq.) in DCM (540 mL) was added isobutyl carbonochloridate (11.1 g, 80.9 mmol, 1.5 eq.) dropwise with ice-water cooling bath. The mixture was stirred at rt overnight. Upon completion, the reaction mixture was diluted with DCM, washed with water and brine. The organic phase was dried over Na₂SO₄, filtered, and concentrated under reduced pressure. The crude residue was purified by silica gel column chromatography eluting with 0-30% EtOAc in hexanes to afford isobutyl 2-(4-methyl-1H-pyrrole-2-carbonyl) hydrazine-1-carboxylate (12.0 g, 93%) as a white solid. MS m/z 240.2 [M+H]⁺.

### Step 4. 7-Methyl-2,3-dihydropyrrolo[1,2-d][1,2,4]triazine-1,4-dione

To a solution of isobutyl 2-(4-methyl-1H-pyrrole-2-carbonyl) hydrazine-1-carboxylate (12.0 g, 50.2 mmol, 1.0 eq.) in EtOH (840 mL) was added KOH (8.40 g, 151 mmol, 3.0 eq.). The mixture was stirred at 85° C. for 2 h. The reaction mixture was cooled to room temperature and filtered. The filter cake was washed with EtOH. The solid was dissolved with water, and adjusted to pH=4 with 1M HCl. The precipitate was filtered. The filter cake was washed with water and dried in vacuo to afford 7-methyl-2,3-dihydropyrrolo[1,2-d][1,2,4]triazine-1,4-dione (4.0 g, 48% yield) as a white solid. MS m/z 166.2 [M+H]⁺.

### Step 5. 1-Chloro-7-methylpyrrolo[1,2-d][1,2,4]triazin-4(3H)-one

To a solution of 7-methyl-2,3-dihydropyrrolo[1,2-d][1,2,4]triazine-1,4-dione (4.0 g, 24.2 mmol, 1.0 eq.) in POCl₃ (1.0 M, 25 mL) was added DIEA (3.12 g, 24.2 mmol, 1.0 eq.) dropwise with ice cooling bath. The mixture was stirred at 100° C. for 16 h, then cooled to room temperature and concentrated. The residue was carefully poured into ice water, adjusted pH=8 with sat NaHCO₃ aq. The mixture was extracted with DCM: MeOH (10:1, 100 mL×3), dried over Na₂SO₄, concentrated, purified by silica gel column chromatography eluting with 0-50% EtOAc in hexanes to afford 1-chloro-7-methylpyrrolo[1,2-d][1,2,4]triazin-4(3H)-one (2.0 g, 45% yield) as a white solid. MS m/z 184.3 [M+H]⁺; ¹H NMR (400 MHz, DMSO-d₆) δ 12.43 (s, 1H), 7.66 (s, 1H), 6.75 (s, 1H), 2.23 (s, 3H).

### Intermediate 1b: 8-Fluoro-2,3-dihydropyrrolo[1,2-d][1,2,4]triazine-1,4-dione

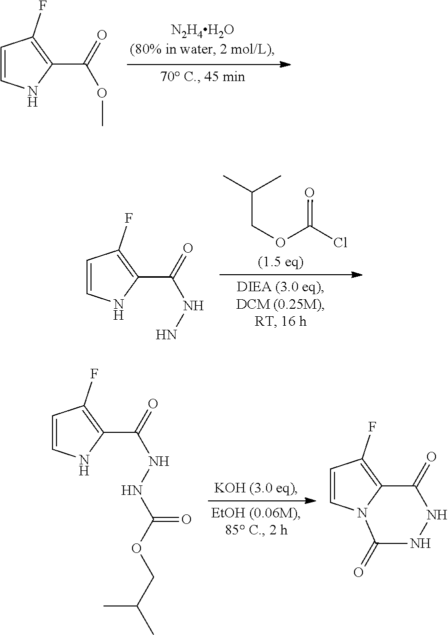

### Step 1. 3-Fluoro-1H-pyrrole-2-carbohydrazide

A mixture of N₂H₄·H₂O (80% in water, 18 mL) and methyl 3-fluoro-1H-pyrrole-2-carboxylate (3.14 g, 20.0 mmol) was stirred at 70° C. for 45 minutes. After cooling to rt, the precipitate was filtered, the filter cake was washed with water and dried under vacuum to obtain 3-fluoro-1H-pyrrole-2-carbohydrazide (2.30 g, 73%) as a white solid. MS m/z 144.1 [M+H]⁺, ¹H NMR (400 MHz, DMSO-d₆) δ 11.31 (s, 1H), 8.55 (s, 1H), 6.74 (dd, J=4.6, 3.0 Hz, 1H), 5.97 (d, J=3.0 Hz, 1H), 4.39 (s, 2H).

### Step 2. Isobutyl 2-(3-fluoro-1H-pyrrole-2-carbonyl)hydrazine-1-carboxylate

To a solution of 3-fluoro-1H-pyrrole-2-carbohydrazide (2.15 g, 15.0 mmol, 1.0 eq.) in DCM (60 mL) was added DIPEA (5.80 g, 45.0 mmol, 3.0 eq.). Isobutyl carbonochloridate (3.00 g, 22.5 mmol, 1.5 eq.) was added to the mixture slowly. The mixture was stirred at rt for 16 h. After concentration, the mixture was diluted with EtOAc (200 mL). The organic layer was washed with water and brine, then dried over Na₂SO₄, filtered and concentrated under reduced pressure. The crude was purified by silica gel column chromatography eluting with 0-50% EtOAc in hexanes to afford isobutyl 2-(3-fluoro-1H-pyrrole-2-carbonyl)hydrazine-1-carboxylate (2.44 g, 66.7% yield) as yellow solid. MS m/z 244.1 [M+H]⁺; ¹H NMR (400 MHz, DMSO-d₆) δ 11.46 (s, 1H), 9.18 (s, 1H), 9.09 (s, 1H), 6.82 (d, J=4.4 Hz, 1H), 6.01 (d, J=2.5 Hz, 1H), 3.82 (d, J=6.6 Hz, 2H), 2.02-1.67 (m, 1H), 0.92 (d, J=6.7 Hz, 6H).

### Step 3. 8-Fluoro-2,3-dihydropyrrolo[1,2-d][1,2,4]triazine-1,4-dione

To a solution of isobutyl 2-(3-fluoro-1H-pyrrole-2-carbonyl)hydrazine-1-carboxylate(2.44 g, 10.0 mmol, 1.0 eq.) in EtOH (60 mL) was added KOH (1.68 g, 30 mmol, 3 eq.). The mixture was stirred at 85° C. for 2 h. After cooling to rt, the mixture was concentrated. The residue was diluted with water (50 mL) and adjusted to pH to 6-7 with 1M HCl. The precipitates were filtered, the filter cake was washed with water and dried under vacuum to obtain 8-fluoro-2,3-dihydropyrrolo[1,2-d][1,2,4]triazine-1,4-dione (1.16 g, 68.7% yield) as brown solid. MS m/z 168.0 [M−H]⁻; ¹H NMR (400 MHz, DMSO-d₆) δ 11.52 (s, 2H), 7.57 (t, J=4.1 Hz, 1H), 6.68 (d, J=3.4 Hz, 1H).

### Intermediate 1c: 1-chloro-4-(methylthio)pyrrolo[1,2-d][1,2,4]triazine

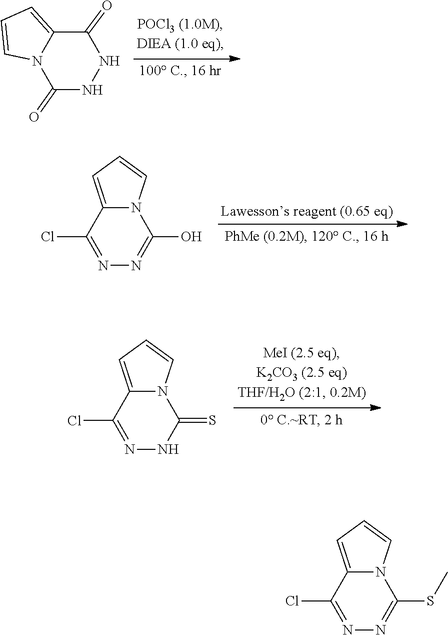

### Step 1. 1-Chloropyrrolo[1,2-d][1,2,4]triazin-4-ol

To a stirred solution of 2,3-dihydropyrrolo[1,2-d][1,2,4]triazine-1,4-dione (prepared as the procedure of Intermediate 1b, 2 g, 13.2 mmol) in POCl₃ (60 mL) was added DIEA (1.84 g, 14.2 mmol). The reaction mixture was heated at 100° C. for 16 h. After cooling to rt, the mixture was concentrated under reduced pressure to give a residue. The residue was diluted with cold saturated sodium bicarbonate solution and extracted with dichloromethane (3×50 mL). The combined organic extracts were dried over sodium sulfate and evaporated under reduced pressure. The crude was further triturated with DCM to give the title compound 1-chloropyrrolo[1,2-d][1,2,4]triazin-4-ol (0.867 g, 38.6% yield) as an off-white solid, which was used to next step without further purification. MS m/z 169.6 [M+H]⁺; ¹H NMR (400 MHz, DMSO-d₆) δ 12.54 (s, 1H), 7.88-7.87 (m, 1H), 6.92-6.89 (m, 2H).

### Step 2. 1-Chloropyrrolo[1,2-d][1,2,4]triazine-4(3H)-thione

To a solution of 1-chloropyrrolo[1,2-d][1,2,4]triazin-4-ol (30.0 g, 177 mmol) in toluene (1200 mL) was added Lawesson's Reagent (46.2 g, 114 mmol). The reaction was stirred at 120° C. for 16 h. The mixture was diluted with H₂O (1.5 L), acidified with aqueous HCl (1 M) till pH=3 and extracted with EA. The EA layer was wash with sat. NaHCO₃ aqueous solution (1 L) and brine (1 L), then dried over anhydrous Na₂SO₄ and concentrated to give crude 1-chloro-3H-pyrrolo[1,2-d][1,2,4]triazine-4-thione (crude 35.0 g, overweight), which was used in the next step without further purification. MS m/z 184.1 [M−H]⁻.

### Step 3. 1-Chloro-4-(methylthio)pyrrolo[1,2-d][1,2,4]triazine

To a solution of 1-chloro-3H-pyrrolo[1,2-d][1,2,4]triazine-4-thione (crude 35.0 g, 189 mmol) in THE (600 mL) and water (300 mL) was added K₂CO₃ (65.2 g, 472 mmol), followed by iodomethane (68.6 g, 483 mmol) at 0° C. The reaction mixture was stirred for 2 h at room temperature, then diluted with water and extracted with EA (3×100 mL). The combined organic layers were washed with water, brine, dried over anhydrous Na₂SO₄ and concentrated. The residue was purified by column chromatography (SiO₂, Petroleum ether/Ethyl acetate=50/1 to 20/1) to afford title product 1-chloro-4-methylsulfanyl-pyrrolo[1,2-d][1,2,4]triazine (21.8 g, 57.9% yield over two steps) as a white solid. MS m/z 200.0 [M+H]f, 1H NMR (400 MHz, DMSO-d₆) δ: 7.83 (dd, J=2.8 Hz, 1.2 Hz, 1H), 7.16 (dd, J=4.0 Hz, 2.8 Hz, 1H), 7.11 (dd, J=4.0 Hz, 1.2 Hz, 1H), 2.81 (s, 3H).

The intermediates below were prepared according to the procedure of Intermediate 1c by substituting the appropriate starting materials, reagents and reaction conditions.

| Structure | Spectral Data |
|---|---|
| 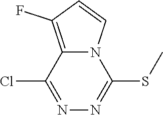 | From Intermediate 1b; ¹H NMR (400 MHz, DMSO-d₆) δ 7.65 (s, 1H), 6.94 (s, 1H), 2.79 (s, 1H), 2.33 (s, 1H). |
| 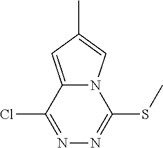 | From Intermediate la; ¹H NMR (400 MHz, DMSO-d₆) δ 7.73-7.71 (m, 1H), 7.14 (d, J = 3.2 Hz, 1H), 2.79 (s, 3H). |

### Intermediate 1d: 1-Bromo-4-(methylthio)pyrrolo[1,2-d][1,2,4]triazine

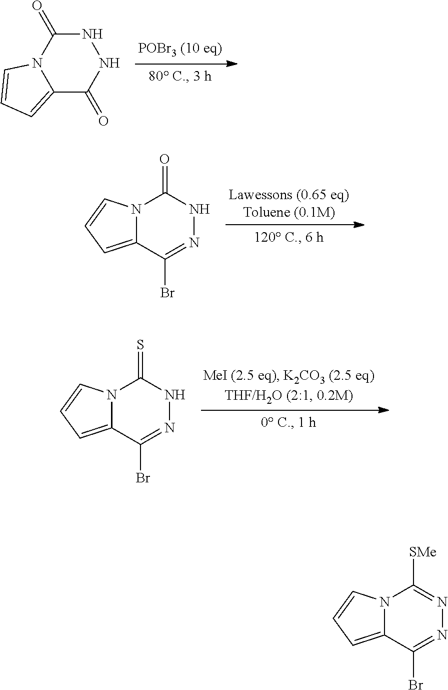 [SMILES: O=c1[nH][nH]c(=O)n2cccc12 ; O=c1[nH]nc(Br)c2cccn12 ; S=c1[nH]nc(Br)c2cccn12 ; CSc1nnc(Br)c2cccn12]

### Step 1. 1-Bromopyrrolo[1,2-d][1,2,4]triazin-4(3H)-one

A mixture of 2,3-dihydropyrrolo[1,2-d][1,2,4]triazine-1,4-dione (30.0 g, 0.199 mol, 1.0 eq.) and POBr₃ (570 g, 1.99 mol, 10 eq.) was heated at 80° C. for 3 hours. The hot reaction mixture was slowly poured into a stirring mixture of ice and aqueous sat. NaHCO₃. The mixture was neutralized with solid NaHCO₃ to pH˜7 and extracted with a mixture of MeOH/DCM (1 L×2, 1:10). The combined organic phase was dried over Na₂SO₄, filtered, and concentrated under reduced pressure. The crude residue was triturated with DCM, filtered and washed with DCM. The solid was dried under vacuum to afford 1-bromopyrrolo[1,2-d][1,2,4]triazin-4(3H)-one (21.9 g, 51.8% yield) as a pink solid. MS m/z 214.0, 215.9 [M+H]⁺.

### Step 2. 1-Bromopyrrolo[1,2-d][1,2,4]triazine-4(3H)-thione

To a solution of 1-bromopyrrolo[1,2-d][1,2,4]triazin-4(3H)-one (10.0 g, 46.7 mmol, 1.0 eq.) in DMSO (472 mL) was added Lawesson's Reagent (12.4 g, 30.7 mmol, 0.65 eq.). The reaction mixture was heated at 120° C. for 6 hours. The reaction mixture was cooled to room temperature, diluted with aqueous saturated NaHCO₃ and extracted with EtOAc (300 mL×3). The organic layer was dried over Na₂SO₄, filtered and concentrated under reduced pressure. The crude was purified by silica gel column chromatography eluting with 0-30% EtOAc in hexanes to afford 1-bromopyrrolo[1,2-d][1,2,4]triazine-4(3H)-thione as a white solid (1.56 g, 14.5% yield). MS m/z 228.0, 230.0 [M−H]; H NMR (400 Hz, DMSO-d₆) δ 14.15 (s, 1H), 8.23 (dd, J=2.9, 1.4 Hz, 1H), 7.10 (dd, J=3.7, 3.1 Hz, 1H), 7.06 (dd, J=3.9, 1.4 Hz, 1H).

### Step 3. 1-Bromo-4-(methylthio)pyrrolo[1,2-d][1,2,4]triazine

To a mixture of 1-bromopyrrolo[1,2-d][1,2,4]triazine-4(3H)-thione (2.00 g, 8.69 mmol, 1.0 eq.) and K₂CO₃ (3.00 g, 21.7 mmol, 2.5 eq.) in THE (43 mL) and water (21 mL) was added iodomethane (1.35 mL, 2.28 g/mL, 21.7 mmol, 2.5 eq.) dropwise at 0° C. The reaction was stirred at 0° C. for 1 hour. The reaction mixture was diluted with water (50 mL) and extracted with EtOAc (50 mL×3). The organic layer was dried over Na₂SO₄, filtered and concentrated under reduced pressure. The crude product was purified by silica gel column chromatography eluting with 0-30% EtOAc in hexanes to afford 1-bromo-4-(methylthio)pyrrolo[1,2-d][1,2,4]triazine (1.40 g, 66.1% yield) as a white solid. MS m/z 243.9, 246.0 [M+H]⁺; ¹H NMR (400 Hz, DMSO-d₆) δ 7.83 (dd, J=2.8, 1.0 Hz, 1H), 7.15 (dd, J=4.0, 2.8 Hz, 1H), 7.03 (dd, J=4.0, 1.0 Hz, 1H), 2.79 (s, 3H).

### Intermediate 1e: 1-Bromo-4-(methylthio)imidazo[1,5-d][1,2,4]triazine

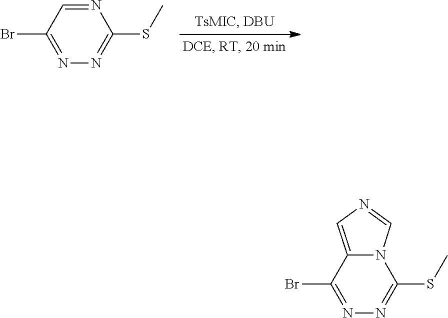

To a solution of TsMIC (5.21 g, 26.69 mmol, 1.1 eq.) and DBU (4.43 mg, 29.12 mmol, 1.2 eq.) in DCE (50 mL) was added 6-Bromo-3-(methylthio)-1,2,4-triazine (5 g, 24.26 mmol, 1.0 eq.) at rt. The reaction mixture was stirred for 20 min, then diluted with water (50 mL) and extracted with EtOAc (50 mL×3). The combined organic layers were washed with brine, dried over anhydrous Na₂SO₄ and concentrated. The residue was purified by flash chromatography on silica gel (PE:EA=15:1) to afford 1-bromo-4-(methylthio)imidazo[1,5-d][1,2,4]triazine (3.71 g, 62.38% yield) as yellow solid. MS m/z 244.9, 246.9 [M+1]*; 1H NMR (400 MHz, DMSO-d₆) δ 8.84 (s, 1H), 7.93 (s, 1H), 2.80 (s, 3H).

### Intermediate If: 7-Bromo-2-methylpyrazolo[1,5-d][1,2,4]triazin-4-ol

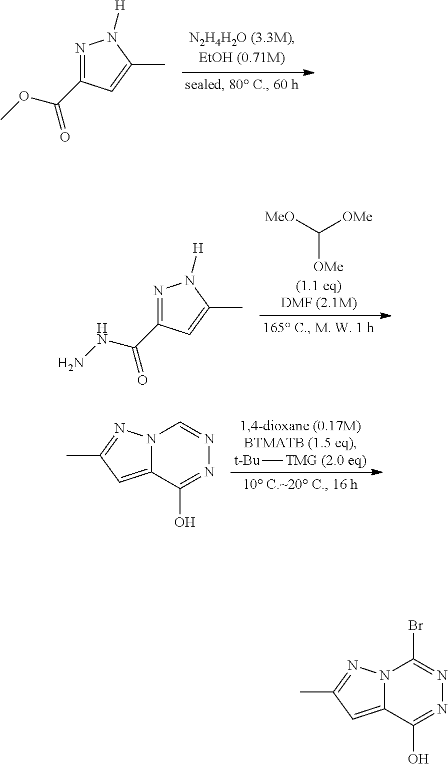

### Step 1. 5-Methyl-1H-pyrazole-3-carbohydrazide

Hydrazine hydrate (54 mL, 1.032 g/mL, 1.11 mol, 6.2 eq.) was added to a solution of methyl 5-methyl-1H-pyrazole-3-carboxylate (25.0 g, 0.178 mol, 1.0 eq.) in EtOH (250 mL). The mixture was sealed and heated at 80° C. for 60 h, then cooled to room temperature. The solvent was evaporated in vacuo. The crude product was treated with a 1/1 mixture of water/MeOH, filtered and the filter cake was washed with water. The solid was dried under vacuum to afford 5-methyl-1H-pyrazole-3-carbohydrazide (21.7 g, 87.0% yield) as a white solid. MS m/z 141.1 [M+H]⁺.

### Step 2. 2-Methylpyrazolo[1,5-d][1,2,4]triazin-4-ol

A mixture of 5-methyl-1H-pyrazole-3-carbohydrazide (3.00 g, 21.4 mmol, 1.0 eq.) and trimethoxymethane (2.57 mL, 0.970 g/mL, 23.5 mmol, 1.1 eq.) in DMF (10 mL) was sealed in a tube. The reaction mixture was stirred at 165° C. for 1 hour under microwave then cooled to room temperature. The precipitate was collected by filtration, washed with EtOH and dried under vacuum to afford 2-methylpyrazolo[1,5-d][1,2,4]triazin-4-ol (1.70 g, 52.9% yield) as a white solid. MS m/z 149.2 [M−H]⁻.

### Step 3. 7-Bromo-2-methylpyrazolo[1,5-d][1,2,4]triazin-4-ol

Benzyltrimethylammonium tribromide (26.5 g, 68.0 mmol, 1.5 eq.) was added in portions to a stirred solution of 2-methylpyrazolo[1,5-d][1,2,4]triazin-4-ol (6.80 g, 45.3 mmol, 1.0 eq.) and 2-(tert-butyl)-1,1,3,3-tetramethylguanidine (18.3 mL, 0.85 g/mL, 90.6 mmol, 2.0 eq.) in 1,4-dioxane (290 mL) under N₂ at 10° C. Once the addition was completed the mixture was warmed to 20° C. and stirred at this temperature for 16 hours. Then the mixture was quenched with a mixture of aqueous sat. Na₂S₂O₃ and aqueous sat. NaHCO₃ and then extracted with EtOAc (150 mL×3). The combined organic layers were dried over Na₂SO₄, filtered and concentrated. The crude product was triturated with EtOAc, filtered and washed with EtOAc. The solid was dried under vacuum to afford 7-bromo-2-methylpyrazolo[1,5-d][1,2,4]triazin-4-ol (7.60 g, 73.6% yield) as an off-white solid. MS m/z 229.0, 230.9 [M+H]⁺; H NMR (400 Hz, DMSO-d₆) δ 12.60 (s, 1H), 7.15 (s, 1H), 2.42 (s, 3H).

### Intermediate 2a: 2-(4-methoxy-2-(2,2,2-trifluoroethyl)phenyl)-4,4,5,5-tetramethyl-1,3,2-dioxaborolane

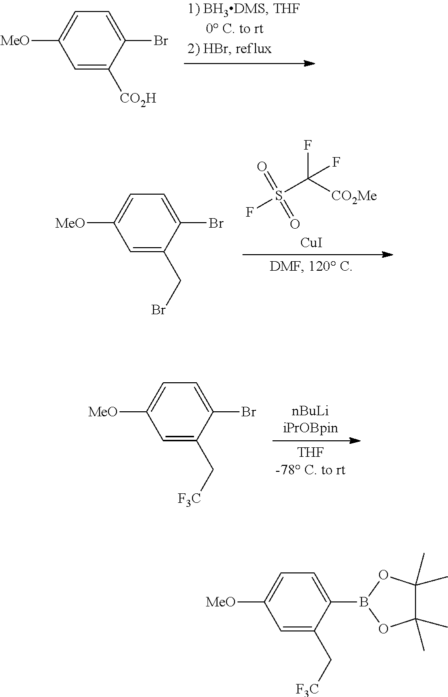

### Step 1: (2-bromo-5-methoxy-phenyl)methanol

To an ice-cooled solution of 2-bromo-5-methoxy-benzoic acid (9.95 g, 43.1 mmol) in THE (86 mL) was added borane-dimethylsulfide complex (6.01 mL, 64.6 mmol). The resulting mixture was warmed to rt and stirred for 16 hours. The reaction mixture was quenched with methanol and stirred for 5 min. The reaction was concentrated, and the resulting yellow oil was dissolved in EtOAc (150 mL) and washed with 10% Na₂CO₃ (30 mL). The aqueous phase was extracted with EtOAc. The combined organic extracts were washed with brine (50 mL), dried (MgSO₄) and concentrated to give a colorless oil, which was applied to the next step without further purification.

### Step 2: 1-bromo-2-(bromomethyl)-4-methoxy-benzene

The crude [2-bromo-5-(trifluoromethyl)phenyl]methanol in conc. HBr (30 mL) was heated to reflux for 2 hours. The reaction was cooled to rt and extracted with DCM. The combined organic extracts were washed with sat. NaHCO₃, brine, dried (Na₂SO₄), filtered, and concentrated to give a white solid. Purification by chromatography on SiO₂ (EtOAc:hexanes, 10 to 30%) gave a white solid (10.99 g, 91% 2 steps). ¹H NMR (400 MHz, CDCl₃) δ 7.46 (d, J=8.9 Hz, 1H), 7.00 (d, J=3.0 Hz, 1H), 6.75 (dd, J=8.9 Hz, 3.0 Hz, 1H), 4.57 (s, 2H), 3.81 (s, 3H).

### Step 3: 1-bromo-4-methoxy-2-(2,2,2-trifluoroethyl)benzene

To a solution of 1-bromo-2-(bromomethyl)-4-methoxy-benzene (5.62 g, 20.1 mmol) in DMF (45 ml) was added CuI (9.56 g, 50.2 mmol) and the solution was sparged with Ar. To this solution was added difluoro-fluorosulfonyl-acetic acid methyl ester (6.4 mL, 50.2 mmol), and the resulting reaction mixture was heated at 120° C. for 4 h. The reaction mixture was cooled to 0° C., diluted with EtOAc (200 ml) and stirred for 10 min. Conc. ammonium hydroxide (30 mL) was added dropwise, and the mixture was stirred at 0° C. for 15 min before warming to rt. EtOAc (200 ml) and water (100 mL) were added and the layers were separated. The aqueous layer was extracted with EtOAc (2×100 mL). The combined organic layers were washed with water (100 mL) and brine (100 ml), dried (MgSO₄), filtered, and concentrated. Purification by chromatography on SiO₂ (EtOAc:hexanes, 0 to 10%) gave a light yellow oil (4.13 g, 77%). ¹H NMR (400 MHz, CDCl₃) δ 7.50 (d, J=8.9 Hz, 1H), 6.93 (d, J=2.8 Hz, 1H), 6.78 (dd, J=8.8 Hz, 3.1 Hz, 1H), 3.81 (s, 3H), 3.60 (q, J=10.5 Hz, 2H).

### Intermediate 2b: 4,4,5,5-Tetramethyl-2-(2-(2,2,2-trifluoroethyl)-4-(trifluoromethyl)phenyl)-1,3,2-dioxaborolane

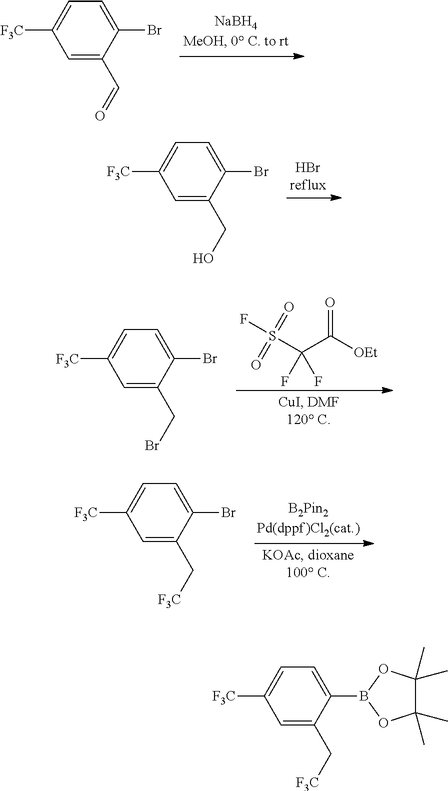

### Step 1. (2-Bromo-5-(trifluoromethyl)phenyl)methanol

To a solution of 2-bromo-5-(trifluoromethyl)benzaldehyde (30.0 g, 118.6 mmol, 1.0 eq.) in MeOH (300 mL) was added NaBH₄ (13.5 g, 3 eq.) slowly at 0° C. The mixture was stirred for 16 h at room temperature. The reaction mixture was diluted with water (100 mL) and extracted with EtOAc (250 mL×3). The combined organic layers were washed with brine, dried over anhydrous Na₂SO₄ and concentrated. The filtrate was concentrated in vacuo to afford (2-bromo-5-(trifluoromethyl)phenyl)methanol (27 g, 78.4% Yield) as white solid. The crude product used directly without further purification. ¹H NMR (400 MHz, CDCl₃) δ 7.81 (s, 1H), 7.67 (d, J=8.3 Hz, 1H), 7.44-7.39 (m, 1H), 4.81 (s, 2H).

### Step 2. 1-Bromo-2-(bromomethyl)-4-(trifluoromethyl)benzene

2-Bromo-5-(trifluoromethyl)phenyl)methanol (26.0 g, 102.0 mmol, 1.0 eq.) was added to HBr aqueous solution (156 mL). The reaction mixture was stirred for 2 h at 120° C. under N₂ atmosphere. The reaction mixture was diluted with NaHCO₃ (100 mL) and extracted with DCM (250 mL×3). The combined organic layers were washed with brine, dried over anhydrous Na₂SO₄ and concentrated. The residue was purified by flash chromatography on silica gel (PE: EA=0%˜10%) to afford 1-bromo-2-(bromomethyl)-4-(trifluoromethyl)benzene (25 g, 76.9% yield) as colorless oil. ¹H NMR (400 MHz, DMSO-d₆) δ 8.08 (d, J=2.0 Hz, 1H), 7.92 (d, J=8.4 Hz, 1H), 7.65 (dd, J=8.4, 2.1 Hz, 1H), 4.83 (s, 2H).

### Step 3. 1-Bromo-2-(2,2,2-trifluoroethyl)-4-(trifluoromethyl)benzene

To a solution of 1-bromo-2-(bromomethyl)-4-(trifluoromethyl)benzene (40.5 g, 127.4 mmol, 1.0 eq.) in DMF (280 mL) was added CuI (60.7 g, 2.5 eq.) and ethyl 2,2-difluoro-2-(fluorosulfonyl)acetate (65.7 g, 2.5 eq.) under N₂ at rt. The reaction mixture was stirred overnight at 120° C. under N₂ atmosphere. The reaction mixture was cooled to 0° C., diluted with EtOAc (1200 mL) and stirred for 10 minutes at 0° C. A solution of ammonium hydroxide (240 mL) was added dropwise. The mixture was stirred as it warmed from 0° C. to room temperature over 20 minutes. The reaction mixture was diluted with water (500 mL) and extracted with EtOAc (550 mL×3). The combined organic layers were washed with brine, dried over anhydrous Na₂SO₄ and concentrated. The residue was purified by flash chromatography on silica gel (PE:EA=0%˜10%) to afford 1-bromo-2-(2,2,2-trifluoroethyl)-4-(trifluoromethyl)benzene (16.8 g, 17.9% Yield) as yellow oil. ¹HNMR (400 MHz, DMSO-d₆) δ 7.95 (dd, J=15.1, 8.2 Hz, 2H), 7.70 (dd, J=8.4, 2.1 Hz, 1H), 3.97 (q, J=11.0 Hz, 2H).

### Step 4. 4,4,5,5-Tetramethyl-2-(2-(2,2,2-trifluoroethyl)-4-(trifluoromethyl)phenyl)-1,3,2-dioxaborolane

To a solution of 1-bromo-2-(2,2,2-trifluoroethyl)-4-(trifluoromethyl)benzene (16.8 g, 54.7 mmol, 1.0 eq.) in 1,4-dioxane (160 mL) was added 4,4,4′,4′,5,5,5′,5′-octamethyl-2,2′-bi(1,3,2-dioxaborolane) (27.8 g, 2 eq.), Pd(dppf)Cl₂ (4.0 g, 0.1 eq.) and KOAc (16.1 g, 3 eq.) under N₂ atmosphere at rt. The reaction mixture was stirred for 12 h at 100° C. under N₂. The reaction mixture was diluted with water (90 mL) and extracted with EtOAc (150 mL×3). The combined organic layers were washed with brine, dried over anhydrous Na₂SO₄ and concentrated. The residue was purified by flash chromatography on silica gel (PE:EA=0%˜10%) to afford 4,4,5,5-tetramethyl-2-(2-(2,2,2-trifluoroethyl)-4-(trifluoromethyl)phenyl)-1,3,2-dioxaborolane (10.5 g, 45.8% Yield) as yellow oil. ¹H NMR (400 MHz, DMSO-d₆) δ 7.95 (d, J=7.8 Hz, 1H), 7.79-7.73 (m, 2H), 4.08 (q, J=11.2 Hz, 2H), 1.32 (s, 12H).

The intermediates below were prepared according to the procedure of Intermediate 2a and 2b by substituting the appropriate starting materials, reagents and reaction conditions.

| Structure | Spectral Data |
|---|---|
| 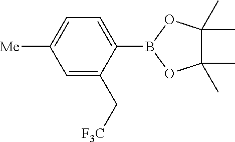 [SMILES: Cc1ccc(B2OC(C)(C)C(C)(C)O2)c(CC(F)(F)F)c1] | ¹H NMR (400 MHz, CDCl₃) δ 7.76 (d, J = 7.5 Hz, 1H), 7.15 (d, J = 7.8 Hz, 1H), 7.12 (s, 1H), 3.81 (q, J = 11.0 Hz, 2H), 2.37 (s, 3H), 1.34 (s, 12H). |
| 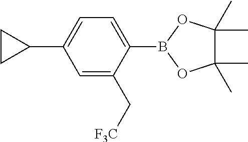 [SMILES: CC1(C)OB(c2ccc(C3CC3)cc2CC(F)(F)F)OC1(C)C] | ¹H NMR (500 MHz, CDCl₃) δ 7.75 (d, J = 8.2 Hz, 1H), 7.02-6.98 (m, 2H), 3.81 (q, J = 10.9 Hz, 2H), 1.94-1.84 (m, 1H), 1.33 (s, 12H), 1.02-0.95 (m, 2H), 0.76-0.71 (m, 2H). |

### Intermediate 2c: 2-[2-(Difluoromethyl)-4-(trifluoromethyl)phenyl]-4,4,5,5-tetramethyl-1,3,2-dioxaborolane

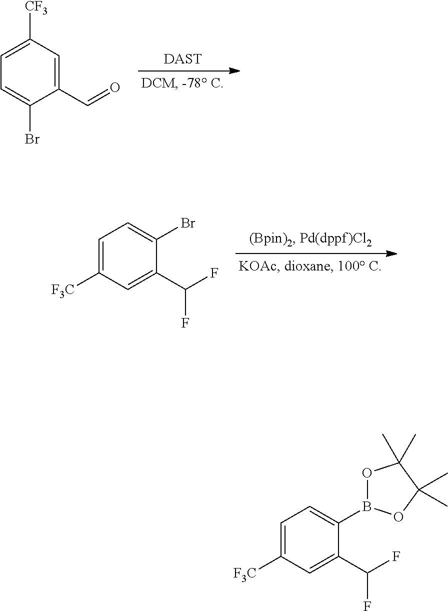

### Step 1. 1-Bromo-2-(difluoromethyl)-4-(trifluoromethyl)benzene

DAST (10 eq., 39.5 mmol) was added dropwise to a −78° C. solution of 2-bromo-5-(trifluoromethyl)benzaldehyde (1 g, 3.9 mmol) in DCM (5 mL, 78.0 mmol). The reaction was stirred for 15 min and then allowed to warm to room temperature. After 4 hours, TLC showed incomplete conversion, so the mixture was cooled again to −78° C. and additional DAST (1 eq., 3.9 mmol) was added. The mixture was allowed to warm to rt and stirred overnight, then poured in ice and dilute NH₄OH and extracted 2x with DCM. The combined organic extracts were washed with brine and dried over sodium sulfate. Solvent was evaporated in vacuum to give a residue which was purified with a short plug of silica to give the crude product 1-bromo-2-(difluoromethyl)-4-(trifluoromethyl)benzene (350 mg, 1.2 mmol, 32.2% Yield) as colorless oil.

### Step 2. 2-[2-(Difluoromethyl)-4-(trifluoromethyl)phenyl]-4,4,5,5-tetramethyl-1,3,2-dioxaborolane

To a solution of 1-bromo-2-(difluoromethyl)-4-(trifluoromethyl)benzene (100 mg, 0.36 mmol) in 1,4-dioxane (2 mL) was added bis(pinacolato)diboron (1.5 eq., 0.5 mmol), potassium acetate (2 eq., 0.72 mmol) and [1,1′-bis(diphenylphosphino)ferrocene]dichloropalladium(ii) (0.15 eq., 0.05 mmol). The mixture was stirred for 16 h at 100° C. for 16 hours under N₂ atmosphere, monitored by TLC and LCMS. After the reaction, the solvent was removed under vacuum and the residue was purified by column chromatography (10-15% EA in PE) to afford 2-[2-(difluoromethyl)-4-(trifluoromethyl)phenyl]-4,4,5,5-tetramethyl-1,3,2-dioxaborolane (30 mg, 0.09 mmol, 25.6% Yield) as colorless oil. ¹H NMR (400 MHz, DMSO-d₆) δ 8.00 (d, J=7.6 Hz, 1H), 7.94 (d, J=8.4 Hz, 1H), 7.92 (s, 1H), 7.38 (t, J=55.6 Hz, 1H), 1.34 (s, 12H).

### Intermediate 2d: 2-(4-Chloro-2-(difluoromethyl)phenyl)-4,4,5,5-tetramethyl-1,3,2-dioxaborolane

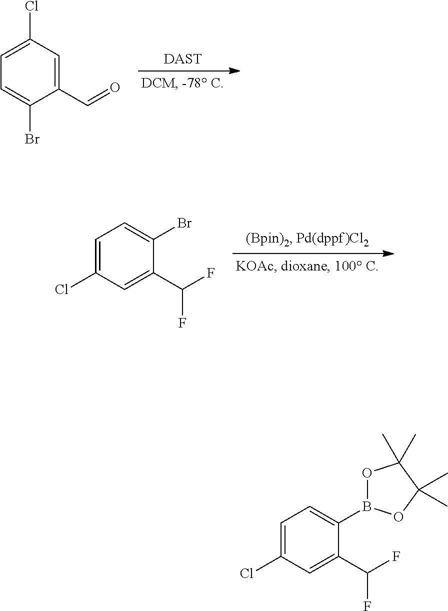

The title compound was prepared in analogous manner according to the procedure of Intermediate 2c, using 2-bromo-5-chlorobenzaldehyde in place of 2-bromo-5-(trifluoromethyl)benzaldehyde in step 1. ¹H NMR (400 MHz, DMSO-d₆) δ 7.79 (d, J=2.0 Hz, 1H), 7.68-7.63 (m, 2H), 7.32 (t, J=55.6 Hz, 1H), 1.32 (s, 12H).

### Intermediate 2e: 2-(2-(Difluoromethoxy)-4-(trifluoromethyl)phenyl)-4,4,5,5-tetramethyl-1,3,2-dioxaborolane

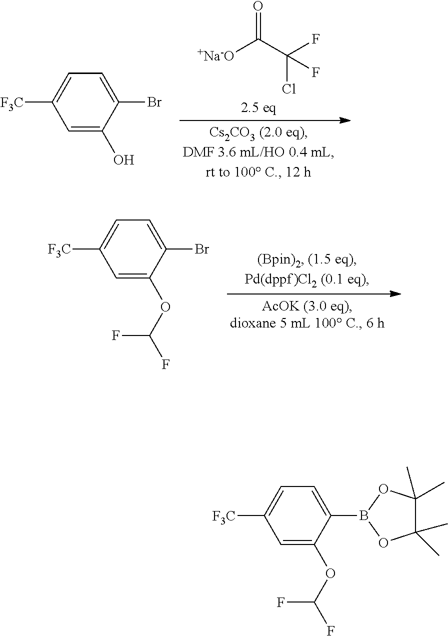

### Step 1. 1-Bromo-2-(difluoromethoxy)-4(trifluoromethyl)benzene

A mixture of 2-bromo-5-(trifluoromethyl)phenol (5.00 g, 20.7 mmol, 1.0 eq.), sodium 2-chloro-2,2-difluoroacetate (7.07 g, 51.9 mmol, 2.50 eq.) and cesium carbonate (11.5 g, 41.5 mmol, 2.0 eq.) in water (20 mL) and N,N-dimethylformamide (80 mL) was heated at 100° C. for 12 hours. Upon completion, the reaction mixture was cooled to room temperature and diluted with EtOAc. The reaction mixture was washed with water and brine. The organic phase was dried over Na₂SO₄, filtered, and concentrated under reduced pressure. The crude residue was purified by silica gel column chromatography eluting with 0-50% EtOAc in hexanes to afford 1-bromo-2-difluoromethoxy)-4(trifluoromethyl)benzene (2.8 g, 47% yield) as a yellow solid. ¹H NMR (400 MHz, DMSO-d₆) δ 8.01 (d, J=8.4 Hz, 1H), 7.69 (s, 1H), 7.46 (t, J=72.8 Hz, 1H), 7.5 (d, J=8.4 Hz, 1H).

### Step 2. 2-(2-(Difluoromethoxy)-4-(trifluoromethyl)phenyl)-4,4,5,5-tetramethyl-1,3,2-dioxaborolane

To a solution of 1-bromo-2-difluoromethoxy)-4(trifluoromethyl)benzene (2.80 g, 9.62 mmol) in 1,4-dioxane (30 mL) was added bis(pinacolato)diboron (36.6 g, 14.4 mmol, 1.50 eq.), [1,1′-bis(diphenylphosphino)ferrocene]dichloropalldium(II) (704 mg, 0.96 mmol, 0.1eq.), potassium acetate (2.82 g, 28.9 mmol, 3.0 eq.). The reaction mixture was heated at 100° C. for 16 hours under nitrogen. The reaction mixture was cooled to room temperature and filtered. The filtrate was diluted EA, washed with water and brine. The organic phase was dried over Na₂SO₄, filtered and concentrated under reduced pressure. The crude was purified by silica gel column chromatography eluting with hexane to afford 2-(2-(difluoromethoxy)-(trifluoromethyl)phenyl)-4,4,5,5-tetramethyl-1,3,2-dioxaborolane as a white solid (1.70 g, 52% yield). ¹H NMR (400 MHz, DMSO-d₆) δ 7.87 (d, J=7.6 Hz, 1H), 7.66 (d, J=7.6 Hz, 1H), 7.50 (s, 1H), 7.24 (t, J=74.0 Hz, 1H), 1.31 (s, 12H).

### Intermediate 2f: 2-(2,4-Bis(difluoromethoxy)phenyl)-4,4,5,5-tetramethyl-1,3,2-dioxaborolane

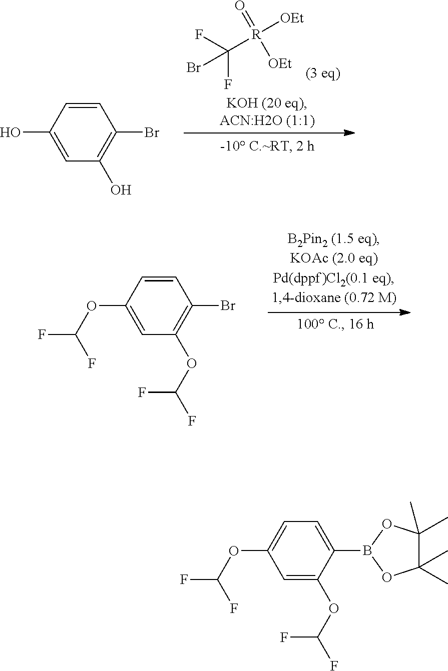

### Step 1. 1-Bromo-2,4-bis(difluoromethoxy)benzene

To a solution of 4-bromobenzene-1,3-diol (10.0 g, 53.4 mmol, 1.0 eq.) in ACN/H₂O (1:1, 100 mL) was slowly added KOH (60.0 g, 1.07 mol, 20 eq.) at 0° C. The mixture was stirred for 10 min and cooled to −10° C. Diethyl (bromodifluoromethyl) phosphonate (42.70 g, 160.2 mmol, 3.0 eq.) was slowly added and the mixture was stirred for 10 min and warmed to room temperature and stirred for 2 h. The reaction mixture was diluted with water (100 mL) and extracted with EtOAc (100 mL×3). The combined organic layers were washed with brine, dried over anhydrous Na₂SO₄ and concentrated. The residue was purified by flash chromatography on silica gel (PE:EA=0%˜5%) to give 1-bromo-2,4-bis(difluoromethoxy)benzene (3.30 g, 21.3% Yield) as yellow oil. ¹H NMR (400 MHz, DMSO-d₆) δ 7.84 (d, J=8.8 Hz, 1H), 7.38 (t, J=72.0 Hz, 1H), 7.35 (t, J=76.0 Hz, 1H), 7.26 (d, J=2.8 Hz, 1H), 7.11 (dd, J=8.8, 2.8 Hz, 1H).

### Step 2. 2-(2,4-Bis(difluoromethoxy)phenyl)-4,4,5,5-tetramethyl-1,3,2-dioxaborolane

To a solution of 1-bromo-2,4-bis(difluoromethoxy)benzene (3.30 g, 11.4 mmol, 1.0 eq.) in 1,4-dioxane was added KOAc (2.23 g, 22.8 mmol, 2.0 eq.), Pd(dppf)C₁₂ (0.830 g, 1.10 mmol, 0.1 eq.) and bis(pinacolato)diboron (4.34 g, 17.1 mmol, 1.5 eq.). The reaction mixture was stirred for 16 h at 100° C. under N₂. The mixture was filtered and concentrated. The residue was purified by flash chromatography on silica gel (PE:EA=0%˜10%) to obtain 2-(2,4-bis(difluoromethoxy) phenyl)-4,4,5,5-tetramethyl-1,3,2-dioxaborolane (2.0 g, 52% yield) as yellow oil. ¹H NMR (400 MHz, Methanol-d₄) δ 7.72 (d, J=8.0 Hz, 1H), 7.36 (t, J=73.2 Hz, 1H), 7.12 (t, J=74.4 Hz, 1H), 7.12-7.08 (m, 1H), 7.02-6.97 (m, 1H), 1.29 (s, 12H).

### Intermediate 2g. 2-(4-bromo-2-(difluoromethoxy)phenyl)-4,4,5,5-tetramethyl-1,3,2-dioxaborolane

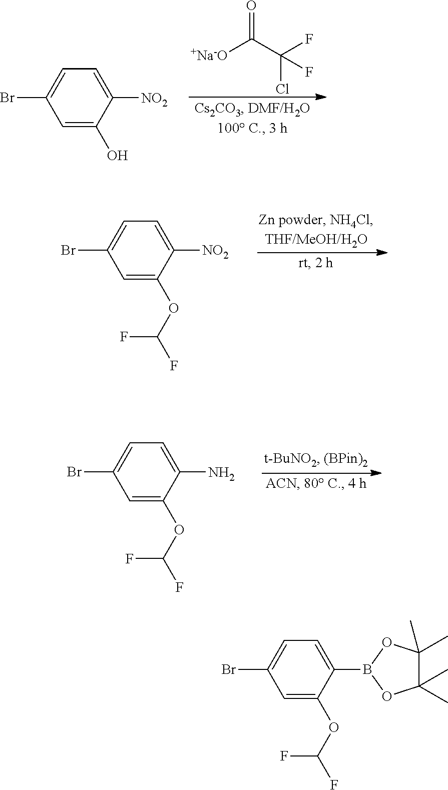

### Step 1. 4-Bromo-2-(difluoromethoxy)-1-nitrobenzene

To a solution of 5-bromo-2-nitrophenol (5.7 g, 26.15 mmol) in DMF (135 mL)/H₂O (15 mL) was added Cs₂CO₃ (17.0 g, 52.29 mmol) and sodium 2-chloro-2,2-difluoroacetate (10.0 g, 65.37 mmol). The mixture was stirred for 15 minutes at room temperature and then heated to 100° C. for 16 hours. The reaction mixture was cooled to room temperature and partitioned between water (500 mL) and EA (200 mL). The aqueous phase was extracted with EA (2×200 mL). The combined organic extracts were washed with brine (300 mL), dried over Na₂SO₄, filtered, and concentrated. The residue was purified by silica gel column (0-20% EA in PE) to obtain 4-bromo-2-(difluoromethoxy)-1-nitrobenzene as a yellow oil (4.0 g, 57% yield). 1H NMR (400 MHz, DMSO-d₆) δ 8.05 (d, J=8.8 Hz, 1H), 7.83 (d, J=1.6 Hz, 1H), 7.74 (dd, J=8.8, J=1.6 Hz, 1H), 7.43 (t, J=72.4 Hz, 1H).

### Step 2. 4-Bromo-2-(difluoromethoxy) aniline

To a solution of 4-bromo-2-(difluoromethoxy)-1-nitrobenzene (1.0 g, 3.73 mmol) in THE (10 mL)/H₂O (5 mL) was added the solution of NH₄Cl (1.6 g, 29.85 mmol) in H₂O (10 mL), Zn (1.95 g, 29.85 mmol). The mixture was stirred at rt for 2 h, then filtered through a pad of celite and rinsed with EA. The filtrate was extracted with EA (30 mL×2). The organic extracts were dried over Na₂SO₄, decanted, concentrated, and purified by silica gel column (0-10% EA in PE) to afford 4-bromo-2-(difluoromethoxy) aniline as a yellow oil (750 mg, 84% yield). MS m/z 236.0 [M−1]⁻; 1H NMR (400 MHz, DMSO-d₆) δ 7.15 (s, 1H), 7.11 (dd, J=8.8, 2.0 Hz, 1H), 7.09 (t, J=74.0 Hz, 1H), 6.73 (d, J=8.8 Hz, 1H), 5.28 (s, 2H).

### Step 3. 2-(4-Bromo-2-(difluoromethoxy) phenyl)-4,4,5,5-tetramethyl-1,3,2-dioxaborolane

To a solution of 4-bromo-2-(difluoromethoxy) aniline (13.0 g, 54.61 mmol) in MeCN (300 mL) was added t-BuNO₂ (8.45 g, 81.92 mmol), (BPin)₂ (15.26 g, 60.08 mmol) at 0° C. The mixture was stirred at 80° C. for 4 h, then concentrated and purified by silica gel column (0-5% EA in PE) to afford 2-(4-bromo-2-(difluoromethoxy) phenyl)-4,4,5,5-tetramethyl-1,3,2-dioxaborolane as a yellow oil (5.4 g, 28% yield). 1H NMR (400 MHz, DMSO-d₆) δ 7.60 (d, J=8.0 Hz, 1H), 7.51 (dd, J=8.0, J=1.6 Hz, 1H), 7.42 (s, 1H), 7.15 (t, J=74.2 Hz, 1H), 1.29 (s, 12H).

The intermediates below were prepared according to the procedure of Intermediate 2e-2g by substituting the appropriate starting materials, reagents and reaction conditions.

| Structure | Spectral Data |
|---|---|
| 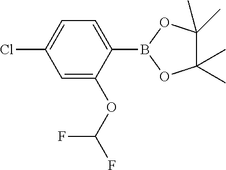 [SMILES: CC1(C)OB(c2ccc(Cl)cc2OC(F)F)OC1(C)C] | MS m/z 304.5 [M + H]^{+; 1}H NMR (400 MHz, DMSO-d₆) δ 7.68 (d, J = 8.4 Hz, 1H), 7.38 (d, J = 7.6 Hz, 1H), 7.30 (s, 1H), 7.16 (t, J = 74.4Hz, 1H), 1.30 (s, 12H). |
| 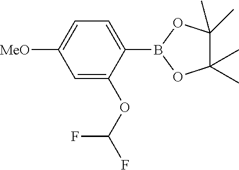 [SMILES: COc1ccc(B2OC(C)(C)C(C)(C)O2)c(OC(F)F)c1] | ¹H NMR (400 MHz, DMSO-d₆) δ 7.60 (d, J = 8.4 Hz, 1H), 7.05 (t, J = 70.4 Hz, 1H), 6.86 (dd, J = 8.6, 2.0 Hz, 1H), 6.70 (d, J = 2.0 Hz, 1H), 3.80 (s, 3H), 1.27 (s, 12H). |
| 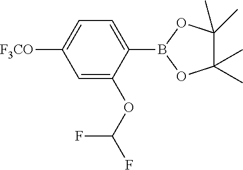 [SMILES: CC1(C)OB(c2ccc(OC(F)(F)F)cc2OC(F)F)OC1(C)C] | ¹H NMR (400 MHz, CDCl₃) δ 7.79 (d, J = 8.3 Hz, 1H), 7.11 (d, J = 8.2 Hz, 1H), 7.02 (s, 1H), 6.53 (t, J = 74.7 Hz, 1H), 1.26 (s, 12H). |

### Intermediate 2h: 2-(2-cyclopropyl-6-fluoro-4-methoxyphenyl)-4,4,5,5-tetramethyl-1,3,2-dioxaborolane

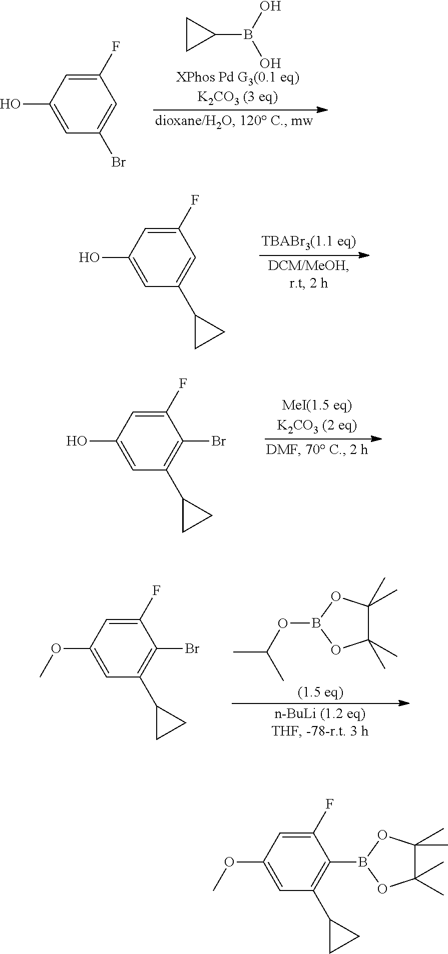

### Step 1. 3-cyclopropyl-5-fluorophenol

To a solution of 3-bromo-5-fluorophenol (5.0 g, 26.3 mmol, 1.0 eq.) in dioxane (40 mL) and water (10 mL) was added cyclopropylboronic acid (4.5 g, 52.6 mmol, 2.0 eq.), Pd(dppf)Cl₂ (377 mg, 0.52 mmol, 0.2 eq.) and cesium carbonate (25.7 g, 78.9 mmol, 3.0 eq.). The resulting mixture was stirred at 100° C. for 16 hours under nitrogen atmosphere, then diluted with ethyl acetate (200 mL), washed with water (200 mL). The aqueous phase was adjusted to pH-4 by addition of 6M HCl, then extracted with ethyl acetate (3×100 mL). The organic phase was dried with sodium sulfate, filtered, and concentrated under reduced pressure. The residue was purified by flash column chromatography (10% ethyl acetate in petroleum ether) to afford 4-bromo-3-cyclopropyl-5-fluorophenol (3.1 g, 77.5% yield) as a yellow oil. 1H NMR (400 MHz, Methanol-d₄) δ 6.31 (t, J=1.8 Hz, 1H), 6.28-6.25 (m, 1H), 6.25-6.22 (m, 1H), 1.85-1.76 (m, 1H), 0.97-0.88 (m, 2H), 0.66-0.58 (m, 2H).

### Step 2. 4-bromo-3-cyclopropyl-5-fluorophenol

To a solution of 4-bromo-3-cyclopropyl-5-fluorophenol (3 g, 19.7 mmol, 1.0 eq.) in dichloromethane (15 mL) and methanol (15 mL) was added tetrabutylammonium tribromide (10.4 g, 21.7 mmol, 1.1 eq.). The resulting mixture was stirred at room temperature for 2 h. Water (100 mL) was then added. The mixture was extracted with dichloromethane (3×50 mL). The organic phase was washed with brine (50 mL), dried with sodium sulfate, filtered and concentrated under reduced pressure. The residue was purified by flash column chromatography (10% ethyl acetate in petroleum ether) to afford 4-bromo-3-cyclopropyl-5-fluorophenol (3.9 g, 85.7% yield) as a yellow oil. 1H NMR (400 MHz, Methanol-d₄) δ 6.50-6.42 (m, 1H), 6.27-6.20 (m, 1H), 2.23-2.09 (m, 1H), 1.03-0.97 (m, 2H), 0.66-0.59 (m, 2H).

### Step 3. 2-bromo-1-cyclopropyl-3-fluoro-5-methoxybenzene

To a solution of 4-bromo-3-cyclopropyl-5-fluorophenol (3.9 g, 16.9 mmol, 1.0 eq.) in N,N-dimethylformamide (40 mL) was added methyl iodide (1.58 mL, 25.4 mmol, 1.5 eq.) and potassium carbonate (4.67 g, 33.8 mmol, 2.0 eq.). The resulting mixture was stirred at 70° C. for 2 h. The reaction mixture was diluted with ethyl acetate (150 mL) and washed with water (3×100 mL) and brine (100 mL). The organic phase was dried with sodium sulfate, filtered and concentrated under reduced pressure. The residue was purified by flash column chromatography (3% ethyl acetate in petroleum ether) to afford 2-bromo-1-cyclopropyl-3-fluoro-5-methoxybenzene (3.1 g, 74.8% yield) as a pink oil. 1H NMR (400 MHz, Methanol-d₄) δ6.69-6.55 (m, 1H), 6.48-6.28 (m, 1H), 3.87-3.72 (m, 3H), 2.26-2.08 (m, 1H), 1.03-0.93 (m, 2H), 0.77-0.59 (m, 2H).

### Step 4. 2-(2-cyclopropyl-6-fluoro-4-methoxyphenyl)-4,4,5,5-tetramethyl-1,3,2-dioxaborolane

To a solution of 2-bromo-1-cyclopropyl-3-fluoro-5-methoxybenzene (3.0 g, 12.2 mmol, 1.0 eq.) in tetrahydrofuran (30 mL) was added n-butyllithium (6.3 mL, 15.9 mmol, 1.3 eq., 2.5M in hexane) at −78° C. The resulting mixture was stirred at −78° C. for 30 min, then 2-isopropoxy-4,4,5,5-tetramethyl-1,3,2-dioxaborolane was added dropwise (2.97 mL, 14.6 mmol, 1.2 eq.). The resulting mixture was allowed to warm to room temperature and stirred for another 2 h. The reaction was quenched by water (100 mL), and the mixture was extracted with ethyl acetate (4×50 mL). The organic phase was dried with sodium sulfate, filtered and concentrated under reduced pressure. The residue was purified by flash column chromatography (3% ethyl acetate in petroleum ether) to afford 2-(2-cyclopropyl-6-fluoro-4-methoxyphenyl)-4,4,5,5-tetramethyl-1,3,2-dioxaborolane (1.9 g, 53.3% yield) as a light yellow oil. MS m/z 293.2 [M+H]⁺; 1H NMR (400 MHz, Methanol-d₄) δ 6.52-6.37 (m, 1H), 6.31-6.17 (m, 1H), 3.79-3.69 (m, 3H), 2.34-2.19 (m, 0.8H), 2.00-1.90 (m, 0.16H), 1.39-1.33 (m, 12H), 0.95-0.84 (m, 2H), 0.69-0.60 (m, 2H).

### Intermediate 2i. 2-(4-Methoxy-2-(2,2,2-trifluoroethoxy)phenyl)-4,4,5,5-tetramethyl-1,3,2-dioxaborolane

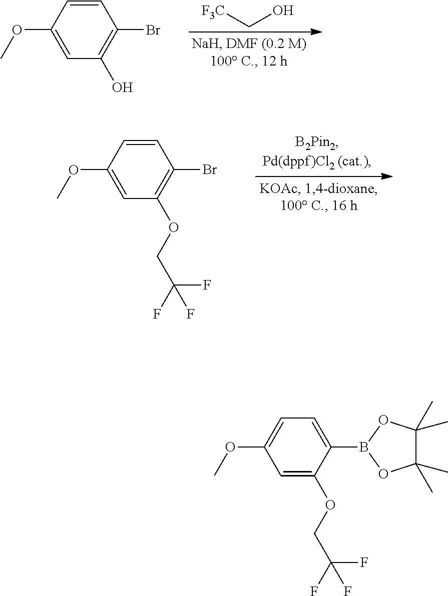

### Step 1. 1-bromo-4-methoxy-2-(2,2,2-trifluoroethoxy)benzene

To a solution of NaH (2.2 g, 53.6 mmol, 60% in oil, 2.2 eq.) in DMF (20 mL) and 2,2,2-trifluoroethan-1-ol (4.9 g, 48.8 mmol, 2.0 eq.) was added dropwise at 0° C. under N₂. The mixture was stirred at 0° C. for 0.5 hours under N₂ and 1-bromo-2-fluoro-4-methoxybenzene (5.0 g, 24.4 mmol, 1.0 eq.) was added. The mixture was allowed to warm to 100° C. and stirred for 16 hours under N₂, then poured into water (100 mL) and extracted with dichloromethane (3×50 mL). The combined organic extracts were washed with brine (50 mL), dried with sodium sulfate, filtered and concentrated under reduced pressure. The residue was purified by flash column chromatography (0-7% ethyl acetate in petroleum ether) to afford 1-bromo-4-methoxy-2-(2,2,2-trifluoroethoxy)benzene (3.5 g, 12.3 mmol, 50.4% yield) a colorless oil. 1H NMR (400 MHz, DMSO-d₆) δ 7.49 (d, J=8.8 Hz, 1H), 6.85 (d, J=2.8 Hz, 1H), 6.61 (dd, J=8.8, 2.8 Hz, 1H), 4.86 (q, J=8.8 Hz, 2H), 3.78 (s, 3H).

### Step 2. 2-(4-Methoxy-2-(2,2,2-trifluoroethoxy)phenyl)-4,4,5,5-tetramethyl-1,3,2-dioxaborolane

To a solution of 1-bromo-4-methoxy-2-(2,2,2-trifluoroethoxy)benzene (3.50 g, 12.3 mmol, 1.0 eq.) in dioxane (40 mL) was added 4,4,4′,4′,5,5,5′,5′-octamethyl-2,2′-bi(1,3,2-dioxaborolane) (3.7 g, 14.7 mmol, 1.2 eq.), Pd(dppf)Cl₂ (898.4 mg, 1.2 mmol, 0.1 eq.) and KOAc (3.6 g, 36.8 mmol, 3.0 eq.). The resulting mixture was stirred at 100° C. for 16 hours under nitrogen atmosphere Then cooled, diluted with ethyl acetate (200 mL), washed with water (200 mL), and extracted with ethyl acetate (3×100 mL). The organic phase was dried with sodium sulfate, filtered and concentrated under reduced pressure. The residue was purified by flash column chromatography (0-10% ethyl acetate in petroleum ether) to afford 2-(4-methoxy-2-(2,2,2-trifluoroethoxy)phenyl)-4,4,5,5-tetramethyl-1,3,2-dioxaborolane (1.5 g, 4.5 mmol, 36.8% yield) as a white solid. MS m/z 333.1[M+H]+; 1H NMR (400 MHz, Methanol-d₄) δ 7.56 (d, J=8.4 Hz, 1H), 6.63 (dd, J=8.0, 2.0 Hz, 1H), 6.54 (d, J=2.0 Hz, 1H), 4.47 (q, J=8.4 Hz, 2H), 3.81 (s, 3H), 1.32 (s, 12H).

### Intermediate 2j. 2-(4-methoxy-2-(trifluoromethoxy)phenyl)-4,4,5,5-tetramethyl-1,3,2-dioxaborolane

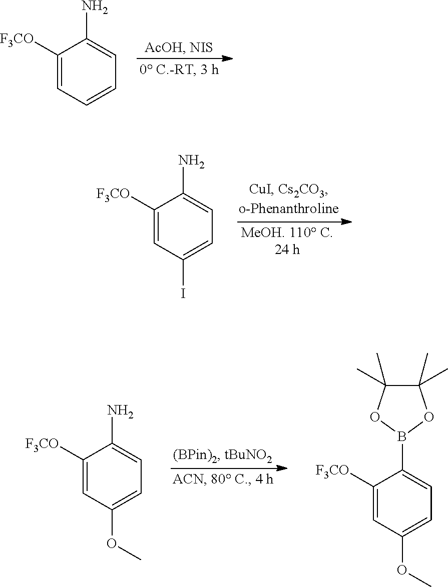 [SMILES: Nc1ccccc1OC(F)(F)F ; Nc1ccc(I)cc1OC(F)(F)F ; COc1ccc(N)c(OC(F)(F)F)c1 ; COc1ccc(B2OC(C)(C)C(C)(C)O2)c(OC(F)(F)F)c1]

### Step 1. 4-Iodo-2-(trifluoromethoxy)aniline

To a solution of 2-(trifluoromethoxy)aniline (29 g, 163.73 mmol, 1.0 eq.) in acetic acid (80 mL) was added NIS (40.52 g, 180.10 mmol, 1.1 eq.) slowly at 0° C. The mixture was stirred for 3 h at room temperature. The reaction solution was concentrated in vacuo to remove acetic acid and then the reaction mixture was diluted with water (200 mL) and extracted with ethyl acetate (200 mL×3). The combined organic layers were washed with brine, dried over anhydrous Na₂SO₄ and concentrated. The residue was purified by flash chromatography on silica gel (EA in PE=0%-20) to afford 4-iodo-2-(trifluoromethoxy)aniline (40.6 g, 81.8% yield) as a red solid. MS m/z 303.9 [M+1]⁺; ¹H NMR (400 MHz, DMSO-d₆) δ 7.35-7.32 (m, 2H), 6.66 (d, J=8.4 Hz, 1H), 5.59 (s, 2H).

### Step 2. 4-Methoxy-2-(trifluoromethoxy)aniline

To a solution of 4-iodo-2-(trifluoromethoxy)aniline (23.8 g, 78.54 mmol, 1.0 eq.) in MeOH (200 mL) was added 1,10-phenanthroline (1.42 g, 7.85 mmol, 0.1 eq.),Cs₂CO₃ (51.18 g, 157.08 mmol, 2.0 eq.) and CuI (0.748 g, 3.93 mmol, 0.05 eq.) under N₂ at rt. The reaction mixture was stirred for 16 h at 110° C. in an autoclave reactor. The reaction solution was concentrated in vacuo to remove MeOH and then the reaction mixture was diluted with water (200 mL) and extracted with ethyl acetate (250 mL×3). The combined organic layers were washed with brine, dried over anhydrous Na₂SO₄ and concentrated. The residue was purified by flash chromatography on silica gel (EA in PE=0%˜10%) to afford 4-methoxy-2-(trifluoromethoxy)aniline (11 g, 67.61% yield) as yellow oil. MS m/z 208.2 [M+1]⁺; ¹H NMR (400 MHz, Methanol-d₄) δ 6.91-6.67 (m, 3H), 3.71 (s, 3H).

### Step 3. 2-(4-Methoxy-2-(trifluoromethoxy)phenyl)-4,4,5,5-tetramethyl-1,3,2-dioxaborolane

To a solution of 4-methoxy-2-(trifluoromethoxy)aniline (11 g, 53.10 mmol, 1.0 eq.) in ACN (25 mL) was added tBuNO₂ (8.21 g, 79.65 mmol, 1.5 eq.) and (BPin)₂ (16.18 g, 63.72 mmol, 1.2 eq.) at 0° C. The reaction mixture was stirred for 4 h at 80° C. under N₂ atmosphere, then cooled, and concentrated in vacuo before diluted with water (200 mL) and extracted with ethyl acetate (150 mL×3). The combined organic layers were washed with brine, dried over anhydrous Na₂SO₄ and concentrated. The residue was purified by flash chromatography on silica gel (EA in PE=0%˜2%) to afford 2-(4-methoxy-2-(trifluoromethoxy)phenyl)-4,4,5,5-tetramethyl-1,3,2-dioxaborolane (5.6 g, 33.15% yield) as yellow oil. MS m/z 319.0 [M+1]⁺; ¹H NMR (400 MHz, DMSO-d₆) δ 7.71 (d, J=8.4 Hz, 1H), 7.08-7.03 (m, 1H), 6.92 (s, 1H), 3.87 (s, 3H), 1.32 (s, 12H).

### Intermediate 3a: (3R)-1-[2-[tert-Butyl(dimethyl)silyl]oxyethyl]piperidin-3-amine

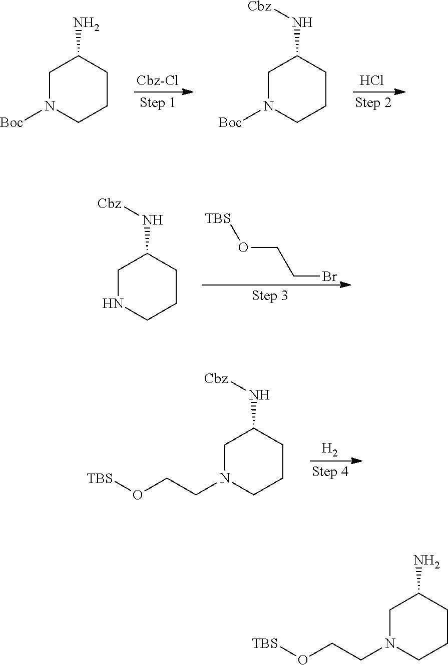

### Step 1. tert-Butyl (3R)-3-(benzyloxycarbonylamino)piperidine-1-carboxylate

To a solution of tert-butyl (R)-3-aminopiperidine-1-carboxylate (30 g, 0.15 mol) in THE (330 mL) and water (83 mL) was added Na₂CO₃ (43 g, 0.31 mol). Then Cbz-Cl (34, 0.199 mol) was added dropwise to the mixture with ice bath, monitored by TLC. After 5 hours, the reaction mixture was extracted with EA, dried over Na₂SO₄ and evaporated in vacuo. The crude product was purified via flash chromatography (PE:EA=4:1) to give tert-butyl (3R)-3-(benzyloxycarbonylamino) piperidine-1-carboxylate (35 g, 84% Yield). MS m/z 235.1 [M−Boc+H]⁺

### Step 2. Benzyl N-[(3R)-3-piperidyl]carbamate

To a solution of tert-butyl (3R)-3-(benzyloxycarbonylamino)piperidine-1-carboxylate (35 g, 0.104 mol) in 450 mL DCM was added HCl (350 mL, 4M in 1,4-dioxane), monitored by TLC. After 2 h, the solvent was removed in vacuo to give crude benzyl N-[(3R)-3-piperidyl]carbamate (25 g, 100% yield) which was used for next step without purification. MS m/z 234.1 [M+H]⁺.

### Step 3. Benzyl N-[(3R)-1-[2-[tert-butyl(dimethyl)silyl]oxyethyl]-3-piperidyl]carbamate

To a solution of benzyl N-[(3R)-3-piperidyl]carbamate (25 g, 0.106 mol) in CH₃CN (680 mL) was added Cs₂CO₃ (175 g, 0.537 mol) and (2-bromoethoxy)(tert-butyl)dimethylsilane (40 g, 0.167 mol). The reaction mixture was stirred at 90° C. for 16 h, then filtered and the solvent was removed in vacuo. The crude product was purified via flash chromatography to give the product benzyl N-[(3R)-1-[2-[tert-butyl(dimethyl)silyl]oxyethyl]-3-piperidyl]carbamate (22.5 g, 53% yield). MS m/z 393.2 [M+H]⁺.

### Step 4. (3R)-1-[2-[tert-Butyl(dimethyl)silyl]oxyethyl]piperidin-3-amine

To a solution of benzyl N-[(3R)-1-[2-[tert-butyl(dimethyl)silyl]oxyethyl]-3-piperidyl]carbamate (22.5 g, 0.057 mol) in MeOH (60 mL) was added Pd/C (4.5 g, 20%). The system was evacuated and refilled with hydrogen. Then the mixture stirred at R.T overnight. After reaction, the mixture was filtered, the solvent was removed in vacuo and the crude product was purified via flash chromatography to give the product (3R)-1-[2-[tert-butyl(dimethyl)silyl]oxyethyl]piperidin-3-amine (12 g, 81% yield). MS m/z 259.2 [M+H]⁺. ¹H NMR (400 MHz, CDCl₃) δ 3.68 (t, J=6.6 Hz, 2H), 2.85-2.74 (m, 2H), 2.67-2.58 (m, 1H), 2.46 (t, J=6.4 Hz, 2H), 2.11-2.01 (m, 1H), 1.94-1.83 (m, 1H), 1.83-1.71 (m, 4H), 1.68-1.58 (m, 1H), 1.56-1.43 (m, 1H), 1.09-0.96 (m, 1H), 0.88-0.79 (m, 9H), 0.04-(−) 0.04 (m, 6H).

### Intermediate 3b: (R)-1-Ethylpiperidin-3-amine hydrochloride

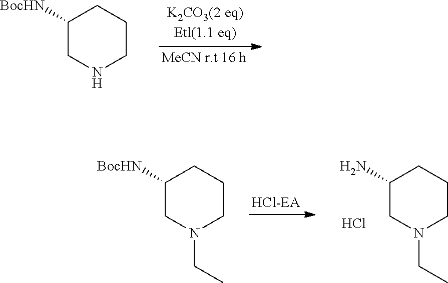

### Step 1. tert-Butyl (R)-(1-ethylpiperidin-3-yl)carbamate

To a solution of tert-butyl (R)-piperidin-3-ylcarbamate (4 g, 20 mmol) in MeCN (40 mL) was added K₂CO₃ (2.76 g, 20 mmol) and Mel (3.12 g, 20 mmol) at 20° C. under N₂. The mixture was stirred at 20° C. for 16 hours. TLC showed the reaction was completed. The reaction mixture was poured into water (100 mL) and extracted with EA (100 mL×2). The combined organic layers were washed with brine (100 mL×2). dried over Na₂SO₄, filtered and concentrated. Purified by column (DCM:MeOH=0-10%) to give tert-butyl (R)-(1-ethylpiperidin-3-yl)carbamate (3.6 g, 15.8 mmol, 78.9% yield) as yellow oil. ¹H NMR (400 MHz, DMSO-d₆) δ 6.65 (d, J=7.8 Hz, 1H), 3.32 (s, 1H), 2.70 (dd, J=41.0, 9.7 Hz, 2H), 2.29 (q, J=7.1 Hz, 2H), 1.83-1.53 (m, 4H), 1.48-1.40 (m, 1H), 1.37 (s, 9H), 1.11 (qd, J=11.8, 3.7 Hz, 1H), 0.96 (t, J=7.2 Hz, 3H).

### Step 2. (R)-1-Ethylpiperidin-3-amine hydrochloride

To a solution of tert-butyl (R)-(1-ethylpiperidin-3-yl)carbamate (3.6 g, 15.8 mmol) in MeOH (10 mL) was added HCl (30 mL, 3 M in EA) at RT. The mixture was stirred at 20° C. for 16 hours. TLC showed the reaction was completed. The reaction mixture was concentrated to give(R)-1-ethylpiperidin-3-amine hydrochloride (3.03 g, 15.0 mmol, 95.0% yield) as yellow oil. MS m/z 129.2 [M+H]⁺; ¹H NMR (400 MHz, Methanol-d₄) δ 3.83-3.59 (m, 3H), 3.36-3.30 (m, 2H), 3.18-2.96 (m, 2H), 2.25 (d, J=12.7 Hz, 1H), 2.15 (d, J=14.9 Hz, 1H), 2.07-1.92 (m, 1H), 1.76-1.72 (m, 1H), 1.43 (t, J=7.3 Hz, 3H).

### Intermediate 9c: (R)-1-(3-Aminopiperidin-1-yl)-2-methylpropan-2-ol

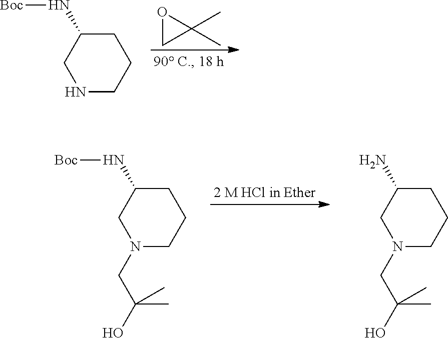

### Step-1: tert-Butyl (R)-(1-(2-hydroxy-2-methylpropyl)piperidin-3-yl)carbamate

A mixture of tert-butyl (R)-piperidin-3-ylcarbamate (700 mg, 3.5 mmol, 1.0 eq.), and 2,2-dimethyloxirane (800 mg, 11.1 mmol, 3.2 eq.) was stirred at 90° C. for 18 h. The crude reaction mixture was allowed to cool to room temperature and diluted with DCM (20 mL). The organic phase was washed with saturated aqueous NaHCO₃ solution (4 mL×2) followed by water and brine. The organic phase was dried over MgSO₄, filtered, and concentrated in vacuo. The crude material was used then for the following step. MS m/z 273.3 [M+H]⁺.

### Step-2: (R)-1-(3-Aminopiperidin-1-yl)-2-methylpropan-2-ol

The crude compound from previous step was dissolved in DCM (5 mL) and then 2M HCl solution in ether (10 mL) was added slowly at rt while the mixture was vigorously stirred. The reaction mixture was stirred at rt for overnight and concentrated in vacuo to afford a light brown solid (520 mg) of HCl salt. The amine was used without further purification. MS m/z 173.2 [M+H]⁺.

The intermediates below were prepared according to the procedure of Intermediate 3b-c or c by substituting the appropriate starting materials, reagents and reaction conditions.

| Structure | Spectral Data |
|---|---|
| 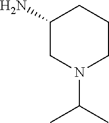 | MS m/z 243.2 [M + H]⁺; ¹H NMR (400 MHz, Methanol-d₄) δ 3.83-3.72 (m, 1H), 3.71-3.61 (m, 2H), 3.56-3.48 (m, 1H), 3.15-3.03 (m, 2H), 2.30-2.19 (m, 1H), 2.18-2.10 (m, 1H), 2.09-1.97 (m, 1H), 1.81-1.66 (m, 1H), 1.43 (d, J = 6.7 Hz, 6H). |
| 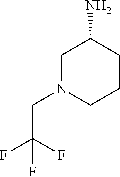 [SMILES: N[C@@H]1CCCN(CC(F)(F)F)C1] | MS m/z 183.2 [M + H]⁺; ¹H NMR (400 MHz, Methanol-d₄) δ 4.00 (q, J = 9.3 Hz, 2H), 3.65 (ddd, J = 22.7, 13.2, 8.3 Hz, 2H), 3.39 (d, J = 12.2 Hz, 1H), 3.26-3.06 (m, 2H), 2.09 (dtd, J = 23.9, 8.6, 4.2 Hz, 2H), 2.01-1.85 (m, 1H), 1.72 (dtd, J = 13.8, 10.1, 3.7 Hz, 1H). |
| 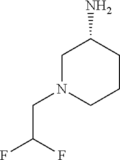 [SMILES: N[C@@H]1CCCN(CC(F)F)C1] | ¹H NMR(400 MHz, Methanol-d₄) δ 6.48 (tt, J = 53.5, 3.7 Hz, 1H), 3.84-3.64 (m, 4H), 3.59 (d, J = 11.9 Hz, 1H), 3.22 (dt, J = 12.2, 7.1 Hz, 2H), 2.15 (dtd, J = 15.1, 7.9, 3.8 Hz, 2H), 2.07-1.93 (m, 1H), 1.71 (ddd, J = 15.2, 11.9, 3.8 Hz, 1H). |
| 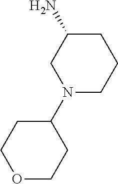 [SMILES: N[C@@H]1CCCN(C2CCOCC2)C1] | MS m/z 185.2[M + H]⁺ |
| 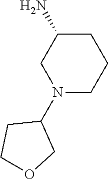 [SMILES: N[C@@H]1CCCN(C2CCOC2)C1] | MS m/z 171.2[M + H]⁺ |
| 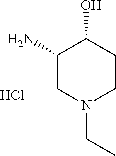 | MS m/z 145.2[M + H]⁺ |

### Intermediate 3d: (R)-1-(2-(Difluoromethoxy)ethyl)piperidin-3-amine hydrochloride

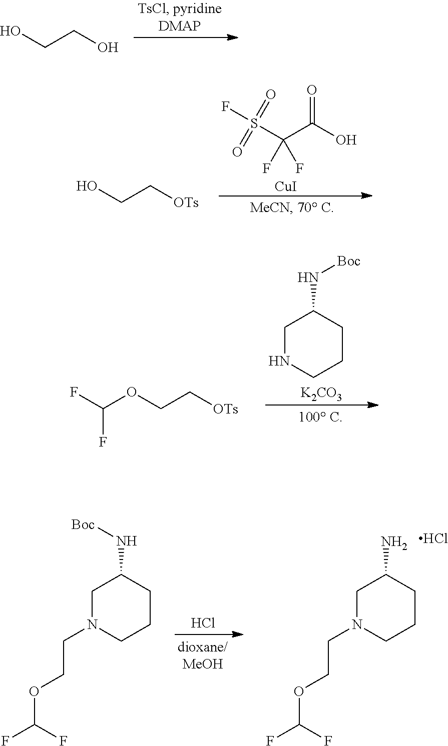

### Step 1: 2-Hydroxyethyl 4-methylbenzenesulfonate

A mixture of ethylene glycol (2.5 g, 40 mmol), TsCl (1.90 g, 10 mmol), pyridine (1.1 g, 12 mmol) and DMAP (12 mg, 0.1 mmol) was stirred at rt for 18 h. The reaction mixture was partitioned between DCM (20 mL) and 0.5 M hydrochloric acid. The organic layer was dried (Na₂SO₄), filtered, and concentrated. Purification by chromatography on SiO₂ (EtOAc:hexanes, 0 to 60%) gave a colorless oil (1.32 g, 78%). ¹H NMR (400 MHz, CDCl₃) δ 7.82 (d, J=8.25 Hz, 2H), 7.37 (d, J=8.13 Hz, 2H), 4.15 (t, J=4.50 Hz, 2H), 3.83 (t, J=4.63 Hz, 2H), 2.46 (s, 3H), 2.00 (br s, 1H).

### Step 2. 2-(Difluoromethoxy)ethyl 4-methylbenzenesulfonate

To a stirred solution of 2-hydroxyethyl 4-methylbenzenesulfonate (1.70 g, 7.86 mmol) in MeCN (13 mL) was added copper (I) iodide (0.300 g, 1.57 mmol). The resulting mixture was stirred at 70° C. and treated with 2,2-difluoro-2-fluorosulfonyl-acetic acid (2.80 g, 15.7 mmol) as a solution in MeCN (10 mL) dropwise over a period of 25 min. The resulting mixture was treated with anhydrous Na₂SO₄ (small scoop) and stirring continued for 1.5 h. The mixture was then cooled to rt, diluted with Et₂O and washed with brine, a 1:1 mixture of brine: water (2×), and brine. The organic phase was dried (Na₂SO₄), filtered and concentrated. Purification by chromatography on SiO₂ (EtOAc:hexanes, 0-25%) gave a pale yellow oil (0.759 g, 36%). ¹H NMR (400 MHz, CDCl₃) δ 7.79 (d, J=8.25 Hz, 2H), 7.49 (d, J=8.13 Hz, 2H), 6.65 (t, J=75.04 Hz, 1H), 4.22-4.16 (m, 2H), 4.04-3.98 (m, 2H), 2.43 (s, 3H)

### Step 3 and 4: (R)-1-(2-(Difluoromethoxy)ethyl)piperidin-3-amine hydrochloride

t-Butyl N-[(3R)-3-piperidyl]carbamate (0.500 g, 2.50 mmol) in DMF (10 mL) was added 2-(difluoromethoxy)ethyl 4-methylbenzenesulfonate (0.764 g, 2.87 mmol) and K₂CO₃ (0.690 g, 4.99 mmol). The mixture was stirred at 100° C. for 2 h, then diluted with DCM/iPrOH (9:1) and washed with water, brine, dried over Na₂SO₄, filtered and concentrated. Purification by chromatography on SiO₂ (EtOAc:hexanes, 5 to 60%) gave a pale yellow oil (0.709 g). The oil was dissolved in MeOH (2.0 mL), treated with HCl/dioxane (4.0 M, 3 mL) and stirred for 4 h. The mixture was concentrated, resuspended in ether and filtered to give a white solid (0.371 g, 67%). ¹H NMR (400 MHz, D₂O) δ 6.45 (t, J=73.6 Hz, 1H), 4.26 (t, J=5.2 Hz, 2H), 3.81 (d, J=11.2 Hz, 1H), 3.67-3.58 (m, 2H), 3.55 (t, J=4.8 Hz, 2H), 3.15-3.02 (m, 2H), 2.21 (d, J=12.4 Hz, 1H), 2.11 (d, J=15.2 Hz, 1H), 1.90-1.79 (m, 1H), 1.70-1.60 (m, 1H).

### Intermediate 3f: (3R,5R)-5-Fluoro-1-methylpiperidin-3-amine hydrochloride

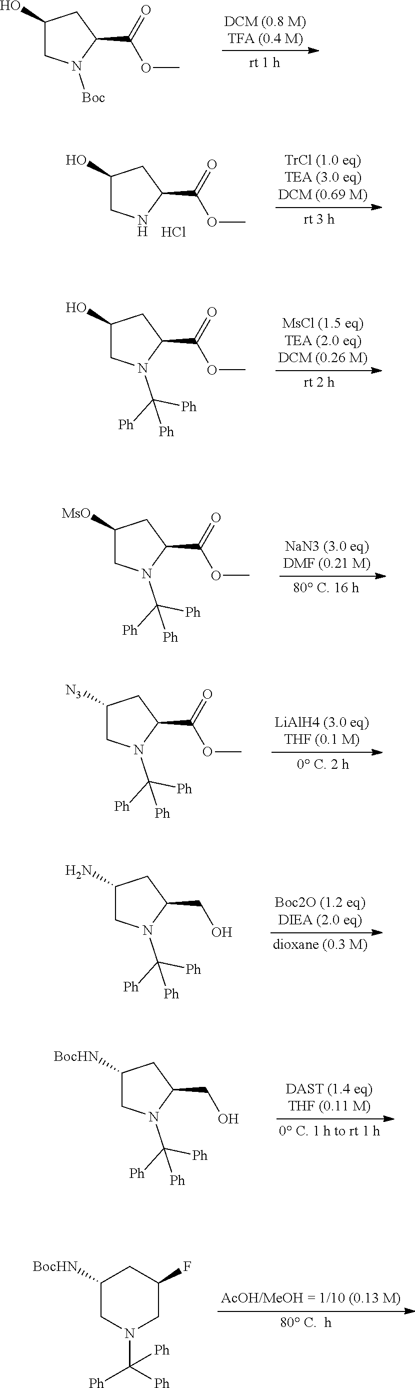

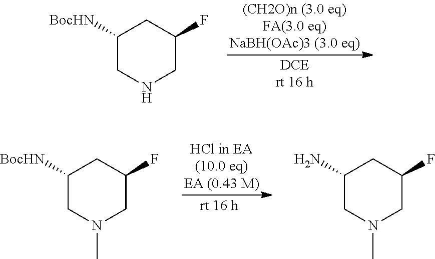

### Step 1. Methyl (2S,4S)-4-hydroxypyrrolidine-2-carboxylate

1-(tert-Butyl) 2-methyl (2S,4S)-4-hydroxypyrrolidine-1,2-dicarboxylate (10.0 g, 40.8 mmol) was dissolved in DCM (50 mL) and cooled to 0° C. TFA (10 mL, 132.3 mmol) was slowly added and the mixture was warmed to room temperature and stirred for 1 h. The mixture was concentrated in vacuo to give crude methyl (2S,4S)-4-hydroxypyrrolidine-2-carboxylate as yellow oil, which was used to next step without further purification. MS m/z 146.1 [M+H]⁺.

### Step 2. Methyl (2S,4S)-4-hydroxy-1-tritylpyrrolidine-2-carboxylate

To a solution of crude methyl (2S,4S)-4-hydroxypyrrolidine-2-carboxylate from above step in DCM (50 mL) was added TEA (12.4 g, 123 mmol) and Ph₃CCl (11.4 g, 40.9 mmol,). The reaction mixture was stirred for 2 h at rt. The mixture was poured into water and extracted with DCM (30 mL×2). The organic layer was washed with brine, dried over anhydrous Na₂SO₄, and evaporated in vacuo. The residue was purified by flash chromatography on silica gel (PE:EA=10:1 to 5:1) to obtain methyl (2S,4S)-4-hydroxy-1-trityl-pyrrolidine-2-carboxylate (7.60 g, 48% yield for two steps) as a white solid. ¹H NMR (400 MHz, CDCl₃) δ 7.60-7.50 (m, 6H), 7.32-7.23 (m, 6H), 7.21-7.13 (m, 3H), 3.92-3.80 (m, 3H), 3.65 (s, 3H), 3.46 (d, J=11.5 Hz, 1H), 2.80 (dd, J=11.5, 3.8 Hz, 1H), 1.62 (d, J=13.8 Hz, 1H), 1.38-1.11 (m, 1H).

### Step 3. Methyl (2S,4S)-4-((methylsulfonyl)oxy)-1-tritylpyrrolidine-2-carboxylate

To a solution of methyl (2S,4S)-4-hydroxy-1-trityl-pyrrolidine-2-carboxylate (10.0 g, 25.8 mmol) in DCM (100 mL) was added TEA (7.90 g, 78.0 mmol) and MsCl (4.50 g, 39.0 mmol). The reaction mixture was stirred for 2 h at rt. The mixture was diluted with water and extracted with DCM (80 mL×2). The organic layer was washed with brine, dried over anhydrous Na₂SO₄, and evaporated in vacuo to give crude methyl (2S,4S)-4-methylsulfonyloxy-1-trityl-pyrrolidine-2-carboxylateas yellow oil, which was used in the next step directly.

### Step 4. Methyl (2S,4R)-4-azido-1-tritylpyrrolidine-2-carboxylate

To a solution of methyl (2S,4S)-4-methylsulfonyloxy-1-trityl-pyrrolidine-2-carboxylate from above step in DMF (140 mL) was added sodium azide (7.80 g, 120 mmol). The reaction mixture was stirred for 16 h at 80° C., then diluted with water and extracted with EA (3×100 mL). The combined organic layers were washed with water, brine, dried over anhydrous Na₂SO₄ and concentrated. The residue was purified by flash chromatography on silica gel (PE:EA=0% 10%) to give methyl (2S,4R)-4-azido-1-trityl-pyrrolidine-2-carboxylate (8.00 g, 64.5% yield for two steps) as yellow oil. ¹H NMR (400 MHz, CDCl₃) δ 7.58-7.51 (m, 6H), 7.33-7.24 (m, 6H), 7.18 (t, J=7.3 Hz, 3H), 4.20-4.06 (m, 1H), 3.91 (d, J=8.9 Hz, 1H), 3.72 (dd, J=10.3, 7.6 Hz, 1H), 3.63 (s, 3H), 2.63 (dd, J=10.3, 7.8 Hz, 1H), 1.90-1.79 (m, 1H), 0.94-0.88 (m, 1H).

### Step 5. ((2S,4R)-4-Amino-1-tritylpyrrolidin-2-yl)methanol

To a stirred suspension of LiAlH₄ (2.20 g, 58.0 mmol) in THE (80 mL) at 0° C. was added a solution of a solution of methyl (2S,4R)-4-azido-1-trityl-pyrrolidine-2-carboxylate (8.00 g, 19.4 mmol) in THE (10 mL). After the addition was completed, the reaction mixture was stirred at 0° C. for 1 h. The reaction was quenched with water (2.5 mL), 15% NaOH solution (2.5 mL) and water (6 mL). After stirring for 0.5 h, the mixture was dried over Na₂SO₄, filtered and concentrated to give crude product [(2S,4R)-4-amino-1-trityl-pyrrolidin-2-yl]methanol as yellow oil (7.00 g), which was applied to the next step without purification.

### Step 6. tert-Butyl ((3R,5S)-5-(hydroxymethyl)-1-tritylpyrrolidin-3-yl)carbamate

To a solution of [(2S,4R)-4-amino-1-trityl-pyrrolidin-2-yl]methanol (7.00 g, 19.5 mmol) in 1,4-dioxane (70 mL) was added TEA (3.90 g, 39.0 mmol) and (Boc)₂O (5.10 g, 23.0 mmol). The mixture was stirred for 2 h at rt, then diluted with water and extracted with EA (3×100 mL). The combined organic layers were washed with water, brine, dried over anhydrous Na₂SO₄ and concentrated. The residue was purified by silica gel column chromatography (PE:EA=20:1) to give tert-butyl N-[(3R,5S)-5-(hydroxymethyl)-1-trityl-pyrrolidin-3-yl]carbamate (8.00 g, 89.3% yield) as colorless oil. MS m/z 459.3 [M+H]⁺, ¹H NMR (400 MHz, CDCl₃) δ 7.67-7.50 (m, 6H), 7.33-7.23 (m, 7H), 7.18 (t, J=7.3 Hz, 3H), 4.27-4.18 (m, 1H), 3.77-3.64 (m, 2H), 3.58-3.51 (m, 1H), 3.50-3.32 (m, 2H), 2.60-2.48 (m, 1H), 2.03-1.94 (m, 1H), 1.90-1.81 (m, 1H), 1.36 (s, 9H).

### Step 7. tert-Butyl ((3R,5R)-5-fluoro-1-tritylpiperidin-3-yl)carbamate

DAST (2.80 mL, 21.0 mmol) was added dropwise to a stirred solution of tert-butyl N-[(3R,5S)-5-(hydroxymethyl)-1-trityl-pyrrolidin-3-yl]carbamate (6.80 g, 15.0 mmol) in THE (70 mL) at 0° C. The mixture was stirred for 1 h at 0° C. and 1 h at RT, then cooled to 0° C. again. A saturated aqueous solution of Na₂CO₃ was added to adjust pH to 12. The phases were separated and the aqueous phase was extracted with EtOAc (2×40 mL). The organic extracts were combined, dried over Na₂SO₄, filtered, and concentrated under reduced pressure. The crude material was purified by flash chromatography (silica gel, PE/EtOAc=20/1) to afford tert-butyl N-[(3R,5R)-5-fluoro-1-trityl-3-piperidyl]carbamate (3.90 g, 57% yield) as a white solid. MS m z 483.3 [M+Na]⁺.

### Step 8. tert-Butyl ((3R,5R)-5-fluoropiperidin-3-yl)carbamate

To a solution of tert-butyl N-[(3R,5R)-5-fluoro-1-trityl-3-piperidyl]carbamate (3.90 g, 8.50 mmol) in MeOH (80 mL) was added AcOH (8 mL, 139.6 mmol). The mixture was stirred for 2 h at 80° C., then concentrated under reduced pressure. The residue was diluted with EA (20 mL) and H₂O (20 mL). The mixture was acidified with aqueous HCl till pH=2-3 and extracted with EA (10 mL×2). The aqueous layers were basified with aqueous K₂CO₃ till pH=9-10 and extracted with DCM (50 mL×3). The DCM layers were combined and dried over Na₂SO₄, filtered and concentrated under reduced pressure to provide tert-butyl N-[(3R,5R)-5-fluoro-3-piperidyl]carbamate (1.60 g, 87% yield) as a white solid. MS m/z 219.1 [M+H]⁺, ¹H NMR (400 MHz, DMSO-d₆) δ 6.61 (s, 1H), 4.77 (d, J=48.5 Hz, 1H), 3.60 (br s, 1H), 2.96-2.82 (m, 2H), 2.77-2.62 (m, 1H), 2.43-2.33 (m, 1H), 2.09-1.97 (m, 1H), 1.78-1.55 (m, 1H), 1.39 (s, 9H)

### Step 9. tert-Butyl ((3R,5R)-5-fluoro-1-methylpiperidin-3-yl)carbamate

To a solution of tert-butyl N-[(3R,5R)-5-fluoro-3-piperidyl]carbamate (1.60 g, 7.30 mmol) in DCE (20 mL) was added formic acid (1.20 g, 26.0 mmol,) and (HCHO)n (0.75 g, 25 mmol) and stirred for 1 h at rt. To the mixture was added NaBH(OAc)₃ (5.4 g, 25.0 mmol) and stirred for 16 h at rt. The reaction mixture was quenched with water, basified with aqueous K₂CO₃ solution and extracted with DCM. The organic phase was combined and washed with brine. The organic phase was dried over Na₂SO₄ and concentrated in vacuum. The residue was purified by silica gel chromatography (PE/EA=3/1) to afford tert-butyl N-[(3R,5R)-5-fluoro-1-methyl-3-piperidyl]carbamate (1.00 g, 59.0% yield) as a white solid. MS m/z 233.1 [M+H]⁺,

### Step 10. (3R,5R)-5-Fluoro-1-methylpiperidin-3-amine hydrogen chloride

To a solution of tert-butyl N-[(3R,5R)-5-fluoro-1-methyl-3-piperidyl]carbamate (1.00 g, 4.30 mmol) in EA (10 mL) was added HCl in 1,4-dioxane (14 mL, 56 mmol, 4 mol/L). The resulting mixture was stirred at room temperature for 2 h, then filtered and the solid was wash with EA (2 mL) and dried under vacuum to give (3R,5R)-5-fluoro-1-methyl-piperidin-3-amine hydrogen chloride (750 mg, 75% yield) as a white solid. MS m/z 133.1 [M+H]⁺, ¹H NMR (400 MHz, DMSO-d₆) δ 11.18 (d, J=3.6 Hz, 1H), 8.94 (s, 3H), 5.25 (d, J=44.4 Hz, 1H), 3.72-3.66 (m, 2H), 3.56-3.50 (m, 1H), 3.41-3.27 (m, 1H), 3.13 (t, J=11.2 Hz, 1H), 2.85 (s, 3H), 2.47-2.41 (m, 1H), 2.01-1.83 (m, 1H).

### Intermediate 3g: (3R,5R)-5-Fluoro-1-ethylpiperidin-3-amine hydrochloride

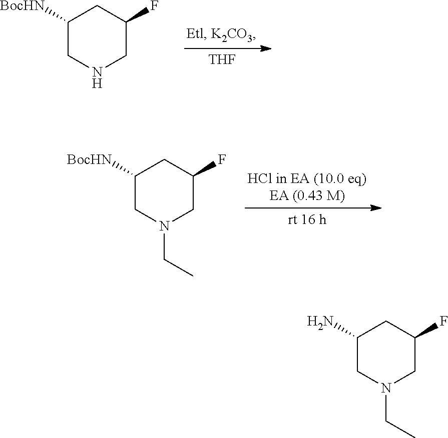

The starting material, tert-butyl ((3R,5R)-5-fluoropiperidin-3-yl) carbamate, was prepared as the procedure of Intermediate 3f step 1 to 8.

The title compound was prepared in analogous manner according to the procedure of Intermediate 3b, using tert-butyl ((3R,5R)-5-fluoropiperidin-3-yl)carbamate in place of tert-butyl (R)-piperidin-3-ylcarbamate in step 1. MS m/z 147.2 [M+H]⁺; ¹H NMR (400 MHz, DMSO-d₆) δ 11.02 (s, 1H), 8.94 (s, 3H), 5.27 (d, J=44.8 Hz, 1H), 3.74-3.62 (m, 3H), 3.32-3.22 (m, 3H), 3.09-3.05 (m, 1H), 2.48-2.43 (m, 1H), 2.06-1.89 (m, 1H), 1.28-1.25 (t, J=14.0 Hz, 3H).

### Example 1

### Preparation of Compound 1

### (1s,3s)-3-((1-(4-Methoxy-2-(2,2,2-trifluoroethyl)phenyl)pyrrolo[1,2-d][1,2,4]triazin-4-yl)amino)-1-methylcyclobutan-1-ol

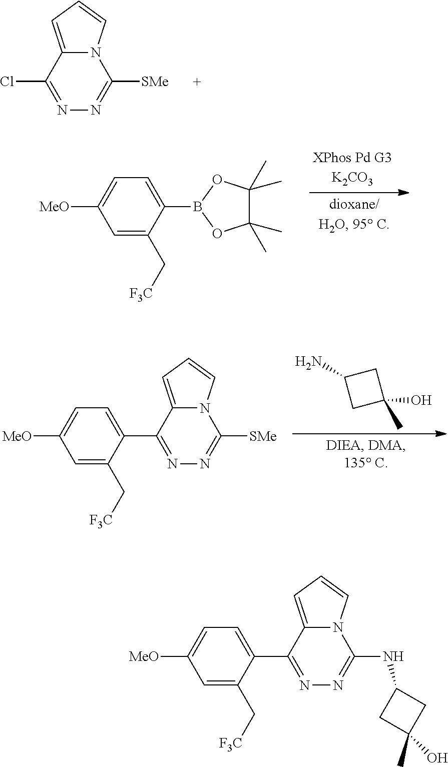

### Step 1: 1-[4-Methoxy-2-(2,2,2-trifluoroethyl)phenyl]-4-methylsulfanyl-pyrrolo[1,2-d][1,2,4]triazine

1-Chloro-4-methylsulfanyl-pyrrolo[1,2-d][1,2,4]triazine (Intermediate 1c, 0.380 g, 1.90 mmol), 2-[4-methoxy-2-(2,2,2-trifluoroethyl)phenyl]-4,4,5,5-tetramethyl-1,3,2-dioxa-borolane (Intermediate 2a, 0.842 g, 2.66 mmol) and XPhos Pd G3 (0.164 g, 0.190 mmol) were added to a vial and evacuated and refilled with Ar, to which was added dioxane (9.5 mL) and K₂CO₃ (2 M, 2.9 mL, 5.71 mmol). The mixture was sparged with Ar for 5 min, then heated to 95° C. for 3 h. After cooled to rt, the mixture was diluted with EtOAc and filtered through Celite. The filtrate was washed with brine, dried (MgSO₄), filtered, and concentrated. Purification by chromatography on SiO₂ (EtOAc:hexanes, 0 to 30%) gave a pink solid (0.329 g, 49%). MS m z 354.4 [M+H]⁺.

### Step 2: (1s,3s)-3-((1-(4-Methoxy-2-(2,2,2-trifluoroethyl)phenyl)pyrrolo[1,2-d][1,2,4]triazin-4-yl)amino)-1-methylcyclobutan-1-ol

1-[4-Methoxy-2-(2,2,2-trifluoroethyl)phenyl]-4-methylsulfanyl-pyrrolo[1,2-d][1,2,4]triazine (0.070 g, 0.18 mmol) and 3-amino-1-methyl-cyclobutanol hydrochloride (0.030 g, 0.22 mmol) in DMA (0.10 mL) and iPr₂NEt (0.10 mL, 0.57 mmol) was heated to 135° C. for 4 h. The reaction was cooled to rt and diluted with DCM/iPrOH (9:1). The solution was washed with brine, dried (Na₂SO₄), filtered, and concentrated. Purification by chromatography on SiO₂ (MeOH:DCM, 0 to 10%) followed by reverse phase chromatography (0.1% formic acid in MeCN:0.100 formic acid in H₂O, 5 to 1000%) gave a white solid (0.010 g, 280%). MS m/z 407.3 [M+H]+; 1H NMR (500 MHz, Methanol-d₄) δ 7.83 (d, J=1.8 Hz, 1H), 7.47 (d, J=8.5 Hz, 1H), 7.10 (s, 1H), 7.09-7.04 (m, 1H), 6.96 (t, J=3.4 Hz, 1H), 6.53 (d, J=3.8 Hz, 1H), 4.31-4.21 (m, 1H), 3.89 (s, 3H), 3.69 (q, J=11.14 Hz, 2H), 2.69-2.62 (m, 2H), 2.31-2.22 (m, 2H), 1.44 (s, 3H). 2H not observed (NH and OH).

The compounds below were prepared according to the procedure of Example 1 by substituting the appropriate starting materials, reagents and reaction conditions.

| Compound# | Spectral Data |
|---|---|
| 5 | MS m/z 492.3 [M + H]+; 1H NMR (400 MHz, Methanol-d₄) δ 7.81-7.74 |
| | (m, 1H), 7.73-7.66 (m, 2H), 7.62-7.54 (m, 1H), 7.15-6.73 (m, 2H), 5.14- |
| | 4.96 (m, 1H), 4.84-4.71 (m, 1H), 3.51-3.39 (m, 1H), 3.26-3.17 (m, |
| | 1H), 2.78-2.65 (m, 2H), 2.64-2.27 (m, 3H), 2.06-1.83 (m, 1H), 1.25- |
| | 1.10 (m, 3H). 1H not observed (NH). |
| 6 | MS m/z 514.3 [M + H]+; 1H NMR (400 MHz, Methanol-d₄) δ 8.29-8.09 (m, |
| | 2H), 7.99-7.77 (m, 2H), 7.02 (s, 1H), 6.49 (s, 1H), 4.73-4.52 (m, 1H), |
| | 4.19-4.00 (m, 2H), 3.97-3.81 (m, 1H), 3.60-3.38 (m, 4H), 3.14-2.91 |
| | (m, 2H), 2.43-1.67 (m, 8H). 1H not observed (NH). |
| 7 | MS m/z 458.9 [M + H]+; 1H NMR (400 MHz, Methanol-d₄) δ 8.30-8.05 (m, |
| | 2H), 7.96-7.71 (m, 2H), 7.10 (s, 1H), 6.62 (s, 1H), 4.32-4.16 (m, 1H), |
| | 3.97-3.84 (m, 1H), 3.81-3.68 (m, 1H), 3.54-3.39 (m, 1H), 3.27-3.11 (m, |
| | 1H), 2.98 (s, 6H), 2.46-2.26 (m, 1H), 2.17-1.82 (m, 3H). NH not |
| | observed. |
| 8 | MS m/z 422.2 [M + H]+; 1H NMR (400 MHz, Methanol-d₄) δ 7.83 (dd, J = |
| | 3.0, 1.2 Hz, 1H), 7.55 (d, J = 8.6 Hz, 1H), 7.09 (dd, J = 8.6, 2.5 Hz, 1H), |
| | 7.01 (dq, J = 2.8, 1.4 Hz, 1H), 6.95 (dd, J = 4.0, 2.9 Hz, 1H), 6.54 (dd, J = |
| | 3.9, 1.2 Hz, 1H), 4.48 (tt, J = 8.9, 4.0 Hz, 1H), 3.90 (s, 3H), 3.27-3.13 (m, |
| | 1H), 2.87-2.68 (m, 1H), 2.50-2.27 (m, 5H), 2.10 (d, J = 9.8 Hz, 1H), 1.90 |
| | (dp, J = 13.2, 4.8 Hz, 1H), 1.78 (dtt, J = 13.7, 6.3, 3.2 Hz, 1H), 1.68 (dd, J = |
| | 24.3, 13.5 Hz, 1H). NH not observed. |
| 9 | MS m/z 409.3 [M + H]+; 1H NMR (400 MHz, Methanol-d₄) δ 7.80 (dd, J = |
| | 2.9, 1.2 Hz, 1H), 7.55 (d, J = 8.6 Hz, 1H), 7.09 (dd, J = 8.6, 2.5 Hz, 1H), |
| | 7.01 (dq, J = 2.9, 1.5 Hz, 1H), 6.94 (dd, J = 3.9, 2.9 Hz, 1H), 6.53 (dd, J = |
| | 4.0, 1.2 Hz, 1H), 4.35-4.18 (m, 1H), 3.90 (s, 3H), 2.65-2.58 (m, 2H), |
| | 2.34-2.18 (m, 2H), 1.44 (s, 3H). NH and OH not observed. |
| 10 | MS m/z 484.4 [M − H]−; 1H NMR (400 MHz, Methanol-d₄) δ = 8.14 (s, 1H), |
| | 8.08-8.06 (m, 1H), 7.84 (s, 1H), 7.79 (d, J = 8.0 Hz, 1H), 6.98-6.92 (m, |
| | 1H), 6.45-6.39 (m, 1H), 4.64-4.53 (m, 1H), 3.79 (d, J = 10.0 Hz, 1H), |
| | 3.42 (d, J = 10.9 Hz, 1H), 3.04-2.87 (m, 4H), 2.28-2.05 (m, 3H), 2.04- |
| | 1.77 (m, 2H), 1.03-0.98 (m, 6H); NH not observed. |
| 11 | MS m/z 406.3, 408.2 [M + H]+; 1H NMR (400 MHz, Methanol-d₄) δ 7.82 (s, |
| | 1H), 7.72-7.58 (m, 3H), 6.93 (m, 1H), 6.44 (s, 1H), 4.47 (m, 1H), 3.47 (m, |
| | 1H), 3.06 (m, 1H), 2.75-2.56 (m, 5H), 2.33 (s, 3H), 2.16 (m, 1H), 2.03 (m, |
| | 1H), 1.83 (m, 1H), 1.71 (m, 1H). |
| 12 | MS m/z 418.3 [M + H]+; 1H NMR (400 MHz, DMSO-d₆)δ 8.20 (s, 1H, |
| | formate CH), 7.78-7.69 (m, 1H), 7.45 (d, J = 8.5 Hz, 1H), 7.20 (s, 1H), |
| | 7.03-7.00 (m, 1H), 6.96 (m, 1H), 6.91-6.84 (m, 1H), 6.23 (s, 1H), 4.36- |
| | 4.20 (m, 1H), 3.85 (s, 3H), 3.05 (m, 1H), 2.71 (m, 1H), 2.23 (m, 6H), 2.05- |
| | 1.86 (m, 3H), 1.75 (m, 1H), 1.70-1.51 (m, 1H), 1.51-1.23 (m, 1H). |
| 13 | MS m/z 422.3, 424.3 [M + H]+; 1H NMR (400 MHz, DMSO-d₆) δ 8.17 (s, |
| | 1H, formate CH), 7.80-7.72 (m, 1H), 7.62-7.54 (m, 1H), 7.51-7.44 (m, |
| | 2H), 7.16-7.07 (m, 1H), 6.27 (s, 1H), 4.27 (br, s, 1H), 3.45 (br, 1H), 3.07 |
| | (d, J = 10.0 Hz, 1H), 2.72 (d, J = 10.8 Hz, 1H), 2.24 (m, 6H), 2.04-1.89 |
| | (m, 3H), 1.84-1.71 (m, 1H), 1.59 (m, 1H), 1.51-1.36 (m, 1H). |
| 14 | MS m/z 412.4 [M + H]+; 1H NMR (400 MHz, DMSO-d₆) δ 8.14 (s, 1H, |
| | formate CH), 7.83-7.74 (m, 1H), 7.61-7.49 (m, 2H), 7.33 (d, J = 8.3 Hz, |
| | 1H), 7.16-7.05 (m, 2H), 6.35 (s, 1H), 4.30-4.28 (m, 1H), 3.10-3.08 (m, |
| | 1H), 2.77-2.74 (m, 1H), 2.26 (s, 6H), 2.11 (m, 1H), 1.99 (m, 3H), 1.79- |
| | 1.76 (m, 1H), 1.62 (m, 1H), 1.51-1.44 (m, 1H), 1.07-1.04 (m, 2H), 0.79 |
| | (m, 2H). |
| 15 | MS m/z 490.2 [M + H]+; 1H NMR (400 MHz, Methanol-d₄) δ 7.80-7.74 |
| | (m, 2H), 7.73-7.68 (m, 1H), 7.61-7.57 (m, 1H), 7.13-6.72 (m, 2H), 4.54- |
| | 4.40 (m, 1H), 3.81-3.65 (m, 2H), 3.22-3.09 (m, 1H), 2.81-2.70 (m, |
| | 1H), 2.69-2.55 (m, 2H), 2.51-2.35 (m, 2H), 2.10-1.97 (m, 1H), 1.93- |
| | 1.82 (m, 1H), 1.81-1.62 (m, 2H). 2H not observed (NH and OH). |
| 16 | MS m/z 421.2 [M + H]+; 1H NMR (400 MHz, Methanol-d₄) δ 7.80-7.74 |
| | (m, 1H), 7.74-7.69 (m, 1H), 7.69-7.65 (m, 1H), 7.60-7.57 (m, 1H), 7.13- |
| | 6.72 (m, 2H), 4.25-4.13 (m, 1H), 3.72 (dd, 1H), 3.53 (dd, 1H), 1.27 (d, |
| | 3H). 2H not observed (NH and OH). |
| 17 | MS m/z 438.3 [M + H]+; 1H NMR (400 MHz, Methanol-d₄) δ 7.68 (t, J = 3.6 |
| | Hz, 1H), 7.46 (d, J = 8.5 Hz, 1H), 7.34 (d, J = 2.5 Hz, 1H), 7.28 (dd, J = 8.4, |
| | 2.5 Hz, 1H), 6.75 (d, J = 3.1 Hz, 1H), 4.54-4.37 (m, 1H), 3.93 (s, 3H), 3.31- |
| | 3.18 (m, 1H), 2.96-2.80 (m, 1H), 2.71-2.54 (m, 2H), 2.44-2.27 (m, |
| | 2H), 2.21-2.05 (m, 1H), 2.00-1.84 (m, 1H), 1.83-1.71 (m, 1H), 1.70- |
| | 1.55 (m, 1H), 1.18 (t, J = 7.2 Hz, 3H). 1H not observed (NH). |
| 18 | MS m/z 454.2 [M + H]+; 1H NMR (400 MHz, Methanol-d₄) δ 7.70 (t, J = 3.7 |
| | Hz, 1H), 7.46 (d, J = 8.5 Hz, 1H), 7.35 (d, J = 2.5 Hz, 1H), 7.29 (dd, J = 8.5, |
| | 2.6 Hz, 1H), 6.76 (d, J = 3.3 Hz, 1H), 4.58-4.41 (m, 1H), 3.93 (s, 3H), 3.84- |
| | 3.74 (m, 2H), 3.54-3.39 (m, 1H), 3.16-3.00 (m, 1H), 2.97-2.82 (m, |
| | 2H), 2.78-2.58 (m, 2H), 2.19-2.06 (m, 1H), 2.04-1.92 (m, 1H), 1.92- |
| | 1.66 (m, 2H). 2H not observed (NH and OH). |
| 19 | MS m/z [M + H]+ 394.1; 1H NMR (400 MHz, Methanol-d₄) δ 8.52 (br s, 1H, |
| | formic acid), 7.89-7.81 (m, 2H), 7.72-7.763 (m, 2H), 7.03-6.99 (m, |
| | 1H), 6.68 (s, 1H), 4.58-4.45 (m, 1H), 3.43-3.33 (m, 1H), 2.99-2.85 (m, |
| | 1H), 2.59-2.42 (m, 5H), 2.21-2.06 (m, 1H), 2.06-1.90 (m, 1H), 1.90- |
| | 1.75 (m, 1H), 1.75-1.64 (m, 1H). 1H not observed (NH). |
| 20 | MS m/z [M + H]+ 408.3; 1H NMR (400 MHz, Methanol-d₄) δ 7.89-7.83 |
| | (m, 2H), 7.72-7.66 (m, 2H), 7.02 (t, J = 3.3 Hz, 1H), 6.69 (s, 1H), 4.58- |
| | 4.58 (m, 1H), 3.66-3.53 (m, 1H), 3.25-3.13 (m, 1H), 2.96-2.86 (m, 2H), |
| | 2.77-2.57 (m, 2H), 2.26-2.14 (m, 1H), 2.11-1.99 (m, 1H), 1.95-1.71 |
| | (m, 2H), 1.28 (t, J = 7.3 Hz, 3H). 1H not observed (NH). |
| 21 | MS m/z [M + H]+ 424.2; 1H NMR (400 MHz, Methanol-d₄) δ 7.90-7.83 |
| | (m, 2H), 7.73-7.66 (m, 2H), 7.03 (t, J = 3.4 Hz, 1H), 6.70 (s, 1H), 4.63- |
| | 4.54 (m, 1H), 3.84 (t, J = 5.4 Hz, 2H), 3.75-3.63 (m, 1H), 3.28-3.23 (m, |
| | 1H), 3.05 (q, J = 5.1 Hz, 2H), 2.91-2.76 (m, 2H), 2.23-2.14 (m, 1H), 2.13- |
| | 2.01 (m, 1H), 2.00-1.75 (m, 2H). 2H not observed (NH and OH). |
| 22 | MS m/z 486.3 [M + H]+; 1H NMR (400 MHz, Methanol-d₄) δ 7.84-7.93 (m, |
| | 1 H) 7.73-7.84 (m, 2 H) 7.69 (s, 1 H) 6.72-7.15 (m, 2 H) 6.62 (d, J = 3.63 |
| | Hz, 1 H) 4.53-4.75 (m, 1 H) 3.67-4.00 (m, 3 H) 3.43-3.58 (m, 2 H) 2.99- |
| | 3.26 (m, 2 H) 2.11-2.36 (m, 2 H) 1.76-2.10 (m, 2 H) 1.37 (dd, J = 6.44, |
| | 3.69 Hz, 3 H); 2H not observed (NH and OH). |
| 23 | MS m/z 386.4 [M + H]+; 1H NMR(400 MHz, Methanol-d₄) δ 8.67-8.44 (s, |
| | formate CH), 7.63 (d, J = 9.6 Hz, 2H), 7.48 (m, 2H), 7.06-6.62 (m, 1H), |
| | 6.38 (s, 1H), 4.56-4.37 (m, 1H), 3.00-2.83 (m, 1H), 2.60-2.43 (m, 8H), |
| | 2.32 (s, 4H), 2.21-2.06 (m, 1H), 2.04-1.89 (m, 1H), 1.87-1.77 (m, 1H), |
| | 1.69 (m, 1H). NH peak was not observed. |
| 24 | MS m/z 428.5 [M + H]+; 1H NMR (400 MHz, Methanol-d₄) δ 8.63-8.39 (s, |
| | 1H, formate CH), 7.57 (s, 1H), 7.40 (d, J = 7.9 Hz, 1H), 7.09 (d, J = 7.9 Hz, |
| | 1H), 7.04 (s, 1H), 6.94-6.53 (m, 1H), 6.36 (s, 1H), 4.55-4.38 (m, 1H), |
| | 3.39 (m, 1H), 3.06-2.90 (m, 1H), 2.57 (br, s, 5H), 2.32 (s, 3H), 2.20-2.07 |
| | (m, 1H), 2.07-1.92 (m, 2H), 1.89-1.76 (m, 1H), 1.74-1.63 (m, 1H), 1.14- |
| | 1.01 (m, 2H), 0.85-0.72 (m, 2H). NH peak not observed. |
| 25 | MS m/z 402.4 [M + H]+; 1H NMR (400 MHz, Methanol-d₄) δ 8.53 (s, 1H, |
| | formate CH), 7.67-7.53 (m, 1H), 7.45-7.36 (m, 1H), 7.27-7.10 (m, 2H), |
| | 6.73 (m, 1H), 6.36 (s, 1H), 4.54-4.40 (m, 1H), 3.51-3.38 (m, 1H), 3.09- |
| | 2.93 (m, 1H), 2.66-2.54 (m, 5H), 2.46 (s, 3H), 2.32 (s, 3H), 2.13 (m, 1H), |
| | 2.05-1.94 (m, 1H), 1.85 (m, 1H), 1.72 (m, 1H). NH peak was not |
| | observed. |
| 26 | MS m/z 352.5 [M + H]+; 1H NMR (400 MHz, Methanol-d₄) δ 7.85 (s, 1H), |
| | 7.29 (d, J = 8.4 Hz, 1H), 6.96 (t, J = 3.2 Hz, 1H), 6.93 (d, J = 7.2 Hz, 1H), |
| | 6.88 (dd, J = 2.4 Hz, 8.4 Hz, 1H), 6.49 (d, J = 3.2 Hz, 1H), 4.54 (s, 1H), |
| | 3.85(s, 3H), 3.66 (s, 1H), 3.30-3.29 (m, 1H), 2.92 (s, 1H), 2.79 (s, 3H), |
| | 2.21 (s, 3H), 2.13 (s, 1H), 2.10-2.11 (m, 2H), 1.84-1.93 (m, 2H). NH peak |
| | not observed. |
| 27 | MS m/z 378.3 [M + H]+; 1H NMR (400 MHz, Methanol-d₄) δ 7.84 (d, J = |
| | 1.6 Hz, 1H), 7.26 (d, J = 8.8 Hz, 1H), 6.95 (t, J = 4.0 Hz, 1H), 6.86 (dd, J = |
| | 2.4 Hz, 8.4 Hz, 1H), 6.57 (d, J = 2.8 Hz, 1H), 6.53 (d, J = 4.0 Hz, 1H), 4.54 |
| | (s, 1H), 3.82 (s, 3H), 3.57 (s, 1H), 3.12-3.16 (m, 1H), 2.79-2.85 (m, 2H), |
| | 2.73 (s, 3H), 2.18 (dd, J = 3.2 Hz, 9.2 Hz, 1H), 2.07 (dd, J = 4.4 Hz, 9.6 Hz, |
| | 1H), 1.82-1.93 (m, 1H), 1.74-1.81 (m, 2H), 0.70-0.76 (m, 2H), 0.62- |
| | 0.68 (m, 2H). NH not observed |
| 28 | MS m/z 462.2 [M + H]+; 1H NMR (400 MHz, Methanol-d₄) δ 8.15 (s, 1H), |
| | 8.10 (d, J = 8.0 Hz, 1H), 7.81 (d, J = 7.9 Hz, 1H), 7.74 (t, J = 3.7 Hz, 1H), |
| | 6.80 (d, J = 3.0 Hz, 1H), 4.55-4.37 (m, 1H), 3.22-3.07 (m, 1H), 2.82- |
| | 2.66 (m, 1H), 2.45-2.19 (m, 5H), 2.15-2.00 (m, 1H), 1.97-1.82 (m, 1H), |
| | 1.82-1.69 (m, 1H), 1.68-1.52 (m, 1H). 1H not observed (NH). |
| 29 | MS m/z 476.2 [M + H]+; 1H NMR (400 MHz, Methanol-d₄) δ 8.15 (s, 1H), |
| | 8.10 (d, J = 8.0 Hz, 1H), 7.81 (d, J = 8.0 Hz, 1H), 7.74 (t, J = 3.7 Hz, 1H), |
| | 6.80 (d, J = 3.2 Hz, 1H), 4.55-4.39 (m, 1H), 3.28-3.13 (m, 1H), 2.93- |
| | 2.73 (m, 1H), 2.68-2.47 (m, 2H), 2.42-2.22 (m, 2H), 2.18-2.04 (m, 1H), |
| | 1.97-1.84 (m, 1H), 1.82-1.70 (m, 1H), 1.69-1.53 (m, 1H), 1.16 (t, J = |
| | 7.2 Hz, 3H). 1H not observed (NH). |
| 30 | MS m/z 492.2 [M + H]+; 1H NMR (400 MHz, Methanol-d₄) δ 8.16 (s, 1H), |
| | 8.11 (d, J = 8.3 Hz, 1H), 7.82 (d, J = 8.0 Hz, 1H), 7.77 (t, J = 3.7 Hz, 1H), |
| | 6.81 (d, J = 3.3 Hz, 1H), 4.60-4.43 (m, 1H), 3.90-3.72 (m, 2H), 3.58- |
| | 3.41 (m, 1H), 3.18-3.03 (m, 1H), 3.00-2.82 (m, 2H), 2.80-2.55 (m, 2H), |
| | 2.22-2.07 (m, 1H), 2.06-1.94 (m, 1H), 1.92-1.69 (m, 2H). 2H not |
| | observed (NH and OH). |
| 31 | MS m/z 480.2 [M + H]+; 1H NMR (400 MHz, Methanol-d₄) δ 8.15 (s, 1H), |
| | 8.10 (d, J = 7.9 Hz, 1H), 7.81 (d, J = 7.9 Hz, 1H), 7.71 (t, J = 3.6 Hz, 1H), |
| | 6.80 (d, J = 3.1 Hz, 1H), 5.10-4.91 (m, 1H), 4.83-4.69 (m, 1H), 3.29- |
| | 3.23 (m, 1H), 3.11-2.97 (m, 1H), 2.53-2.33 (m, 5H), 2.31-2.22 (m, 1H), |
| | 2.00-1.78 (m, 1H). NH not observed. |
| 32 | MS m/z 494.2 [M + H]+; 1H NMR (400 MHz, Methanol-d₄) δ 8.15 (s, 1H), |
| | 8.10 (d, J = 8.3 Hz, 1H), 7.82 (d, J = 7.9 Hz, 1H), 7.71 (t, J = 3.6 Hz, 1H), |
| | 6.80 (d, J = 3.1 Hz, 1H), 5.10-4.92 (m, 1H), 4.82-4.71 (m, 1H), 3.40- |
| | 3.32 (m, 1H), 3.19-3.05 (m, 1H), 2.69-2.57 (m, 2H), 2.53-2.37 (m, 2H), |
| | 2.35-2.23 (m, 1H), 2.03-1.81 (m, 1H), 1.16 (t, J = 7.2 Hz, 3H). NH not |
| | observed. |
| 33 | MS m/z 388.3 [M + H]+; 1H NMR (400 MHz, Methanol-d₄) δ 7.85 (s, 1H), |
| | 7.45 (d, J = 5.9 Hz, 1H), 7.33-6.73 (m, 4H), 6.58 (s, 1H), 4.52 (s, 1H), 3.90- |
| | 3.66 (m, 1H), 3.47-3.35 (m, 1H), 3.18-2.97 (m, 2H), 2.88 (s, 3H), 2.48 |
| | (s, 3H), 2.33-2.09 (m, 2H), 2.05-1.76 (m, 2H); NH not observed. |
| 34 | MS m/z 402.3 [M + H]+; 1H NMR (400 MHz, Methanol-d₄) δ 8.49 (br, 1H), |
| | 7.85 (s, 1H), 7.45 (d, J = 7.8 Hz, 1H), 7.31-6.75 (m, 4H), 6.58 (d, J = 4.1 |
| | Hz, 1H), 4.69-4.52 (m, 1H), 3.99-3.79 (m, 1H), 3.56-3.41 (m, 1H), 3.29- |
| | 3.17 (m, 2H), 3.17-2.91 (m, 2H), 2.48 (s, 3H), 2.37-2.11 (m, 2H), 2.06- |
| | 1.85 (m, 2H), 1.38 (t, J = 7.2 Hz, 3H). NH not observed. |
| 35 | MS m/z 428.2 [M + H]+; 1H NMR (400 MHz, Methanol-d₄) δ 8.60 (d, J = |
| | 1.2 Hz, 1H), 7.90 (dd, J = 55.6, 7.6 Hz, 2H), 7.83 (s, 1H), 7.41-7.38 (m, |
| | 2H), 7.10 (t, J = 72.4 Hz, 1H), 4.64-4.57 (m, 1H), 3.77-3.73 (m, 1H), |
| | 3.46-3.84 (m, 1H), 3.28-3.22 (m, 1H), 3.17-3.11 (m, 1H). 2.33-2.30 |
| | (m, 1H), 2.21-2.16 (m, 1H), 2.08-1.94 (m, 1H). NH not observed |
| 36 | MS m/z 406.3 [M + H]+; ¹HNMR (400 MHz, Methanol-d₄) δ 7.82 (d, J = 2.1 |
| | Hz, 1H), 7.50 (d, J = 7.8 Hz, 1H), 7.35-7.29 (m, 2H), 6.97-6.92 (m, 1H), |
| | 6.51 (d, J = 3.8 Hz, 1H), 4.47 (s, 1H), 3.18-3.07 (m, 1H), 2.74-2.65 (m, |
| | 1H), 2.48 (s, 3H), 2.34 (s, 3H), 2.32-2.19 (m, 2H), 2.12-2.04 (m, 1H), |
| | 1.90-1.83 (m, 1H), 1.79-1.71 (m, 1H), 1.61 (dt, J = 11.2, 6.7 Hz, 1H). |
| | NH not observed. |
| 37 | MS m/z 432.2 [M + H]+; ¹HNMR (400 MHz, Methanol-d₄) δ 8.53 (s, 1H), |
| | 7.82 (s, 1H), 7.49 (d, J = 7.9 Hz, 1H), 7.25-7.16 (m, 2H), 6.99-6.91 (m, |
| | 1H), 6.55 (d, J = 3.5 Hz, 1H), 4.56-4.45 (m, 1H), 3.51-3.39(m, 1H), 3.07- |
| | 2.95 (m, 1H), 2.72-2.60 (m, 2H), 2.58 (s, 3H), 2.23-2.12 (m, 1H), 2.09- |
| | 1.96 (m, 2H), 1.88-1.69 (m, 2H), 1.14-1.07 (m, 2H), 0.84-0.76 (m, |
| | 2H). NH not observed. |
| 38 | MS m/z 449.2 [M + H]+; ¹HNMR (400 MHz, Methanol-d₄) δ 8.15 (s, 1H), |
| | 8.10 (d, J = 8.0 Hz, 1H), 7.81 (d, J = 8.0 Hz, 1H), 7.73 (t, J = 3.7 Hz, 1H), |
| | 6.79 (d, J = 3.4 Hz, 1H), 4.29-4.19 (m, 1H), 2.70-2.61 (m, 2H), 2.30- |
| | 2.21 (m, 2H), 1.43 (s, 3H). 2H not observed (NH and OH) |
| 39 | MS m/z 440.3 [M + H]+; 1H NMR (400 MHz, Methanol-d₄) δ 7.72 (t, J = |
| | 3.7 Hz, 1H), 7.53 (d, J = 8.6 Hz, 1H), 7.10 (dd, J = 8.6, 2.4 Hz, 1H), 7.03- |
| | 6.96 (m, 1H), 6.82 (d, J = 3.3 Hz, 1H), 4.58-4.43 (m, 1H), 3.93 (s, 3H), |
| | 3.71-3.53 (m, 1H), 3.26-3.16 (m, 1H), 2.95-2.78 (m, 2H), 2.76 (s, 3H), |
| | 2.25-2.04 (m, 2H), 1.97-1.70 (m, 2H). NH not observed. |
| 40 | MS m/z 458.3 [M + H]+; 1H NMR (400 MHz, Methanol-d₄) δ 7.94-7.87 |
| | (m, 2H), 7.84 (d, J = 8.25 Hz, 1H), 7.76 (d, 9.26 Hz, 1H), 7.01 (dd, J = 3.8, |
| | 3.0 Hz, 1H), 6.57 (dd, J = 3.9, 0.9 Hz, 1H), 4.58-4.49 (m, 1H), 3.83 (q, J = |
| | 10.9 Hz, 2H), 3.62-3.52 (m, 1H), 3.21-3.11 (m, 1H), 2.87-2.73 (m, |
| | 2H), 2.70 (s, 3H), 2.26-2.14 (m, 1H), 2.12-2.01 (m, 1H), 1.97-1.68 (m, |
| | 2H). 1H not observed (NH). |
| 41 | MS m/z 445.2 [M + H]+; 1H NMR (400 MHz, Methanol-d₄) δ 7.92-7.86 (m, |
| | 2H), 7.83 (d, J = 7.88 Hz, 1H), 7.77 (d, J = 8.13 Hz, 1H), 6.99 (t, J = 3.4 |
| | Hz, 1H), 6.55 (d, J = 3.9 Hz, 1H), 4.33-4.23 (m, 1H), 3.85 (q, J = 11.0 Hz, |
| | 2H), 2.71-2.62 (m, 2H), 2.34-2.23 (m, 2H), 1.45 (s, 3H). 2H not |
| | observed (NH and OH) |
| 42 | MS m/z 420.2 [M + H]+; 1HNMR(400 MHz, Methanol-d₄) δ 8.50-8.44 (m, |
| | 1H), 7.84 (d, J = 1.8 Hz, 1H), 7.54 (d, J = 7.8 Hz, 1H), 7.39 (d, J = 7.8 Hz, |
| | 1H), 7.34 (s, 1H), 7.01-6.95 (m, 1H), 6.57 (d, J = 3.7 Hz, 1H), 4.57-4.49 |
| | (m, 1H), 3.67 (tdd, J = 10.2, 6.6, 3.1 Hz, 1H), 3.29-3.24 (m, 1H), 3.00- |
| | 2.85 (m, 2H), 2.82 (d, J = 7.6 Hz, 1H), 2.78 (d, J = 4.8 Hz, 4H), 2.21 (dt, J = |
| | 18.3, 6.6 Hz, 1H), 2.14-2.07 (m, 1H), 1.95-1.79 (m, 2H), 1.32 (t, J = 7.6 |
| | Hz, 3H). NH not observed. |
| 43 | MS m/z 436.3 [M + H]+; 1H NMR (500 MHz, Methanol-d₄) δ 7.84 (d, J = |
| | 2.8 Hz, 1H), 7.55 (d, J = 8.5 Hz, 1H), 7.10 (dd, J = 8.6, 2.4 Hz, 1H), 7.00 |
| | (dd, J = 7.4, 3.7 Hz, 2H), 6.60 (d, J = 3.9 Hz, 1H), 4.56 (tt, J = 9.7, 4.1 Hz, |
| | 1H), 4.16 (q, J = 7.0 Hz, 2H), 3.80 (s, 1H), 3.48-3.36 (m, 1H), 3.05 (d, J = |
| | 12.7 Hz, 2H), 2.90 (s, 3H), 2.28-2.12 (m, 2H), 2.03-1.84 (m, 2H), 1.46 |
| | (t, J = 6.9 Hz, 3H). NH not observed. |
| 44 | MS m/z 458.3 [M + H]+; 1H NMR (500 MHz, Methanol-d₄) δ 7.87 (d, J = |
| | 2.9 Hz, 1H), 7.69 (d, J = 8.5 Hz, 1H), 7.36 (dd, J = 8.4, 2.4 Hz, 1H), 7.32 (t, |
| | J = 1.9 Hz, 1H), 7.04 (t, J = 75 Hz, 1H), 7.02-6.98 (m, 1H), 6.61 (dd, J = |
| | 4.0, 1.1 Hz, 1H), 4.57 (tt, J = 9.2, 3.7 Hz, 1H), 3.86-3.73 (m, 1H), 3.50- |
| | 3.36 (m, 1H), 3.18-2.99 (m, 2H), 2.91 (s, 3H), 2.30-2.12 (m, 2H), 2.01- |
| | 1.81 (m, 2H). NH not observed. |
| 45 | MS m/z 418.3 [M + H]+; 1H NMR (400 MHz, Methanol-d₄) δ 7.80-7.95 (m, |
| | 1 H) 7.38-7.53 (m, 1 H) 7.13-7.30 (m, 2 H) 6.46-7.03 (m, 3 H) 4.39- |
| | 4.57 (m, 1 H) 3.74 (d, J = 5.75 Hz, 2 H) 3.08-3.26 (m, 1 H) 2.70-2.81 (m, 1 |
| | H) 2.54-2.70 (m, 2 H) 2.52-2.33 (m, 5 H) 1.97-2.14 (m, 1 H) 1.61-1.95 |
| | (m, 3 H); 2H not observed (NH and OH). |
| 46 | MS m/z 472.3 [M + H]+; 1H NMR (400 MHz, Methanol-d₄) δ 7.97 (s, 1H), |
| | 7.86-7.74 (m, 2H), 7.73-7.67 (m, 1H), 7.20-6.76 (m, 2H), 6.76-6.66 |
| | (m, 1H), 4.65-4.39 (m, 1H), 4.26-3.91 (m, 2H), 3.81-3.57 (m, 1H), 3.32- |
| | 3.24 (m, 2H), 3.23-2.96 (m, 1H), 2.52-2.29 (m, 1H), 2.13-1.84 (m, |
| | 1H), 1.50-1.30 (m, 3H); 2H not observed (NH and OH). CH obscured by |
| | the signal of Methanol-d₄. |
| 47 | MS m/z 416.2, 418.2 [M + H]+; 1H NMR (400 MHz, Methanol-d₄) δ 8.38 (s, |
| | 1H), 7.62 (d, J = 8.8 Hz, 1H), 7.48 (d, J = 2.4 Hz, 1H), 7.37 (s, 1H), 7.29 (d, |
| | J = 4.4 Hz, 1H), 7.22 (dd, J = 2.0 Hz, J = 8.4 Hz, 1H), 4.67-4.54 (m, 1H), |
| | 4.00-3.94 (m, 1H), 3.94 (s, 3H), 3.60-3.58 (m, 1H), 3.06-2.92 (m, 2H), |
| | 2.97 (s, 3H), 2.35-2.33 (m, 1H), 2.19-2.16 (m, 1H), 2.03-1.84 (m, 2H). |
| | NH not observed. |
| 48 | MS m/z 472.2 [M + H]+; 1H NMR (400 MHz, Methanol-d₄) δ 7.99-7.87 (m, |
| | 1H), 7.80 (d, J = 8.0 Hz, 1H), 7.77-7.71 (m, 1H), 7.67 (s, 1H), 7.13-6.69 |
| | (m, 2H), 6.58 (d, J = 3.8 Hz, 1H), 4.71-4.58 (m, 1H), 4.34-4.20 (m, 1H), |
| | 3.04-2.47 (m, 6H), 2.06-1.85 (m, 2H), 1.18 (t, J = 7.2 Hz, 3H); 2H not |
| | observed (NH and OH). |
| 49 | MS m/z 456.1 [M + H]+; 1HNMR (400 MHz, Methanol-d₄) δ 7.85 (d, J = |
| | 2.0 Hz, 1H), 7.59 (s, 1H), 7.45 (s, 1H), 6.97-6.94 (m, 1H), 6.98-6.60 (m, |
| | 1H), 6.39 (d, J = 3.6 Hz, 1H), 4.54-4.42 (m, 1H), 3.27-3.20 (m, 1H), 2.86- |
| | 2.75 (m, 1H), 2.42 (s, 3H), 2.41-2.30 (m, 2H), 2.20 (s, 3H), 2.16-2.06 |
| | (m, 1H), 1.96-1.86 (m, 1H), 1.85-1.72 (m, 1H), 1.72-1.58 (m, 1H). NH |
| | not observed. |
| 50 | MS m/z 454.3 [M + H]+; 1H NMR (400 MHz, DMSO-d₆) δ 7.89-7.79 (m, |
| | 1H), 7.54 (d, J = 8.6 Hz, 1H), 7.31 (s, 1H), 7.11 (dd, J = 8.6, 2.4 Hz, 1H), |
| | 7.05-6.99 (m, 1H), 6.91 (d, J = 3.3 Hz, 1H), 4.39-4.16 (m, 1H), 3.87 (s, |
| | 3H), 3.26-3.06 (m, 1H), 2.94-2.73 (m, 1H), 2.46-2.29 (m, 2H), 2.11- |
| | 1.70 (m, 4H), 1.67-1.36 (m, 2H), 1.12-0.92 (m, 3H). |
| 51 | MS m/z 470.2 [M + H]+; 1H NMR (400 MHz, DMSO-d₆) δ 7.83 (t, J = 3.7 |
| | Hz, 1H), 7.54 (d, J = 8.6 Hz, 1H), 7.32-7.22 (m, 1H), 7.11 (dd, J = 8.6, 2.4 |
| | Hz, 1H), 7.05-6.99 (m, 1H), 6.91 (d, J = 3.3 Hz, 1H), 4.55-4.34 (m, 1H), |
| | 4.33-4.19 (m, 1H), 3.87 (s, 3H), 3.58-3.45 (m, 2H), 3.25-3.07 (m, 1H), |
| | 2.94-2.72 (m, 1H), 2.48-2.37 (m, 2H), 2.15-1.92 (m, 3H), 1.81-1.68 |
| | (m, 1H), 1.66-1.50 (m, 1H), 1.44 (t, J = 10.2 Hz, 1H). |
| 52 | MS m/z 458.2 [M + H]+; 1H NMR (400 MHz, Methanol-d4) δ 7.65 (t, J = |
| | 3.7 Hz, 1H), 7.52 (d, J = 8.5 Hz, 1H), 7.08 (dd, J = 8.5, 2.3 Hz, 1H), 7.00- |
| | 6.94 (m, 1H), 6.79 (d, J = 3.3 Hz, 1H), 5.10-4.92 (m, 1H), 4.82-4.69 (m, |
| | 1H), 3.90 (s, 3H), 3.30-3.27 (m, 1H), 3.12-3.02 (m, 1H), 2.54-2.36 (m, |
| | 5H), 2.34-2.22 (m, 1H), 2.01-1.76 (m, 1H). NH not observed. |
| 53 | MS m/z 472.2 [M + H]+; 1H NMR (400 MHz, Methanol-d4) δ 7.65 (t, J = |
| | 3.6 Hz, 1H), 7.53 (d, J = 7.9 Hz, 1H), 7.12-7.05 (m, 1H), 6.99-6.94 (m, |
| | 1H), 6.79 (d, J = 3.3 Hz, 1H), 5.14-4.92 (m, 1H), 4.81-4.70 (m, 1H), 3.90 |
| | (s, 3H), 3.41-3.33 (m, 1H), 3.22-3.07 (m, 1H), 2.63 (q, 2H), 2.56-2.40 |
| | (m, 2H), 2.36-2.23 (m, 1H), 2.02-1.77 (m, 1H), 1.16 (t, J = 7.2 Hz, 3H). |
| | NH not observed. |
| 54 | MS m/z 427.2 [M + H]+. 1H NMR (400 MHz, Methanol-d4) δ 7.68 (t, J = 3.7 |
| | Hz, 1H), 7.52 (d, J = 8.6 Hz, 1H), 7.07 (dd, J = 8.6, 2.4 Hz, 1H), 7.00-6.93 |
| | (m, 1H), 6.78 (d, J = 3.4 Hz, 1H), 4.23 (quint, J = 8.0 Hz, 1H), 3.90 (s, 3H), |
| | 2.69-2.59 (m, 2H), 2.30-2.19 (m, 2H), 1.43 (s, 3H). 2H not observed |
| | (NH and OH). |
| 55 | MS m/z 422.3 [M + H]+; 1H NMR (400 MHz, Methanol-d₄) δ 7.67 (t, J = 3.7 |
| | Hz, 1H), 7.45 (d, J = 8.5 Hz, 1H), 6.94 (dd, J = 8.5, 2.3 Hz, 1H), 6.83 (d, J = |
| | 2.3 Hz, 1H), 6.80 (t, J = 73.7 Hz, 1H) 6.78 (d, J = 3.3 Hz, 1H), 4.56-4.39 |
| | (m, 1H), 3.89 (s, 3H), 3.70-3.52 (m, 1H), 3.25-3.14 (m, 1H), 2.98-2.68 |
| | (m, 5H), 2.28-2.02 (m, 2H), 1.97-1.66 (m, 2H). NH not observed. |
| 56 | MS m/z 436.3 [M + H]+; 1H NMR (400 MHz, Methanol-d₄) δ 7.67 (t, J = 3.7 |
| | Hz, 1H), 7.45 (d, J = 8.6 Hz, 1H), 6.94 (dd, J = 8.5, 2.3 Hz, 1H), 6.83 (d, J = |
| | 2.1 Hz, 1H), 6.80 (t, J = 74.1 Hz, 1H), 6.78 (d, J = 3.3 Hz, 1H), 4.55-4.40 |
| | (m, 1H), 3.89 (s, 3H), 3.68-3.53 (m, 1H), 3.26-3.14 (m, 1H), 3.01-2.86 |
| | (m, 2H), 2.79-2.58 (m, 2H), 2.27-2.13 (m, 1H), 2.10-1.98 (m, 1H), 1.95- |
| | 1.69 (m, 2H), 1.28 (t, J = 7.3 Hz, 3H). NH not observed. |
| 57 | MS m/z 452.3 [M + H]+; 1H NMR (400 MHz, Methanol-d₄) δ 7.67 (t, J = 3.7 |
| | Hz, 1H), 7.45 (d, J = 8.5 Hz, 1H), 6.95 (dd, J = 8.5, 2.3 Hz, 1H), 6.83 (d, J = |
| | 2.3 Hz, 1H), 6.80 (t, J = 73.7 Hz, 1H), 6.79 (d, J = 3.4 Hz, 1H), 4.60-4.50 |
| | (m, 1H), 3.92-3.84 (m, 5H), 3.83-3.72 (m, 1H), 3.45-3.35 (m, 1H), 3.21- |
| | 3.09 (m, 2H), 3.03-2.82 (m, 2H), 2.27-2.06 (m, 2H), 2.02-1.75 (m, |
| | 2H). 2H not observed (NH and OH). |
| 58 | MS m/z 409.2 [M + H]+; 1H NMR (400 MHz, Methanol-d₄) δ 7.67 (t, J = 3.7 |
| | Hz, 1H), 7.45 (d, J = 8.5 Hz, 1H), 6.94 (dd, J = 8.6, 2.4 Hz, 1H), 6.82 (d, J = |
| | 2.3 Hz, 1H), 6.78 (t, J = 73.7 Hz, 1H), 6.76 (d, J = 3.4 Hz, 1H), 4.22 (quint., |
| | J = 8.1 Hz, 1H), 3.89 (s, 3H), 2.72-2.57 (m, 2H), 2.24 (t, J = 10.3 Hz, 2H), |
| | 1.43 (s, 3H). |
| 59 | MS m/z 436.3 [M + H]+; 1H NMR (500 MHz, Methanol-d₄) δ 7.21 (dd, J = |
| | 3.0, 1.2 Hz, 1H), 6.95 (d, J = 8.6 Hz, 1H), 6.50 (dd, J = 8.6, 2.5 Hz, 1H), |
| | 6.41 (dq, J = 2.9, 1.4 Hz, 1H), 6.36 (dd, J = 3.9, 2.9 Hz, 1H), 5.94 (dd, J = |
| | 4.0, 1.2 Hz, 1H), 3.88 (tt, J = 8.7, 3.8 Hz, 1H), 2.74 (s, 3H), 2.62 (s, 1H), |
| | 2.26-2.12 (m, 1H), 1.99-1.86 (m, 2H), 1.76-1.60 (m, 2H), 1.60-1.46 |
| | (m, 1H), 1.26 (dq, J = 13.1, 4.5 Hz, 1H), 1.16 (dtt, J = 13.9, 10.2, 3.8 Hz, |
| | 1H), 1.04 (d, J = 11.1 Hz, 1H), 0.55 (t, J = 7.2 Hz, 3H). NH not observed. |
| 60 | MS m/z 440.3 [M + H]+; 1H NMR (500 MHz, Methanol-d₄) δ 7.78 (dd, J = |
| | 2.9, 1.2 Hz, 1H), 7.55 (d, J = 8.7 Hz, 1H), 7.10 (dd, J = 8.6, 2.5 Hz, 1H), |
| | 7.01 (dq, J = 2.8, 1.5 Hz, 1H), 6.96 (t, J = 3.4 Hz, 1H), 6.55 (dd, J = 4.0, 1.2 |
| | Hz, 1H), 5.09-4.89 (m, 1H), 4.79 (dp, J = 14.6, 4.2 Hz, 1H), 3.91 (s, 3H), |
| | 3.29-3.17 (m, 1H), 3.08-2.92 (m, 1H), 2.48-2.38 (m, 1H), 2.36 (s, 3H), |
| | 2.24 (t, J = 10.2 Hz, 1H), 1.92 (t, J = 11.8 Hz, 1H), 1.84 (t, J = 13.0 Hz, 1H). |
| | NH not observed. |
| 61 | MS m/z 452.3 [M + H]+; 1H NMR (500 MHz, Methanol-d₄) δ 7.75 (dd, J = |
| | 2.9, 1.2 Hz, 1H), 7.46 (d, J = 8.5 Hz, 1H), 7.00 (dd, J = 8.6, 2.5 Hz, 1H), |
| | 6.91 (dq, J = 3.0, 1.5 Hz, 1H), 6.88-6.74 (m, 1H), 6.44 (dd, J = 4.0, 1.2 Hz, |
| | 1H), 4.45-4.28 (m, 1H), 3.81 (s, 3H), 3.71-3.54 (m, 2H), 3.07 (m, 1H), |
| | 2.72-2.60 (m, 1H), 2.51 (dtd, J = 24.3, 12.6, 5.8 Hz, 2H), 2.33 (s, 2H), |
| | 2.01-1.87 (m, 1H), 1.77 (t, J = 6.9 Hz, 1H), 1.71-1.52 (m, 2H). NH and |
| | OH not observed |
| 62 | MS m/z 423.3 [M + H]+; 1H NMR (500 MHz, Methanol-d₄) δ 7.79 (dd, J = |
| | 2.9, 1.2 Hz, 1H), 7.56 (d, J = 8.6 Hz, 1H), 7.09 (dd, J = 8.5, 2.5 Hz, 1H), |
| | 7.05-6.97 (m, 1H), 6.95 (t, J = 3.5 Hz, 1H), 6.53 (dd, J = 3.9, 1.2 Hz, 1H), |
| | 4.24 (tt, J = 11.3, 3.9 Hz, 1H), 3.91 (s, 3H), 3.74 (tt, J = 10.7, 4.1 Hz, 1H), |
| | 2.43 (d, J = 12.0 Hz, 1H), 2.18-2.07 (m, 1H), 1.98 (d, J = 12.4 Hz, 1H), |
| | 1.88 (dt, J = 13.1, 3.6 Hz, 1H), 1.55-1.39 (m, 2H), 1.27 (dt, J = 16.4, 10.4 |
| | Hz, 2H). NH and OH peak not observed. |
| 63 | MS m/z 403.2 [M + H]+; 1H NMR (400 MHz, Methanol-d₄) δ 7.80 (s, 2H), |
| | 7.76-7.70 (m, 1H), 7.66 (s, 1H), 7.01-6.97 (m, 1H), 6.90 (t, J = 73.17 Hz, |
| | 1H), 6.57 (dd, J = 3.9, 1.0 Hz, 1H), 4.25-4.15 (m, 1H), 3.74 (dd, J = 13.8, |
| | 4.1 Hz, 1H), 3.56 (dd, J = 13.6, 7.6 Hz, 1H), 1.28 (d, J = 6.3 Hz, 3H), 2H |
| | not observed (NH and OH). |
| 64 | MS m/z 369.0 [M + H]+; 1H NMR (400 MHz, Methanol-d₄) δ 7.75 (dd, J = |
| | 2.8 Hz, 0.8 Hz, 1H), 7.56 (d, J = 8.8 Hz, 1H), 7.10 (dd, J = 8.4, 3.6 Hz, 1H), |
| | 7.01 (br s, 1H), 6.96 (t, J = 4.0 Hz, 1H), 6.54 (dd, J = 4.0, 0.8 Hz, 1H), 3.91 |
| | (s, 3H), 3.89 (t, J = 5.6 Hz, 2H), 3.79 (t, J = 5.6 Hz, 2H). NH and OH not |
| | observed. |
| 65 | MS m/z 383.1 [M + H]+; 1H NMR (400 MHz, Methanol-d₄) δ 7.77 (dd, J = |
| | 2.8, 0.8 Hz, 1H), 7.55 (d, J = 8.8 Hz, 1H), 7.10 (dd, J = 8.4, 2.4 Hz, 1H), |
| | 7.04-7.00 (m, 1H), 6.97 (dd, J = 4.0, 2.8 Hz, 1H), 6.55 (dd, J = 4.0, 1.2 Hz, |
| | 1H), 4.24-4.14 (m, 1H), 3.91 (s, 3H), 3.73 (dd, J = 14.0, 4.0 Hz, 1H), 3.54 |
| | (dd, J = 13.6, 7.6 Hz, 1H), 1.27 (d, J = 6.4 Hz, 3H). NH and OH not |
| | observed. |
| 66 | MS m/z 397.1 [M + H]+; 1H NMR (400 MHz, Methanol-d₄) δ 7.80 (dd, J = |
| | 2.8, 1.2 Hz, 1H), 7.55 (d, J = 8.8 Hz, 1H), 7.09 (dd, J = 8.4, 2.4 Hz, 1H), |
| | 7.01 (br s, 1H), 6.98 (t, J = 7.2 Hz, 1H), 6.56 (d, J = 3.2 Hz, 1H), 3.90 (s, |
| | 3H), 3.71 (s, 2H), 1.31 (s, 6H). NH and OH not observed. |
| 67 | MS m/z 409.1 [M + H]+; 1H NMR (400 MHz, Methanol-d₄) δ 7.82 (dd, J = |
| | 1.2 Hz, 3.2 Hz, 1H), 7.55 (d, J = 8.4 Hz, 1H), 7.07-7.10 (m, 1H), 7.01 |
| | (s, 1H), 6.94 (t, J = 1.2 Hz, 1H), 6.53 (dd, J = 1.2 Hz, 4.0 Hz, 1H), 4.42- |
| | 4.37(m, 1H), 4.02-4.05 (m, 2H), 3.90 (s, 3H), 3.62-3.56 (m, 2H), 2.13- |
| | 2.16 (m, 2H), 1.72-1.82 (m, 2H). NH not observed. |
| 68 | MS m/z 395.2 [M + H]+; 1H NMR (400 MHz, Methanol-d₄) δ 7.85 (dd, J = |
| | 2.8, 1.2 Hz, 1H), 7.56 (d, J = 8.4 Hz, 1H), 7.09 (dd, J = 8.4, 2.4 Hz, 1H), |
| | 7.04-6.99 (m, 1H), 6.95 (t, J = 3.6 Hz, 1H), 6.55 (dd, J = 4.0, 0.8 Hz, 1H), |
| | 4.85-4.77 (m, 1H), 4.14-4.02 (m, 2H), 3.95-3.85 (m, 5H), 2.50-2.36 |
| | (m, 1H), 2.22-2.10 (m, 1H). NH not observed. |
| 69 | MS m/z 419.1 [M + H]+; 1H NMR (400 MHz, Methanol-d₄) δ 7.78 (dd, J = |
| | 2.8, 1.2 Hz, 1H), 7.55 (d, J = 8.4 Hz, 1H), 7.10 (dd, J = 8.4, 2.4 Hz, 1H), |
| | 7.07 (d, J = 1.2 Hz, 1H), 7.04-7.00 (m, 1H), 6.96 (t, J = 3.2 Hz, 1H), 6.91 |
| | (d, J = 1.2 Hz, 1H), 6.56 (dd, J = 4.0, 0.8 Hz, 1H), 4.91 (s, 2H), 3.91 (s, 3H), |
| | 3.84 (s, 3H). NH not observed. |
| 70 | MS m/z 383.1 [M + H]+; 1H NMR (400 MHz, Methanol-d₄) δ 7.77 (dd, J = |
| | 2.8, 0.8 Hz, 1H), 7.56 (d, J = 8.4 Hz, 1H), 7.10 (dd, J = 8.4, 2.4 Hz, 1H), |
| | 7.01 (br s, 1H), 6.96 (t, J = 3.6 Hz, 1H), 6.55 (dd, J = 4.0, 0.8 Hz, 1H), 4.24- |
| | 4.14 (m, 1H), 3.91 (s, 3H), 3.73 (dd, J = 14.0, 4.0 Hz, 1H), 3.54 (dd, J = |
| | 13.6, 7.6 Hz, 1H), 1.27 (d, J = 6.0 Hz, 3H). NH and OH not observed. |
| 71 | MS m/z 393.2 [M + H]+; 1H NMR (400 MHz, Methanol-d₄) δ 7.82 (d, J = |
| | 1.9 Hz, 1H), 7.48 (d, J = 8.5 Hz, 1H), 7.36 (d, J = 2.4 Hz, 1H), 7.30 (dd, J = |
| | 8.6, 2.4 Hz, 1H), 6.93 (t, J = 3.4 Hz, 1H), 6.38 (d, J = 3.8 Hz, 1H), 4.31- |
| | 4.21 (m, 1H), 3.94 (s, 3H), 2.69-2.62 (m, 2H), 2.30-2.22 (m, 2H), 1.44 |
| | (s, 3H). 2 H not observed (NH and OH) |
| 72 | MS m/z 359.2, 361.1 [M + H]+; 1H NMR (400 MHz, Methanol-d₄) δ 7.84 (d, |
| | J = 2.0 Hz, 1H), 7.43 (d, J = 8.5 Hz, 1H), 7.15 (d, J = 2.5 Hz, 1H), 7.04 (dd, |
| | J = 8.5, 2.4 Hz, 1H), 6.96 (t, J = 3.4 Hz, 1H), 6.50 (d, J = 3.9 Hz, 1H), 4.31- |
| | 4.21 (m, 1H), 3.89 (s, 3H), 2.69-2.69 (m, 2H), 2.31-2.23 (m, 2H), 1.44 |
| | (s, 3H). 2 H not observed (NH and OH) |
| 73 | MS m/z 409.1 [M + H]+; 1H NMR (400 MHz, Methanol-d₄) δ 7.67 (dd, J = |
| | 2.8, 1.2 Hz, 1H), 7.56 (d, J = 8.8 Hz, 1H), 7.10 (dd, J = 8.4, 2.4 Hz, 1H), |
| | 7.04-6.99 (m, 1H), 6.95 (t, J = 3.2 Hz, 1H), 6.54 (dd, J = 4.0, 0.8 Hz, 1H), |
| | 4.39-4.31 (m, 1H), 3.97-3.92 (m, 1H), 3.91 (s, 3H), 3.85-3.76 (m, 2H), |
| | 3.64 (dd, J = 14.0, 8.0 Hz, 1H), 2.16-2.07 (m, 1H), 2.06-1.90 (m, 2H), |
| | 1.78-1.68 (m, 1H). NH not observed. |
| 74 | MS m/z 422.1 [M + H]+; 1H NMR (400 MHz, Methanol-d₄) δ 8.22 (br s, |
| | 1H), 7.76 (d, J = 8.4 Hz, 1H), 7.39-7.34 (m, 1H), 7.34-7.29 (m, 1H), 7.25 |
| | (dd, J = 8.8, 2.4 Hz, 1H), 7.19 (br s, 1H), 4.18 (dd, J = 15.2, 4.4 Hz, 1H), |
| | 4.03 (dd, J = 15.2, 4.4 Hz, 1H), 3.96 (s, 3H), 3.86-3.72 (m, 2H), 3.28- |
| | 3.19 (m, 1H), 3.12 (s, 3H), 2.48-2.36 (m, 1H), 2.25-2.01 (m, 3H). NH |
| | not observed. |
| 75 | MS m/z 396.2 [M + H]+; 1H NMR (400 MHz, Methanol-d₄) δ 7.81 (d, J = |
| | 2.4 Hz, 1H), 6.93 (t, J = 3.2 Hz, 1H), 6.65 (dd, J = 11.6, 2.4 Hz, 1H), 6.43 |
| | (d, J = 4.0 Hz, 1H), 6.41 (d, J = 2.0 Hz, 1H), 4.54-4.42 (m, 1H), 3.83 (s, |
| | 3H), 3.20-3.06 (m, 1H), 2.76-2.64 (m, 1H), 2.34 (s, 3H), 2.31-2.19 (m, |
| | 2H), 2.15-2.00 (m, 1H), 1.91-1.82 (m, 1H), 1.82-1.72 (m, 1H), 1.72- |
| | 1.55 (m, 2H), 0.85-0.69 (m, 2H), 0.69-0.60 (m, 1H), 0.59-0.50 (m, 1H). |
| | NH not observed. |
| 76 | MS m/z 409.0 [M + H]+; 1H NMR (400 MHz, Methanol-d₄) δ 7.76 (dd, J = |
| | 3.2, 1.2 Hz, 1H), 7.55 (d, J = 8.8 Hz, 1H), 7.09 (dd, J = 8.8, 2.4 Hz, 1H), |
| | 7.01 (d, J = 1.2 Hz, 1H), 6.95 (t, J = 3.2 Hz, 1H), 6.53 (dd, J = 4.0, 1.2 Hz, |
| | 1H), 4.39-4.31 (m, 1H), 3.98-3.92 (m, 1H), 3.90 (s, 3H), 3.85-3.77 (m, |
| | 2H), 3.63 (dd, J = 14.0, 8.0 Hz, 1H), 2.16-2.06 (m, 1H), 2.05-1.88 (m, |
| | 2H), 1.78-1.68 (m, 1H). NH not observed. |
| 77 | MS m/z 444.3 [M + H]+; 1H NMR (400 MHz, Methanol-d₄) δ 7.90 (s, 1H), |
| | 7.86-7.81 (m, 2H), 7.80-7.73 (m, 1H), 7.01 (t, J = 3.4 Hz, 1H), 6.56 (d, |
| | J = 3.8 Hz, 1H), 5.06 (t, J = 9.5 Hz, 1H), 3.83 (q, J = 10.9 Hz, 2H), 3.56- |
| | 3.43 (m, 2H), 2.80-2.70 (m, 1H), 2.35 (dd, J = 12.3, 9.9 Hz, 1H). 2H not |
| | observed (2NH) |
| 78 | MS m/z 443.3 [M + H]+; 1H NMR (400 MHz, Methanol-d₄) δ 7.91-7.87 (m, |
| | 1H), 7.80 (d, J = 8.0 Hz, 1H), 7.77-7.71 (m, 1H), 7.66 (s, 1H), 7.11-6.71 |
| | (m, 2H), 6.56 (d, J = 3.9 Hz, 1H), 4.24-4.11 (m, 1H), 3.68 (dt, J = 4.3, 10.0 |
| | Hz, 1H), 2.34-2.21 (m, 1H), 2.19-2.07 (m, 1H), 1.91-1.74 (m, 2H), 1.56- |
| | 1.35 (m, 4H); 2H not observed (NH and OH). |
| 79 | MS m/z 443.3 [M + H]+; 1H NMR (400 MHz, Methanol-d₄) δ 7.97-7.93 (m, |
| | 1H), 7.80 (d, J = 8.0 Hz, 1H), 7.77-7.72 (m, 1H), 7.67 (s, 1H), 7.13-6.71 |
| | (m, 2H), 6.56 (d, J = 3.9 Hz, 1H), 4.35-4.25 (m, 2H), 2.04-1.90 (m, 2H), |
| | 1.90-1.81 (m, 2H), 1.80-1.64 (m, 2H), 1.60-1.44 (m, 2H); 2H not |
| | observed (NH and OH). |
| 80 | MS m/z 389.2 [M + H]+; 1H NMR (400 MHz, Methanol-d₄) δ 7.85 (d, J = |
| | 1.9 Hz, 1H), 7.44 (d, J = 7.8 Hz, 1H), 7.26-7.16 (m, 2H), 6.97-6.55 (m, |
| | 2H), 6.55-6.48 (m, 1H), 4.21-4.08 (m, 1H), 3.66 (dt, J = 4.3, 10.0 Hz, |
| | 1H), 2.47 (s, 3H), 2.30-2.21 (m, 1H), 2.21-2.04 (m, 1H), 1.89-1.74 (m, |
| | 2H), 1.57-1.35 (m, 4H); 2H not observed (NH and OH). |
| 81 | MS m/z 423.3 [M + H]+; 1H NMR (400 MHz, Methanol-d₄) δ 7.86 (d, J = |
| | 2.0 Hz, 1H), 7.57 (d, J = 8.5 Hz, 1H), 7.11 (dd, J = 2.4, 8.6 Hz, 1H), 7.03 (s, |
| | 1H), 7.00-6.92 (m, 1H), 6.54 (d, J = 3.8 Hz, 1H), 4.89 (s, 3H), 4.23-4.08 |
| | (m, 1H), 3.66 (dt, J = 4.3, 10.0 Hz, 1H), 2.34-2.04 (m, 2H), 1.89-1.72 (m, |
| | 2H), 1.59-1.36 (m, 4H); 2H not observed (NH and OH). |
| 82 | MS m/z 404.3, 406.2 [M + H]+; 1H NMR (400 MHz, Methanol-d4) δ 7.68 |
| | (t, J = 3.7 Hz, 1H), 7.40 (d, J = 8.5 Hz, 1H), 7.12 (d, J = 2.4 Hz, 1H), 7.02 |
| | (dd, J = 8.4, 2.4 Hz, 1H), 6.78 (d, J = 3.2 Hz, 1H), 4.55-4.40 (m, 1H), 3.88 |
| | (s, 3H), 3.60-3.42 (m, 1H), 3.20-3.03 (m, 1H), 2.92-2.75 (m, 2H), 2.68- |
| | 2.43 (m, 2H), 2.26-2.10 (m, 1H), 2.05-1.93 (m, 1H), 1.89-1.64 (m, |
| | 2H), 1.24 (t, J = 7.2 Hz, 3H). NH not observed. |
| 83 | MS m/z 420.2, 422.2 [M + H]+; 1H NMR (400 MHz, Methanol-d₄) δ 7.70 (t, |
| | J = 3.7 Hz, 1H), 7.40 (d, J = 8.5 Hz, 1H), 7.12 (d, J = 2.4 Hz, 1H), 7.02 (dd, |
| | J = 8.5, 2.4 Hz, 1H), 6.78 (d, J = 3.3 Hz, 1H), 4.57-4.46 (m, 1H), 3.88 (s, |
| | 3H), 3.81 (t, J = 5.2 Hz, 2H), 3.62-3.50 (m, 1H), 3.22-3.08 (m, 1H), 3.03- |
| | 2.88 (m, 2H), 2.82-2.63 (m, 2H), 2.22-2.08 (m, 1H), 2.06-1.95 (m, |
| | 1H), 1.93-1.71 (m, 2H). 2H not observed (NH and OH). |
| 84 | MS m/z 377.2, 379.1 [M + H]+; 1H NMR (400 MHz, Methanol-d₄) δ 7.67 (t, |
| | J = 3.7 Hz, 1H), 7.39 (d, J = 8.5 Hz, 1H), 7.11 (d, J = 2.4 Hz, 1H), 7.01 (dd, |
| | J = 8.5, 2.4 Hz, 1H), 6.76 (d, J = 3.2 Hz, 1H), 4.34-4.14 (m, 1H), 3.88 (s, |
| | 3H), 2.69-2.56 (m, 2H), 2.30-2.17 (m, 2H), 1.43 (s, 3H). 2H not |
| | observed (NH and OH). |
| 85 | MS m/z 472.3 [M + H]+; 1H NMR (500 MHz, Methanol-d₄) δ 7.89-7.79 |
| | (m, 1H), 7.69 (d, J = 8.5 Hz, 1H), 7.33 (dd, J = 8.6, 2.3 Hz, 1H), 7.29 (s, |
| | 1H), 7.02 (t, J = 75 Hz, 1H), 6.97 (t, J = 3.5 Hz, 1H), 6.55 (d, J = 4.0 Hz, |
| | 1H), 4.56-4.44 (m, 1H), 3.36 (tp, J = 4.0, 1.9 Hz, 2H), 2.84-2.74 (m, 1H), |
| | 2.53 (s, 1H), 2.33-2.22 (m, 1H), 2.17-2.04 (m, 1H), 1.94-1.80 (m, 1H), |
| | 1.83-1.71 (m, 1H), 1.69-1.57 (m, 1H), 1.36-1.24 (m, 1H), 1.15 (t, J = |
| | 7.3 Hz, 3H). NH peak not observed. |
| 86 | MS m/z 488.3 [M + H]+; 1H NMR (500 MHz, Methanol-d₄) δ 7.87 (dd, J = |
| | 2.9, 1.2 Hz, 1H), 7.69 (d, J = 8.5 Hz, 1H), 7.33 (dd, J = 8.5, 2.4 Hz, 1H), |
| | 7.29 (s, 1H), 7.02 (t, J = 75 Hz, 1H), 6.97 (t, J = 3.4 Hz, 1H), 6.55 (dd, J = |
| | 3.9, 1.2 Hz, 1H), 4.47 (dd, J = 8.8, 4.5 Hz, 1H), 3.78-3.65 (m, 2H), 3.19- |
| | 3.09 (m, 1H), 2.78-2.69 (m, 1H), 2.63 (dt, J = 12.3, 6.0 Hz, 1H), 2.56 (dt, |
| | J = 12.5, 5.7 Hz, 1H), 2.48-2.32 (m, 2H), 2.10-1.98 (m, 1H), 1.86 (dd, J = |
| | 14.1, 5.2 Hz, 1H), 1.80-1.62 (m, 2H). NH and OH peak not observed. |
| 87 | MS m/z 476.2 [M + H]+; 1H NMR (500 MHz, Methanol-d₄) δ 7.81 (dd, J = |
| | 2.9, 1.2 Hz, 1H), 7.69 (d, J = 8.5 Hz, 1H), 7.33 (dd, J = 8.5, 2.4 Hz, 1H), |
| | 7.29 (s, 1H), 7.02 (t, J = 75 Hz, 1H), 6.98 (t, J = 3.5 Hz, 1H), 6.56 (d, J = 3.9 |
| | Hz, 1H), 5.08-4.90 (m, 1H), 4.80 (ddd, J = 12.7, 10.4, 4.1 Hz, 1H), 3.28- |
| | 3.19 (m, 1H), 3.01 (t, J = 11.8 Hz, 1H), 2.41 (dd, J = 12.3, 6.3 Hz, 1H), 2.36 |
| | (s, 3H), 2.24 (t, J = 10.3 Hz, 1H), 1.92 (t, J = 12.4 Hz, 1H), 1.84 (t, J = 12.2 |
| | Hz, 1H). NH peak not observed. |
| 88 | MS m/z 445.2 [M + H]+.. 1H NMR (500 MHz, Methanol-d₄) δ 7.84 (dd, J = |
| | 3.0, 1.4 Hz, 1H), 7.69 (d, J = 8.5 Hz, 1H), 7.34 (d, J = 8.6 Hz, 1H), 7.30 (s, |
| | 1H), 7.03 (t, J = 75 Hz, 1H) 6.97 (m, 1H), 6.55 (d, J = 3.8 Hz, 1H), 4.28 (p, |
| | J = 7.9 Hz, 1H), 2.74-2.52 (m, 2H), 2.37-2.17 (m, 2H), 1.44 (s, 3H). NH |
| | and OH peak not observed. |
| 89 | MS m/z 465.2 [M + H]+; 1H NMR (400 MHz, Methanol-d₄) δ 7.99 (dd, J = |
| | 0.9, 2.8 Hz, 1H), 7.78 (d, J = 8.0 Hz, 1H), 7.74-7.69 (m, 1H), 7.65 (s, 1H), |
| | 7.21-7.11 (m, 1H), 7.09-6.65 (m, 5H), 6.57 (d, J = 3.9 Hz, 1H), 5.52 (q, |
| | J = 7.0 Hz, 1H), 1.71 (d, J = 7.0 Hz, 3H); NH and OH peak not observed. |
| 90 | MS m/z 459.2 [M + H]+; 1H NMR (500 MHz, Methanol-d₄) δ 7.92-7.88 |
| | (m, 2H), 7.83 (d, J = 8.2 Hz, 1H), 7.80-7.74 (m, 1H), 6.99 (t, J = 3.4 Hz, |
| | 1H), 6.53 (d, J = 3.5 Hz, 1H), 4.21-4.11 (m, 1H), 3.92-3.77 (m, 2H), 3.66 |
| | (td, J = 10.1, 4.3 Hz, 1H), 2.30-2.20 (m, 1H), 2.20-2.03 (m, 1H), 1.87- |
| | 1.73 (m, 2H), 1.52-1.36 (m, 4H). 2H not observed (NH and OH). |
| 91 | MS m/z 406.0 [M + H]+; 1H NMR (400 MHz, Methanol-d₄) δ 7.90 (s, 1H), |
| | 7.78 (dd, J = 2.8, 1.2 Hz, 1H), 7.55 (d, J = 8.8 Hz, 1H), 7.14 (s, 1H), 7.10 |
| | (dd, J = 8.4, 2.4 Hz, 1H), 7.04-6.97 (m, 2H), 6.59 (dd, J = 4.0, 0.8 Hz, 1H), |
| | 4.99 (s, 2H), 3.91 (s, 3H). NH not observed. |
| 92 | MS m/z 420.0 [M + H]+; 1H NMR (400 MHz, Methanol-d₄) δ 8.45 (s, 1H), |
| | 7.78 (dd, J = 2.8, 1.2 Hz, 1H), 7.55 (d, J = 8.4 Hz, 1H), 7.10 (dd, J = 8.4, 2.4 |
| | Hz, 1H), 7.04-6.97 (m, 2H), 6.58 (d, J = 4.0 Hz, 1H), 5.01 (s, 2H), 3.91 (s, |
| | 3H), 3.91 (s, 3H). NH not observed. |
| 93 | MS m/z 420.0 [M + H]+; 1H NMR (400 MHz, Methanol-d₄) δ 7.85 (s, 1H), |
| | 7.77 (dd, J = 2.8, 0.8 Hz, 1H), 7.54 (d, J = 8.8 Hz, 1H), 7.09 (dd, J = 8.4, 2.4 |
| | Hz, 1H), 7.03-6.97 (m, 2H), 6.58 (dd, J = 4.4, 0.4 Hz, 1H), 4.96 (s, 2H), |
| | 4.09 (s, 3H), 3.91 (s, 3H). NH not observed. |
| 94 | MS m/z 421.1 [M + H]+; 1H NMR (400 MHz, Methanol-d₄) δ 7.73 (dd, J = |
| | 2.8, 1.2 Hz, 1H), 7.56 (d, J = 8.4 Hz, 1H), 7.10 (dd, J = 8.8, 2.4 Hz, 1H), |
| | 7.01 (s, 1H), 6.94 (t, J = 3.6 Hz, 1H), 6.53 (dd, J = 4.0, 0.8 Hz, 1H), 3.91 (s, |
| | 3H), 3.70 (s, 2H), 2.23 (s, 6H). NH and OH not observed. |
| 95 | MS m/z 430.1 [M + H]+; 1HNMR (400 MHz, Methanol-d₄) δ 8.46 (br s, |
| | 1.2H), 8.02 (d, J = 8.0 Hz, 1H), 7.92 (s, 1H), 7.87 (d, J = 2.0 Hz, 1H), 7.71 |
| | (d, J = 8.0 Hz, 1H), 6.99 (t, J = 3.2 Hz, 1H), 6.88 (t, J = 73.6 Hz, 1H), 6.58 |
| | (d, J = 3.6 Hz, 1H), 4.64-4.53 (m, 1H), 3.79-3.64 (d, J = 9.2 Hz, 1H), |
| | 3.38-3.32 (m, 1H), 3.13 (q J = 7.2 Hz, 2H), 3.09-2.95 (m, 2H), 2.84 (s, |
| | 3H), 2.26-2.12 (m, 2H), 2.00-1.82 (m, 2H), 1.23 (t, J = 7.2 Hz, 3H). NH |
| | not observed. |
| 96 | MS m/z 481.2 [M + H]+; 1H NMR (500 MHz, Methanol-d₄) δ 8.01 (d, J = |
| | 2.0 Hz, 1H), 7.88 (s, 1H), 7.80 (d, J = 7.9 Hz, 1H), 7.75 (d, J = 8.54 Hz, |
| | 1H), 7.15 (t, J = 7.9 Hz, 1H), 7.00 (t, J = 3.3 Hz, 1H), 6.96 (d, J = 7.8 Hz, |
| | 1H), 6.93 (s, 1H), 6.66 (d, J = 7.9 Hz, 1H), 6.54 (d, J = 3.7 Hz, 1H), 5.50 (q, |
| | J = 7.3 Hz, 1H), 3.94-3.70 (m, 2H), 1.70 (d, J = 7.2 Hz, 3H). 2H not |
| | observed (NH and OH). |
| 97 | MS m/z 427.2 [M + H]; 1H NMR (500 MHz, Methanol-d₄) δ 8.07-8.02 (m, |
| | 1H), 7.89 (s, 1H), 7.87-7.78 (m, 2H), 7.61 (s, 1H), 6.84 (s, 1H), 6.53 (d, |
| | J = 3.1 Hz, 1H), 6.21 (br s, 1H), 3.89 (q, J = 10.8 Hz, 2H). 2H not observed (2 |
| | NH). |
| 98 | MS m/z 458.2 [M + H]+; 1H NMR (500 MHz, Methanol-d₄) δ 7.81-7.68 (m, |
| | 3H), 7.66 (s, 1H), 7.01 (t, J = 3.4 Hz, 1H), 6.92 (t, J = 72.78 Hz, 1H), 6.60 |
| | (d, J = 3.5 Hz, 1H), 4.68-4.61 (m, 1H), 4.17-4.07 (m, 1H), 3.43-3.33 |
| | (m, 1H), 3.16 (d, J = 11.1 Hz, 1H), 3.06-2.84 (m, 1H), 2.71-2.62 (m, |
| | 4H), 2.38-2.28 (m, 1H), 1.94-1.81 (m, 1H). 2H not observed (NH and |
| | OH). |
| 99 | MS m/z 445.3 [M + H]+; 1H NMR (400 MHz, Methanol-d₄) δ 8.19 (s, 1H), |
| | 8.17-8.08 (m, 1H), 7.92 (d, J = 2.9 Hz, 1H), 7.89-7.81 (m, 1H), 6.98 (t, |
| | J = 3.4 Hz, 1H), 6.43 (d, J = 3.9 Hz, 1H), 4.23-4.10 (m, 1H), 3.68 (dt, J = |
| | 4.3, 10.0 Hz, 1H), 2.35 (br s, 1H), 2.09 (br s, 1H), 1.90 (br s, 2H), 1.56- |
| | 1.36 (m, 4H); 2H not observed (NH and OH). |
| 100 | MS m/z 411.3 [M + H]+; 1H NMR (400 MHz, Methanol-d₄) δ 7.95 (d, J = 2.1 |
| | Hz, 1H), 7.42 (d, J = 7.8 Hz, 1H), 7.23-7.12 (m, 3H), 6.99-6.92 (m, 3H), |
| | 6.54-6.53 (m, 1H), 6.92-6.48 (m, 2H), 5.50 (q, J = 6.8 Hz, 1H), 2.46 (s, |
| | 3H), 1.70 (d, J = 7.0 Hz, 3H); 2H not observed (NH and OH). |
| 101 | MS m/z 451.3 [M + H]+; 1H NMR (400 MHz, Methanol-d₄) δ 7.86-7.78 (m, |
| | 2H), 7.77-7.71 (m, 1H), 7.67 (s, 1H), 7.21-7.14 (m, 1H), 7.12-6.88 (m, |
| | 4H), 6.76-6.67 (m, 1H), 6.59 (d, J = 3.9 Hz, 1H), 4.83 (s, 2H); 2H not |
| | observed (NH and OH). |
| 102 | MS m/z 465.2 [M + H]+; 1H NMR (400 MHz, Methanol-d₄) δ 7.99 (dd, J = |
| | 0.9, 2.8 Hz, 1H), 7.78 (d, J = 8.0 Hz, 1H), 7.74-7.69 (m, 1H), 7.65 (s, 1H), |
| | 7.21-7.11 (m, 1H), 7.09-6.65 (m, 5H), 6.57 (d, J = 3.9 Hz, 1H), 5.52 (q, |
| | J = 7.0 Hz, 1H), 1.71 (d, J = 7.0 Hz, 3H); 2H not observed (NH and OH). |
| 103 | MS m/z 465.2 [M + H]+; 1H NMR (400 MHz, Methanol-d₄) δ 7.99 (dd, J = |
| | 0.9, 2.8 Hz, 1H), 7.78 (d, J = 8.0 Hz, 1H), 7.74-7.69 (m, 1H), 7.65 (s, 1H), |
| | 7.21-7.11 (m, 1H), 7.09-6.65 (m, 5H), 6.57 (d, J = 3.9 Hz, 1H), 5.52 (q, |
| | J = 7.0 Hz, 1H), 1.71 (d, J = 7.0 Hz, 3H); 2H not observed (NH and OH). |
| 104 | MS m/z 365.3 [M + H]+; 1H NMR (400 MHz, Methanol-d₄) δ 7.91-7.90 |
| | (m, 1H), 7.30 (d, J = 8.3 Hz, 1H), 6.99 (t, J = 3.3 Hz, 1H), 6.90-6.89 (m, |
| | 1H), 6.65-6.57 (m, 2H), 4.25 (quint, J = 7.9 Hz, 1H), 3.84 (s, 3H), 2.65 |
| | (ddd, J = 9.4, 7.2, 2.6 Hz, 2H), 2.31-2.23 (m, 2H), 1.87-1.77 (m, 1H), |
| | 1.44 (s, 3H), 0.80-0.70 (m, 2H), 0.70-0.62 (m, 2H). 2H not observed |
| | (NH and OH). |
| 105 | MS m/z 347.2 [M + H]+; 1H NMR (400 MHz, Methanol-d₄) δ 8.02-7.98 (m, |
| | 1H), 7.59 (d, J = 2.1 Hz, 1H), 7.31 (d, J = 8.5 Hz, 1H), 6.85 (dd, J = 8.4, 2.5 |
| | Hz, 1H), 6.81 (t, J = 3.2 Hz, 1H), 6.59 (d, J = 2.3 Hz, 1H), 6.48 (d, J = 3.1 |
| | Hz, 1H), 6.20-6.17 (m, 1H), 3.83 (s, 3H), 1.98-1.89 (m, 1H), 0.86-0.78 |
| | (m, 2H), 0.69-0.62 (m, 2H). 2H not observed (2 NH). |
| 106 | MS m/z 379.3 [M + H]+; 1H NMR (400 MHz, Methanol-d₄) δ 7.89-7.82 |
| | (m, 1H), 7.31 (d, J = 8.3 Hz, 1H), 7.01-6.95 (m, 1H), 6.95-6.82 (m, 1H), |
| | 6.63-6.56 (m, 2H), 4.27-4.18 (m, 1H), 3.84 (s, 3H), 3.82-3.69 (m, 1H), |
| | 2.44 (d, J = 11.9 Hz, 1H), 2.14 (d, J = 11.4 Hz, 1H), 1.99 (d, J = 11.9 Hz, |
| | 1H), 1.93-1.79 (m, 2H), 1.53-1.34 (m, 3H), 1.34-1.23 (m, 1H), 0.82- |
| | 0.72 (m, 2H), 0.70-0.62 (m, 2H). 2H not observed (NH and OH). |
| 107 | MS m/z 445.2 [M + H]+; 1H NMR (400 MHz, Methanol-d₄) δ 8.16 (s, 1H), |
| | 8.10 (d, J = 8.1 Hz, 1H), 7.81 (d, J = 8.0 Hz, 1H), 7.64 (s, 1H), 6.25 (s, 1H), |
| | 4.25 (t, J = 7.9 Hz, 1H), 2.72-2.57 (m, 2H), 2.36-2.17 (m, 5H), 1.44 (s, |
| | 3H). NH and OH peaks not observed. |
| 108 | MS m/z 423.2 [M + H]+; 1H NMR (400 MHz, Methanol-d₄) δ 7.58 (s, 1H), |
| | 7.54 (d, J = 8.5 Hz, 1H), 7.09 (dd, J = 8.6, 2.3 Hz, 1H), 7.01 (s, 1H), 6.37 |
| | (m, 1H), 4.28-4.20 (m, 1H), 3.91 (s, 3H), 2.67-2.62 (m, 2H), 2.33 (s, 3H), |
| | 2.27-2.22 (m, 2H), 1.43 (s, 3H). NH and OH peak not observed. |
| 109 | MS m/z 458.2 [M + H]+; 1H NMR (400 MHz, Methanol-d₄) δ 8.17 (s, 1H), |
| | 8.10 (d, J = 7.9 Hz, 1H), 7.81 (d, J = 8.0 Hz, 1H), 7.65 (s, 1H), 6.26 (s, 1H), |
| | 4.47-4.49 (m, 1H), 2.91-2.88 (m, 1H), 2.55-2.42 (m, 5H), 2.32 (s, 3H), |
| | 2.15-2.10 (m, 1H), 2.01-1.89 (m, 1H), 1.87-1.73 (m, 1H), 1.73-1.57 |
| | (m, 1H), 1.39-1.29 (m, 1H). NH peak not observed. |
| 110 | MS m/z 443.5 [M + H]+; 1H NMR (400 MHz, Methanol-d₄) δ 7.89 (d, J = |
| | 2.0 Hz, 1H), 7.80 (br d, J = 8.0 Hz, 1H), 7.77-7.71 (m, 1H), 7.66 (s, 1H), |
| | 7.18-6.68 (m, 2H), 6.56 (d, J = 3.6 Hz, 1H), 4.28-4.08 (m, 1H), 3.77- |
| | 3.60 (m, 1H), 2.36-2.01 (m, 2H), 1.92-1.70 (m, 2H), 1.59-1.32 (m, 4H); |
| | 2H not observed (NH and OH). |
| 111 | MS m/z 459.2 [M + H]+; 1H NMR (400 MHz, Methanol-d₄) δ 7.92-7.88 |
| | (m, 2H), 7.85-7.75 (m, 2H), 6.99 (t, J = 3.4 Hz, 1H), 6.54 (d, J = 3.9 Hz, |
| | 1H), 4.20-4.11 (m, 1H), 3.85 (br dd, J = 10.8, 5.6 Hz, 2H), 3.71-3.62 (m, |
| | 1H), 2.31-2.22 (m, 1H), 2.17-2.08 (m, 1H), 1.87-1.75 (m, 2H), 1.54- |
| | 1.36 (m, 4H). 2H not observed (NH and OH). |
| 112 | MS m/z 461.2 [M + H]+; 1H NMR (400 MHz, Methanol-d₄) δ 7.94-7.88 |
| | (m, 2H), 7.86-7.75 (m, 2H), 7.01 (t, J = 3.4 Hz, 1H), 6.56 (d, J = 3.8 Hz, |
| | 1H), 4.34 (td, J = 10.51, 3.5 Hz, 1H), 4.04 (dd, J = 11.2, 4.9 Hz, 1H), 3.98 |
| | (br dd, J = 11.3, 4.3 Hz, 1H), 3.90-3.74 (m, 3H), 3.56 (td, J = 11.1, 1.75 |
| | Hz, 1H), 3.26 (t, J = 10.5 Hz, 1H), 2.31-2.24 (m, 1H), 1.79 (qd, J = 12.38, |
| | 5.13 Hz, 1H). 2H not observed (NH and OH). |
| 113 | MS m/z 461.2 [M + H]+; 1H NMR (400 MHz, Methanol-d₄) δ 7.94-7.88 |
| | (m, 2H), 7.86-7.75 (m, 2H), 7.01 (t, J = 3.4 Hz, 1H), 6.56 (d, J = 3.8 Hz, |
| | 1H), 4.34 (td, J = 10.51, 3.5 Hz, 1H), 4.04 (dd, J = 11.2, 4.9 Hz, 1H), 3.98 |
| | (br dd, J = 11.3, 4.3 Hz, 1H), 3.90-3.74 (m, 3H), 3.56 (td, J = 11.1, 1.75 |
| | Hz, 1H), 3.26 (t, J = 10.5 Hz, 1H), 2.31-2.24 (m, 1H), 1.79 (qd, J = 12.38, |
| | 5.13 Hz, 1H). 2H not observed (NH and OH). |
| 114 | MS m/z 461.2 [M + H]+; 1H NMR (400 MHz, Methanol-d₄) δ 8.00 (d, J = |
| | 2.4 Hz, 1H), 7.90 (s, 1H), 7.86-7.74 (m, 2H), 7.00 (t, J = 3.4 Hz, 1H), 6.55 |
| | (d, J = 3.8 Hz, 1H), 4.47 (td, J = 11.88, 3.13 Hz, 1H), 4.19-4.14 (m, 1H), |
| | 4.04 (dd, J = 11.6, 4.1 Hz, 1H), 3.98 (d, J = 12.5 Hz, 1H), 3.85 (q, J = 11.0 |
| | Hz, 2H), 3.70 (d, J = 12.1 Hz, 1H), 3.62 (t, J = 11.0 Hz, 1H), 2.30 (qd, J = |
| | 12.6, 4.7 Hz, 1H), 1.88-1.81 (m, 1H). 2H not observed (NH and OH). |
| 115 | MS m/z 461.2 [M + H]+; 1H NMR (400 MHz, Methanol-d₄) δ 8.00 (d, J = |
| | 2.4 Hz, 1H), 7.90 (s, 1H), 7.86-7.74 (m, 2H), 7.00 (t, J = 3.4 Hz, 1H), 6.55 |
| | (d, J = 3.8 Hz, 1H), 4.47 (td, J = 11.88, 3.13 Hz, 1H), 4.19-4.14 (m, 1H), |
| | 4.04 (dd, J = 11.6, 4.1 Hz, 1H), 3.98 (d, J = 12.5 Hz, 1H), 3.85 (q, J = 11.0 |
| | Hz, 2H), 3.70 (d, J = 12.1 Hz, 1H), 3.62 (t, J = 11.0 Hz, 1H), 2.30 (qd, J = |
| | 12.6, 4.7 Hz, 1H), 1.88-1.81 (m, 1H). 2H not observed (NH and OH). |
| 116 | MS m/z 410.2 [M + H]+; 1H NMR (400 MHz, Methanol-d4) δ 7.63 (t, J = |
| | 3.7 Hz, 1H), 7.46 (d, J = 8.4 Hz, 1H), 7.34 (d, J = 2.3 Hz, 1H), 7.29 (dd, J = |
| | 8.3, 2.6 Hz, 1H), 6.76 (d, J = 3.1 Hz, 1H), 5.09-4.94 (m, 1H), 3.93 (s, 3H), |
| | 3.57-3.39 (m, 2H), 2.81-2.67 (m, 1H), 2.39-2.21 (m, 1H). |
| 117 | MS m/z 393.1 [M + H]+. 1H NMR (400 MHz, Methanol-d4) δ 7.86 (t, J = |
| | 4.1 Hz, 1H), 7.59 (d, J = 2.1 Hz, 1H), 7.54 (d, J = 8.4 Hz, 1H), 7.34 (d, J = |
| | 2.4 Hz, 1H), 7.29 (dd, J = 8.5, 2.3 Hz, 1H), 6.61 (d, J = 3.3 Hz, 1H), 6.18 (d, |
| | J = 2.1 Hz, 1H), 3.93 (s, 3H). NH not observed. |
| 118 | MS m/z 370.1 [M + H]+; 1H NMR (400 MHz, Methanol-d₄) δ 7.81 (d, J = |
| | 2.8 Hz, 1H), 6.94 (dd, J = 3.6, 2.8 Hz, 1H), 6.77 (s, 1H), 6.67 (dd, J = 10.8, |
| | 2.0 Hz, 1H), 6.42 (d, J = 4.0 Hz, 1H), 4.56-4.43 (m, 1H), 3.85 (s, 3H), 2.90- |
| | 2.77 (m, 1H), 2.53-2.34 (m, 5H), 2.18-2.04 (m, 4H), 1.97-1.86 (m, |
| | 2H), 1.85-1.73 (m, 1H), 1.73-1.59 (m, 1H). NH not observed. |
| 119 | MS m/z 474.1 [M + H]+; 1H NMR (400 MHz, DMSO-d₆) δ 8.00 (d, J = 2.1 |
| | Hz, 1H), 7.96 (d, J = 2.6 Hz, 2H), 7.93 (s, 1H), 7.39 (d, J = 7.7 Hz, 1H), |
| | 7.01-6.94 (m, 1H), 6.51 (d, J = 3.3 Hz, 1H), 4.33 (s, 1H), 3.17 (d, J = 7.6 |
| | Hz, 1H), 2.84 (d, J = 10.4 Hz, 1H), 2.39 (q, J = 7.1 Hz, 2H), 2.03 (s, 1H), |
| | 1.99-1.86 (m, 2H), 1.77 (d, J = 12.9 Hz, 1H), 1.59 (d, J = 12.4 Hz, 1H), |
| | 1.45 (dd, J = 17.3, 6.1 Hz, 1H), 1.02 (t, J = 7.1 Hz, 3H). |
| 120 | MS m/z 490.1 [M + H]+; 1H NMR (400 MHz, DMSO-d₆) δ 8.01 (s, 1H), |
| | 7.96 (d, J = 2.3 Hz, 2H), 7.93 (s, 1H), 7.36 (d, J = 7.9 Hz, 1H), 7.01-6.95 |
| | (m, 1H), 6.51 (d, J = 3.9 Hz, 1H), 4.42 (t, J = 5.4 Hz, 1H), 4.35 (s, 1H), 3.52 |
| | (d, J = 5.5 Hz, 2H), 3.17 (s, 1H), 2.81 (s, 1H), 2.45 (d, J = 6.2 Hz, 2H), 2.06 |
| | (dd, J = 21.3, 11.1 Hz, 3H), 1.73 (s, 1H), 1.46 (s, 1H), 1.23 (s, 1H). |
| 121 | MS m/z 461.0 [M + H]+; 1H NMR (400 MHz, DMSO-d₆) δ 8.01 (d, J = 1.9 |
| | Hz, 1H), 7.97-7.91 (m, 3H), 7.41 (d, J = 7.9 Hz, 1H), 7.00-6.94 (m, 1H), |
| | 6.49 (d, J = 4.0 Hz, 1H), 4.82 (d, J = 5.1 Hz, 1H), 4.09 (s, 1H), 3.57 (s, 1H), |
| | 2.09 (s, 1H), 1.98 (s, 1H), 1.71 (s, 2H), 1.32 (s, 4H). |
| 122 | MS m/z 461.0 [M + H]+; 1H NMR (400 MHz, DMSO-d₆) δ 7.98 (d, J = 2.0 |
| | Hz, 1H), 7.97-7.91 (m, 3H), 7.48 (d, J = 7.7 Hz, 1H), 7.02-6.92 (m, 1H), |
| | 6.50 (d, J = 3.2 Hz, 1H), 4.75 (d, J = 4.5 Hz, 1H), 4.18 (s, 1H), 3.55 (d, J = |
| | 4.2 Hz, 1H), 2.29 (d, J = 11.6 Hz, 1H), 2.03 (d, J = 10.6 Hz, 1H), 1.87 (d, |
| | J = 13.1 Hz, 1H), 1.77 (d, J = 13.3 Hz, 1H), 1.39-1.30 (m, 2H), 1.25 (d, J = |
| | 12.8 Hz, 1H), 1.18-1.08 (m, 1H). |
| 123 | MS m/z 447.0 [M + H]+; 1H NMR (400 MHz, DMSO-d₆) δ 8.00 (d, J = 1.8 |
| | Hz, 1H), 7.98-7.91 (m, 3H), 7.85 (d, J = 6.1 Hz, 1H), 7.00-6.96 (m, 1H), |
| | 6.50 (d, J = 3.0 Hz, 1H), 5.09 (s, 1H), 4.25 (dd, J = 14.3, 7.1 Hz, 1H), 2.47 |
| | (d, J = 2.7 Hz, 2H), 2.21 (dd, J = 11.3, 8.8 Hz, 2H), 1.34 (s, 3H). |
| 124 | MS m/z 446.1 [M + H]+; 1H NMR (400 MHz, Methanol-d₄) δ 7.89 (s, 2H), |
| | 7.84-7.81 (m, 1H), 7.80 (s, 1H), 7.06-6.98 (m, 1H), 6.60 (d, J = 3.9 Hz, |
| | 1H), 5.07 (t, J = 9.5 Hz, 1H), 3.55-3.45 (m, 2H), 2.79-2.69 (m, 1H), 2.40- |
| | 2.28 (m, 1H). NH not observed. |
| 125 | MS m/z 421.0 [M + H]+; 1H NMR (400 MHz, Methanol-d₄) δ 7.76 (dd, J = |
| | 2.8, 1.0 Hz, 1H), 7.53 (d, J = 8.6 Hz, 1H), 7.09 (dd, J = 8.6, 2.5 Hz, 1H), |
| | 7.03-6.98 (m, 2H), 6.59 (d, J = 3.6 Hz, 1H), 5.08 (s, 2H), 4.30 (s, 3H), |
| | 3.90 (s, 3H). NH not observed. |
| 126 | MS m/z 473.2 [M + H]+; 1H NMR (400 MHz, Methanol-d₄) δ 7.76 (s, 1H), |
| | 7.69 (d, J = 8.5 Hz, 1H), 7.58 (d, J = 8.1 Hz, 1H), 7.03-6.98 (m, 1H), 6.11- |
| | 6.06 (m, 1H), 5.77 (d, J = 2.5 Hz, 1H), 4.96 (td, J = 9.0, 4.2 Hz, 1H), 4.13- |
| | 4.05 (m, 1H), 3.75-3.61 (m, 2H), 2.13-1.90 (m, 2H), 1.89-1.60 (m, |
| | 4H), 1.57-1.30 (m, 4H). 2H not observed (NH and OH). |
| 127 | MS m/z 473.2 [M + H]+; 1H NMR (400 MHz, Methanol-d₄) δ 7.75 (s, 1H), |
| | 7.69 (d, J = 8.3 Hz, 1H), 7.58 (d, J = 8.1 Hz, 1H), 7.03-6.98 (m, 1H), 6.11- |
| | 6.07 (m, 1H), 5.78 (d, J = 2.5 Hz, 1H), 4.47-4.37 (m, 1H), 3.77-3.56 |
| | (m, 3H), 2.41-2.29 (m, 1H), 2.20-2.09 (m, 1H), 1.85-1.66 (m, 4H), 1.67- |
| | 1.43 (m, 4H). 2H not observed (NH and OH). |
| 128 | MS m/z 473.2 [M + H]+; 1H NMR (400 MHz, Methanol-d₄) δ 7.91-7.75 |
| | (m, 4H), 6.98 (t, J = 3.4 Hz, 1H), 6.53 (d, J = 3.8 Hz, 1H), 4.44-4.34 (m, |
| | 1H), 4.10-3.95 (m, 1H), 3.86 (q, J = 11.0 Hz, 2H), 2.37 (d, J = 13.0 Hz, |
| | 1H), 2.21-2.10 (m, 1H), 2.07-1.57 (m, 8H). 2H not observed (NH and |
| | OH). |
| 129 | MS m/z 473.2 [M + H]+; 1H NMR (400 MHz, Methanol-d₄) δ 7.92-7.88 |
| | (m, 2H), 7.85-7.81 (m, 1H), 7.79-7.75 (m, 1H), 6.98 (t, J = 3.4 Hz, 1H), |
| | 6.53 (d, J = 3.8 Hz, 1H), 4.65-4.55 (m, 1H), 4.12-4.04 (m, 1H), 3.86 (q, |
| | J = 11.0 Hz, 2H), 2.29-2.20 (m, 3H), 2.05-1.94 (m, 1H), 1.92-1.78 (m, |
| | 2H), 1.77-1.60 (m, 3H), 1.54-1.41 (m, 1H). 2H not observed (NH and |
| | OH). |
| 130 | MS m/z 411.2 [M + H]+. 1H NMR (400 MHz, Methanol-d4) δ 7.68 (t, J = |
| | 3.7 Hz, 1H), 7.45 (d, J = 8.5 Hz, 1H), 7.33 (d, J = 2.3 Hz, 1H), 7.28 (dd, J = |
| | 8.5, 2.4 Hz, 1H), 6.74 (d, J = 3.4 Hz, 1H), 4.23 (quint., J = 8.1 Hz, 1H), 3.93 |
| | (s, 3H), 2.73-2.55 (m, 2H), 2.32-2.18 (m, 2H), 1.43 (s, 3H). 2H not |
| | observed (NH and OH). |
| 131 | MS m/z 436.3 [M + H]+; 1H NMR (400 MHz, Methanol-d₄) δ 7.59 (s, 1H), |
| | 7.55-7.52 (m, 1H), 7.09 (dd, J = 8.6, 2.1 Hz, 1H), 7.01 (s, 1H), 6.38 (m, |
| | 1H), 4.50-4.44 (m, 1H), 3.91 (s, 3H), 3.01-2.87 (m, 1H), 2.60-2.41 (m, |
| | 5H), 2.33 (s, 3H), 2.17-2.06 (m, 1H), 2.02-1.89 (m, 1H), 1.88-1.75 (m, |
| | 1H), 1.74-1.57 (m, 1H), 1.28 (m, 1H). NH peak not observed. |
| 132 | MS m/z 459.2 [M + H]+; 1H NMR (400 MHz, Methanol-d₄) δ 8.54-8.44 (br |
| | s, 1H, formate CH), 8.16 (s, 1H), 8.10 (d, J = 7.9 Hz, 1H), 7.81 (d, J = 8.1 |
| | Hz, 1H), 7.67 (s, 1H), 6.24 (m, 1H), 4.19-4.06 (m, 1H), 3.70-3.57 (m, |
| | 1H), 2.31 (s, 3H), 2.29-2.19 (m, 1H), 2.17-2.07 (m, 1H), 1.88-1.70 (m, |
| | 2H), 1.54-1.32 (m, 2H), 1.32-1.22 (m, 2H). NH and OH peak not |
| | observed. |
| 133 | MS m/z 437.3 [M + H]+; 1H NMR (400 MHz, Methanol-d₄) δ 7.62 (s, 1H), |
| | 7.54 (d, J = 8.5 Hz, 1H), 7.09 (dd, J = 8.6, 2.3 Hz, 1H), 7.04-6.96 (m, 1H), |
| | 6.42-6.31 (m, 1H), 4.15-4.05 (m, 1H), 3.91 (s, 3H), 3.68-3.58 (m, 1H), |
| | 2.33 (s, 3H), 2.23 (m, 1H), 2.14-2.07 (m, 1H), 1.85-1.74 (m, 2H), 1.52- |
| | 1.36 (m, 4H). NH and OH peak not observed. |
| 134 | MS m/z 488.2 [M + H]+; 1H NMR (400 MHz, Methanol-d₄) δ 8.60-8.49 |
| | (br, 1H, formate CH), 8.17 (s, 1H), 8.14-8.05 (m, 1H), 7.85-7.78 (m, |
| | 1H), 7.71 (s, 1H), 6.27 (s, 1H), 4.66-4.57 (m, 1H), 4.28-4.18 (m, 1H), |
| | 2.91-2.81 (m, 1H), 2.78-2.60 (m, 4H), 2.32 (s, 3H), 2.05-1.87 (m, 3H), |
| | 1.21-1.15 (m, 3H). NH and OH peak not observed. |
| 135 | MS m/z 466.2 [M + H]+; 1H NMR (400 MHz, Methanol-d₄) δ 7.66-7.62 (m, |
| | 1H), 7.58-7.47 (m, 1H), 7.16-7.06 (m, 1H), 7.05-6.96 (m, 1H), 6.39- |
| | 6.36 (m, 1H), 4.66-4.55 (m, 1H), 4.29-4.18 (m, 1H), 3.91 (s, 3H), 2.97- |
| | 2.86 (m, 2H), 2.86-2.72 (m, 4H), 2.34 (s, 3H), 2.00-1.93 (m, 2H), 1.21 (t, |
| | J = 7.2 Hz, 3H). NH and OH peak not observed. |
| 136 | MS m/z 389.3 [M + H]+; 1H NMR (400 MHz, Methanol-d₄) δ = 7.85 (s, 1H), |
| | 7.44 (dd, J = 2.3, 7.7 Hz, 1H), 7.29-7.13 (m, 2H), 6.98-6.47 (m, 3H), 4.21- |
| | 4.08 (m, 1H), 3.73-3.60 (m, 1H), 2.47 (s, 3H), 2.33-2.20 (m, 1H), 2.18- |
| | 2.06 (m, 1H), 1.90-1.73 (m, 2H), 1.57-1.34 (m, 4H); 2H not observed |
| | (NH and OH). |
| 137 | MS m/z 389.3 [M + H]+; 1H NMR (400 MHz, Methanol-d₄) δ 7.93-7.87 (m, |
| | 1H), 7.44 (d, J = 7.8 Hz, 1H), 7.27-7.16 (m, 2H), 6.98-6.56 (m, 2H), 6.55- |
| | 6.48 (m, 1H), 4.36-4.22 (m, 2H), 2.48 (s, 3H), 2.07-1.80 (m, 4H), 1.79- |
| | 1.63 (m, 2H), 1.58-1.44 (m, 2H); 2H not observed (NH and OH). |
| 138 | MS m/z 421.2 [M + H]+; 1H NMR (500 MHz, Methanol-d₄) δ 7.91-7.88 |
| | (m, 1H), 7.48 (d, J = 8.4 Hz, 1H), 7.11 (s, 1H), 7.08 (d, J = 8.4 Hz, 1H), |
| | 6.98 (t, J = 3.3 Hz, 1H), 6.56 (d, J = 3.8 Hz, 1H), 4.16-4.08 (m, 1H), 3.89 |
| | (s, 3H), 3.75-3.61 (m, 3H), 2.27-2.21 (m, 1H), 2.15-2.07 (m, 1H), 1.85- |
| | 1.74 (m, 2H), 1.54-1.34 (m, 4H). 2H not observed (NH and OH). |
| 139 | MS m/z 406.2 [M + H]+; 1H NMR (500 MHz, Methanol-d₄) δ 7.81-7.78 |
| | (m, 1H), 7.47 (d, J = 8.5 Hz, 1H), 7.11 (s, 1H), 7.07 (dd, J = 8.5, 2.2 Hz, |
| | 1H), 6.98 (t, J = 3.3 Hz, 1H), 6.55 (d, J = 3.8 Hz, 1H), 5.03 (t, J = 9.5 Hz, |
| | 1H), 3.89 (s, 3H), 3.67 (q, J = 11.0 Hz, 2H), 3.53-3.44 (m, 2H), 2.77- |
| | 2.71 (m, 1H), 2.37-2.29 (m, 1H). 2H not observed (2 NH). |
| 140 | MS m/z 460.1 [M + H]+; 1H NMR (400 MHz, DMSO-d₆) δ 8.01 (d, J = 2.0 |
| | Hz, 1H), 7.98-7.93 (m, 2H), 7.92 (s, 1H), 7.37 (d, J = 7.8 Hz, 1H), 6.99- |
| | 6.96 (m, 1H), 6.51 (d, J = 3.3 Hz, 1H), 4.32 (s, 1H), 3.07 (d, J = 7.1 Hz, |
| | 1H), 2.75-2.67 (m, 1H), 2.22 (s, 3H), 2.02 (d, J = 9.5 Hz, 1H), 1.92 (dd, |
| | J = 23.8, 10.7 Hz, 2H), 1.76 (d, J = 12.9 Hz, 1H), 1.60 (d, J = 12.6 Hz, 1H), |
| | 1.43 (dd, J = 11.5, 4.1 Hz, 1H). |
| 141 | MS m/z 447.1 [M + H]+; 1H NMR (400 MHz, Methanol-d₄) δ 7.91-7.86 |
| | (m, 2H), 7.86 (dd, J = 2.9, 1.1 Hz, 1H), 7.78 (s, 1H), 7.00-6.96 (m, 1H), |
| | 6.58-6.54 (m, 1H), 4.60 (t, J = 8.1 Hz, 1H), 3.59 (d, J = 6.1 Hz, 2H), 2.63 |
| | (ddd, J = 16.6, 7.6, 2.8 Hz, 2H), 2.36-2.27 (m, 1H), 1.95 (dt, J = 11.8, 9.3 |
| | Hz, 2H). NH and OH not observed. |
| 142 | MS m/z 483.0 [M + H]+; 1H NMR (400 MHz, DMSO-d₆) δ 9.33 (s, 1H), |
| | 8.15 (dd, J = 2.9, 1.1 Hz, 1H), 7.99-7.90 (m, 4H), 7.13 (t, J = 7.8 Hz, 1H), |
| | 7.01 (dd, J = 3.9, 2.9 Hz, 1H), 6.89 (dd, J = 9.7, 4.9 Hz, 2H), 6.63 (dd, J = |
| | 8.0, 1.6 Hz, 1H), 6.52 (dd, J = 3.9, 1.0 Hz, 1H), 5.49 (p, J = 7.0 Hz, 1H), |
| | 1.61 (d, J = 7.0 Hz, 3H). |
| 143 | MS m/z 424.4 [M + H]+; 1H NMR (400 MHz, Methanol-d₄) δ 7.76 (s, 1H), |
| | 7.29 (d, J = 8.4 Hz, 1H), 7.01 (d, J = 2 Hz, 1H), 6.88 (t, J = 3.2 Hz, 1H), |
| | 6.66 (dd, J = 2.4 Hz, J = 8.4 Hz, 1H), 7.04 (d, J = 2.8 Hz, 1H), 4.43(s, 1H), |
| | 3.87(s, 3H), 3.78-3.67 (m, 3H), 3.13-3.12 (m, 1H), 2.69(S, 1H), 2.63- |
| | 2.53 (m, 2H), 2.39(S, 2H), 2.02-2.00 (m, 1H), 1.85-1.83 (m, 1H), 1.74- |
| | 1.67 (m, 2H), 0.74-0.69 (m, 2H), 0.59-0.55 (m, 2H). |
| 144 | MS m/z 423.3 [M + H]+; 1H NMR (500 MHz, Methanol-d₄) δ 7.90 (d, J = |
| | 1.7 Hz, 1H), 7.47 (d, J = 8.4 Hz, 1H), 7.11 (s, 1H), 7.08 (dd, J = 8.5, 2.4 Hz, |
| | 1H), 6.99 (t, J = 3.4 Hz, 1H), 6.57 (d, J = 3.8 Hz, 1H), 4.30 (td, J = 10.7, 3.2 |
| | Hz, 1H), 4.03 (dd, J = 11.1, 4.9 Hz, 1H), 3.97 (dd, J = 11.7, 4.1 Hz, 1H), |
| | 3.89 (s, 3H), 3.77 (td, J = 9.7, 4.9 Hz, 1H), 3.73-3.63 (m, 2H), 3.55 (t, J = |
| | 11.1 Hz, 1H), 3.26 (t, J = 11.1 Hz, 1H), 2.26 (dd, J = 13.1, 4.1 Hz, 1H), 1.79 |
| | (qd, J = 12.2, 4.6 Hz, 1H). 2H not observed (NH and OH). |
| 145 | MS m/z 420.4 [M + H]+; 1H NMR (500 MHz, Methanol-d₄) δ 8.76 (brs, 1 H, |
| | formic acid), 7.85 (s, 1H), 7.46 (d, J = 8.5 Hz, 1H), 7.12 (s, 1H), 7.08 (dd, |
| | J = 8.5, 2.3 Hz, 1H), 6.99 (t, J = 3.2 Hz, 1H), 6.56 (d, J = 3.8 Hz, 1H), 4.58- |
| | 4.51 (m, 1H), 3.89 (s, 3 H), 3.81-3.73 (m, 1H), 3.67 (q, J = 11.0 Hz, 2H), |
| | 3.44-3.34 (m, 1H), 3.11-2.95 (m, 2H), 2.87 (s, 3H), 2.30-2.11 (m, 2H), |
| | 2.01-1.80 (m, 2H). 1H not observed (NH). |
| 146 | MS m/z 472.3 [M + H]+; 1H NMR (400 MHz, Methanol-d₄) δ 7.92 (s, 1H), |
| | 7.85 (d, J = 8.6 Hz, 1H), 7.82-7.74 (m, 2H), 7.03 (t, J = 3.3 Hz, 1H), 6.58 |
| | (d, J = 3.9 Hz, 1H), 3.91-3.74 (m, 2H), 3.72-3.63 (m, 2H), 3.61-3.51 |
| | (m, 1H), 3.46-3.34 (m, 1H), 3.08-2.89 (m, 2H), 2.84 (s, 3H), 2.53-2.37 |
| | (m, 1H), 2.11-1.97 (m, 2H), 1.91-1.73 (m, 1H), 1.51-1.33 (m, 1H). 1H |
| | not observed (NH). |
| 147 | MS m/z 472.3 [M + H]+; 1H NMR (400 MHz, Methanol-d₄) δ 7.92 (s, 1H), |
| | 7.85 (d, J = 8.6 Hz, 1H), 7.82-7.74 (m, 2H), 7.03 (t, J = 3.3 Hz, 1H), 6.58 |
| | (d, J = 3.9 Hz, 1H), 3.91-3.74 (m, 2H), 3.72-3.63 (m, 2H), 3.61-3.51 |
| | (m, 1H), 3.46-3.34 (m, 1H), 3.08-2.89 (m, 2H), 2.84 (s, 3H), 2.53-2.37 |
| | (m, 1H), 2.11-1.97 (m, 2H), 1.91-1.73 (m, 1H), 1.51-1.33 (m, 1H). 1H |
| | not observed (NH). |
| 148 | MS m/z 461.2 [M + H]+; 1H NMR (400 MHz, Methanol-d4) δ 7.80 (t, J = |
| | 3.6 Hz, 1H), 7.44 (d, J = 8.7 Hz, 1H), 7.32 (s, 1H), 7.28-7.20 (m, 2H), 7.09- |
| | 7.01 (m, 2H), 6.80 (d, J = 9.0 Hz, 1H), 6.76 (d, J = 3.3 Hz, 1H), 5.48 (d, |
| | J = 7.1 Hz, 1H), 3.92 (s, 3H), 3.78 (s, 3H), 1.68 (d, J = 7.0 Hz, 3H). 1H not |
| | observed (NH). |
| 149 | MS m/z 447.2 [M + H]+; 1H NMR (400 MHz, Methanol-d4) δ 7.81 (t, J = |
| | 3.6 Hz, 1H), 7.44 (d, J = 8.3 Hz, 1H), 7.32 (s, 1H), 7.26 (d, J = 8.3 Hz, 1H), |
| | 7.14 (t, J = 7.8 Hz, 1H), 6.97-6.89 (m, 2H), 6.76 (d, J = 3.3 Hz, 1H), 6.66 |
| | (d, J = 8.2 Hz, 1H), 5.45 (q, J = 6.7 Hz, 1H), 3.92 (s, 3H), 1.67 (d, J = 7.0 |
| | Hz, 3H). NH and OH not observed. |
| 150 | MS m/z 429.0 [M + H]+; 1H NMR (400 MHz, DMSO-d₆) δ 8.04-8.03 (m, |
| | 1H), 7.94 (d, J = 8.0 Hz, 1H), 7.88 (d, J = 8.0 Hz, 1H), 7.80 (s, 1H), 7.61 (d, |
| | J = 2.4 Hz, 1H), 6.86-6.84 (m, 1H), 6.57 (dd, J = 1.2 3.6 Hz, 1H), 6.21 (s, |
| | 1H). NH not observed. |
| 151 | MS m/z 461.1 [M + H]+; 1H NMR (400 MHz, DMSO-d₆) δ 8.02-7.99 (m, |
| | 1H), 7.98-7.93 (m, 2H), 7.92 (s, 1H), 7.41 (d, J = 7.8 Hz, 1H), 6.97 (dd, |
| | J = 3.8, 2.9 Hz, 1H), 6.49 (dd, J = 3.9, 1.0 Hz, 1H), 4.81 (d, J = 5.2 Hz, 1H), |
| | 4.19-3.98 (m, 1H), 3.65-3.48 (m, 1H), 2.17-2.05 (m, 1H), 2.03-1.94 |
| | (m, 1H), 1.80-1.64 (m, 2H), 1.42-1.23 (m, 4H). |
| 152 | MS m/z 445.1 [M + H]+; 1H NMR (400 MHz, DMSO-d₆) δ 7.85 (dd, J = 2.8, |
| | 0.8 Hz, 1H), 7.69 (d, J = 8.4 Hz, 1H), 7.33 (dd, J = 8.4, 2.4 Hz, 1H), 7.29 (s, |
| | 1H), 7.03 (t, J = 73.2 Hz, 1H), 6.98 (t, J = 3.6 Hz, 1H), 6.56 (d, J = 3.2 Hz, |
| | 1H), 4.39-4.37 (m, 1H), 4.19-4.15 (m, 1H), 3.89-3.86 (m, 1H), 3.52-3.42 |
| | (m, 2H), 2.22 (s, 1H), 1.86-1.79 (m, 3H). NH not observed. |
| 153 | MS m/z 459.0 [M + H]+; 1H NMR (400 MHz, DMSO-d₆) δ 7.83 (d, J = 2.4 |
| | Hz, 1H), 7.70 (d, J = 8.0 Hz, 1H), 7.33 (dd, J = 8.4, 3.0 Hz, 1H), 7.29 (s, |
| | 1H), 7.03 (t, J = 73.2 Hz, 1H), 6.97 (dd, J = 4.0, 3.2 Hz, 1H), 6.55 (d, J = 4.0 |
| | Hz, 1H), 4.27-4.21 (m, 1H), 3.76-3.71 (m, 1H), 2.43 (d, J = 11.6 Hz, 1H), |
| | 2.13 (d, J = 11.6 Hz, 1H), 1.99 (d, J = 12.4 Hz, 1H), 1.91-1.86 (m, 1H), 1.50- |
| | 1.37 (m, 3H), 1.31-1.259 (m, 1H). NH and OH not observed. |
| 154 | MS m/z 444.0 [M + H]+; 1H NMR (400 MHz, DMSO-d₆) δ 7.79 (s, 1H), |
| | 7.69 (d, J = 8.4 Hz, 1H), 7.34-7.29 (m, 2H), 7.04 (t, J = 73.2 Hz, 1H), 6.98 (s, |
| | 1H), 6.57 (s, 1H), 5.07-5.03 (m, 1H), 3.51-3.46 (m, 2H), 2.73-2.72 (m, 1H), |
| | 2.36-2.31 (m, 1H). NH not observed. |
| 155 | MS m/z 427.0 [M + H]+; 1H NMR (400 MHz, DMSO-d₆) δ 8.01 (s, 1H), |
| | 7.73 (d, J = 8.4 Hz, 1H), 7.60 (s, 1H), 7.33 (d, J = 8.8 Hz, 1H), 7.30 (s, 1H), |
| | 7.04 (t, J = 73.2 Hz, 1H), 6.83 (m, 1H), 6.54 (d, J = 2.8 Hz, 1H), 6.20 (s, |
| | 1H). NH not observed. |
| 156 | MS m/z 405.3 [M + H]+; 1H NMR (400 MHz, Methanol-d₄) δ 7.89 (d, J = |
| | 2.4 Hz, 1H), 7.44-7.37 (m, 2H), 7.33 (d, J = 7.9 Hz, 1H), 6.97 (t, J = 3.3 |
| | Hz, 1H), 6.53 (d, J = 3.8 Hz, 1H), 4.18-4.07 (m, 1H), 3.74-3.58 (m, 3H), |
| | 2.45 (s, 3H), 2.30-2.20 (m, 1H), 2.17-2.06 (m, 1H), 1.86-1.74 (m, 2H), |
| | 1.53-1.34 (m, 4H). 2H not observed (NH and OH). |
| 157 | MS m/z 390.2 [M + H]+; 1H NMR (400 MHz, Methanol-d₄) δ 7.80 (d, J = |
| | 2.6 Hz, 1H), 7.44-7.37 (m, 2H), 7.33 (d, J = 7.9 Hz, 1H), 6.98 (t, J = 3.1 |
| | Hz, 1H), 6.53 (d, J = 3.9 Hz, 1H), 5.04 (t, J = 9.5 Hz, 1H), 3.65 (q, J = 11.1 |
| | Hz, 2H), 3.55-3.44 (m, 2H), 2.79-2.71 (m, 1H), 2.45 (s, 3H), 2.40-2.28 |
| | (m, 1H). 2 H not observed (2 NH). |
| 158 | MS m/z 391.1 [M + H]+; 1H NMR (400 MHz, Methanol-d₄) δ 7.86 (d, J = |
| | 2.5 Hz, 1H), 7.44-7.37 (m, 2H), 7.33 (d, J = 7.9 Hz, 1H), 6.97 (t, J = 3.3 |
| | Hz, 1H), 6.54 (d, J = 3.8 Hz, 1H), 4.26 (quint, J = 8.0 Hz, 1H), 3.67 (q, J = |
| | 11.1 Hz, 2H), 2.70-2.62 (m, 2H), 2.45 (s, 3H), 2.32-2.23 (m, 2H), 1.44 |
| | (s, 3H). 2H not observed (NH and OH). |
| 159 | MS m/z 404.3 [M + H]+; 1H NMR (400 MHz, Methanol-d₄) δ 8.03-7.95 |
| | (m, 1H), 7.47-7.41 (m, 2H), 7.39 (d, J = 7.9 Hz, 1H), 7.13-7.07 (m, 1H), |
| | 6.78-6.73 (m, 1H), 4.61-4.49 (m, 1H), 4.04-3.94 (m, 1H), 3.69-3.53 |
| | (m, 3H), 3.10-2.83 (m, 5H), 2.47 (s, 3H), 2.39-2.09 (m, 2H), 2.09-1.89 |
| | (m, 1H), 1.89-1.75 (m, 1H). 1H not observed (NH). |
| 160 | MS m/z 459.2 [M + H]+; 1H NMR (400 MHz, Methanol-d₄) δ 8.20-8.13 |
| | (m, 1H), 8.13-8.06 (m, 1H), 7.87-7.78 (m, 1H), 7.72-7.62 (m, 1H), 6.24 |
| | (s, 1H), 4.18-4.06 (m, 1H), 3.72-3.58 (m, 1H), 2.31 (s, 3H), 2.29-2.18 |
| | (m, 1H), 2.15-2.06 (m, 1H), 1.86-1.71 (m, 3H), 1.54-1.32 (m, 3H). NH |
| | and OH peak not observed. |
| 161 | MS m/z 444.2 [M + H]+; 1H NMR (400 MHz, Methanol-d₄) δ 8.22-8.14 |
| | (m, 1H), 8.13-8.07 (m, 1H), 7.82 (d, J = 8.0 Hz, 1H), 7.59 (s, 1H), 6.27 |
| | (m, 1H), 5.13-4.97 (m, 1H), 3.57-3.43 (m, 1H), 2.85-2.67 (m, 2H), 2.40- |
| | 2.21 (m, 4H). NH peaks were not observed. |
| 162 | MS m/z 422.3 [M + H]+; 1H NMR (400 MHz, Methanol-d₄) δ 7.61-7.48 |
| | (m, 2H), 7.16-7.05 (m, 1H), 7.04-6.92 (m, 1H), 6.43-6.30 (m, 1H), 5.07- |
| | 4.95 (m, 1H), 3.91 (s, 3H), 3.56-3.40 (m, 2H), 2.81-2.68 (m, 1H), 2.33 |
| | (s, 4H), NH peak not observed. |
| 163 | MS m/z 437.3 [M + H]+; 1H NMR (500 MHz, Methanol-d₄) δ 7.62 (s, 1H), |
| | 7.53 (d, J = 8.5 Hz, 1H), 7.08 (d, J = 8.6, 1H), 7.00 (s, 1H), 6.36 (s, 1H), |
| | 4.17-4.06 (m, 1H), 3.90 (s, 3H), 3.70-3.57 (m, 1H), 2.32 (s, 3H), 2.27- |
| | 2.20 (m, 1H), 2.15-2.05 (m, 1H), 1.86-1.72 (m, 2H), 1.49-1.37 (m, 4H), |
| | NH and OH peak not observed. |
| 164 | MS m/z 461.2 [M + H]+; 1H NMR (500 MHz, Methanol-d₄) δ 8.24 (s, 1H), |
| | 8.18 (d, J = 7.9 Hz, 1H), 8.02 (s, 1H), 7.92 (d, J = 8.1 Hz, 1H), 6.66 (m, |
| | 1H), 4.22-4.12 (m, 1H), 4.07-3.97 (m, 2H), 3.80-3.72 (m, 1H), 3.59- |
| | 3.50 (m, 1H), 3.29-3.22 (m, 1H), 2.36 (s, 3H), 2.22-2.11 (m, 1H), 1.97- |
| | 1.83 (m, 1H). NH and OH peak not observed. |
| 165 | MS m/z 439.2 [M + H]+; 1H NMR (500 MHz, Methanol-d₄) δ 8.00 (s, 1H), |
| | 7.68 (d, J = 8.7 Hz, 1H), 7.23-7.16 (m, 1H), 7.12 (s, 1H), 6.94-6.85 (m, |
| | 1H), 4.27-4.16 (m, 1H), 4.09-4.02 (m, 1H), 3.95 (s, 3H), 3.81-3.72 (m, |
| | 1H), 3.57-3.48 (m, 1H), 3.27-3.19 (m, 2H), 2.40 (s, 3H), 2.22-2.16 (m, |
| | 1H), 1.90-1.77 (m, 1H). NH and OH Peak not observed. |
| 166 | MS m/z 418.3 [M + H]+; 1H NMR (400 MHz, Methanol-d₄) δ 7.81 (d, J = |
| | 2.3 Hz, 1H), 7.48 (d, J = 8.5 Hz, 1H), 7.01-6.57 (m, 4H), 6.57-6.51 (m, |
| | 1H), 4.57-4.40 (m, 1H), 3.90 (s, 3H), 3.29-3.17 (m, 1H), 2.86-2.73 (m, |
| | 1H), 2.61-2.47 (m, 2H), 2.35-2.20 (m, 2H), 2.18-2.07 (m, 1H), 1.94- |
| | 1.57 (m, 3H), 1.16 (t, J = 7.2 Hz, 3H). NH not observed |
| 167 | MS m/z 407.3 [M + H]+; 1H NMR (400 MHz, Methanol-d₄) δ 7.90-7.81 (m, |
| | 1H), 7.49 (d, J = 8.5 Hz, 1H), 7.03-6.58 (m, 4H), 6.58-6.53 (m, 1H), 4.41- |
| | 4.25 (m, 1H), 4.09-3.95 (m, 2H), 3.91 (s, 3H), 3.86-3.69 (m, 1H), 3.57 |
| | (t, J = 11.8 Hz, 1H), 3.31-3.22 (m, 1H), 2.33-2.22 (m, 1H), 1.88-1.71 |
| | (m, 1H); 2H (OH and NH) not observed |
| 168 | MS m/z 459.1 [M + H]+; 1H NMR (400 MHz, Methanol-d₄) δ 7.87 (dd, J = 2.8, |
| | 0.8 Hz, 1H), 7.69 (d, J = 8.4 Hz, 1H), 7.33 (dd, J = 8.4, 2.0 Hz, 1H), 7.29 |
| | (s, 1H), 7.03 (t, J = 73.2, 1H), 6.98-6.96 (m, 1H), 6.54 (d, J = 3.2 Hz, 1H), |
| | 4.17-4.13 (m, 1H), 3.67-3.62 (m, 1H), 2.27-2.21(m, 1H), 2.14-2.01 |
| | (m, 1H), 1.83-1.77 (m, 2H), 1.48-1.40 (m, 4H). 2H not observed (NH |
| | and OH). |
| 169 | MS m/z 438.1 [M + H]+; 1H NMR (400 MHz, Methanol-d₄) δ 7.82 (d, J = |
| | 2.8 Hz, 1H), 7.69 (d, J = 8.0 Hz, 1H), 7.62-7.56 (m, 2H), 6.95 (t, J = 3.2 |
| | Hz, 1H), 6.52 (d, J = 4.0 Hz, 1H), 5.80 (s, 1H), 5.67 (s, 1H), 4.53-4.43 (m, |
| | 1H), 3.26-3.18 (m, 1H), 2.84-2.75 (m, 1H), 2.60-2.45 (m, 2H), 2.33- |
| | 2.19 (m, 2H), 2.16-2.06 (m, 1H), 1.91-1.82 (m, 1H), 1.81-1.70 (m, 1H), |
| | 1.69-1.59 (m, 1H), 1.15 (t, J = 7.2 Hz, 3H). 1H not observed (NH). |
| 170 | MS m/z 474.1 [M + H]+; 1H NMR (400 MHz, Methanol-d₄) δ 7.91 (dd, J = |
| | 3.2, 1.2 Hz, 1H), 7.68 (d, J = 8.4 Hz, 1H), 7.33 (dd, J = 8.8, 2.4 Hz, 1H), |
| | 7.29 (s, 1H), 7.03 (t, J = 73.2, 1H), 6.99-6.98 (m, 1H), 6.56 (dd, J = 4.0, |
| | 0.8 Hz, 1H), 4.65-4.56 (m, 1H), 4.27-4.17 (m, 1H), 2.91-2.71 (m, 2H), |
| | 2.67-2.50 (m, 2H), 2.38 (s, 3H), 1.93-1.90 (m, 2H). 2H not observed (NH |
| | and OH). |
| 171 | MS m/z 488.1 [M + H]+; 1H NMR (400 MHz, Methanol-d₄) δ 7.91 (dd, J = |
| | 3.2, 1.2 Hz, 1H), 7.69 (d, J = 8.4 Hz, 1H), 7.34 (dd, J = 8.4, 2.4 Hz, 1H), |
| | 7.30 (s, 1H), 7.03 (t, J = 73.2, 1H), 7.00 (t, J = 3.2, 1H), 6.58 (dd, J = 4.0 1.2 |
| | Hz, 1H), 4.66-4.63 (m, 1H), 4.28-4.26 (m, 1H), 3.13-3.05 (m, 1H), 2.95- |
| | 2.88 (m, 1H), 2.81-2.74 (m, 4H), 2.00-1.97 (m, 2H), 1.23 (t, J = 7.2, |
| | 3H). 2H not observed (NH and OH). |
| 172 | MS m/z 481.1 [M + H]+; 1H NMR (400 MHz, Methanol-d₄) δ 7.97 (dd, J = |
| | 3.2, 1.2 Hz, 1H), 7.66 (d, J = 7.6 Hz, 1H), 7.31 (dd, J = 8.8, 2.4 Hz, 1H), |
| | 7.27 (s, 1H), 7.14 (t, J = 8.0 Hz, 1H), 7.02 (t, J = 73.2 Hz, 1H), 6.99-6.98 |
| | (m, 1H), 6.95 (d, J = 7.6 Hz, 1H), 6.92-6.91 (m, 1H), 6.65 (dd, J = 7.2, 1.6 |
| | Hz, 1H), 6.55 (dd, J = 4.0, 1.2 Hz, 1H), 5.51-5.46 (m, 1H), 1.68 (d, J = 6.8 |
| | Hz, 3H). 2H not observed (NH and OH). |
| 173 | MS m/z 454.1 [M + H]+; 1H NMR (400 MHz, DMSO-d₆) δ 7.95 (s, 1H), |
| | 7.71 (d, J = 8.0 Hz, 1H), 7.64-7.62 (m, 2H), 7.25 (d, J = 8.0 Hz, 1H), 6.95- |
| | 6.91 (m, 1H), 6.41 (d, J = 4.0 Hz, 1H), 5.98 (s, 1H), 5.84 (s, 1H), 4.41- |
| | 4.30 (m, 2H), 3.55-3.49 (m, 2H), 3.17-3.15 (m, 1H), 2.84-3.81 (m, 1H), |
| | 2.47-2.44 (m, 2H), 2.10-2.00 (m, 3H), 1.78-1.69 (m, 1H), 1.60-1.57 |
| | (m, 1H), 1.47-1.44 (m, 1H). |
| 174 | MS m/z 390.3 [M + H]+; 1H NMR (500 MHz, Methanol-d₄) δ 7.77 (s, 1H), |
| | 7.49 (d, J = 8.5 Hz, 1H), 7.01-6.94 (m, 2H), 6.94-6.61 (m, 2H), 6.61- |
| | 6.52 (m, 1H), 5.12-5.00 (m, 1H), 3.91 (s, 3H), 3.56-3.44 (m, 2H), 2.81- |
| | 2.69 (m, 1H), 2.40-2.28 (m, 1H); 2H (NH) not observed |
| 175 | MS m/z 434.4 [M + H]+; 1H NMR (500 MHz, Methanol-d₄) δ 7.87 (s, 1H), |
| | 7.48 (d, J = 8.4 Hz, 1H), 7.03-6.60 (m, 4H), 6.58-6.53 (m, 1H), 4.72- |
| | 4.52 (m, 1H), 4.33-4.15 (m, 1H), 3.91 (s, 3H), 3.00-2.42 (m, 6H), 2.05- |
| | 1.82 (m, 2H), 1.17 (t, J = 7.2 Hz, 3H); 2H (NH and OH) not observed. |
| 176 | MS m/z 419.4 [M + H]+; 1H NMR (500 MHz, Methanol-d₄) δ 7.83-7.78 (m, |
| | 1H), 7.49 (d, J = 8.4 Hz, 1H), 7.00-6.61 (m, 4H), 6.52 (d, J = 3.7 Hz, 1H), |
| | 4.28-4.17 (m, 1H), 3.91 (s, 3H), 3.50-3.43 (m, 2H), 2.34-2.18 (m, 2H), |
| | 1.98-1.80 (m, 2H), 1.79-1.67 (m, 1H), 1.62-1.35 (m, 2H), 1.23-1.10 |
| | (m, 1H), 1.07-0.94 (m, 1H); 2H (NH and OH) not observed. |
| 177 | MS m/z 405.4 [M + H]+; 1H NMR (500 MHz, Methanol-d₄) δ 7.87-7.82 (m, |
| | 1H), 7.48 (d, J = 8.5 Hz, 1H), 7.00-6.93 (m, 2H), 6.93-6.61 (m, 2H), 6.57- |
| | 6.50 (m, 1H), 4.20-4.09 (m, 1H), 3.91 (s, 3H), 3.72-3.60 (m, 1H), 2.36- |
| | 2.19 (m, 1H), 2.18-2.06 (m, 1H), 1.90-1.71 (m, 2H), 1.53-1.38 (m, 4H); |
| | 2H (NH and OH) not observed. |
| 178 | MS m/z 446.2 [M + H]+; 1H NMR (400 MHz, Methanol-d₄) δ 7.80-7.77 |
| | (m, 1H), 7.72 (s, 1H), 7.57 (d, J = 8.0 Hz, 1H), 7.41 (d, J = 8.4 Hz, 1H), 6.95- |
| | 6.90 (m, 1H), 6.43 (d, J = 3.6 Hz, 1H), 4.48 (s, 1H), 3.93-3.84 (m, 1H), |
| | 3.28-3.17 (m, 1H), 2.78-2.81(m, 1H), 2.53-2.55 (m, 2H), 2.26-2.28 (m, |
| | 2H), 2.08-2.10 (m, 1H), 1.84-1.86 (m, 1H), 1.76-1.78 (m, 1H), 1.59-1.61 |
| | (m, 1H), 1.16 (t, J = 7.2 Hz, 3H), 0.76-0.83 (m, 2H), 0.61-0.63 (m, 2H). 1H |
| | not observed (NH). |
| 179 | MS m/z 462.1 [M + H]+; 1H NMR (400 MHz, Methanol-d₄) δ 7.82 (dd, J = |
| | 2.8, 1.2 Hz, 1H), 7.72 (s, 1H), 7.57 (d, J = 8.0 Hz, 1H), 7.41 (d, J = 8.0 Hz, |
| | 1H), 6.95-6.90 (m, 1H), 6.43 (dd, J = 4.0, 1.2 Hz, 1H), 4.47 (s, 1H), 3.93- |
| | 3.84 (m, 1H), 3.70-3.74(m, 2H), 3.19-3.21(m, 1H), 2.73-2.75 (m, 1H), |
| | 2.61-2.65(m, 2H), 2.42-2.44 (m, 2H), 2.03-2.05 (m, 1H), 1.86-1.91(m, |
| | 1H), 1.72-1.77(m, 2H), 0.76-0.81(m, 2H), 0.61-0.64(m, 2H). 2H not |
| | observed (NH and OH). |
| 180 | MS m/z 437.1 [M + H]+; 1H NMR (400 MHz, Methanol-d₄) δ 7.95 (dd, J = |
| | 1.2, 2.8 Hz, 1H), 7.54 (d, J = 8.4 Hz, 1H), 7.09 (dd, J = 2.4, 8.8 Hz, 1H), |
| | 6.97-7.01 (m, 2H), 6.56 (d, J = 3.2, 1H), 4.37 (dd, J = 4.0, 16.0 Hz, 1H), |
| | 3.91 (s, 3H), 2.00-2.03 (m, 1H), 1.82-1.88 (m, 2H), 1.62-1.74 (m, 3H), |
| | 1.44-1.50 (m, 2H), 1.28 (s, 3H). 2H not observed (NH and OH). |
| 181 | MS m/z 437.1 [M + H]+; 1H NMR (400 MHz, Methanol-d₄) δ 7.95 (d, J = |
| | 1.6 Hz, 1H), 7.55 (d, J = 8.4 Hz, 1H), 7.09 (dd, J = 2.4, 8.8 Hz, 1H), 6.97- |
| | 7.01 (m, 2H), 6.56 (d, J = 3.6, 1H), 4.37 (dd, J = 4.0, 11.6 Hz, 1H), 3.91 (s, |
| | 3H), 2.00-2.03 (m, 1H), 1.81-1.88 (m, 2H), 1.62-1.71 (m, 3H), 1.40- |
| | 1.55 (m, 2H), 1.28 (s, 3H). 2H not observed (NH and OH). |
| 182 | MS m/z 408.2 [M + H]+; 1H NMR (400 MHz, DMSO-d₆) δ 7.85 (s, 1H), |
| | 7.27 (dd, J = 3.6 Hz, J = 8.4 Hz, 1H), 7.07 (d, J = 5.2 Hz, 1H), 6.98 (s, 1H), |
| | 6.86 (d, J = 2.8 Hz, 1H), 6.87 (t, J = 2.4 Hz, 1H), 6.27 (s, 1H), 3.94(s, 1H), |
| | 3.81(dd, J = 2.4 Hz, J = 24.4 Hz, 1H), 3.18-3.15 (m, 1H), 2.83-2.71 (s, |
| | 1H), 2.50(m, 1H), 2.39-2.37 (m, 2H), 2.04-1.99 (m, 1H), 1.92-1.86 (m, |
| | 2H), 1.77-1.74 (m, 1H), 1.91-1.53 (m, 1H), 1.47-1.38 (m, 1H), 1.07- |
| | 1.00 (m, 3H), 0.73(m, 1H), 0.52(m, 1H). 1H not observed (NH). |

### Example 2

### Preparation of Compound 2

### (R)-N-(1-methylpiperidin-3-yl)-1-(2-(2,2,2-trifluoroethyl)-4-(trifluoromethyl)phenyl)imidazo[1,5-d][1,2,4]triazin-4-amine

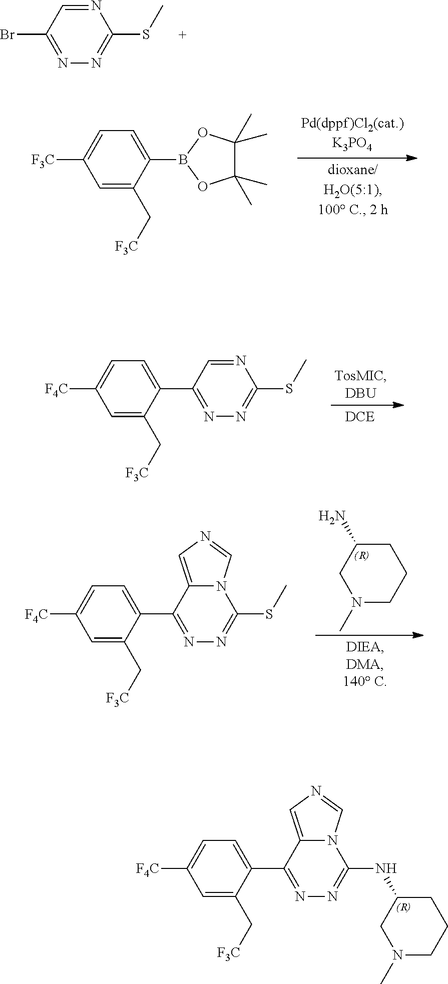

### Step 1. 3-(Methylthio)-6-(2-(2,2,2-trifluoroethyl)-4-(trifluoromethyl)phenyl)-1,2,4-triazine

To a solution of 4,4,5,5-tetramethyl-2-(2-(2,2,2-trifluoroethyl)-4-(trifluoromethyl)phenyl)-1,3,2-dioxaborolane (Intermediate 2b, 2 g, 5.7 mmol, 1.0 eq.) in 1,4-dioxane/H₂O (5:1, 24 mL) was added 6-bromo-3-(methylthio)-1,2,4-triazine (1.1 g, 5.1 mmol, 1 eq.), Pd(dppf)Cl₂ (372.6 mg, 0.1 eq.) and K₃PO₄ (2.16 g, 2 eq.) under N₂ atmosphere at rt. The reaction mixture was stirred for 2 h at 90° C. under N₂, then diluted with water (20 mL) and extracted with EtOAc (50 mL×3). The combined organic layers were washed with brine, dried over anhydrous Na₂SO₄ and concentrated. The residue was purified by flash chromatography on silica gel (PE:EA=0%˜10%) to afford 3-(methylthio)-6-(2-(2,2,2-trifluoroethyl)-4-(trifluoromethyl)phenyl)-1,2,4-triazine (1.6 g, 81.1% yield) as yellow oil. ¹H NMR (400 MHz, DMSO-d₆) δ 8.95 (s, 1H), 8.03-7.97 (m, 2H), 7.89 (d, J=8.0 Hz, 1H), 4.10 (q, J=11.2 Hz, 2H), 2.70 (s, 3H).

### Step 2. 4-(Methylthio)-1-(2-(2,2,2-trifluoroethyl)-4-(trifluoromethyl)phenyl)imidazo[1,5-d][1,2,4]triazine

To a solution of TosMIC (1.1 g, 5.4 mmol, 1.2 eq.) and DBU (827.4 mg, 5.4 mmol, 1.2 eq.) in DCE (20 mL) was added 3-(methylthio)-6-(2-(2,2,2-trifluoroethyl)-4-(trifluoromethyl)phenyl)-1,2,4-triazine (1.6 g, 4.5 mmol, 1.0 eq.) under N₂ at rt. The reaction mixture was stirred for 2 h at rt, then diluted with water (50 mL) and extracted with EtOAc (50 mL×3). The combined organic layers were washed with brine, dried over anhydrous Na₂SO₄ and concentrated. The residue was purified by flash chromatography on silica gel (PE:EA=0% 25%) to afford title product (1.29 g, 44.9% yield) as yellow oil. MS m/z 392.9 [M+1]⁺; ¹H NMR (400 MHz, DMSO-d₆) δ 8.84 (s, 1H), 8.04 (s, 1H), 7.95 (dd, J=19.0, 8.2 Hz, 2H), 7.71 (s, 1H), 4.13-4.04 (m, 2H), 2.89 (s, 3H).

### Step 3. (R)-N-(1-methylpiperidin-3-yl)-1-(2-(2,2,2-trifluoroethyl)-4-(trifluoromethyl) phenyl)imidazo[1,5-d][1,2,4]triazin-4-amine

To a solution of 4-(methylthio)-1-(2-(2,2,2-trifluoroethyl)-4-(trifluoromethyl)phenyl)-imidazo[1,5-d][1,2,4]triazine (100 mg, 0.25 mmol, 1.0 eq.) in DMA (1 mL) was slowly added (R)-1-methylpiperidin-3-amine dihydrochloride (119 mg, 0.64 mmol, 2.5 eq.) and DIEA (329.4 mg, 10.0 eq.) under N₂ at rt. The reaction mixture was stirred for 12 h at 140° C. under N₂, then filtered and concentrated under reduced pressure. The crude product was purified by reverse phase chromatography (0.1% formic acid in MeCN:0.1% formic acid in H₂O, 5 to 100%) to give title product (16.4 mg, 13.8% yield) as a pale-yellow solid as a formic acid salt. MS m/z 459.0 [M+1]⁺; ¹H NMR (400 MHz, Methanol-d₄) δ 8.77 (s, 1H), 8.49 (s, 1H), 7.92 (s, 1H), 7.85 (dd, J=19.2, 8.2 Hz, 2H), 7.50 (s, 1H), 4.63-4.36 (m, 1H), 3.92 (q, J=10.9 Hz, 2H), 3.64-3.47 (m, 1H), 3.29-3.08 (m, 2H), 3.02-2.79 (m, 2H), 2.72-2.57 (m, 1H), 2.31-1.70 (m, 4H), 1.28 (t, J=6.8 Hz, 3H).

The compounds below were prepared according to the procedure of Example 2 by substituting the appropriate starting materials, reagents and reaction conditions.

| Compound# | Spectral Data |
|---|---|
| 183 | MS m/z 376.3 [ M + 1]⁺. ¹HNMR (400 MHz, Methanol-d₄) δ 8.53-8.79 (m, 1H), |
| | 7.32-7.47 (m, 2H), 7.07-7.19 (m, 2H), 6.46-6.92 (m, 1H), 4.47 (quin, J = 8.2 Hz, |
| | 1H), 3.41-3.52 (m, 2H), 2.46-2.64 (m, 2H), 2.37 (s, 3H), 2.15-2.28 (m, 1H), |
| | 1.77-1.99 (m, 2H). NH and OH not observed. |
| 184 | MS m/z 419.3 [ M + 1]⁺. ¹HNMR (400 MHz, Methanol-d₄) δ 8.64 (s, 1H), 8.33 |
| | (s, 1H), 7.33-7.43 (m, 2H), 7.07-7.21 (m, 2H), 6.37-6.97 (m, 1H), 4.40-4.55 |
| | (m, 1H), 3.69-3.77 (m, 2H), 3.39-3.60 (m, 1H), 3.07-3.17 (m, 1H), 2.85-2.97 |
| | (m, 2H), 2.66-2.87 (m, 2H), 2.37 (s, 3H), 1.89-2.26 (m, 2H), 1.60-1.86 (m, |
| | 2H). NH and OH not observed. |
| 185 | MS m/z 445.4 [ M + 1]⁺. ¹HNMR (400 MHz, Methanol-d₄) δ 8.64 (s, 1H), 8.37 |
| | (s, 1H), 7.28-7.49 (m, 2H), 6.93-7.13 (m, 2H), 6.51-6.92 (m, 1H), 4.21-4.52 |
| | (m, 1H), 3.60-3.79 (m, 2H), 3.29-3.44 (m, 1H), 2.90-3.09 (m, 1H), 2.73-2.89 |
| | (m, 2H), 2.47-2.69 (m, 2H), 1.83-2.13 (m, 3H), 1.58-1.82 (m, 2H), 0.93-1.13 |
| | (m, 2H), 0.60-0.83 (m, 2H). NH and OH not observed. |
| 186 | MS m/z 473.2 [ M + 1]⁺. ¹HNMR (400 MHz, Methanol-d₄) δ 8.77 (s, 1H), 8.38 |
| | (s, 1H), 7.85 (d, J = 8.0 Hz, 1H), 7.75 (d, J = 8.0 Hz, 1H), 7.68 (s, 1H), 7.54 (s, |
| | 1H), 6.88 (d, J = 73.0 Hz, 1H), 4.64-4.49 (m, 1H), 3.94-3.80 (m, 2H), 3.69- |
| | 3.58 (m, 1H), 3.27-3.21 (m, 1H), 3.08 (dd, J = 25.6, 13.8 Hz, 2H), 2.93-2.78 |
| | (m, 2H), 2.20-2.05 (m, 2H), 1.88 (ddd, J = 28.1, 14.1, 8.1 Hz, 2H). NH and |
| | OH not observed. |
| 187 | MS m/z 430.2 [ M + 1]⁺. ¹HNMR (400 MHz, Methanol-d₄) δ 8.82 (s, 1H), 7.86 |
| | (d, J = 8.1 Hz, 1H), 7.75 (d, J = 8.3 Hz, 1H), 7.69 (s, 1H), 7.60 (s, 1H), 6.98 (t, |
| | J = 72.9 Hz, 1H), 4.36-4.18 (m, 1H), 2.67 (td, J = 9.6, 6.0 Hz, 2H), 2.36- |
| | 2.27 (m, 2H), 1.44 (s, 3H). NH and OH not observed |
| 188 | MS m/z 430.5 [ M + 1]⁺. ¹HNMR (400 MHz, Methanol-d₄) δ 8.83 (s, 1H), 7.86 |
| | (d, J = 8.0 Hz, 1H), 7.75 (d, J = 8.0 Hz, 1H), 7.68 (s, 1H), 7.58 (s, 1H), 6.97 (t, |
| | J = 72.9 Hz, 1H), 4.34 (s, 1H), 4.16 (d, J = 10.6 Hz, 1H), 3.90-3.85 (m, 1H), |
| | 3.57-3.46 (m, 2H), 2.24 (s, 1H), 1.84 (dt, J = 21.1, 9.3 Hz, 3H). NH not |
| | observed |
| 189 | MS m/z 396.1, 398.1 [ M + 1]⁺. ¹HNMR (400 MHz, Methanol-d₄) δ 8.91 (s, 1H), |
| | 7.72-7.64 (m, 2H), 7.48 (d, J = 12.3 Hz, 2H), 6.94 (t, J = 72.9 Hz, 1H), 4.34- |
| | 4.26 (m, 1H), 4.18-4.12 (m, 1H), 3.91-3.84 (m, 1H), 3.58-3.48 (m, 2H), |
| | 2.33-2.18 (m, 1H), 1.94-1.83 (m, 2H), 1.83-1.75 (m, 1H). NH not |
| | observed |
| 190 | MS m/z 409.1, 411.1 [ M + 1]⁺. ¹HNMR (400 MHz, Methanol-d₄) δ 8.75 (s, 1H), |
| | 8.45 (br s, 1H), 7.62 (d, J = 8.8 Hz, 1H), 7.51 (br s, 1H), 7.48-7.43 (m, 2H), |
| | 6.91 (t, J = 72.8 Hz, 1H), 4.61-4.36 (m, 1H), 3.59-3.44 (m, 1H), 3.28-3.06 |
| | (m, 2H), 2.94-2.78 (m, 1H), 2.72 (s, 3H), 2.26-2.00 (m, 2H), 1.94-1.69 (m, |
| | 2H). NH not observed |
| 191 | MS m/z 423.1, 425.1 [ M + 1]⁺. ¹HNMR (400 MHz, Methanol-d₄) δ 8.75 (s, 1H), |
| | 8.45 (s, 1H), 7.62 (d, J = 8.7 Hz, 1H), 7.51 (s, 1H), 7.45 (s, 2H), 7.00 (d, J = |
| | 73.2 Hz, 1H), 4.55-4.44 (m, 1H), 3.80-3.64 (m, 1H), 3.42-3.33 (m, 1H), |
| | 3.12 (dd, J = 14.1, 7.1 Hz, 2H), 3.02-2.81 (m, 2H), 2.23-2.10 (m, 2H), 1.96- |
| | 1.83 (m, 2H), 1.34 (t, J = 7.3 Hz, 3H). NH not observed |
| 192 | MS m/z 439.1, 441.1 [ M + 1]⁺. ¹HNMR (400 MHz, Methanol-d₄) δ 8.77 (s, 1H), |
| | 8.43 (s, 1H), 7.62 (d, J = 8.8 Hz, 1H), 7.51 (s, 1H), 7.48-7.42 (m, 2H), 6.91 (t, |
| | J = 73.2 Hz, 1H), 4.62-4.47 (m, 1H), 3.85 (t, J = 5.2 Hz, 2H), 3.66-3.58 (m, |
| | 1H), 3.28-3.21 (m, 1H), 3.10-3.04 (m, 2H), 2.99-2.80 (m, 2H), 2.19-2.07 |
| | (m, 2H), 1.98-1.77 (m, 2H). NH and OH not observed |
| 193 | MS m/z 427.2, 429.2 [ M + 1]⁺. 1H NMR (400 MHz, Methanol-d₄) δ 8.71 (s, |
| | 1H), 8.23 (s, 0.79H), 7.62 (d, J = 8.8 Hz, 1H), 7.52 (s, 1H), 7.48-7.40 (m, 2H), |
| | 6.90 (t, J = 73.2 Hz, 1H), 5.11 (d, J = 46.5 Hz, 1H), 4.80-4.73 (m, 1H), 3.50 |
| | (d, J = 11.6 Hz, 1H), 3.29-3.19 (m, 1H), 2.83-2.71 (m, 1H), 2.60 (s, 3H), |
| | 2.57-2.47 (m, 2H), 2.05-1.90 (m, 1H). NH not observed. |
| 194 | MS m/z 396.0; 398.0 [ M + 1]⁺. ¹HNMR (400 MHz, Methanol-d₄) δ 8.72 (s, 1H), |
| | 7.67-7.59 (m, 1H), 7.47 (d, J = 21.8 Hz, 3H), 6.88 (t, J = 73.3 Hz, 1H), 4.32- |
| | 4.19 (m, 1H), 2.69-2.63 (m, 2H), 2.32-2.23 (m, 2H), 1.44 (s, 3H). NH and |
| | OH not observed |
| 195 | MS m/z 453.1, 455.1 [ M + 1]⁺. ¹HNMR (400 MHz, DMSO-d₆) δ 8.85 (s, 1H), |
| | 7.77 (d, J = 8.4 Hz, 1H), 7.67-7.60 (m, 2H), 7.59-7.54 (m, 1H), 7.47 (s, 1H), |
| | 7.32 (t, J = 73.6, 1H), 4.42-4.20 (m, 1H), 3.18-2.99 (m, 1H), 2.82-2.67 (m, |
| | 1H), 2.25 (s, 3H), 2.10-1.93 (m, 3H), 1.82-1.75 (m, 1H), 1.66-1.55 (m, |
| | 1H), 1.49-1.39 (m, 1H). |
| 196 | MS m/z 485.1, 487.1 [M + H]+; 1H NMR (400 MHz, DMSO-d₆) δ 8.80 (s, 1H), |
| | 7.80 (d, J = 7.4 Hz, 1H), 7.65-7.55 (m, 3H), 7.48 (s, 1H), 7.32 (t, J = 73.6 Hz, |
| | 1H), 5.02 (d, J = 47.1 Hz, 1H), 4.59 (s, 1H), 3.18 (d, J = 12.0 Hz, 1H), 3.02 (t, |
| | J = 12.0 Hz, 1H), 2.48-2.42 (m, 2H), 2.36-2.16 (m, 2H), 2.13-2.00 (m, 1H), |
| | 1.90-1.68 (m, 1H), 1.02 (t, J = 7.1 Hz, 3H). |
| 197 | MS m/z 461.1 [ M + 1]⁺. ¹HNMR (400 MHz, Methanol-d₄) δ 8.79 (s, 1H), 7.96 |
| | (d, J = 8.1 Hz, 1H), 7.90 (d, J = 8.1 Hz, 1H), 7.83 (s, 1H), 7.55 (s, 1H), 4.46 (s, |
| | 1H), 3.21-3.04 (m, 1H), 2.82-2.66 (m, 1H), 2.35 (s, 3H), 2.33-2.17 (m, |
| | 2H), 2.16-2.06 (m, 1H), 1.95-1.83 (m, 1H), 1.82-1.71 (m, 1H), 1.68-1.56 |
| | (m, 1H). NH not observed. |
| 198 | MS m/z 483.2, 485.2 [ M + 1]⁺. ¹HNMR (400 MHz, DMSO-d₆) δ 8.83 (s, 1H), |
| | 7.71 (d, J = 7.5 Hz, 1H), 7.64-7.55 (m, 3H), 7.47 (s, 1H), 7.32 (t, J = 73.6 Hz, |
| | 1H), 4.40 (t, J = 5.2 Hz, 1H), 4.28 (s, 1H), 3.52 (q, J = 5.9 Hz, 2H), 3.16 (d, J = |
| | 6.7 Hz, 1H), 2.82 (d, J = 10.3 Hz, 1H), 2.45 (t, J = 6.2 Hz, 2H), 2.10-1.99 (m, |
| | 3H), 1.80-1.71 (m, 1H), 1.63-1.53 (m, 1H), 1.51-1.36 (m, 1H). |
| 199 | MS m/z 440.1, 442.1 [ M + 1]⁺. ¹HNMR (400 MHz, DMSO-d₆) δ 8.80 (s, 1H), |
| | 8.19 (d, J = 6.0 Hz, 1H), 7.70-7.53 (m, 3H), 7.46 (s, 1H), 7.31 (t, J = 73.6 Hz, |
| | 1H), 5.11 (s, 1H), 4.34-4.12 (m, 1H), 2.49-2.45 (m, 2H), 2.26-2.10 (m, |
| | 2H), 1.33 (s, 3H). |
| 200 | MS m/z 455.1, 457.1 [ M + 1]⁺. ¹HNMR (400 MHz, Methanol-d₄) δ 8.77 (s, 1H), |
| | 8.08 (d, J = 1.6 Hz, 1H), 7.98 (dd, J = 2.0 Hz, J = 8.4 Hz, 1H), 7.57 (d, J = 8.4 |
| | Hz, 1H), 7.37 (s, 1H), 4.45-4.44 (m, 1H), 3.24-3.22 (m, 1H), 2.88-2.82 |
| | (m, 1H), 2.44 (s, 3H), 2.39-2.38 (m, 2H), 2.11-2.09 (m, 1H), 1.95-1.92 (m, |
| | 1H), 1.81-1.76 (m, 1H), 1.67-1.64 (m, 1H). NH not observed. |
| 201 | MS m/z 485.2, 487.2 [ M + 1]⁺. ¹HNMR (400 MHz, Methanol-d₄) δ 8.81 (s, 1H), |
| | 8.08 (d, J = 1.6 Hz, 1H), 7.98 (d, J = 8.0 Hz, 1H), 7.57 (d, J = 8.0 Hz, 1H), 7.38 |
| | (s, 1H), 4.48-4.44 (m, 1H), 3.82-3.69 (m, 2H), 3.28-3.20 (m, 1H), 2.92- |
| | 2.82 (m, 1H), 2.82-2.66 (m, 2H), 2.64-2.47 (m, 2H), 2.07-2.02 (m, 1H), |
| | 1.98-1.89 (m, 1H), 1.83-1.70 (m, 2H). NH and OH not observed. |
| 202 | MS m/z 389.2 [ M + 1]⁺. ¹HNMR (400 MHz, DMSO-d₆) δ 8.82 (s, 1H), 7.67 (d, |
| | J = 7.8 Hz, 1H), 7.49 (d, J = 7.7 Hz, 1H), 7.41 (s, 1H), 7.22 (d, J = 8.1 Hz, 1H), |
| | 7.20 (s, 1H), 7.19 (t, J = 73.6 Hz, 1H), 4.27 (s, 1H), 3.06 (d, J = 10.3 Hz, 1H), |
| | 2.69 (d, J = 12.0 Hz, 1H), 2.43 (s, 3H), 2.22 (s, 3H), 2.06-1.89 (m, 3H), 1.84- |
| | 1.73 (m, 1H), 1.68-1.53 (m, 1H), 1.50-1.34 (m, 1H). |
| 203 | MS m/z 415.3 [ M + 1]⁺. ¹HNMR (400 MHz, DMSO-d₆) δ 8.82 (s, 1H), 7.67 (d, |
| | J = 7.8 Hz, 1H), 7.47 (d, J = 7.9 Hz, 1H), 7.41 (s, 1H), 7.21 (t, J = 73.6 Hz, 1H), |
| | 7.09 (d, J = 8.0 Hz, 1H), 7.06 (s, 1H), 4.28 (s, 1H), 3.11-3.00 (m, 1H), 2.75- |
| | 2.63 (m, 1H), 2.22 (s, 3H), 2.10-1.89 (m, 4H), 1.82-1.72 (m, 1H), 1.65- |
| | 1.53 (m, 1H), 1.49-1.35 (m, 1H), 1.09-1.00 (m, 2H), 0.84-0.76 (m, 2H). |
| 204 | MS m/z 442.2, 444.2 [ M + 1]⁺. ¹HNMR (400 MHz, Methanol-d₄) δ 8.75 (s, 1H), |
| | 8.08 (d, J = 1.6 Hz, 1H), 7.97 (d, J = 1.6 Hz, J = 8.4 Hz, 1H), 7.57 (d, J = 8.4 |
| | Hz, 1H), 7.37 (s, 1H), 4.31-4.23 (m, 1H), 2.68-2.62 (m, 2H), 2.30-2.25 |
| | (m, 2H), 1.44 (s, 3H). NH and OH not observed. |
| 205 | MS m/z 487.1, 489.1 [ M + 1]⁺. 1H NMR (400 MHz, Methanol-d₄) δ 8.65 (s, |
| | 1H), 7.98 (d, J = 1.6 Hz, 1H), 7.88 (d, J = 1.6 Hz, J = 8.0 Hz, 1H), 7.47 (d, J = |
| | 8.4 Hz, 1H), 7.28 (s, 1H), 4.97-4.95 (m, 1H), 4.69-4.63 (m, 1H), 3.03-2.97 |
| | (m, 1H), 2.51-2.46 (m, 2H), 2.37-2.14 (m, 4H), 1.89-1.73 (m, 1H), 1.04 (t, |
| | J = 7.2 Hz, 3H). NH and OH not observed. |
| 206 | MS m/z 391.1 [ M + 1]⁺. ¹HNMR (400 MHz, Methanol-d₄) δ 8.75 (s, 1H), 7.72 |
| | (s, 1H), 7.60 (d, J = 8.0 Hz, 1H), 7.49 (d, J = 7.6 Hz, 1H), 7.30 (s, 1H), 4.62- |
| | 4.37 (m, 1H), 3.20-3.08 (m, 1H), 2.78-2.68 (m, 1H), 2.53 (s, 3H), 2.35 (s, |
| | 3H), 2.32-2.18 (m, 2H), 2.14-2.04 (m, 1H), 1.94-1.84 (m, 1H), 1.82-1.68 |
| | (m, 1H), 1.66-1.55 (m, 1H). NH not observed |
| 207 | MS m/z 417.3 [ M + 1]⁺. ¹HNMR (400 MHz, Methanol-d₄) δ 8.74 (s, 1H), 7.59 |
| | (s, 1H), 7.49-7.43 (m, 2H), 7.30 (s, 1H), 4.48-4.40 (m, 1H), 3.17-3.13 (m, |
| | 1H), 2.78-2.68 (m, 1H), 2.36 (s, 3H), 2.32-2.20 (m, 2H), 2.14-2.06 (m, |
| | 2H), 1.94-1.84 (m, 1H), 1.82-1.70 (m, 1H), 1.70-1.54 (m, 1H), 1.18-1.04 |
| | (m, 2H), 0.88-0.78 (m, 2H). NH not observed |
| 208 | MS m/z 423.1 [ M + 1]⁺. ¹HNMR (400 MHz, Methanol-d₄) δ 8.75 (s, 1H), 7.63 |
| | (d, J = 8.8 Hz, 1H), 7.48 (s, 1H), 7.13 (dd, J = 8.8, 2.4 Hz, 1H), 7.04 (s, 1H), |
| | 4.49-4.36 (m, 1H), 3.92 (s, 3H), 3.19-3.08 (m, 1H), 2.79-2.64 (m, 1H), |
| | 2.34 (s, 3H), 2.32-2.20 (m, 2H), 2.15-2.05 (m, 1H), 1.94-1.82 (m, 1H), |
| | 1.82-1.68 (m, 1H), 1.68-1.51 (m, 1H). NH not observed |
| 209 | MS m/z 437.1 [ M + 1]⁺. ¹HNMR (400 MHz, Methanol-d₄) δ 8.74 (s, 1H), 7.63 |
| | (d, J = 8.8 Hz, 1H), 7.49 (s, 1H), 7.13 (dd, J = 8.8, 2.4 Hz, 1H), 7.05 (s, 1H), |
| | 4.52-4.37 (m, 1H), 3.92 (s, 3H), 3.28-3.18 (m, 1H), 2.88-2.74 (m, 1H), |
| | 2.59-2.48 (m, 2H), 2.34-2.19 (m, 2H), 2.19-2.08 (m, 1H), 1.94-1.83 (m, |
| | 1H), 1.82-1.69 (m, 1H), 1.69-1.55 (m, 1H), 1.15 (t, J = 7.2 Hz, 3H). NH not |
| | observed. |
| 210 | MS m/z 441.2 [ M + 1]⁺. ¹HNMR (400 MHz, Methanol-d₄) δ 8.73 (s, 1H), 7.63 |
| | (d, J = 8.4 Hz, 1H), 7.50 (br s, 1H), 7.14 (dd, J = 8.8, 2.4 Hz, 1H), 7.05 (s, 1H), |
| | 4.71-4.50 (m, 1H), 3.92 (s, 3H), 3.30-3.22 (m, 1H), 3.07-2.96 (m, 1H), |
| | 2.49-2.42 (m, 2H), 2.40-2.30 (m, 1H), 2.36 (s, 3H), 2.27-2.18 (m, 1H), |
| | 1.97-1.77 (m, 1H). NH not observed. |
| 211 | MS m/z 453.1 [ M + 1]⁺. ¹HNMR (400 MHz, Methanol-d₄) δ 8.79 (s, 1H), 7.63 |
| | (d, J = 8.8 Hz, 1H), 7.48 (s, 1H), 7.13 (dd, J = 8.8, 2.4 Hz, 1H), 7.04 (s, 1H), |
| | 4.51-4.40 (m, 1H), 3.92 (s, 3H), 3.77-3.66 (m, 2H), 3.17-3.08 (m, 1H), |
| | 2.75-2.67 (m, 1H), 2.67-2.52 (m, 2H), 2.48-2.36 (m, 2H), 2.10-1.98 (m, |
| | 1H), 1.92-1.81 (m, 1H), 1.81-1.65 (m, 2H). NH and OH not observed. |
| 212 | MS m/z 421.3 [M + H]+; ¹HNMR (400 MHz, Methanol-d₄) δ 8.76 (s, 1H), 7.55 |
| | (d, J = 8.5 Hz, 1H), 7.43-7.29 (m, 3H), 4.63-4.35 (m, 1H), 3.95 (s, 3H), 3.74- |
| | 3.45 (m, 1H), 3.21-3.06 (m, 1H), 3.03-2.81 (m, 2H), 2.77-2.46 (m, 2H), |
| | 2.32-2.13 (m, 1H), 2.10-1.98 (m, 1H), 1.96-1.66 (m, 2H), 1.27 (t, J = 6.9 |
| | Hz, 3H). NH not observed. |
| 213 | MS m/z 437.3 [ M + 1]⁺. ¹HNMR (400 MHz, Methanol-d₄) δ 8.77 (s, 1H), 7.55 |
| | (d, J = 8.5 Hz, 1H), 7.40 (d, J = 2.4 Hz, 1H), 7.37-7.30 (m, 2H), 4.61-4.35 |
| | (m, 1H), 3.95 (s, 3H), 3.86-3.70 (m, 2H), 3.44-3.34 (m, 1H), 3.09-2.90 (m, |
| | 1H), 2.91-2.74 (m, 2H), 2.73-2.45 (m, 2H), 2.21-2.05 (m, 1H), 2.04-1.91 |
| | (m, 1H), 1.90-1.67 (m, 2H). NH and OH not observed. |
| 214 | MS m/z 394.2 [ M + 1]⁺. ¹HNMR (400 MHz, Methanol-d₄) δ 8.80 (s, 1H), 7.55 |
| | (d, J = 8.5 Hz, 1H), 7.49-7.25 (m, 3H), 4.39-4.18 (m, 1H), 3.95 (s, 3H), 2.74- |
| | 2.59 (m, 2H), 2.34-2.19 (m, 2H), 1.44 (s, 3H). NH and OH not observed. |
| 215 | MS m/z 446.3 [ M + 1]⁺. ¹HNMR (400 MHz, Methanol-d₄) δ 8.81 (s, 1H), 8.20 |
| | (s, 1H), 8.14 (d, J = 8.0 Hz, 1H), 7.90 (d, J = 8.0 Hz, 1H), 7.43 (s, 1H), 4.46- |
| | 4.08 (m, 1H), 3.82-3.50 (m, 1H), 2.67-2.35 (m, 1H), 2.32-2.12 (m, 1H), |
| | 2.08-1.93 (m, 1H), 1.93-1.74 (m, 1H), 1.52-1.12 (m, 4H). NH and OH not |
| | observed. |
| 216 | MS m/z 405.13 [ M + 1]⁺. ¹HNMR (Methanol-d₄) δ: 8.74 (s, 1H), 8.35-8.59 (m, |
| | 1H), 7.45-7.56 (m, 2H), 7.00-7.04 (m, 1H), 6.90-6.98 (m, 1H), 6.87 (s, 1H), |
| | 4.35-4.61 (m, 1H), 3.92 (s, 3H), 3.49-3.66 (m, 1H), 3.03-3.18 (m, 2H), 2.67- |
| | 2.81 (m, 4H), 2.02-2.24 (m, 2H), 1.71-1.95 (m, 2H). NH not observed. |
| 217 | MS m/z 445.3 [ M + 1]⁺. ¹HNMR (400 MHz, Methanol-d₄) δ 8.70 (s, 1H), 8.10 |
| | (s, 1H), 8.04 (d, J = 8.1 Hz, 1H), 7.79 (d, J = 8.1 Hz, 1H), 7.31 (s, 1H), 4.44- |
| | 4.29 (m, 1H), 3.18-3.10 (m, 1H), 2.89-2.69 (m, 1H), 2.51-2.24 (m, 5H), |
| | 2.09-1.95 (m, 1H), 1.94-1.78 (m, 1H), 1.77-1.62 (m, 1H), 1.61-1.49 (m, |
| | 1H). NH not observed. |
| 218 | MS m/z 432.3 [ M + 1]⁺. ¹HNMR (400 MHz, Methanol-d₄) δ 9.02 (s, 1H), 8.20 |
| | (s, 1H), 8.14 (d, J = 7.9 Hz, 1H), 7.89 (d, J = 8.0 Hz, 1H), 7.68 (s, 1H), 4.27 |
| | (quint., J = 8.0 Hz, 1H), 2.75-2.57 (m, 2H), 2.42-2.20 (m, 2H), 1.44 (s, 3H). |
| | NH and OH not observed. |
| 219 | MS m/z 410.0 [ M + 1]⁺. ¹HNMR (400 MHz, Methanol-d₄) 8.74 (s, 1H), 7.63 (d, |
| | J = 8.8 Hz, 1H), 7.49 (s, 1H), 7.13 (dd, J = 8.8, 2.4 Hz, 1H), 7.04 (s, 1H), 4.34- |
| | 4.20 (m, 1H), 3.92 (s, 3H), 2.71-2.62 (m, 2H), 2.32-2.23 (m, 2H), 1.44 (s, |
| | 3H). NH and OH not observed |
| 220 | MS m/z 392.0 [ M + 1]⁺. ¹HNMR (400 MHz, Methanol-d₄) δ 8.79 (s, 1H), 7.66 |
| | (d, J = 8.8 Hz, 1H), 7.62 (d, J = 2.4 Hz, 1H), 7.41 (s, 1H), 7.12 (dd, J = 8.8, 2.4 |
| | Hz, 1H), 7.04 (br s, 1H), 6.23 (d, J = 2.0 Hz, 1H), 3.92 (s, 3H). NH not |
| | observed. |
| 221 | MS m/z 410.0 [ M + 1]⁺. ¹HNMR (400 MHz, Methanol-d₄) δ 8.78 (s, 1H), 7.64 |
| | (d, J = 8.4 Hz, 1H), 7.50 (s, 1H), 7.13 (dd, J = 8.4, 2.4 Hz, 1H), 7.05 (s, 1H), |
| | 4.39-4.29 (m, 1H), 4.29-4.22 (m, 1H), 3.92 (s, 3H), 2.45-2.30 (m, 1H), |
| | 2.15-1.99 (m, 1H), 1.96-1.66 (m, 4H). NH and OH not observed |
| 222 | MS m/z 424.1 [ M + 1]⁺. ¹HNMR (400 MHz, Methanol-d₄) δ 8.73 (s, 1H), 7.64 |
| | (d, J = 8.4 Hz, 1H), 7.49 (s, 1H), 7.13 (dd, J = 8.8, 2.4 Hz, 1H), 7.05 (s, 1H), |
| | 4.31-4.16 (m, 1H), 3.92 (s, 3H), 3.78-3.67 (m, 1H), 2.53-2.42 (m, 1H), |
| | 2.23-2.12 (m, 1H), 2.05-1.95 (m, 1H), 1.92-1.84 (m, 1H), 1.47-1.38 (m, |
| | 2H), 1.33-1.20 (m, 2H). NH and OH not observed |
| 223 | MS m/z 424.1 [ M + 1]⁺. ¹HNMR (400 MHz, Methanol-d₄) δ 8.76 (s, 1H), 7.63 |
| | (d, J = 8.8 Hz, 1H), 7.47 (s, 1H), 7.13 (dd, J = 8.4, 2.0 Hz, 1H), 7.05 (s, 1H), |
| | 4.26-4.06 (m, 1H), 3.92 (s, 3H), 3.71-3.58 (m, 1H), 2.30-2.18 (m, 1H), |
| | 2.17-2.05 (m, 1H), 1.89-1.75 (m, 2H), 1.53-1.35 (m, 4H). NH and OH not |
| | observed |
| 224 | MS m/z 423.1 [ M + 1]⁺. ¹HNMR (500 MHz, Methanol-d₄) δ 8.57-8.93 (m, 1H), |
| | 7.50-7.63 (m, 2H), 6.96-7.07 (m, 2H), 6.82-6.96 (m, 2H), 5.02-5.26 (m, 1H), |
| | 4.72-4.85 (m, 1H), 3.92 (s, 3H), 3.45-3.63 (m, 1H), 2.72-2.85 (m, 1H), 2.68 (s, |
| | 1H), 2.61 (s, 3H), 2.40-2.56 (m, 2H), 1.86-2.06 (m, 1H). NH not observed. |
| 225 | MS m/z 435.15 [ M + 1]⁺. ¹HNMR (500 MHz, Methanol-d₄) δ 8.76 (s, 1H), 7.49- |
| | 7.66 (m, 2H), 6.97-7.10 (m, 2H), 6.84-6.96 (m, 2H), 4.45-4.73 (m, 1H), 3.92 (s, |
| | 3H), 3.80-3.92 (m, 2H), 3.57-3.74 (m, 1H), 3.20-3.30 (m, 2H), 3.00-3.11 (m, |
| | 2H), 2.81-2.98 (m, 2H), 2.01-2.29 (m, 2H), 1.75-2.00 (m, 2H). NH and OH not |
| | observed. |
| 226 | MS m/z 392.15 [ M + 1]⁺. ¹HNMR (500 MHz, Methanol-d₄) δ 8.72 (s, 1H), 7.48- |
| | 7.59 (m, 1H), 7.50 (s, 1H), 6.96-7.03 (m, 1H), 6.92 (s, 1H), 6.85 (s, 1H), 4.20- |
| | 4.41 (m, 1H), 3.91 (s, 3H), 2.60-2.75 (m, 2H), 2.19-2.38 (m, 2H), 1.46 (s, 3H). |
| | NH and OH not observed. |
| 227 | MS m/z 374.05 [M + H]⁺. ¹HNMR (500 MHz, Methanol-d₄) δ 8.72-8.93 (m, |
| | 1H), 7.55-7.73 (m, 2H), 7.35-7.50 (m, 1H), 6.97-7.13 (m, 1H), 6.83-6.94 (m, |
| | 2H), 6.24 (s, 1H), 5.51 (s, 1H), 3.92 (s, 3H). NH not observed. |
| 228 | MS m/z 376.1 [M + H]⁺. ¹HNMR (400 MHz, Methanol-d₄) δ 8.68 (s, 1H), 8.44 |
| | (s, 1H, Formate), 7.56-7.47 (m, 2H), 7.30-7.26 (m, 1H), 7.25-7.19 (m, |
| | 1H), 7.17-7.11 (m, 1H), 6.16-6.08 (m, 1H), 3.84 (s, 3H). NH not observed. |
| 229 | MS m/z 475.1 [M + H]⁺. ¹HNMR (400 MHz, Methanol-d₄) δ 8.78 (s, 1H), 7.93 |
| | (dd, J = 22.1, 8.5 Hz, 2H), 7.83 (s, 1H), 7.55 (s, 1H), 4.47 (s, 1H), 3.27-3.17 |
| | (m, 1H), 2.83 (s, 1H), 2.58-2.50 (m, 2H), 2.26 (s, 2H), 2.14 (s, 1H), 1.87 (s, |
| | 1H), 1.77 (d, J = 10.0 Hz, 1H), 1.64 (s, 1H), 1.15 (t, J = 7.2 Hz, 3H). NH not |
| | observed. |
| 230 | MS m/z 460.1 [M + H]⁺. ¹HNMR (400 MHz, Methanol-d₄) δ 8.85 (s, 1H), 7.60 |
| | (d, J = 8.4 Hz, 1H), 7.49 (s, 1H), 7.25 (t, J = 8.4 Hz, 1H), 7.11-7.06 (m, 3H), |
| | 7.02 (s, 1H), 6.81 (dd, J = 2.0 Hz, J = 9.2 Hz, 1H), 5.52-5.47 (m, 1H), 3.90 (s, |
| | 3H), 3.78 (s, 3H), 1.70 (d, J = 7.2 Hz, 3H). NH not observed. |
| 231 | MS m/z 430.1 [M + H]⁺. ¹HNMR (400 MHz, Methanol-d₄) δ 8.85 (s, 1H), 7.59 |
| | (d, J = 8.4 Hz, 1H), 7.50 (d, J = 7.6 Hz, 3H), 7.34 (t, J = 7.6 Hz, 2H), 7.24 (t, |
| | J = 7.2 Hz, 1H), 7.10 (dd, J = 2.4 Hz, J = 8.8 Hz, 1H), 7.02 (s, 1H), 5.57-5.49 |
| | (m, 1H), 3.90 (s, 3H), 1.71 (d, J = 6.8 Hz, 3H). NH not observed. |
| 232 | MS m/z 431.1 [M + H]⁺. ¹HNMR (400 MHz, Methanol-d₄) δ 8.83 (s, 1H), 8.71 |
| | (d, J = 1.6 Hz, 1H), 8.44 (dd, J = 1.2 Hz, J = 4.8 Hz, 1H), 8.00 (d, J = 8.0 Hz, |
| | 1H), 7.59 (d, J = 8.8 Hz, 1H), 7.50 (s, 1H), 7.45-7.42 (m, 1H), 7.10 (dd, J = |
| | 2.4 Hz, J = 8.4 Hz, 1H), 7.03 (s, 1H), 5.56-5.54 (m, 1H), 3.90 (s, 3H), 1.76 (d, |
| | J = 6.8 Hz, 3H). NH not observed. |
| 233 | MS m/z 432.0 [M + H]⁺. ¹HNMR (400 MHz, Methanol-d₄) δ 8.70 (s, 1H), 7.64 |
| | (d, J = 8.8 Hz, 1H), 7.50 (s, 1H), 7.19-7.11 (m, 2H), 7.04 (s, 1H), 6.95-6.92 |
| | (m, 2H), 6.74 (dd, J = 2.0 Hz, J = 8.0 Hz, 1H), 4.80 (s, 2H), 3.91 (s, 3H). NH |
| | and OH not observed. |
| 234 | MS m/z 406.0 [M + H]⁺. ¹HNMR (400 MHz, Methanol-d₄) δ 8.69 (s, 1H), 7.65 |
| | (d, J = 8.8 Hz, 1H), 7.61 (s, 1H), 7.50 (s, 1H), 7.13 (dd, J = 2.4 Hz, J = 8.8 Hz, |
| | 1H), 7.05 (s, 1H), 6.43 (s, 1H), 4.91 (s, 2H), 3.92 (s, 3H). NH not observed. |
| 235 | MS m/z 393.2 [M + H]⁺. ¹HNMR (400 MHz, Methanol-d4) δ 8.68 (s, 1H), 7.56 |
| | (d, J = 8.5 Hz, 1H), 7.43-7.28 (m, 3H), 5.13-5.00 (m, 1H), 3.95 (s, 3H), 3.57 |
| | -3.42 (m, 2H), 2.86-2.67 (m, 1H), 2.44-2.24 (m, 1H). NH not observed. |
| 236 | MS m/z 408.2 [M + H]⁺. ¹HNMR (400 MHz, Methanol-d₄) δ 8.86 (s, 1H), 7.60 |
| | (s, 1H), 7.56 (d, J = 8.5 Hz, 1H), 7.16 (s, 1H), 7.13 (dd, J = 8.5, 2.4 Hz, 1H), |
| | 4.28-4.18 (m, 1H), 3.91 (s, 3H), 3.77 (q, J = 10.9 Hz, 2H), 2.74-2.62 (m, |
| | 2H), 2.39-2.26 (m, 2H), 1.45 (s, 3H). 2H not observed (NH and OH). |
| 237 | MS m/z 422.2 [M + H]+; ¹HNMR (400 MHz, Methanol-d₄) δ 8.78 (s, 1H), 7.54 |
| | (d, J = 8.4 Hz, 1H), 7.46 (s, 1H), 7.15-7.07 (m, 2H), 4.17-4.04 (m, 1H), 3.90 |
| | (s, 3H), 3.87-3.69 (m, 2H), 3.69-3.60 (m, 1H), 2.30-2.19 (m, 1H), 2.17- |
| | 2.08 (m, 1H), 1.88-1.76 (m, 2H), 1.53-1.34 (m, 4H). 2H not observed (NH |
| | and OH). |
| 238 | MS m/z 407.2 [M + H]⁺. ¹HNMR (400 MHz, Methanol-d₄) δ 8.68 (s, 1H), 7.54 |
| | (d, J = 8.4 Hz, 1H), 7.48 (s, 1H), 7.16-7.08 (m, 2H), 5.11-5.02 (m, 1H), 3.90 |
| | (s, 3H), 3.77 (q, J = 11.0 Hz, 2H), 3.53-3.44 (m, 2H), 2.80-2.69 (m, 1H), |
| | 2.42-2.26 (m, 1H). 2H not observed (2NH). |
| 239 | MS m/z 491.1 [M + H]⁺. ¹HNMR (400 MHz, Methanol-d₄) δ 8.83 (s, 1H), 7.96 |
| | (d, J = 8.1 Hz, 1H), 7.90 (d, J = 8.2 Hz, 1H), 7.83 (s, 1H), 7.55 (s, 1H), 4.56- |
| | 4.42 (m, 1H), 3.76-3.68 (m, 2H), 3.16-3.07 (m, 1H), 2.73-2.66 (m, 1H), |
| | 2.64-2.54 (m, 2H), 2.50-2.40 (m, 2H), 2.08-2.00 (m, 1H), 1.92-1.83 (m, |
| | 1H), 1.80-1.68 (m, 2H). NH and OH not observed. |
| 240 | MS m/z 462.1 [M + H]⁺. ¹HNMR (400 MHz, DMSO-d₆) δ 8.85 (s, 1H), 7.99 (s, |
| | 2H), 7.96 (s, 1H), 7.52 (s, 1H), 4.75 (s, 1H), 4.18 (s, 1H), 3.55 (t, J = 10.7 Hz, |
| | 1H), 2.31 (d, J = 11.4 Hz, 1H), 2.06 (d, J = 12.4 Hz, 1H), 1.88 (d, J = 11.6 Hz, |
| | 1H), 1.78 (d, J = 13.3 Hz, 1H), 1.34 (dd, J = 23.3, 11.8 Hz, 3H), 1.18-1.09 |
| | (m, 1H). |
| 241 | MS m/z 448.1 [M + H]⁺. ¹HNMR (400 MHz, DMSO-d₆) δ 8.86 (s, 1H), 8.32 (d, |
| | J = 6.1 Hz, 1H), 7.99 (s, 2H), 7.96 (s, 1H), 7.53 (s, 1H), 5.12 (s, 1H), 4.25 (dd, |
| | J = 14.3, 7.8 Hz, 1H), 2.53 (s, 1H), 2.48 (s, 1H), 2.22 (dd, J = 11.3, 8.9 Hz, |
| | 2H), 1.34 (s, 3H). |
| 242 | MS m/z 462.1 [M + H]⁺. 1H NMR (400 MHz, DMSO-d₆) δ 8.91 (s, 1H), 7.99 (s, |
| | 2H), 7.96 (s, 2H), 7.52 (s, 1H), 4.89 (d, J = 4.8 Hz, 1H), 4.09 (s, 1H), 3.57 (s, |
| | 1H), 2.09 (s, 1H), 1.99 (s, 1H), 1.72 (s, 2H), 1.33 (d, J = 5.4 Hz, 4H). |
| 243 | MS m/z 446.1 [M + H]+; 1H NMR (400 MHz, Methanol-d₄) δ 8.81 (s, 1H), 8.20 |
| | (s, 1H), 8.14 (d, J = 7.5 Hz, 1H), 7.89 (d, J = 7.7 Hz, 1H), 7.38 (s, 1H), 4.15 (s, |
| | 1H), 3.73-3.56 (m, 1H), 2.28-2.17 (m, 1H), 2.14-2.06 (m, 1H), 1.94-1.69 |
| | (m, 2H), 1.51-1.39 (m, 4H). NH and OH not observed. |
| 244 | MS m/z 446.1 [M + H]⁺. ¹HNMR (400 MHz, DMSO-d₆) δ 8.83 (s, 1H), 8.23 (d, |
| | J = 6.1 Hz, 1H), 7.80 (d, J = 8.9 Hz, 1H), 7.46-7.40 (m, 3H), 7.28 (t, J = 72.0 |
| | Hz, 1H), 5.11 (s, 1H), 4.23 (dd, J = 13.9, 7.5 Hz, 1H), 2.50-2.46 (m, 2H), |
| | 2.21 (dd, J = 11.3, 8.8 Hz, 2H), 1.31 (d, J = 25.2 Hz, 3H). |
| 245 | MS m/z 460.1 [M + H]⁺. ¹HNMR (400 MHz, DMSO-d₆) δ 8.85 (s, 1H), 7.89 (d, |
| | J = 7.5 Hz, 1H), 7.80 (d, J = 8.9 Hz, 1H), 7.46-7.40 (m, 3H), 7.28 (t, J = 73.2 |
| | Hz, 1H), 4.75 (d, J = 4.6 Hz, 1H), 4.28-4.05 (m, 1H), 3.54 (td, J = 6.5, 2.3 Hz, |
| | 1H), 2.31 (d, J = 11.6 Hz, 1H), 2.13-2.00 (m, 1H), 1.88 (d, J = 10.4 Hz, 1H), |
| | 1.77 (dd, J = 9.4, 3.7 Hz, 1H), 1.44-1.22 (m, 3H), 1.13 (d, J = 14.3 Hz, 1H). |
| 246 | MS m/z 460.0 [M + H]⁺. ¹HNMR (400 MHz, DMSO-d₆) δ 8.85 (s, 1H), 7.80 (d, |
| | J = 8.9 Hz, 1H), 7.45-7.39 (m, 3H), 7.28 (t, J = 73.2 Hz, 1H), 6.0 (bs, 1H) |
| | 4.98-4.78 (m, 1H), 4.11-4.00 (m, 1H), 3.61-3.48 (m, 1H), 2.14-2.06 (m, |
| | 1H), 2.03-1.96 (m, 1H), 1.76-1.68 (m, 2H), 1.37-1.27 (m, 4H). |
| 247 | MS m/z 446.1 [M + H]⁺. ¹HNMR (400 MHz, Methanol-d₄) δ 8.86 (s, 1H), 7.60 |
| | (d, J = 8.4 Hz, 1H), 7.48 (s, 1H), 7.14 (t, J = 7.6 Hz, 1H), 7.10 (dd, J = 2.0 Hz, |
| | J = 8.4 Hz, 1H), 7.03 (s, 1H), 6.94-6.92 (m, 2H), 6.66 (dd, J = 2.4 Hz, J = 7.2 |
| | Hz, 1H), 5.48-5.43 (m, 1H), 3.91 (s, 3H), 1.69 (d, J = 6.8 Hz, 3H). NH and |
| | OH not observed. |
| 248 | MS m/z 446.1 [M + H]⁺. ¹HNMR (400 MHz, Methanol-d₄) δ 8.86 (s, 1H), 7.60 |
| | (d, J = 8.4 Hz, 1H), 7.48 (s, 1H), 7.13 (t, J = 8.4 Hz, 1H), 7.10 (dd, J = 2.0 Hz, |
| | J = 8.4 Hz, 1H), 7.02 (s, 1H), 6.93-6.91 (m, 2H), 6.66 (dd, J = 2.0 Hz, J = 7.2 |
| | Hz, 1H), 5.48-5.43 (m, 1H), 3.90 (s, 3H), 1.69 (d, J = 7.2 Hz, 3H). NH and |
| | OH not observed. |
| 249 | MS m/z 424.3 [M + H]⁺. ¹HNMR (400 MHz, Methanol-d₄) δ 9.06-8.72 (m, |
| | 1H), 7.77-7.45 (m, 2H), 7.15-7.08 (m, 2H), 4.37-4.21 (m, 1H), 4.04 (dd, |
| | J = 11.1, 4.8 Hz, 1H), 3.98 (dd, J = 11.6, 4.1 Hz, 1H), 3.90 (s, 3H), 3.86-3.71 |
| | (m, 3H), 3.56 (t, J = 11.3 Hz, 1H), 3.28 (t, J = 10.6 Hz, 1H), 2.26 (d, J = 12.1 |
| | Hz, 1H), 1.77 (qd, J = 12.4, 4.4 Hz, 1H). 2H not observed (NH and OH). |
| 250 | MS m/z 421.4 [M + H]⁺. ¹HNMR (400 MHz, Methanol-d₄) δ 8.74 (s, 1H), 8.48 |
| | (s, 1H, formic acid), 7.53 (d, J = 8.5 Hz, 1H), 7.45 (br s, 1H), 7.15-7.08 (m, |
| | 2H), 4.54-4.35 (m, 1H), 3.90 (s, 3H), 3.78 (q, J = 11.0, 2H), 3.51-3.38 (m, |
| | 1H), 3.14-2.98 (m, 1H), 2.76-2.54 (m, 5H), 2.28-1.97 (m, 2H), 1.92-1.62 |
| | (m, 2H). 1H not observed (NH). |
| 251 | MS m/z 430.2 [M + H]⁺. ¹HNMR (400 MHz, Methanol-d4) δ 8.88 (s, 1H), 7.53 |
| | (d, J = 8.5 Hz, 1H), 7.42-7.24 (m, 3H), 7.16 (t, J = 7.9 Hz, 1H), 7.01-6.90 |
| | (m, 2H), 6.68 (d, J = 8.1 Hz, 1H), 5.47 (q, J = 7.2 Hz, 1H), 3.94 (s, 3H), 1.70 |
| | (d, J = 6.9 Hz, 3H). NH and OH not observed. |
| 252 | MS m/z 463.3 [M + H]⁺. ¹HNMR (400 MHz, Methanol-d₄) δ 8.82 (s, 1H), 8.22 |
| | (s, 1H), 8.16 (d, J = 8.0 Hz, 1H), 7.91 (d, J = 8.0 Hz, 1H), 7.47 (s, 1H), 5.58- |
| | 5.10 (m, 1H), 4.89-4.69 (m, 1H), 3.81-3.56 (m, 1H), 3.55-3.36 (m, 1H), |
| | 3.02-2.81 (m, 1H), 2.77-2.66 (m, 4H), 2.63-2.40 (m, 1H), 2.25-1.85 (m, |
| | 1H). 1H not observed (NH). |
| 253 | MS m/z 475.4 [M + H]⁺. ¹HNMR (400 MHz, Methanol-d₄) δ 8.88 (s, 1H), 8.22 |
| | (s, 1H), 8.16 (d, J = 8.1 Hz, 1H), 7.92 (d, J = 8.0 Hz, 1H), 7.42 (s, 1H), 4.60- |
| | 4.42 (m, 1H), 3.99-3.69 (m, 2H), 3.25-3.08 (m, 1H), 2.96-2.61 (m, 3H), |
| | 2.58-2.39 (m, 2H), 2.23-2.02 (m, 1H), 2.01-1.84 (m, 1H), 1.84-1.68 (m, |
| | 2H). NH and OH not observed. |
| 254 | MS m/z 432.3 [M + H]⁺. ¹HNMR (400 MHz, Methanol-d₄) δ 8.95 (s, 1H), 8.22 |
| | (s, 1H), 8.16 (d, J = 8.0 Hz, 1H), 7.91 (d, J = 8.0 Hz, 1H), 7.56 (s, 1H), 4.42- |
| | 4.34 (m, 1H), 4.27-4.12 (m, 1H), 4.01-3.81 (m, 1H), 3.71-3.42 (m, 2H), |
| | 2.38-2.18 (m, 1H), 2.06-1.68 (m, 3H). 1H not observed (NH). |
| 255 | MS m/z 448.1 [M + H]⁺. ¹HNMR (400 MHz, DMSO-d₆) δ 8.86 (s, 1H), 8.00 (s, |
| | 2H), 7.97 (s, 1H), 7.52 (s, 1H), 4.60-4.52 (m, 1H), 3.44 (d, J = 5.6 Hz, 2H), |
| | 2.49-2.39 (m, 2H), 2.25-2.16 (m, 1H), 2.00-1.88 (m, 2H). NH and OH not |
| | observed. |
| 256 | MS m/z 447.1 [M + H]+; ¹HNMR (400 MHz, Methanol-d₄) δ 8.73 (s, 1H), 7.96 |
| | (d, J = 8.0 Hz, 1H), 7.91 (d, J = 8.1 Hz, 1H), 7.84 (s, 1H), 7.59 (s, 1H), 5.10 (t, |
| | J = 9.6 Hz, 1H), 3.55-3.47 (m, 2H), 2.81-2.69 (m, 1H), 2.43-2.28 (m, 1H). |
| | NH not observed. |
| 257 | MS m/z 432.0 [M + H]+; ¹HNMR (400 MHz, Methanol-d₄) δ 8.94 (s, 1H), 8.24 |
| | (s, 1H), 8.17 (d, J = 8.4 Hz, 1H), 7.93 (d, J = 8.0 Hz, 1H), 7.54 (s, 1H), 4.55- |
| | 4.46 (m, 1H), 3.60 (d, J = 6.0 Hz, 2H), 2.69-2.62 (m, 2H), 2.38-2.34 (m, 1H), |
| | 2.12-2.04 (m, 2H). NH and OH not observed. |
| 258 | MS m/z 489.1 [M + H]⁺. ¹HNMR (400 MHz, DMSO-d₆) δ 8.85 (s, 1H), 7.81 (d, |
| | J = 8.4 Hz, 1H), 7.75 (s, 1H), 7.46 (t, J = 73.2 Hz, 1H), 7.43 (s, 1H), 7.41 (d, |
| | J = 2.4, 1H), 4.42-4.40 (m, 1H), 4.30-4.29 (m, 1H), 3.53 (q, J = 5.6, 2H), 3.17 |
| | (d, J = 9.6 Hz, 1H), 2.82 (d, J = 10.8 Hz, 1H), 2.47-2.44 (m, 1H), 2.11-2.00 (m, |
| | 3H), 1.77-1.74 (m, 1H), 1.63-1.54 (m, 1H), 1.48-1.43 (m, 1H). NH and OH not |
| | observed. |
| 259 | MS m/z 482.1 [M + H]⁺. ¹HNMR (400 MHz, DMSO-d₆) δ 8.98 (s, 1H), 7.79 (d, |
| | J = 8.0 Hz, 1H), 7.47 (s, 1H), 7.45 (t, J = 73.2 Hz, 1H), 7.41-7.38 (m, 2H), |
| | 7.16-7.12 (m, 1H), 6.92-6.88 (m, 2H), 6.65-6.63 (m, 1H), 5.45-5.43 (m, |
| | 1H), 1.61 (d, J = 6.8 Hz, 3H). NH and OH not observed. |
| 260 | MS m/z 445.0 [M + H]⁺. ¹HNMR (400 MHz, Methanol-d₄) δ 8.70 (s, 1H), 7.77 |
| | (d, J = 8.4 Hz, 1H), 7.55 (s, 1H), 7.37 (d, J = 8.4 Hz, 1H), 7.33 (s, 1H), 7.05 (t, |
| | J = 72.8 Hz, 1H), 5.10-5.05 (m, 1H), 3.52-3.47 (m, 2H), 2.75-2.73 (m, |
| | 1H), 2.41-2.31 (m, 1H). NH not observed. |
| 261 | MS m/z 473.1 [M + H]⁺. ¹HNMR (400 MHz, Methanol-d₄) δ 8.77 (s, 1H), 8.49 |
| | (s, 1H), 7.92 (s, 1H), 7.85 (dd, J = 19.2, 8.2 Hz, 2H), 7.50 (s, 1H), 4.63-4.36 |
| | (m, 1H), 3.92 (q, J = 10.9 Hz, 2H), 3.64-3.47 (m, 1H), 3.29-3.08 (m, 2H), |
| | 3.02-2.79 (m, 2H), 2.72-2.57 (m, 1H), 2.31-1.70 (m, 4H), 1.28 (t, J = 6.8 |
| | Hz, 3H). NH not observed. |
| 262 | MS m/z 460.0 [M + H]⁺. ¹HNMR (400 MHz, Methanol-d₄) δ 8.77 (s, 1H), 7.92 |
| | (s, 1H), 7.85 (d, J = 4.8 Hz, 2H), 7.49 (s, 1H), 4.33-4.14 (m, 1H), 3.94 (q, J = |
| | 10.9 Hz, 2H), 3.85-3.63 (m, 1H), 2.47 (d, J = 11.4 Hz, 1H), 2.17 (d, J = 11.8 |
| | Hz, 1H), 2.00 (d, J = 12.4 Hz, 1H), 1.90 (d, J = 13.4 Hz, 1H), 1.55-1.19 (m, |
| | 4H). NH and OH not observed. |
| 263 | MS m/z 446.0 [M + H]⁺. ¹HNMR (400 MHz, Methanol-d₄) δ 8.80 (s, 1H), 7.92 |
| | (s, 1H), 7.85 (q, J = 8.1 Hz, 2H), 7.51 (s, 1H), 4.33-4.17 (m, 1H), 3.93 (q, J = |
| | 11.0 Hz, 2H), 2.71-2.60 (m, 2H), 2.30 (t, J = 10.2 Hz, 2H), 1.45 (s, 3H). NH |
| | and OH not observed. |
| 264 | MS m/z 446.0 [M + H]⁺. ¹HNMR (400 MHz, Methanol-d₄) δ 8.77 (s, 1H), 7.91 |
| | (s, 1H), 7.84 (q, J = 8.3 Hz, 2H), 7.48 (s, 1H), 4.66-4.54 (m, 1H), 3.94 (q, J = |
| | 11.0 Hz, 2H), 3.59 (d, J = 6.0 Hz, 2H), 2.64 (dt, J = 10.0, 3.7 Hz, 2H), 2.34 (d, |
| | J = 7.7 Hz, 1H), 1.98 (dd, J = 20.8, 9.3 Hz, 2H). NH and OH not observed. |
| 265 | MS m/z 445.0 [M + H]⁺. ¹HNMR (400 MHz, Methanol-d₄) δ 8.72 (s, 1H), 7.97- |
| | 7.72 (m, 3H), 7.51 (s, 1H), 5.09 (t, J = 9.5 Hz, 1H), 3.91 (q, J = 11.0 Hz, 2H), |
| | 3.58-3.42 (m, 2H), 2.75 (s, 1H), 2.37 (dd, J = 20.9, 10.7 Hz, 1H). NH not |
| | observed. |
| 266 | MS m/z 482.0 [M + H]⁺. ¹HNMR (400 MHz, Methanol-d₄) δ 8.99 (s, 1H), 7.93 |
| | (s, 1H), 7.84 (dd, J = 20.5, 8.1 Hz, 2H), 7.58 (s, 1H), 7.18 (t, J = 7.9 Hz, 1H), |
| | 7.01-6.91 (m, 2H), 6.71 (dd, J = 7.9, 2.1 Hz, 1H), 5.43 (q, J = 6.9 Hz, 1H), |
| | 3.88 (tdd, J = 15.1, 10.9, 4.3 Hz, 2H), 1.73 (d, J = 7.0 Hz, 3H). NH and OH not |
| | observed. |
| 267 | MS m/z 428.0 [M + H]⁺. ¹HNMR (400 MHz, Methanol-d₄) δ 8.82 (s, 1H), 7.89 |
| | (d, J = 15.4 Hz, 3H), 7.63 (d, J = 2.3 Hz, 1H), 7.40 (s, 1H), 6.24 (d, J = 2.4 Hz, |
| | 1H), 3.96 (q, J = 10.8 Hz, 2H). NH not observed. |
| 268 | MS m/z 408.4 [M + H]⁺. ¹HNMR (400 MHz, Methanol-d₄) δ 8.64 (s, 1H), 7.32- |
| | 7.51 (m, 2H), 6.90 (s, 1H), 6.67-6.85 (m, 2H), 4.03-4.35 (m, 1H), 3.86-3.99 (m, |
| | 2H), 3.81 (s, 3H), 3.59-3.72 (m, 1H), 3.31-3.58 (m, 1H), 3.10-3.19 (m, 1H), |
| | 2.08-2.31 (m, 1H), 1.51-1.77 (m, 1H). NH and OH not observed. |
| 269 | MS m/z 391.2 [M + H]⁺. ¹HNMR (400 MHz, Methanol-d₄) δ 8.56 (s, 1H), 7.29- |
| | 7.55 (m, 2H), 6.86-6.96 (m, 1H), 6.69-6.85 (m, 2H), 4.86-5.05 (m, 1H), 3.81 (s, |
| | 3H), 3.31-3.48 (m, 2H), 2.64 (s, 1H), 2.02-2.25 (m, 1H). NH not observed. |
| 270 | MS m/z 406.3 [M + H]⁺. ¹HNMR (400 MHz, Methanol-d₄) δ 8.63 (s, 1H), 7.39- |
| | 7.51 (3, 1H), 7.37 (s, 1H), 6.86-6.96 (m, 1H), 6.67-6.84 (m, 2H), 3.95-4.17 (m, |
| | 1H), 3.80 (s, 3H), 3.49-3.62 (m, 1H), 2.12-2.18 (m, 1H), 1.97-2.11 (m, 1H), |
| | 1.57-1.82 (m, 2H), 1.23-1.47 (m, 4H). NH and OH not observed. |
| 271 | MS m/z 459.3 [M + H]⁺. ¹HNMR (400 MHz, Methanol-d₄) δ 8.81 (s, 1H), 8.22 |
| | (s, 1H), 8.16 (d, J = 8.1 Hz, 1H), 7.91 (d, J = 8.0 Hz, 1H), 7.42 (s, 1H), 4.54- |
| | 4.38 (m, 1H), 2.94-2.83 (m, 1H), 2.83-2.61 (m, 2H), 2.52-2.31 (m, 2H), |
| | 2.22-2.10 (m, 1H), 2.03-1.89 (m, 1H), 1.87-1.73 (m, 1H), 1.77-1.58 (m, |
| | 1H), 1.45-1.26 (m, 1H), 1.20 (t, J = 7.2 Hz, 3H). NH not observed. |
| 272 | MS m/z 459.3 [M + H]⁺. ¹HNMR (400 MHz, Methanol-d₄) δ 8.82 (s, 1H), 8.22 |
| | (s, 1H), 8.16 (d, J = 8.0 Hz, 1H), 7.92 (d, J = 8.0 Hz, 1H), 7.53 (s, 1H), 3.69- |
| | 3.38 (m, 2H), 3.04 (s, 3H), 2.48-2.35 (m, 1H), 2.26-1.87 (m, 3H). NH not |
| | observed. 1H alpha to carbonyl and nitrogen linker is overlapping with water |
| | peak. |
| 273 | MS m/z 448.2 [M + H]⁺. ¹HNMR (500 MHz, Methanol-d₄) δ 8.85 (s, 1H), 8.22 |
| | (s, 1H), 8.16 (d, J = 8.1 Hz, 1H), 7.92 (d, J = 8.0 Hz, 1H), 7.56-7.40 (m, 1H), |
| | 4.42-4.25 (m, 1H), 4.13-4.03 (m, 1H), 4.02-3.95 (m, 1H), 3.84-3.70 (m, |
| | 1H), 3.63-3.49 (m, 1H), 3.32-3.24 (m, 1H), 2.41-2.14 (m, 1H), 1.90-1.65 |
| | (m, 1H). NH and OH not observed. |
| 274 | MS m/z 448.2 [M + H]⁺. ¹HNMR (400 MHz, Methanol-d₄) δ 8.85 (s, 1H), 8.22 |
| | (s, 1H), 8.16 (d, J = 8.0 Hz, 1H), 7.91 (d, J = 8.0 Hz, 1H), 7.47 (s, 1H), 4.44- |
| | 4.15 (m, 2H), 4.07-3.97 (m, 1H), 3.97-3.91 (m, 1H), 3.61-3.50 (m, 1H), |
| | 3.41-3.34 (m, 1H), 2.16-2.11 (m, 1H), 1.85-1.70 (m, 1H). NH and OH not |
| | observed. |
| 275 | MS m/z 446.3 [M + H]⁺. ¹HNMR (400 MHz, Methanol-d₄) δ 8.88 (s, 1H), 8.22 |
| | (s, 1H), 8.15 (d, J = 8.0 Hz, 1H), 7.92 (d, J = 8.0 Hz, 1H), 7.44 (s, 1H), 4.23 (p, |
| | J = 7.3 Hz, 1H), 4.06-3.98 (m, 1H), 2.05-1.87 (m, 6H), 1.85-1.69 (m, 2H). |
| | NH and OH not observed. |
| 276 | MS m/z 445.2 [M + H]⁺. ¹HNMR (400 MHz, Methanol-d₄) δ 8.77 (s, 1H), 8.22 |
| | (s, 1H), 8.17 (d, J = 8.8 Hz, 1H), 7.92 (d, J = 8.0 Hz, 1H), 7.46 (s, 1H), 3.51- |
| | 3.40 (m, 3H), 2.48-2.36 (m, 1H), 2.25-1.88 (m, 3H). 2 NH not observed. |
| 277 | MS m/z 439.3 [M + H]⁺. ¹HNMR (400 MHz, Methanol-d₄) δ 8.82 (br s, 1H), |
| | 7.60 (br s, 1H), 7.53 (d, J = 8.5 Hz, 1H), 7.13 (s, 1H), 7.11 (d, J = 8.5 Hz, 1H), |
| | 5.09 (d, J = 45.7 Hz, 1H), 4.81-4.70 (m, 1H), 3.90 (s, 3 H), 3.78 (q, J = 11.0 |
| | Hz, 2H), 3.45 (d, J = 10.0 Hz, 1H), 3.24 (t, J = 11.9 Hz, 1H), 2.68 (dd, J = 35.1, |
| | 14.4 Hz, 1H), 2.55 (s, 3H), 2.52-2.42 (m, 2H), 2.06-1.82 (m, 1H). 1H not |
| | observed (NH). |
| 278 | MS m/z 451.2 [M + H]⁺. ¹HNMR (400 MHz, Methanol-d₄) δ 8.76 (s, 1H, |
| | formic acid), 8.45 (s, 1H), 7.53 (d, J = 8.5 Hz, 1H), 7.46 (s, 1H), 7.13 (s, 1H), |
| | 7.13-7.09 (m, 1H), 4.59-4.41 (m, 1H), 3.90 (s, 3H), 3.88-3.74 (m, 4H), |
| | 3.63-3.44 (m, 1H), 3.23-3.10 (m, 1H), 3.07-2.92 (m, 2H), 2.92-2.66 (m, |
| | 2H), 2.23-2.11 (m, 1H), 2.10-2.00 (m, 1H), 1.96-1.69 (m, 2H). 2H not |
| | observed (NH and OH). |
| 279 | MS m/z 473.3 [M + H]⁺. ¹HNMR (400 MHz, Methanol-d₄) δ 8.71 (s, 1H), 8.44- |
| | 8.63 (m, 1H), 7.96 (s, 1H), 7.87 (s, 1H), 7.60-7.72 (m, 1H), 4.31-4.59 (m, 1H), |
| | 3.79-4.02 (m, 1H), 3.51-3.73 (m, 1H), 3.34-3.42 (m, 1H), 2.88-3.06 (m, 1H), |
| | 2.56 (s, 5H), 2.10-2.28 (m, 1H), 1.96 (s, 4H), 1.77-1.89 (m, 1H), 1.56-1.77 (m, |
| | 1H). NH not observed. |
| 280 | MS m/z 474.3 [M + H]⁺. ¹HNMR (400 MHz, Methanol-d₄) δ: 8.60 (s, 1H), 7.84 |
| | (s, 1H), 7.74-7.79 (m, 1H), 7.51-7.59 (m, 1H), 3.94-4.10 (m, 1H), 3.69-3.94 |
| | (m, 1H), 3.45-3.61 (m, 2H), 2.07-2.22 (m, 1H), 1.98-2.07 (m, 1H), 1.85 (s, |
| | 3H), 1.61-1.77 (m, 2H), 1.24-1.40 (m, 4H). NH and OH not observed. |
| 281 | MS m/z 457.3 [M + H]+; ¹HNMR (400 MHz, Methanol-d₄) δ: 8.75 (s, 1H), |
| | 8.45-8.62 (m, 1H), 7.49 (s, 1H), 7.36-7.43 (m, 1H), 7.31 (s, 2H), 4.34-4.63 (m, |
| | 1H), 3.96 (s, 3H), 2.89-3.06 (m, 1H), 2.33-2.74 (ms, 5H), 2.08-2.23 (m, 1H), |
| | 1.91-2.07 (m, 1H), 1.79-1.90 (m, 2H), 1.63-1.76 (m, 1H). NH not observed. |
| 282 | MS m/z 460.1 [M + H]+; ¹HNMR (400 MHz, DMSO-d₆) δ 8.88 (s, 1H), 8.00- |
| | 7.96 (m, 1H), 7.93-7.82 (m, 3H), 7.49 (s, 1H), 4.87 (t, J = 5.2 Hz, 1H), 4.14 |
| | (dd, J = 23.7, 10.8 Hz, 3H), 3.54 (d, J = 9.1 Hz, 1H), 2.16-1.93 (m, 2H), 1.81- |
| | 1.62 (m, 2H), 1.44-1.24 (m, 4H). |
| 283 | MS m/z 474.0 [M + H]+; ¹HNMR (400 MHz, DMSO-d₆) δ 9.07-9.01 (m, 1H), |
| | 7.99 (s, 1H), 7.88 (t, J = 9.6 Hz, 2H), 7.55-7.46 (m, 1H), 7.37-7.30 (m, 1H), |
| | 4.72 (s, 1H), 4.43 (s, 1H), 4.18-4.03 (m, 2H), 1.97-1.89 (m, 1H), 1.73 (s, |
| | 2H), 1.67-1.47 (m, 3H), 1.37 (t, J = 11.4 Hz, 2H), 1.23 (s, 3H). |
| 284 | MS m/z 409.0 [M + H]+; ¹HNMR (400 MHz, DMSO-d₆) δ 8.81 (s, 1H), 8.36 (d, |
| | J = 7.8 Hz, 1H), 8.05 (s, 1H), 7.66 (d, J = 8.6 Hz, 1H), 7.46 (s, 1H), 7.17 (dd, |
| | J = 8.7, 2.4 Hz, 1H), 7.10 (s, 1H), 4.94 (dd, J = 17.9, 8.5 Hz, 1H), 3.89 (s, 3H), |
| | 3.31-3.20 (m, 2H), 2.61-2.55 (m, 1H), 2.22-2.09 (m, 1H). |
| 285 | MS m/z 414.0 [M + H]+; ¹HNMR (400 MHz, DMSO-d₆) δ 8.81 (s, 1H), 8.29 (d, |
| | J = 9.8 Hz, 2H), 8.07 (d, J = 7.8 Hz, 1H), 7.77 (s, 1H), 7.43 (s, 1H), 6.22 (s, |
| | 1H). |
| 286 | MS m/z 406.3 [M + H]+; ¹HNMR (400 MHz, Methanol-d₄) δ 8.86-8.70 (m, |
| | 1H), 7.48 (d, J = 7.9 Hz, 1H), 7.44-7.39 (m, 2H), 7.38-7.33 (m, 1H), 4.21- |
| | 4.06 (m, 1H), 3.86-3.69 (m, 2H), 3.69-3.60 (m, 1H), 2.45 (s, 3H), 2.25 (d, |
| | J = 4.1 Hz, 1H), 2.15-2.04 (m, 1H), 1.89-1.75 (m, 2H), 1.55-1.35 (m, 4H). |
| | NH and OH peak not observed. |
| 287 | MS m/z 412.9, 414.9 [M + H]⁺. ¹HNMR (400 MHz, DMSO-d₆) δ 8.83 (s, 1H), |
| | 8.48 (brs, 1H), 8.05 (s, 1H), 7.79-7.76 (m, 1H), 7.76-7.73 (m, 1H), 7.70- |
| | 7.66 (m, 1H), 7.54-7.48 (m, 1H), 5.02-4.88 (m, 1H), 3.34-3.31 (m, 2H), |
| | 2.60-2.55 (m, 1H), 2.20-2.10 (m, 1H). |
| 288 | MS m/z 391.3 [M + H]⁺. 1H NMR (400 MHz, Methanol-d₄) δ 8.77-8.63 (m, |
| | 1H), 7.52-7.39 (m, 3H), 7.39-7.30 (m, 1H), 5.15-4.97 (m, 1H), 3.74 (q, J = |
| | 11.0 Hz, 2H), 3.58-3.42 (m, 2H), 2.84-2.67 (m, 1H), 2.46 (s, 3H), 2.40- |
| | 2.26 (m, 1H). 2 NH peak not observed. |
| 289 | MS m/z 475.0 [M + H]⁺. ¹HNMR (400 MHz, Methanol-d₄) δ 7.79 (s, 2H), 7.73 |
| | (d, J = 8.4 Hz, 1H), 7.60 (d, J = 8.0 Hz, 1H), 7.20 (s, 1H), 4.00-3.95 (m, 1H), |
| | 3.75-3.67 (m, 2H), 2.92 (dd, J = 12.0, 5.2 Hz, 1H), 2.66-2.61 (m, 1H), 2.52- |
| | 2.47 (m, 1H), 2.36 (d, J = 12.0 Hz, 5H), 2.22-2.16 (m, 2H). NH and OH not |
| | observed. |
| 290 | MS m/z 462.1 [M + H]⁺. ¹HNMR (400 MHz, Methanol-d₄) δ 8.80 (s, 1H), 7.93 |
| | (dd, J = 23.4, 8.2 Hz, 2H), 7.83 (s, 1H), 7.54 (s, 1H), 4.23-4.15 (m, 1H), 3.71- |
| | 3.62 (m, 1H), 2.29-2.20 (m, 1H), 2.17-2.07 (m, 1H), 1.88-1.77 (m, 2H), |
| | 1.50-1.41 (m, 4H). NH and OH not observed. |
| 291 | MS m/z 410.1 [M + H]⁺. ¹HNMR (400 MHz, Methanol-d₄) δ 8.73 (s, 1H), 7.63 |
| | (d, J = 8.8 Hz, 1H), 7.48 (s, 1H), 7.12 (dd, J = 2.0 Hz, J = 8.4 Hz, 1H), 7.04 (s, |
| | 1H), 4.62-4.53 (m, 1H), 3.92 (s, 3H), 3.59 (d, J = 5.6 Hz, 2H), 2.66-2.60 (m, |
| | 2H), 2.36-2.30 (m, 1H), 1.99-1.92 (m, 2H). NH and OH not observed. |
| 292 | MS m/z 426.3 [M + H]⁺. ¹HNMR (400 MHz, Methanol-d₄) δ 8.76 (s, 1H), 7.63 |
| | (d, J = 8.6 Hz, 1H), 7.49 (s, 1H), 7.13 (dd, J = 8.6, 2.1 Hz, 1H), 7.05 (s, 1H), |
| | 4.32-4.28 (m, 1H), 4.08-4.01 (m, 1H), 4.00-3.95 (m, 1H), 3.92 (s, 3H), 3.76 |
| | (td, J = 9.7, 4.8 Hz, 1H), 3.55 (t, J = 11.1 Hz, 1H), 3.30-3.20 (m, 1H), 2.28- |
| | 2.22 (m, 1H), 1.86-1.68 (m, 1H). NH and OH peak not observed. |
| 293 | MS m/z 446.1 [M + H]⁺. ¹HNMR (400 MHz, DMSO-d₆) δ 8.88 (s, 1H), 8.26 (s, |
| | 1H), 8.23 (d, J = 8.3 Hz, 1H), 7.96 (d, J = 8.1 Hz, 1H), 7.86 (d, J = 7.8 Hz, 1H), |
| | 7.41 (s, 1H), 4.87 (d, J = 4.9 Hz, 1H), 4.16-3.96 (m, 1H), 3.63-3.48 (m, 1H), |
| | 2.16-2.06 (m, 1H), 2.03-1.93 (m, 1H), 1.78-1.61 (m, 2H), 1.41-1.27 (m, |
| | 4H). |
| 294 | MS m/z 431.0 [M + H]⁺. ¹HNMR (400 MHz, Methanol-d₄) δ 8.72 (s, 1H), 8.20 |
| | (s, 1H), 8.14 (d, J = 8.2 Hz, 1H), 7.90 (d, J = 8.1 Hz, 1H), 7.43 (s, 1H), 5.16- |
| | 5.02 (m, 1H), 3.57-3.42 (m, 2H), 2.83-2.66 (m, 1H), 2.43-2.22 (m, 1H). |
| | NH not observed. |
| 295 | MS m/z 459.1 [M + H]⁺. ¹HNMR (400 MHz, DMSO-d₆) δ 8.88 (s, 1H), 7.81 (d, |
| | J = 8.5 Hz, 2H), 7.49-7.39 (m, 3H), 7.47 (t, J = 73.7 Hz, 1H), 4.37-4.24 (m, |
| | 1H), 3.06 (d, J = 6.7 Hz, 1H), 2.69 (d, J = 11.4 Hz, 1H), 2.22 (s, 3H), 2.06- |
| | 1.99 (m, 1H), 1.99-1.89 (m, 2H), 1.84-1.71 (m, 1H), 1.66-1.54 (m, 1H), |
| | 1.47-1.36 (m, 1H). |
| 296 | MS m/z 428.0 [M + H]⁺. ¹HNMR (400 MHz, DMSO-d₆) δ 12.78 (s, 1H), 12.65 |
| | (s, 1H), 8.79 (s, 1H), 7.86 (d, J = 8.4 Hz, 1H), 7.78 (s, 1H), 7.49 (t, J = 74 Hz, |
| | 1H), 7.52-7.44 (m, 4H). |
| 297 | MS m/z 428.3 [M + H]⁺. ¹HNMR (500 MHz, Methanol-d₄) δ 8.89-8.82 (m, |
| | 1H), 7.48-7.41 (m, 2H), 7.39 (s, 1H), 7.34 (d, J = 7.9 Hz, 1H), 7.16 (t, J = 7.9 |
| | Hz, 1H), 6.97 (d, J = 7.8 Hz, 1H), 6.94 (s, 1H), 6.68 (d, J = 8.1 Hz, 1H), 5.54- |
| | 5.39 (m, 1H), 3.88-3.60 (m, 2H), 2.44 (s, 3H), 1.74-1.66 (m, 3H). NH and |
| | OH peak not observed. |
| 298 | MS m/z 398.0, 400.0 [M + H]⁺. ¹HNMR (400 MHz, Methanol-d₄) δ 8.76 (s, |
| | 1H), 7.94 (d, J = 2.0 Hz, 1H), 7.82 (dd, J = 8.3, 1.9 Hz, 1H), 7.65 (d, J = 8.2 |
| | Hz, 1H), 7.37 (s, 1H), 4.26 (s, 1H), 2.73-2.59 (m, 2H), 2.28 (dd, J = 11.4, 9.1 |
| | Hz, 2H), 1.44 (s, 3H). 2H not observed (NH and OH). |
| 299 | MS m/z 414.1, 416.1 [M + H]⁺. ¹HNMR (400 MHz, DMSO-d₆) δ 8.83 (s, 1H), |
| | 8.25 (d, J = 6.1 Hz, 1H), 7.76 (t, J = 6.3 Hz, 2H), 7.68 (dd, J = 8.3, 1.9 Hz, 1H), |
| | 7.48 (s, 1H), 5.12 (s, 1H), 4.22 (dt, J = 15.2, 7.7 Hz, 1H), 2.48 (d, J = 2.9 Hz, |
| | 2H), 2.21 (t, J = 10.1 Hz, 2H), 1.34 (s, 3H). |
| 300 | MS m/z 428.2, 430.2 [M + H]⁺. ¹HNMR (400 MHz, DMSO-d₆) δ 8.86 (s, 1H), |
| | 7.86 (d, J = 7.8 Hz, 1H), 7.79-7.72 (m, 2H), 7.68 (dd, J = 8.3, 1.6 Hz, 1H), 7.47 |
| | (s, 1H), 4.94-4.83 (m, 1H), 4.13-3.99 (m, 1H), 3.61-3.50 (m, 1H), 2.14- |
| | 2.06 (m, 1H), 2.03-1.94 (m, 1H), 1.76-1.66 (m, 2H), 1.41-1.22 (m, 4H). |
| 301 | MS m/z 489.1 [M + H]⁺. ¹HNMR (400 MHz, DMSO-d₆) δ 8.88 (s, 1H), 8.17 (s, |
| | 1H), 7.99 (s, 1H), 7.89 (dd, J = 19.9, 8.1 Hz, 2H), 7.49 (s, 1H), 4.31 (s, 1H), 4.18- |
| | 4.04 (m, 2H), 3.53 (t, J = 6.1 Hz, 2H), 3.19 (s, 2H), 2.83 (d, J = 12.3 Hz, 1H), |
| | 2.47 (d, J = 6.2 Hz, 2H), 2.16-2.01 (m, 3H), 1.75 (s, 1H), 1.60 (d, J = 12.1 Hz, |
| | 1H), 1.46 (s, 1H). |
| 302 | MS m/z 422.1 [M + H]⁺. ¹HNMR (400 MHz, Methanol-d₄) δ 8.80 (br s, 1H), |
| | 7.55 (d, J = 8.4 Hz, 1H), 7.52 (br s, 1H), 7.13 (s, 1H), 7.11 (d, J = 8.5 Hz, 1H), |
| | 4.24-4.15 (m, 1H), 3.90 (s, 3H), 3.86-3.69 (m, 3H), 2.47 (d, J = 11.6 Hz, |
| | 1H), 2.17 (d, J = 11.7 Hz, 1H), 2.01 (d, J = 11.4 Hz, 1H), 1.90 (d, J = 13.6 Hz, |
| | 1H), 1.54-1.34 (m, 3H), 1.32-1.22 (m, 1H). 2H not observed (NH and OH). |
| 303 | MS m/z 444.2 [M + H]⁺. ¹HNMR (500 MHz, DMSO-d₆) δ 9.34 (br s, 1H), 8.95 |
| | (s, 1H), 8.20 (d, J = 7.48 Hz, 1H), 7.53 (d, J = 8.5 Hz, 1H), 7.41 (s, 1H), 7.17- |
| | 7.05 (m, 3H), 6.90 (d, J = 7.5 Hz, 1H), 6.88 (s, 1H), 6.63 (d, J = 8.09 Hz, 1H), |
| | 5.43 (quint, J = 7.32 Hz, 1H), 4.08-3.94 (m, 1H), 3.88-3.77 (m, 4H), 1.60 |
| | (d, J = 6.9 Hz, 3H). |
| 304 | MS m/z 437.1 [M + H]⁺. ¹HNMR (400 MHz, Methanol-d₄) δ 8.69 (s, 1H), 7.71- |
| | 7.24 (m, 2H), 6.82-6.74 (m, 2H), 4.57 (q, J = 8.5 Hz, 2H), 4.42 (s, 1H), 3.88 |
| | (s, 3H), 3.20-3.07 (m, 1H), 2.77-2.64 (m, 1H), 2.33 (s, 3H), 2.31-1.99 (m, |
| | 3H), 1.95-1.81 (m, 1H), 1.80-1.67 (m, 1H), 1.67-1.51 (m, 1H). NH not |
| | observed. |
| 305 | MS m/z 424.1 [M + H]⁺. ¹HNMR (400 MHz, Methanol-d₄) δ 8.67 (s, 1H), 7.52- |
| | 7.41 (m, 2H), 6.82-6.74 (m, 2H), 4.57 (q, J = 8.5 Hz, 2H), 4.30-4.20 (m, |
| | 1H), 3.88 (s, 3H), 2.72-2.60 (m, 2H), 2.33-2.18 (m, 2H), 1.43 (s, 3H). NH |
| | and OH not observed. |
| 306 | MS m/z 453.9, 455.9 [M + H]+; ¹HNMR (400 MHz, Methanol-d₄) δ 8.75 (s, |
| | 1H), 7.62-7.53 (m, 3H), 7.49 (s, 1H), 6.89 (t, J = 73.2Hz, 1H), 4.23-4.10 (m, |
| | 1H), 3.71-3.58 (m, 1H), 2.29-2.06 (m, 2H), 1.87-1.75 (m, 2H), 1.50-1.37 |
| | (m, 4H). NH and OH not observed. |
| 307 | MS m/z 472.1, 474.1 [M + H]⁺. ¹HNMR (400 MHz, Methanol-d₄) δ 8.77 (s, 1H), |
| | 7.75 (d, J = 8.0, 2H), 7.64 (d, J = 8.0, 1H), 7.51 (s, 1H), 4.15-4.17 (m, |
| | 1H), 3.62-3.64 (m, 1H), 2.10-2.23 (m, 2H), 1.80-1.81 (m, 2H), 1.43-1.48 |
| | (m, 4H). NH and OH not observed. |
| 308 | MS m/z 431.1 [M + H]⁺. ¹HNMR (400 MHz, Methanol-d₄) δ 8.83 (s, 1H), 8.71 |
| | (d, J = 1.6 Hz, 1H), 8.44 (d, J = 4.0 Hz, 1H), 8.01 (d, J = 7.6 Hz, 1H), 7.59 (d, |
| | J = 8.8 Hz, 1H), 7.51 (s, 1H), 7.45-7.41 (m, 1H), 7.10 (dd, J = 2.4Hz, J = 8.8 |
| | Hz, 1H), 7.02 (s, 1H), 5.58-5.53 (m, 1H), 3.90 (s, 3H), 1.76 (d, J = 7.2 Hz, |
| | 3H). NH not observed. |
| 309 | MS m/z 431.0 [M + H]⁺. ¹HNMR (400 MHz, Methanol-d₄) δ 8.85 (s, 1H), |
| | 8.72(s, 1H), 8.44 (d, J = 4.0 Hz, 1H), 8.01 (d, J = 8.0 Hz, 1H), 7.59 (d, J = 8.8 |
| | Hz, 1H), 7.50 (s, 1H), 7.45-7.42 (m, 1H), 7.10 (dd, J = 2.4 Hz, J = 8.8 Hz, |
| | 1H), 7.02 (s, 1H), 5.56-5.54 (m, 1H), 3.90 (s, 3H), 1.76(d, J = 7.2 Hz, 3H). |
| | NH not observed. |
| 310 | MS m/z 438.1 [M + H]⁺. ¹HNMR (400 MHz, Methanol-d₄) δ 8.86 (s, 1H), 7.62 |
| | (d, J = 8.8 Hz, 1H), 7.49 (s, 1H), 7.12 (dd, J = 8.4, J = 2.0 Hz, 1H), 7.04 (s, |
| | 1H), 4.58-4.33 (m, 1H), 3.91 (s, 3H), 2.07-1.96 (m, 1H), 1.88-1.79 (m, 2H), |
| | 1.77-1.60 (m, 3H), 1.53-1.42 (m, 2H), 1.31 (s, 3H). NH and OH not observed. |
| 311 | MS m/z 438.1 [M + H]+; ¹HNMR (400 MHz, Methanol-d₄) δ 8.86 (s, 1H), 7.62 |
| | (d, J = 8.8Hz, 1H), 7.48 (s, 1H), 7.12 (dd, J = 8.4, J = 2.4 Hz, 1H), 7.04 (s, 1H), |
| | 4.51-4.31 (m, 1H), 3.91 (s, 3H), 2.06-1.95 (m, 1H), 1.91-1.80 (m, 2H), 1.76- |
| | 1.59 (m, 3H), 1.54-1.41 (m, 2H), 1.31 (s, 3H). NH and OH not observed. |
| 312 | MS m/z 471.1, 473.1 [M + H]⁺. ¹HNMR (400 MHz, Methanol-d₄) δ 8.76 (s, |
| | 1H), 7.78-7.71 (m, 2H), 7.65 (d, J = 8.4Hz, 1H), 7.52 (s, 1H), 4.45 (s, |
| | 1H), 3.12-3.17(m, 1H), 2.73-2.75 (m, 1H), 2.37 (s, 3H), 2.28-2.30 (m, 2H), |
| | 2.09-2.11 (m, 1H), 1.86-1.88 (m, 1H), 1.74-1.7 7 (m, 1H), 1.60-1.63 (m, |
| | 1H). NH not observed. |
| 313 | MS m/z 462.2 [M + H]⁺. ¹HNMR (500 MHz, DMSO-d₆) δ 8.88 (s, 1H), 8.04- |
| | 7.95 (m, 2H), 7.94-7.85 (m, 2H), 7.52 (s, 1H), 5.26-5.16 (m, 1H), 4.35- |
| | 4.21 (m, 1H), 4.21-4.03 (m, 2H), 3.98-3.82 (m, 2H), 3.71-3.62 (m, 1H), |
| | 3.43 (t, J = 11.7 Hz, 1H), 3.13 (t, J = 10.5 Hz, 1H), 2.13 (d, J = 11.1 Hz, 1H), |
| | 1.70-1.59 (m, 1H). |
| 314 | MS m/z 408.3 [M + H]⁺. ¹HNMR (500 MHz, DMSO-d₆) δ 8.83 (br s, 1H), 8.03 |
| | (d, J = 6.4 Hz, 1H), 7.54 (d, J = 8.4 Hz, 1H), 7.44 (br s, 1H), 7.13 (s, 1H), 7.09 |
| | (d, J = 9.5 Hz, 1H), 4.69-4.47 (m, 2H), 3.94 (q, J = 11.4 Hz, 2H), 3.84 (s, |
| | 3H), 3.43 (d, J = 5.5 Hz, 2H), 2.47-2.35 (m, 2H), 2.23-2.13 (m, 1H), 1.90 |
| | (q, J = 9.6 Hz, 2H). |
| 315 | MS m/z 444.3, [M + H]⁺. ¹HNMR (500 MHz, Methanol-d₄) δ 8.55-8.69 (m, |
| | 1H), 7.335-7.40 (m, 1H), 7.22-7.32 (m, 1H), 7.19 (s, 1H), 7.13 (s, 1H), 4.15 |
| | (quin, J = 8.0 Hz, 1H), 3.84 (s, 3H), 2.48-2.66 (m, 2H), 2.05-2.33 (m, 2H), 1.34 |
| | (s, 3H). NH and OH not observed. |
| 316 | MS m/z 458.3, [M + H]⁺. ¹HNMR (400 MHz, Methanol-d₄) δ: 8.64 (s, 1H), 7.37 |
| | (s, 1H), 7.24-7.28 (m, 1H), 7.19 (s, 1H), 7.10-7.15 (m, 1H), 3.96-4.13 (m, 1H), |
| | 3.84 (s, 3H), 3.50-3.59 (m, 1H), 2.11-2.19 (m, 1H), 1.95-2.06 (m, 1H), 1.67- |
| | 1.72 (m, 2H), 1.26-1.50 (m, 4H). NH and OH not observed. |
| 317 | MS m/z 405.3 [M + H]⁺. ¹HNMR (400 MHz, Methanol-d₄) δ 8.81-8.70 (m, |
| | 1H), 7.53-7.48 (m, 1H), 7.43-7.39 (m, 2H), 7.39-7.34 (m, 1H), 4.54- |
| | 4.36 (m, 1H), 3.83-3.67 (m, 2H), 3.61-3.45 (m, 1H), 3.04-2.88 (m, 1H), |
| | 2.65-2.51 (m, 5H), 2.45 (s, 3H), 2.20-2.09 (m, 1H), 2.06-1.94 (m, 1H), |
| | 1.90-1.79 (m, 1H), 1.76-1.64 (m, 1H). NH peak not observed. |
| 318 | MS m/z 392.3 [M + H]⁺. ¹HNMR (500 MHz, Methanol-d₄) δ 8.73 (s, 1H), 7.51- |
| | 7.46 (m, 1H), 7.45-7.39 (m, 2H), 7.38-7.33 (m, 1H), 4.29-4.24 (m, 1H), |
| | 3.77 (q, J = 11.1 Hz, 2H), 2.72-2.61 (m, 2H), 2.45 (s, 3H), 2.31-2.23 (m, |
| | 2H), 1.44 (s, 3H). NH and OH peak not observed. |
| 319 | MS m/z 366.3 [M + H]⁺. ¹HNMR (400 MHz, Methanol-d₄) δ 8.71 (s, 1H), 7.41 |
| | (s, 1H), 7.32 (d, J = 8.4 Hz, 1H), 6.87 (dd, J = 8.4, 2.1 Hz, 1H), 6.61 (d, J = 1.9 |
| | Hz, 1H), 4.27 (quint, J = 8.0 Hz, 1H), 3.84 (s, 3H), 2.71-2.60 (m, 2H), 2.33- |
| | 2.20 (m, 2H), 1.94-1.85 (m, 1H), 1.44 (s, 3H), 0.83-0.74 (m, 2H), 0.71- |
| | 0.62 (m, 2H). NH and OH not observed. |
| 320 | MS m/z 365.3 [M + H]⁺. ¹HNMR (400 MHz, Methanol-d₄) δ 8.67 (s, 1H), 7.44 |
| | (s, 1H), 7.33 (d, J = 8.4 Hz, 1H), 6.88 (d, J = 8.3 Hz, 1H), 6.60 (s, 1H), 5.14- |
| | 4.96 (m, 1H), 3.84 (s, 3H), 3.57-3.43 (m, 2H), 2.80-2.65 (m, 1H), 2.44- |
| | 2.26 (m, 1H), 1.99-1.81 (m, 1H), 0.89-0.76 (m, 2H), 0.72-0.58 (m, 2H). |
| | NH not observed. |
| 321 | MS m/z 380.3 [M + H]⁺. 1HINMR (400 MHz, Methanol-d₄) δ 8.74 (s, 1H), 7.40 |
| | (s, 1H), 7.32 (d, J = 8.4 Hz, 1H), 6.88 (dd, J = 8.4, 2.1 Hz, 1H), 6.60 (d, J = 1.9 |
| | Hz, 1H), 4.14 (s, 1H), 3.84 (s, 3H), 3.65 (d, J = 9.6 Hz, 1H), 2.26 (s, 1H), 2.12 |
| | (d, J = 10.2 Hz, 1H), 1.91 (d, J = 8.8 Hz, 1H), 1.82 (d, J = 16.8 Hz, 2H), 1.44 |
| | (h, J = 12.4 Hz, 4H), 0.78 (d, J = 8.0 Hz, 2H), 0.66 (d, J = 5.0 Hz, 2H). NH and |
| | OH not observed. |
| 322 | MS m/z 464.2 [M + H]⁺. ¹HNMR (400 MHz, Methanol-d₄) δ 8.80 (s, 1H), 7.96 |
| | (d, J = 8.1 Hz, 1H), 7.91 (d, J = 8.1 Hz, 1H), 7.83 (s, 1H), 7.57 (s, 1H), 4.38 (s, |
| | 1H), 4.05 (dd, J = 11.1, 5.0 Hz, 1H), 4.02-3.94 (m, 1H), 3.84-3.72 (m, 1H), |
| | 3.56 (t, J = 11.9 Hz, 1H), 3.27 (t, J = 10.6 Hz, 1H), 2.38-2.18 (m, 1H), 1.85- |
| | 1.72 (m, 1H). NH and OH not observed. |
| 323 | MS m/z 484.2 [M + H]⁺. ¹HNMR (400 MHz, Methanol-d₄) δ 8.90 (s, 1H), 7.93 |
| | (d, J = 8.1 Hz, 1H), 7.88 (d, J = 8.2 Hz, 1H), 7.82 (s, 1H), 7.56 (s, 1H), 7.16 (t, |
| | J = 7.9 Hz, 1H), 6.97 (d, J = 7.6 Hz, 1H), 6.94 (s, 1H), 6.68 (d, J = 8.0 Hz, 1H), |
| | 5.50 (q, J = 6.8 Hz, 1H), 1.71 (d, J = 6.9 Hz, 3H). NH and OH not observed. |
| 324 | MS m/z 462.2 [M + H]⁺. ¹HNMR (400 MHz, Methanol-d₄) δ 8.77 (s, 1H), 7.96 |
| | (d, J = 8.0 Hz, 1H), 7.90 (d, J = 8.1 Hz, 1H), 7.83 (s, 1H), 7.55 (s, 1H), 4.62 |
| | (quint, J = 8.3 Hz, 1H), 3.45 (d, J = 5.9 Hz, 2H), 3.37 (s, 3H), 2.71-2.60 (m, |
| | 2H), 2.47-2.34 (m, 1H), 2.09-1.94 (m, 2H). |
| 325 | MS m/z 476.2 [M + H]⁺. ¹HNMR (400 MHz, Methanol-d₄) δ 8.79 (s, 1H), 7.96 |
| | (d, J = 8.0 Hz, 1H), 7.90 (d, J = 8.1 Hz, 1H), 7.83 (s, 1H), 7.55 (s, 1H), 4.51 |
| | (quint, J = 8.2 Hz, 1H), 2.56-2.46 (m, 2H), 2.25-2.16 (m, 1H), 2.16-2.07 |
| | (m, 2H), 1.17 (s, 6H). NH and OH not observed. |
| 326 | MS m/z 422.3 [M + H]⁺. ¹HNMR (400 MHz, Methanol-d₄) δ 8.73 (s, 1H), 7.53 |
| | (d, J = 8.5 Hz, 1H), 7.44 (s, 1H), 7.12 (s, 1H), 7.09 (dd, J = 8.3, 2.1 Hz, 1H), |
| | 4.57 (quint, J = 8.1 Hz, 1H), 3.89 (s, 3H), 3.80 (q, J = 11.0 Hz, 2H), 3.45 (d, J = |
| | 6.0 Hz, 2H), 3.37 (s, 3H), 2.68-2.58 (m, 2H), 2.48-2.32 (m, 1H), 2.05-1.93 |
| | (m, 2H). |
| 327 | MS m/z 436.3 [M + H]⁺. ¹HNMR (400 MHz, Methanol-d₄) δ 8.74 (s, 1H), 7.53 |
| | (d, J = 8.5 Hz, 1H), 7.44 (s, 1H), 7.12 (s, 1H), 7.10 (dd, J = 8.6, 2.4 Hz, 1H), |
| | 4.46 (quint, J = 8.2 Hz, 1H), 3.90 (s, 3H), 3.80 (q, J = 10.9 Hz, 2H), 2.60-2.43 |
| | (m, 2H), 2.24-2.14 (m, 1H), 2.14-2.04 (m, 2H), 1.17 (s, 6H). |
| 328 | MS m/z 473.3 [M + H]⁺. ¹HNMR (500 MHz, Methanol-d₄) δ 8.73 (s, 1H), 7.78 |
| | (d, J = 8.6 Hz, 1H), 7.54 (s, 1H), 7.38 (d, J = 8.4 Hz, 1H), 7.34 (s, 1H), 7.07 (t, |
| | J = 73.0 Hz, 1H), 4.90-4.82 (m, 1H), 3.62-3.50 (m, 1H), 3.50-3.41 (m, |
| | 1H), 3.03 (s, 3H), 2.48-2.29 (m, 1H), 2.28-2.14 (m, 1H), 2.14-2.00 (m, |
| | 2H). 1 NH is missing. |
| 329 | MS m/z 473.3 [M + H]⁺. ¹HNMR (500 MHz, Methanol-d₄) δ 8.74 (s, 1H), 7.94 |
| | (s, 1H), 7.88 (d, J = 8.1 Hz, 1H), 7.84 (d, J = 8.2 Hz, 1H), 7.51 (s, 1H), 3.96 (d, |
| | J = 10.8 Hz, 1H), 3.91 (d, J = 11.0 Hz, 1H), 3.60-3.51 (m, 1H), 3.50-3.43 |
| | (m, 1H), 3.04 (s, 3H), 2.44-2.33 (m, 1H), 2.29-2.15 (m, 1H), 2.14-2.01 (m, |
| | 2H). the proton N—CH-carbonyl overlaps with the water peak. |
| 330 | MS m/z 435.3 [M + H]⁺. ¹HNMR (500 MHz, Methanol-d₄) δ 8.82 (s, 1H), 7.60 |
| | (s, 1H), 7.54 (d, J = 8.4 Hz, 1H), 7.15 (s, 1H), 7.11 (d, J = 8.2 Hz, 1H), 4.89- |
| | 4.81 (m, 1H), 3.91 (s, 3H), 3.81 (d, J = 11.2 Hz, 1H), 3.77 (d, J = 11.2 Hz, 1H), |
| | 3.61-3.50 (m, 1H), 3.50-3.41 (m, 1H), 3.03 (s, 3H), 2.55-2.32 (m, 1H), |
| | 2.30-1.95 (m, 3H). NH not observed. |
| 331 | MS m/z 462.1 [M + H]⁺. ¹HNMR (400 MHz, Methanol-d₄) δ 8.79 (br s, 1H), |
| | 7.77 (d, J = 8.5 Hz, 1H), 7.53 (br s, 1H), 7.37 (d, J = 8.8 Hz, 1H), 7.33 (s, 1H), |
| | 7.05 (t, J = 72.7 Hz, 1H), 4.40-4.27 (m, 1H), 4.04 (dd, J = 11.1, 4.8 Hz, 1H), |
| | 3.98 (dd, J = 11.6, 4.0 Hz, 1H), 3.76 (td, J = 9.5, 5.0 Hz, 1H), 3.56 (t, J = 11.7 |
| | Hz, 1H), 3.26 (t, J = 10.6 Hz, 1H), 2.26 (d, J = 12.9 Hz, 1H), 1.78 (qd, J = 12.4, |
| | 4.4 Hz, 1H). 2H not observed (NH and OH). |
| 332 | MS m/z 394.4 [M + H]⁺. ¹HNMR (500 MHz, Methanol-d₄) δ 8.85 (s, 1H), 7.81- |
| | 7.50 (m, 2H), 7.39 (d, J = 7.8 Hz, 1H), 7.36 (s, 1H), 4.53-4.11 (m, 1H), 2.74- |
| | 2.62 (m, 2H), 2.51 (s, 3H), 2.40-2.25 (m, 2H), 1.46 (s, 3H). 1 NH and 1 OH |
| | not observed. |
| 333 | MS m/z 403.2 [M + H]+; ¹HNMR (500 MHz, Methanol-d₄) δ 8.70 (s, 1H), 7.61- |
| | 7.57 (m, 2H), 7.51-7.42 (m, 1H), 7.35 (s, 1H), 5.13-5.02 (m, 1H), 3.57- |
| | 3.45 (m, 2H), 2.84-2.68 (m, 1H), 2.44-2.28 (m, 1H), 2.04-1.89 (m, 1H), |
| | 0.89 (d, J = 7.3 Hz, 2H), 0.74 (d, J = 5.2 Hz, 2H). NH peak not observed. |
| 334 | MS m/z 420.3 [M + H]⁺. ¹HNMR (500 MHz, Methanol-d₄) δ 8.78 (s, 1H), 7.63- |
| | 7.61 (m, 2H), 7.45 (s, 1H), 7.36 (s, 1H), 4.45-4.23 (m, 1H), 4.09-4.01 (m, |
| | 1H), 4.01-3.93 (m, 1H), 3.81-3.72 (m, 1H), 3.58-3.53 (m, 1H), 3.29-3.22 |
| | (m, 1H), 2.36-2.21 (m, 1H), 1.99-1.97 (m, 1H), 1.79-1.74 (m, 1H), 0.88 (d, |
| | J = 8.4 Hz, 2H), 0.74 (d, J = 5.2 Hz, 2H). NH and OH peaks were not observed. |
| 335 | MS m/z 462.2 [M + H]⁺. ¹HNMR (400 MHz, Methanol-d4) δ 8.85 (s, 1Hx2, cis |
| | and trans), 8.00-7.95 (m, 1Hx2, cis and trans), 7.94-7.89 (m, 1Hx2, cis and |
| | trans), 7.85 (s, 1Hx2, cis and trans), 7.62 (s, 1Hx2, cis and trans), 4.81-4.70 |
| | (m, 1H, cis/trans), 4.70-4.55 (m, 1H, cis/trans), 3.57 (s, 2H, cis/trans), 3.44 |
| | (s, 2H, cis/trans), 2.65-2.54 (m, 2H, cis/trans), 2.33-2.25 (m, 2H, cis/trans), |
| | 2.24-2.16 (m, 2H, cis/trans), 1.99 (t, J = 10.3 Hz, 2H, cis/trans), 1.29 (s, 3H, |
| | cis/trans), 1.24 (s, 3H, cis/trans). A mixture of cis/trans with ratio ~1:1. NH |
| | and OH not observed. |
| 336 | MS m/z 422.3 [M + H]⁺. ¹HNMR (400 MHz, Methanol-d4) δ 8.78 (s, 1H), 7.55 |
| | (d, J = 8.5 Hz, 1H), 7.48 (s, 1H), 7.14 (s, 1H), 7.12 (d, J = 10.2 Hz, 1H), 4.69- |
| | 4.54 (m, 1H), 3.91 (s, 3H), 3.81 (q, J = 11.0 Hz, 2H), 3.57 (s, 2H), 2.62-2.54 |
| | (m, 2H), 2.02-1.92 (m, 2H), 1.24 (s, 3H). NH and OH not observed. |
| 337 | MS m/z 422.3 [M + H]⁺. ¹HNMR (400 MHz, Methanol-d₄) δ 8.68 (s, 1Hx2, cis |
| | and trans), 7.49-7.28 (m, 1Hx2, cis and trans), 7.05-6.96 (m, 1Hx2, cis and |
| | trans), 4.66-4.55 (m, 1H, cis/trans), 4.54-4.43 (m, 1H, cis/trans), 3.79 (s, |
| | 3Hx2, cis and trans), 3.70 (q, J = 10.4 Hz, 4H, cis and trans), 3.45 (s, 2H, |
| | cis/trans), 3.32 (s, 2H, cis/trans), 2.51-2.42 (m, 2H, cis/trans), 2.20-2.11 (m, |
| | 2H, cis/trans), 2.11-2.02 (m, 2H, cis/trans), 1.90-1.79 (m, 2H, cis/trans), |
| | 1.17 (s, 3H, cis/trans), 1.12 (s, 3H, cis/trans). A mixture of cis/trans with |
| | ratio ~1:1. NH and OH not observed. |
| 338 | MS m/z 438.3 [M + H]⁺. ¹HNMR (400 MHz, Methanol-d₄) δ 1H NMR (400 |
| | MHz, Methanol-d₄) δ 8.76 (s, 1H), 7.63 (d, J = 8.6 Hz, 1H), 7.50 (s, 1H), 7.12 |
| | (d, J = 8.7 Hz, 1H), 7.04 (s, 1H), 4.47 (quint, J = 7.8 Hz, 1H), 3.92 (s, 3H), 2.57- |
| | 2.42 (m, 2H), 2.24-1.99 (m, 3H), 1.17 (s, 6H). NH and OH not observed. |
| 339 | MS m/z 474.2 [M + H]⁺. ¹HNMR (400 MHz, Methanol-d₄) δ 8.79 (s, 1H), 7.92 |
| | (s, 1H), 7.89-7.80 (m, 2H), 7.49 (s, 1H), 4.57-4.39 (m, 1H), 3.95 (q, J = |
| | 10.9 Hz, 2H), 2.60-2.40 (m, 2H), 2.28-2.02 (m, 3H), 1.17 (s, 6H). NH and |
| | OH not observed. |
| 340 | MS m/z 462.2 [M + H]⁺. ¹HNMR (400 MHz, Methanol-d₄) δ 8.79 (s, 1H), 7.96 |
| | (d, J = 8.1 Hz, 1H), 7.90 (d, J = 8.3 Hz, 1H), 7.83 (s, 1H), 7.55 (s, 1H), 4.80- |
| | 4.66 (m, 1H), 3.42 (s, 2H), 2.31-2.23 (m, 2H), 2.22-2.13 (m, 2H), 1.27 (s, |
| | 3H). NH and OH not observed. |
| 341 | MS m/z 422.3 [M + H]⁺. ¹HNMR (400 MHz, Methanol-d₄) δ 8.80 (s, 1H), 7.70- |
| | 7.35 (m, 2H), 7.15-7.05 (m, 2H), 4.71 (quint, J = 8.1 Hz, 1H), 3.89 (s, 3H), |
| | 3.80 (q, J = 11.0 Hz, 2H), 3.42 (s, 2H), 2.32-2.22 (m, 2H), 2.20-2.12 (m, |
| | 2H), 1.27 (s, 3H). NH and OH not observed. |
| 342 | MS m/z 424.3 [M + H]⁺. ¹HNMR (400 MHz, Methanol-d₄) δ 8.80 (s, 1H), 7.63 |
| | (d, J = 8.6 Hz, 1H), 7.55 (s, 1H), 7.12 (d, J = 8.6 Hz, 1H), 7.04 (s, 1H), 4.72 |
| | (quint, J = 8.2 Hz, 1H), 3.92 (s, 3H), 3.42 (s, 2H), 2.31-2.21 (m, 2H), 2.21- |
| | 2.12 (m, 2H), 1.26 (s, 3H). NH and OH not observed. |
| 343 | MS m/z 460.2 [M + H]⁺. ¹HNMR (400 MHz, Methanol-d₄) δ 8.80 (s, 1H), 7.92 |
| | (s, 1H), 7.89-7.78 (m, 2H), 7.51 (s, 1H), 4.74 (quint, J = 7.9 Hz, 1H), 3.95 (q, |
| | J = 10.9 Hz, 2H), 3.43 (s, 2H), 2.31-2.23 (m, 2H), 2.23-2.14 (m, 2H), 1.27 |
| | (s, 3H). NH and OH not observed. |
| 344 | MS m/z 375.3 [M + H]⁺. ¹HNMR (500 MHz, Methanol-d₄) δ 8.61 (s, 1H), 7.35- |
| | 7.42 (m, 2H), 7.03-7.16 (3, 2H), 6.46-6.93 (m, 1H), 4.15 (quin, J = 7.9 Hz, 1H), |
| | 2.46-2.72 (m, 2H), 2.37 (s, 3H), 2.00-2.23 (m, 2H), 1.34 (s, 3H). NH and OH |
| | not observed. |
| 345 | MS m/z 402.3, [M + H]⁺. ¹HNMR (500 MHz, Methanol-d₄) δ 8.51-8.76 (m, |
| | 1H), 7.29-7.50 (m, 2H), 6.94-7.08 (m, 2H), 6.44-6.91 (m, 1H), 4.15 (quin, |
| | J = 7.9 Hz, 1H), 2.45-2.67 (m, 2H), 2.08-2.27 (m, 2H), 1.85-2.05 (m, 1H), 1.34 |
| | (s, 3H), 0.90-1.11 (m, 2H), 0.61-0.78 (m, 2H). NH and OH not observed. |
| 346 | MS m/z 420.5 [M + H]⁺. ¹HNMR (500 MHz, Methanol-d₄) δ 8.86 (s, 1H), 7.87- |
| | 7.48 (m, 2H), 7.25 (d, J = 7.9 Hz, 1H), 7.22 (s, 1H), 4.41-4.10 (m, 1H), 2.80- |
| | 2.56 (m, 2H), 2.45-2.21 (m, 2H), 2.19-1.93 (m, 1H), 1.46 (s, 3H), 1.20- |
| | 1.04 (m, 2H), 0.98-0.71 (m, 2H). 1 NH and 1 OH not observed. |
| 347 | MS m/z 394.6 [M + H]⁺. ¹HNMR (500 MHz, Methanol-d₄) δ 8.86 (s, 1H), 7.96- |
| | 7.46 (m, 2H), 7.39 (d, J = 7.7 Hz, 1H), 7.36 (s, 1H), 4.68-4.49 (m, 1H), 3.63- |
| | 3.58 (m, 2H), 2.82-2.59 (m, 2H), 2.50 (s, 3H), 2.42-2.26 (m, 1H), 2.13- |
| | 1.86 (m, 2H). 1 NH and 1 OH not observed. |
| 348 | MS m/z 418.3 [M + H]⁺. ¹HNMR (400 MHz, Methanol-d₄) δ 8.77 (br s, 1H), |
| | 7.56-7.39 (m, 2H), 7.29 (s, 1H), 7.22 (d, J = 8.0 Hz, 1H), 4.25 (quint, J = 8.0 |
| | Hz, 1H), 3.77 (q, J = 11.1 Hz, 2H), 2.70-2.63 (m, 2H), 2.32-2.24 (m, 2H), |
| | 2.06-1.98 (m, 1H), 1.44 (s, 3H), 1.11-1.04 (m, 2H), 0.83-0.76 (m, 2H). 2H |
| | not observed (NH and OH). |
| 349 | MS m/z 418.3 [M + H]⁺. ¹HNMR (400 MHz, Methanol-d₄) δ 8.76 (br s, 1H), |
| | 7.56-7.39 (m, 2H), 7.29 (s, 1H), 7.22 (d, J = 7.88 Hz, 1H), 4.57 (quint, J = 8.1 |
| | Hz, 1H), 3.77 (q, J = 10.9 Hz, 2H), 3.59 (d, J = 6.0 Hz, 2H), 2.69-2.58 (m, |
| | 2H), 2.44-2.26 (m, 1H), 2.06-1.92 (m, 3H), 1.07 (q, J = 6.2 Hz, 2H), 0.86- |
| | 0.76 (m, 2H). 2H not observed (NH and OH). |
| 350 | MS m/z 417.3 [M + H]⁺. ¹HNMR (400 MHz, Methanol-d₄) δ 8.68 (br s, 1H), |
| | 7.50-7.41 (m, 2H), 7.30 (s, 1H), 7.22 (d, J = 7.88 Hz, 1H), 5.06 (t, J = 9.6 |
| | Hz, 1H), 3.75 (q, J = 11.0 Hz, 2H), 3.60-3.39 (m, 2H), 2.81-2.69 (m, 1H), |
| | 2.42-2.31 (m, 1H), 2.07-1.98 (m, 1H), 1.12-1.03 (m, 2H), 0.82-0.76 (m, |
| | 2H). 2H not observed (2 NH). |
| 351 | MS m/z 434.3 [M + H]⁺. ¹HNMR (400 MHz, Methanol-d₄) δ 8.77 (br s, 1H), |
| | 7.50-7.40 (m, 2H), 7.29 (s, 1H), 7.22 (d, J = 8.1 Hz, 1H), 4.39-4.23 (m, 1H), |
| | 4.04 (dd, J = 11.1, 4.9 Hz, 1H), 3.98 (dd, J = 11.6, 3.9 Hz, 1H), 3.85-3.67 (m, |
| | 3H), 3.55 (t, J = 11.3 Hz, 1H), 3.26 (t, J = 10.3 Hz, 1H), 2.26 (br d, J = 12.5 Hz, |
| | 1H), 2.11-1.93 (m, 1H), 1.78 (qd, J = 12.4, 4.5 Hz, 1H), 1.11-1.04 (m, 2H), |
| | 0.86-0.74 (m, 2H). 2H not observed (NH and OH). |
| 352 | MS m/z 431.3 [M + H]⁺. ¹HNMR (400 MHz, Methanol-d₄) δ 8.75 (br s, 1H), |
| | 7.49-7.38 (m, 2H), 7.29 (s, 1H), 7.23 (d, J = 7.9 Hz, 1H), 4.57-4.34 (m, 1H), |
| | 3.76 (q, J = 11.1 Hz, 2H), 3.43-3.32 (m, 1H), 3.09-2.92 (m, 1H), 2.71-2.42 |
| | (m, 5H), 2.26-2.06 (m, 1H), 2.06-1.94 (m, 2H), 1.91-1.62 (m, 2H), 1.13- |
| | 1.03 (m, 2H), 0.82-0.76 (m, 2H). 1H not observed (NH). |
| 353 | MS m/z 432.3 [M + H]⁺. ¹HNMR (400 MHz, Methanol-d₄) δ 8.74 (br s, 1H), |
| | 7.54-7.39 (m, 2H), 7.29 (s, 1H), 7.22 (d, J = 8.0 Hz, 1H), 4.27-4.13 (m, 1H), |
| | 3.83-3.69 (m, 3H), 2.47 (br d, J = 11.3 Hz, 1H), 2.17 (br d, J = 12.1 Hz, 1H), |
| | 2.06-1.96 (m, 2H), 1.96-1.84 (m, 1H), 1.55-1.20 (m, 4H), 1.11-1.03 (m, |
| | 2H), 0.83-0.76 (m, 2H). 2H not observed (NH and OH). |
| 354 | MS m/z 454.3 [M + H]⁺. ¹HNMR (400 MHz, Methanol-d₄) δ 8.93 (br s, 1H), |
| | 7.47-7.47 (m, 2H), 7.27 (s, 1H), 7.23-7.13 (m, 2H), 6.97 (d, J = 7.9 Hz, 1H), |
| | 6.93 (s, 1H), 6.68 (d, J = 7.9 Hz, 1H), 5.53-5.39 (m, 1H), 3.85-3.61 (m, 2H), |
| | 2.05-1.97 (m, 1H), 1.70 (d, J = 6.8 Hz, 3H), 1.12-1.00 (m, 2H), 0.82-0.75 |
| | (m, 2H). 2H not observed (NH and OH). |
| 355 | MS m/z 437.1 [M + H]⁺. ¹HNMR (500 MHz, Methanol-d₄) δ 8.95 (s, 1H), 8.10- |
| | 7.52 (m, 2H), 7.40 (d, J = 7.8 Hz, 1H), 7.37 (s, 1H), 4.78-4.49 (m, 1H), 4.11- |
| | 3.83 (m, 2H), 3.86-3.78 (m, 1H), 3.54-3.43 (m, 1H), 3.31-3.23 (m, 2H), |
| | 3.22-3.10 (m, 2H), 2.51 (s, 3H), 2.32-2.11 (m, 2H), 2.03-1.95 (m, 1H), |
| | 1.95-1.85 (m, 1H). 1 NH and 1 OH not observed. |
| 399 | MS m/z 404.3 [M + H]⁺. ¹HNMR (500 MHz, Methanol-d₄) δ 8.75 (s, 1H), 7.66- |
| | 7.56 (m, 2H), 7.44 (s, 1H), 7.36 (s, 1H), 4.35-4.18 (m, 1H), 2.73-2.59 (m, |
| | 2H), 2.28 (t, J = 9.8 Hz, 2H), 2.05-1.89 (m, 1H), 1.44 (s, 3H), 0.93-0.83 (m, |
| | 2H), 0.73-0.71 (m, 2H), NH and OH peak not observed. |
| 400 | MS m/z 462.3 [M + H]⁺. ¹HNMR (500 MHz, Methanol-d₄) δ 8.88 (s, 1H), 7.94 |
| | (s, 1H), 7.89 (d, J = 8.3 Hz, 1H), 7.85 (d, J = 8.1 Hz, 1H), 7.60 (s, 1H), 4.43- |
| | 4.22 (m, 2H), 4.09-3.79 (m, 4H), 3.63-3.47 (m, 1H), 3.40-3.35 (m, 1H), |
| | 2.30-2.03 (m, 1H), 1.96-1.64 (m, 1H). 1 NH and 1 OH not observed. |
| 401 | MS m/z 460.1 [M + H]⁺. ¹HNMR (400 MHz, Methanol-d₄) δ 8.94 (s, 1H), 7.92 |
| | (s, 1H), 7.85 (dd, J = 8.2 Hz, 2H), 7.49 (s, 1H), 4.33 (s, 1H), 4.31-4.25 (m, |
| | 1H), 4.00-3.89 (m, 2H), 2.06-1.91 (m, 2H), 1.89-1.81 (m, 2H), 1.71-1.68 |
| | (m, 2H), 1.51-1.49 (m, 2H). NH and OH peak not observed. |
| 402 | MS m/z 408.3 [M + H]⁺. ¹HNMR (400 MHz, Methanol-d₄) δ 8.82-8.71 (m, |
| | 1H), 7.53-7.46 (m, 1H), 7.46-7.39 (m, 2H), 7.39-7.33 (m, 1H), 4.43-4.24 |
| | (m, 1H), 4.10-4.01 (m, 1H), 4.01-3.93 (m, 1H), 3.83-3.68 (m, 3H), 3.61- |
| | 3.50 (m, 1H), 3.28-3.22 (m, 1H), 2.45 (s, 3H), 2.34-2.20 (m, 1H), 1.86- |
| | 1.67 (m, 1H). NH and OH peak not observed. |

### Example 3

### Preparation of Compound 3

### (1s,3s)-1-Methyl-3-((2-methyl-4-(2-(trifluoromethoxy)-4-(trifluoromethyl)phenyl)pyrazolo[1,5-d][1,2,4]triazin-7-yl)amino)cyclobutan-1-ol

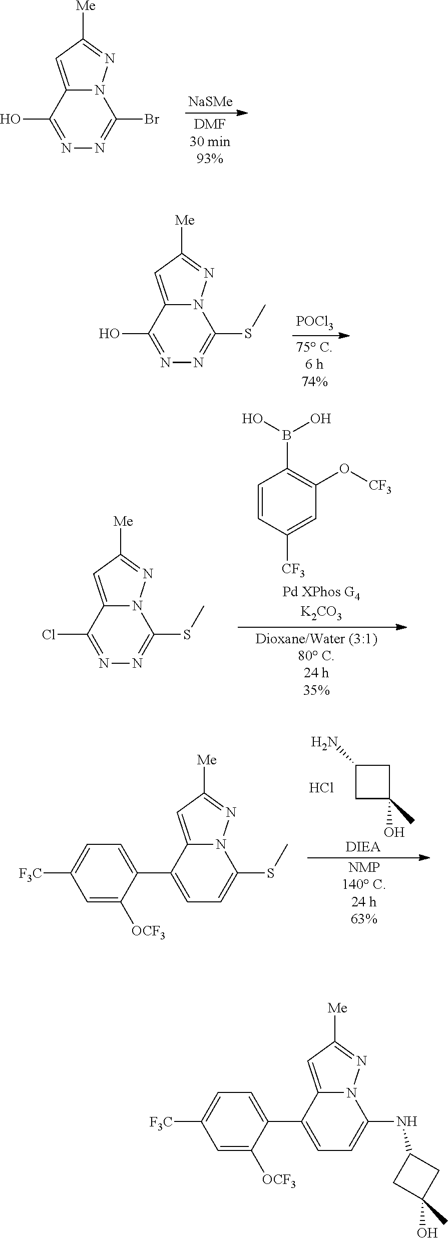

### Step 1. 2-Methyl-7-(methylthio)pyrazolo[1,5-d][1,2,4]triazin-4-ol

A cold solution of 7-bromo-2-methyl-pyrazolo[1,5-d][1,2,4]triazin-4-ol (Intermediate 1f, 600.0 mg, 2.620 mmol) in dry DMF (2.6 mL) at 0° C. was treated with sodium thiomethoxide (386.6 mg, 5.239 mmol). The mixture was then stirred at room temperature for 30 minutes. Upon completion, the DMF was evaporated, and the resulting oily residue was precipitated with water. The mixture was then cooled at 0° C. and the precipitate was filtered while cold. The collected precipitate was washed with water and dried overnight to afford 2-methyl-7-(methylthio)pyrazolo[1,5-d][1,2,4]triazin-4-ol (476.0 mg, 93% yield) as white solid. MS m z 197.1 [M+H]⁺.

### Step 2. 4-Chloro-2-methyl-7-(methylthio)pyrazolo[1,5-d][1,2,4]triazine

A mixture of 2-methyl-7-(methylthio)pyrazolo[1,5-d][1,2,4]triazin-4-ol (476.0 mg, 2.426 mmol) and phosphoryl oxychloride (7.44 g, 4.51 mL, 48.51 mmol) was heated at 75° C. for 6 hours, upon which the solution became clear. The excess POCl₃ was then evaporated, and the resulting residue was precipitated with water. The pH of the aqueous solution was adjusted to neutral (pH˜7) using saturated NaHCO₃ solution. The precipitate was filtered, washed with water, and dried overnight to yield 4-chloro-2-methyl-7-(methylthio)pyrazolo[1,5-d][1,2,4]triazine (383 mg, 74% yield) as white solid. MS m/z 215.1 [M+H]⁺.

### Step 3. 2-Methyl-7-(methylthio)-4-(2-(trifluoromethoxy)-4-(trifluoromethyl)phenyl)-pyrazolo[1,5-d][1,2,4]triazine

A mixture of 4-chloro-2-methyl-7-(methylthio)pyrazolo[1,5-d][1,2,4]triazine (38.8 mg, 0.181 mmol), [2-(trifluoromethoxy)-4-(trifluoromethyl)phenyl]boronic acid (64.4 mg, 0.235 mmol), XPhos Pd G4 (16.4 mg, 0.0181 mmol) and potassium carbonate (74.9 mg, 0.542 mmol) was dissolved in dioxane (2.1 mL) and water (0.4 mL). The reaction vessel was vacuumed and backflushed with nitrogen (3×), and the mixture was stirred at 80° C. under nitrogen atmosphere for 4 hours. Upon completion, the mixture was diluted with EtOAc, washed with water and brine, dried over Na₂SO₄ and evaporated in vacuo. The resulting crude product was purified by flash column chromatography eluting with 0-80% EtOAc in hexane to afford 2-methyl-7-(methylthio)-4-(2-(trifluoromethoxy)-4-(trifluoromethyl)phenyl)-pyrazolo[1,5-d][1,2,4]triazine (25.5 mg, 35% yield) as brown solid. MS m/z 409.1 [M+H]⁺.

### Step 4. (1s,3s)-1-Methyl-3-((2-methyl-4-(2-(trifluoromethoxy)-4-(trifluoromethyl)phenyl)pyrazolo[1,5-d][1,2,4]triazin-7-yl)amino)cyclobutan-1-ol

To a solution of 2-ethyl-7-(methylthio)-4-(2-(trifluoromethoxy)-4-(trifluoromethyl)phenyl)pyrazolo[1,5-d][1,2,4]triazine (25.5 mg, 0.0625 mmol) in NMP (0.50 mL) was added (1s,3s)-3-amino-1-methylcyclobutan-1-ol hydrochloride (25.8 mg, 0.187 mmol) and N,N-diisopropylethylamine (48.4 mg, 65.4 μL, 0.375 mmol). The mixture was heated under nitrogen atmosphere at 130° C. for 24 hours, upon which partial conversion was achieved. Additional (1s,3s)-3-amino-1-methylcyclobutan-1-ol hydrochloride (25.8 mg, 0.187 mmol) was then added and the reaction was stirred and heated further at 140° C. for 24 hours. Upon full conversion to product, the solution was diluted with small amount of water (0.50 mL). The black solution was purified by reverse phase chromatography (0.1% formic acid in MeCN:0.1% formic acid in H₂O, 5 to 100%) to provide (1s,3s)-1-methyl-3-((2-methyl-4-(2-(trifluoromethoxy)-4-(trifluoromethyl)phenyl)pyrazolo[1,5-d][1,2,4]triazin-7-yl)amino)cyclobutan-1-ol (18.2 mg, 63% yield) as white solid. MS m/z 462.1 [M+H]⁺; ¹H NMR (500 MHz, Methanol-d4) δ 7.95 (d, J=8.1 Hz, 1H), 7.89 (d, J=8.1 Hz, 1H), 7.81 (s, 1H), 6.51 (s, 1H), 4.39-4.31 (m, 1H), 2.71-2.61 (m, 2H), 2.54 (s, 3H), 2.36-2.26 (m, 2H), 1.44 (s, 3H).

The compounds below were prepared according to the procedure of Example 3 by substituting the appropriate starting materials, reagents and reaction conditions.

| Compound# | Spectral Data |
|---|---|
| 356 | MS m/z 489.2 [M + H]⁺. ¹HNMR (400 MHz, Methanol-d₄) δ 7.89-7.80 (m, |
| | 1H), 7.77-7.70 (m, 1H), 7.67 (s, 1H), 6.94 (t, J = 73.0 Hz, 1H), 6.51 (s, 1H), |
| | 5.04 (d, J = 47.9 Hz, 1H), 4.83-4.77 (m, 1H), 3.23-3.07 (m, 1H), 2.80-2.59 |
| | (m, 3H), 2.57-2.47 (m, 4H), 2.45-2.31 (m, 1H), 2.20-1.97 (m, 1H), 1.20 (t, |
| | J = 7.1 Hz, 3H). 2 H not observed (NH and a CH obscured by the signal of |
| | Methanol-d₄). |
| 357 | MS m/z 499.0, 501.0 [M + H]⁺. ¹HNMR (400 MHz, Methanol-d₄) δ 8.55 (s, 1H, |
| | formate CH), 7.82-7.69 (m, 2H), 7.64 (d, J = 8.3 Hz, 1H), 6.49 (s, 1H), 4.57- |
| | 4.44 (m, 1H), 3.23-3.15 (m, 1H), 2.92-2.76 (m, 1H), 2.63 (q, J = 7.5 Hz, |
| | 2H), 2.58-2.34 (m, 5H), 2.15-2.01 (m, 1H), 1.97-1.84 (m, 1H), 1.84-1.67 |
| | (m, 2H), 1.18 (t, J = 7.4 Hz, 3H). NH not observed. |
| 358 | MS m/z 423.1, 425.0 [M + H]⁺. ¹HNMR (400 MHz, Methanol-d₄) δ 8.51 (s, 1H, |
| | formic acid), 7.61 (d, J = 9.13 Hz, 1 H), 7.47-7.42 (m, 2H), 6.88 (t, J = 73.54 |
| | Hz, 1H), 6.48 (s, 1H), 4.54-4.46 (m, 1H), 3.28-3.20 (m, 1H), 2.97-2.82 (m, |
| | 1H), 2.71-2.61 (m, 1H), 2.60-2.56 (m, 1 H), 2.53 (s, 3H), 2.52 (s, 3 H), 2.15- |
| | 2.02 (m, 1H), 2.02-1.89 (m, 1H), 1.89-1.67 (m, 2H). 1H not observed |
| | (NH). |
| 359 | MS m/z 441.5 [M + H]⁺. ¹HNMR (400 MHz, Methanol-d₄) δ 8.48 (s, 1H, formic |
| | acid), 8.12 (s, 1H), 8.01 (d, J = 7.63 Hz, 1 H), 7.94 (d, J = 8.88 Hz, 1 H), 7.17 |
| | (t, J = 54.41 Hz, 1 H), 6.58 (s, 1H), 4.59-4.47 (m, 1H), 3.42-3.34 (m, 1H), |
| | 3.08-2.92 (m, 1H), 2.82-2.64 (m, 2H), 2.61 (s, 3H), 2.54 (s, 3H), 2.19-2.05 |
| | (m, 1H), 2.05-1.94 (m, 1H), 1.91-1.74 (m, 2H). 1H not observed (NH). |
| 360 | MS m/z 429.2 [M + H]⁺. ¹HNMR (400 MHz, Methanol-d₄) δ 8.45 (s, 1H, formic |
| | acid), 7.47 (d, J = 8.63 Hz, 1H), 7.12 (d, J = 8.63 Hz, 1H), 7.07 (s, 1H), 6.79 (t, |
| | J = 75.67 Hz, 1H), 6.47 (s, 1H), 4.59-4.46 (m, 1H), 3.58-3.43 (m, 1H), 3.22- |
| | 3.06 (m, 1H), 2.99-2.77 (m, 2H), 2.72 (s, 3H), 2.52 (s, 3H), 2.24-2.11 (m, |
| | 1H), 2.11-1.97 (m, 2H), 1.97-1.70 (m, 2H), 1.16-1.03 (m, 2H), 0.86-0.74 |
| | (m, 2H). 1H not observed (NH). |
| 361 | MS m/z 403.3 [M + H]⁺. ¹HNMR (400 MHz, Methanol-d₄) δ 8.42 (s, 1H, formic |
| | acid), 7.48 (d, J = 7.8 Hz, 1H), 7.24 (d, J = 8.0 Hz, 1H), 7.21 (s, 1H), 6.80 (t, |
| | J = 74.29 Hz, 1H), 6.48 (s, 1H), 4.62-4.50 (m, 1H), 3.68-3.55 (m, 1H), 3.30- |
| | 3.22 (m, 1H), 3.10-2.85 (m, 2H), 2.81 (s, 3H), 2.52 (s, 3H), 2.47 (s, 3H), 2.24- |
| | 2.05 (m, 2H), 1.97-1.81 (m, 2H). 1H not observed (NH). |
| 362 | MS m/z 407.1, 409.0 [M + H]⁺. ¹HNMR (400 MHz, Methanol-d₄) δ 7.83 (s, 1H), |
| | 7.74-7.66 (m, 2H), 7.08 (t, J = 55.7 Hz, 1H), 6.53 (s, 1H), 4.49-4.41 (m, 1H), |
| | 3.09-2.88 (m, 1H), 2.73-2.57 (m, 1H), 2.53 (s, 3H), 2.47-2.27 (m, 5H), |
| | 2.09-1.94 (m, 1H), 1.94-1.81 (m, 1H), 1.80-1.63 (m, 2H). 1H not observed |
| | (NH). |
| 363 | MS m/z 413.4 [M + H]⁺. ¹HNMR (400 MHz, Methanol-d₄) δ 8.48 (br s, 1H, |
| | formic acid), 7.57-7.52 (m, 2H), 7.37 (d, J = 8.4 Hz, 1H), 7.00 (t, J = 55.66 |
| | Hz, 1H), 6.51 (s, 1H), 4.56-4.44 (m, 1H), 3.42-3.34 (m, 1H), 3.10-2.95 (m, |
| | 1H), 2.82-2.65 (m, 2H), 2.63 (s, 3H), 2.52 (s, 3H), 2.16-1.94 (m, 3H), 1.92- |
| | 1.70 (m, 2H), 1.14-1.07 (m, 2H), 0.87-0.77 (m, 2H). 1H not observed (NH). |
| 364 | MS m/z 387.3 [M + H]⁺. ¹HNMR (400 MHz, Methanol-d₄) δ 8.53 (br s, 1H, |
| | formic acid), 7.67 (s, 1 H), 7.56 (d, J = 8.25 Hz, 1 H), 7.49 (d, J = 8.63 Hz, 1 |
| | H), 7.01 (d, J = 55.7 Hz, 1H), 6.48 (s, 1H), 4.53-4.40 (m, 1H), 3.18-3.01 (m, |
| | 1H), 2.83-2.66 (m, 1H), 2.56-2.39 (m, 11H), 2.09-1.98 (m, 1H), 1.97- |
| | 1.84 (m, 1H), 1.83-1.64 (m, 2H). 1H not observed (NH). |
| 365 | MS m/z 489.3 [M + H]⁺. ¹HNMR (400 MHz, Methanol-d₄) δ 7.95 (d, J = 8.1 Hz, |
| | 1H), 7.90 (d, J = 8.9 Hz, 1H), 7.82 (s, 1H), 6.51 (s, 1H), 4.56-4.45 (m, 1H), |
| | 3.12-3.03 (m, 1H), 2.80-2.66 (m, 1H), 2.62-2.48 (m, 5H), 2.48-2.30 (m, |
| | 2H), 2.12-1.99 (m, 1H), 1.93-1.82 (m, 1H), 1.82-1.65 (m, 2H), 1.15 (t, J = |
| | 7.3 Hz, 3H). NH not observed. |
| 366 | MS m/z 467.3 [M + H]⁺. ¹HNMR (400 MHz, Methanol-d₄) δ 8.54 (s, 1H, formate |
| | CH), 7.61 (d, J = 8.1 Hz, 1H), 7.42 (dd, J = 8.1, 1.9 Hz, 1H), 7.33 (s, 1H), 6.49 |
| | (s, 1H), 4.57-4.47 (m, 1H), 3.00-2.84 (m, 1H), 2.79-2.65 (m, 2H), 2.65- |
| | 2.40 (m, 7H), 2.16-2.04 (m, 1H), 2.02-1.89 (m, 1H), 1.87-1.72 (m, 2H), |
| | 1.20 (t, J = 7.3 Hz, 3H). 2 CH signals overlap with Methanol-d₄ peak; NH not |
| | observed. |
| 367 | MS m/z 397.4 [M + H]⁺. ¹HNMR (400 MHz, Methanol-d₄) δ 7.32 (s, 1H), 7.30 |
| | (d, J = 7.8 Hz, 1H), 7.13 (d, J = 7.8 Hz, 1H), 6.34 (s, 1H), 4.52-4.40 (m, 1H), |
| | 3.17-3.03 (m, 1H), 2.77-2.65 (m, 1H), 2.59-2.48 (m, 5H), 2.48-2.31 (m, |
| | 8H), 2.08-1.97 (m, 1H), 1.91-1.81 (m, 1H), 1.78-1.66 (m, 2H), 1.14 (t, J = |
| | 7.3 Hz, 3H). NH not observed. |
| 368 | MS m/z 429.3 [M + H]⁺. ¹HNMR (400 MHz, Methanol-d₄) δ 8.06 (s, 1H), 7.70 |
| | (d, J = 8.0 Hz, 1H), 7.56 (d, J = 7.9 Hz, 1H), 6.36 (s, 1H), 4.64-4.52 (m, 1H), |
| | 3.99-3.89 (m, 1H), 3.70-3.55 (m, 1H), 3.45-3.34 (m, 1H), 3.29-3.25 (m, |
| | 2H), 3.03-2.85 (m, 2H), 2.56 (s, 3H), 2.50 (s, 3H), 2.37-2.26 (m, 1H), 2.23- |
| | 2.14 (m, 1H), 2.06-1.75 (m, 2H), 1.40 (t, J = 7.3 Hz, 3H). 2 CH signals |
| | overlap with Methanol-d₄ peak; NH not observed. |
| 369 | MS m/z 499.2 [M + H]⁺. ¹HNMR (400 MHz, Methanol-d₄) δ 8.16 (dd, J = 8.1, |
| | 1.7 Hz, 1H), 8.10 (s, 1H), 8.00 (d, J = 8.0 Hz, 1H), 6.58 (s, 1H), 4.69-4.59 (m, |
| | 1H), 4.00-3.92 (m, 1H), 3.69-3.61 (m, 1H), 3.28-3.23 (m, 5H), 2.95 (t, J = |
| | 11.8 Hz, 2H), 2.55 (s, 3H), 2.35-2.28 (m, 1H), 2.24-2.16 (m, 1H), 2.00- |
| | 1.83 (m, 2H), 1.40 (t, J = 7.3 Hz, 3H). NH not observed. |
| 370 | MS m/z 427.4 [M + H]⁺. ¹HNMR (400 MHz, Methanol-d₄) δ 7.19 (d, J = 8.6 Hz, |
| | 1H), 6.93 (d, J = 8.6 Hz, 1H), 6.44 (s, 1H), 4.52-4.41 (m, 1H), 3.92 (s, 3H), |
| | 3.90 (s, 3H), 3.69 (s, 3H), 3.20-3.14 (m, 1H), 2.86-2.70 (m, 1H), 2.66-2.55 |
| | (m, 2H), 2.51 (s, 3H), 2.48-2.32 (m, 2H), 2.12-2.00 (m, 1H), 1.93-1.82 (m, |
| | 1H), 1.82-1.65 (m, 2H), 1.16 (t, J = 7.3 Hz, 3H). NH not observed. |
| 371 | MS m/z 451.3 [M + H]⁺. ¹HNMR (400 MHz, Methanol-d₄) δ 7.61 (d, J = 8.6 Hz, |
| | 1H), 7.12 (dd, J = 8.6, 2.5 Hz, 1H), 7.03 (dq, J = 2.9, 1.5 Hz, 1H), 6.45 (s, 1H), |
| | 4.52-4.41 (m, 1H), 3.91 (s, 3H), 3.18-3.03 (m, 1H), 2.78-2.65 (m, 1H), |
| | 2.60-2.47 (m, 5H), 2.47-2.28 (m, 2H), 2.10-1.99 (m, 1H), 1.93-1.81 (m, |
| | 1H), 1.80-1.66 (m, 2H), 1.14 (t, J = 7.3 Hz, 3H). NH not observed. |
| 372 | MS m/z 448.3 [M + H]⁺. ¹HNMR (400 MHz, DMSO-d₆) δ 8.18 (s, 1H, formate |
| | CH), 8.10 (s, 1H), 8.03-7.91 (m, 2H), 7.85 (d, J = 7.9 Hz, 1H), 7.63-7.54 (m, |
| | 1H), 7.47 (s, 1H), 6.51 (s, 1H), 4.43-4.25 (m, 1H), 2.88 (d, J = 8.5 Hz, 1H), |
| | 2.66-2.58 (m, 1H), 2.46 (s, 3H), 2.40 (q, J = 7.1 Hz, 2H), 2.31-2.21 (m, 1H), |
| | 2.20-2.05 (m, 1H), 1.91-1.78 (m, 1H), 1.78-1.63 (m, 2H), 1.63-1.46 (m, |
| | 1H), 1.02 (t, J = 7.1 Hz, 3H). |
| 373 | MS m/z 435.3 [M + H]⁺. ¹HNMR (400 MHz, Methanol-d₄) δ 7.51 (d, J = 8.4 Hz, |
| | 1H), 7.38 (d, J = 2.6 Hz, 1H), 7.32 (dd, J = 8.6, 2.7 Hz, 1H), 6.29 (s, 1H), 4.51- |
| | 4.43 (m, 1H), 3.94 (s, 3H), 3.15-3.05 (m, 1H), 2.79-2.66 (m, 1H), 2.61- |
| | 2.48 (m, 5H), 2.48-2.32 (m, 2H), 2.10-1.99 (m, 1H), 1.92-1.81 (m, 1H), |
| | 1.78-1.67 (m, 2H), 1.15 (t, J = 7.3 Hz, 3H). NH not observed. |
| 374 | MS m/z 401.3, 403.3 [M + H]⁺. ¹HNMR (400 MHz, Methanol-d₄) δ 7.46 (d, J = |
| | 8.6 Hz, 1H), 7.16 (d, J = 2.5 Hz, 1H), 7.05 (dd, J = 8.6, 2.6 Hz, 1H), 6.39 (s, |
| | 1H), 4.53-4.43 (m, 1H), 3.89 (s, 3H), 3.23-3.15 (m, 1H), 2.88-2.75 (m, |
| | 1H), 2.68-2.56 (m, 2H), 2.54-2.39 (m, 5H), 2.10-2.01 (m, 1H), 1.95-1.85 |
| | (m, 1H), 1.83-1.68 (m, 2H), 1.17 (t, J = 7.3 Hz, 3H). NH not observed. |
| 375 | MS m/z 439.2, 441.2 [M + H]⁺. ¹HNMR (500 MHz, Methanol-d₄) δ 7.95 (d, J = |
| | 1.8 Hz, 1H), 7.82 (dd, J = 8.2, 1.9 Hz, 1H), 7.78 (d, J = 7.9 Hz, 1H), 6.44 (s, |
| | 1H), 4.55-4.48 (m, 1H), 3.25-3.18 (m, 1H), 2.90-2.79 (m, 1H), 2.71-2.59 |
| | (m, 2H), 2.59-2.36 (m, 5H), 2.14-2.04 (m, 1H), 1.96-1.87 (m, 1H), 1.84- |
| | 1.71 (m, 2H), 1.18 (t, J = 7.2 Hz, 3H). NH not observed. |
| 376 | MS m/z 405.2, 407.2, 409.2 [M + H]⁺. ¹HNMR (500 MHz, Methanol-d₄) δ 7.69 |
| | (d, J = 2.0 Hz, 1H), 7.55 (d, J = 8.2 Hz, 1H), 7.52 (dd, J = 8.2, 2.0 Hz, 1H), 6.42 |
| | (s, 1H), 4.57-4.46 (m, 1H), 3.29-3.23 (m, 1H), 2.99-2.81 (m, 1H), 2.77- |
| | 2.66 (m, 2H), 2.61-2.46 (m, 5H), 2.14-2.04 (m, 1H), 1.99-1.89 (m, 1H), |
| | 1.85-1.72 (m, 2H), 1.20 (t, J = 7.2 Hz, 3H). NH not observed. |
| 377 | MS m/z 371.3, 373.3 [M + H]⁺. ¹HNMR (500 MHz, Methanol-d₄) δ 7.99-7.86 |
| | (m, 2H), 7.62-7.52 (m, 2H), 6.87 (s, 1H), 4.56-4.47 (m, 1H), 3.42-3.34 (m, |
| | 1H), 3.10-3.00 (m, 1H), 2.89-2.75 (m, 2H), 2.75-2.60 (m, 2H), 2.56 (s, |
| | 3H), 2.16-2.08 (m, 1H), 2.04-1.95 (m, 1H), 1.88-1.75 (m, 2H), 1.24 (t, J = |
| | 7.2 Hz, 3H). NH not observed. |
| 378 | MS m/z 428.3 [M + H]⁺. ¹HNMR (500 MHz, Methanol-d₄) δ 7.82 (d, J = 8.4 Hz, |
| | 1H), 7.80-7.70 (m, 2H), 6.93 (t, J = 72.8 Hz, 1H), 6.49 (s, 1H), 4.52-4.46 (m, |
| | 1H), 3.15-3.08 (m, 1H), 2.76-2.69 (m, 1H), 2.62-2.31 (m, 7H), 2.09-2.01 |
| | (m, 1H), 1.92-1.83 (m, 1H), 1.78-1.69 (m, 2H), 1.15 (d, J = 7.3 Hz, 3H). NH |
| | not observed. |
| 379 | MS m/z 407.3 [M + H]⁺. ¹HNMR (500 MHz, Methanol-d₄) δ 7.30 (d, J = 8.5 Hz, |
| | 1H), 6.86 (dd, J = 8.4, 2.6 Hz, 1H), 6.61 (d, J = 2.7 Hz, 1H), 6.39 (s, 1H), 4.49- |
| | 4.42 (m, 1H), 3.83 (s, 3H), 3.15-3.06 (m, 1H), 2.78-2.67 (m, 1H), 2.54 (q, J = |
| | 7.9 Hz, 2H), 2.50 (s, 3H), 2.45-2.31 (m, 2H), 2.08-1.99 (m, 1H), 1.92- |
| | 1.81 (m, 2H), 1.79-1.66 (m, 2H), 1.15 (t, J = 7.2 Hz, 3H), 0.77 (dt, J = 6.6, 1.9 |
| | Hz, 2H), 0.67-0.61 (m, 2H). NH not observed. |
| 380 | MS m/z 444.2 [M + H]⁺. ¹HNMR (500 MHz, Methanol-d₄) δ 7.84 (d, J = 7.9 Hz, |
| | 1H), 7.74 (d, J = 8.1 Hz, 1H), 7.66 (s, 1H), 6.93 (t, J = 72.7 Hz, 1H), 6.48 (s, |
| | 1H), 4.34 (p, J = 7.9 Hz, 1H), 2.70-2.62 (m, 2H), 2.53 (s, 3H), 2.35-2.26 (m, |
| | 2H), 1.43 (s, 3H). NH and OH not observed. |
| 381 | MS m/z 458.2 [M + H]⁺. ¹HNMR (500 MHz, Methanol-d₄) δ 7.85 (d, J = 7.8 Hz, |
| | 1H), 7.74 (d, J = 8.1 Hz, 1H), 7.66 (s, 1H), 6.93 (d, J = 72.6 Hz, 1H), 6.48 (s, |
| | 1H), 4.10 (td, J = 10.2, 4.3 Hz, 1H), 3.73-3.64 (m, 1H), 2.52 (s, 3H), 2.29- |
| | 2.21 (m, 1H), 2.14-2.07 (m, 1H), 1.85-1.76 (m, 2H), 1.59-1.37 (m, 4H). |
| | NH and OH not observed. |
| 382 | MS m/z 476.2 [M + H]⁺. ¹HNMR (500 MHz, Methanol-d₄) δ 7.95 (d, J = 8.1 Hz, |
| | 1H), 7.89 (d, J = 8.1 Hz, 1H), 7.81 (s, 1H), 6.50 (s, 1H), 4.12 (td, J = 10.2, 4.2 |
| | Hz, 1H), 3.73-3.64 (m, 1H), 2.53 (s, 3H), 2.29-2.21 (m, 1H), 2.15-2.07 (m, |
| | 1H), 1.85-1.76 (m, 2H), 1.58-1.37 (m, 4H). NH and OH not observed. |
| 383 | MS m/z 480.3 [M + H]; ¹HNMR (500 MHz, Methanol-d₄) δ 7.82 (d, J = 7.9 Hz, |
| | 1H), 7.72 (d, J = 8.1 Hz, 1H), 7.65 (s, 1H), 7.16 (t, J = 7.9 Hz, 1H), 6.97 (d, J = |
| | 7.5 Hz, 1H), 6.93 (s, 1H), 6.91 (t, JCF = 73.6 Hz, 1H), 6.68 (dd, J = 8.0, 2.7 Hz, |
| | 1H), 6.49 (s, 1H), 5.47 (q, J = 7.2 Hz, 1H), 2.54 (s, 3H), 1.70 (d, J = 7.0 Hz, |
| | 3H). NH and OH not observed. |
| 384 | MS m/z 438.3 [M + H]⁺. ¹HNMR (500 MHz, Methanol-d₄) δ 7.50 (d, J = 8.4 Hz, |
| | 1H), 7.12 (d, J = 3.1 Hz, 1H), 7.09 (dd, J = 8.5, 2.8 Hz, 1H), 6.44 (s, 1H), 4.27 |
| | (td, J = 9.9, 4.6 Hz, 1H), 4.03 (dd, J = 11.1, 5.0 Hz, 1H), 3.97 (dd, J = 11.5, 4.0 |
| | Hz, 1H), 3.89 (s, 3H), 3.85-3.71 (m, 3H), 3.56 (td, J = 12.0, 2.2 Hz, 1H), 3.29- |
| | 3.23 (m, 1H), 2.52 (s, 3H), 2.29-2.22 (m, 1H), 1.89-1.78 (m, 1H). NH and |
| | OH not observed. |
| 385 | MS m/z 436.3 [M + H]⁺. ¹HNMR (500 MHz, Methanol-d₄) δ 7.50 (d, J = 8.5 Hz, |
| | 1H), 7.11 (s, 1H), 7.08 (dd, J = 8.5, 2.7 Hz, 1H), 6.43 (s, 1H), 4.10-4.03 (m, |
| | 1H), 3.89 (s, 3H), 3.84-3.73 (m, 2H), 3.73-3.63 (m, 1H), 2.52 (s, 3H), 2.32- |
| | 2.21 (m, 1H), 2.17-2.05 (m, 1H), 1.88-1.74 (m, 2H), 1.58-1.36 (m, 4H). |
| | NH and OH not observed. |
| 386 | MS m/z 421.3 [M + H]⁺. ¹HNMR (500 MHz, Methanol-d₄) δ 7.50 (d, J = 8.5 Hz, |
| | 1H), 7.12 (d, J = 2.9 Hz, 1H), 7.09 (dd, J = 8.4, 2.7 Hz, 1H), 6.45 (s, 1H), 5.06- |
| | 4.97 (m, 1H), 3.89 (s, 3H), 3.76 (q, J = 11.1 Hz, 2H), 3.54-3.43 (m, 2H), 2.79- |
| | 2.69 (m, 1H), 2.52 (s, 3H), 2.44-2.33 (m, 1H). NH not observed. |
| 387 | MS m/z 422.3 [M + H]⁺. ¹HNMR (500 MHz, Methanol-d₄) δ 7.50 (d, J = 8.5 Hz, |
| | 1H), 7.11 (d, J = 2.9 Hz, 1H), 7.08 (dd, J = 8.5, 2.5 Hz, 1H), 6.43 (s, 1H), 4.32 |
| | (p, J = 8.2 Hz, 1H), 3.89 (s, 3H), 3.79 (q, J = 11.1 Hz, 2H), 2.73-2.59 (m, 2H), |
| | 2.52 (s, 3H), 2.35-2.23 (m, 2H), 1.43 (s, 3H). |
| 388 | MS m/z 460.1 [M + H]⁺. ¹HNMR (400 MHz, DMSO-d₆) δ 8.30 (s, 1H), 8.00 (s, |
| | 1H), 7.92 (d, J = 8.2 Hz, 1H), 7.84 (d, J = 8.1 Hz, 1H), 6.55 (s, 1H), 4.98 (s, |
| | 1H), 4.35-4.24 (m, 1H), 4.15-4.06 (m, 2H), 2.47 (s, 3H), 2.46-2.40 (m, |
| | 2H), 2.35-2.27 (m, 2H), 1.31 (s, 3H). |
| 389 | MS m/z 474.1 [M + H]⁺. ¹HNMR (400 MHz, Methanol-d₄) δ 7.90 (s, 1H), 7.84 |
| | (d, J = 8.4 Hz, 1H), 7.80 (d, J = 8.4 Hz, 1H), 6.46 (s, 1H), 4.14-4.06 (m, 1H), |
| | 4.00-3.89 (m, 2H), 3.73-3.64 (m, 1H), 2.52 (s, 3H), 2.26-2.23 (m, 1H), |
| | 2.12-2.09 (m, 1H), 1.83-1.80 (m, 2H), 1.55-1.38 (m, 4H). NH and OH not |
| | observed. |
| 390 | MS m/z 406.3 [M + H]⁺. ¹HNMR (500 MHz, Methanol-d₄) δ 7.45 (d, J = 7.8 Hz, |
| | 1H), 7.39 (s, 1H), 7.35 (br d, J = 7.8 Hz, 1H), 6.43 (s, 1H), 4.32 (quint, J = 7.9 |
| | Hz, 1H), 3.77 (q, J = 11.0 Hz, 2H), 2.69-2.62 (m, 2H), 2.52 (s, 3H), 2.45 (s, |
| | 3H), 2.30 (t, J = 9.8 Hz, 2H), 1.43 (s, 3H). 2H not observed (NH and OH). |
| 391 | MS m/z 424.3 [M + H]⁺. ¹HNMR (400 MHz, DMSO-d₆) δ 8.16 (d, J = 6.9 Hz, |
| | 1H), 7.64 (d, J = 8.6 Hz, 1H), 7.16 (dd, J = 8.7, 2.5 Hz, 1H), 7.09 (s, 1H), 6.45 |
| | (s, 1H), 4.97 (s, 1H), 4.25 (dd, J = 15.1, 7.5 Hz, 1H), 3.89 (s, 3H), 2.47 (s, 3H), |
| | 2.46-2.39 (m, 2H), 2.33-2.26 (m, 2H), 1.31 (s, 3H). |
| 392 | MS m/z 438.3 [M + H]⁺. ¹HNMR (400 MHz, DMSO-d₆) δ 7.64 (d, J = 8.6 Hz, |
| | 1H), 7.60 (d, J = 8.1 Hz, 1H), 7.17 (dd, J = 8.7, 2.5 Hz, 1H), 7.09 (s, 1H), 6.44 |
| | (s, 1H), 4.76 (d, J = 5.4 Hz, 1H), 4.09-3.92 (m, 1H), 3.89 (s, 3H), 3.71-3.56 |
| | (m, 1H), 2.47 (s, 3H), 2.09-2.01 (m, 1H), 1.99-1.91 (m, 1H), 1.72-1.64 (m, |
| | 2H), 1.39-1.18 (m, 4H). |

### Example 4

### Preparation of Compound 4

### (1s,3s)-3-((4-(2,4-Bis(trifluoromethyl)phenyl)pyrazolo[1,5-d][1,2,4]triazin-7-yl)amino)-1-methylcyclobutan-1-ol

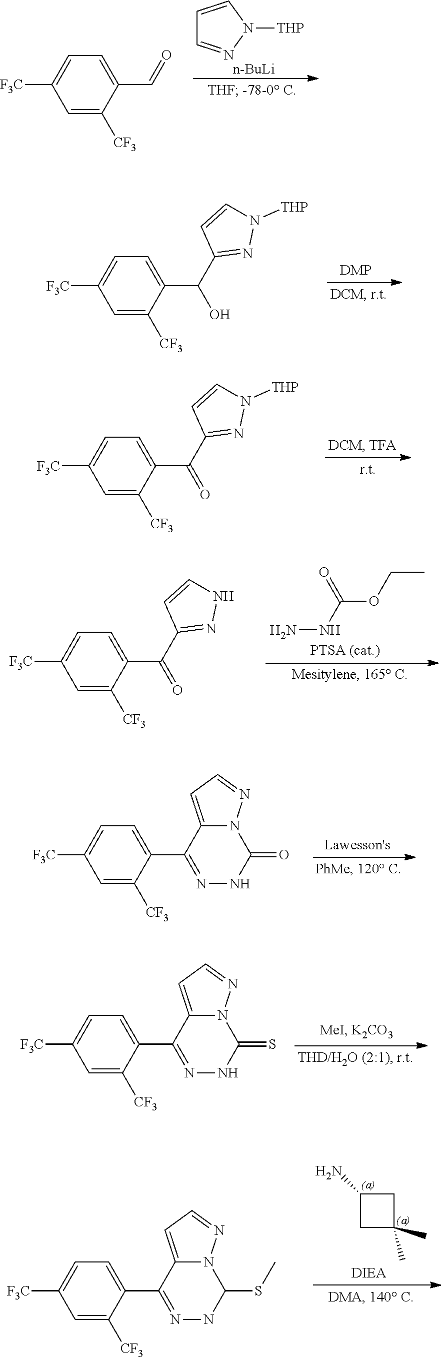

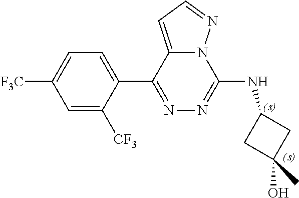 [SMILES: C[C@]1(O)C[C@@H](Nc2nnc(-c3ccc(C(F)(F)F)cc3C(F)(F)F)c3ccnn23)C1]

### Step 1. (2,4-Bis(trifluoromethyl)phenyl)(1-(tetrahydro-2H-pyran-2-yl)-1H-pyrazol-3-yl) methanol

To a solution of 1-(tetrahydro-2H-pyran-2-yl)-1H-pyrazole (5.3 g, 35.0 mmol, 1.0 eq.) in tetrahydrofuran (170 mL) was added n-butyl lithium (16.8 mL, 1.2 eq., 2.5 M in hexane) dropwise at −78° C. The reaction mixture was stirred at −78° C. for 30 min, then 2,4-bis(trifluoromethyl)benzaldehyde (11.02 g, 1.3 eq.) was added. The mixture was then warmed to room temperature and stirred for 2 hours before quenching with water (200 mL). The resulting mixture was extracted with ethyl acetate (3×100 mL). The organic phase was dried with sodium sulfate, filtered and concentrated under reduced pressure to afford crude (2,4-bis(trifluoromethyl)phenyl)(1-(tetrahydro-2H-pyran-2-yl)-1H-pyrazol-3-yl) methanol (15.0 g, crude) as a dark yellow oil, which was used in the next step without further purification.

### Step 2. (2,4-Bis(trifluoromethyl)phenyl)(1-(tetrahydro-2H-pyran-2-yl)-1H-pyrazol-3-yl) methanone

To a solution of (2,4-bis(trifluoromethyl)phenyl)(1-(tetrahydro-2H-pyran-2-yl)-1H-pyrazol-3-yl) methanol (15.0 g, crude) in dichloromethane (170 mL) at 0° C. was added Dess-Martin periodinane (19.3 g, mmol, 1.3 eq.) in portions. The reaction mixture was stirred at room temperature for 2 hours then washed with sat. Na₂S₂O₃ solution (2×100 mL), sat. NaHCO₃ solution (100 mL) and brine (100 mL). The organic phase was dried with sodium sulfate, filtered and concentrated under reduced pressure to afford (2,4-bis(trifluoromethyl)phenyl)(1-(tetrahydro-2H-pyran-2-yl)-1H-pyrazol-3-yl) methanone (15 g, crude) as a dark yellow oil, which was used in the next step without further purification. ¹H NMR (400 MHz, DMSO-d₆) δ 8.26 (s, 1H), 8.23 (d, J=8.4 Hz, 1H), 7.93 (d, J=8.0 Hz, 1H), 7.68 (d, J=2.0 Hz, 1H), 6.66 (d, J=2.0 Hz, 1H), 6.17 (dd, J=10.0, 2.0 Hz, 1H), 3.95 (d, J=11.6 Hz, 1H), 3.71-3.57 (m, 1H), 2.38-2.27 (m, 1H), 2.07-1.93 (m, 2H), 1.78-1.64 (m, 1H), 1.61-1.51 (m, 2H).

### Step 3. (2,4-Bis(trifluoromethyl)phenyl)(1H-pyrazol-3-yl)methanone

(2,4-Bis(trifluoromethyl)phenyl)(1-(tetrahydro-2H-pyran-2-yl)-1H-pyrazol-3-yl) methanone (15 g, crude) was dissolved in a solution of dichloromethane (300 mL) and trifluoroacetic acid (30 mL). The resulting mixture was stirred at room temperature for 16 h. Upon completion, the reaction mixture was adjusted to pH˜7 by addition of sat. NaHCO₃ solution. The organic phase was partitioned, dried with sodium sulfate, filtered and concentrated under reduced pressure. The residue was purified by silica gel column chromatography eluting with 0-50% EtOAc in hexanes to afford (2,4-bis(trifluoromethyl)phenyl)(1H-pyrazol-3-yl)methanone (6.50 g, 60.2% yield over 3 steps) as a yellow solid. MS m/z 309.0 [M+H]⁺; ¹H NMR (400 MHz, DMSO-d₆) δ 13.68 (s, 1H), 8.21 (s, 1H), 8.18 (d, J=8.4 Hz, 1H), 7.99 (s, 1H), 7.88 (d, J=8.0 Hz, 1H), 6.98 (s, 1H).

### Step 4. 4-(2,4-Bis(trifluoromethyl)phenyl)pyrazolo[1,5-d][1,2,4]triazin-7(6H)-one

A mixture of (2,4-bis(trifluoromethyl)phenyl)(1H-pyrazol-3-yl)methanone (3.0 g, 9.7 mmol, 1.0 eq.) and ethyl hydrazine carboxylate (1.01 g, 1.0 eq.) in mesitylene (100 mL) was heated at 165° C. for 4 h. Upon completion, the reaction mixture was cooled to room temperature, then directly purified by silica gel column chromatography eluting with 0-40% EtOAc in hexanes to afford 4-(2,4-bis(trifluoromethyl)phenyl)pyrazolo[1,5-d][1,2,4]triazin-7(6H)-one (1.50 g, 44.4% yield) as a light yellow solid. MS m/z 349.1 [M+H]⁺; ¹H NMR (400 MHz, DMSO-d₆) δ 13.68 (s, 1H), 8.21 (s, 1H), 8.18 (d, J=8.0 Hz, 1H), 7.99 (dd, J=2.8, 1.6 Hz, 1H), 7.88 (d, J=8.0 Hz, 1H), 6.98 (dd, J=2.4, 2.0 Hz, 1H).

### Step 5. 4-(2,4-Bis(trifluoromethyl)phenyl)pyrazolo[1,5-d][1,2,4]triazine-7(6H)-thione

A mixture of 4-(2,4-bis(trifluoromethyl)phenyl)pyrazolo[1,5-d][1,2,4]triazin-7(6H)-one (1.50 g, 4.31 mmol, 1.0 eq.) and Lawesson's reagent (3.49 g, 2.0 eq.) in toluene (40 mL) was heated at 120° C. for 16 h. Upon completion, the reaction mixture was cooled to room temperature, then filtered through a pad of Celite. The filtrate was washed with water (80 mL). The organic phase was dried with sodium sulfate, filtered and concentrated under reduced pressure to afford 4-(2,4-bis(trifluoromethyl)phenyl)pyrazolo[1,5-d][1,2,4]triazine-7(6H)-thione (1.30 g, crude) as a light yellow solid, which was used in the next step without further purification.

### Step 6. 4-(2,4-Bis(trifluoromethyl)phenyl)-7-(methylthio)pyrazolo[1,5-d][1,2,4]triazine

To a mixture of 4-(2,4-bis(trifluoromethyl)phenyl)pyrazolo[1,5-d][1,2,4]triazine-7(6H)-thione (1.30 g, 1.0 eq.) and K₂CO₃ (1.49 g, 2.5 eq.) in THE (12 mL) and water (6 mL) was added iodomethane (0.4 mL, 1.5 eq.) dropwise. The mixture was stirred at room temperature for 1 hour, then diluted with water (50 mL) and extracted with ethyl acetate (3×50 mL). The organic layer was dried over sodium sulfate, filtered and concentrated under reduced pressure. The crude product was purified by silica gel column chromatography eluting with 0-30% EtOAc in hexanes to afford 4-(2,4-bis(trifluoro-methyl)phenyl)-7-(methylthio)pyrazolo[1,5-d][1,2,4]triazine (1.08 g, 66.2% yield over 2 steps) as a light yellow solid. MS m/z 379.3 [M+H]⁺.

### Step 7. (1s,3s)-3-((4-(2,4-Bis(trifluoromethyl)phenyl)pyrazolo[1,5-d][1,2,4]triazin-7-yl) amino)-1-methylcyclobutan-1-ol

A mixture of 4-(2,4-bis(trifluoro-methyl)phenyl)-7-(methylthio)pyrazolo[1,5-d][1,2,4]triazine (200 mg, 0.53 mmol, 1.0 eq.), (1s,3s)-3-amino-1-methylcyclobutan-1-ol hydrochloride (180.9 mg, 2.5 eq.) and DIEA (1.20 mL, 13.0 eq.) in DMAc (0.75 mL) was heated at 140° C. for 4 h. Upon completion, the reaction mixture was cooled to room temperature and diluted with water (50 mL) and extracted with EtOAc (3×50 mL). The organic phase was washed with brine (100 mL), dried over Na₂SO₄, filtered and concentrated under reduced pressure. The residue was purified by reverse phase chromatography to afford (1s,3s)-3-((4-(2,4-bis(trifluoromethyl)phenyl)pyrazolo[1,5-d][1,2,4]triazin-7-yl)amino)-1-methylcyclobutan-1-ol (68.9 mg, 30.1% yield) as a white solid. MS m/z 432.1 [M+H]⁺. ¹H NMR (400 MHz, Methanol-d₄) δ 8.21-8.18 (m, 2H), 8.14 (d, J=8.4 Hz, 1H), 7.88 (d, J=8.0 Hz, 1H), 6.56 (d, J=2.0 Hz, 1H), 4.36 (quin, J=8.0 Hz, 1H), 2.71-2.62 (m, 2H), 2.37-2.29 (m, 2H), 1.44 (s, 3H).

The compounds below were prepared according to the procedure of Example 4 by substituting the appropriate starting materials, reagents and reaction conditions.

| Compound# | Spectral Data |
|---|---|
| 393 | MS m/z 447.1 [M + H]⁺. ¹HNMR (400 MHz, Methanol-d₄) δ 8.26 (d, J = 2.0 |
| | Hz, 1H), 8.00 (d, J = 7.6 Hz, 1H), 7.93 (d, J = 8.0 Hz, 1H), 7.85 (s, 1H), |
| | 6.76 (d, J = 2.0 Hz, 1H), 5.15-5.06 (m, 1H), 3.56-3.47 (m, 2H), 2.81- |
| | 2.70 (m, 1H), 2.52-2.38 (m, 1H). NH not observed. |
| 394 | MS m/z 446.1 [M + H]⁺. ¹HNMR (400 MHz, Methanol-d₄) δ 8.21-8.17 (m, |
| | 2H), 8.14 (d, J = 8.0 Hz, 1H), 7.88 (d, J = 8.0 Hz, 1H), 6.56 (d, J = 2.0 Hz, |
| | 1H), 4.20-4.07 (m, 1H), 3.75-3.65 (m, 1H), 2.30-2.22 (m, 1H), 2.16- |
| | 2.06 (m, 1H), 1.85-1.76 (m, 2H), 1.58-1.42 (m, 4H). NH and OH not |
| | observed. |
| 395 | MS m/z 468.0 [M + H]⁺. ¹HNMR (400 MHz, Methanol-d₄) δ 8.21 (d, J = 2.4 |
| | Hz, 1H), 8.18 (s, 1H), 8.12 (d, J = 8.0 Hz, 1H), 7.86 (d, J = 8.0 Hz, 1H), |
| | 7.16 (t, J = 8.0 Hz, 1H), 7.01-6.96 (m, 1H), 6.95-6.93 (m, 1H), 6.68 (dd, J = |
| | 8.0, 2.0 Hz, 1H), 6.56 (d, J = 2.0 Hz, 1H), 5.50 (q, J = 7.2 Hz, 1H), 1.72 |
| | (d, J = 7.2 Hz, 3H). NH and OH not observed. |
| 396 | MS m/z 431.0 [M + H]⁺. ¹HNMR (400 MHz, Methanol-d₄) δ 8.25-8.18 (m, |
| | 2H), 8.15 (d, J = 8.0 Hz, 1H), 7.89 (d, J = 8.0 Hz, 1H), 6.59 (d, J = 2.0 Hz, |
| | 1H), 5.13-5.02 (m, 1H), 3.57-3.42 (m, 2H), 2.79-2.67 (m, 1H), 2.51- |
| | 2.34 (m, 1H). NH not observed. |
| 397 | MS m/z 448.0 [M + H]⁺. ¹HNMR (400 MHz, Methanol-d₄) δ 8.22 (d, J = 2.0 |
| | Hz, 1H), 7.97 (d, J = 8.0 Hz, 1H), 7.90 (d, J = 8.4 Hz, 1H), 7.82 (s, 1H), |
| | 6.71 (d, J = 2.0 Hz, 1H), 4.42-4.32 (m, 1H), 2.71-2.64 (m, 2H), 2.37- |
| | 2.29 (m, 2H), 1.44 (s, 3H). NH and OH not observed. |
| 398 | MS m/z 462.1 [M + H]⁺. 1H NMR (400 MHz, Methanol-d₄) δ 8.22 (d, J = 2.0 |
| | Hz, 1H), 7.97 (d, J = 8.0 Hz, 1H), 7.90 (d, J = 8.4 Hz, 1H), 7.82 (s, 1H), |
| | 6.71 (d, J = 2.0 Hz, 1H), 4.16-4.10 (m, 1H), 3.75-3.66 (m, 1H), 2.25- |
| | 2.20 (m, 1H), 2.15-2.08 (m, 1H), 1.84-1.75 (m, 2H), 1.61-1.38 (m, 4H). |
| | NH and OH not observed. |

### Biological Assays

### IL-1β Secretion Assay:

Monocytic THP-1 cells (ATCC: TIB-202) were maintained in growth media consisting of RPMI 1640 medium (ThermoFisher, Cat #11875-085), 10% FBS (ThermoFisher) and 0.05 mM β-mercaptoethanol (ThermoFisher, Cat #21985-023), according to the provider's instructions. The cell concentration was adjusted to 7.5×10⁵ cells/mL, and plated in complete growth media with a final concentration of 100 ng/mL phorbol 12-myristate 13-acetate (PMA, Sigma #P8139). Cells were seeded at 100 L/well into a 96-well cell culture plate (ThermoFisher Cat #165305) and allowed to differentiate for 24 h in a cell culture incubator at 37° C. with 5% CO₂. Cells were washed 1× with 100 ul PBS and replaced with fresh RPMI+5% FBS. Compounds were serial diluted in DMSO with 3 fold dilution for a total of 7 concentrations. Diluted compounds were added to the cells at a ratio of 1:200 and incubated for 20 h. The NLRP3 inflammasome was activated with the addition of 2.5 μM Nigericin (Sigma: Cat #SML1779-1 ml), for 3 h. After incubation, 15 μL of conditioned media was removed and assayed for levels of IL-1β using the HTRF IL-1β assay kit (Cisbio: Cat #62HIL1BPEH) as per the manufacturer's instructions.

Compounds, once produced and prepared according to the present invention, can be assessed in variety of assays to characterize their activities. For example, NLRP3-dependent IL1β secretion was evaluated in THP1 cells. IC50 values of IL1β inhibition were calculated from the plot of percentage of inhibition versus the inhibitor concentration by a logistics fit. TABLE I depict examples of compounds according to generic Formula I. Data which is <1 nM is listed as *****; data 1-10 nM is listed as ****; data 10-100 nM is listed as ***, data 100-300 nM is listed as **, data ≥300 nM is listed as *. The data obtained from the THP1 NLRP3-dependent IL-1β secretion assay demonstrate that the compounds of the present invention could be used to treat diseases mediated through NLRP3 activation.

TABLE I

| Compound | IC50 |
|---|---|
| # | IL-1β Inhibition |
| 1 | **** |
| 2 | ***** |
| 3 | **** |
| 4 | **** |
| 5 | **** |
| 6 | *** |
| 7 | *** |
| 8 | **** |
| 9 | **** |
| 10 | **** |
| 11 | *** |
| 12 | **** |
| 13 | **** |
| 14 | **** |
| 15 | **** |
| 16 | *** |
| 17 | **** |
| 18 | **** |
| 19 | *** |
| 20 | **** |
| 21 | *** |
| 22 | ***** |
| 23 | **** |
| 24 | ***** |
| 25 | **** |
| 26 | *** |
| 27 | **** |
| 28 | **** |
| 29 | **** |
| 30 | **** |
| 31 | **** |
| 32 | **** |
| 33 | **** |
| 34 | **** |
| 35 | *** |
| 36 | **** |
| 37 | ***** |
| 38 | *** |
| 39 | **** |
| 40 | ***** |
| 41 | **** |
| 42 | **** |
| 43 | *** |
| 44 | ***** |
| 45 | *** |
| 46 | *** |
| 47 | *** |
| 48 | ***** |
| 49 | *** |
| 50 | ***** |
| 51 | **** |
| 52 | **** |
| 53 | **** |
| 54 | *** |
| 55 | *** |
| 56 | ***** |
| 57 | *** |
| 58 | *** |
| 59 | ***** |
| 60 | **** |
| 61 | ***** |
| 62 | *** |
| 63 | **** |
| 64 | *** |
| 65 | *** |
| 66 | *** |
| 67 | **** |
| 68 | *** |
| 69 | *** |
| 70 | **** |
| 71 | **** |
| 72 | *** |
| 73 | *** |
| 74 | *** |
| 75 | **** |
| 76 | *** |
| 77 | **** |
| 78 | *** |
| 79 | *** |
| 80 | *** |
| 81 | *** |
| 82 | **** |
| 83 | **** |
| 84 | *** |
| 85 | ***** |
| 86 | ***** |
| 87 | **** |
| 88 | **** |
| 89 | **** |
| 90 | ***** |
| 91 | *** |
| 92 | ** |
| 93 | *** |
| 94 | *** |
| 95 | **** |
| 96 | **** |
| 97 | *** |
| 98 | **** |
| 99 | ** |
| 100 | **** |
| 101 | **** |
| 102 | **** |
| 103 | *** |
| 104 | *** |
| 105 | ** |
| 106 | *** |
| 107 | *** |
| 108 | **** |
| 109 | **** |
| 110 | **** |
| 111 | **** |
| 112 | **** |
| 113 | **** |
| 114 | **** |
| 115 | **** |
| 116 | *** |
| 117 | *** |
| 118 | ***** |
| 119 | ***** |
| 120 | ***** |
| 121 | *** |
| 122 | **** |
| 123 | **** |
| 124 | **** |
| 125 | ** |
| 126 | **** |
| 127 | *** |
| 128 | **** |
| 129 | **** |
| 130 | **** |
| 131 | ***** |
| 132 | *** |
| 133 | *** |
| 134 | **** |
| 135 | ***** |
| 136 | **** |
| 137 | **** |
| 138 | **** |
| 139 | **** |
| 140 | ***** |
| 141 | **** |
| 142 | **** |
| 143 | *** |
| 144 | **** |
| 145 | ***** |
| 146 | **** |
| 147 | **** |
| 148 | ** |
| 149 | **** |
| 150 | ** |
| 151 | **** |
| 152 | **** |
| 153 | **** |
| 154 | **** |
| 155 | *** |
| 156 | **** |
| 157 | **** |
| 158 | **** |
| 159 | ***** |
| 160 | **** |
| 161 | **** |
| 162 | **** |
| 163 | *** |
| 164 | ** |
| 165 | **** |
| 166 | ***** |
| 167 | *** |
| 168 | **** |
| 169 | ***** |
| 170 | ***** |
| 171 | ***** |
| 172 | ***** |
| 173 | ***** |
| 174 | *** |
| 175 | ***** |
| 176 | *** |
| 177 | *** |
| 178 | **** |
| 179 | *** |
| 180 | **** |
| 181 | * |
| 182 | **** |
| 183 | *** |
| 184 | **** |
| 185 | **** |
| 186 | ***** |
| 187 | **** |
| 188 | *** |
| 189 | *** |
| 190 | ***** |
| 191 | ***** |
| 192 | *** |
| 193 | **** |
| 194 | **** |
| 195 | ***** |
| 196 | ***** |
| 197 | ***** |
| 198 | ***** |
| 199 | **** |
| 200 | **** |
| 201 | **** |
| 202 | **** |
| 203 | ***** |
| 204 | **** |
| 205 | *** |
| 206 | ***** |
| 207 | ***** |
| 208 | ***** |
| 209 | ***** |
| 210 | **** |
| 211 | ***** |
| 212 | ***** |
| 213 | **** |
| 214 | **** |
| 215 | *** |
| 216 | **** |
| 217 | ***** |
| 218 | **** |
| 219 | **** |
| 220 | ** |
| 221 | *** |
| 222 | **** |
| 223 | **** |
| 224 | **** |
| 225 | **** |
| 226 | *** |
| 227 | *** |
| 228 | *** |
| 229 | ***** |
| 230 | * |
| 231 | * |
| 232 | ** |
| 233 | **** |
| 234 | *** |
| 235 | **** |
| 236 | **** |
| 237 | **** |
| 238 | **** |
| 239 | ***** |
| 240 | ***** |
| 241 | ***** |
| 242 | ***** |
| 243 | ***** |
| 244 | ***** |
| 245 | **** |
| 246 | ***** |
| 247 | ***** |
| 248 | * |
| 249 | **** |
| 250 | ***** |
| 251 | ***** |
| 252 | **** |
| 253 | **** |
| 254 | *** |
| 255 | **** |
| 256 | ***** |
| 257 | **** |
| 258 | **** |
| 259 | ***** |
| 260 | **** |
| 261 | ***** |
| 262 | ***** |
| 263 | ***** |
| 264 | ***** |
| 265 | ***** |
| 266 | ***** |
| 267 | **** |
| 268 | **** |
| 269 | **** |
| 270 | **** |
| 271 | ***** |
| 272 | **** |
| 273 | **** |
| 274 | *** |
| 275 | *** |
| 276 | *** |
| 277 | **** |
| 278 | ***** |
| 279 | ***** |
| 280 | **** |
| 281 | **** |
| 282 | ***** |
| 283 | **** |
| 284 | *** |
| 285 | *** |
| 286 | **** |
| 287 | **** |
| 288 | **** |
| 289 | **** |
| 290 | *** |
| 291 | **** |
| 292 | *** |
| 293 | *** |
| 294 | **** |
| 295 | ***** |
| 296 | *** |
| 297 | **** |
| 298 | **** |
| 299 | **** |
| 300 | **** |
| 301 | ***** |
| 302 | **** |
| 303 | ***** |
| 304 | **** |
| 305 | *** |
| 306 | **** |
| 307 | **** |
| 308 | ** |
| 309 | *** |
| 310 | * |
| 311 | **** |
| 312 | ***** |
| 313 | **** |
| 314 | **** |
| 315 | ** |
| 316 | *** |
| 317 | **** |
| 318 | **** |
| 319 | *** |
| 320 | *** |
| 321 | *** |
| 322 | *** |
| 323 | **** |
| 324 | *** |
| 325 | *** |
| 326 | *** |
| 327 | *** |
| 328 | *** |
| 329 | **** |
| 330 | *** |
| 331 | *** |
| 332 | *** |
| 333 | **** |
| 334 | *** |
| 335 | *** |
| 336 | *** |
| 337 | *** |
| 338 | ** |
| 339 | *** |
| 340 | **** |
| 341 | *** |
| 342 | *** |
| 343 | ***** |
| 344 | *** |
| 345 | **** |
| 346 | **** |
| 347 | *** |
| 348 | ***** |
| 349 | **** |
| 350 | **** |
| 351 | **** |
| 352 | **** |
| 353 | **** |
| 354 | **** |
| 355 | *** |
| 356 | ***** |
| 357 | ***** |
| 358 | ***** |
| 359 | **** |
| 360 | ***** |
| 361 | **** |
| 362 | *** |
| 363 | **** |
| 364 | *** |
| 365 | **** |
| 366 | **** |
| 367 | **** |
| 368 | *** |
| 369 | *** |
| 370 | *** |
| 371 | **** |
| 372 | *** |
| 373 | ***** |
| 374 | **** |
| 375 | **** |
| 376 | **** |
| 377 | **** |
| 378 | **** |
| 379 | **** |
| 380 | **** |
| 381 | *** |
| 382 | *** |
| 383 | **** |
| 384 | **** |
| 385 | **** |
| 386 | *** |
| 387 | **** |
| 388 | **** |
| 389 | **** |
| 390 | **** |
| 391 | *** |
| 392 | *** |
| 393 | *** |
| 394 | **** |
| 395 | **** |
| 396 | *** |
| 397 | **** |
| 398 | **** |
| 399 | **** |
| 400 | *** |
| 401 | *** |
| 402 | *** |

Without regard to whether a document cited herein was specifically and individually indicated as being incorporated by reference, all documents referred to herein are incorporated by reference into the present application for any and all purposes to the same extent as if each individual reference was fully set forth herein.

Although certain embodiments have been described in detail above, those having ordinary skill in the art will clearly understand that many modifications are possible in the embodiments without departing from the teachings thereof. All such modifications are intended to be encompassed within the scope of the claims presented herein.

1. A compound selected from: 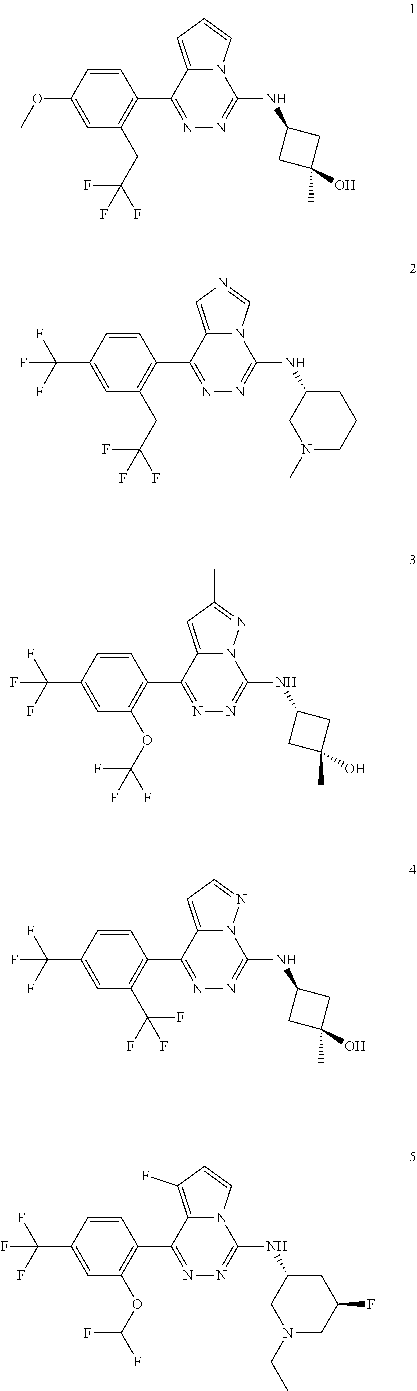 [SMILES: COc1ccc(-c2nnc(N[C@H]3C[C@@](C)(O)C3)n3cccc23)c(CC(F)(F)F)c1 ; CN1CCC[C@@H](Nc2nnc(-c3ccc(C(F)(F)F)cc3CC(F)(F)F)c3cncn23)C1 ; Cc1cc2c(-c3ccc(C(F)(F)F)cc3OC(F)(F)F)nnc(N[C@H]3C[C@@](C)(O)C3)n2n1 ; C[C@]1(O)C[C@@H](Nc2nnc(-c3ccc(C(F)(F)F)cc3C(F)(F)F)c3ccnn23)C1 ; CCN1C[C@H](F)C[C@@H](Nc2nnc(-c3ccc(C(F)(F)F)cc3OC(F)F)c3c(F)ccn23)C1] 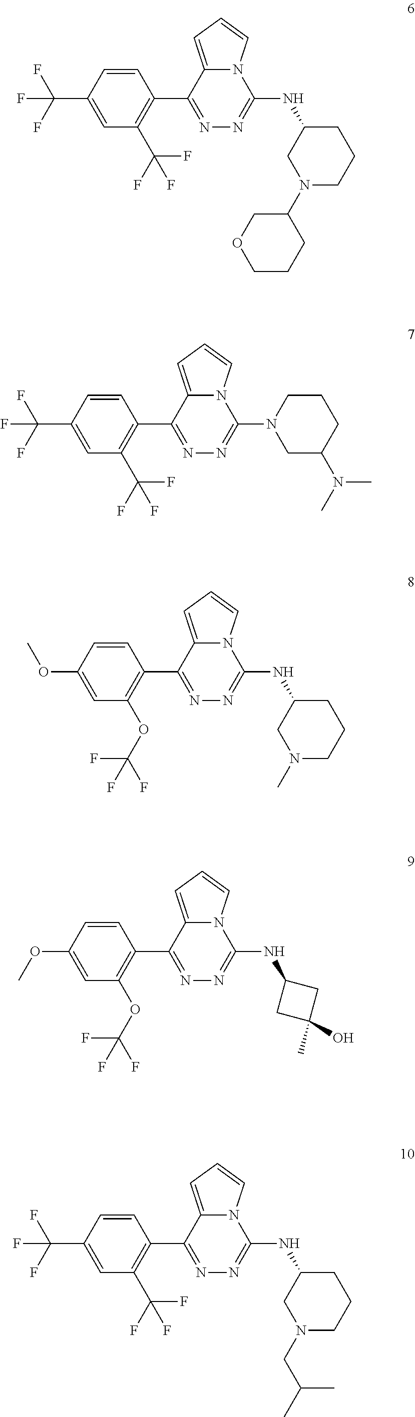 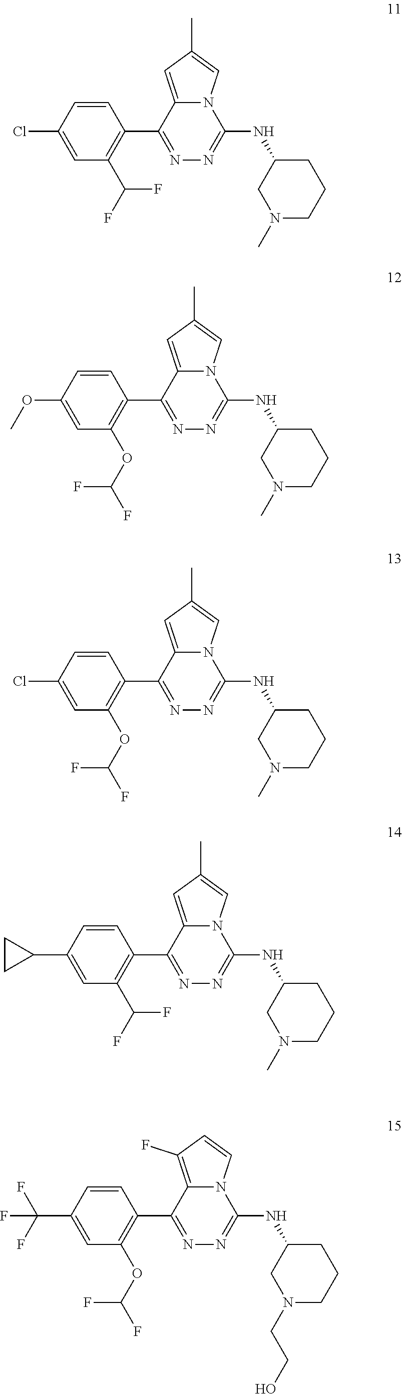 [SMILES: Cc1cc2c(-c3ccc(Cl)cc3C(F)F)nnc(N[C@@H]3CCCN(C)C3)n2c1 ; COc1ccc(-c2nnc(N[C@@H]3CCCN(C)C3)n3cc(C)cc23)c(OC(F)F)c1 ; Cc1cc2c(-c3ccc(Cl)cc3OC(F)F)nnc(N[C@@H]3CCCN(C)C3)n2c1 ; Cc1cc2c(-c3ccc(C4CC4)cc3C(F)F)nnc(N[C@@H]3CCCN(C)C3)n2c1 ; OCCN1CCC[C@@H](Nc2nnc(-c3ccc(C(F)(F)F)cc3OC(F)F)c3c(F)ccn23)C1] 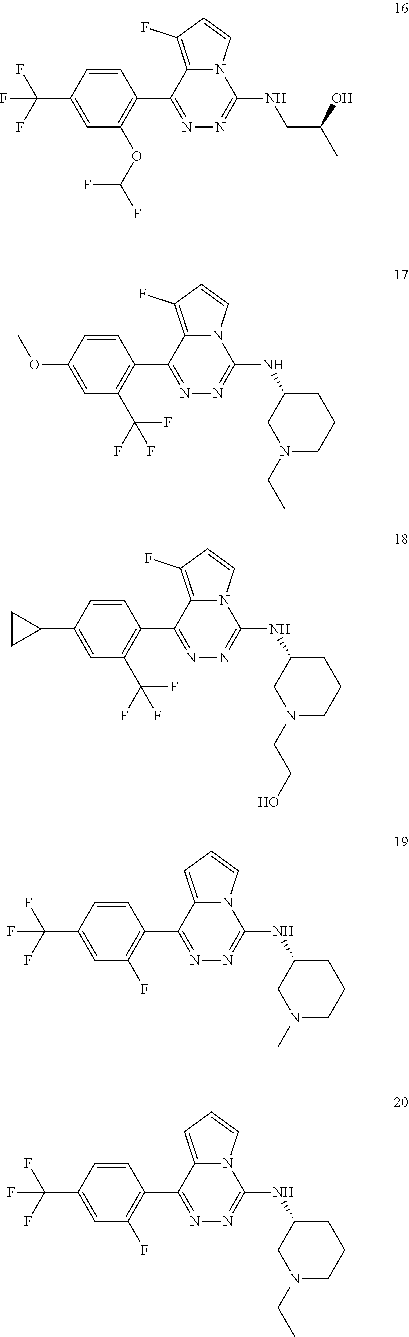 [SMILES: C[C@H](O)CNc1nnc(-c2ccc(C(F)(F)F)cc2OC(F)F)c2c(F)ccn12 ; CCN1CCC[C@@H](Nc2nnc(-c3ccc(OC)cc3C(F)(F)F)c3c(F)ccn23)C1 ; OCCN1CCC[C@@H](Nc2nnc(-c3ccc(C4CC4)cc3C(F)(F)F)c3c(F)ccn23)C1 ; CN1CCC[C@@H](Nc2nnc(-c3ccc(C(F)(F)F)cc3F)c3cccn23)C1 ; CCN1CCC[C@@H](Nc2nnc(-c3ccc(C(F)(F)F)cc3F)c3cccn23)C1] 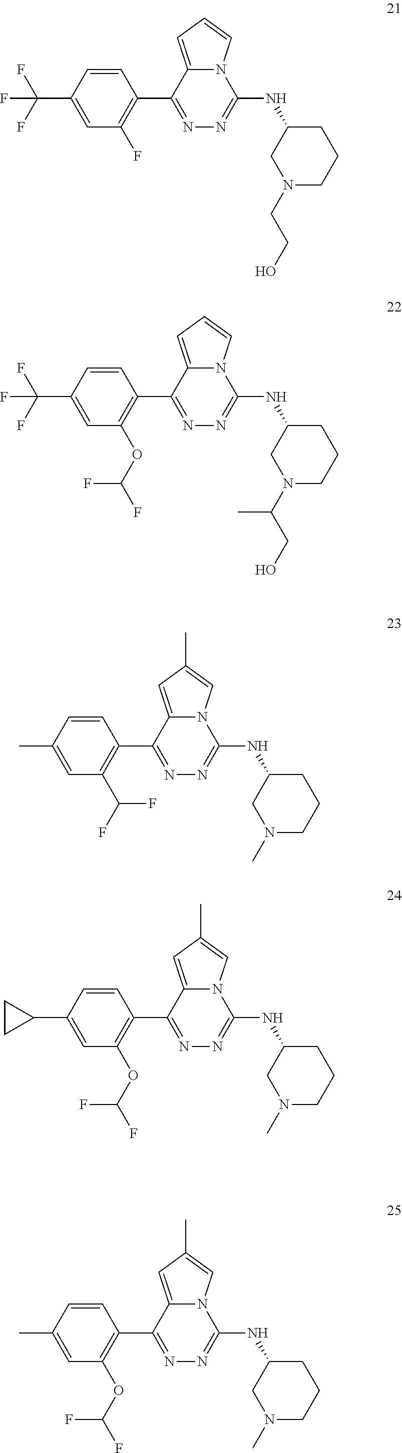 [SMILES: OCCN1CCC[C@@H](Nc2nnc(-c3ccc(C(F)(F)F)cc3F)c3cccn23)C1 ; CC(CO)N1CCC[C@@H](Nc2nnc(-c3ccc(C(F)(F)F)cc3OC(F)F)c3cccn23)C1 ; Cc1ccc(-c2nnc(N[C@@H]3CCCN(C)C3)n3cc(C)cc23)c(C(F)F)c1 ; Cc1cc2c(-c3ccc(C4CC4)cc3OC(F)F)nnc(N[C@@H]3CCCN(C)C3)n2c1 ; Cc1ccc(-c2nnc(N[C@@H]3CCCN(C)C3)n3cc(C)cc23)c(OC(F)F)c1] 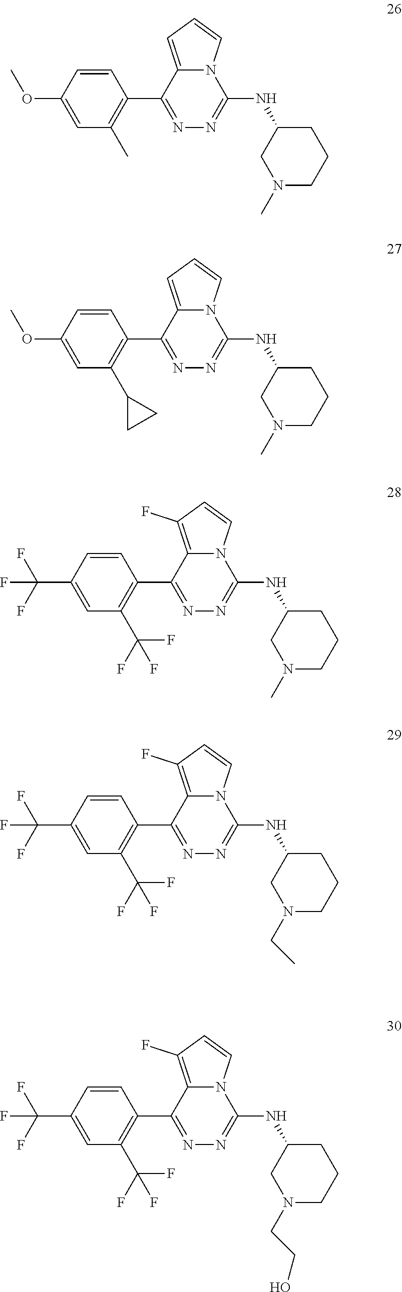 [SMILES: COc1ccc(-c2nnc(N[C@@H]3CCCN(C)C3)n3cccc23)c(C)c1 ; COc1ccc(-c2nnc(N[C@@H]3CCCN(C)C3)n3cccc23)c(C2CC2)c1 ; CN1CCC[C@@H](Nc2nnc(-c3ccc(C(F)(F)F)cc3C(F)(F)F)c3c(F)ccn23)C1 ; CCN1CCC[C@@H](Nc2nnc(-c3ccc(C(F)(F)F)cc3C(F)(F)F)c3c(F)ccn23)C1 ; OCCN1CCC[C@@H](Nc2nnc(-c3ccc(C(F)(F)F)cc3C(F)(F)F)c3c(F)ccn23)C1] 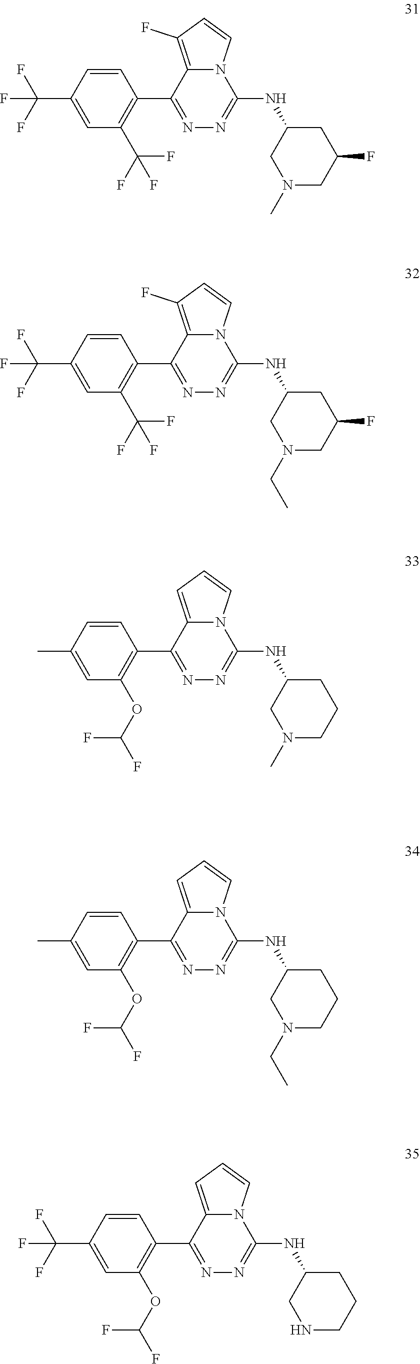 [SMILES: CN1C[C@H](F)C[C@@H](Nc2nnc(-c3ccc(C(F)(F)F)cc3C(F)(F)F)c3c(F)ccn23)C1 ; CCN1C[C@H](F)C[C@@H](Nc2nnc(-c3ccc(C(F)(F)F)cc3C(F)(F)F)c3c(F)ccn23)C1 ; Cc1ccc(-c2nnc(N[C@@H]3CCCN(C)C3)n3cccc23)c(OC(F)F)c1 ; CCN1CCC[C@@H](Nc2nnc(-c3ccc(C)cc3OC(F)F)c3cccn23)C1 ; FC(F)Oc1cc(C(F)(F)F)ccc1-c1nnc(N[C@@H]2CCCNC2)n2cccc12] 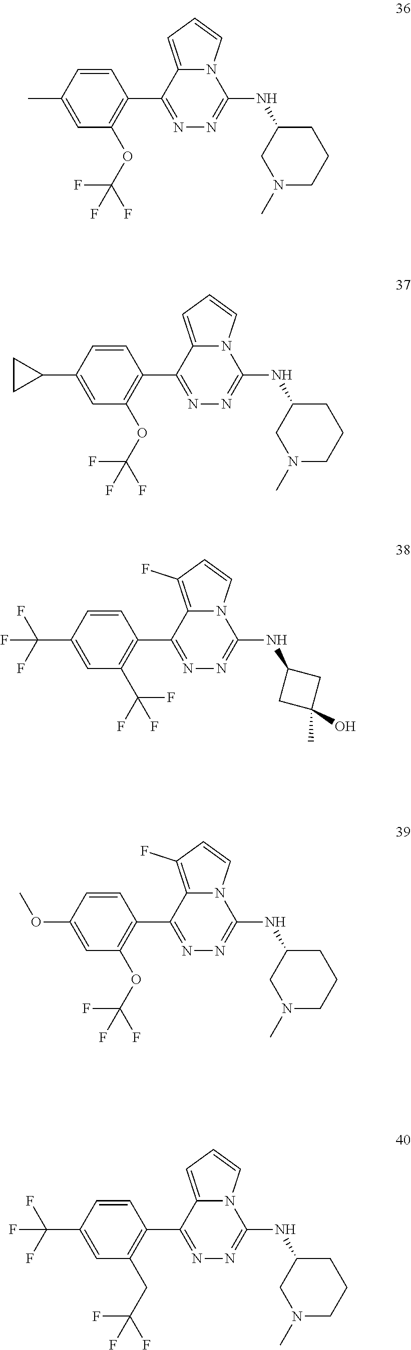 [SMILES: Cc1ccc(-c2nnc(N[C@@H]3CCCN(C)C3)n3cccc23)c(OC(F)(F)F)c1 ; CN1CCC[C@@H](Nc2nnc(-c3ccc(C4CC4)cc3OC(F)(F)F)c3cccn23)C1 ; C[C@]1(O)C[C@@H](Nc2nnc(-c3ccc(C(F)(F)F)cc3C(F)(F)F)c3c(F)ccn23)C1 ; COc1ccc(-c2nnc(N[C@@H]3CCCN(C)C3)n3ccc(F)c23)c(OC(F)(F)F)c1 ; CN1CCC[C@@H](Nc2nnc(-c3ccc(C(F)(F)F)cc3CC(F)(F)F)c3cccn23)C1] 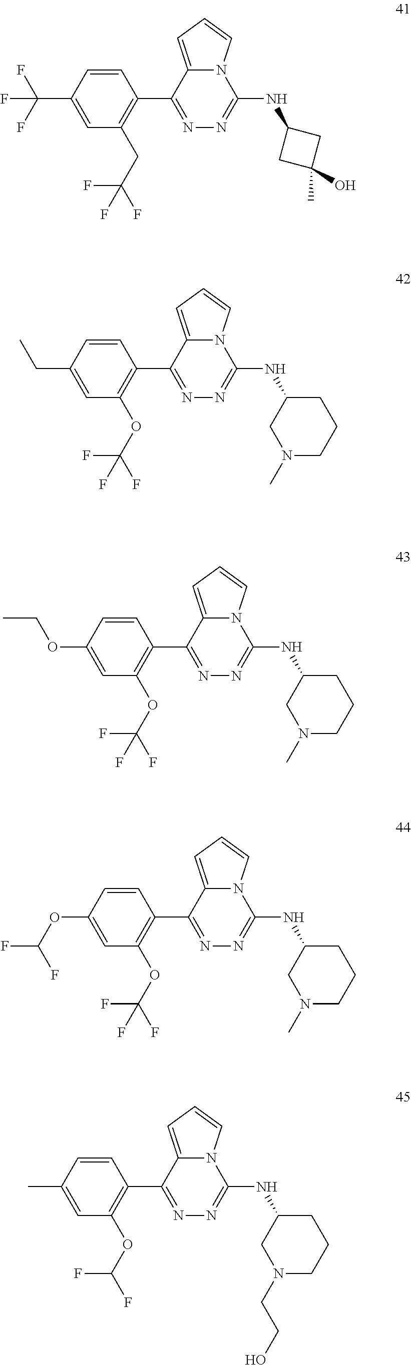 [SMILES: C[C@]1(O)C[C@@H](Nc2nnc(-c3ccc(C(F)(F)F)cc3CC(F)(F)F)c3cccn23)C1 ; CCc1ccc(-c2nnc(N[C@@H]3CCCN(C)C3)n3cccc23)c(OC(F)(F)F)c1 ; CCOc1ccc(-c2nnc(N[C@@H]3CCCN(C)C3)n3cccc23)c(OC(F)(F)F)c1 ; CN1CCC[C@@H](Nc2nnc(-c3ccc(OC(F)F)cc3OC(F)(F)F)c3cccn23)C1 ; Cc1ccc(-c2nnc(N[C@@H]3CCCN(CCO)C3)n3cccc23)c(OC(F)F)c1] 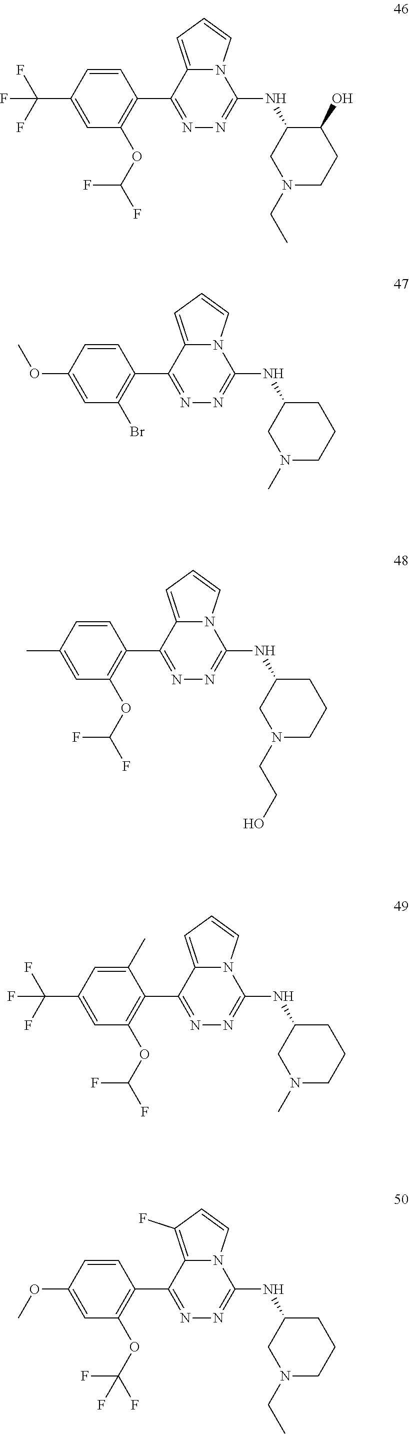 [SMILES: CCN1CC[C@H](O)[C@@H](Nc2nnc(-c3ccc(C(F)(F)F)cc3OC(F)F)c3cccn23)C1 ; COc1ccc(-c2nnc(N[C@@H]3CCCN(C)C3)n3cccc23)c(Br)c1 ; Cc1ccc(-c2nnc(N[C@@H]3CCCN(CCO)C3)n3cccc23)c(OC(F)F)c1 ; Cc1cc(C(F)(F)F)cc(OC(F)F)c1-c1nnc(N[C@@H]2CCCN(C)C2)n2cccc12 ; CCN1CCC[C@@H](Nc2nnc(-c3ccc(OC)cc3OC(F)(F)F)c3c(F)ccn23)C1] 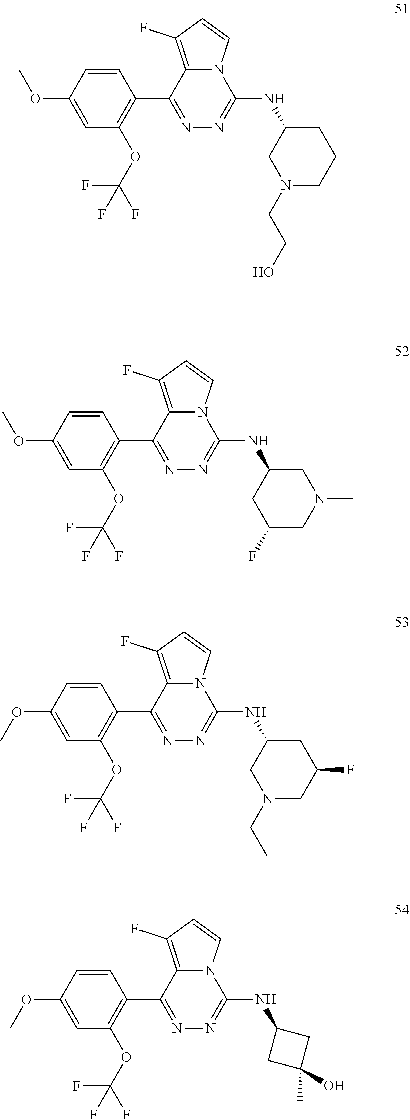 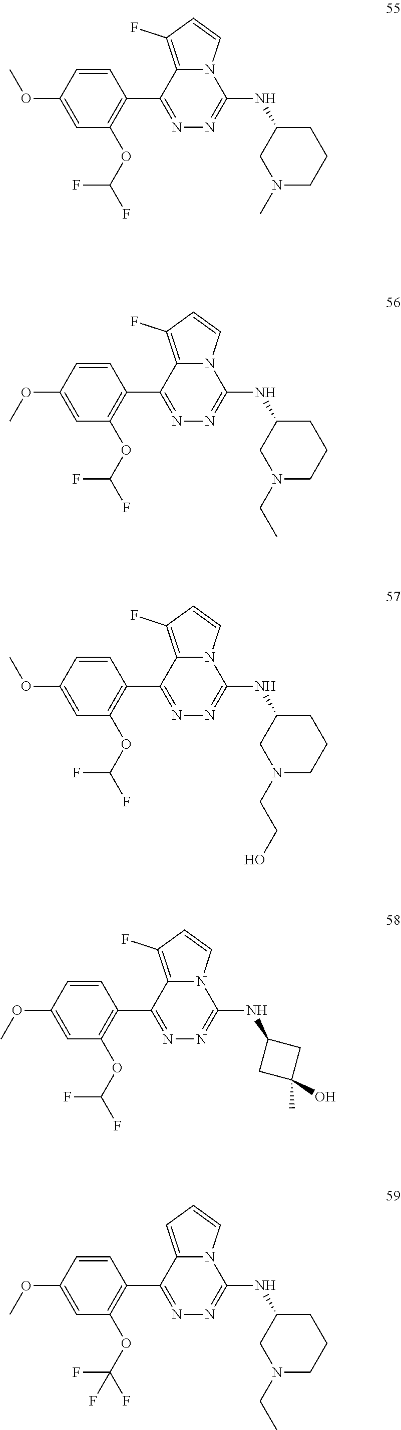 [SMILES: COc1ccc(-c2nnc(N[C@@H]3CCCN(C)C3)n3ccc(F)c23)c(OC(F)F)c1 ; CCN1CCC[C@@H](Nc2nnc(-c3ccc(OC)cc3OC(F)F)c3c(F)ccn23)C1 ; COc1ccc(-c2nnc(N[C@@H]3CCCN(CCO)C3)n3ccc(F)c23)c(OC(F)F)c1 ; COc1ccc(-c2nnc(N[C@H]3C[C@@](C)(O)C3)n3ccc(F)c23)c(OC(F)F)c1 ; CCN1CCC[C@@H](Nc2nnc(-c3ccc(OC)cc3OC(F)(F)F)c3cccn23)C1] 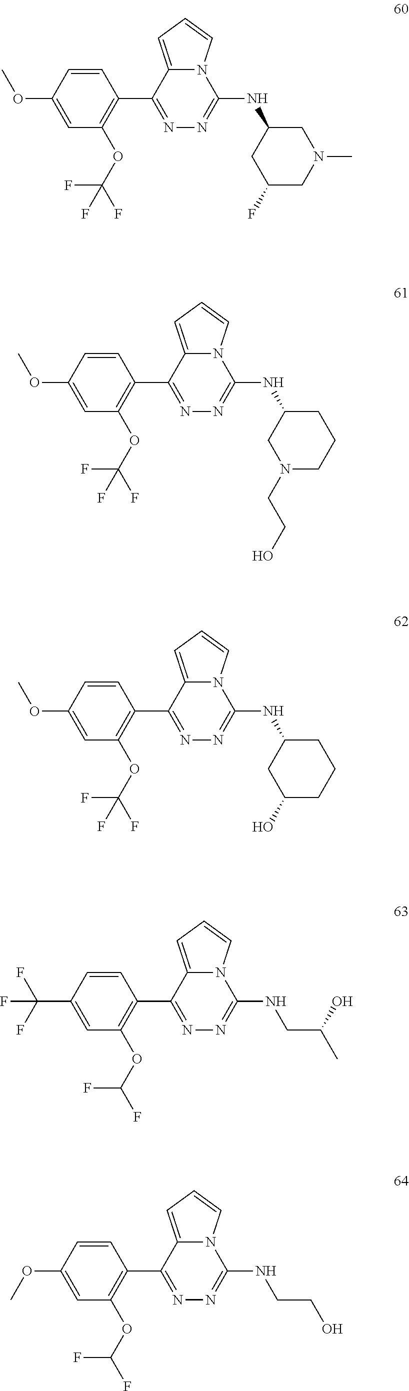 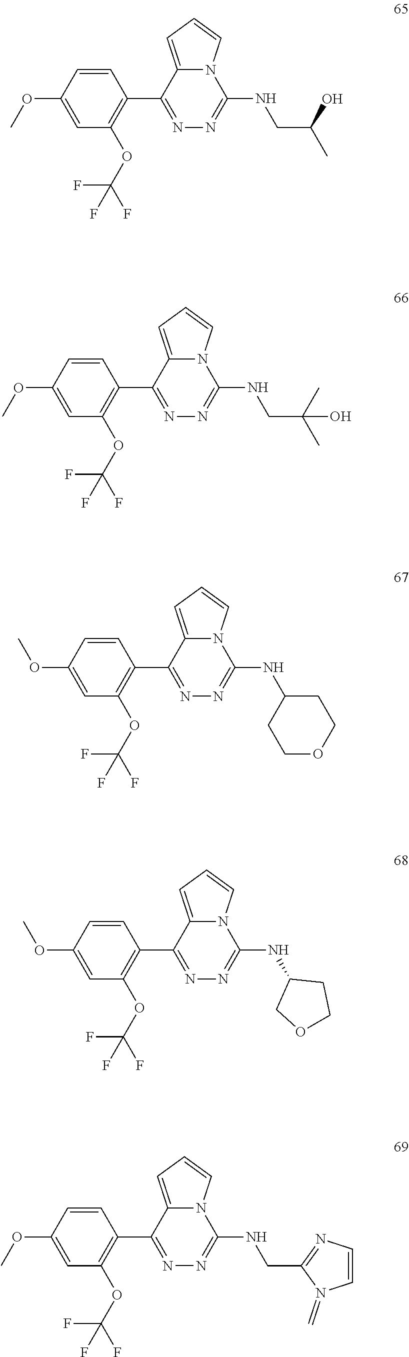 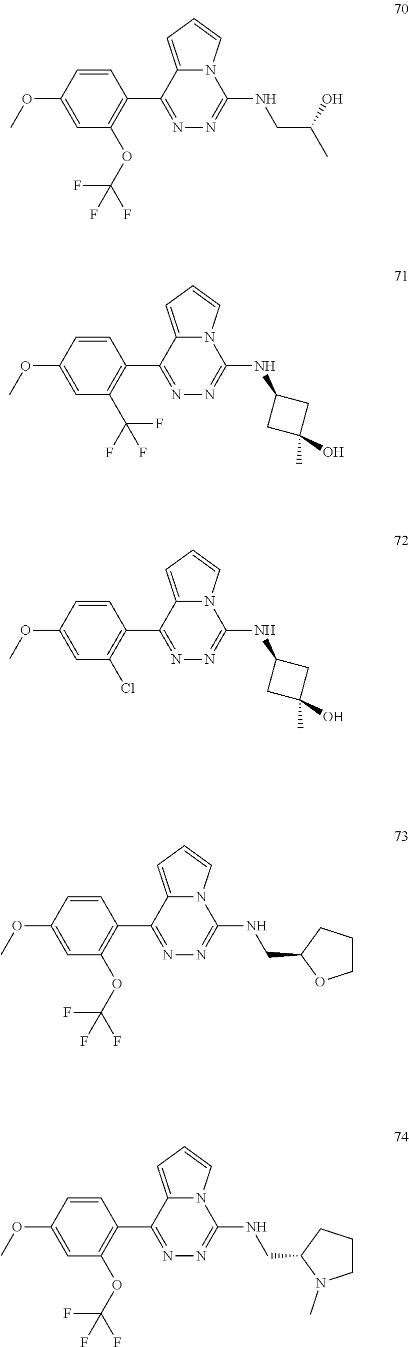 [SMILES: COc1ccc(-c2nnc(NC[C@@H](C)O)n3cccc23)c(OC(F)(F)F)c1 ; COc1ccc(-c2nnc(N[C@H]3C[C@@](C)(O)C3)n3cccc23)c(C(F)(F)F)c1 ; COc1ccc(-c2nnc(N[C@H]3C[C@@](C)(O)C3)n3cccc23)c(Cl)c1 ; COc1ccc(-c2nnc(NC[C@H]3CCCO3)n3cccc23)c(OC(F)(F)F)c1 ; COc1ccc(-c2nnc(NC[C@@H]3CCCN3C)n3cccc23)c(OC(F)(F)F)c1] 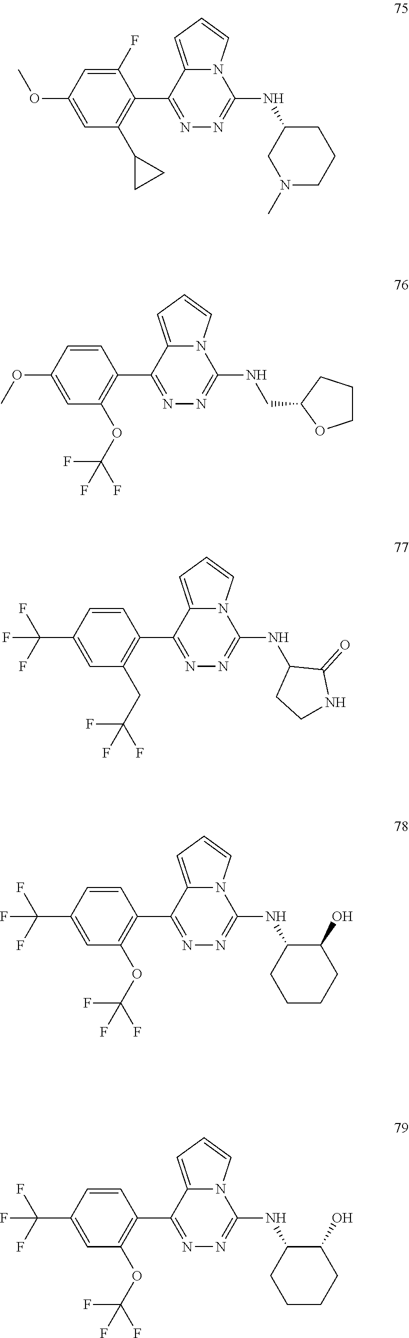 [SMILES: COc1cc(F)c(-c2nnc(N[C@@H]3CCCN(C)C3)n3cccc23)c(C2CC2)c1 ; COc1ccc(-c2nnc(NC[C@@H]3CCCO3)n3cccc23)c(OC(F)(F)F)c1 ; O=C1NCCC1Nc1nnc(-c2ccc(C(F)(F)F)cc2CC(F)(F)F)c2cccn12 ; O[C@H]1CCCC[C@@H]1Nc1nnc(-c2ccc(C(F)(F)F)cc2OC(F)(F)F)c2cccn12 ; O[C@@H]1CCCC[C@@H]1Nc1nnc(-c2ccc(C(F)(F)F)cc2OC(F)(F)F)c2cccn12] 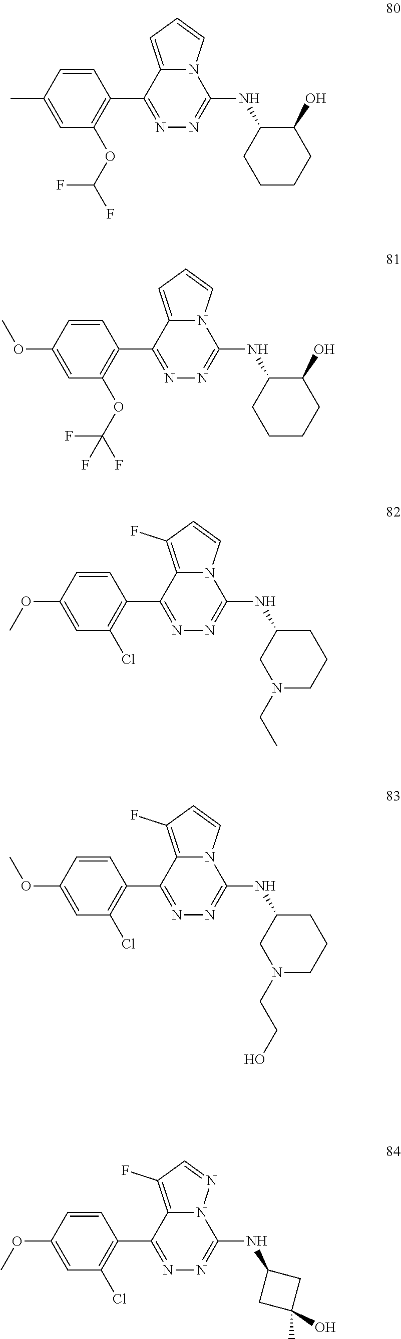 [SMILES: Cc1ccc(-c2nnc(N[C@H]3CCCC[C@@H]3O)n3cccc23)c(OC(F)F)c1 ; COc1ccc(-c2nnc(N[C@H]3CCCC[C@@H]3O)n3cccc23)c(OC(F)(F)F)c1 ; CCN1CCC[C@@H](Nc2nnc(-c3ccc(OC)cc3Cl)c3c(F)ccn23)C1 ; COc1ccc(-c2nnc(N[C@@H]3CCCN(CCO)C3)n3ccc(F)c23)c(Cl)c1 ; COc1ccc(-c2nnc(N[C@H]3C[C@@](C)(O)C3)n3ncc(F)c23)c(Cl)c1] 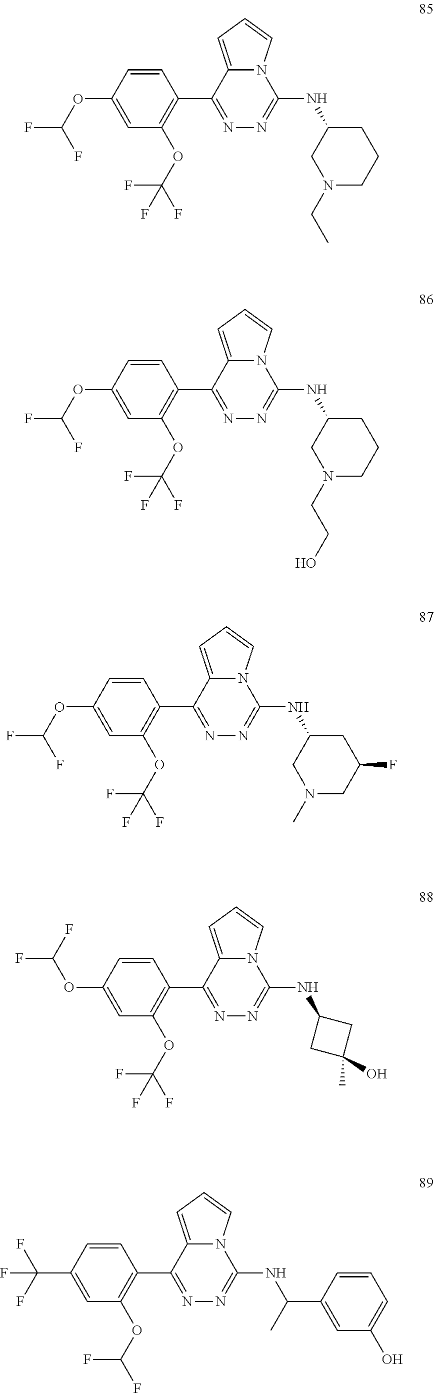 [SMILES: CCN1CCC[C@@H](Nc2nnc(-c3ccc(OC(F)F)cc3OC(F)(F)F)c3cccn23)C1 ; OCCN1CCC[C@@H](Nc2nnc(-c3ccc(OC(F)F)cc3OC(F)(F)F)c3cccn23)C1 ; CN1C[C@H](F)C[C@@H](Nc2nnc(-c3ccc(OC(F)F)cc3OC(F)(F)F)c3cccn23)C1 ; C[C@]1(O)C[C@@H](Nc2nnc(-c3ccc(OC(F)F)cc3OC(F)(F)F)c3cccn23)C1 ; CC(Nc1nnc(-c2ccc(C(F)(F)F)cc2OC(F)F)c2cccn12)c1cccc(O)c1] 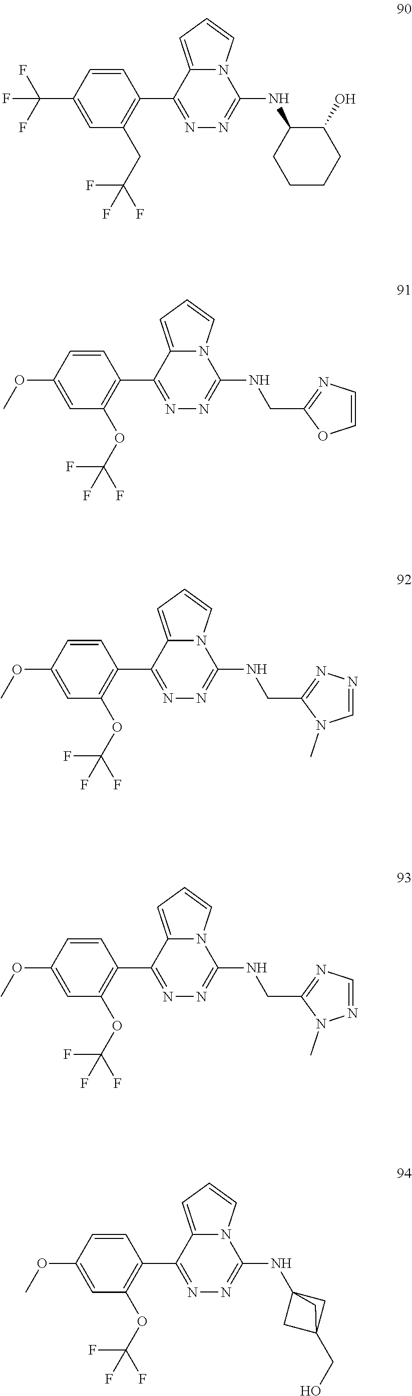 [SMILES: O[C@@H]1CCCC[C@H]1Nc1nnc(-c2ccc(C(F)(F)F)cc2CC(F)(F)F)c2cccn12 ; COc1ccc(-c2nnc(NCc3ncco3)n3cccc23)c(OC(F)(F)F)c1 ; COc1ccc(-c2nnc(NCc3nncn3C)n3cccc23)c(OC(F)(F)F)c1 ; COc1ccc(-c2nnc(NCc3ncnn3C)n3cccc23)c(OC(F)(F)F)c1 ; COc1ccc(-c2nnc(NC34CC(CO)(C3)C4)n3cccc23)c(OC(F)(F)F)c1] 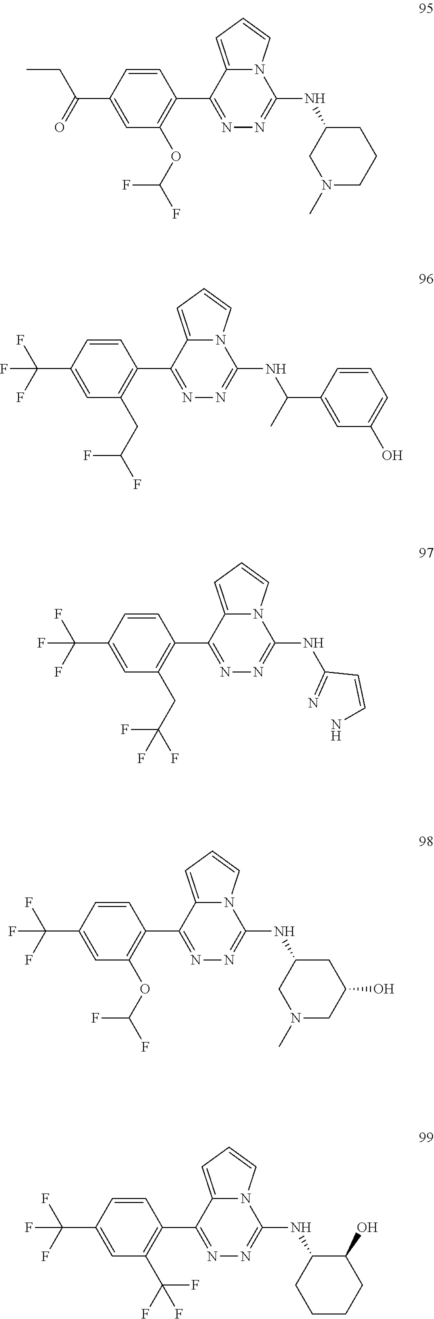 [SMILES: CCC(=O)c1ccc(-c2nnc(N[C@@H]3CCCN(C)C3)n3cccc23)c(OC(F)F)c1 ; CC(Nc1nnc(-c2ccc(C(F)(F)F)cc2CC(F)F)c2cccn12)c1cccc(O)c1 ; N=C(/C=C\N)Nc1nnc(-c2ccc(C(F)(F)F)cc2CC(F)(F)F)c2cccn12 ; CN1C[C@@H](O)C[C@@H](Nc2nnc(-c3ccc(C(F)(F)F)cc3OC(F)F)c3cccn23)C1 ; O[C@H]1CCCC[C@@H]1Nc1nnc(-c2ccc(C(F)(F)F)cc2C(F)(F)F)c2cccn12] 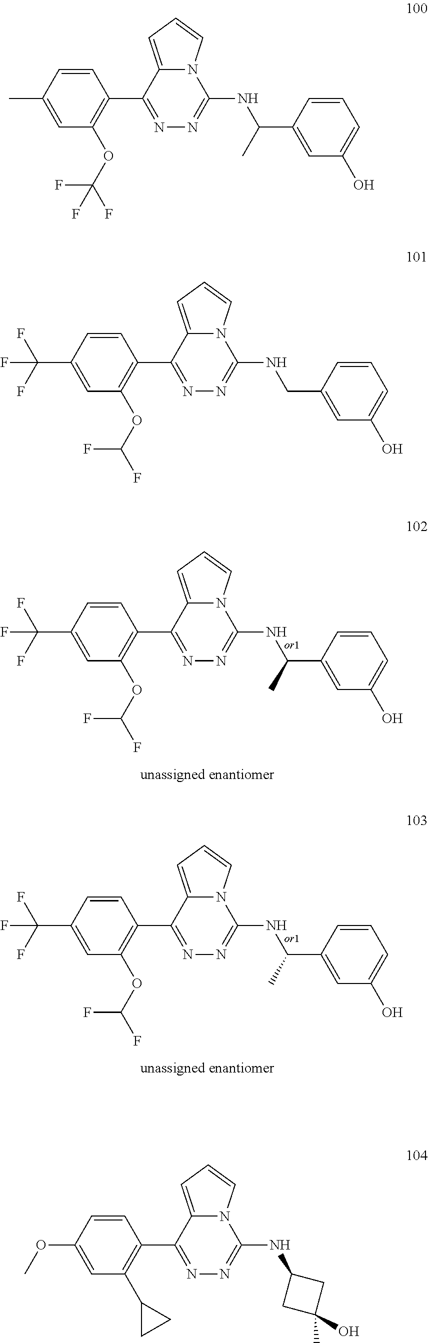 [SMILES: Cc1ccc(-c2nnc(NC(C)c3cccc(O)c3)n3cccc23)c(OC(F)(F)F)c1 ; Oc1cccc(CNc2nnc(-c3ccc(C(F)(F)F)cc3OC(F)F)c3cccn23)c1 ; C[C@@H](Nc1nnc(-c2ccc(C(F)(F)F)cc2OC(F)F)c2cccn12)c1cccc(O)c1 ; C[C@H](Nc1nnc(-c2ccc(C(F)(F)F)cc2OC(F)F)c2cccn12)c1cccc(O)c1 ; COc1ccc(-c2nnc(N[C@H]3C[C@@](C)(O)C3)n3cccc23)c(C2CC2)c1] 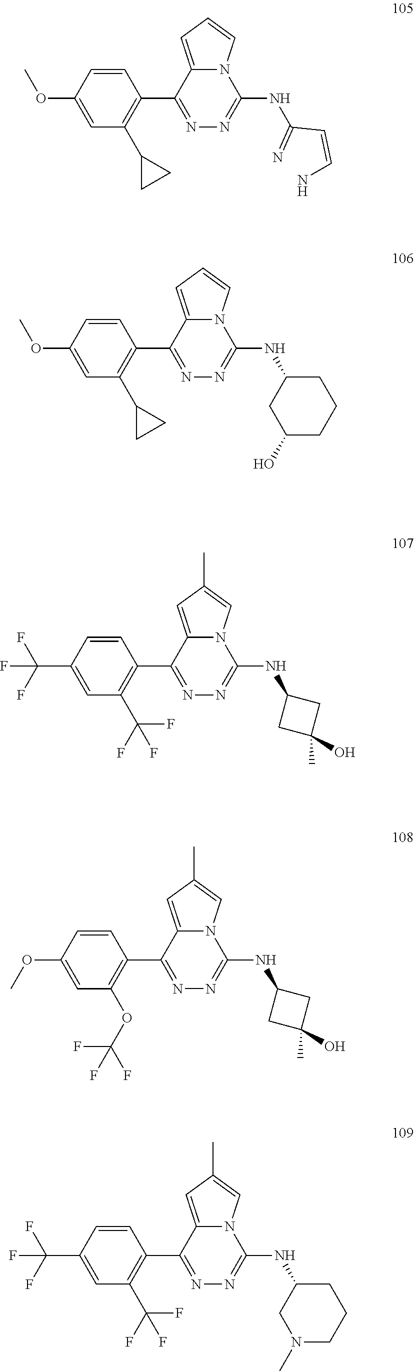 [SMILES: COc1ccc(-c2nnc(NC(=N)/C=C\N)n3cccc23)c(C2CC2)c1 ; COc1ccc(-c2nnc(N[C@@H]3CCC[C@H](O)C3)n3cccc23)c(C2CC2)c1 ; Cc1cc2c(-c3ccc(C(F)(F)F)cc3C(F)(F)F)nnc(N[C@H]3C[C@@](C)(O)C3)n2c1 ; COc1ccc(-c2nnc(N[C@H]3C[C@@](C)(O)C3)n3cc(C)cc23)c(OC(F)(F)F)c1 ; Cc1cc2c(-c3ccc(C(F)(F)F)cc3C(F)(F)F)nnc(N[C@@H]3CCCN(C)C3)n2c1] 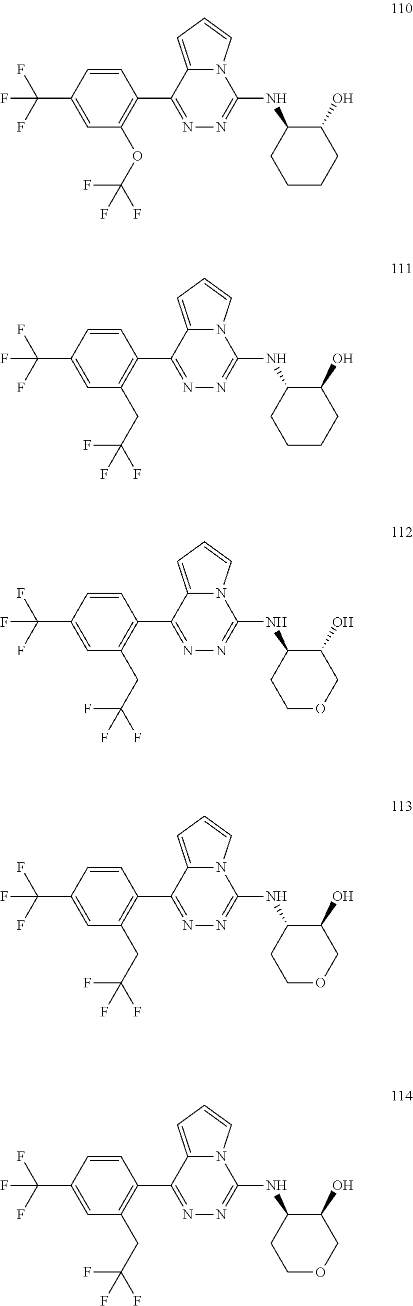 [SMILES: O[C@@H]1CCCC[C@H]1Nc1nnc(-c2ccc(C(F)(F)F)cc2OC(F)(F)F)c2cccn12 ; O[C@H]1CCCC[C@@H]1Nc1nnc(-c2ccc(C(F)(F)F)cc2CC(F)(F)F)c2cccn12 ; O[C@@H]1COCC[C@H]1Nc1nnc(-c2ccc(C(F)(F)F)cc2CC(F)(F)F)c2cccn12 ; O[C@H]1COCC[C@@H]1Nc1nnc(-c2ccc(C(F)(F)F)cc2CC(F)(F)F)c2cccn12 ; O[C@H]1COCC[C@H]1Nc1nnc(-c2ccc(C(F)(F)F)cc2CC(F)(F)F)c2cccn12] 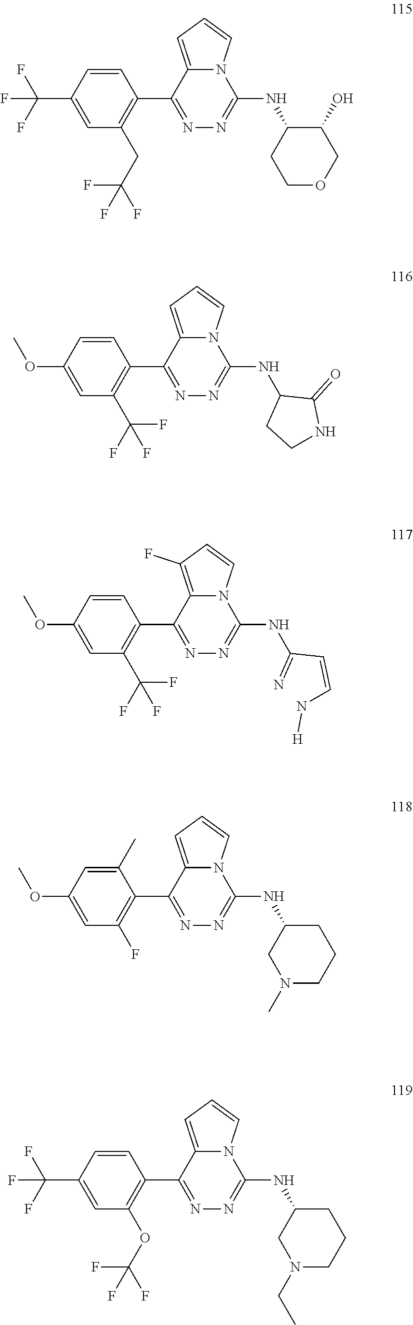 [SMILES: O[C@@H]1COCC[C@@H]1Nc1nnc(-c2ccc(C(F)(F)F)cc2CC(F)(F)F)c2cccn12 ; COc1ccc(-c2nnc(NC3CCNC3=O)n3cccc23)c(C(F)(F)F)c1 ; COc1ccc(-c2nnc(NC(=N)/C=C\N)n3ccc(F)c23)c(C(F)(F)F)c1 ; COc1cc(C)c(-c2nnc(N[C@@H]3CCCN(C)C3)n3cccc23)c(F)c1 ; CCN1CCC[C@@H](Nc2nnc(-c3ccc(C(F)(F)F)cc3OC(F)(F)F)c3cccn23)C1] 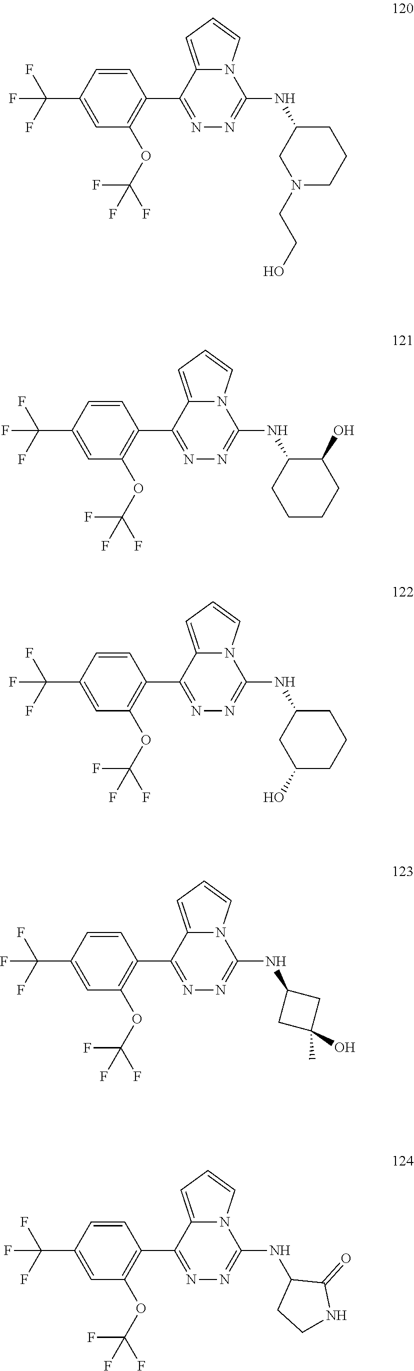 [SMILES: OCCN1CCC[C@@H](Nc2nnc(-c3ccc(C(F)(F)F)cc3OC(F)(F)F)c3cccn23)C1 ; O[C@H]1CCCC[C@@H]1Nc1nnc(-c2ccc(C(F)(F)F)cc2OC(F)(F)F)c2cccn12 ; O[C@H]1CCC[C@@H](Nc2nnc(-c3ccc(C(F)(F)F)cc3OC(F)(F)F)c3cccn23)C1 ; C[C@]1(O)C[C@@H](Nc2nnc(-c3ccc(C(F)(F)F)cc3OC(F)(F)F)c3cccn23)C1 ; O=C1NCCC1Nc1nnc(-c2ccc(C(F)(F)F)cc2OC(F)(F)F)c2cccn12] 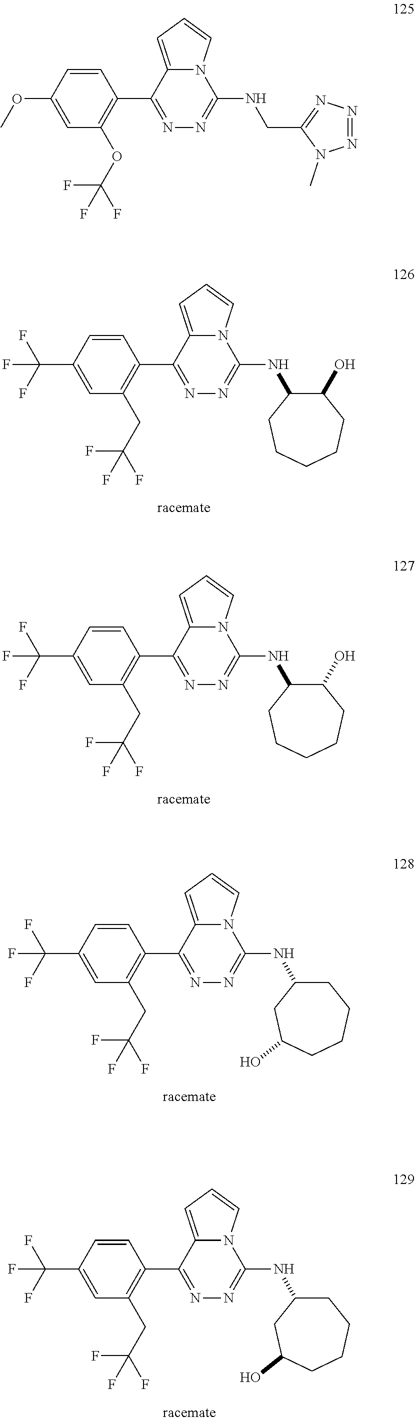 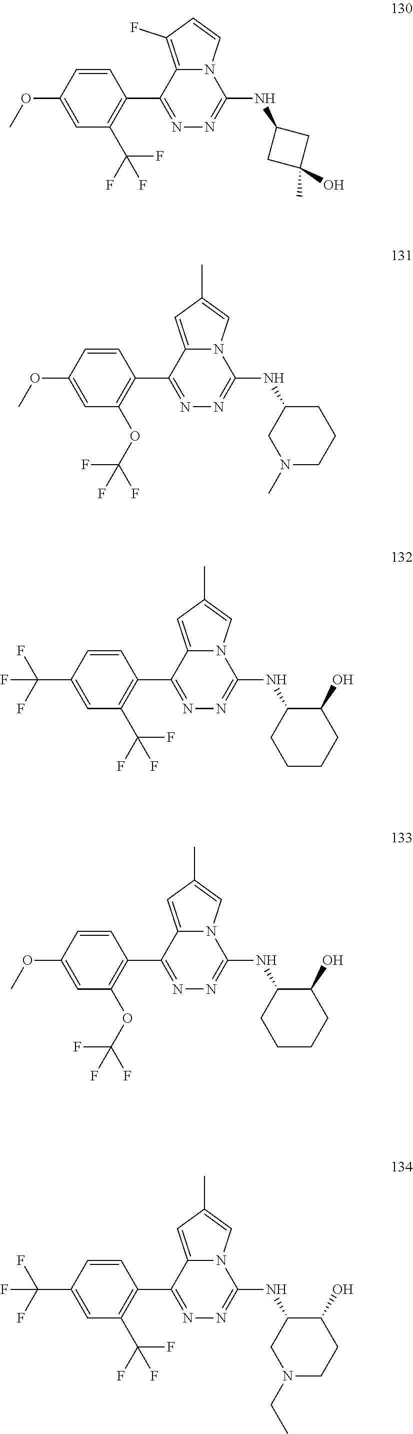 [SMILES: COc1ccc(-c2nnc(N[C@H]3C[C@@](C)(O)C3)n3ccc(F)c23)c(C(F)(F)F)c1 ; COc1ccc(-c2nnc(N[C@@H]3CCCN(C)C3)n3cc(C)cc23)c(OC(F)(F)F)c1 ; Cc1cc2c(-c3ccc(C(F)(F)F)cc3C(F)(F)F)nnc(N[C@H]3CCCC[C@@H]3O)n2c1 ; COc1ccc(-c2nnc(N[C@H]3CCCC[C@@H]3O)n3cc(C)cc23)c(OC(F)(F)F)c1 ; CCN1CC[C@@H](O)[C@@H](Nc2nnc(-c3ccc(C(F)(F)F)cc3C(F)(F)F)c3cc(C)cn23)C1] 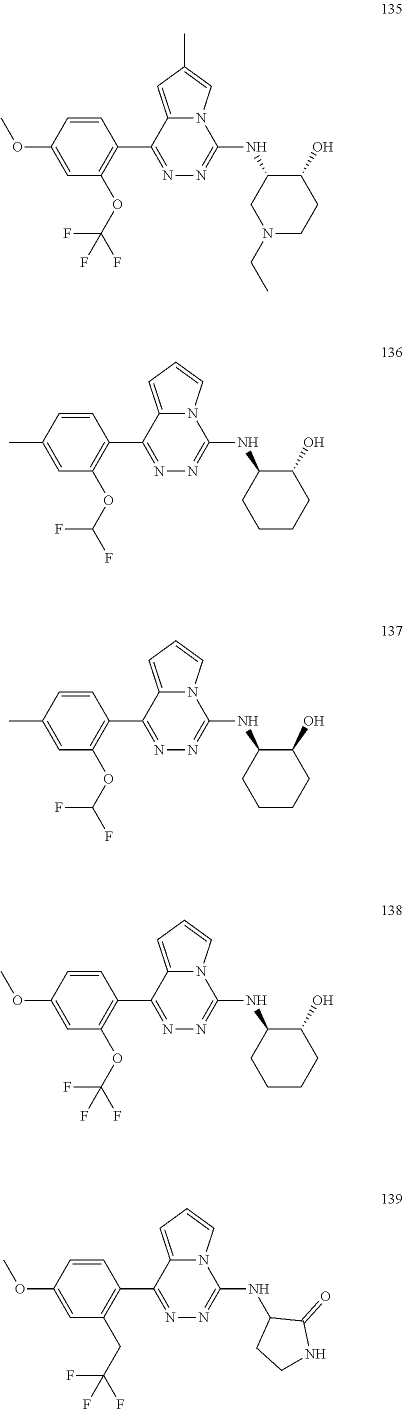 [SMILES: CCN1CC[C@@H](O)[C@@H](Nc2nnc(-c3ccc(OC)cc3OC(F)(F)F)c3cc(C)cn23)C1 ; Cc1ccc(-c2nnc(N[C@@H]3CCCC[C@H]3O)n3cccc23)c(OC(F)F)c1 ; Cc1ccc(-c2nnc(N[C@@H]3CCCC[C@@H]3O)n3cccc23)c(OC(F)F)c1 ; COc1ccc(-c2nnc(N[C@@H]3CCCC[C@H]3O)n3cccc23)c(OC(F)(F)F)c1 ; COc1ccc(-c2nnc(NC3CCNC3=O)n3cccc23)c(CC(F)(F)F)c1] 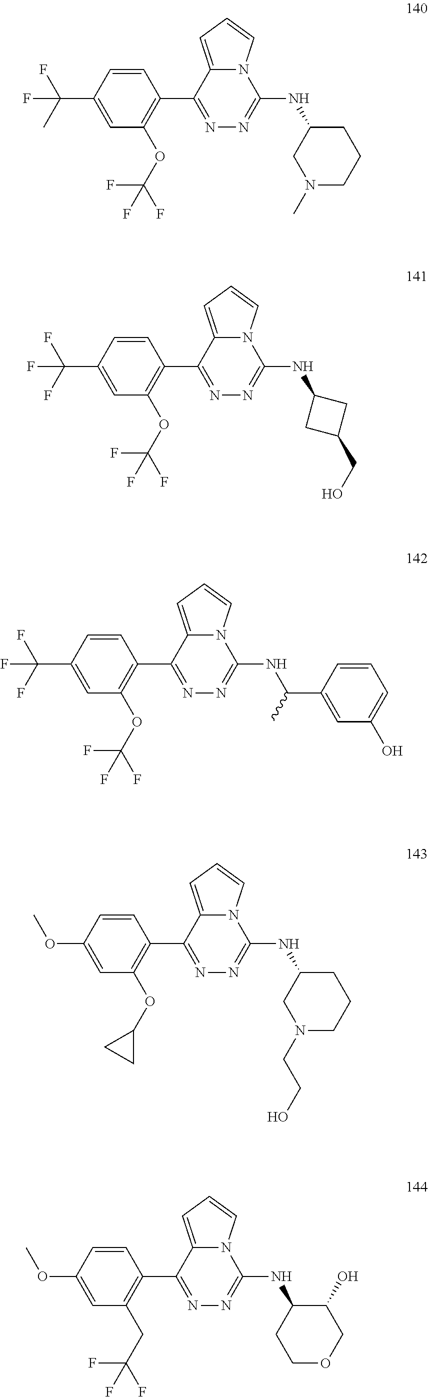 [SMILES: CN1CCC[C@@H](Nc2nnc(-c3ccc(C(C)(F)F)cc3OC(F)(F)F)c3cccn23)C1 ; OC[C@H]1C[C@@H](Nc2nnc(-c3ccc(C(F)(F)F)cc3OC(F)(F)F)c3cccn23)C1 ; CC(Nc1nnc(-c2ccc(C(F)(F)F)cc2OC(F)(F)F)c2cccn12)c1cccc(O)c1 ; COc1ccc(-c2nnc(N[C@@H]3CCCN(CCO)C3)n3cccc23)c(OC2CC2)c1 ; COc1ccc(-c2nnc(N[C@@H]3CCOC[C@H]3O)n3cccc23)c(CC(F)(F)F)c1] 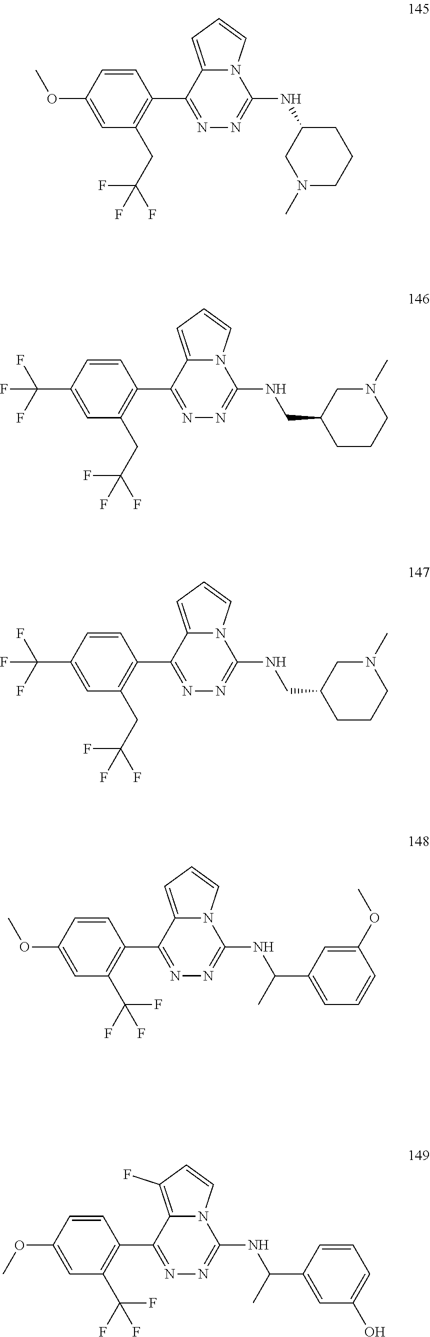 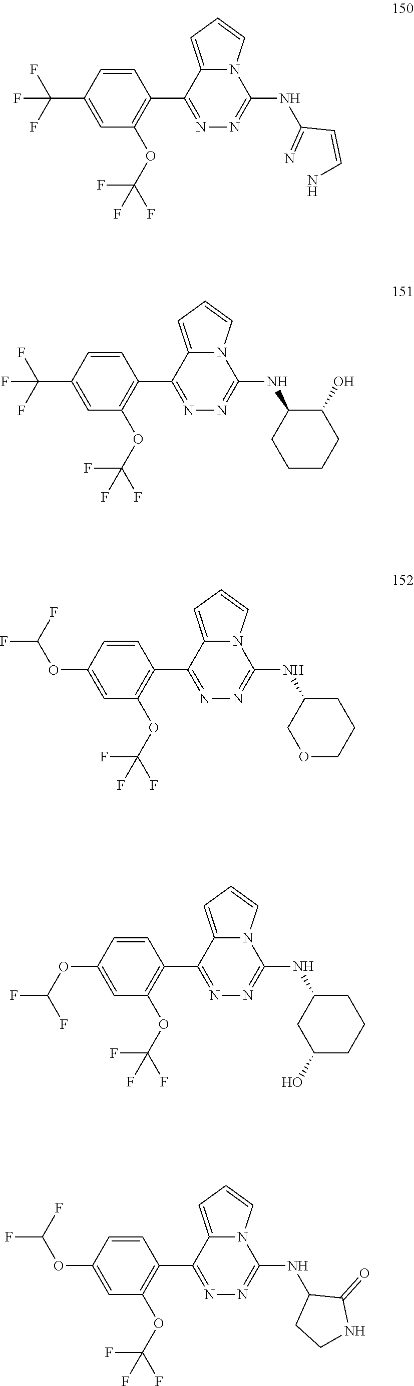 [SMILES: N=C(/C=C\N)Nc1nnc(-c2ccc(C(F)(F)F)cc2OC(F)(F)F)c2cccn12 ; O[C@@H]1CCCC[C@H]1Nc1nnc(-c2ccc(C(F)(F)F)cc2OC(F)(F)F)c2cccn12 ; FC(F)Oc1ccc(-c2nnc(N[C@@H]3CCCOC3)n3cccc23)c(OC(F)(F)F)c1 ; O[C@H]1CCC[C@@H](Nc2nnc(-c3ccc(OC(F)F)cc3OC(F)(F)F)c3cccn23)C1 ; O=C1NCCC1Nc1nnc(-c2ccc(OC(F)F)cc2OC(F)(F)F)c2cccn12] 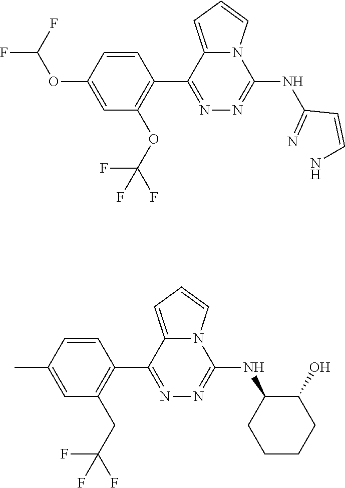 [SMILES: N=C(/C=C\N)Nc1nnc(-c2ccc(OC(F)F)cc2OC(F)(F)F)c2cccn12 ; Cc1ccc(-c2nnc(N[C@@H]3CCCC[C@H]3O)n3cccc23)c(CC(F)(F)F)c1] 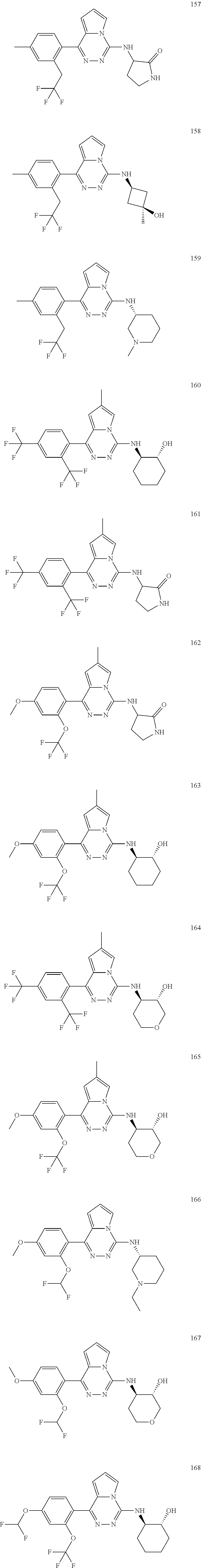 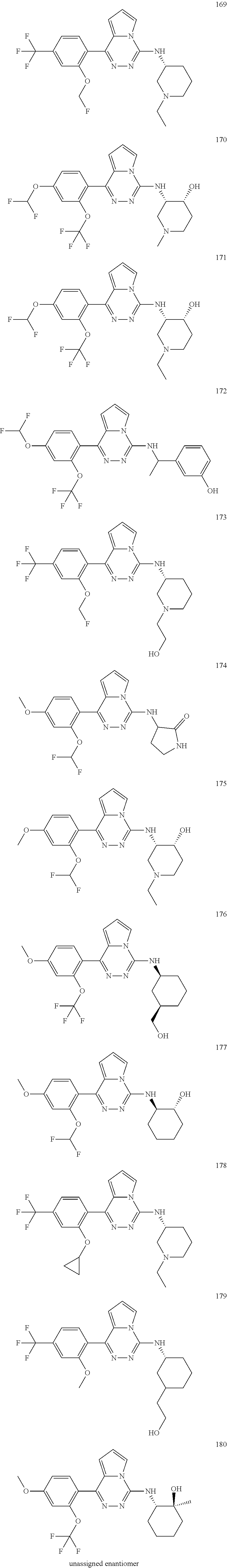 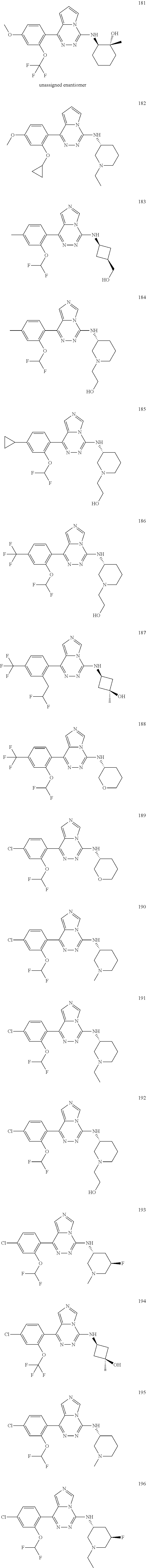 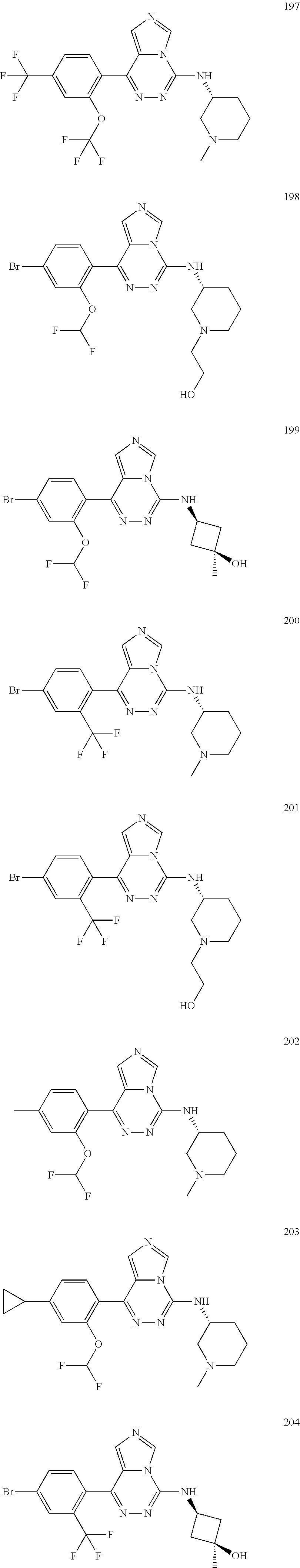 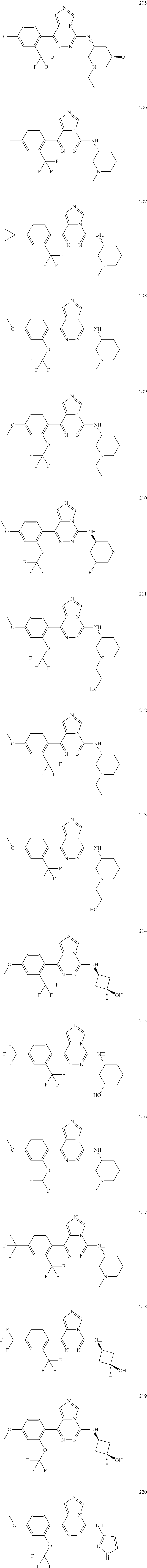 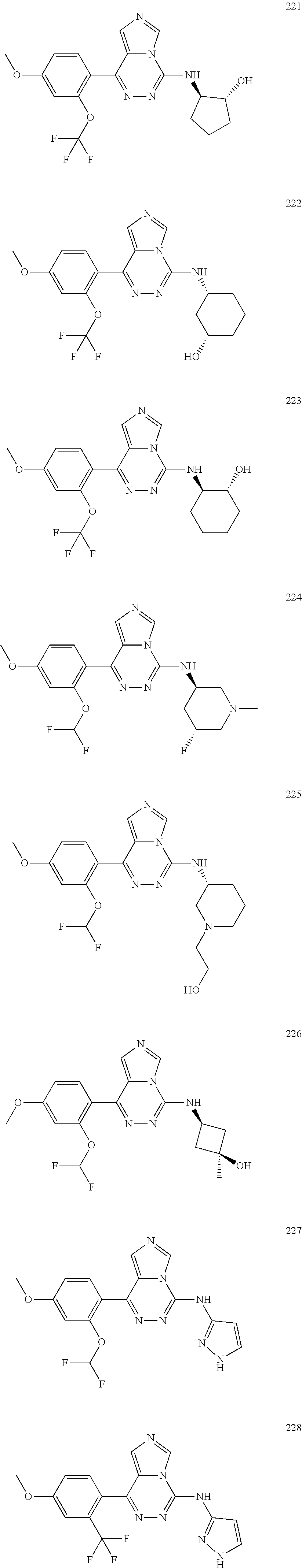 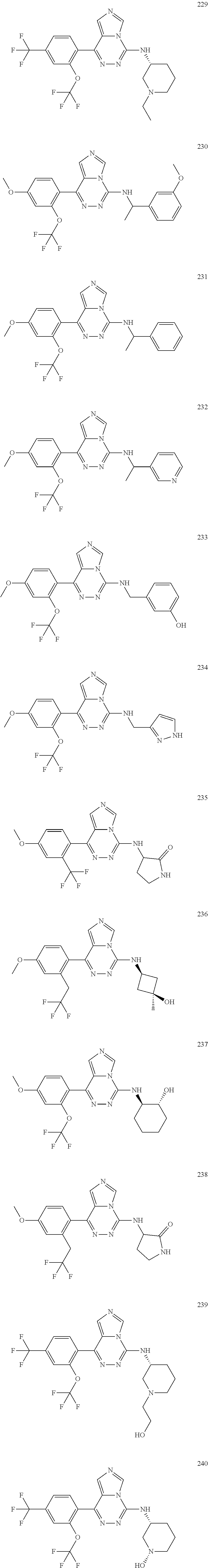 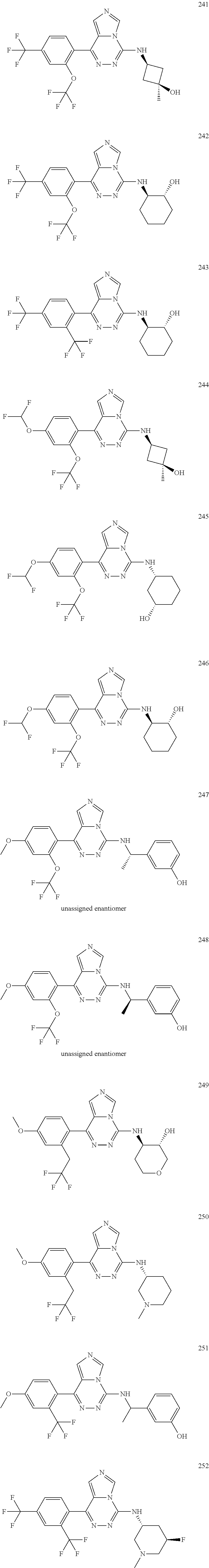 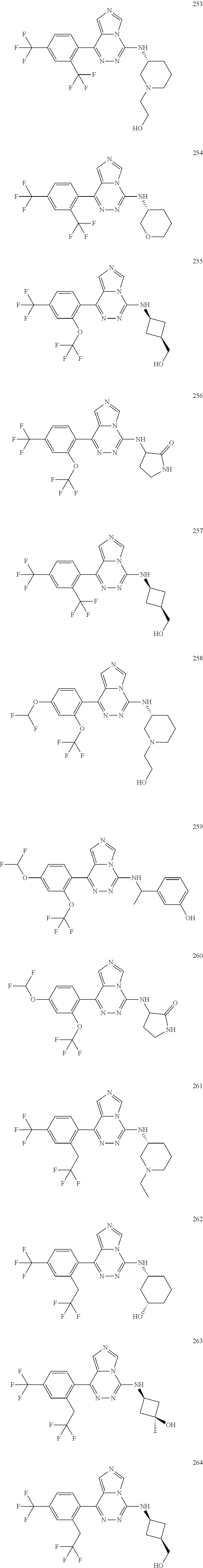 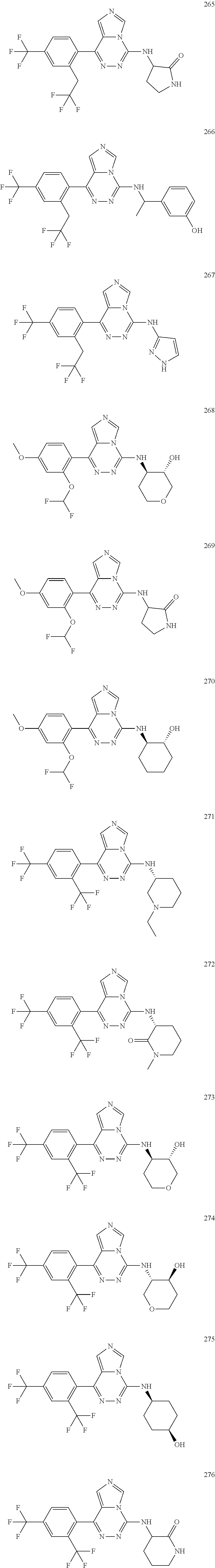 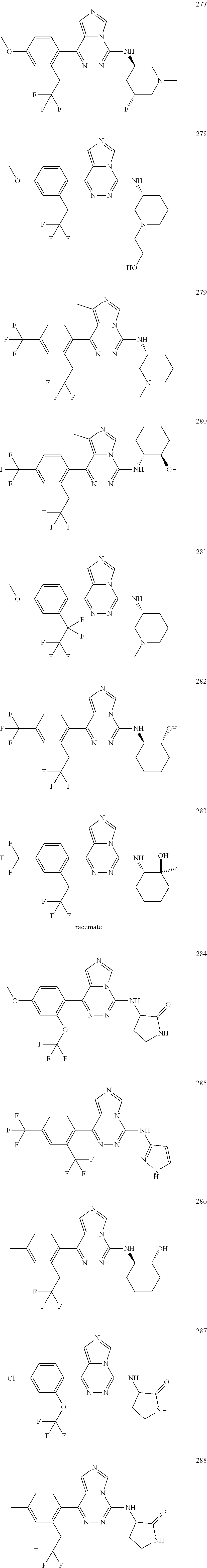 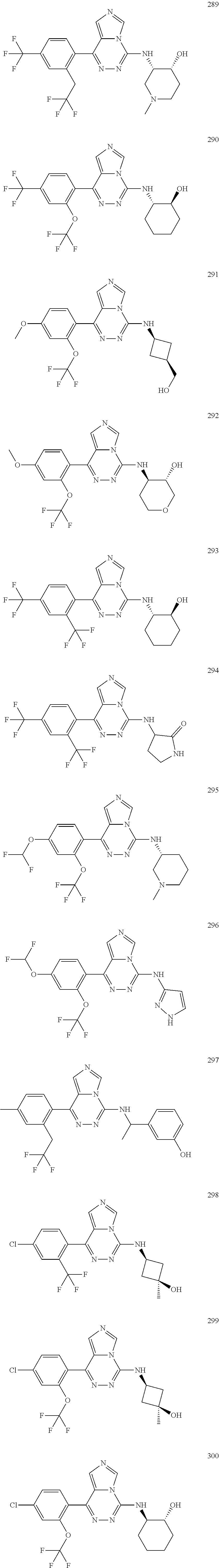 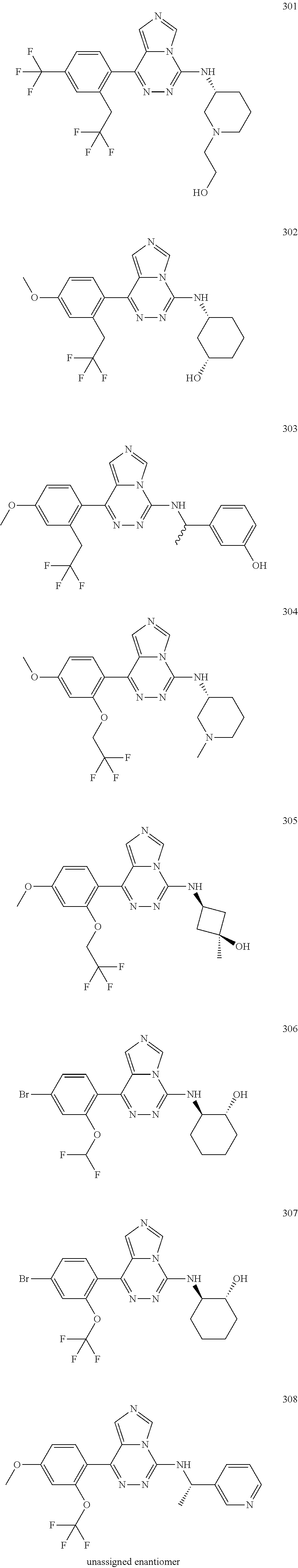 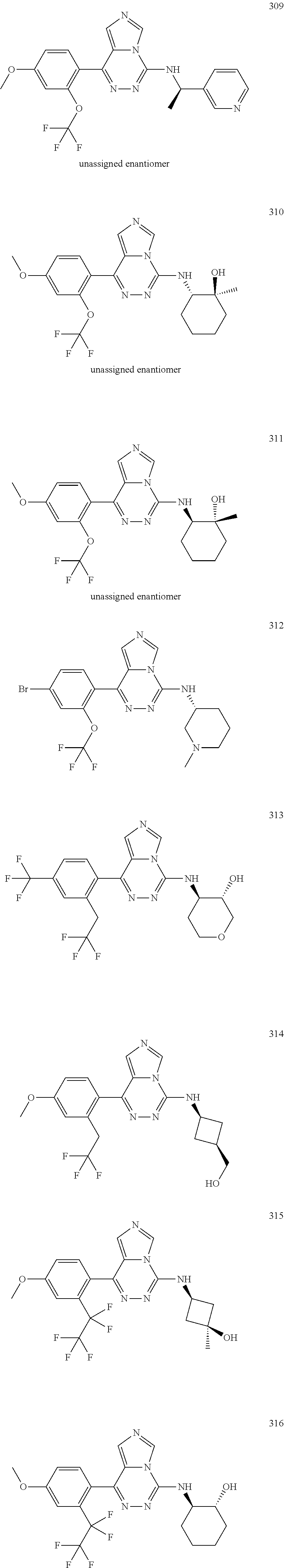 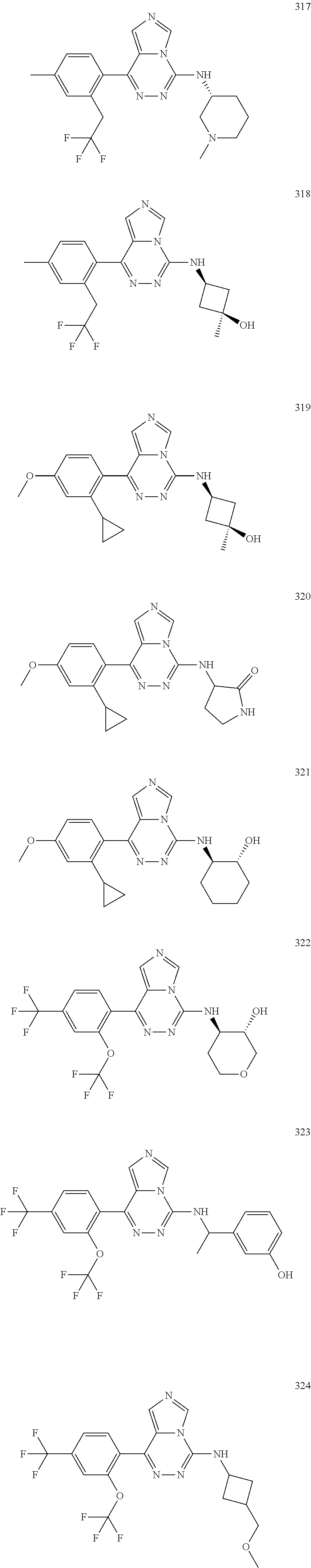 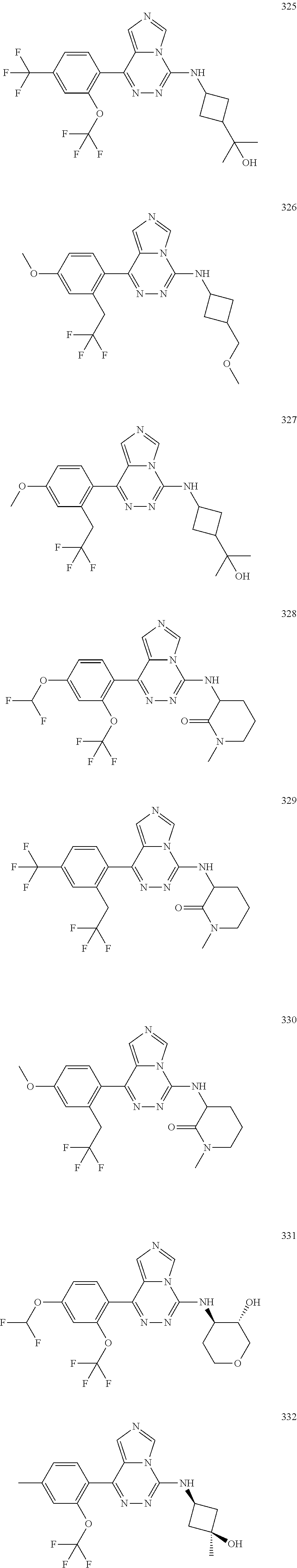 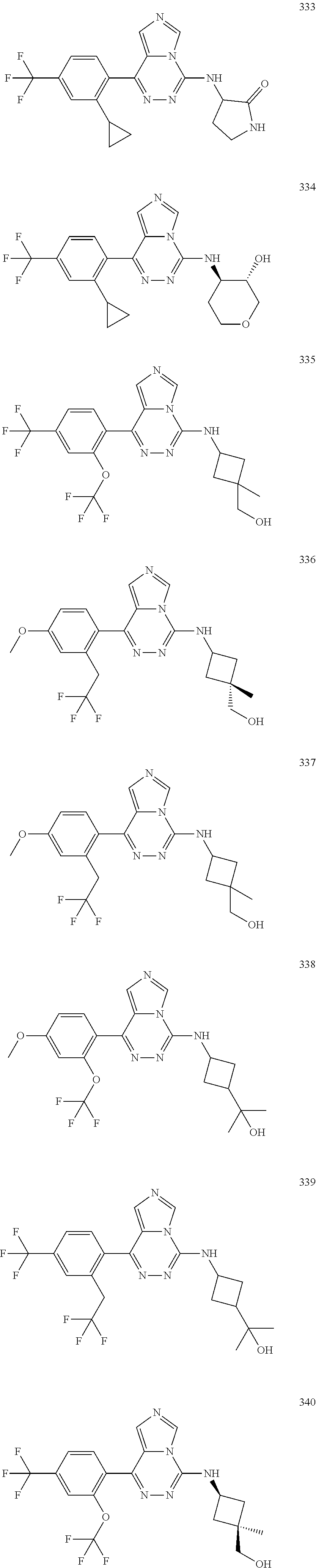 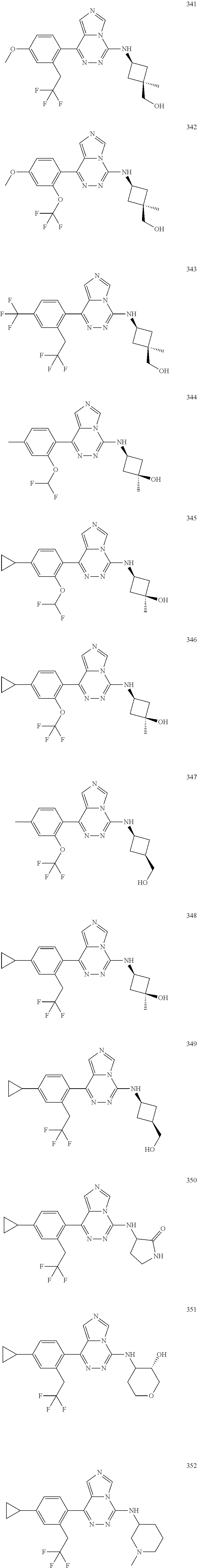 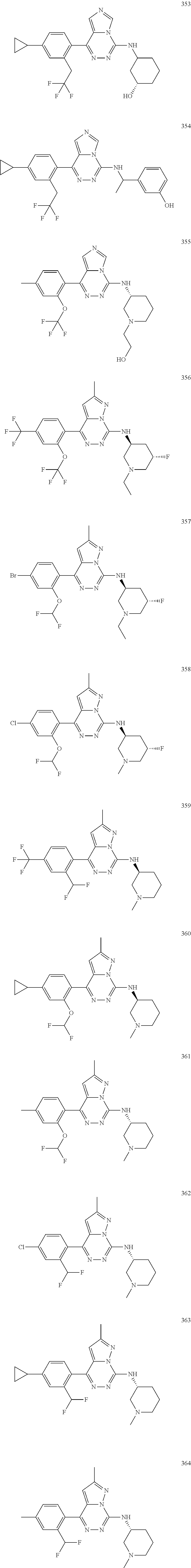 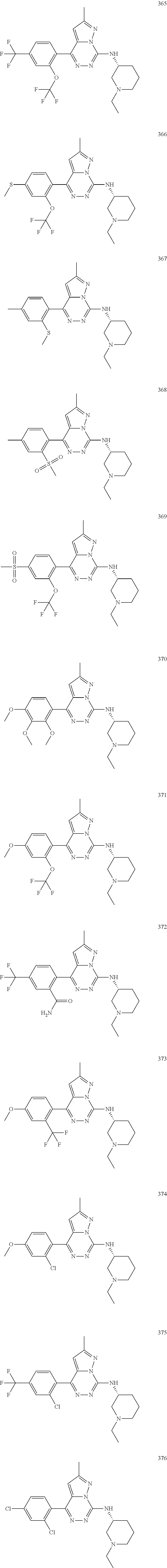 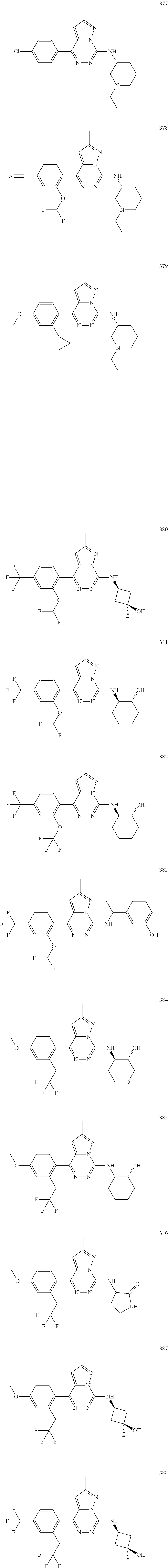 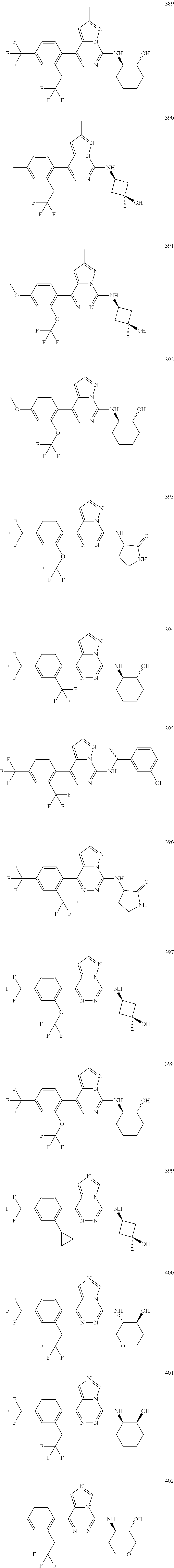
wherein a form of the compound is selected from the group consisting of a pharmaceutically acceptable salt, hydrate, solvate, racemate, enantiomer, diastereomner, steroisomer, tautomer, and isotope enriched form thereof.

2. A compound selected from:

| Compound# | Name |
|---|---|
| 1 | (1s,3s)-3-({1-[4-methoxy-2-(2,2,2-trifluoroethyl)phenyl]pyrrolo[1,2- |
| | d][1,2,4]triazin-4-yl}amino)-1-methylcyclobutan-1-ol |
| 2 | N-[(3R)-1-methylpiperidin-3-yl]-1-[2-(2,2,2-trifluoroethyl)-4- |
| | (trifluoromethyl)phenyl]imidazo[1,5-d][1,2,4]triazin-4-amine |
| 3 | (1s,3s)-1-methyl-3-({2-methyl-4-[2-(trifluoromethoxy)-4- |
| | (trifluoromethyl)phenyl]pyrazolo[1,5-d][1,2,4]triazin-7-yl}amino)cyclobutan- |
| | 1-ol |
| 4 | (1s,3s)-3-({4-[2,4-bis(trifluoromethyl)phenyl]pyrazolo[1,5-d][1,2,4]triazin-7- |
| | yl}amino)-1-methylcyclobutan-1-ol |
| 5 | 1-[2-(difluoromethoxy)-4-(trifluoromethyl)phenyl]-N-[(3R,5R)-1-ethyl-5- |
| | fluoropiperidin-3-yl]-8-fluoropyrrolo[1,2-d][1,2,4]triazin-4-amine |
| 6 | 1-[2,4-bis(trifluoromethyl)phenyl]-N-[(3R)-1-(oxan-3-yl)piperidin-3- |
| | yl]pyrrolo[1,2-d][1,2,4]triazin-4-amine |
| 7 | 1-{1-[2,4-bis(trifluoromethyl)phenyl]pyrrolo[1,2-d][1,2,4]triazin-4-yl}-N,N- |
| | dimethylpiperidin-3-amine |
| 8 | 1-[4-methoxy-2-(trifluoromethoxy)phenyl]-N-[(3R)-1-methylpiperidin-3- |
| | yl]pyrrolo[1,2-d][1,2,4]triazin-4-amine |
| 9 | (1s,3s)-3-({1-[4-methoxy-2-(trifluoromethoxy)phenyl]pyrrolo[1,2- |
| | d][1,2,4]triazin-4-yl}amino)-1-methylcyclobutan-1-ol |
| 10 | 1-[2,4-bis(trifluoromethyl)phenyl]-N-[(3R)-1-(2-methylpropyl)piperidin-3- |
| | yl]pyrrolo[1,2-d][1,2,4]triazin-4-amine |
| 11 | 1-[4-chloro-2-(difluoromethyl)phenyl]-7-methyl-N-[(3R)-1-methylpiperidin-3- |
| | yl]pyrrolo[1,2-d][1,2,4]triazin-4-amine |
| 12 | 1-[2-(difluoromethoxy)-4-methoxyphenyl]-7-methyl-N-[(3R)-1- |
| | methylpiperidin-3-yl]pyrrolo[1,2-d][1,2,4]triazin-4-amine |
| 13 | 1-[4-chloro-2-(difluoromethoxy)phenyl]-7-methyl-N-[(3R)-1-methylpiperidin- |
| | 3-yl]pyrrolo[1,2-d][1,2,4]triazin-4-amine |
| 14 | 1-[4-cyclopropyl-2-(difluoromethyl)phenyl]-7-methyl-N-[(3R)-1- |
| | methylpiperidin-3-yl]pyrrolo[1,2-d][1,2,4]triazin-4-amine |
| 15 | 2-[(3R)-3-({1-[2-(difluoromethoxy)-4-(trifluoromethyl)phenyl]-8- |
| | fluoropyrrolo[1,2-d][1,2,4]triazin-4-yl}amino)piperidin-1-yl]ethan-1-ol |
| 16 | (2S)-1-({1-[2-(difluoromethoxy)-4-(trifluoromethyl)phenyl]-8- |
| | fluoropyrrolo[1,2-d][1,2,4]triazin-4-yl}amino)propan-2-ol |
| 17 | N-[(3R)-1-ethylpiperidin-3-yl]-8-fluoro-1-[4-methoxy-2- |
| | (trifluoromethyl)phenyl]pyrrolo[1,2-d][1,2,4]triazin-4-amine |
| 18 | 2-[(3R)-3-({8-fluoro-1-[4-methoxy-2-(trifluoromethyl)phenyl]pyrrolo[1,2- |
| | d][1,2,4]triazin-4-yl}amino)piperidin-1-yl]ethan-1-ol |
| 19 | 1-[2-fluoro-4-(trifluoromethyl)phenyl]-N-[(3R)-1-methylpiperidin-3- |
| | yl]pyrrolo[1,2-d][1,2,4]triazin-4-amine |
| 20 | N-[(3R)-1-ethylpiperidin-3-yl]-1-[2-fluoro-4- |
| | (trifluoromethyl)phenyl]pyrrolo[1,2-d][1,2,4]triazin-4-amine |
| 21 | 2-[(3R)-3-({1-[2-fluoro-4-(trifluoromethyl)phenyl]pyrrolo[1,2-d][1,2,4]triazin- |
| | 4-yl}amino)piperidin-1-yl]ethan-1-ol |
| 22 | 2-[(3R)-3-({1-[2-(difluoromethoxy)-4-(trifluoromethyl)phenyl]pyrrolo[1,2- |
| | d][1,2,4]triazin-4-yl}amino)piperidin-1-yl]propan-1-ol |
| 23 | 1-[2-(difluoromethyl)-4-methylphenyl]-7-methyl-N-[(3R)-1-methylpiperidin- |
| | 3-yl]pyrrolo[1,2-d][1,2,4]triazin-4-amine |
| 24 | 1-[4-cyclopropyl-2-(difluoromethoxy)phenyl]-7-methyl-N-[(3R)-1- |
| | methylpiperidin-3-yl]pyrrolo[1,2-d][1,2,4]triazin-4-amine |
| 25 | 1-[2-(difluoromethoxy)-4-methylphenyl]-7-methyl-N-[(3R)-1- |
| | methylpiperidin-3-yl]pyrrolo[1,2-d][1,2,4]triazin-4-amine |
| 26 | 1-(4-methoxy-2-methylphenyl)-N-[(3R)-1-methylpiperidin-3-yl]pyrrolo[1,2- |
| | d][1,2,4]triazin-4-amine |
| 27 | 1-(2-cyclopropyl-4-methoxyphenyl)-N-[(3R)-1-methylpiperidin-3- |
| | yl]pyrrolo[1,2-d][1,2,4]triazin-4-amine |
| 28 | 1-[2,4-bis(trifluoromethyl)phenyl]-8-fluoro-N-[(3R)-1-methylpiperidin-3- |
| | yl]pyrrolo[1,2-d][1,2,4]triazin-4-amine |
| 29 | 1-[2,4-bis(trifluoromethyl)phenyl]-N-[(3R)-1-ethylpiperidin-3-yl]-8- |
| | fluoropyrrolo[1,2-d][1,2,4]triazin-4-amine |
| 30 | 2-[(3R)-3-({1-[2,4-bis(trifluoromethyl)phenyl]-8-fluoropyrrolo[1,2- |
| | d][1,2,4]triazin-4-yl}amino)piperidin-1-yl]ethan-1-ol |
| 31 | 1-[2,4-bis(trifluoromethyl)phenyl]-8-fluoro-N-[(3R,5R)-5-fluoro-1- |
| | methylpiperidin-3-yl]pyrrolo[1,2-d][1,2,4]triazin-4-amine |
| 32 | 1-[2,4-bis(trifluoromethyl)phenyl]-N-[(3R,5R)-1-ethyl-5-fluoropiperidin-3-yl]- |
| | 8-fluoropyrrolo[1,2-d][1,2,4]triazin-4-amine |
| 33 | 1-[2-(difluoromethoxy)-4-methylphenyl]-N-[(3R)-1-methylpiperidin-3- |
| | yl]pyrrolo[1,2-d][1,2,4]triazin-4-amine |
| 34 | 1-[2-(difluoromethoxy)-4-methylphenyl]-N-[(3R)-1-ethylpiperidin-3- |
| | yl]pyrrolo[1,2-d][1,2,4]triazin-4-amine |
| 35 | 1-[2-(difluoromethoxy)-4-(trifluoromethyl)phenyl]-N-[(3R)-piperidin-3- |
| | yl]pyrrolo[1,2-d][1,2,4]triazin-4-amine |
| 36 | N-[(3R)-1-methylpiperidin-3-yl]-1-[4-methyl-2- |
| | (trifluoromethoxy)phenyl]pyrrolo[1,2-d][1,2,4]triazin-4-amine |
| 37 | 1-[4-cyclopropyl-2-(trifluoromethoxy)phenyl]-N-[(3R)-1-methylpiperidin-3- |
| | yl]pyrrolo[1,2-d][1,2,4]triazin-4-amine |
| 38 | (1s,3s)-3-({1-[2,4-bis(trifluoromethyl)phenyl]-8-fluoropyrrolo[1,2- |
| | d][1,2,4]triazin-4-yl}amino)-1-methylcyclobutan-1-ol |
| 39 | 8-fluoro-1-[4-methoxy-2-(trifluoromethoxy)phenyl]-N-[(3R)-1- |
| | methylpiperidin-3-yl]pyrrolo[1,2-d][1,2,4]triazin-4-amine |
| 40 | N-[(3R)-1-methylpiperidin-3-yl]-1-[2-(2,2,2-trifluoroethyl)-4- |
| | (trifluoromethyl)phenyl]pyrrolo[1,2-d][1,2,4]triazin-4-amine |
| 41 | (1s,3s)-1-methyl-3-({1-[2-(2,2,2-trifluoroethyl)-4- |
| | (trifluoromethyl)phenyl]pyrrolo[1,2-d][1,2,4]triazin-4-yl}amino)cyclobutan-1- |
| | ol |
| 42 | 1-[4-ethyl-2-(trifluoromethoxy)phenyl]-N-[(3R)-1-methylpiperidin-3- |
| | yl]pyrrolo[1,2-d][1,2,4]triazin-4-amine |
| 43 | 1-[4-ethoxy-2-(trifluoromethoxy)phenyl]-N-[(3R)-1-methylpiperidin-3- |
| | yl]pyrrolo[1,2-d][1,2,4]triazin-4-amine |
| 44 | 1-[4-(difluoromethoxy)-2-(trifluoromethoxy)phenyl]-N-[(3R)-1- |
| | methylpiperidin-3-yl]pyrrolo[1,2-d][1,2,4]triazin-4-amine |
| 45 | 2-[(3R)-3-({1-[2-(difluoromethoxy)-4-methylphenyl]pyrrolo[1,2- |
| | d][1,2,4]triazin-4-yl}amino)piperidin-1-yl]ethan-1-ol |
| 46 | (3S,4S)-3-({1-[2-(difluoromethoxy)-4-(trifluoromethyl)phenyl]pyrrolo[1,2- |
| | d][1,2,4]triazin-4-yl}amino)-1-ethylpiperidin-4-ol |
| 47 | 1-(2-bromo-4-methoxyphenyl)-N-[(3R)-1-methylpiperidin-3-yl]pyrrolo[1,2- |
| | d][1,2,4]triazin-4-amine |
| 48 | (3S,4R)-3-({1-[2-(difluoromethoxy)-4-(trifluoromethyl)phenyl]pyrrolo[1,2- |
| | d][1,2,4]triazin-4-yl}amino)-1-ethylpiperidin-4-ol |
| 49 | 1-[2-(difluoromethoxy)-6-methyl-4-(trifluoromethyl)phenyl]-N-[(3R)-1- |
| | methylpiperidin-3-yl]pyrrolo[1,2-d][1,2,4]triazin-4-amine |
| 50 | N-[(3R)-1-ethylpiperidin-3-yl]-8-fluoro-1-[4-methoxy-2- |
| | (trifluoromethoxy)phenyl]pyrrolo[1,2-d][1,2,4]triazin-4-amine |
| 51 | 2-[(3R)-3-({8-fluoro-1-[4-methoxy-2-(trifluoromethoxy)phenyl]pyrrolo[1,2- |
| | d][1,2,4]triazin-4-yl}amino)piperidin-1-yl]ethan-1-ol |
| 52 | 8-fluoro-N-[(3R,SR)-5-fluoro-1-methylpiperidin-3-yl]-1-[4-methoxy-2- |
| | (trifluoromethoxy)phenyl]pyrrolo[1,2-d][1,2,4]triazin-4-amine |
| 53 | N-[(3R,5R)-1-ethyl-5-fluoropiperidin-3-yl]-8-fluoro-1-[4-methoxy-2- |
| | (trifluoromethoxy)phenyl]pyrrolo[1,2-d][1,2,4]triazin-4-amine |
| 54 | (1s,3s)-3-({8-fluoro-1-[4-methoxy-2-(trifluoromethoxy)phenyl]pyrrolo[1,2- |
| | d][1,2,4]triazin-4-yl}amino)-1-methylcyclobutan-1-ol |
| 55 | 1-[2-(difluoromethoxy)-4-methoxyphenyl]-8-fluoro-N-[(3R)-1- |
| | methylpiperidin-3-yl]pyrrolo[1,2-d][1,2,4]triazin-4-amine |
| 56 | 1-[2-(difluoromethoxy)-4-methoxyphenyl]-N-[(3R)-1-ethylpiperidin-3-yl]-8- |
| | fluoropyrrolo[1,2-d][1,2,4]triazin-4-amine |
| 57 | 2-[(3R)-3-({1-[2-(difluoromethoxy)-4-methoxyphenyl]-8-fluoropyrrolo[1,2- |
| | d][1,2,4]triazin-4-yl}amino)piperidin-1-yl]ethan-1-ol |
| 58 | (1s,3s)-3-({1-[2-(difluoromethoxy)-4-methoxyphenyl]-8-fluoropyrrolo[1,2- |
| | d][1,2,4]triazin-4-yl}amino)-1-methylcyclobutan-1-ol |
| 59 | N-[(3R)-1-ethylpiperidin-3-yl]-1-[4-methoxy-2- |
| | (trifluoromethoxy)phenyl]pyrrolo[1,2-d][1,2,4]triazin-4-amine |
| 60 | N-[(3R,5R)-5-fluoro-1-methylpiperidin-3-yl]-1-[4-methoxy-2- |
| | (trifluoromethoxy)phenyl]pyrrolo[1,2-d][1,2,4]triazin-4-amine |
| 61 | 2-[(3R)-3-({1-[4-methoxy-2-(trifluoromethoxy)phenyl]pyrrolo[1,2- |
| | d][1,2,4]triazin-4-yl}amino)piperidin-1-yl]ethan-1-ol |
| 62 | (1S,3R)-3-({1-[4-methoxy-2-(trifluoromethoxy)phenyl]pyrrolo[1,2- |
| | d][1,2,4]triazin-4-yl}amino)cyclohexan-1-ol |
| 63 | (2R)-1-({1-[2-(difluoromethoxy)-4-(trifluoromethyl)phenyl]pyrrolo[1,2- |
| | d][1,2,4]triazin-4-yl}amino)propan-2-ol |
| 64 | 2-({1-[4-methoxy-2-(trifluoromethoxy)phenyl]pyrrolo[1,2-d][1,2,4]triazin-4- |
| | yl}amino)ethan-1-ol |
| 65 | (2S)-1-({1-[4-methoxy-2-(trifluoromethoxy)phenyl]pyrrolo[1,2- |
| | d][1,2,4]triazin-4-yl}amino)propan-2-ol |
| 66 | 1-({1-[4-methoxy-2-(trifluoromethoxy)phenyl]pyrrolo[1,2-d][1,2,4]triazin-4- |
| | yl}amino)-2-methylpropan-2-ol |
| 67 | 1-[4-methoxy-2-(trifluoromethoxy)phenyl]-N-(oxan-4-yl)pyrrolo[1,2- |
| | d][1,2,4]triazin-4-amine |
| 68 | 1-[4-methoxy-2-(trifluoromethoxy)phenyl]-N-[(3R)-oxolan-3-yl]pyrrolo[1,2- |
| | d][1,2,4]triazin-4-amine |
| 69 | 1-[4-methoxy-2-(trifluoromethoxy)phenyl]-N-[(1-methyl-1H-imidazol-2- |
| | yl)methyl]pyrrolo[1,2-d][1,2,4]triazin-4-amine |
| 70 | (2R)-1-({1-[4-methoxy-2-(trifluoromethoxy)phenyl]pyrrolo[1,2- |
| | d][1,2,4]triazin-4-yl}amino)propan-2-ol |
| 71 | (1s,3s)-3-({1-[4-methoxy-2-(trifluoromethyl)phenyl]pyrrolo[1,2- |
| | d][1,2,4]triazin-4-yl}amino)-1-methylcyclobutan-1-ol |
| 72 | (1s,3s)-3-{[1-(2-chloro-4-methoxyphenyl)pyrrolo[1,2-d][1,2,4]triazin-4- |
| | yl]amino}-1-methylcyclobutan-1-ol |
| 73 | 1-[4-methoxy-2-(trifluoromethoxy)phenyl]-N-{[(2R)-oxolan-2- |
| | yl]methyl}pyrrolo[1,2-d][1,2,4]triazin-4-amine |
| 74 | 1-[4-methoxy-2-(trifluoromethoxy)phenyl]-N-{[(2S)-1-methylpyrrolidin-2- |
| | yl]methyl}pyrrolo[1,2-d][1,2,4]triazin-4-amine |
| 75 | 1-(2-cyclopropyl-6-fluoro-4-methoxyphenyl)-N-[(3R)-1-methylpiperidin-3- |
| | yl]pyrrolo[1,2-d][1,2,4]triazin-4-amine |
| 76 | 1-[4-methoxy-2-(trifluoromethoxy)phenyl]-N-{[(2S)-oxolan-2- |
| | yl]methyl}pyrrolo[1,2-d][1,2,4]triazin-4-amine |
| 77 | 3-({1-[2-(2,2,2-trifluoroethyl)-4-(trifluoromethyl)phenyl]pyrrolo[1,2- |
| | d][1,2,4]triazin-4-yl}amino)pyrrolidin-2-one |
| 78 | (1S,2S)-2-({1-[2-(difluoromethoxy)-4-(trifluoromethyl)phenyl]pyrrolo[1,2- |
| | d][1,2,4]triazin-4-yl}amino)cyclohexan-1-ol |
| 79 | (1R,2S)-2-({1-[2-(difluoromethoxy)-4-(trifluoromethyl)phenyl]pyrrolo[1,2- |
| | d][1,2,4]triazin-4-yl}amino)cyclohexan-1-ol |
| 80 | (1S,2S)-2-({1-[2-(difluoromethoxy)-4-methylphenyl]pyrrolo[1,2- |
| | d][1,2,4]triazin-4-yl}amino)cyclohexan-1-ol |
| 81 | (1S,2S)-2-({1-[4-methoxy-2-(trifluoromethoxy)phenyl]pyrrolo[1,2- |
| | d][1,2,4]triazin-4-yl}amino)cyclohexan-1-ol |
| 82 | 1-(2-chloro-4-methoxyphenyl)-N-[(3R)-1-ethylpiperidin-3-yl]-8- |
| | fluoropyrrolo[1,2-d][1,2,4]triazin-4-amine |
| 83 | 2-[(3R)-3-{[1-(2-chloro-4-methoxyphenyl)-8-fluoropyrrolo[1,2- |
| | d][1,2,4]triazin-4-yl]amino}piperidin-1-yl]ethan-1-ol |
| 84 | (1s,3s)-3-{[1-(2-chloro-4-methoxyphenyl)-8-fluoropyrrolo[1,2- |
| | d][1,2,4]triazin-4-yl]amino}-1-methylcyclobutan-1-ol |
| 85 | 1-[4-(difluoromethoxy)-2-(trifluoromethoxy)phenyl]-N-[(3R)-1- |
| | ethylpiperidin-3-yl]pyrrolo[1,2-d][1,2,4]triazin-4-amine |
| 86 | 2-[(3R)-3-({1-[4-(difluoromethoxy)-2-(trifluoromethoxy)phenyl]pyrrolo[1,2- |
| | d][1,2,4]triazin-4-yl}amino)piperidin-1-yl]ethan-1-ol |
| 87 | 1-[4-(difluoromethoxy)-2-(trifluoromethoxy)phenyl]-N-[(3R,5R)-5-fluoro-1- |
| | methylpiperidin-3-yl]pyrrolo[1,2-d][1,2,4]triazin-4-amine |
| 88 | (1s,3s)-3-({1-[4-(difluoromethoxy)-2-(trifluoromethoxy)phenyl]pyrrolo[1,2- |
| | d][1,2,4]triazin-4-yl}amino)-1-methylcyclobutan-1-ol |
| 89 | 3-[1-({1-[2-(difluoromethoxy)-4-(trifluoromethyl)phenyl]pyrrolo[1,2- |
| | d][1,2,4]triazin-4-yl}amino)ethyl]phenol |
| 90 | (1R,2R)-2-({1-[2-(2,2,2-trifluoroethyl)-4-(trifluoromethyl)phenyl]pyrrolo[1,2- |
| | d][1,2,4]triazin-4-yl}amino)cyclohexan-1-ol |
| 91 | 1-[4-methoxy-2-(trifluoromethoxy)phenyl]-N-[(1,3-oxazol-2- |
| | yl)methyl]pyrrolo[1,2-d][1,2,4]triazin-4-amine |
| 92 | 1-[4-methoxy-2-(trifluoromethoxy)phenyl]-N-[(4-methyl-4H-1,2,4-triazol-3- |
| | yl)methyl]pyrrolo[1,2-d][1,2,4]triazin-4-amine |
| 93 | 1-[4-methoxy-2-(trifluoromethoxy)phenyl]-N-[(1-methyl-1H-1,2,4-triazol-5- |
| | yl)methyl]pyrrolo[1,2-d][1,2,4]triazin-4-amine |
| 94 | [3-({1-[4-methoxy-2-(trifluoromethoxy)phenyl]pyrrolo[1,2-d][1,2,4]triazin-4- |
| | yl}amino)bicyclo[1.1.1]pentan-1-yl]methanol |
| 95 | 1-[3-(difluoromethoxy)-4-(4-{[(3R)-1-methylpiperidin-3- |
| | yl]amino}pyrrolo[1,2-d][1,2,4]triazin-1-yl)phenyl]propan-1-one |
| 96 | 3-[1-({1-[2-(2,2,2-trifluoroethyl)-4-(trifluoromethyl)phenyl]pyrrolo[1,2- |
| | d][1,2,4]triazin-4-yl}amino)ethyl]phenol |
| 97 | N-(1H-pyrazol-3-yl)-1-[2-(2,2,2-trifluoroethyl)-4- |
| | (trifluoromethyl)phenyl]pyrrolo[1,2-d][1,2,4]triazin-4-amine |
| 98 | (3S,5R)-5-({1-[2-(difluoromethoxy)-4-(trifluoromethyl)phenyl]pyrrolo[1,2- |
| | d][1,2,4]triazin-4-yl}amino)-1-methylpiperidin-3-ol |
| 99 | (1S,2S)-2-({1-[2,4-bis(trifluoromethyl)phenyl]pyrrolo[1,2-d][1,2,4]triazin-4- |
| | yl}amino)cyclohexan-1-ol |
| 100 | 3-[1-({1-[2-(difluoromethoxy)-4-methylphenyl]pyrrolo[1,2-d][1,2,4]triazin-4- |
| | yl}amino)ethyl]phenol |
| 101 | 3-[({1-[2-(difluoromethoxy)-4-(trifluoromethyl)phenyl]pyrrolo[1,2- |
| | d][1,2,4]triazin-4-yl}amino)methyl]phenol |
| 102 | 3-[(1R)-1-({1-[2-(difluoromethoxy)-4-(trifluoromethyl)phenyl]pyrrolo[1,2- |
| | d][1,2,4]triazin-4-yl}amino)ethyl]phenol OR 3-[(1S)-1-({1-[2- |
| | (difluoromethoxy)-4-(trifluoromethyl)phenyl]pyrrolo[1,2-d][1,2,4]triazin-4- |
| | yl}amino)ethyl]phenol |
| 103 | 3-[(1S)-1-({1-[2-(difluoromethoxy)-4-(trifluoromethyl)phenyl]pyrrolo[1,2- |
| | d][1,2,4]triazin-4-yl}amino)ethyl]phenol OR 3-[(1R)-1-({1-[2- |
| | (difluoromethoxy)-4-(trifluoromethyl)phenyl]pyrrolo[1,2-d][1,2,4]triazin-4- |
| | yl}amino)ethyl]phenol |
| 104 | (1s,3s)-3-{[1-(2-cyclopropyl-4-methoxyphenyl)pyrrolo[1,2-d][1,2,4]triazin-4- |
| | yl]amino}-1-methylcyclobutan-1-ol |
| 105 | 1-(2-cyclopropyl-4-methoxyphenyl)-N-(1H-pyrazol-3-yl)pyrrolo[1,2- |
| | d][1,2,4]triazin-4-amine |
| 106 | (1S,3R)-3-{[1-(2-cyclopropyl-4-methoxyphenyl)pyrrolo[1,2-d][1,2,4]triazin-4- |
| | yl]amino}cyclohexan-1-ol |
| 107 | (1s,3s)-3-({1-[2,4-bis(trifluoromethyl)phenyl]-7-methylpyrrolo[1,2- |
| | d][1,2,4]triazin-4-yl}amino)-1-methylcyclobutan-1-ol |
| 108 | (1s,3s)-3-({1-[4-methoxy-2-(trifluoromethoxy)phenyl]-7-methylpyrrolo[1,2- |
| | d][1,2,4]triazin-4-yl}amino)-1-methylcyclobutan-1-ol |
| 109 | 1-[2,4-bis(trifluoromethyl)phenyl]-7-methyl-N-[(3R)-1-methylpiperidin-3- |
| | yl]pyrrolo[1,2-d][1,2,4]triazin-4-amine |
| 110 | (1R,2R)-2-({1-[2-(difluoromethoxy)-4-(trifluoromethyl)phenyl]pyrrolo[1,2- |
| | d][1,2,4]triazin-4-yl}amino)cyclohexan-1-ol |
| 111 | (1S,2S)-2-({1-[2-(2,2,2-trifluoroethyl)-4-(trifluoromethyl)phenyl]pyrrolo[1,2- |
| | d][1,2,4]triazin-4-yl}amino)cyclohexan-1-ol |
| 112 | (3S,4R)-4-({1-[2-(2,2,2-trifluoroethyl)-4-(trifluoromethyl)phenyl]pyrrolo[1,2- |
| | d][1,2,4]triazin-4-yl}amino)oxan-3-ol |
| 113 | (3R,4S)-4-({1-[2-(2,2,2-trifluoroethyl)-4-(trifluoromethyl)phenyl]pyrrolo[1,2- |
| | d][1,2,4]triazin-4-yl}amino)oxan-3-ol |
| 114 | (3R,4R)-4-({1-[2-(2,2,2-trifluoroethyl)-4-(trifluoromethyl)phenyl]pyrrolo[1,2- |
| | d][1,2,4]triazin-4-yl}amino)oxan-3-ol |
| 115 | (3S,4S)-4-({1-[2-(2,2,2-trifluoroethyl)-4-(trifluoromethyl)phenyl]pyrrolo[1,2- |
| | d][1,2,4]triazin-4-yl}amino)oxan-3-ol |
| 116 | 3-({8-fluoro-1-[4-methoxy-2-(trifluoromethyl)phenyl]pyrrolo[1,2- |
| | d][1,2,4]triazin-4-yl}amino)pyrrolidin-2-one |
| 117 | 8-fluoro-1-[4-methoxy-2-(trifluoromethyl)phenyl]-N-(1H-pyrazol-3- |
| | yl)pyrrolo[1,2-d][1,2,4]triazin-4-amine |
| 118 | 1-(2-fluoro-4-methoxy-6-methylphenyl)-N-[(3R)-1-methylpiperidin-3- |
| | yl]pyrrolo[1,2-d][1,2,4]triazin-4-amine |
| 119 | N-[(3R)-1-ethylpiperidin-3-yl]-1-[2-(trifluoromethoxy)-4- |
| | (trifluoromethyl)phenyl]pyrrolo[1,2-d][1,2,4]triazin-4-amine |
| 120 | 2-[(3R)-3-({1-[2-(trifluoromethoxy)-4-(trifluoromethyl)phenyl]pyrrolo[1,2- |
| | d][1,2,4]triazin-4-yl}amino)piperidin-1-yl]ethan-1-ol |
| 121 | (1S,2S)-2-({1-[2-(trifluoromethoxy)-4-(trifluoromethyl)phenyl]pyrrolo[1,2- |
| | d][1,2,4]triazin-4-yl}amino)cyclohexan-1-ol |
| 122 | (1S,3R)-3-({1-[2-(trifluoromethoxy)-4-(trifluoromethyl)phenyl]pyrrolo[1,2- |
| | d][1,2,4]triazin-4-yl}amino)cyclohexan-1-ol |
| 123 | (1s,3s)-1-methyl-3-({1-[2-(trifluoromethoxy)-4- |
| | (trifluoromethyl)phenyl]pyrrolo[1,2-d][1,2,4]triazin-4-yl}amino)cyclobutan-1- |
| | ol |
| 124 | 3-({1-[2-(trifluoromethoxy)-4-(trifluoromethyl)phenyl]pyrrolo[1,2- |
| | d][1,2,4]triazin-4-yl}amino)pyrrolidin-2-one |
| 125 | 1-[4-methoxy-2-(trifluoromethoxy)phenyl]-N-[(1-methyl-1H-tetrazol-5- |
| | yl)methyl]pyrrolo[1,2-d][1,2,4]triazin-4-amine |
| 126 | (1S,2R)-2-({1-[2-(2,2,2-trifluoroethyl)-4-(trifluoromethyl)phenyl]pyrrolo[1,2- |
| | d][1,2,4]triazin-4-yl}amino)cycloheptan-1-ol AND (1R,2S)-2-({1-[2-(2,2,2- |
| | trifluoroethyl)-4-(trifluoromethyl)phenyl]pyrrolo[1,2-d][1,2,4]triazin-4- |
| | yl}amino)cycloheptan-1-ol |
| 127 | (1R,2R)-2-({1-[2-(2,2,2-trifluoroethyl)-4-(trifluoromethyl)phenyl]pyrrolo[1,2- |
| | d][1,2,4]triazin-4-yl}amino)cycloheptan-1-ol AND (1S,2S)-2-({1-[2-(2,2,2- |
| | trifluoroethyl)-4-(trifluoromethyl)phenyl]pyrrolo[1,2-d][1,2,4]triazin-4- |
| | yl}amino)cycloheptan-1-ol |
| 128 | (1S,3R)-3-({1-[2-(2,2,2-trifluoroethyl)-4-(trifluoromethyl)phenyl]pyrrolo[1,2- |
| | d][1,2,4]triazin-4-yl}amino)cycloheptan-1-ol AND (1R,3S)-3-({1-[2-(2,2,2- |
| | trifluoroethyl)-4-(trifluoromethyl)phenyl]pyrrolo[1,2-d][1,2,4]triazin-4- |
| | yl}amino)cycloheptan-1-ol |
| 129 | (1R,3R)-3-({1-[2-(2,2,2-trifluoroethyl)-4-(trifluoromethyl)phenyl]pyrrolo[1,2- |
| | d][1,2,4]triazin-4-yl}amino)cycloheptan-1-ol AND (1S,3S)-3-({1-[2-(2,2,2- |
| | trifluoroethyl)-4-(trifluoromethyl)phenyl]pyrrolo[1,2-d][1,2,4]triazin-4- |
| | yl}amino)cycloheptan-1-ol |
| 130 | (1s,3s)-3-({8-fluoro-1-[4-methoxy-2-(trifluoromethyl)phenyl]pyrrolo[1,2- |
| | d][1,2,4]triazin-4-yl}amino)-1-methylcyclobutan-1-ol |
| 131 | 1-[4-methoxy-2-(trifluoromethoxy)phenyl]-7-methyl-N-[(3R)-1- |
| | methylpiperidin-3-yl]pyrrolo[1,2-d][1,2,4]triazin-4-amine |
| 132 | (1S,2S)-2-({1-[2,4-bis(trifluoromethyl)phenyl]-7-methylpyrrolo[1,2- |
| | d][1,2,4]triazin-4-yl}amino)cyclohexan-1-ol |
| 133 | (1S,2S)-2-({1-[4-methoxy-2-(trifluoromethoxy)phenyl]-7-methylpyrrolo[1,2- |
| | d][1,2,4]triazin-4-yl}amino)cyclohexan-1-ol |
| 134 | (3S,4R)-3-({1-[2,4-bis(trifluoromethyl)phenyl]-7-methylpyrrolo[1,2- |
| | d][1,2,4]triazin-4-yl}amino)-1-ethylpiperidin-4-ol |
| 135 | (3S,4R)-1-ethyl-3-({1-[4-methoxy-2-(trifluoromethoxy)phenyl]-7- |
| | methylpyrrolo[1,2-d][1,2,4]triazin-4-yl}amino)piperidin-4-ol |
| 136 | (1R,2R)-2-({1-[2-(difluoromethoxy)-4-methylphenyl]pyrrolo[1,2- |
| | d][1,2,4]triazin-4-yl}amino)cyclohexan-1-ol |
| 137 | (1S,2R)-2-({1-[2-(difluoromethoxy)-4-methylphenyl]pyrrolo[1,2- |
| | d][1,2,4]triazin-4-yl}amino)cyclohexan-1-ol |
| 138 | (1R,2R)-2-({1-[4-methoxy-2-(2,2,2-trifluoroethyl)phenyl]pyrrolo[1,2- |
| | d][1,2,4]triazin-4-yl}amino)cyclohexan-1-ol |
| 139 | 3-({1-[4-methoxy-2-(2,2,2-trifluoroethyl)phenyl]pyrrolo[1,2-d][1,2,4]triazin- |
| | 4-yl}amino)pyrrolidin-2-one |
| 140 | N-[(3R)-1-methylpiperidin-3-yl]-1-[2-(trifluoromethoxy)-4- |
| | (trifluoromethyl)phenyl]pyrrolo[1,2-d][1,2,4]triazin-4-amine |
| 141 | [(1s,3s)-3-({1-[2-(trifluoromethoxy)-4-(trifluoromethyl)phenyl]pyrrolo[1,2- |
| | d][1,2,4]triazin-4-yl}amino)cyclobutyl]methanol |
| 142 | 3-[1-({1-[2-(trifluoromethoxy)-4-(trifluoromethyl)phenyl]pyrrolo[1,2- |
| | d][1,2,4]triazin-4-yl}amino)ethyl]phenol |
| 143 | 2-[(3R)-3-({1-[2-(cyclopropyloxy)-4-methoxyphenyl]pyrrolo[1,2- |
| | d][1,2,4]triazin-4-yl}amino)piperidin-1-yl]ethan-1-ol |
| 144 | (3S,4R)-4-({1-[4-methoxy-2-(2,2,2-trifluoroethyl)phenyl]pyrrolo[1,2- |
| | d][1,2,4]triazin-4-yl}amino)oxan-3-ol |
| 145 | 1-[4-methoxy-2-(2,2,2-trifluoroethyl)phenyl]-N-[(3R)-1-methylpiperidin-3- |
| | yl]pyrrolo[1,2-d][1,2,4]triazin-4-amine |
| 146 | N-{[(3S)-1-methylpiperidin-3-yl]methyl}-1-[2-(2,2,2-trifluoroethyl)-4- |
| | (trifluoromethyl)phenyl]pyrrolo[1,2-d][1,2,4]triazin-4-amine |
| 147 | N-{[(3R)-1-methylpiperidin-3-yl]methyl}-1-[2-(2,2,2-trifluoroethyl)-4- |
| | (trifluoromethyl)phenyl]pyrrolo[1,2-d][1,2,4]triazin-4-amine |
| 148 | 8-fluoro-N-[1-(3-methoxyphenyl)ethyl]-1-[4-methoxy-2- |
| | (trifluoromethyl)phenyl]pyrrolo[1,2-d][1,2,4]triazin-4-amine |
| 149 | 3-[1-({8-fluoro-1-[4-methoxy-2-(trifluoromethyl)phenyl]pyrrolo[1,2- |
| | d][1,2,4]triazin-4-yl}amino)ethyl]phenol |
| 150 | N-(1H-pyrazol-3-yl)-1-[2-(trifluoromethoxy)-4- |
| | (trifluoromethyl)phenyl]pyrrolo[1,2-d][1,2,4]triazin-4-amine |
| 151 | (1R,2R)-2-({1-[2-(trifluoromethoxy)-4-(trifluoromethyl)phenyl]pyrrolo[1,2- |
| | d][1,2,4]triazin-4-yl}amino)cyclohexan-1-ol |
| 152 | 1-[4-(difluoromethoxy)-2-(trifluoromethoxy)phenyl]-N-[(3R)-oxan-3- |
| | yl]pyrrolo[1,2-d][1,2,4]triazin-4-amine |
| 153 | (1S,3R)-3-({1-[4-(difluoromethoxy)-2-(trifluoromethoxy)phenyl]pyrrolo[1,2- |
| | d][1,2,4]triazin-4-yl}amino)cyclohexan-1-ol |
| 154 | 3-({1-[4-(difluoromethoxy)-2-(trifluoromethoxy)phenyl]pyrrolo[1,2- |
| | d][1,2,4]triazin-4-yl}amino)pyrrolidin-2-one |
| 155 | 1-[4-(difluoromethoxy)-2-(trifluoromethoxy)phenyl]-N-(1H-pyrazol-3- |
| | yl)pyrrolo[1,2-d][1,2,4]triazin-4-amine |
| 156 | (1R,2R)-2-({1-[4-methyl-2-(2,2,2-trifluoroethyl)phenyl]pyrrolo[1,2- |
| | d][1,2,4]triazin-4-yl}amino)cyclohexan-1-ol |
| 157 | 3-({1-[4-methyl-2-(2,2,2-trifluoroethyl)phenyl]pyrrolo[1,2-d][1,2,4]triazin-4- |
| | yl}amino)pyrrolidin-2-one |
| 158 | (1s,3s)-1-methyl-3-({1-[4-methyl-2-(2,2,2-trifluoroethyl)phenyl]pyrrolo[1,2- |
| | d][1,2,4]triazin-4-yl}amino)cyclobutan-1-ol |
| 159 | N-[(3R)-1-methylpiperidin-3-yl]-1-[4-methyl-2-(2,2,2- |
| | trifluoroethyl)phenyl]pyrrolo[1,2-d][1,2,4]triazin-4-amine |
| 160 | (1R,2R)-2-({1-[2,4-bis(trifluoromethyl)phenyl]-7-methylpyrrolo[1,2- |
| | d][1,2,4]triazin-4-yl}amino)cyclohexan-1-ol |
| 161 | 3-({1-[2,4-bis(trifluoromethyl)phenyl]-7-methylpyrrolo[1,2-d][1,2,4]triazin-4- |
| | yl}amino)pyrrolidin-2-one |
| 162 | 3-({1-[4-methoxy-2-(trifluoromethoxy)phenyl]-7-methylpyrrolo[1,2- |
| | d][1,2,4]triazin-4-yl}amino)pyrrolidin-2-one |
| 163 | (1R,2R)-2-({1-[4-methoxy-2-(trifluoromethoxy)phenyl]-7-methylpyrrolo[1,2- |
| | d][1,2,4]triazin-4-yl}amino)cyclohexan-1-ol |
| 164 | (3S,4R)-4-({1-[2,4-bis(trifluoromethyl)phenyl]-7-methylpyrrolo[1,2- |
| | d][1,2,4]triazin-4-yl}amino)oxan-3-ol |
| 165 | (3S,4R)-4-({1-[4-methoxy-2-(trifluoromethoxy)phenyl]-7-methylpyrrolo[1,2- |
| | d][1,2,4]triazin-4-yl}amino)oxan-3-ol |
| 166 | 1-[2-(difluoromethoxy)-4-methoxyphenyl]-N-[(3R)-1-ethylpiperidin-3- |
| | yl]pyrrolo[1,2-d][1,2,4]triazin-4-amine |
| 167 | (3S,4R)-4-({1-[2-(difluoromethoxy)-4-methoxyphenyl]pyrrolo[1,2- |
| | d][1,2,4]triazin-4-yl}amino)oxan-3-ol |
| 168 | (1R,2R)-2-({1-[4-(difluoromethoxy)-2-(trifluoromethoxy)phenyl]pyrrolo[1,2- |
| | d][1,2,4]triazin-4-yl}amino)cyclohexan-1-ol |
| 169 | N-[(3R)-1-ethylpiperidin-3-yl]-1-[2-(fluoromethoxy)-4- |
| | (trifluoromethyl)phenyl]pyrrolo[1,2-d][1,2,4]triazin-4-amine |
| 170 | (3S,4R)-3-({1-[4-(difluoromethoxy)-2-(trifluoromethoxy)phenyl]pyrrolo[1,2- |
| | d][1,2,4]triazin-4-yl}amino)-1-methylpiperidin-4-ol |
| 171 | (3S,4R)-3-({1-[4-(difluoromethoxy)-2-(trifluoromethoxy)phenyl]pyrrolo[1,2- |
| | d][1,2,4]triazin-4-yl}amino)-1-ethylpiperidin-4-ol |
| 172 | 3-[1-({1-[4-(difluoromethoxy)-2-(trifluoromethoxy)phenyl]pyrrolo[1,2- |
| | d][1,2,4]triazin-4-yl}amino)ethyl]phenol |
| 173 | 2-[(3R)-3-({1-[2-(fluoromethoxy)-4-(trifluoromethyl)phenyl]pyrrolo[1,2- |
| | d][1,2,4]triazin-4-yl}amino)piperidin-1-yl]ethan-1-ol |
| 174 | 3-({1-[2-(difluoromethoxy)-4-methoxyphenyl]pyrrolo[1,2-d][1,2,4]triazin-4- |
| | yl}amino)pyrrolidin-2-one |
| 175 | (3S,4R)-3-({1-[2-(difluoromethoxy)-4-methoxyphenyl]pyrrolo[1,2- |
| | d][1,2,4]triazin-4-yl}amino)-1-ethylpiperidin-4-ol |
| 176 | [(1R,3S)-3-({1-[2-(difluoromethoxy)-4-methoxyphenyl]pyrrolo[1,2- |
| | d][1,2,4]triazin-4-yl}amino)cyclohexyl]methanol |
| 177 | (1R,2R)-2-({1-[2-(difluoromethoxy)-4-methoxyphenyl]pyrrolo[1,2- |
| | d][1,2,4]triazin-4-yl}amino)cyclohexan-1-ol |
| 178 | 1-[2-(cyclopropyloxy)-4-(trifluoromethyl)phenyl]-N-[(3R)-1-ethylpiperidin-3- |
| | yl]pyrrolo[1,2-d][1,2,4]triazin-4-amine |
| 179 | 2-[(3R)-3-({1-[2-(cyclopropyloxy)-4-(trifluoromethyl)phenyl]pyrrolo[1,2- |
| | d][1,2,4]triazin-4-yl}amino)piperidin-1-yl]ethan-1-ol |
| 180 | (1S,2S)-2-({1-[4-methoxy-2-(trifluoromethoxy)phenyl]pyrrolo[1,2- |
| | d][1,2,4]triazin-4-yl}amino)-1-methylcyclohexan-1-ol OR (1R,2R)-2-({1-[4- |
| | methoxy-2-(trifluoromethoxy)phenyl]pyrrolo[1,2-d][1,2,4]triazin-4-yl}amino)- |
| | 1-methylcyclohexan-1-ol |
| 181 | (1R,2R)-2-({1-[4-methoxy-2-(trifluoromethoxy)phenyl]pyrrolo[1,2- |
| | d][1,2,4]triazin-4-yl}amino)-1-methylcyclohexan-1-ol OR (1S,2S)-2-({1-[4- |
| | methoxy-2-(trifluoromethoxy)phenyl]pyrrolo[1,2-d][1,2,4]triazin-4-yl}amino)- |
| | 1-methylcyclohexan-1-ol |
| 182 | 1-[2-(cyclopropyloxy)-4-methoxyphenyl]-N-[(3R)-1-ethylpiperidin-3- |
| | yl]pyrrolo[1,2-d][1,2,4]triazin-4-amine |
| 183 | [(1s,3s)-3-({1-[2-(difluoromethoxy)-4-methylphenyl]imidazo[1,5- |
| | d][1,2,4]triazin-4-yl}amino)cyclobutyl]methanol |
| 184 | 2-[(3R)-3-({1-[2-(difluoromethoxy)-4-methylphenyl]imidazo[1,5- |
| | d][1,2,4]triazin-4-yl}amino)piperidin-1-yl]ethan-1-ol |
| 185 | 2-[(3R)-3-({1-[4-cyclopropyl-2-(difluoromethoxy)phenyl]imidazo[1,5- |
| | d][1,2,4]triazin-4-yl}amino)piperidin-1-yl]ethan-1-ol |
| 186 | 2-[(3R)-3-({1-[2-(difluoromethoxy)-4-(trifluoromethyl)phenyl]imidazo[1,5- |
| | d][1,2,4]triazin-4-yl}amino)piperidin-1-yl]ethan-1-ol |
| 187 | (1s,3s)-3-({1-[2-(difluoromethoxy)-4-(trifluoromethyl)phenyl]imidazo[1,5- |
| | d][1,2,4]triazin-4-yl}amino)-1-methylcyclobutan-1-ol |
| 188 | 1-[2-(difluoromethoxy)-4-(trifluoromethyl)phenyl]-N-[(3R)-oxan-3- |
| | yl]imidazo[1,5-d][1,2,4]triazin-4-amine |
| 189 | 1-[4-chloro-2-(difluoromethoxy)phenyl]-N-[(3R)-oxan-3-yl]imidazo[1,5- |
| | d][1,2,4]triazin-4-amine |
| 190 | 1-[4-chloro-2-(difluoromethoxy)phenyl]-N-[(3R)-1-methylpiperidin-3- |
| | yl]imidazo[1,5-d][1,2,4]triazin-4-amine |
| 191 | 1-[4-chloro-2-(difluoromethoxy)phenyl]-N-[(3R)-1-ethylpiperidin-3- |
| | yl]imidazo[1,5-d][1,2,4]triazin-4-amine |
| 192 | 2-[(3R)-3-({1-[4-chloro-2-(difluoromethoxy)phenyl]imidazo[1,5- |
| | d][1,2,4]triazin-4-yl}amino)piperidin-1-yl]ethan-1-ol |
| 193 | 1-[4-chloro-2-(difluoromethoxy)phenyl]-N-[(3R,5R)-5-fluoro-1- |
| | methylpiperidin-3-yl]imidazo[1,5-d][1,2,4]triazin-4-amine |
| 194 | (1s,3s)-3-({1-[4-chloro-2-(difluoromethoxy)phenyl]imidazo[1,5- |
| | d][1,2,4]triazin-4-yl}amino)-1-methylcyclobutan-1-ol |
| 195 | 1-[4-bromo-2-(difluoromethoxy)phenyl]-N-[(3R)-1-methylpiperidin-3- |
| | yl]imidazo[1,5-d][1,2,4]triazin-4-amine |
| 196 | 1-[4-bromo-2-(difluoromethoxy)phenyl]-N-[(3R,5R)-1-ethyl-5- |
| | fluoropiperidin-3-yl]imidazo[1,5-d][1,2,4]triazin-4-amine |
| 197 | N-[(3R)-1-methylpiperidin-3-yl]-1-[2-(trifluoromethoxy)-4- |
| | (trifluoromethyl)phenyl]imidazo[1,5-d][1,2,4]triazin-4-amine |
| 198 | 2-[(3R)-3-({1-[4-bromo-2-(difluoromethoxy)phenyl]imidazo[1,5- |
| | d][1,2,4]triazin-4-yl}amino)piperidin-1-yl]ethan-1-ol |
| 199 | (1s,3s)-3-({1-[4-bromo-2-(difluoromethoxy)phenyl]imidazo[1,5- |
| | d][1,2,4]triazin-4-yl}amino)-1-methylcyclobutan-1-ol |
| 200 | 1-[4-bromo-2-(trifluoromethyl)phenyl]-N-[(3R)-1-methylpiperidin-3- |
| | yl]imidazo[1,5-d][1,2,4]triazin-4-amine |
| 201 | 2-[(3R)-3-({1-[4-bromo-2-(trifluoromethyl)phenyl]imidazo[1,5- |
| | d][1,2,4]triazin-4-yl}amino)piperidin-1-yl]ethan-1-ol |
| 202 | 1-[2-(difluoromethoxy)-4-methylphenyl]-N-[(3R)-1-methylpiperidin-3- |
| | yl]imidazo[1,5-d][1,2,4]triazin-4-amine |
| 203 | 1-[4-cyclopropyl-2-(difluoromethoxy)phenyl]-N-[(3R)-1-methylpiperidin-3- |
| | yl]imidazo[1,5-d][1,2,4]triazin-4-amine |
| 204 | (1s,3s)-3-({1-[4-bromo-2-(trifluoromethyl)phenyl]imidazo[1,5- |
| | d][1,2,4]triazin-4-yl}amino)-1-methylcyclobutan-1-ol |
| 205 | 1-[4-bromo-2-(trifluoromethyl)phenyl]-N-[(3R,5R)-1-ethyl-5-fluoropiperidin- |
| | 3-yl]imidazo[1,5-d][1,2,4]triazin-4-amine |
| 206 | N-[(3R)-1-methylpiperidin-3-yl]-1-[4-methyl-2- |
| | (trifluoromethyl)phenyl]imidazo[1,5-d][1,2,4]triazin-4-amine |
| 207 | 1-[4-cyclopropyl-2-(trifluoromethyl)phenyl]-N-[(3R)-1-methylpiperidin-3- |
| | yl]imidazo[1,5-d][1,2,4]triazin-4-amine |
| 208 | 1-[4-methoxy-2-(trifluoromethoxy)phenyl]-N-[(3R)-1-methylpiperidin-3- |
| | yl]imidazo[1,5-d][1,2,4]triazin-4-amine |
| 209 | N-[(3R)-1-ethylpiperidin-3-yl]-1-[4-methoxy-2- |
| | (trifluoromethoxy)phenyl]imidazo[1,5-d][1,2,4]triazin-4-amine |
| 210 | N-[(3R,5R)-5-fluoro-1-methylpiperidin-3-yl]-1-[4-methoxy-2- |
| | (trifluoromethoxy)phenyl]imidazo[1,5-d][1,2,4]triazin-4-amine |
| 211 | 2-[(3R)-3-({1-[4-methoxy-2-(trifluoromethoxy)phenyl]imidazo[1,5- |
| | d][1,2,4]triazin-4-yl}amino)piperidin-1-yl]ethan-1-ol |
| 212 | N-[(3R)-1-ethylpiperidin-3-yl]-1-[4-methoxy-2- |
| | (trifluoromethyl)phenyl]imidazo[1,5-d][1,2,4]triazin-4-amine |
| 213 | 2-[(3R)-3-({1-[4-methoxy-2-(trifluoromethyl)phenyl]imidazo[1,5- |
| | d][1,2,4]triazin-4-yl}amino)piperidin-1-yl]ethan-1-ol |
| 214 | (1s,3s)-3-({1-[4-methoxy-2-(trifluoromethyl)phenyl]imidazo[1,5- |
| | d][1,2,4]triazin-4-yl}amino)-1-methylcyclobutan-1-ol |
| 215 | (1S,3R)-3-({1-[2,4-bis(trifluoromethyl)phenyl]imidazo[1,5-d][1,2,4]triazin-4- |
| | yl}amino)cyclohexan-1-ol |
| 216 | 1-[2-(difluoromethoxy)-4-methoxyphenyl]-N-[(3R)-1-methylpiperidin-3- |
| | yl]imidazo[1,5-d][1,2,4]triazin-4-amine |
| 217 | 1-[2,4-bis(trifluoromethyl)phenyl]-N-[(3R)-1-methylpiperidin-3- |
| | yl]imidazo[1,5-d][1,2,4]triazin-4-amine |
| 218 | (1s,3s)-3-({1-[2,4-bis(trifluoromethyl)phenyl]imidazo[1,5-d][1,2,4]triazin-4- |
| | yl}amino)-1-methylcyclobutan-1-ol |
| 219 | (1s,3s)-3-({1-[4-methoxy-2-(trifluoromethoxy)phenyl]imidazo[1,5- |
| | d][1,2,4]triazin-4-yl}amino)-1-methylcyclobutan-1-ol |
| 220 | 1-[4-methoxy-2-(trifluoromethoxy)phenyl]-N-(1H-pyrazol-3-yl)imidazo[1,5- |
| | d][1,2,4]triazin-4-amine |
| 221 | (1R,2R)-2-({1-[4-methoxy-2-(trifluoromethoxy)phenyl]imidazo[1,5- |
| | d][1,2,4]triazin-4-yl}amino)cyclopentan-1-ol |
| 222 | (1S,3R)-3-({1-[4-methoxy-2-(trifluoromethoxy)phenyl]imidazo[1,5- |
| | d][1,2,4]triazin-4-yl}amino)cyclohexan-1-ol |
| 223 | (1R,2R)-2-({1-[4-methoxy-2-(trifluoromethoxy)phenyl]imidazo[1,5- |
| | d][1,2,4]triazin-4-yl}amino)cyclohexan-1-ol |
| 224 | 1-[2-(difluoromethoxy)-4-methoxyphenyl]-N-[(3R,5R)-5-fluoro-1- |
| | methylpiperidin-3-yl]imidazo[1,5-d][1,2,4]triazin-4-amine |
| 225 | 2-[(3R)-3-({1-[2-(difluoromethoxy)-4-methoxyphenyl]imidazo[1,5- |
| | d][1,2,4]triazin-4-yl}amino)piperidin-1-yl]ethan-1-ol |
| 226 | (1s,3s)-3-({1-[2-(difluoromethoxy)-4-methoxyphenyl]imidazo[1,5- |
| | d][1,2,4]triazin-4-yl}amino)-1-methylcyclobutan-1-ol |
| 227 | 1-[2-(difluoromethoxy)-4-methoxyphenyl]-N-(1H-pyrazol-3-yl)imidazo[1,5- |
| | d][1,2,4]triazin-4-amine |
| 228 | 1-[4-methoxy-2-(trifluoromethyl)phenyl]-N-(1H-pyrazol-3-yl)imidazo[1,5- |
| | d][1,2,4]triazin-4-amine |
| 229 | N-[(3R)-1-ethylpiperidin-3-yl]-1-[2-(trifluoromethoxy)-4- |
| | (trifluoromethyl)phenyl]imidazo[1,5-d][1,2,4]triazin-4-amine |
| 230 | N-[1-(3-methoxyphenyl)ethyl]-1-[4-methoxy-2- |
| | (trifluoromethoxy)phenyl]imidazo[1,5-d][1,2,4]triazin-4-amine |
| 231 | 1-[4-methoxy-2-(trifluoromethoxy)phenyl]-N-(1-phenylethyl)imidazo[1,5- |
| | d][1,2,4]triazin-4-amine |
| 232 | 1-[4-methoxy-2-(trifluoromethoxy)phenyl]-N-[1-(pyridin-3- |
| | yl)ethyl]imidazo[1,5-d][1,2,4]triazin-4-amine |
| 233 | 3-[({1-[4-methoxy-2-(trifluoromethoxy)phenyl]imidazo[1,5-d][1,2,4]triazin-4- |
| | yl}amino)methyl]phenol |
| 234 | 1-[4-methoxy-2-(trifluoromethoxy)phenyl]-N-[(1H-pyrazol-3- |
| | yl)methyl]imidazo[1,5-d][1,2,4]triazin-4-amine |
| 235 | 3-({1-[4-methoxy-2-(trifluoromethyl)phenyl]imidazo[1,5-d][1,2,4]triazin-4- |
| | yl}amino)pyrrolidin-2-one |
| 236 | (1s,3s)-3-({1-[4-methoxy-2-(2,2,2-trifluoroethyl)phenyl]imidazo[1,5- |
| | d][1,2,4]triazin-4-yl}amino)-1-methylcyclobutan-1-ol |
| 237 | (1R,2R)-2-({1-[4-methoxy-2-(2,2,2-trifluoroethyl)phenyl]imidazo[1,5- |
| | d][1,2,4]triazin-4-yl}amino)cyclohexan-1-ol |
| 238 | 3-({1-[4-methoxy-2-(2,2,2-trifluoroethyl)phenyl]imidazo[1,5-d][1,2,4]triazin- |
| | 4-yl}amino)pyrrolidin-2-one |
| 239 | 2-[(3R)-3-({1-[2-(trifluoromethoxy)-4-(trifluoromethyl)phenyl]imidazo[1,5- |
| | d][1,2,4]triazin-4-yl}amino)piperidin-1-yl]ethan-1-ol |
| 240 | (1S,3R)-3-({1-[2-(trifluoromethoxy)-4-(trifluoromethyl)phenyl]imidazo[1,5- |
| | d][1,2,4]triazin-4-yl}amino)cyclohexan-1-ol |
| 241 | (1s,3s)-1-methyl-3-({1-[2-(trifluoromethoxy)-4- |
| | (trifluoromethyl)phenyl]imidazo[1,5-d][1,2,4]triazin-4-yl}amino)cyclobutan- |
| | 1-ol |
| 242 | (1R,2R)-2-({1-[2-(trifluoromethoxy)-4-(trifluoromethyl)phenyl]imidazo[1,5- |
| | d][1,2,4]triazin-4-yl}amino)cyclohexan-1-ol |
| 243 | (1R,2R)-2-({1-[2,4-bis(trifluoromethyl)phenyl]imidazo[1,5-d][1,2,4]triazin-4- |
| | yl}amino)cyclohexan-1-ol |
| 244 | (1s,3s)-3-({1-[4-(difluoromethoxy)-2-(trifluoromethoxy)phenyl]imidazo[1,5- |
| | d][1,2,4]triazin-4-yl}amino)-1-methylcyclobutan-1-ol |
| 245 | (1S,3R)-3-({1-[4-(difluoromethoxy)-2-(trifluoromethoxy)phenyl]imidazo[1,5- |
| | d][1,2,4]triazin-4-yl}amino)cyclohexan-1-ol |
| 246 | (1R,2R)-2-({1-[4-(difluoromethoxy)-2-(trifluoromethoxy)phenyl]imidazo[1,5- |
| | d][1,2,4]triazin-4-yl}amino)cyclohexan-1-ol |
| 247 | 3-[(1S)-1-({1-[4-methoxy-2-(trifluoromethoxy)phenyl]imidazo[1,5- |
| | d][1,2,4]triazin-4-yl}amino)ethyl]phenol OR 3-[(1R)-1-({1-[4-methoxy-2- |
| | (trifluoromethoxy)phenyl]imidazo[1,5-d][1,2,4]triazin-4- |
| | yl}amino)ethyl]phenol |
| 248 | 3-[(1R)-1-({1-[4-methoxy-2-(trifluoromethoxy)phenyl]imidazo[1,5- |
| | d][1,2,4]triazin-4-yl}amino)ethyl]phenol OR 3-[(1S)-1-({1-[4-methoxy-2- |
| | (trifluoromethoxy)phenyl]imidazo[1,5-d][1,2,4]triazin-4- |
| | yl}amino)ethyl]phenol |
| 249 | (3S,4R)-4-({1-[4-methoxy-2-(2,2,2-trifluoroethyl)phenyl]imidazo[1,5- |
| | d][1,2,4]triazin-4-yl}amino)oxan-3-ol |
| 250 | 1-[4-methoxy-2-(2,2,2-trifluoroethyl)phenyl]-N-[(3R)-1-methylpiperidin-3- |
| | yl]imidazo[1,5-d][1,2,4]triazin-4-amine |
| 251 | 3-[1-({1-[4-methoxy-2-(trifluoromethyl)phenyl]imidazo[1,5-d][1,2,4]triazin-4- |
| | yl}amino)ethyl]phenol |
| 252 | 1-[2,4-bis(trifluoromethyl)phenyl]-N-[(3R,5R)-5-fluoro-1-methylpiperidin-3- |
| | yl]imidazo[1,5-d][1,2,4]triazin-4-amine |
| 253 | 2-[(3R)-3-({1-[2,4-bis(trifluoromethyl)phenyl]imidazo[1,5-d][1,2,4]triazin-4- |
| | yl}amino)piperidin-1-yl]ethan-1-ol |
| 254 | 1-[2,4-bis(trifluoromethyl)phenyl]-N-[(3R)-oxan-3-yl]imidazo[1,5- |
| | d][1,2,4]triazin-4-amine |
| 255 | [(1s,3s)-3-({1-[2-(trifluoromethoxy)-4-(trifluoromethyl)phenyl]imidazo[1,5- |
| | d][1,2,4]triazin-4-yl}amino)cyclobutyl]methanol |
| 256 | 3-({1-[2-(trifluoromethoxy)-4-(trifluoromethyl)phenyl]imidazo[1,5- |
| | d][1,2,4]triazin-4-yl}amino)pyrrolidin-2-one |
| 257 | [(1s,3s)-3-({1-[2,4-bis(trifluoromethyl)phenyl]imidazo[1,5-d][1,2,4]triazin-4- |
| | yl}amino)cyclobutyl]methanol |
| 258 | 2-[(3R)-3-({1-[4-(difluoromethoxy)-2-(trifluoromethoxy)phenyl]imidazo[1,5- |
| | d][1,2,4]triazin-4-yl}amino)piperidin-1-yl]ethan-1-ol |
| 259 | 3-[1-({1-[4-(difluoromethoxy)-2-(trifluoromethoxy)phenyl]imidazo[1,5- |
| | d][1,2,4]triazin-4-yl}amino)ethyl]phenol |
| 260 | 3-({1-[4-(difluoromethoxy)-2-(trifluoromethoxy)phenyl]imidazo[1,5- |
| | d][1,2,4]triazin-4-yl}amino)pyrrolidin-2-one |
| 261 | N-[(3R)-1-ethylpiperidin-3-yl]-1-[2-(2,2,2-trifluoroethyl)-4- |
| | (trifluoromethyl)phenyl]imidazo[1,5-d][1,2,4]triazin-4-amine |
| 262 | (1S,3R)-3-({1-[2-(2,2,2-trifluoroethyl)-4-(trifluoromethyl)phenyl]imidazo[1,5- |
| | d][1,2,4]triazin-4-yl}amino)cyclohexan-1-ol |
| 263 | (1s,3s)-1-methyl-3-({1-[2-(2,2,2-trifluoroethyl)-4- |
| | (trifluoromethyl)phenyl]imidazo[1,5-d][1,2,4]triazin-4-yl}amino)cyclobutan- |
| | 1-ol |
| 264 | [(1s,3s)-3-({1-[2-(2,2,2-trifluoroethyl)-4-(trifluoromethyl)phenyl]imidazo[1,5- |
| | d][1,2,4]triazin-4-yl}amino)cyclobutyl]methanol |
| 265 | 3-({1-[2-(2,2,2-trifluoroethyl)-4-(trifluoromethyl)phenyl]imidazo[1,5- |
| | d][1,2,4]triazin-4-yl}amino)pyrrolidin-2-one |
| 266 | 3-[1-({1-[2-(2,2,2-trifluoroethyl)-4-(trifluoromethyl)phenyl]imidazo[1,5- |
| | d][1,2,4]triazin-4-yl}amino)ethyl]phenol |
| 267 | N-(1H-pyrazol-3-yl)-1-[2-(2,2,2-trifluoroethyl)-4- |
| | (trifluoromethyl)phenyl]imidazo[1,5-d][1,2,4]triazin-4-amine |
| 268 | (3S,4R)-4-({1-[2-(difluoromethoxy)-4-methoxyphenyl]imidazo[1,5- |
| | d][1,2,4]triazin-4-yl}amino)oxan-3-ol |
| 269 | 3-({1-[2-(difluoromethoxy)-4-methoxyphenyl]imidazo[1,5-d][1,2,4]triazin-4- |
| | yl}amino)pyrrolidin-2-one |
| 270 | (1R,2R)-2-({1-[2-(difluoromethoxy)-4-methoxyphenyl]imidazo[1,5- |
| | d][1,2,4]triazin-4-yl}amino)cyclohexan-1-ol |
| 271 | 1-[2,4-bis(trifluoromethyl)phenyl]-N-[(3R)-1-ethylpiperidin-3-yl]imidazo[1,5- |
| | d][1,2,4]triazin-4-amine |
| 272 | 3-({1-[2,4-bis(trifluoromethyl)phenyl]imidazo[1,5-d][1,2,4]triazin-4- |
| | yl}amino)-1-methylpiperidin-2-one |
| 273 | (3S,4R)-4-({1-[2,4-bis(trifluoromethyl)phenyl]imidazo[1,5-d][1,2,4]triazin-4- |
| | yl}amino)oxan-3-ol |
| 274 | (3S,4S)-3-({1-[2,4-bis(trifluoromethyl)phenyl]imidazo[1,5-d][1,2,4]triazin-4- |
| | yl}amino)oxan-4-ol |
| 275 | (1s,4s)-4-({1-[2,4-bis(trifluoromethyl)phenyl]imidazo[1,5-d][1,2,4]triazin-4- |
| | yl}amino)cyclohexan-1-ol |
| 276 | 3-({1-[2,4-bis(trifluoromethyl)phenyl]imidazo[1,5-d][1,2,4]triazin-4- |
| | yl}amino)piperidin-2-one |
| 277 | N-[(3R,5R)-5-fluoro-1-methylpiperidin-3-yl]-1-[4-methoxy-2-(2,2,2- |
| | trifluoroethyl)phenyl]imidazo[1,5-d][1,2,4]triazin-4-amine |
| 278 | 2-[(3R)-3-({1-[4-methoxy-2-(2,2,2-trifluoroethyl)phenyl]imidazo[1,5- |
| | d][1,2,4]triazin-4-yl}amino)piperidin-1-yl]ethan-1-ol |
| 279 | 8-methyl-N-[(3R)-1-methylpiperidin-3-yl]-1-[2-(2,2,2-trifluoroethyl)-4- |
| | (trifluoromethyl)phenyl]imidazo[1,5-d][1,2,4]triazin-4-amine |
| 280 | (1R,2R)-2-({8-methyl-1-[2-(2,2,2-trifluoroethyl)-4- |
| | (trifluoromethyl)phenyl]imidazo[1,5-d][1,2,4]triazin-4-yl}amino)cyclohexan- |
| | 1-ol |
| 281 | 1-[4-methoxy-2-(pentafluoroethyl)phenyl]-N-[(3R)-1-methylpiperidin-3- |
| | yl]imidazo[1,5-d][1,2,4]triazin-4-amine |
| 282 | (1R,2R)-2-({1-[2-(2,2,2-trifluoroethyl)-4-(trifluoromethyl)phenyl]imidazo[1,5- |
| | d][1,2,4]triazin-4-yl}amino)cyclohexan-1-ol |
| 283 | (1S,2S)-1-methyl-2-({1-[2-(2,2,2-trifluoroethyl)-4- |
| | (trifluoromethyl)phenyl]imidazo[1,5-d][1,2,4]triazin-4-yl}amino)cyclohexan- |
| | 1-ol AND (1R,2R)-1-methyl-2-({1-[2-(2,2,2-trifluoroethyl)-4- |
| | (trifluoromethyl)phenyl]imidazo[1,5-d][1,2,4]triazin-4-yl}amino)cyclohexan- |
| | 1-ol |
| 284 | 3-({1-[4-methoxy-2-(trifluoromethoxy)phenyl]imidazo[1,5-d][1,2,4]triazin-4- |
| | yl}amino)pyrrolidin-2-one |
| 285 | 1-[2,4-bis(trifluoromethyl)phenyl]-N-(1H-pyrazol-3-yl)imidazo[1,5- |
| | d][1,2,4]triazin-4-amine |
| 286 | (1R,2R)-2-({1-[4-methyl-2-(2,2,2-trifluoroethyl)phenyl]imidazo[1,5- |
| | d][1,2,4]triazin-4-yl}amino)cyclohexan-1-ol |
| 287 | 3-({1-[4-chloro-2-(trifluoromethoxy)phenyl]imidazo[1,5-d][1,2,4]triazin-4- |
| | yl}amino)pyrrolidin-2-one |
| 288 | 3-({1-[4-methyl-2-(2,2,2-trifluoroethyl)phenyl]imidazo[1,5-d][1,2,4]triazin-4- |
| | yl}amino)pyrrolidin-2-one |
| 289 | (3S,4R)-1-methyl-3-({1-[2-(2,2,2-trifluoroethyl)-4- |
| | (trifluoromethyl)phenyl]imidazo[1,5-d][1,2,4]triazin-4-yl}amino)piperidin-4- |
| | ol |
| 290 | (1S,2S)-2-({1-[2-(trifluoromethoxy)-4-(trifluoromethyl)phenyl]imidazo[1,5- |
| | d][1,2,4]triazin-4-yl}amino)cyclohexan-1-ol |
| 291 | [(1s,3s)-3-({1-[4-methoxy-2-(trifluoromethoxy)phenyl]imidazo[1,5- |
| | d][1,2,4]triazin-4-yl}amino)cyclobutyl]methanol |
| 292 | (3S,4R)-4-({1-[4-methoxy-2-(trifluoromethoxy)phenyl]imidazo[1,5- |
| | d][1,2,4]triazin-4-yl}amino)oxan-3-ol |
| 293 | (1S,2S)-2-({1-[2,4-bis(trifluoromethyl)phenyl]imidazo[1,5-d][1,2,4]triazin-4- |
| | yl}amino)cyclohexan-1-ol |
| 294 | 3-({1-[2,4-bis(trifluoromethyl)phenyl]imidazo[1,5-d][1,2,4]triazin-4- |
| | yl}amino)pyrrolidin-2-one |
| 295 | 1-[4-(difluoromethoxy)-2-(trifluoromethoxy)phenyl]-N-[(3R)-1- |
| | methylpiperidin-3-yl]imidazo[1,5-d][1,2,4]triazin-4-amine |
| 296 | 1-[4-(difluoromethoxy)-2-(trifluoromethoxy)phenyl]-N-(1H-pyrazol-3- |
| | yl)imidazo[1,5-d][1,2,4]triazin-4-amine |
| 297 | 3-[1-({1-[4-methyl-2-(2,2,2-trifluoroethyl)phenyl]imidazo[1,5-d][1,2,4]triazin- |
| | 4-yl}amino)ethyl]phenol |
| 298 | (1s,3s)-3-({1-[4-chloro-2-(trifluoromethyl)phenyl]imidazo[1,5-d][1,2,4]triazin- |
| | 4-yl}amino)-1-methylcyclobutan-1-ol |
| 299 | (1s,3s)-3-({1-[4-chloro-2-(trifluoromethoxy)phenyl]imidazo[1,5- |
| | d][1,2,4]triazin-4-yl}amino)-1-methylcyclobutan-1-ol |
| 300 | (1R,2R)-2-({1-[4-chloro-2-(trifluoromethoxy)phenyl]imidazo[1,5- |
| | d][1,2,4]triazin-4-yl}amino)cyclohexan-1-ol |
| 301 | 2-[(3R)-3-({1-[2-(2,2,2-trifluoroethyl)-4-(trifluoromethyl)phenyl]imidazo[1,5- |
| | d][1,2,4]triazin-4-yl}amino)piperidin-1-yl]ethan-1-ol |
| 302 | (1S,3R)-3-({1-[4-methoxy-2-(2,2,2-trifluoroethyl)phenyl]imidazo[1,5- |
| | d][1,2,4]triazin-4-yl}amino)cyclohexan-1-ol |
| 303 | 3-[1-({1-[4-methoxy-2-(2,2,2-trifluoroethyl)phenyl]imidazo[1,5- |
| | d][1,2,4]triazin-4-yl}amino)ethyl]phenol |
| 304 | 1-[4-methoxy-2-(2,2,2-trifluoroethoxy)phenyl]-N-[(3R)-1-methylpiperidin-3- |
| | yl]imidazo[1,5-d][1,2,4]triazin-4-amine |
| 305 | (1s,3s)-3-({1-[4-methoxy-2-(2,2,2-trifluoroethoxy)phenyl]imidazo[1,5- |
| | d][1,2,4]triazin-4-yl}amino)-1-methylcyclobutan-1-of |
| 306 | (1R,2R)-2-({1-[4-bromo-2-(difluoromethoxy)phenyl]imidazo[1,5- |
| | d][1,2,4]triazin-4-yl}amino)cyclohexan-1-ol |
| 307 | (1R,2R)-2-({1-[4-bromo-2-(trifluoromethoxy)phenyl]imidazo[1,5- |
| | d][1,2,4]triazin-4-yl}amino)cyclohexan-1-ol |
| 308 | 1-[4-methoxy-2-(trifluoromethoxy)phenyl]-N-[(1S)-1-(pyridin-3- |
| | yl)ethyl]imidazo[1,5-d][1,2,4]triazin-4-amine OR 1-[4-methoxy-2- |
| | (trifluoromethoxy)phenyl]-N-[(1R)-1-(pyridin-3-yl)ethyl]imidazo[1,5- |
| | d][1,2,4]triazin-4-amine |
| 309 | 1-[4-methoxy-2-(trifluoromethoxy)phenyl]-N-[(1R)-1-(pyridin-3- |
| | yl)ethyl]imidazo[1,5-d][1,2,4]triazin-4-amine OR 1-[4-methoxy-2- |
| | (trifluoromethoxy)phenyl]-N-[(1S)-1-(pyridin-3-yl)ethyl]imidazo[1,5- |
| | d][1,2,4]triazin-4-amine |
| 310 | (1S,2S)-2-({1-[4-methoxy-2-(trifluoromethoxy)phenyl]imidazo[1,5- |
| | d][1,2,4]triazin-4-yl}amino)-1-methylcyclohexan-1-ol OR (1R,2R)-2-({1-[4- |
| | methoxy-2-(trifluoromethoxy)phenyl]imidazo[1,5-d][1,2,4]triazin-4- |
| | yl}amino)-1-methylcyclohexan-1-ol |
| 311 | (1R,2R)-2-({1-[4-methoxy-2-(trifluoromethoxy)phenyl]imidazo[1,5- |
| | d][1,2,4]triazin-4-yl}amino)-1-methylcyclohexan-1-ol OR (1S,2S)-2-({1-[4- |
| | methoxy-2-(trifluoromethoxy)phenyl]imidazo[1,5-d][1,2,4]triazin-4- |
| | yl}amino)-1-methylcyclohexan-1-ol |
| 312 | 1-[4-bromo-2-(trifluoromethoxy)phenyl]-N-[(3R)-1-methylpiperidin-3- |
| | yl]imidazo[1,5-d][1,2,4]triazin-4-amine |
| 313 | (3S,4R)-4-({1-[2-(2,2,2-trifluoroethyl)-4-(trifluoromethyl)phenyl]imidazo[1,5- |
| | d][1,2,4]triazin-4-yl}amino)oxan-3-ol |
| 314 | [(1s,3s)-3-({1-[4-methoxy-2-(2,2,2-trifluoroethyl)phenyl]imidazo[1,5- |
| | d][1,2,4]triazin-4-yl}amino)cyclobutyl]methanol |
| 315 | (1s,3s)-3-({1-[4-methoxy-2-(pentafluoroethyl)phenyl]imidazo[1,5- |
| | d][1,2,4]triazin-4-yl}amino)-1-methylcyclobutan-1-ol |
| 316 | (1R,2R)-2-({1-[4-methoxy-2-(pentafluoroethyl)phenyl]imidazo[1,5- |
| | d][1,2,4]triazin-4-yl}amino)cyclohexan-1-ol |
| 317 | N-[(3R)-1-methylpiperidin-3-yl]-1-[4-methyl-2-(2,2,2- |
| | trifluoroethyl)phenyl]imidazo[1,5-d][1,2,4]triazin-4-amine |
| 318 | (1s,3s)-1-methyl-3-({1-[4-methyl-2-(2,2,2-trifluoroethyl)phenyl]imidazo[1,5- |
| | d][1,2,4]triazin-4-yl}amino)cyclobutan-1-ol |
| 319 | (1s,3s)-3-{[1-(2-cyclopropyl-4-methoxyphenyl)imidazo[1,5-d][1,2,4]triazin-4- |
| | yl]amino}-1-methylcyclobutan-1-ol |
| 320 | 3-{[1-(2-cyclopropyl-4-methoxyphenyl)imidazo[1,5-d][1,2,4]triazin-4- |
| | yl]amino}pyrrolidin-2-one |
| 321 | (1R,2R)-2-{[1-(2-cyclopropyl-4-methoxyphenyl)imidazo[1,5-d][1,2,4]triazin- |
| | 4-yl]amino}cyclohexan-1-ol |
| 322 | (3S,4R)-4-({1-[2-(trifluoromethoxy)-4-(trifluoromethyl)phenyl]imidazo[1,5- |
| | d][1,2,4]triazin-4-yl}amino)oxan-3-ol |
| 323 | 3-[1-({1-[2-(trifluoromethoxy)-4-(trifluoromethyl)phenyl]imidazo[1,5- |
| | d][1,2,4]triazin-4-yl}amino)ethyl]phenol |
| 324 | N-[3-(methoxymethyl)cyclobutyl]-1-[2-(trifluoromethoxy)-4- |
| | (trifluoromethyl)phenyl]imidazo[1,5-d][1,2,4]triazin-4-amine |
| 325 | 2-[3-({1-[2-(trifluoromethoxy)-4-(trifluoromethyl)phenyl]imidazo[1,5- |
| | d][1,2,4]triazin-4-yl}amino)cyclobutyl]propan-2-ol |
| 326 | N-[3-(methoxymethyl)cyclobutyl]-1-[4-methoxy-2-(2,2,2- |
| | trifluoroethyl)phenyl]imidazo[1,5-d][1,2,4]triazin-4-amine |
| 327 | 2-[3-({1-[4-methoxy-2-(2,2,2-trifluoroethyl)phenyl]imidazo[1,5- |
| | d][1,2,4]triazin-4-yl}amino)cyclobutyl]propan-2-ol |
| 328 | 3-({1-[4-(difluoromethoxy)-2-(trifluoromethoxy)phenyl]imidazo[1,5- |
| | d][1,2,4]triazin-4-yl}amino)-1-methylpiperidin-2-one |
| 329 | 1-methyl-3-({1-[2-(2,2,2-trifluoroethyl)-4- |
| | (trifluoromethyl)phenyl]imidazo[1,5-d][1,2,4]triazin-4-yl}amino)piperidin-2- |
| | one |
| 330 | 3-({1-[4-methoxy-2-(2,2,2-trifluoroethyl)phenyl]imidazo[1,5-d][1,2,4]triazin- |
| | 4-yl}amino)-1-methylpiperidin-2-one |
| 331 | (3S,4R)-4-({1-[4-(difluoromethoxy)-2-(trifluoromethoxy)phenyl]imidazo[1,5- |
| | d][1,2,4]triazin-4-yl}amino)oxan-3-ol |
| 332 | (1s,3s)-1-methyl-3-({1-[4-methyl-2-(trifluoromethoxy)phenyl]imidazo[1,5- |
| | d][1,2,4]triazin-4-yl}amino)cyclobutan-1-ol |
| 333 | 3-({1-[2-cyclopropyl-4-(trifluoromethyl)phenyl]imidazo[1,5-d][1,2,4]triazin- |
| | 4-yl}amino)pyrrolidin-2-one |
| 334 | (3S,4R)-4-({1-[2-cyclopropyl-4-(trifluoromethyl)phenyl]imidazo[1,5- |
| | d][1,2,4]triazin-4-yl}amino)oxan-3-ol |
| 335 | [1-methyl-3-({1-[2-(trifluoromethoxy)-4-(trifluoromethyl)phenyl]imidazo[1,5- |
| | d][1,2,4]triazin-4-yl}amino)cyclobutyl]methanol |
| 336 | [(1r,3r)-3-({1-[4-methoxy-2-(2,2,2-trifluoroethyl)phenyl]imidazo[1,5- |
| | d][1,2,4]triazin-4-yl}amino)-1-methylcyclobutyl]methanol |
| 337 | [3-({1-[4-methoxy-2-(2,2,2-trifluoroethyl)phenyl]imidazo[1,5-d][1,2,4]triazin- |
| | 4-yl}amino)-1-methylcyclobutyl]methanol |
| 338 | 2-[3-({1-[4-methoxy-2-(trifluoromethoxy)phenyl]imidazo[1,5-d][1,2,4]triazin- |
| | 4-yl}amino)cyclobutyl]propan-2-ol |
| 339 | 2-[3-({1-[2-(2,2,2-trifluoroethyl)-4-(trifluoromethyl)phenyl]imidazo[1,5- |
| | d][1,2,4]triazin-4-yl}amino)cyclobutyl]propan-2-ol |
| 340 | [(1s,3s)-1-methyl-3-({1-[2-(trifluoromethoxy)-4- |
| | (trifluoromethyl)phenyl]imidazo[1,5-d][1,2,4]triazin-4- |
| | yl}amino)cyclobutyl]methanol |
| 341 | [(1s,3s)-3-({1-[4-methoxy-2-(2,2,2-trifluoroethyl)phenyl]imidazo[1,5- |
| | d][1,2,4]triazin-4-yl}amino)-1-methylcyclobutyl]methanol |
| 342 | [(1s,3s)-3-({1-[4-methoxy-2-(trifluoromethoxy)phenyl]imidazo[1,5- |
| | d][1,2,4]triazin-4-yl}amino)-1-methylcyclobutyl]methanol |
| 343 | [(1s,3s)-1-methyl-3-({1-[2-(2,2,2-trifluoroethyl)-4- |
| | (trifluoromethyl)phenyl]imidazo[1,5-d][1,2,4]triazin-4- |
| | yl}amino)cyclobutyl]methanol |
| 344 | (1s,3s)-3-({1-[2-(difluoromethoxy)-4-methylphenyl]imidazo[1,5- |
| | d][1,2,4]triazin-4-yl}amino)-1-methylcyclobutan-1-ol |
| 345 | (1s,3s)-3-({1-[4-cyclopropyl-2-(difluoromethoxy)phenyl]imidazo[1,5- |
| | d][1,2,4]triazin-4-yl}amino)-1-methylcyclobutan-1-ol |
| 346 | (1s,3s)-3-({1-[4-cyclopropyl-2-(trifluoromethoxy)phenyl]imidazo[1,5- |
| | d][1,2,4]triazin-4-yl}amino)-1-methylcyclobutan-1-ol |
| 347 | [(1s,3s)-3-({1-[4-methyl-2-(trifluoromethoxy)phenyl]imidazo[1,5- |
| | d][1,2,4]triazin-4-yl}amino)cyclobutyl]methanol |
| 348 | (1s,3s)-3-({1-[4-cyclopropyl-2-(2,2,2-trifluoroethyl)phenyl]imidazo[1,5- |
| | d][1,2,4]triazin-4-yl}amino)-1-methylcyclobutan-1-ol |
| 349 | [(1s,3s)-3-({1-[4-cyclopropyl-2-(2,2,2-trifluoroethyl)phenyl]imidazo[1,5- |
| | d][1,2,4]triazin-4-yl}amino)cyclobutyl]methanol |
| 350 | 3-({1-[4-cyclopropyl-2-(2,2,2-trifluoroethyl)phenyl]imidazo[1,5- |
| | d][1,2,4]triazin-4-yl}amino)pyrrolidin-2-one |
| 351 | (3S,4R)-4-({1-[4-cyclopropyl-2-(2,2,2-trifluoroethyl)phenyl]imidazo[1,5- |
| | d][1,2,4]triazin-4-yl}amino)oxan-3-ol |
| 352 | 1-[4-cyclopropyl-2-(2,2,2-trifluoroethyl)phenyl]-N-[(3R)-1-methylpiperidin-3- |
| | yl]imidazo[1,5-d][1,2,4]triazin-4-amine |
| 353 | (1S,3R)-3-({1-[4-cyclopropyl-2-(2,2,2-trifluoroethyl)phenyl]imidazo[1,5- |
| | d][1,2,4]triazin-4-yl}amino)cyclohexan-1-ol |
| 354 | 3-[1-({1-[4-cyclopropyl-2-(2,2,2-trifluoroethyl)phenyl]imidazo[1,5- |
| | d][1,2,4]triazin-4-yl}amino)ethyl]phenol |
| 355 | 2-[(3R)-3-({1-[4-methyl-2-(trifluoromethoxy)phenyl]imidazo[1,5- |
| | d][1,2,4]triazin-4-yl}amino)piperidin-1-yl]ethan-1-ol |
| 356 | 4-[2-(difluoromethoxy)-4-(trifluoromethyl)phenyl]-N-[(3R,SR)-1-ethyl-5- |
| | fluoropiperidin-3-yl]-2-methylpyrazolo[1,5-d][1,2,4]triazin-7-amine |
| 357 | 4-[4-bromo-2-(trifluoromethoxy)phenyl]-N-[(3R)-1-ethylpiperidin-3-yl]-2- |
| | methylpyrazolo[1,5-d][1,2,4]triazin-7-amine |
| 358 | 4-[4-chloro-2-(difluoromethoxy)phenyl]-2-methyl-N-[(3R)-1-methylpiperidin- |
| | 3-yl]pyrazolo[1,5-d][1,2,4]triazin-7-amine |
| 359 | 4-[2-(difluoromethyl)-4-(trifluoromethyl)phenyl]-2-methyl-N-[(3R)-1- |
| | methylpiperidin-3-yl]pyrazolo[1,5-d][1,2,4]triazin-7-amine |
| 360 | 4-[4-cyclopropyl-2-(difluoromethoxy)phenyl]-2-methyl-N-[(3R)-1- |
| | methylpiperidin-3-yl]pyrazolo[1,5-d][1,2,4]triazin-7-amine |
| 361 | 4-[2-(difluoromethoxy)-4-methylphenyl]-2-methyl-N-[(3R)-1- |
| | methylpiperidin-3-yl]pyrazolo[1,5-d][1,2,4]triazin-7-amine |
| 362 | 4-[4-chloro-2-(difluoromethyl)phenyl]-2-methyl-N-[(3R)-1-methylpiperidin-3- |
| | yl]pyrazolo[1,5-d][1,2,4]triazin-7-amine |
| 363 | 4-[4-cyclopropyl-2-(difluoromethyl)phenyl]-2-methyl-N-[(3R)-1- |
| | methylpiperidin-3-yl]pyrazolo[1,5-d][1,2,4]triazin-7-amine |
| 364 | 4-[2-(difluoromethyl)-4-methylphenyl]-2-methyl-N-[(3R)-1-methylpiperidin- |
| | 3-yl]pyrazolo[1,5-d][1,2,4]triazin-7-amine |
| 365 | N-[(3R)-1-ethylpiperidin-3-yl]-2-methyl-4-[2-(trifluoromethoxy)-4- |
| | (trifluoromethyl)phenyl]pyrazolo[1,5-d][1,2,4]triazin-7-amine |
| 366 | N-[(3R)-1-ethylpiperidin-3-yl]-2-methyl-4-[4-(methylsulfanyl)-2- |
| | (trifluoromethoxy)phenyl]pyrazolo[1,5-d][1,2,4]triazin-7-amine |
| 367 | N-[(3R)-1-ethylpiperidin-3-yl]-2-methyl-4-[4-methyl-2- |
| | (methylsulfanyl)phenyl]pyrazolo[1,5-d][1,2,4]triazin-7-amine |
| 368 | N-[(3R)-1-ethylpiperidin-3-yl]-4-[2-(methanesulfonyl)-4-methylphenyl]-2- |
| | methylpyrazolo[1,5-d][1,2,4]triazin-7-amine |
| 369 | N-[(3R)-1-ethylpiperidin-3-yl]-4-[4-(methanesulfonyl)-2- |
| | (trifluoromethoxy)phenyl]-2-methylpyrazolo[1,5-d][1,2,4]triazin-7-amine |
| 370 | N-[(3R)-1-ethylpiperidin-3-yl]-2-methyl-4-(2,3,4- |
| | trimethoxyphenyl)pyrazolo[1,5-d][1,2,4]triazin-7-amine |
| 371 | N-[(3R)-1-ethylpiperidin-3-yl]-4-[4-methoxy-2-(trifluoromethoxy)phenyl]-2- |
| | methylpyrazolo[1,5-d][1,2,4]triazin-7-amine |
| 372 | 2-(7-{[(3R)-1-ethylpiperidin-3-yl]amino}-2-methylpyrazolo[1,5- |
| | d][1,2,4]triazin-4-yl)-5-(trifluoromethyl)benzamide |
| 373 | N-[(3R)-1-ethylpiperidin-3-yl]-4-[4-methoxy-2-(trifluoromethyl)phenyl]-2- |
| | methylpyrazolo[1,5-d][1,2,4]triazin-7-amine |
| 374 | 4-(2-chloro-4-methoxyphenyl)-N-[(3R)-1-ethylpiperidin-3-yl]-2- |
| | methylpyrazolo[1,5-d][1,2,4]triazin-7-amine |
| 375 | 4-[2-chloro-4-(trifluoromethyl)phenyl]-N-[(3R)-1-ethylpiperidin-3-yl]-2- |
| | methylpyrazolo[1,5-d][1,2,4]triazin-7-amine |
| 376 | 4-(2,4-dichlorophenyl)-N-[(3R)-1-ethylpiperidin-3-yl]-2-methylpyrazolo[1,5- |
| | d][1,2,4]triazin-7-amine |
| 377 | 4-(4-chlorophenyl)-N-[(3R)-1-ethylpiperidin-3-yl]-2-methylpyrazolo[1,5- |
| | d][1,2,4]triazin-7-amine |
| 378 | 3-(difluoromethoxy)-4-(7-{[(3R)-1-ethylpiperidin-3-yl]amino}-2- |
| | methylpyrazolo[1,5-d][1,2,4]triazin-4-yl)benzonitrile |
| 379 | 4-(2-cyclopropyl-4-methoxyphenyl)-N-[(3R)-1-ethylpiperidin-3-yl]-2- |
| | methylpyrazolo[1,5-d][1,2,4]triazin-7-amine |
| 380 | (1s,3s)-3-({4-[2-(difluoromethoxy)-4-(trifluoromethyl)phenyl]-2- |
| | methylpyrazolo[1,5-d][1,2,4]triazin-7-yl}amino)-1-methylcyclobutan-1-ol |
| 381 | (1R,2R)-2-({4-[2-(difluoromethoxy)-4-(trifluoromethyl)phenyl]-2- |
| | methylpyrazolo[1,5-d][1,2,4]triazin-7-yl}amino)cyclohexan-1-ol |
| 382 | (1R,2R)-2-({2-methyl-4-[2-(trifluoromethoxy)-4- |
| | (trifluoromethyl)phenyl]pyrazolo[1,5-d][1,2,4]triazin-7-yl}amino)cyclohexan- |
| | 1-ol |
| 383 | 3-[1-({4-[2-(difluoromethoxy)-4-(trifluoromethyl)phenyl]-2- |
| | methylpyrazolo[1,5-d][1,2,4]triazin-7-yl}amino)ethyl]phenol |
| 384 | (3S,4R)-4-({4-[4-methoxy-2-(2,2,2-trifluoroethyl)phenyl]-2- |
| | methylpyrazolo[1,5-d][1,2,4]triazin-7-yl}amino)oxan-3-ol |
| 385 | (1R,2R)-2-({4-[4-methoxy-2-(2,2,2-trifluoroethyl)phenyl]-2- |
| | methylpyrazolo[1,5-d][1,2,4]triazin-7-yl}amino)cyclohexan-1-ol |
| 386 | 3-({4-[4-methoxy-2-(2,2,2-trifluoroethyl)phenyl]-2-methylpyrazolo[1,5- |
| | d][1,2,4]triazin-7-yl}amino)pyrrolidin-2-one |
| 387 | (1s,3s)-3-({4-[4-methoxy-2-(2,2,2-trifluoroethyl)phenyl]-2- |
| | methylpyrazolo[1,5-d][1,2,4]triazin-7-yl}amino)-1-methylcyclobutan-1-ol |
| 388 | (1s,3s)-1-methyl-3-({2-methyl-4-[2-(2,2,2-trifluoroethyl)-4- |
| | (trifluoromethyl)phenyl]pyrazolo[1,5-d][1,2,4]triazin-7-yl}amino)cyclobutan- |
| | 1-ol |
| 389 | (1R,2R)-2-({2-methyl-4-[2-(2,2,2-trifluoroethyl)-4- |
| | (trifluoromethyl)phenyl]pyrazolo[1,5-d][1,2,4]triazin-7-yl}amino)cyclohexan- |
| | 1-ol |
| 390 | (1s,3s)-1-methyl-3-({2-methyl-4-[4-methyl-2-(2,2,2- |
| | trifluoroethyl)phenyl]pyrazolo[1,5-d][1,2,4]triazin-7-yl}amino)cyclobutan-1- |
| | ol |
| 391 | (1s,3s)-3-({4-[4-methoxy-2-(trifluoromethoxy)phenyl]-2-methylpyrazolo[1,5- |
| | d][1,2,4]triazin-7-yl}amino)-1-methylcyclobutan-1-ol |
| 392 | (1R,2R)-2-({4-[4-methoxy-2-(trifluoromethoxy)phenyl]-2- |
| | methylpyrazolo[1,5-d][1,2,4]triazin-7-yl}amino)cyclohexan-1-ol |
| 393 | 3-({4-[2-(trifluoromethoxy)-4-(trifluoromethyl)phenyl]pyrazolo[1,5- |
| | d][1,2,4]triazin-7-yl}amino)pyrrolidin-2-one |
| 394 | (1R,2R)-2-({4-[2,4-bis(trifluoromethyl)phenyl]pyrazolo[1,5-d][1,2,4]triazin-7- |
| | yl}amino)cyclohexan-1-ol |
| 395 | 3-[1-({4-[2,4-bis(trifluoromethyl)phenyl]pyrazolo[1,5-d][1,2,4]triazin-7- |
| | yl}amino)ethyl]phenol |
| 396 | 3-({4-[2,4-bis(trifluoromethyl)phenyl]pyrazolo[1,5-d][1,2,4]triazin-7- |
| | yl}amino)pyrrolidin-2-one |
| 397 | (1s,3s)-1-methyl-3-({4-[2-(trifluoromethoxy)-4- |
| | (trifluoromethyl)phenyl]pyrazolo[1,5-d][1,2,4]triazin-7-yl}amino)cyclobutan- |
| | 1-ol |
| 398 | (1R,2R)-2-({4-[2-(trifluoromethoxy)-4-(trifluoromethyl)phenyl]pyrazolo[1,5- |
| | d][1,2,4]triazin-7-yl}amino)cyclohexan-1-ol |
| 399 | (1s,3s)-3-({1-[2-cyclopropyl-4-(trifluoromethyl)phenyl]imidazo[1,5- |
| | d][1,2,4]triazin-4-yl}amino)-1-methylcyclobutan-1-ol |
| 400 | (3S,4S)-3-({1-[2-(2,2,2-trifluoroethyl)-4-(trifluoromethyl)phenyl]imidazo[1,5- |
| | d][1,2,4]triazin-4-yl}amino)oxan-4-ol |
| 401 | (1S,2R)-2-({1-[2-(2,2,2-trifluoroethyl)-4-(trifluoromethyl)phenyl]imidazo[1,5- |
| | d][1,2,4]triazin-4-yl}amino)cyclohexan-1-ol |
| 402 | (3S,4R)-4-({1-[4-methyl-2-(2,2,2-trifluoroethyl)phenyl]imidazo[1,5- |
| | d][1,2,4]triazin-4-yl}amino)oxan-3-ol |

wherein a form of the compound is selected from the group consisting of a pharmaceutically acceptable salt, hydrate, solvate, racemate, enantiomer, diastereomer, stereoisomer, tautomer, and isotope enriched form thereof.

3. A compound selected from:

| Compound# | Name |
|---|---|
| 2 | N-[(3R)-1-methylpiperidin-3-yl]-1-[2-(2,2,2-trifluoroethyl)-4- |
| | (trifluoromethyl)phenyl]imidazo[1,5-d][1,2,4]triazin-4-amine |
| | formate |
| 12 | 1-[2-(difluoromethoxy)-4-methoxyphenyl]-7-methyl-N-[(3R)-1- |
| | methylpiperidin-3-yl]pyrrolo[1,2-d][1,2,4]triazin-4-amine formate |
| 13 | 1-[4-chloro-2-(difluoromethoxy)phenyl]-7-methyl-N-[(3R)-1- |
| | methylpiperidin-3-yl]pyrrolo[1,2-d][1,2,4]triazin-4-amine formate |
| 14 | 1-[4-cyclopropyl-2-(difluoromethyl)phenyl]-7-methyl-N-[(3R)-1- |
| | methylpiperidin-3-yl]pyrrolo[1,2-d][1,2,4]triazin-4-amine formate |
| 19 | 1-[2-fluoro-4-(trifluoromethyl)phenyl]-N-[(3R)-1-methylpiperidin-3- |
| | yl]pyrrolo[1,2-d][1,2,4]triazin-4-amine formate |
| 23 | 1-[2-(difluoromethyl)-4-methylphenyl]-7-methyl-N-[(3R)-1- |
| | methylpiperidin-3-yl]pyrrolo[1,2-d][1,2,4]triazin-4-amine formate |
| 24 | 1-[4-cyclopropyl-2-(difluoromethoxy)phenyl]-7-methyl-N-[(3R)-1- |
| | methylpiperidin-3-yl]pyrrolo[1,2-d][1,2,4]triazin-4-amine formate |
| 25 | 1-[2-(difluoromethoxy)-4-methylphenyl]-7-methyl-N-[(3R)-1- |
| | methylpiperidin-3-yl]pyrrolo[1,2-d][1,2,4]triazin-4-amine formate |
| 34 | 1-[2-(difluoromethoxy)-4-methylphenyl]-N-[(3R)-1-ethylpiperidin- |
| | 3-yl]pyrrolo[1,2-d][1,2,4]triazin-4-amine formate |
| 35 | 1-[2-(difluoromethoxy)-4-(trifluoromethyl)phenyl]-N-[(3R)- |
| | piperidin-3-yl]pyrrolo[1,2-d][1,2,4]triazin-4-amine hydrochloride |
| 37 | 1-[4-cyclopropyl-2-(trifluoromethoxy)phenyl]-N-[(3R)-1- |
| | methylpiperidin-3-yl]pyrrolo[1,2-d][1,2,4]triazin-4-amine formate |
| 42 | 1-[4-ethyl-2-(trifluoromethoxy)phenyl]-N-[(3R)-1-methylpiperidin- |
| | 3-yl]pyrrolo[1,2-d][1,2,4]triazin-4-amine formate |
| 74 | 1-[4-methoxy-2-(trifluoromethoxy)phenyl]-N-{[(2S)-1- |
| | methylpyrrolidin-2-yl]methyl}pyrrolo[1,2-d][1,2,4]triazin-4-amine |
| | trifluoroacetate |
| 95 | 1-[3-(difluoromethoxy)-4-(4-{[(3R)-1-methylpiperidin-3- |
| | yl]amino}pyrrolo[1,2-d][1,2,4]triazin-1-yl)phenyl]propan-1-one |
| | formate |
| 134 | (3S,4R)-3-({1-[2,4-bis(trifluoromethyl)phenyl]-7-methylpyrrolo[1,2- |
| | d][1,2,4]triazin-4-yl}amino)-1-ethylpiperidin-4-ol formate |
| 145 | 1-[4-methoxy-2-(2,2,2-trifluoroethyl)phenyl]-N-[(3R)-1- |
| | methylpiperidin-3-yl]pyrrolo[1,2-d][1,2,4]triazin-4-amine formate |
| 184 | 2-[(3R)-3-({1-[2-(difluoromethoxy)-4-methylphenyl]imidazo[1,5- |
| | d][1,2,4]triazin-4-yl}amino)piperidin-1-yl]ethan-1-ol formate |
| 185 | 2-[(3R)-3-({1-[4-cyclopropyl-2- |
| | (difluoromethoxy)phenyl]imidazo[1,5-d][1,2,4]triazin-4- |
| | yl}amino)piperidin-1-yl]ethan-1-ol formate |
| 186 | 2-[(3R)-3-({1-[2-(difluoromethoxy)-4- |
| | (trifluoromethyl)phenyl]imidazo[1,5-d][1,2,4]triazin-4- |
| | yl}amino)piperidin-1-yl]ethan-1-ol formate |
| 190 | 1-[4-chloro-2-(difluoromethoxy)phenyl]-N-[(3R)-1-methylpiperidin- |
| | 3-yl]imidazo[1,5-d][1,2,4]triazin-4-amine formate |
| 191 | 1-[4-chloro-2-(difluoromethoxy)phenyl]-N-[(3R)-1-ethylpiperidin-3- |
| | yl]imidazo[1,5-d][1,2,4]triazin-4-amine formate |
| 192 | 2-[(3R)-3-({1-[4-chloro-2-(difluoromethoxy)phenyl]imidazo[1,5- |
| | d][1,2,4]triazin-4-yl}amino)piperidin-1-yl]ethan-1-ol formate |
| 193 | 1-[4-chloro-2-(difluoromethoxy)phenyl]-N-[(3R,5R)-5-fluoro-1- |
| | methylpiperidin-3-yl]imidazo[1,5-d][1,2,4]triazin-4-amine formate |
| 216 | 1-[2-(difluoromethoxy)-4-methoxyphenyl]-N-[(3R)-1- |
| | methylpiperidin-3-yl]imidazo[1,5-d][1,2,4]triazin-4-amine formate |
| 228 | 1-[4-methoxy-2-(trifluoromethyl)phenyl]-N-(1H-pyrazol-3- |
| | yl)imidazo[1,5-d][1,2,4]triazin-4-amine formate |
| 250 | 1-[4-methoxy-2-(2,2,2-trifluoroethyl)phenyl]-N-[(3R)-1- |
| | methylpiperidin-3-yl]imidazo[1,5-d][1,2,4]triazin-4-amine formate |
| 261 | N-[(3R)-1-ethylpiperidin-3-yl]-1-[2-(2,2,2-trifluoroethyl)-4- |
| | (trifluoromethyl)phenyl]imidazo[1,5-d][1,2,4]triazin-4-amine |
| | formate |
| 278 | 2-[(3R)-3-({1-[4-methoxy-2-(2,2,2- |
| | trifluoroethyl)phenyl]imidazo[1,5-d][1,2,4]triazin-4- |
| | yl}amino)piperidin-1-yl]ethan-1-ol formate |
| 279 | 8-methyl-N-[(3R)-1-methylpiperidin-3-yl]-1-[2-(2,2,2- |
| | trifluoroethyl)-4-(trifluoromethyl)phenyl]imidazo[1,5- |
| | d][1,2,4]triazin-4-amine formate |
| 281 | 1-[4-methoxy-2-(pentafluoroethyl)phenyl]-N-[(3R)-1- |
| | methylpiperidin-3-yl]imidazo[1,5-d][1,2,4]triazin-4-amine formate |
| 317 | N-[(3R)-1-methylpiperidin-3-yl]-1-[4-methyl-2-(2,2,2- |
| | trifluoroethyl)phenyl]imidazo[1,5-d][1,2,4]triazin-4-amine formate |
| 357 | 4-[4-bromo-2-(trifluoromethoxy)phenyl]-N-[(3R)-1-ethylpiperidin- |
| | 3-yl]-2-methylpyrazolo[1,5-d][1,2,4]triazin-7-amine formate |
| 358 | 4-[4-chloro-2-(difluoromethoxy)phenyl]-2-methyl-N-[(3R)-1- |
| | methylpiperidin-3-yl]pyrazolo[1,5-d][1,2,4]triazin-7-amine formate |
| 359 | 4-[2-(difluoromethyl)-4-(trifluoromethyl)phenyl]-2-methyl-N-[(3R)- |
| | 1-methylpiperidin-3-yl]pyrazolo[1,5-d][1,2,4]triazin-7-amine |
| | formate |
| 360 | 4-[4-cyclopropyl-2-(difluoromethoxy)phenyl]-2-methyl-N-[(3R)-1- |
| | methylpiperidin-3-yl]pyrazolo[1,5-d][1,2,4]triazin-7-amine formate |
| 361 | 4-[2-(difluoromethoxy)-4-methylphenyl]-2-methyl-N-[(3R)-1- |
| | methylpiperidin-3-yl]pyrazolo[1,5-d][1,2,4]triazin-7-amine formate |
| 363 | 4-[4-cyclopropyl-2-(difluoromethyl)phenyl]-2-methyl-N-[(3R)-1- |
| | methylpiperidin-3-yl]pyrazolo[1,5-d][1,2,4]triazin-7-amine formate |
| 364 | 4-[2-(difluoromethyl)-4-methylphenyl]-2-methyl-N-[(3R)-1- |
| | methylpiperidin-3-yl]pyrazolo[1,5-d][1,2,4]triazin-7-amine formate |
| 368 | N-[(3R)-1-ethylpiperidin-3-yl]-4-[2-(methanesulfonyl)-4- |
| | methylphenyl]-2-methylpyrazolo[1,5-d][1,2,4]triazin-7-amine |
| | trifluoroacetate |
| 369 | N-[(3R)-1-ethylpiperidin-3-yl]-4-[4-(methanesulfonyl)-2- |
| | (trifluoromethoxy)phenyl]-2-methylpyrazolo[1,5-d][1,2,4]triazin-7- |
| | amine trifluoroacetate |
| 372 | 2-(7-{[(3R)-1-ethylpiperidin-3-yl]amino}-2-methylpyrazolo[1,5- |
| | d][1,2,4]triazin-4-yl)-5-(trifluoromethyl)benzamide formate |

wherein a form of the compound is selected from the group consisting of a pharmaceutically acceptable salt, hydrate, solvate, racemate, enantiomer, diastereomer, stereoisomer, tautomer, free-base and isotope enriched form thereof.

4. A pharmaceutical composition comprising a therapeutically effective amount of a compound according to claim 1 or a pharmaceutically acceptable salt thereof and one or more pharmaceutically acceptable carriers.

5. A method for treating or ameliorating a disease modulated by NLRP3 in a subject in need thereof comprising, administering to the subject an effective amount of the compound according to claim 1.

6. A method of treating or ameliorating a disease modulated by NLRP3 according to claim 5 selected from Alzheimer disease, Frontotemporal dementia (FTD), Huntington's disease, Parkinson's disease, Perioperative neurocognitive disorders, Post-cardiac arrest cognitive impairment, Poststroke cognitive impairment, Sepsis, Sepsis associated encephalopathy, Subarachnoid hemorrhage, Macular Degeneration, Retinal neovascularization, Uveitis, Colitis, Endothelial dysfunction, Gout, Pseudogout, Graft-versus-host-disease (GvHD), Systemic lupus erythematosus-lupus nephritis, Cryopyrin-associated periodic syndromes (CAPS), Cystic fibrosis, Sickle-cell disease, VCP-associated disease, Liver fibrosis, Nonalcoholic fatty liver disease (NASH), muscle atrophy, inherited and acquired myopathies, e.g. Duchenne Muscular Dystrophy (DMD), Hyperalgesia, Multiple sclerosis-associated neuropathic pain, Acute Kidney Injury, Chronic crystal nephropathy, Chronic Kidney Disease, asthma and allergic airway inflammation Diabetes-associated atherosclerosis, Diabetic encephalopathy, Diabetic kidney disease, Islet transplantation rejection, Obesity-associated renal disease, Oxalate-induced nephropathy, Renal fibrosis, Renal hypertension, Type I diabetes, Type IL diabetes, Psoriasis, Hidradenitis suppurativa, Atherosclerosis and Cytokine Release Syndrome (CRS).

7. The method of claim 5, wherein the effective amount of the compound is in a range of from about 0.001 mg/kg/day to about 500 mg/kg/day.

8. A compound according to claim 1 or a pharmaceutically acceptable salt thereof, for use in treating or ameliorating a disease modulated by NLRP3 selected from Alzheimer disease, Frontotemporal dementia (FTD), Huntington's disease, Parkinson's disease, Perioperative neurocognitive disorders, Post-cardiac arrest cognitive impairment, Poststroke cognitive impairment, Sepsis, Sepsis associated encephalopathy, Subarachnoid hemorrhage, Macular Degeneration, Retinal neovascularization, Uveitis, Colitis, Endothelial dysfunction, Gout, Pseudogout, Graft-versus-host-disease (GvHD), Systemic lupus erythematosus-lupus nephritis, Cryopyrin-associated periodic syndromes (CAPS), Cystic fibrosis, Sickle-cell disease, VCP-associated disease, Liver fibrosis, Nonalcoholic fatty liver disease (NASH), muscle atrophy, inherited and acquired myopathies, Hyperalgesia, Multiple sclerosis-associated neuropathic pain, Acute Kidney Injury, Chronic crystal nephropathy, Chronic Kidney Disease, asthma and allergic airway inflammation Diabetes-associated atherosclerosis, Diabetic encephalopathy, Diabetic kidney disease, Islet transplantation rejection, Obesity-associated renal disease, Oxalate-induced nephropathy, Renal fibrosis, Renal hypertension, Type I diabetes, Type II diabetes, Psoriasis, Hidradenitis suppurativa, Atherosclerosis and Cytokine Release Syndrome (CRS).

9. Use of a compound according to claim 8, wherein the effective amount of the compound is in a range of from about 0.001 mg/kg/day to about 500 mg/kg/day.

10. Use of a compound according to claim 1 in the preparation of a pharmaceutical composition for treating or ameliorating a disease modulated by NLRP3 in a subject in need thereof comprising, administering to the subject an effective amount of the compound or a form thereof in admixture with one or more of the pharmaceutically acceptable excipients.
